(19) United States
(12) Patent Application Publication
(10) Pub. No.: US 2026/0258064 A1
(43) Pub. Date: Sep. 3, 2026

(54) SARM1 MODULATORS, PREPARATIONS, AND USES THEREOF

(71) Applicant: SIRONAX LTD., Grand Cayman (KY)

(72) Inventors: Zhen SUN, Shanghai (CN); Lianzhu LIU, Shanghai (CN); Yingtao LIU, Shanghai (CN); Baochuan SUN, Beijing (CN); Ming JIANG, Beijing (CN); Jinbao WU, Beijing (CN); Jianguang HAN, Beijing (CN); Zhaolan ZHANG, Beijing (CN); Weidong SUN, Beijing (CN); Bo LI, Beijing (CN); Yanping XU, Beijing (CN); Yimin JIANG, Beijing (CN); Min ZHENG, Shanghai (CN); Xiaofei YANG, Beijing (CN); Bowei YANG, Beijing (CN); Shaoqiang YANG, Beijing (CN); Hanying RUAN, Beijing (CN); Rui YAN, Beijing (CN); Ying LI, Beijing (CN); Zhi HUANG, Beijing (CN); Huihui WU, Beijing (CN); Yong LI, Beijing (CN)

(21) Appl. No.: 19/478,576

(22) PCT Filed: Apr. 26, 2024
(86) PCT No.: PCT/CN2024/089946

§ 371 Date: Oct. 24, 2025

(30) Foreign Application Priority Data
Apr. 27, 2023 (WO) PCT/CN2023/091176

(51) Int. Cl. C07F 9/6558; A61K 31/4184; A61K 31/4192; A61K 31/4196; A61K 31/422; A61K 31/4245; A61K 31/427; A61K 31/433; A61K 31/437; A61K 31/4439; A61K 31/444; A61K 31/496; A61K 31/497; A61K 31/4985; A61K 31/501; A61K 31/506; A61K 31/513; A61K 31/519; A61K 31/53; A61K 31/5377; A61K 31/5386; A61K 31/541; A61K 31/675; C07B 59/00; C07D 401/04; C07D 401/14; C07D 403/04; C07D 403/14; C07D 405/04; C07D 405/14; C07D 409/04; C07D 409/14; C07D 413/04; C07D 413/14; C07D 417/04; C07D 417/14; C07D 471/04; C07D 487/04; C07D 491/107; C07D 495/04; C07D 498/08

(52) U.S. Cl. CPC C07F 9/65583; A61K 31/4184; A61K 31/4192; A61K 31/4196; A61K 31/422; A61K 31/4245; A61K 31/427; A61K 31/433; A61K 31/437; A61K 31/4439; A61K 31/444; A61K 31/496; A61K 31/497; A61K 31/4985; A61K 31/501; A61K 31/506; A61K 31/513; A61K 31/519; A61K 31/53; A61K 31/5377; A61K 31/5386; A61K 31/541; A61K 31/675; C07B 59/002; C07D 401/04; C07D 401/14; C07D 403/04; C07D 403/14; C07D 405/04; C07D 405/14; C07D 409/04; C07D 409/14; C07D 413/04; C07D 413/14; C07D 417/04; C07D 417/14; C07D 471/04; C07D 487/04; C07D 491/107; C07D 495/04; C07D 498/08; C07B 2200/05

(57) ABSTRACT

This disclosure provides compounds of Formula I, compositions comprising the same, and methods of using the same, including uses in modulating SARM1 and treating various diseases and conditions, e.g., those caused by or associated with axonal degeneration.

### RELATED APPLICATION

This application claims priority to International Application No. PCT/CN2023/091176, filed on Apr. 27, 2023, the content of which is incorporated by reference in its entirety.

### FIELD OF THE DISCLOSURE

The present disclosure relates to compounds that modulate SARM1, compositions comprising the compounds, methods of preparing the compounds, and methods of using the compounds to treat various diseases or conditions, e.g., those caused by or associated with axonal degeneration.

### BACKGROUND OF THE DISCLOSURE

Axonal degeneration causes disease progression and accumulation of disability in many degenerative diseases of the peripheral nervous system (PNS) and central nervous systems (CNS), such as multiple sclerosis, Parkinson's disease, and amyotrophic lateral sclerosis (ALS), or acute conditions such as traumatic brain injury. (Hughes 2021 (R. Hughes et al., Small Molecule SARM1 Inhibitors Recapitulate the SARM1−/− Phenotype and Allow Recovery of a Metastable Pool of Axons Fated to Degenerate, Cell Rep. 2021 Jan. 5; 34(1):108588.); Bosanac 2021 (T. Bosanac et al., Pharmacological SARM1 inhibition protects axon structure and function in paclitaxel-induced peripheral neuropathy, Brain, Vol. 144, Issue 10, 2021, pages 3226-3238)). Therefore, axonal protection is an important neuroprotective approach to treatment of chronic and acute CNS and PNS neurodegenerative disorders. (Hughes 2021; Bosanac 2021).

SARM1 (Sterile Alpha and TIR Motif-containing 1) is a unique member of the Myd88 family of adaptor proteins and is considered a major driver of an evolutionarily conserved program of axonal degeneration downstream of chemical, inflammatory, mechanical, or metabolic insults to the axon. (Hughes 2021; Bosanac 2021). SARM1 has been recognized as a central mediator of axonal degeneration in a number of diseases or conditions, including ALS, Parkinson's disease, multiple sclerosis, traumatic brain injury, and diabetic neuropathy, as well as chemotherapy induced peripheral neuropathy (CIPN), which is a major cause of morbidity and the main cause of dose reductions and discontinuations in cancer treatment. (Hughes 2021; Bosanac 2021). SARM1 is a compelling target to treat neurodegeneration characterized by axonopathies of the peripheral and central nervous systems.

SARM1 contains a mitochondrial targeting sequence, an N-terminal domain with armadillo repeats (ARM), two sterile α-motif (SAM) domains, and a Toll/interleukin-1 receptor (TIR) domain (Gerdts 2013 (J. Gerdts et al., Sarm1-mediated axon degeneration requires both SAM and TIR interactions. J Neurosci. 2013 Aug. 14; 33(33):13569-80.)) The SARM1 TIR domain is a NAD⁺ hydrolase (NADase), which converts the NAD⁺ to ADPR or cADPR and NAM (Sporny 2019 (M. Sporny et al., Structural Evidence for an Octameric Ring Arrangement of SARM1. J Mol Biol. 2019 Sep. 6; 431(19):3591-3605.)). This NADase activity is essential for its axonal degenerative function. (Bosanac 2021). The activity of SARM1 also depends on the oligomerization formed through the SAM domains (Sporny 2019) and is autoinhibited by the ARM domain (Chen (2021) (C. Shen et al., Multiple domain interfaces mediate SARM1 autoinhibition. Proc Natl Acad Sci USA. 2021 Jan. 26; 118 (4).)).

Certain SARM1 inhibitors are disclosed by Bosanac 2021, Hughes 2021, Sporny 2020 (M. Sporny et al, Structural basis for SARM1 inhibition and activation under energetic stress. Elife. 2020 Nov. 13; 9:e62021. doi: 10.7554/eLife.62021. PMID: 33185189; PMCID: PMC7688312.), WO 2018/057989 A1, WO 2020/081923 A1, WO 2021/142006 A1, WO2021207302A1, and WO2021207308A1. Certain dipeptidyl peptidase inhibitors (e.g., biphenyl or phenyl benzo imidazole derivatives) are disclosed in US 2005/0272765 A1. Certain benzyl benzoxazol derivatives as Met-kinase inhibitors are disclosed in WO 2008/148449 A1. Certain dihydroisoquinolinone derivatives and combinatorial libraries thereof are described in WO 01/14879. Certain compositions for promoting readthrough of premature termination codons, and methods of using the same are described in WO 2017/049409. Certain nitrogen-containing heterocyclic compounds having nematicidal properties, preparations and uses thereof are described in CN 108276352.

The present disclosure describes SARM1 inhibitors that can be used to prevent axonal degeneration in peripheral and central axonopathies and to provide a transformational disease-modifying treatment for the related diseases or conditions.

### SUMMARY OF THE DISCLOSURE

One aspect of this disclosure provides a compound selected from compounds of Formulae 1, 2, 3-1, 3-2, 3-3, 4, 5, 6-1, 6-2, 7-1, 7-2, 8-1, 8-2, 8-3, 8-4, 8-5, 8-6, 9-1, 9-2, 9-3, 9-4, 10-1, 10-2, 10-3, 10-4, 10-5, 10-6, 11-1, 11-2, 11-3, 11-4, 11-5, 11-6, 11-7, 12-1, 12-2, 12-3, 12-4, 12-5, 12-6, 12-7, and 12-8 (e.g., Compounds 1 to 468) disclosed herein, a tautomer thereof, a solvate or stereoisomer of the compound or the tautomer, or a pharmaceutically acceptable salt of the foregoing, which can be employed in the treatment of various diseases or conditions, such as diseases or conditions caused by or associated with axonal degeneration. For example, disclosed herein is a compound of the following structural Formula 1:

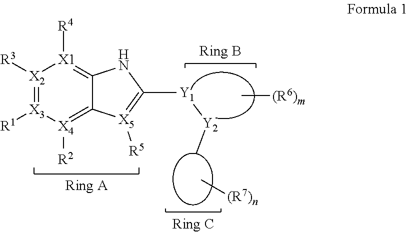

a tautomer thereof, a solvate or stereoisomer of the compound or the tautomer, or a pharmaceutically acceptable salt of the foregoing, wherein:
X₁, X₂, X₃, X₄, and X₅ are each independently C or N;
Y₁ is C or N, Y₂ is C or N, and Y₁ and Y₂ are two adjacent ring atoms on Ring B;
Ring B is phenyl, 5- to 6-membered heteroaryl, 3-6 membered cycloalkyl, or 4- to 6-membered heterocyclyl, wherein the 5- to 6-membered heteroaryl or 4- to 7-membered heterocyclyl of Ring B contains 1 to 4 heteroatoms selected from N, O, and S;
Ring C is phenyl, 3- to 10-membered cycloalkyl, 4- to 10-membered heterocyclyl, 5- to 6-membered heteroaryl, or 9- to 10-membered heteroaryl, wherein the 4- to 10-membered heterocyclyl, 5- to 6-membered heteroaryl, or 9- to 10-membered heteroaryl of Ring C contains 1 to 3 heteroatoms selected from N, S, and O;
R¹ is selected from H, halogen, C₁-C₈ alkyl, C₁-C₈ alkenyl, C₁-C₈ alkynyl, —CN, —OH, —COOH, —C(═O)NH₂, —OR^{m}, —S(═O)ₚ(C₁-C₄ alkyl), —NR^{m}Rⁿ, —C(═O)Rⁿ, —C(═O)OR^{m}, —C(═O)NR^{m}Rⁿ, —P(═O)R^{m}Rⁿ, —SF₅,
   5- to 6-membered heteroaryl containing 1 to 3 heteroatoms independently selected from N, O, and S,
   3- to 10-membered heterocyclyl containing 1 to 2 heteroatoms independently selected from N, O, and S, and
   3- to 10-membered cycloalkyl, wherein:
      the C₁-C₈ alkyl, C₁-C₈ alkenyl, or C₁-C₈ alkynyl of R¹ is optionally substituted with 1-3 groups selected from halogen, —OH, —OR^{m}, —CN, —NH₂, —NR^{m}Rⁿ, —C(═O)OCH₃, —O(C₁-C₆ alkyl), —COOH, —C(═O)NH₂, phenyl, 5 to 6-membered heteroaryl, 3 to 6-heterocyclyl, and 3- to 6-membered cycloalkyl (optionally substituted with 1-3 groups selected from OH and halogen),
      the 5- to 6-membered heteroaryl of R¹ is optionally substituted by 1 to 3 groups selected from D, halogen, —OH, —CN, —COOH, —(C₁-C₆ alkyl)OH, —C(═O)O(C₁-C₆ alkyl), ═O, —NH₂, —C(═O)NR^{m}Rⁿ, 5- to 6-membered heteroaryl, —OR^{m}, R^{m}, C₁-C₆ alkyl (optionally substituted with 1-3 groups selected from halogen, —C(═O)NH₂, R^{m}, and OR^{m}),
      the 3- to 10-membered heterocyclyl of R¹ is optionally substituted by 1 to 3 groups selected from D, halogen, —OH, —CN, —COOH, —(C₁-C₆ alkyl)OH, —C(═O)O(C₁-C₆ alkyl), ═O, —NH₂, —C(═O)NR^{m}Rⁿ, 5- to 6-membered heteroaryl, —OR^{m}, R^{m}, C₁-C₆ alkyl (optionally substituted with 1-3 groups selected from halogen, —C(═O)NH₂, R^{m}, and OR^{m}), and
      the 3- to 10-membered cycloalkyl of R¹ is optionally substituted by 1 to 3 groups selected from D, halogen, —OH, —CN, —COOH, —(C₁-C₆ alkyl)OH, —C(═O)O(C₁-C₆alkyl), ═O, —NH₂, —C(═O)NR^{m}Rⁿ, 5- to 6-membered heteroaryl, —OR^{m}, R^{m}, C₁-C₆ alkyl (optionally substituted with 1-3 groups selected from halogen, —C(═O)NH₂, R^{m}, and OR^{m}), and
   wherein R^{m} and Rⁿ, for each occurrence, are each independently selected from H, C₁-C₆ alkyl, —S(═O)ₚ(C₁-C₄ alkyl), phenyl, 3- to 8-membered cycloalkyl, 4- to 6-membered heterocyclyl, and 5- to 6-membered heteroaryl, wherein the C₁-C₆ alkyl of R^{m} is optionally substituted with 1-3 groups selected from D, —C(═O)NH₂, —OH, —OMe, —S(═O)₂CH₃, and halogen;
R² is selected from H, halogen, C₁-C₆ alkyl, C₁-C₆ alkenyl, —OH, —O(C₁-C₆ alkyl), —O(C₁-C₆ alkyl)O(C₁-C₆ alkyl), —C(═O)NH₂, —S(═O)ₚ(C₁-C₄ alkyl), —CN, 3- to 6-membered cycloalkyl, phenyl, 5- to 6-membered heteroaryl, and 4- to 10-membered heterocyclyl (containing 1 to 3 heteroatoms independently selected from S, O, and N), wherein:
   the C₁-C₆ alkyl or C₁-C₆ alkenyl of R² is optionally substituted with 1-3 groups selected from halogen, CN, and —C(═O)O(C₁-C₆ alkyl),
   the 3- to 5-membered cycloalkyl of R² is optionally substituted with 1-3 groups selected from OH, CN, and halogen,
   the C₁-C₆ alkyl of the —O(C₁-C₆ alkyl) of R² is optionally substituted with 1-3
   groups selected from halogen and CN, and the 3- to 10-membered heterocyclyl of R² is optionally substituted with 1-3 groups selected from OH, CN, and halogen; or
R¹ and R² join to form

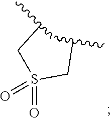

R³ and R⁴ are each independently selected from H, halogen, C₁-C₆ alkyl (optionally substituted with 1-3 groups selected from OH and halogen), and —O(C₁-C₆ alkyl);
R⁵ is selected from absent, H, —CN, halogen, —C(═O)NH₂, —S(═O)ₚ(C₁-C₄ alkyl), —OR^{p}, phenyl, 5- to 6-membered heteroaryl, 4- to 6-membered heterocyclyl, 3- to 8-membered cycloalkyl, and C₁-C₆ alkyl, wherein:
   the C₁-C₆ alkyl of R¹ is optionally substituted with 1 to 3 groups selected from OH, —NHR^{p}, —OR^{p}, and —S(═O)ₚ(C₁-C₄ alkyl),
   the 4- to 6-membered heterocyclyl of R⁵ is optionally substituted with 1 to 3 groups selected from C₁-C₃ alkyl, CN, halogen, and ═O,
   the 3- to 8-membered cycloalkyl of R⁵ is optionally substituted with 1 to 3 groups selected from C₁-C₃ alkyl, CN, and halogen, wherein:
   R^{p} is selected from C₁-C₆ alkyl, 3- to 6-membered cycloalkyl, and 5- to 6-membered heteroaryl, wherein the C₁-C₆ alkyl, 3- to 6-membered cycloalkyl, or 5- to 6-membered heteroaryl of R^{p} is optionally substituted with 1 to 3 groups selected from CN, OH, and halogen;
R⁶, for each occurrence, is independently selected from D, halogen, —CN, ═O, —OR^{s}, —SH, —S(C₁-C₄ alkyl), —S(═O)ₚR^{t}, —C(═O)NR^{t}R^{o}, —NR^{t}R^{o}, 4- to 6-membered heterocyclyl, and C₁-C₆ alkyl, wherein:
   the C₁-C₆ alkyl of R⁶ is optionally substituted with 1 to 3 groups selected from halogen, —OR^{s}, ═O, —S(═O)ₚR^{t}, —NHS(═O)ₚR^{t}, —S(═O)(═NH)R^{t},

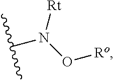

—NHS(═O)ₚ(C₁-C₄ alkyl), —CN, —C(═O)NR^{t}R^{o}, —NR^{t}R^{o}, halogen, 5- to 6-membered heteroaryl, 3- to 6-membered cycloalkyl (optionally substituted with 1 to 3 groups selected from halogen, OH, and R), and 4- to 10-membered heterocyclyl optionally substituted with 1 to 3 groups selected from halogen, OH, and R^{t}, wherein:
the 4- to 8-membered heterocyclyl of the C₁-C₆ alkyl of R⁶ is optionally substituted with 1 to 3 groups selected from halogen, OH, C₁-C₃ alkyl, and ═O;
R^{s} is selected from H, C₁-C₆ alkyl, 4- to 6-membered heterocyclyl, and 3- to 6-membered cycloalkyl, wherein:
   the C₁-C₆ alkyl of R^{s} is optionally substituted with 1-3 groups selected from —OH, —OMe, and halogen and
   the 3- to 6-membered cycloalkyl of R^{s} is optionally substituted with —OH or —OMe;
R^{t} and R^{o}, for each occurrence, are each independently selected from H, C₁-C₆ alkyl, 5- to 6-membered heteroaryl, 4- to 6-membered heterocyclyl, and 3- to 5-membered cycloalkyl, wherein the C₁-C₆ alkyl of R^{t} and R^{o} is optionally substituted with 1-3 groups selected from D, halogen, —OH, CN, C(═O)NH₂, —O(C₁-C₃ alkyl), and —S(═O)₂CH₃;
R⁷, for each occurrence, is independently selected from D, halogen, —OR^{a}, —CN, —CONH₂, —C(═O)NR^{b}R^{c}, NR^{b}R^{c}, —C(═O)OR^{b}, ═O, ═S, —P(═O)₂R^{b}R^{c}, —S(═O)ₚ(C₁-C₄ alkyl), —O(C₁-C₆ alkyl), C₁-C₆ alkyl, C₁-C₆ alkenyl, C₁-C₆ alkynyl, 3- to 6-membered cycloalkyl, 4- to 6-membered heterocyclyl, and 5- to 6-membered heteroaryl, wherein:
   the C₁-C₆ alkyl, C₁-C₆ alkenyl, or C₁-C₆ alkynyl of R⁷ is optionally substituted with 1 to 3 groups selected from halogen, —OH, CN, —S(═O)ₚ(C₁-C₄ alkyl), —C(═O)₂NH₂, and 3- to 6-membered heterocyclyl,
   the 4- to 6-membered heterocyclyl of R⁷ is optionally substituted with 1 to 3 groups selected from ═O, halogen, and R^{b},
   R^{a} is selected from H, C₁-C₈ alkyl, 3- to 6-membered cycloalkyl, 4- to 6-membered heterocyclyl, phenyl, and 5- to 6-membered heteroaryl, wherein the C₁-C₈ alkyl of R^{a} is optionally substituted with 1 to 4 groups selected from D, halogen, OH, CN, —S(═O)ₚ(C₁-C₄ alkyl), —C(═O)NH₂, 3- to 6-membered cycloalkyl, 4- to 6-membered heterocyclyl, and 5- to 6-membered heteroaryl,
   R^{b} and R^{c}, for each occurrence, are each independently selected from H, C₁-C₈ alkyl, 4- to 6-membered heterocyclyl, phenyl, 5- to 6-membered heteroaryl, and 3- to 6-membered cycloalkyl, wherein the C₁-C₈ alkyl of R^{b} and R^{c} is optionally substituted with 1 to 3 groups selected from D, halogen, OH, —C(═O)NH₂, CN, —OCH₃, and —S(═O)₂CH₃;
m is an integer selected from 0, 1, and 2;
n is an integer selected from 0, 1, 2, 3, and 4; and
p is an integer selected from 0, 1, and 2.

In one aspect of the disclosure, the compounds of the Formulae disclosed herein are selected from Compounds 1 to 468 shown in Table 1, a tautomer thereof, a solvate or stereoisomer of the compound or the tautomer, or a pharmaceutically acceptable salt of the foregoing.

In some embodiments, the disclosure provides pharmaceutical compositions comprising a compound of Formula 1, 2, 3-1, 3-2, 3-3, 4, 5, 6-1, 6-2, 7-1, 7-2, 8-1, 8-2, 8-3, 8-4, 8-5, 8-6, 9-1, 9-2, 9-3, 9-4, 10-1, 10-2, 10-3, 10-4, 10-5, 10-6, 11-1, 11-2, 11-3, 11-4, 11-5, 11-6, 11-7, 12-1, 12-2, 12-3, 12-4, 12-5, 12-6, 12-7, or 12-8 (e.g., Compounds 1 to 468) disclosed herein, a tautomer thereof, a solvate or stereoisomer of the compound or the tautomer, or a pharmaceutically acceptable salt of the foregoing, and a pharmaceutically acceptable carrier. In some embodiments, the pharmaceutical compositions may comprise a compound selected from Compounds 1 to 468 shown below, a tautomer thereof, a solvate or stereoisomer of the compound or the tautomer, or a pharmaceutically acceptable salt of the foregoing, and a pharmaceutically acceptable carrier. These compositions may further comprise an additional active pharmaceutical agent.

Another aspect of the disclosure provides methods of treating a disease or condition, comprising administering to a subject in need thereof, a therapeutically effective amount of a compound of Formula 1, 2, 3-1, 3-2, 3-3, 4, 5, 6-1, 6-2, 7-1, 7-2, 8-1, 8-2, 8-3, 8-4, 8-5, 8-6, 9-1, 9-2, 9-3, 9-4, 10-1, 10-2, 10-3, 10-4, 10-5, 10-6, 11-1, 11-2, 11-3, 11-4, 11-5, 11-6, 11-7, 12-1, 12-2, 12-3, 12-4, 12-5, 12-6, 12-7, or 12-8 (e.g., Compounds 1 to 468) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt, wherein the disease or condition is selected amyotrophic lateral sclerosis (ALS), Parkinson's disease, Parkinsonian syndromes, ischemia, stroke, herpes infection, a demyelinating disease such as multiple sclerosis, traumatic brain injury, sepsis, a chronic disease of PNS including inherited neuropathies, such as, but is not limited to Charcot-Marie-Tooth disease and chronic inflammatory demyelinating polyneuropathy (CIDP), an optic nerve disorder such as glaucoma, and retinal ganglion degeneration, colitis a metabolic disease or disorder such as diabetic neuropathy, nonalcoholic fatty liver disease (NAFLD) and nonalcoholic steatohepatitis (NASH) and a peripheral neuropathy like CIPN induced by various drugs.

A further aspect of the disclosure provides methods of treating a disease or condition caused by or associated with axonal degeneration, or neuronal damage mediated by SARM1 comprising administering to a subject in need thereof, a therapeutically effective amount of a compound of Formula 1, 2, 3-1, 3-2, 3-3, 4, 5, 6-1, 6-2, 7-1, 7-2, 8-1, 8-2, 8-3, 8-4, 8-5, 8-6, 9-1, 9-2, 9-3, 9-4, 10-1, 10-2, 10-3, 10-4, 10-5, 10-6, 11-1, 11-2, 11-3, 11-4, 11-5, 11-6, 11-7, 12-1, 12-2, 12-3, 12-4, 12-5, 12-6, 12-7, or 12-8 (e.g., Compounds 1 to 468) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt.

In some embodiments, the methods of treatment comprise administering to a subject in need thereof, a compound selected from Compounds 1 to 468 shown below, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt.

In some embodiments, the methods of treatment comprise administration of an additional active pharmaceutical agent to the subject in need thereof, either in the same pharmaceutical composition as a compound of Formula 1, 2, 3-1, 3-2, 3-3, 4, 5, 6-1, 6-2, 7-1, 7-2, 8-1, 8-2, 8-3, 8-4, 8-5, 8-6, 9-1, 9-2, 9-3, 9-4, 10-1, 10-2, 10-3, 10-4, 10-5, 10-6, 11-1, 11-2, 11-3, 11-4, 11-5, 11-6, 11-7, 12-1, 12-2, 12-3, 12-4, 12-5, 12-6, 12-7, or 12-8 (e.g., Compounds 1 to 468) disclosed herein, a tautomer thereof, a solvate or stereoisomer of the compound or the tautomer, or a pharmaceutically acceptable salt of the foregoing, or in a separate composition. In some embodiments, the methods of treatment comprise administering a compound selected from Compounds 1 to 468 shown below, a tautomer thereof, a solvate or stereoisomer of the compound or the tautomer, or a pharmaceutically acceptable salt of the foregoing with an additional active pharmaceutical agent either in the same pharmaceutical composition or in a separate composition. When administered as a separate composition, the additional therapeutic agent may be administered prior to, at the same time as, or following administration of the compound, tautomer, solvate, stereoisomer, or a pharmaceutically acceptable salt disclosed herein.

Also disclosed herein are methods of modulating, e.g., inhibiting, SARM1 in a subject in need thereof, comprising contacting the subject with an effective amount of a compound of Formula 1, 2, 3-1, 3-2, 3-3, 4, 5, 6-1, 6-2, 7-1, 7-2, 8-1, 8-2, 8-3, 8-4, 8-5, 8-6, 9-1, 9-2, 9-3, 9-4, 10-1, 10-2, 10-3, 10-4, 10-5, 10-6, 11-1, 11-2, 11-3, 11-4, 11-5, 11-6, 11-7, 12-1, 12-2, 12-3, 12-4, 12-5, 12-6, 12-7, or 12-8 (e.g., Compounds 1 to 468) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt. In some embodiments, the methods of modulating, e.g., inhibiting, SARM1 in a subject in need thereof comprise contacting the subject with an effective amount of a compound selected from Compounds 1 to 468 shown below, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt.

Also disclosed herein are methods of inhibiting or preventing axonal degeneration in a subject in need thereof, comprising contacting the subject with an effective amount of a compound of Formula 1, 2, 3-1, 3-2, 3-3, 4, 5, 6-1, 6-2, 7-1, 7-2, 8-1, 8-2, 8-3, 8-4, 8-5, 8-6, 9-1, 9-2, 9-3, 9-4, 10-1, 10-2, 10-3, 10-4, 10-5, 10-6, 11-1, 11-2, 11-3, 11-4, 11-5, 11-6, 11-7, 12-1, 12-2, 12-3, 12-4, 12-5, 12-6, 12-7, or 12-8 (e.g., Compounds 1 to 468) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt. In some embodiments, the methods of inhibiting or preventing axonal degeneration or neuronal damage mediated by SARM1 in a subject in need thereof comprise contacting the subject with an effective amount of a compound selected from Compounds 1 to 468 shown below, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt.

### DETAILED DESCRIPTION OF THE DISCLOSURE

### I. Definitions

The term “a” or “an” when referring to a noun as used herein encompasses the expression “at least one” and therefore encompasses both singular and plural units of the noun. For example, “an additional pharmaceutical agent” means a single or two or more additional pharmaceutical agents.

The term “alkyl” refers to a hydrocarbon group selected from linear and branched saturated hydrocarbon groups, containing 1-20, e.g., 1-18, 1-12, 1-10, 1-8, 1-6, 1-4, or 1-3, carbon atoms. Examples of the alkyl group include methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), and 1,1-dimethylethyl or t-butyl (“t-Bu”). Other examples of an alkyl group include 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, and 3,3-dimethyl-2-butyl groups. Lower alkyl contains 1-8, preferably 1-6, more preferably 1-4 carbon atoms, and more preferably 1-3 carbon atoms.

The term “alkenyl” refers to a hydrocarbon group selected from linear and branched hydrocarbon groups, comprising at least one C═C double bond and 2-20, e.g., 2-18, 2-12, 2-10, 2-8, 2-6, or 2-4, carbon atoms. Examples of the alkenyl group include ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl, and hexa-1,3-dienyl groups. Lower alkenyl contains 2-8, preferably 2-6, and more preferably 2-4 carbon atoms.

The term “alkynyl” refers to a hydrocarbon group selected from linear and branched hydrocarbon groups, comprising at least one C≡C triple bond and 2-20, e.g., 2-18, 2-12, 2-10, 2-8, 2-6, or 2-4, carbon atoms. Examples of the alkynyl group include ethynyl, 1-propynyl, 2-propynyl (propargyl), 1-butynyl, 2-butynyl, and 3-butynyl groups. Lower alkynyl contains 2-8, preferably 2-6, and more preferably 2-4 carbon atoms.

The term “heteroalkyl” refers to an alkyl group, as defined herein, in which one or more of the constituent carbon atoms have been replaced by a heteroatom, e.g., nitrogen, oxygen, or sulfur, e.g., CH₃CH₂OH, CH₃CH₂OC₂H₅, CH₃CH₂SH, CH₃CH₂SC₂H₅, CH₃CH₂NH₂, CH₃CH₂NHC₂H₅, etc. In some embodiments, in addition to the replacement of one or more of the constituent carbon atoms by nitrogen, oxygen, or sulfur, a heteroalkyl group is further optionally substituted as defined herein.

The term “cycloalkyl” refers to a hydrocarbon group selected from saturated and partially unsaturated cyclic hydrocarbon groups, e.g., monocyclic and polycyclic (e.g., bicyclic and tricyclic) groups. For example, the cycloalkyl group may be of 3-12, 3-10, 3-8, 3-6, 3-4, or 5-6 carbon atoms. Even further for example, the cycloalkyl group may be a monocyclic group of 3-12, 3-8, 3-6, 3-4, or 5-6 carbon atoms. Examples of the monocyclic cycloalkyl group include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, and cyclododecyl groups. Examples of the bicyclic cycloalkyl groups include those having 7-12 ring atoms arranged as a bicycle ring selected from [4,4], [4,5], [5,5], [5,6], and [6,6] ring systems, or as a bridged bicyclic ring selected from bicyclo[2.2.1]heptane, bicyclo[2.2.2]octane, and bicyclo[3.2.2]nonane. The ring may be saturated or have at least one double bond (i.e., partially unsaturated), but is not fully conjugated, and is not an aromatic ring, as “aromatic ring” is defined herein.

The term “heterocyclic” or “heterocycle” or “heterocyclyl” refers to a ring selected from 3- to 12-membered, e.g., 3- to 6-membered, 3- to 5-membered, 4- to 5-membered, or 5- to 6-membered, monocyclic, bicyclic, and tricyclic, saturated and partially unsaturated rings comprising at least one carbon atom in addition to 1, 2, 3, or 4 heteroatoms, selected from, e.g., oxygen, sulfur, nitrogen, and silicon. “Heterocycle” also refers to a 5- to 7-membered heterocyclic ring comprising at least one heteroatom selected from N, O, and S fused with 5-, 6-, and/or 7-membered cycloalkyl, carbocyclic aromatic, or heteroaromatic ring, provided that the point of attachment is at the heterocyclic ring when the heterocyclic ring is fused with a carbocyclic aromatic or a heteroaromatic ring, and that the point of attachment can be at the cycloalkyl or heterocyclic ring when the heterocyclic ring is fused with cycloalkyl.

“Heterocycle” also refers to an aliphatic spirocyclic ring comprising at least one heteroatom selected from N, O, and S, provided that the point of attachment is at the heterocyclic ring. The rings may be saturated or have at least one double bond (i.e., partially unsaturated). A heterocycle may be substituted with oxo. The point of the attachment may be carbon or heteroatom in the heterocyclic ring. A heterocycle is not a heteroaryl as defined herein.

Examples of heterocycles include, but are not limited to, (as numbered from the linkage position assigned priority 1) 1-pyrrolidinyl, 2-pyrrolidinyl, 2,4-imidazolidinyl, 2,3-pyrazolidinyl, 1-piperidinyl, 2-piperidinyl, 3-piperidinyl, 4-piperidinyl, 2,5-piperazinyl, pyranyl, 2-morpholinyl, 3-morpholinyl, oxiranyl, aziridinyl, thiiranyl, azetidinyl, oxetanyl, thietanyl, 1,2-dithietanyl, 1,3-dithietanyl, dihydropyridinyl, tetrahydropyridinyl, thiomorpholinyl, thioxanyl, piperazinyl, homopiperazinyl, homopiperidinyl, azepanyl, oxepanyl, thiepanyl, 1,4-oxathianyl, 1,4-dioxepanyl, 1,4-oxathiepanyl, 1,4-oxaazepanyl, 1,4-dithiepanyl, 1,4-thiazepanyl, 1,4-diazepanyl, 1,4-dithianyl, 1,4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, 1,4-dioxanyl, 1,3-dioxolanyl, pyrazolinyl, pyrazolidinyl, dithianyl, dithiolanyl, pyrazolidinylimidazolinyl, pyrimidinonyl, 1,1-dioxo-thiomorpholinyl, 3-azabicyco[3.1.0]hexanyl, 3-azabicyclo[4.1.0]heptanyl and azabicyclo[2.2.2]hexanyl. Substituted heterocycle also includes ring systems substituted with one or more oxo moieties, such as piperidinyl N-oxide, morpholinyl-N-oxide, 1-oxo-1-thiomorpholinyl, and 1, 1-dioxo-1-thiomorpholinyl.

The term “fused ring” herein refers to a polycyclic ring system, e.g., a bicyclic or tricyclic ring system, in which two rings share only two ring atoms and one bond in common. Examples of fused rings may comprise a fused bicyclic cycloalkyl ring such as those having from 7 to 12 ring atoms arranged as a bicyclic ring selected from [4,4], [4,5], [5,5], [5,6], and [6,6] ring systems as mentioned above; a fused bicyclic aryl ring such as 7- to 12-membered bicyclic aryl ring systems as mentioned above, a fused tricyclic aryl ring such as 10- to 15-membered tricyclic aryl ring systems mentioned above; a fused bicyclic heteroaryl ring such as 8- to 12-membered bicyclic heteroaryl rings as mentioned above, a fused tricyclic heteroaryl ring such as 11- to 14-membered tricyclic heteroaryl rings as mentioned above; and a fused bicyclic or tricyclic heterocyclyl ring as mentioned above.

The term “heteroatom” means one or more of oxygen, sulfur, nitrogen, phosphorus, and silicon, including, any oxidized form of nitrogen or sulfur; the quaternized form of any basic nitrogen or a substitutable nitrogen of a heterocyclic ring, for example N (as in 3,4-dihydro-2H-pyrrolyl), NH (as in pyrrolidinyl) or NR⁺ (wherein R is, e.g., an optionally substituted alkyl group) (as in N-substituted pyrrolidinyl).

The term “unsaturated”, as used herein, means that a moiety has one or more units or degrees of unsaturation. Unsaturation is the state in which not all of the available valence bonds in a compound are satisfied by substituents and thus the compound contains one or more double or triple bonds. A double bond may be depicted as [symbol] (two solid lines). The depiction of [symbol] (a solid line and a dashed line), as used herein, denotes a bond that may be a double bond or a single bond.

The term “alkoxy” as used herein, refers to an alkyl group, as defined above, wherein one carbon of the alkyl group is replaced by an oxygen atom, provided that the oxygen atom is linked between two carbon atoms.

The term “halogen” includes F, Cl, Br, and I, i.e., fluoro, chloro, bromo, and iodo, respectively.

As used herein, a “CN,” “cyano” or “nitrile” group refers to —C≡N.

As used herein, an “aromatic ring” refers to a carbocyclic or heterocyclic ring that contains conjugated, planar ring systems with delocalized pi electron orbitals comprised of [4n+2]p orbital electrons, wherein n is an integer of 0 to 6. A “non-aromatic” ring refers to a carbocyclic or heterocyclic that does not meet the requirements set forth above for an aromatic ring, and can be either completely or partially saturated. Non-limiting examples of aromatic rings include aryl and heteroaryl rings that are further defined as follows. An “aromatic ring” may be depicted as a cycle with conjugated double bonds, such as

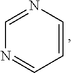

or as a cycle with an inside circle, such as

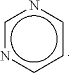

The term “aryl” herein refers to a group selected from: monocyclic carbocyclic aromatic rings, for example, phenyl; bicyclic ring systems such as 7-12 membered, e.g., 9-10 membered, bicyclic ring systems wherein at least one ring is carbocyclic and aromatic, selected, for example, from naphthalene, indane, and 1,2,3,4-tetrahydroquinoline; and tricyclic ring systems such as 10-15 membered tricyclic ring systems wherein at least one ring is carbocyclic and aromatic, for example, fluorene.

For example, the aryl group may be a 6-membered carbocyclic aromatic ring fused to a 5- to 7-membered cycloalkyl or heterocyclic ring optionally comprising at least one heteroatom selected from N, O, and S, provided that the point of attachment is at the carbocyclic aromatic ring when the carbocyclic aromatic ring is fused with a heterocyclic ring, and the point of attachment can be at the carbocyclic aromatic ring or at the cycloalkyl group when the carbocyclic aromatic ring is fused with a cycloalkyl group. Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene.

The term “heteroaryl” refers to a group selected from: 5- to 7-membered, e.g., 5- to 6-membered, aromatic, monocyclic rings comprising 1, 2, 3, or 4 heteroatoms selected from N, O, and S, with the remaining ring atoms being carbon; 8- to 12-membered bicyclic rings comprising 1, 2, 3, or 4 heteroatoms, selected from N, O, and S, with the remaining ring atoms being carbon and wherein at least one ring is aromatic and at least one heteroatom is present in the aromatic ring; and 11- to 14-membered tricyclic rings comprising 1, 2, 3, or 4 heteroatoms, selected from N, O, and S, with the remaining ring atoms being carbon and wherein at least one ring is aromatic and at least one heteroatom is present in an aromatic ring.

For example, the heteroaryl group may be a 5- to 7-membered heterocyclic aromatic ring fused to a 5- to 7-membered cycloalkyl ring. For such fused, bicyclic heteroaryl ring systems wherein only one of the rings comprises at least one heteroatom, the point of attachment may be at the heteroaromatic ring or at the cycloalkyl ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1.

Examples of the heteroaryl group include, but are not limited to, (as numbered from the linkage position assigned priority 1) pyridyl (such as 2-pyridyl, 3-pyridyl, or 4-pyridyl), cinnolinyl, pyrazinyl, 2,4-pyrimidinyl, 3,5-pyrimidinyl, 2,4-imidazolyl, imidazopyridinyl, isoxazolyl, oxazolyl, thiazolyl, isothiazolyl, thiadiazolyl, tetrazolyl, thienyl, triazinyl, benzothienyl, furyl, benzofuryl, benzoimidazolyl, indolyl, isoindolyl, indolinyl, phthalazinyl, pyrazinyl, pyridazinyl, pyrrolyl, triazolyl, quinolinyl, isoquinolinyl, pyrazolyl, pyrrolopyridinyl (such as 1H-pyrrolo[2,3-b]pyridin-5-yl), pyrazolopyridinyl (such as 1H-pyrazolo[3,4-b]pyridin-5-yl), benzoxazolyl (such as benzo[d]oxazol-6-yl), pteridinyl, purinyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, furazanyl, benzofurazanyl, benzothiophenyl, benzothiazolyl, benzoxazolyl, quinazolinyl, quinoxalinyl, naphthyridinyl, furopyridinyl, benzothiazolyl (such as benzo[d]thiazol-6-yl), indazolyl (such as 1H-indazol-5-yl) and 5,6,7,8-tetrahydroisoquinolinyl.

The term “acyl” refers to a substituent group where a point of attachment in the substituent group is a carbonyl. Exemplary acyl groups include, but are not limited to, —C(═O)R′, —C(═O)NR′R″, or —C(═O)OR′, wherein R′ and R″ are independently selected from hydrogen, alkyl, alkenyl, alkynyl, heteroalkyl, heteroalkenyl, heteroalkynyl, cycloalkyl, aryl, heterocyclyl, or heteroaryl, any of which may be further substituted by one or more substituents.

Some of the compounds may exist with different points of attachment of hydrogen, referred to as “tautomers.” For example, compounds including carbonyl —CH₂C(O)— groups (keto forms) may undergo tautomerism to form hydroxyl —CH═C(OH)— groups (enol forms). Both keto and enol forms, individually as well as mixtures thereof, are also intended to be included where applicable. For example,

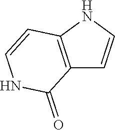

is considered a tautomeric form of

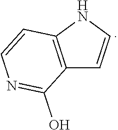

The compounds, tautomers, solvates, or pharmaceutically acceptable salts of the disclosure may contain an asymmetric center and may thus exist as enantiomers. For example, where the compounds possess two or more asymmetric centers, they may additionally exist as diastereoisomers. Enantiomers and diastereoisomers fall within the broader class of stereoisomers. All such possible stereoisomers as substantially pure resolved enantiomers, racemic mixtures thereof, as well as mixtures of diastereoisomers are intended to be included in this disclosure. All stereoisomers of the compounds, tautomers, solvates, and pharmaceutically acceptable salts thereof are intended to be included. Unless specifically mentioned otherwise, reference to one isomer applies to any of the possible isomers. Whenever the isomeric composition is unspecified, all possible isomers are included.

Diastereomeric mixtures can be separated into their individual diastereoisomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereoisomers using optically active resolving agents. Racemic mixtures of chiral compounds of the disclosure can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereoisomers, and conversion to the pure stereoisomers, and (3) separation of the substantially pure or enriched stereoisomers directly under chiral conditions.

In the present disclosure, certain single stereoisomers, e.g., substantially pure enantiomers, are separated from one another, e.g., by a chiral separation. However, the absolute configuration of certain separated single stereoisomers are not presently known. For instance, The compounds in Examples 186 and 187 were synthesized and separated by chiral separation and the compounds are indicated as “single unknown stereoisomer.” As a further example, the compounds in Examples 433 and 434 were synthesized and separated by chiral separation and the compounds are indicated as “single unknown enantiomer” and the stereocenters are noted as “or 1.”

The term “substantially pure” in the context of stereoisomers means that the target stereoisomer contains no more than 35%, such as no more than 30%, further such as no more than 25%, even further such as no more than 20%, by weight of any other stereoisomer(s). In some embodiments, the term “substantially pure” means that the target stereoisomer contains no more than 10%, for example, no more than 5%, such as no more than 1%, by weight of any other stereoisomer(s).

Unless otherwise indicated, structures depicted herein are meant to include all isomeric forms of the structure, e.g., racemic mixtures, cis/trans isomers, geometric (or conformational) isomers, such as (Z) and (E) double bond isomers, and (Z) and (E) conformational isomers. Therefore, geometric and conformational mixtures of the compounds disclosed herein are within the scope of the disclosure. Unless otherwise stated, all tautomeric forms of the compounds of the disclosure are within the scope of the disclosure.

The disclosure provides pharmaceutically acceptable salts of the disclosed compounds, tautomers, solvates, and stereoisomers. A salt of a compound is formed between an acid and a basic group of the compound, such as an amino functional group, or a base and an acidic group of the compound, such as a carboxyl functional group.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this disclosure.

“Pharmaceutically acceptable salts” include, but are not limited to salts with inorganic acids, selected, for example, from hydrochlorates, phosphates, diphosphates, hydrobromates, sulfates, sulfinates, and nitrates; as well as salts with organic acids, selected, for example, from malates, maleates, fumarates, tartrates, succinates, citrates, lactates, methanesulfonates, p-toluenesulfonates, 2-hydroxyethylsulfonates, benzoates, salicylates, stearates, alkanoates such as acetate, and salts with HOOC—(CH₂)n-COOH, wherein n is selected from 0 to 4. Similarly, examples of pharmaceutically acceptable cations include, but are not limited to, sodium, potassium, calcium, magnesium, aluminum, lithium, and ammonium. Suitable pharmaceutically acceptable salts are, for example, those disclosed in S. M. Berge, et al. J. Pharmaceutical Sciences, 1977, 66, pp. 1 to 19.

Acids commonly employed to form pharmaceutically acceptable salts include inorganic acids such as hydrogen bisulfide, hydrochloric acid, hydrobromic acid, hydroiodic acid, sulfuric acid, and phosphoric acid, as well as organic acids such as para-toluenesulfonic acid, salicylic acid, tartaric acid, bitartaric acid, ascorbic acid, maleic acid, benzenesulfonic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid, and acetic acid. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate (i.e., caprate), caprylate, acrylate, formate, isobutyrate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, tartrate, methanesulfonate, propanesulfonate, naphthalene-1-sulfonate, naphthalene-2-sulfonate, mandelate, and other salts. In some embodiments, pharmaceutically acceptable acid addition salts include those formed with mineral acids such as hydrochloric acid and hydrobromic acid, and those formed with organic acids such as maleic acid.

Pharmaceutically acceptable salts derived from appropriate bases include alkali metal, alkaline earth metal, ammonium, and N⁺(C₁₋₄ alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium salts. Further non-limiting examples of pharmaceutically acceptable salts include salts of ammonium, quaternary ammonium, and amine cations formed using counterions such as halide, hydroxide, carboxylate, sulfate, phosphate, nitrate, lower alkyl sulfonate, and aryl sulfonate. Other suitable, non-limiting examples of pharmaceutically acceptable salts include besylate and glucosamine salts.

If a compound is obtained as an acid addition salt, the free base can be obtained by basifying a solution of the acid addition salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

The compounds, tautomers, solvates, stereoisomers, and pharmaceutically acceptable salts of the disclosure may also contain unnatural proportions of atomic isotopes at one or more of the atoms that constitute such compounds. For example, —CD₃, —CD₂H or —CDH₂ contains one or more deuteriums in place of hydrogen. For example, the compounds may be radiolabeled with radioactive isotopes, such as for example tritium (³H), iodine-125 (¹²⁵I), or carbon-14 (¹⁴C). All isotopic variations of the compounds of the disclosure, whether radioactive or not, are intended to be encompassed within the scope of the disclosure.

As used herein, “optionally substituted” is interchangeable with the phrase “substituted or unsubstituted.” In general, the term “substituted,” refers to the replacement of a hydrogen radical in a given structure with the radical of a specified substituent. Unless otherwise indicated, an “optionally substituted” group may have a substituent at each substitutable position of the group, and when more than one position in any given structure may be substituted with more than one substituent chosen from a specified group, the substituent may be either the same or different at every position.

Combinations of chemical components, e.g., substituents, ring structures, linkers, and/or heteroatoms, envisioned by this disclosure are those that result in the formation of stable or chemically feasible compounds.

In some embodiments, substituents are independently selected from optionally substituted heteroatom and optionally substituted, optionally hetero-, optionally cyclic C₁-C₁₈ hydrocarbyl, particularly wherein the optionally substituted, optionally hetero-, optionally cyclic C₁-C₁₈ hydrocarbyl is optionally-substituted, optionally hetero-, optionally cyclic alkyl, alkenyl or alkynyl, or optionally-substituted, optionally hetero-, aryl; and/or the optionally substituted heteroatom is halogen, optionally substituted hydroxyl (such as alkoxy, aryloxy), optionally substituted acyl (such as formyl, alkanoyl, carbamoyl, carboxyl, amido), optionally substituted amino (such as amino, alkylamino, dialkylamino, amido, sulfamidyl), optionally substituted thiol (such as mercapto, alkylthiol, aryl thiol), optionally substituted sulfinyl or sulfonyl (such as alkylsulfinyl, arylsulfinyl, alkyl sulfonyl, arylsulfonyl), nitro, or cyano.

In some embodiments, substituents are independently selected from: halogen, —R′, —OR′, ═O, ═NR′, ═N—OR′, —NR′R″, —SR′, —SiR′R″R′″, —OC(═O)R′, —C(═O)R′, —CO₂R′, —C(═O)NR′R″, —OC(═O)NR′R″, —NR″C(═O)R′, —NR′—C(═O)NR″R′″, —NR′—SO₂NR″R′″, —NR″CO₂R′, —NH—C(NH₂)═NH, —NR′C(NH₂)═NH, —NH—C(NH₂)═NR′, —S(O)R′, —SO₂R′, —SO₂NR′R″, —NR″SO₂R′, —CN, —NO₂, —N₃, —CH(Ph)₂, perfluoro(C₁-C₄)alkoxy, and perfluoro(C₁-C₄)alkyl, in a number ranging from zero to three, with those groups having zero, one, or two substituents being particularly preferred. R′, R″, and R′″ each independently refer to hydrogen, unsubstituted C₁-C₈alkyl and heteroalkyl, C₁-C₈ alkyl and heteroalkyl substituted with one to three halogens, unsubstituted aryl, aryl substituted with one to three halogens, unsubstituted alkyl, alkoxy, or thioalkoxy groups, or aryl-(C₁-C₄) alkyl groups. When R′ and R″ are attached to the same nitrogen atom, they can be combined with the nitrogen atom to form a 5-, 6- or 7-membered ring. Hence, —NR′R″ includes 1-pyrrolidinyl and 4-morpholinyl. When the aryl group is 1,2,3,4-tetrahydronaphthalenyl, it may be substituted with a substituted or unsubstituted C₃-C₇ spirocycloalkyl group. The C₃-C₇ spirocycloalkyl group may be substituted in the same manner as defined herein for “cycloalkyl.”

In some embodiments, substituents are selected from: halogen, —R′, —OR′, ═O, —NR^{m}Rⁿ, —SR′, —SiR′R″R′″, —OC(═O)R′, —C(═O)R′, —CO₂R′, —C(═O)NR^{m}Rⁿ, —OC(═O)NR^{m}Rⁿ, —NR″C(═O)R′, —NR″CO₂R′, —NR′—SO₂NR″R′″, —S(═O)R′, —SO₂R′, —SO₂NR^{m}Rⁿ, —NR″SO₂R′, —CN, —NO₂, perfluoro C₁-C₄ alkoxy and perfluoro C₁-C₄ alkyl, where R′ and R″ are as defined above.

In some embodiments, substituents are independently selected from substituted or unsubstituted heteroatom, substituted or unsubstituted, 0-3 heteroatom-containing C₁-C₆ alkyl (e.g., C₁-C₃ alkyl or C₁-C₂ alkyl), substituted or unsubstituted, 0-3 heteroatom-containing C₂-C₆ alkenyl (e.g., C₂-C₄ alkenyl), substituted or unsubstituted, 0-3 heteroatom-containing C₂-C₆ alkynyl (e.g., C₂-C₄ alkynyl), or substituted or unsubstituted, 0-3 heteroatom-containing C₅-C₁₄ aryl (e.g., C₅-C₆ aryl), wherein each heteroatom is independently oxygen, phosphorus, sulfur, or nitrogen.

In some embodiments, substituents are independently selected from aldehyde, aldimine, alkanoyloxy, alkoxy, alkoxycarbonyl, alkyloxy, alkyl, alkenyl, alkynyl, amine, azo, halogen, carbamoyl, carbonyl, carboxamido, carboxyl, cyanyl, ester, haloformyl, hydroperoxyl, hydroxyl, imine, isocyanide, isocyante, N-tert-butoxycarbonyl, nitrate, nitrile, nitrite, nitro, nitroso, phosphate, phosphono, sulfide, sulfonyl, sulfo, sulfhydryl, thiol, thiocyanyl, trifluoromethyl, and trifluromethyl ether (OCF₃) groups.

In some embodiments, certain substituents are structurally depicted. For example, when a substituent is attached to a ring structure without a specified position such as in

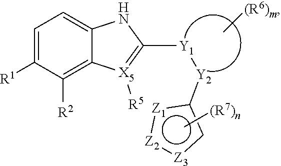

the substituent R⁶, may be attached to any chemically feasible position of Ring B regardless of whether Ring B is a single cyclic or multi-cyclic structure, when m is a positive integer; the substituent R⁷, may be attached to any chemically feasible position of the 5-membered Ring C, when n is a positive integer.

Preferred substituents are disclosed herein and exemplified in the tables, structures, examples, and claims, and may be applied across different compounds of this disclosure. For example, substituents of a given compound may be combinatorically used with other compounds.

It may be advantageous to separate reaction products from one another and/or from starting materials. The desired products of each step or series of steps are separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example, reverse-phase and normal phase; size exclusion; ion exchange; high, medium, and low pressure liquid chromatography methods and apparatus; small scale analytical; simulated moving bed (“SMB”) and preparative thin or thick layer chromatography, as well as techniques of small scale thin layer and flash chromatography. One skilled in the art may apply such techniques to achieve a desired separation.

Non-limiting examples of suitable solvents that may be used in this disclosure include water, methanol (MeOH), ethanol (EtOH), dichloromethane or methylene chloride (CH₂Cl₂), toluene, acetonitrile (MeCN), dimethylformamide (DMF), dimethyl sulfoxide (DMSO), methyl acetate (MeOAc), ethyl acetate (EtOAc), heptanes, isopropyl acetate (IPAc), tert-butyl acetate (t-BuOAc), isopropyl alcohol (IPA), tetrahydrofuran (THF), 2-methyl tetrahydrofuran (2-Me THF), methyl ethyl ketone (MEK), tert-butanol, diethyl ether (Et₂O), methyl-tert-butyl ether (MTBE), 1,4-dioxane, and N-methyl pyrrolidone (NMP).

Non-limiting examples of suitable bases that may be used in this disclosure include 1,8-diazabicyclo[5.4.0]undec-7-ene (DBU), potassium tert-butoxide (KOtBu), potassium carbonate (K₂CO₃), N-methylmorpholine (NMM), triethylamine (Et₃N; TEA), diisopropyl-ethyl amine (i-Pr₂EtN; DIPEA), pyridine, potassium hydroxide (KOH), sodium hydroxide (NaOH), lithium hydroxide (LiOH), and sodium methoxide (NaOMe; NaOCH₃).

The term “subject” refers to an animal including a human.

The term “therapeutically effective amount” refers to the amount of a compound that produces a desired effect for which it is administered (e.g., improvement in a disease or condition, lessening the severity of a disease or condition, and/or reducing progression of a disease or condition, e.g., ALS, Parkinson's disease, multiple sclerosis, traumatic brain injury, diabetic neuropathy, and CIPN). The disease or condition may be caused by or associated with axonal degeneration. The exact amount of a therapeutically effective amount will depend on the purpose of the treatment and will be ascertainable by one skilled in the art using known techniques (see, e.g., Lloyd (1999), The Art, Science and Technology of Pharmaceutical Compounding).

As used herein, the term “treatment” and its cognates refer to slowing or stopping disease progression. “Treatment” and its cognates as used herein include, but are not limited to the following: complete or partial remission, curing a disease or condition or a symptom thereof, lower risk of a disease or condition, e.g., ALS, Parkinson's disease, multiple sclerosis, traumatic brain injury, diabetic neuropathy, and CIPN. The disease or condition may be caused by or associated with axonal degeneration. Improvements in or lessening the severity of any of these symptoms can be assessed according to methods and techniques known in the art.

The terms “about” and “approximately,” when used in connection with a number such as a percentage include the number as specified, and a range of the number (e.g., a range of percentages, for example, a range of ±10% with respect to a specific point value) that is recognized by one of ordinary skill in the art.

### II. Compounds and Compositions

In a 1^{st} embodiment, a compound of this disclosure is a compound of the following structural Formula 1:

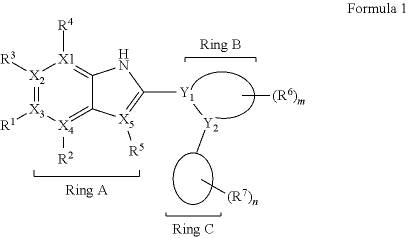

a tautomer thereof, a solvate or stereoisomer of the compound or the tautomer, or a pharmaceutically acceptable salt of the foregoing, wherein:
X₁, X₂, X₃, X₄, and X₅ are each independently C or N (e.g., X₁, X₂, X₃, X₄, and X₅ are all C, X₁, X₂, X₃, X₄, and X₅ are all N; one of X₁, X₂, X₃, X₄, and X₅ is N, and the rest of X₁, X₂, X₃, X₄, and X₅ are C; two of X₁, X₂, X₃, X₄, and X₅ are N, and the rest of X₁, X₂, X₃, X₄, and X₅ are C; three of X₁, X₂, X₃, X₄, and X₅ are N, and the rest of X₁, X₂, X₃, X₄, and X₅ are C; four of X₁, X₂, X₃, X₄, and X₅ are N, and the rest of X₁, X₂, X₃, X₄, and X₅ is C; X₁, X₂, X₃, and X₄ are C, X₅ is N; X₂, X₃, and X₄ are C, X₁ and X₅ are N; X₁, X₃, and X₄ are C, X₂ and X₅ are N; X₁, X₂, and X₄ are C, X₃ and X₅ are N; X₁, X₂, and X₃ are C, X₄ and X₅ are N);
Y₁ is C or N, Y₂ is C or N, and Y₁ and Y₂ are two adjacent ring atoms on Ring B;
Ring B is phenyl, 5- to 6-membered heteroaryl, 3-6 membered cycloalkyl, or 4- to 6-membered heterocyclyl, wherein the 5- to 6-membered heteroaryl or 4- to 7-membered heterocyclyl of Ring B contains 1 to 4 heteroatoms selected from N, O, and S;
Ring C is phenyl, 3- to 10-membered cycloalkyl, 4- to 10-membered heterocyclyl, 5- to 6-membered heteroaryl, or 9- to 10-membered heteroaryl, wherein the 4- to 10-membered heterocyclyl, 5- to 6-membered heteroaryl, or 9- to 10-membered heteroaryl of Ring C contains 1 to 3 heteroatoms selected from N, S, and O;
R¹ is selected from H, halogen, C₁-C₈ alkyl, C₁-C₈ alkenyl, C₁-C₈ alkynyl, —CN, —OH, —COOH, —C(═O)NH₂, —OR^{m}, —S(═O)ₚ(C₁-C₄ alkyl), —NR^{m}Rⁿ, —C(═O)Rⁿ, —C(═O)OR′, —C(═O)NR^{m}Rⁿ, —P(═O)R^{m}Rⁿ, —SF₅,
   5- to 6-membered heteroaryl containing 1 to 3 heteroatoms independently selected from N, O, and S,
   3- to 10-membered heterocyclyl containing 1 to 2 heteroatoms independently selected from N, O, and S, and
   3- to 10-membered cycloalkyl, wherein:
      the C₁-C₈ alkyl, C₁-C₈ alkenyl, or C₁-C₈ alkynyl of R¹ is optionally substituted with 1-3 groups selected from halogen, —OH, —OR^{m}, —CN, —NH₂, —NR^{m}Rⁿ, —C(═O)OCH₃, —O(C₁-C₆ alkyl), —COOH, —C(═O)NH₂, phenyl, 5 to 6-membered heteroaryl, 3 to 6-heterocyclyl, and 3- to 6-membered cycloalkyl (optionally substituted with 1-3 groups selected from OH and halogen),
      the 5- to 6-membered heteroaryl of R¹ is optionally substituted by 1 to 3 groups selected from D, halogen, —OH, —CN, —COOH, —(C₁-C₆ alkyl)OH, —C(═O)O(C₁-C₆ alkyl), ═O, —NH₂, —C(═O)NR^{m}Rⁿ, 5- to 6-membered heteroaryl, —OR^{m}, R^{m}, C₁-C₆ alkyl (optionally substituted with 1-3 groups selected from halogen, —C(═O)NH₂, R^{m}, and OR^{m}),
      the 3- to 10-membered heterocyclyl of R¹ is optionally substituted by 1 to 3 groups selected from D, halogen, —OH, —CN, —COOH, —(C₁-C₆ alkyl)OH, —C(═O)O(C₁-C₆ alkyl), ═O, —NH₂, —C(═O)NR^{m}Rⁿ, 5- to 6-membered heteroaryl, —OR″ ¹, R^{m}, C₁-C₆ alkyl (optionally substituted with 1-3 groups selected from halogen, —C(═O)NH₂, R^{m}, and OR^{m}), and
      the 3- to 10-membered cycloalkyl of R¹ is optionally substituted by 1 to 3 groups selected from D, halogen, —OH, —CN, —COOH, —(C₁-C₆ alkyl)OH, —C(═O)O(C₁-C₆ alkyl), ═O, —NH₂, —C(═O)NR^{m}Rⁿ, 5- to 6-membered heteroaryl, —OR^{m}, R^{m}, C₁-C₆ alkyl (optionally substituted with 1-3 groups selected from halogen, —C(═O)NH₂, R^{m}, and OR^{m}), and
   wherein R^{m} and Rⁿ, for each occurrence, are each independently selected from H, C₁-C₆alkyl, —S(═O)ₚ(C₁-C₄ alkyl), phenyl, 3- to 8-membered cycloalkyl, 4- to 6-membered heterocyclyl, and 5- to 6-membered heteroaryl, wherein the C₁-C₆ alkyl of R^{m} is optionally substituted with 1-3 groups selected from D, —C(═O)NH₂, —OH, —OMe, —S(═O)₂CH₃, and halogen;
R² is selected from H, halogen, C₁-C₆ alkyl, C₁-C₆ alkenyl, —OH, —O(C₁-C₆ alkyl), —O(C₁-C₆ alkyl)O(C₁-C₆ alkyl), —C(═O)NH₂, —S(═O)ₚ(C₁-C₄ alkyl), —CN, 3- to 6-membered cycloalkyl, phenyl, 5- to 6-membered heteroaryl, and 4- to 10-membered heterocyclyl (containing 1 to 3 heteroatoms independently selected from S, O, and N), wherein:
   the C₁-C₆ alkyl or C₁-C₆ alkenyl of R² is optionally substituted with 1-3 groups selected from halogen, CN, and —C(═O)O(C₁-C₆ alkyl),
   the 3- to 5-membered cycloalkyl of R² is optionally substituted with 1-3 groups selected from OH, CN, and halogen,
   the C₁-C₆ alkyl of the —O(C₁-C₆ alkyl) of R² is optionally substituted with 1-3 groups selected from halogen and CN, and
   the 3- to 10-membered heterocyclyl of R² is optionally substituted with 1-3 groups selected from OH, CN, and halogen; or
R¹ and R² join to form

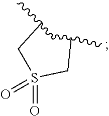

R³ and R⁴ are each independently selected from H, halogen, C₁-C₆ alkyl (optionally substituted with 1-3 groups selected from OH and halogen), and —O(C₁-C₆ alkyl);
R⁵ is selected from absent, H, —CN, halogen, —C(═O)NH₂, —S(═O)ₚ(C₁-C₄ alkyl), —OR^{p}, phenyl, 5- to 6-membered heteroaryl, 4- to 6-membered heterocyclyl, 3- to 8-membered cycloalkyl, and C₁-C₆ alkyl, wherein:
   the C₁-C₆ alkyl of R⁵ is optionally substituted with 1 to 3 groups selected from OH, —NHR^{p}, —OR^{p}, and —S(═O)ₚ(C₁-C₄ alkyl),
   the 4- to 6-membered heterocyclyl of R⁵ is optionally substituted with 1 to 3 groups selected from C₁-C₃ alkyl, CN, halogen, and ═O,
   the 3- to 8-membered cycloalkyl of R⁵ is optionally substituted with 1 to 3 groups selected from C₁-C₃ alkyl, CN, and halogen, wherein:
   R^{p} is selected from C₁-C₆ alkyl, 3- to 6-membered cycloalkyl, and 5- to 6-membered heteroaryl, wherein the C₁-C₆ alkyl, 3- to 6-membered cycloalkyl, or 5- to 6-membered heteroaryl of R^{p} is optionally substituted with 1 to 3 groups selected from CN, OH, and halogen;
R⁶, for each occurrence, is independently selected from D, halogen, —CN, ═O, —OR^{s}, —SH, —S(C₁-C₄ alkyl), —S(═O)ₚR^{t}, —C(═O)NR^{t}R^{o}, —NR^{t}R^{o}, 4- to 6-membered heterocyclyl, and C₁-C₆ alkyl, wherein:
   the C₁-C₆ alkyl of R⁶ is optionally substituted with 1 to 3 groups selected from halogen, —OR′, ═O, —S(═O)ₚR^{t}, —NHS(═O)ₚR^{t}, —S(═O)(═NH)R^{t},

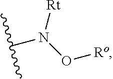

NHS(═O)ₚ(C₁-C₄ alkyl), —CN, —C(═O)NR^{t}R^{o}, —NR^{t}R^{o}, halogen, 5- to 6-membered heteroaryl, 3- to 6-membered cycloalkyl (optionally substituted with 1 to 3 groups selected from halogen, OH, and R^{t}), and 4- to 10-membered heterocyclyl optionally substituted with 1 to 3 groups selected from halogen, OH, and R^{t}, wherein: the 4- to 8-membered heterocyclyl of the C₁-C₆ alkyl of R⁶ is optionally substituted with 1 to 3 groups selected from halogen, OH, C₁-C₃ alkyl, and ═O;
R^{s} is selected from H, C₁-C₆ alkyl, 4- to 6-membered heterocyclyl, and 3- to 6-membered cycloalkyl, wherein:
   the C₁-C₆ alkyl of R^{s} is optionally substituted with 1-3 groups selected from —OH, —OMe, and halogen and
   the 3- to 6-membered cycloalkyl of R^{s} is optionally substituted with —OH or —OMe;
R^{t} and R^{o}, for each occurrence, are each independently selected from H, C₁-C₆ alkyl, 5- to 6-membered heteroaryl, 4- to 6-membered heterocyclyl, and 3- to 5-membered cycloalkyl, wherein the C₁-C₆ alkyl of R^{t} and R^{o} is optionally substituted with 1-3 groups selected from D, halogen, —OH, CN, C(═O)NH₂, —O(C₁-C₃ alkyl), and —S(═O)₂CH₃;
R⁷, for each occurrence, is independently selected from D, halogen, —OR^{a}, —CN, —CONH₂, —C(═O)NR^{b}R^{c}, NR^{b}R^{c}, —C(═O)OR^{b}, ═O, ═S, —P(═O)₂R^{b}R^{c}, —S(═O)ₚ(C₁-C₄ alkyl), —O(C₁-C₆ alkyl), C₁-C₆ alkyl, C₁-C₆ alkenyl, C₁-C₆ alkynyl, 3- to 6-membered cycloalkyl, 4- to 6-membered heterocyclyl, and 5- to 6-membered heteroaryl, wherein:
   the C₁-C₆ alkyl, C₁-C₆ alkenyl, or C₁-C₆ alkynyl of R⁷ is optionally substituted with 1 to 3 groups selected from halogen, —OH, CN, —S(═O)ₚ(C₁-C₄ alkyl), —C(═O)₂NH₂, and 3- to 6-membered heterocyclyl,
   the 4- to 6-membered heterocyclyl of R⁷ is optionally substituted with 1 to 3 groups selected from ═O, halogen, and R^{b},
   R^{a} is selected from H, C₁-C₈ alkyl, 3- to 6-membered cycloalkyl, 4- to 6-membered heterocyclyl, phenyl, and 5- to 6-membered heteroaryl, wherein the C₁-C₈ alkyl of R^{a} is optionally substituted with 1 to 4 groups selected from D, halogen, OH, CN, —S(═O)ₚ(C₁-C₄ alkyl), —C(═O)NH₂, 3- to 6-membered cycloalkyl, 4- to 6-membered heterocyclyl, and 5- to 6-membered heteroaryl,
   R^{b} and R^{c}, for each occurrence, are each independently selected from H, C₁-C₈ alkyl, 4- to 6-membered heterocyclyl, phenyl, 5- to 6-membered heteroaryl, and 3- to 6-membered cycloalkyl, wherein the C₁-C₈ alkyl of R^{b} and R^{o} is optionally substituted with 1 to 3 groups selected from D, halogen, OH, —C(═O)NH₂, CN, —OCH₃, and —S(═O)₂CH₃;
m is an integer selected from 0, 1, and 2;
n is an integer selected from 0, 1, 2, 3, and 4; and
p is an integer selected from 0, 1, and 2,
wherein, in each occurrence, the C₁ to C₈ alkyl can be independently C₁ alkyl, C₂ alkyl, C₃ alkyl, C₄ alkyl, C₅ alkyl, C₆ alkyl, C₇ alkyl, or C₈ alkyl; the C₁ to C₆ alkyl can be C₁ alkyl, C₂ alkyl, C₃ alkyl, C₄ alkyl, C₅ alkyl, or C₆ alkyl; the C₁ to C₄ alkyl can be C₁ alkyl, C₂ alkyl, C₃ alkyl, or C₄ alkyl; 3- to 5-membered means 3-, 4-, or 5-membered; 3- to 6-membered means 3-, 4-, 5-, or 6-membered; 3- to 8-membered means 3-, 4-, 5-, 6, 7, or 8-membered; 3- to 10-membered means 3-, 4-, 5-, 6, 7, 8-, 9-, or 10-membered; 4- to 6-membered means 4-, 5-, or 6-membered; 4- to 7-membered means 4-, 5-, 6, or 7-membered; 4- to 8-membered means 4-, 5-, 6, 7, or 8-membered; 5- to 7-membered means 5-, 6-, or 7-membered; 1-3 or 1 to 3 groups means 1 group, 2 groups, or 3 groups; 1 to 3 or 1-3 heteroatoms means 1 heteroatom, 2 heteroatoms, or 3 heteroatoms; and 1 to 4 or 1-4 heteroatoms means 1 heteroatom, 2 heteroatoms, 3 heteroatoms, or 4 heteroatoms. The heteroatoms can be at any chemically feasible positions of a cyclic structure.

Combinations of chemical components (such as substituents, ring structures, or heteroatoms) as disclosed herein are those that result in the formation of stable or chemically feasible compounds. For abbreviation or according to common practice, certain hydrogen atoms attached to a certain atom (e.g., a carbon atom C or a nitrogen atom N) are not specifically spelled out in a chemical structure, formula, or notation; hydrogen atoms are deemed to be present to the extent the valences of the certain atom (e.g., C or N) are completed.

In a 2^{nd} embodiment, in a compound, tautomer, a solvate or stereoisomer of the compound or the tautomer, or pharmaceutically acceptable salt of this disclosure, X₁, X₂, X₃, and X₄ are C; and all other variables not specifically defined herein are as defined in the preceding embodiment.

In a 3^{rd} embodiment, a compound of the disclosure is a compound of the following structural Formula 2:

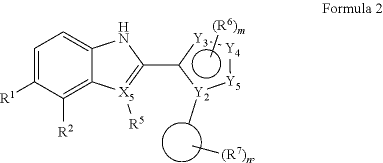

a tautomer thereof, a solvate or stereoisomer of the compound or the tautomer, or a pharmaceutically acceptable salt of the foregoing, wherein Y₂, Y₃, and Y₄ are each independently selected from N and C, at least one of Y₂, Y₃, and Y₄ is N, and Y₅ is selected from S and C; and all other variables not specifically defined herein are as defined in any one of the suitable preceding embodiments.

In a 4^{th} embodiment, a compound of the disclosure is a compound of the following structural Formula 3-1, 3-2, or 3-3:

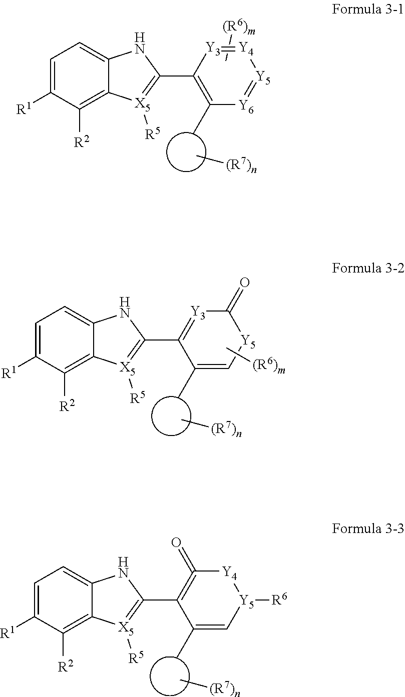

a tautomer thereof, a solvate or stereoisomer of the compound or the tautomer, or a pharmaceutically acceptable salt of the foregoing, wherein Y₃, Y₄, Y₅, and Y₆ are each independently selected from N and C and at least one of Y₃, Y₄, Y₅, and Y₆ is N; and all other variables not specifically defined herein are as defined in any one of the suitable preceding embodiments.

In a 5^{th} embodiment, a compound of the disclosure is a compound of the following structural Formula 4:

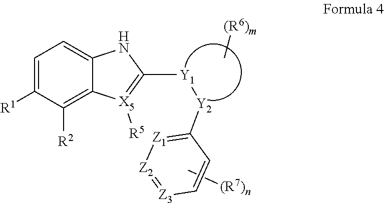

a tautomer thereof, a solvate or stereoisomer of the compound or the tautomer, or a pharmaceutically acceptable salt of the foregoing, wherein Z₁, Z₂, and Z₃ are each independently selected from N and C; and all other variables not specifically defined herein are as defined in any one of the suitable preceding embodiments.

In a 6^{th} embodiment, a compound of the disclosure is a compound of the following structural Formula 5:

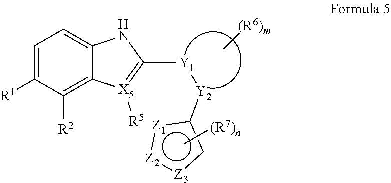

a tautomer thereof, a solvate or stereoisomer of the compound or the tautomer, or a pharmaceutically acceptable salt of the foregoing, wherein Z₁, Z₂, and Z₃ are each independently selected from N, S, and C; and all other variables not specifically defined herein are as defined in any one of the suitable preceding embodiments.

In a 7^{th} embodiment, a compound of the disclosure is a compound of the following structural Formula 6-1 or 6-2:

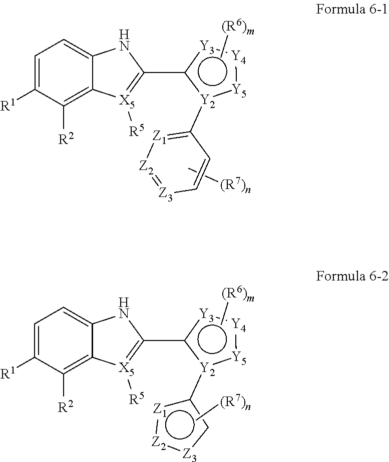

a tautomer thereof, a solvate or stereoisomer of the compound or the tautomer, or a pharmaceutically acceptable salt of the foregoing, wherein Y₃ and Y₄ and Y₅ are each independently selected from N, S, O, and C, Y₂ is selected from N and C, the Z₁, Z₂, and Z₃ of Formula 6-1 are each independently selected from N and C, and the Z₁, Z₂, and Z₃ of Formula 6-2 are each independently selected from N, S, and C; and all other variables not specifically defined herein are as defined in any one of the suitable preceding embodiments.

In a 8^{th} embodiment, a compound of the disclosure is a compound of the following structural Formula 7-1 or 7-2:

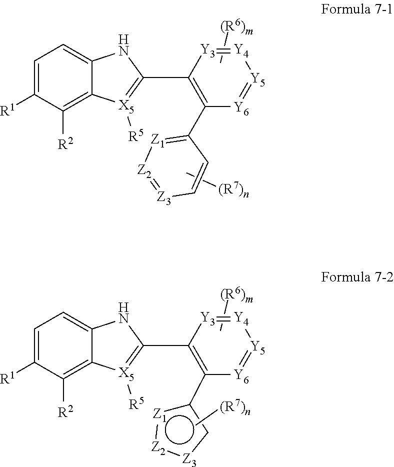

a tautomer thereof, a solvate or stereoisomer of the compound or the tautomer, or a pharmaceutically acceptable salt of the foregoing, wherein Y₃, Y₄, Y₅, and Y₆ are each independently selected from N and C and at least one of Y₃, Y₄, Y₅, and Y₆ is N, the Z₁, Z₂, and Z₃ of Formula 7-1 are each independently selected from N and C, and the Z₁, Z₂, and Z₃ of Formula 7-2 are each independently selected from N, S, and C; and all other variables not specifically defined herein are as defined in any one of the suitable preceding embodiments.

In a 9^{th} embodiment, a compound of the disclosure is a compound of the following structural Formula 8-1, 8-2, 8-3, 8-4, 8-5, or 8-6:

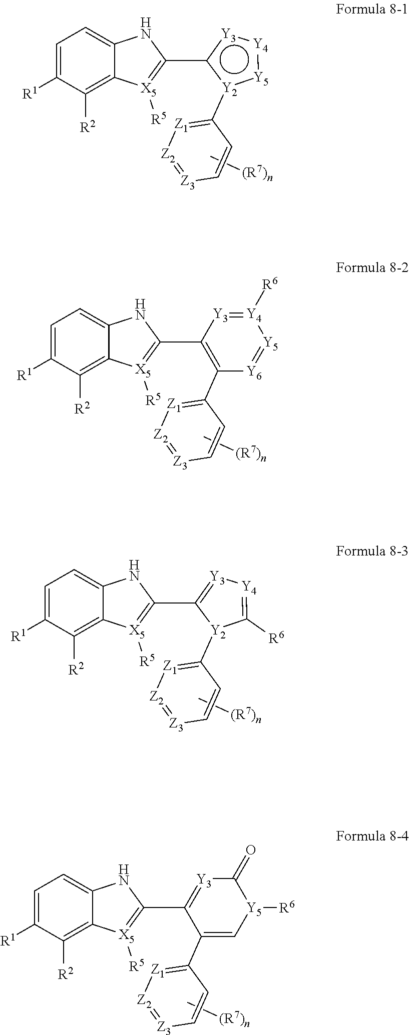

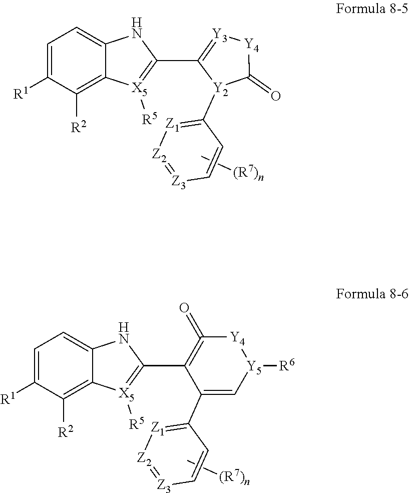

a tautomer thereof, a solvate or stereoisomer of the compound or the tautomer, or a pharmaceutically acceptable salt of the foregoing, wherein:
Y₃, Y₄, Y₅, and Y₆ of Formula 8-2, Formula 8-4, and Formula 8-6 are each independently selected from N and C and at least one of Y₃, Y₄, Y₅, and Y₆ is N,
Y₂, Y₃, and Y₄ of Formula 8-1, Formula 8-3, and Formula 8-5 are each independently selected from N and C, at least one of Y₂, Y₃, and Y₄ is N, Y₅ of Formula 8-1 is selected from S and C;
Z₁, Z₂, and Z₃ are each independently selected from N and C; and all other variables not specifically defined herein are as defined in any one of the suitable preceding embodiments.

In a 10^{th} embodiment, a compound of the disclosure is a compound of the following structural Formula 9-1, 9-2, 9-3, or 9-4:

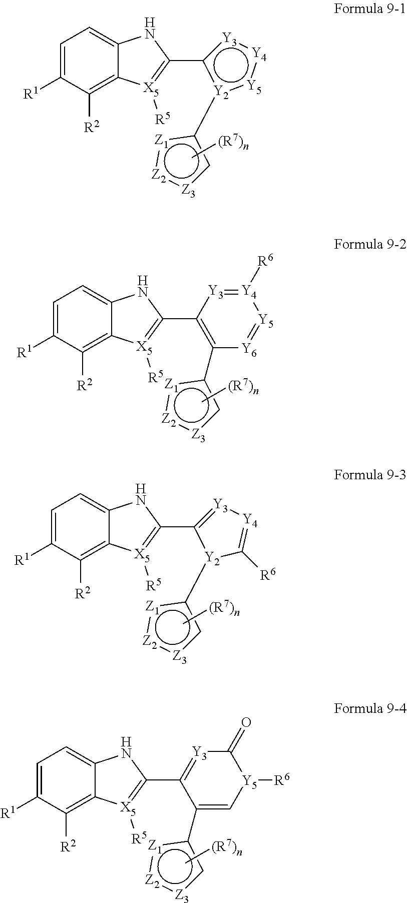

a tautomer thereof, a solvate or stereoisomer of the compound or the tautomer, or a pharmaceutically acceptable salt of the foregoing, wherein:
Y₃, Y₄, Y₅, and Y₆ of Formula 9-2 and Formula 9-4 are each independently selected from N and C and at least one of Y₃, Y₄, Y₅, and Y₆ is N,
Y₂, Y₃, and Y₄ of Formula 9-1 and Formula 9-3 are each independently selected from N and C and at least one of Y₂, Y₃, and Y₄ is N, Y₅ of Formula 9-1 is selected from S and C;
Z₁ and Z₂ are each independently selected from N and S, Z₃ is selected from N and C, and at least one of Z₁, Z₂, and Z₃ is a heteroatom; and all other variables not specifically defined herein are as defined in any one of the suitable preceding embodiments.

In a 11^{th} embodiment, a compound of the disclosure is a compound of the following structural Formula 10-1, 10-2, 10-3, 10-4, 10-5, or 10-6:

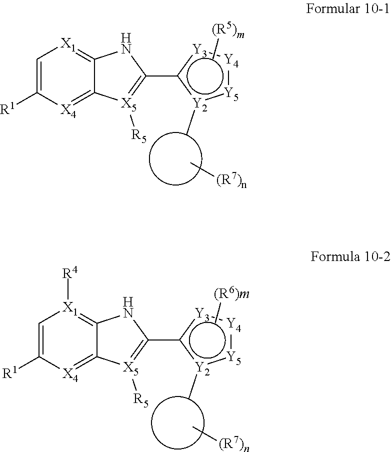

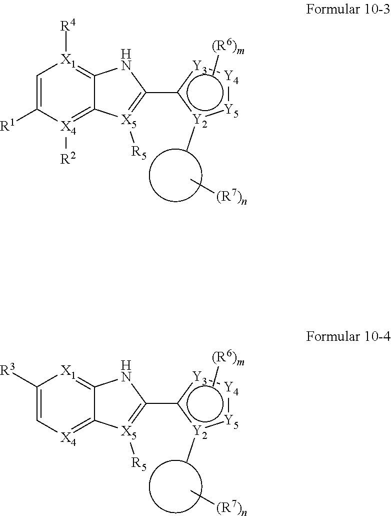

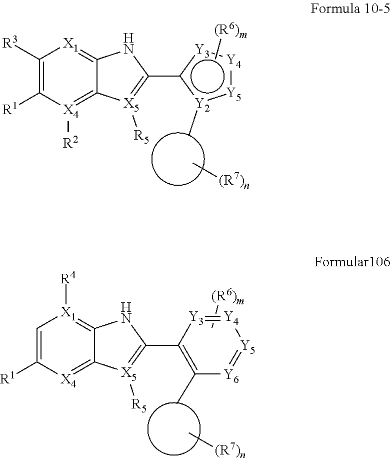

a tautomer thereof, a solvate or stereoisomer of the compound or the tautomer, or a pharmaceutically acceptable salt of the foregoing, wherein:
Y₂, Y₃, and Y₄ of Formula 10-1 to 10-5 are each independently selected from N and C, Y₅ is selected from N, S, and C, and at least one of Y₂, Y₃, Y₄, and Y₅ is a heteroatom;
Y₃, Y₄, Y₅, and Y₆ of Formula 10-6 are each independently selected from N and C, and at least one of Y₃, Y₄, Y₅, and Y₆ is a heteroatom; and all other variables not specifically defined herein are as defined in any one of the suitable preceding embodiments.

In a 12^{th} embodiment, a compound of the disclosure is a compound of the following structural Formula 11-1, 11-2, 11-3, 11-4, 11-5, 11-6, or 11-7:

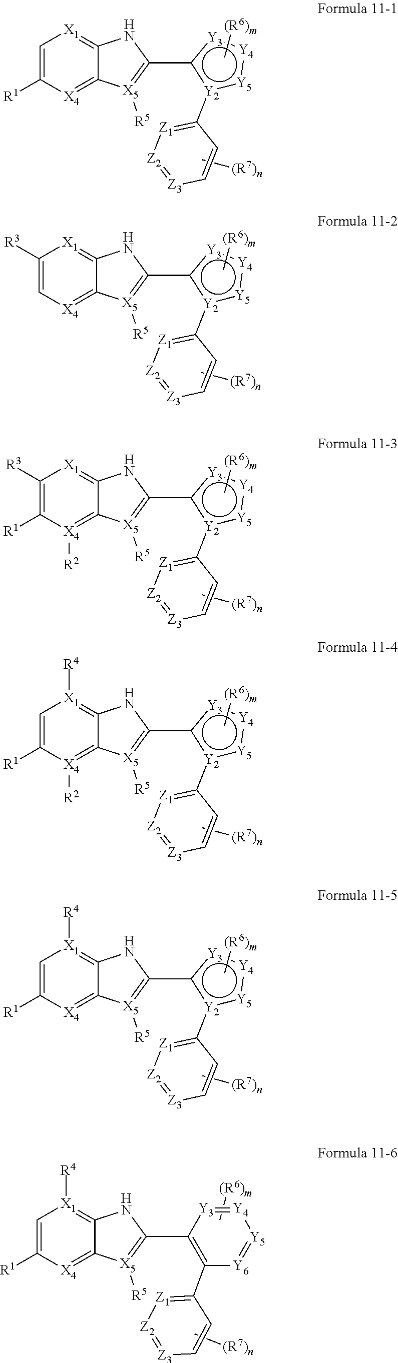

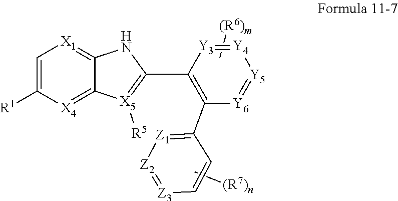

a tautomer thereof, a solvate or stereoisomer of the compound or the tautomer, or a pharmaceutically acceptable salt of the foregoing, wherein:
in Formula 11-1 to 11-5, Y₂, Y₃, and Y₄ are each independently selected from N and C, Y₅ is selected from N, S, and C, at least one of Y₂, Y₃, Y₄, and Y₅ is a heteroatom, Z₁, Z₂, and Z₃ are each independently selected from N and C;
in Formula 11-6 to 11-7, Y₃, Y₄, Y₅, and Y₆ are each independently selected from N and C, at least one of Y₃, Y₄, Y₅, and Y₆ is a heteroatom, Z₁, Z₂, and Z₃ are each independently selected from N and C; all other variables not specifically defined herein are as defined in any one of the suitable preceding embodiments.

In a 13^{th} embodiment, a compound of the disclosure is a compound of the following structural Formula 12-1, 12-2, 12-3, 12-4, 12-5, 12-6, 12-7, or 12-8:

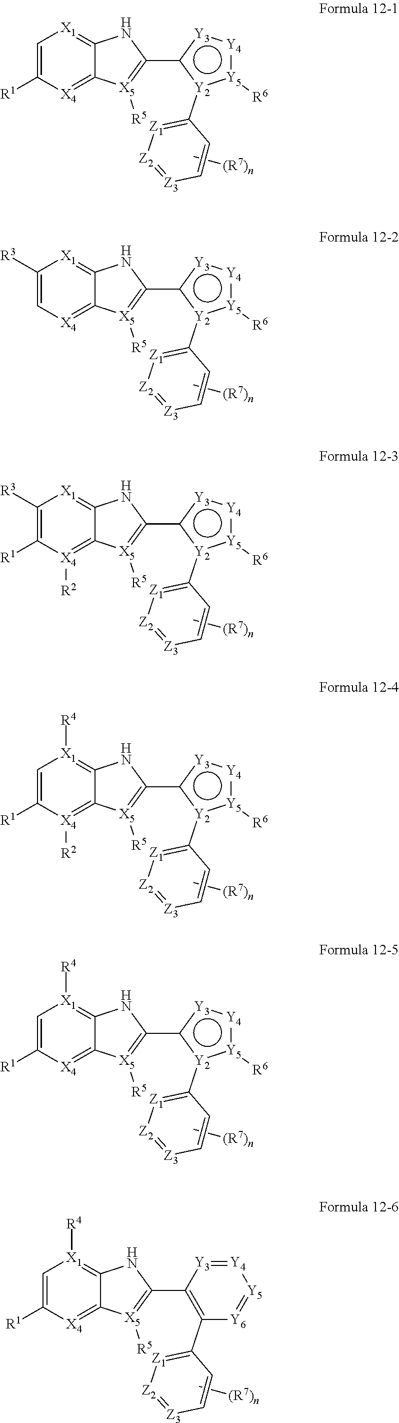

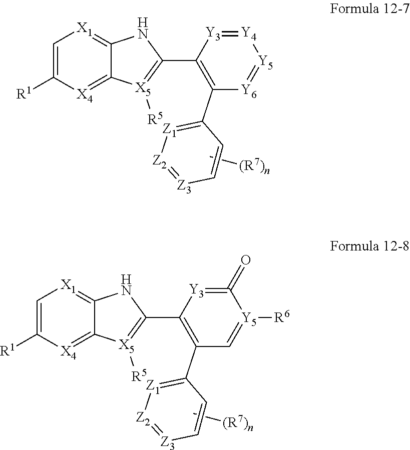

a tautomer thereof, a solvate or stereoisomer of the compound or the tautomer, or a pharmaceutically acceptable salt of the foregoing, wherein:
in Formula 12-1 to 12-5, Y₂ and Y₅ are each independently selected from N and C, Y₃ and Y₄ are each independently selected from N, S, and C, at least one of Y₂, Y₃, Y₄, and Y₅ is a heteroatom, and Z₁, Z₂, and Z₃ are each independently selected from N and C;
in Formula 12-6 to 12-8, Y₃, Y₄, Y₅, and Y₆ are each independently selected from N and C, at least one of Y₃, Y₄, Y₅, and Y₆ is a heteroatom, and Z₁, Z₂, and Z₃ are each independently selected from N and C; and all other variables not specifically defined herein are as defined in any one of the suitable preceding embodiments.

In a 14^{th} embodiment, in a compound, tautomer, a solvate or stereoisomer of the compound or the tautomer, or pharmaceutically acceptable salt of this disclosure,

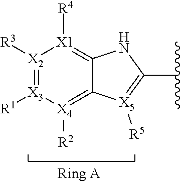

is selected from:

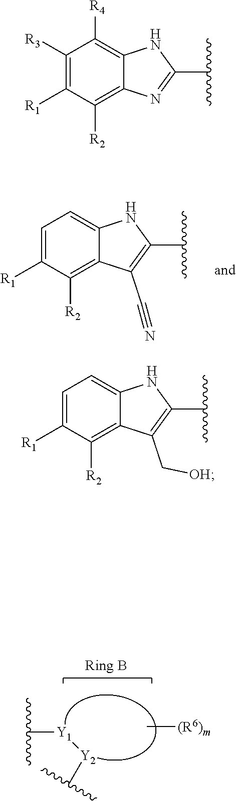

is selected from

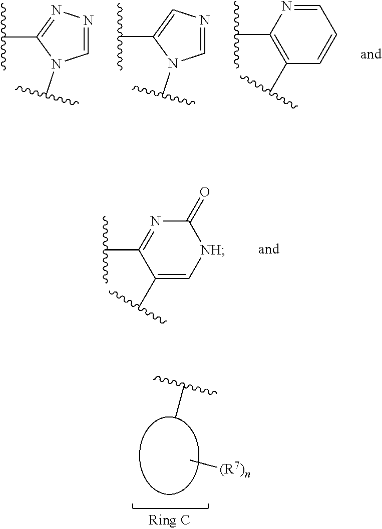

is selected from:

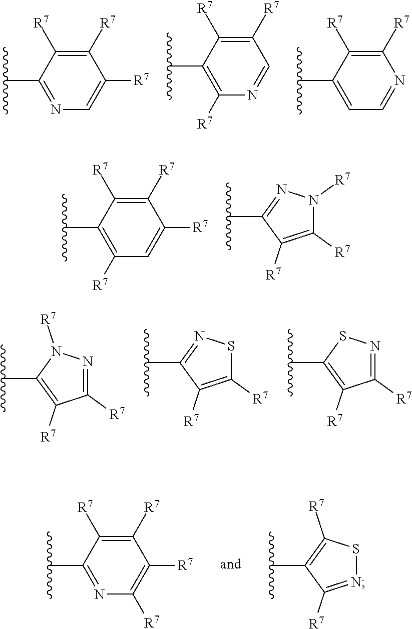

and all other variables not specifically defined herein are as defined in any one of the suitable preceding embodiments.

In a 15^{th} embodiment, in a compound, tautomer, a solvate or stereoisomer of the compound or the tautomer, or pharmaceutically acceptable salt of this disclosure,

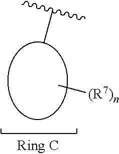

is selected from:

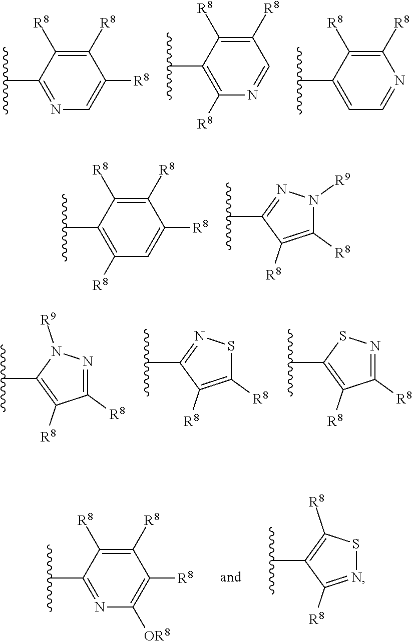

wherein:
R⁸ is selected from: H, F, Cl, Me, CHF₂, CF₃, CN, SO₂Me, SMe, CH₂CF₃, CH₂SO₂Me,

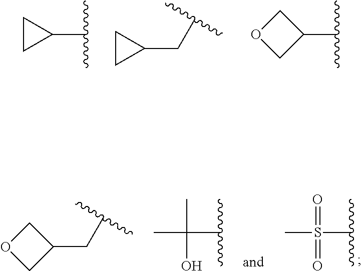

and
R⁹ is selected from: Me, CF₃, CHF₂, CH₂CF₃, Acetyl (—C(═O)CH3), SO₂Me,

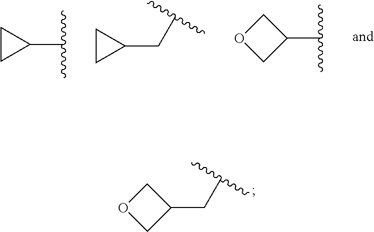

and all other variables not specifically defined herein are as defined in any one of the suitable preceding embodiments.

In a 16^{th} embodiment, in a compound, tautomer, a solvate or stereoisomer of the compound or the tautomer, or pharmaceutically acceptable salt of this disclosure,

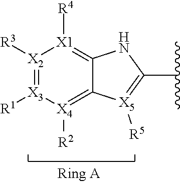

is selected from:

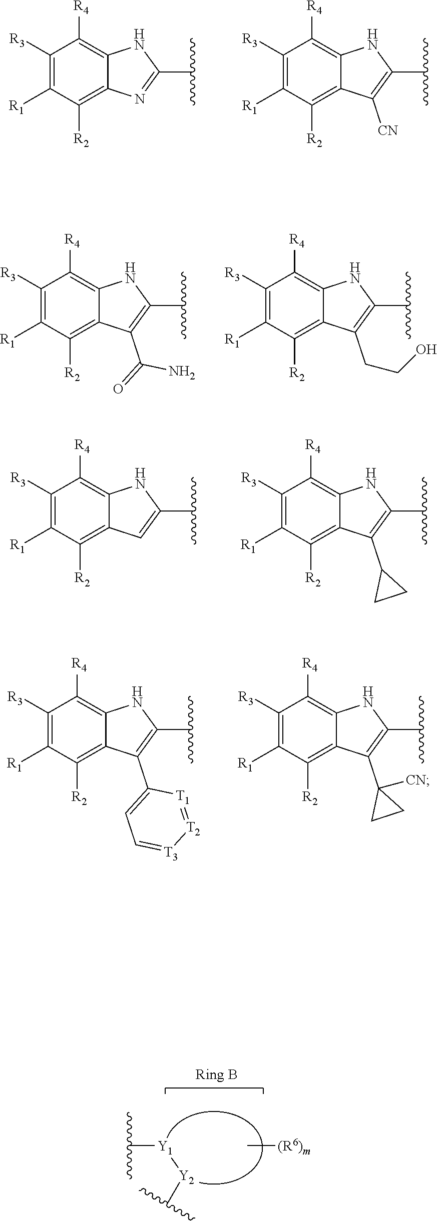

is selected from:

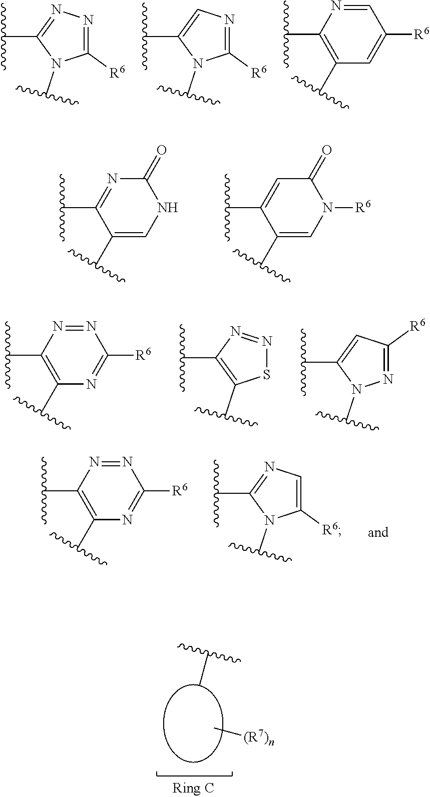

is selected from:

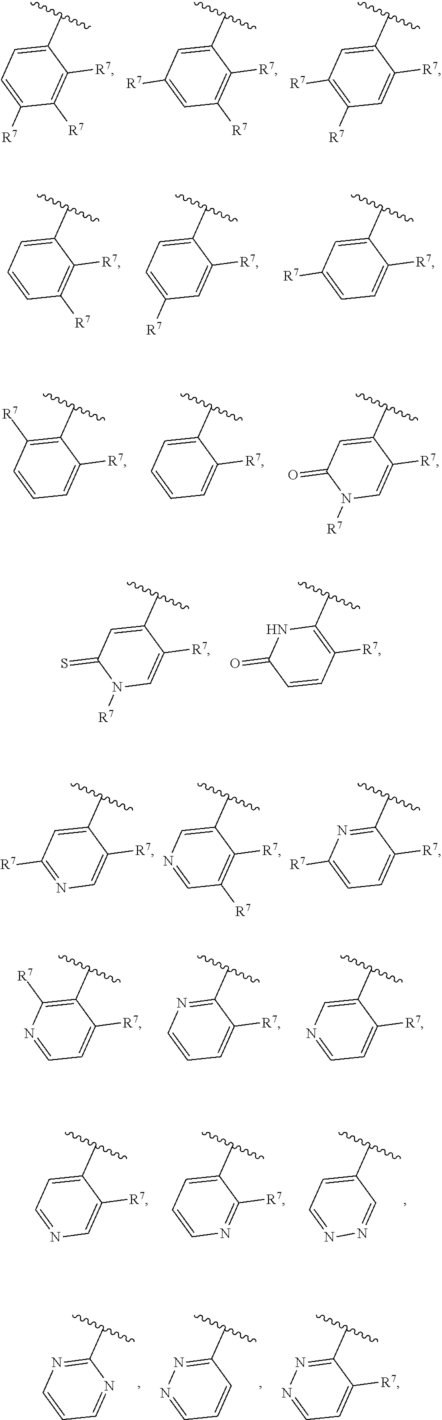

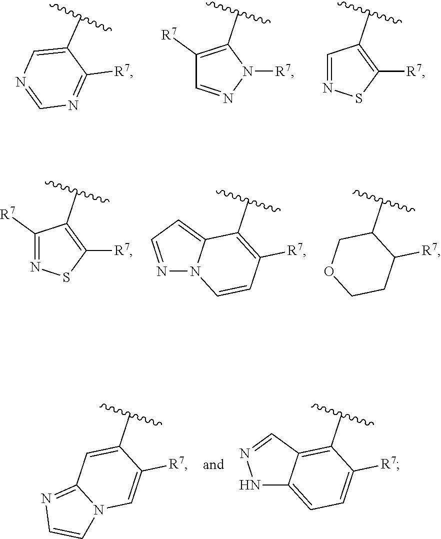

wherein T₁, T₂, and T₃ are each independently selected from N and C; and all other variables not specifically defined herein are as defined in any one of the suitable preceding embodiments.

In a 17^{th} embodiment, in a compound, tautomer, a solvate or stereoisomer of the compound or the tautomer, or pharmaceutically acceptable salt of this disclosure, Ring A is selected from:

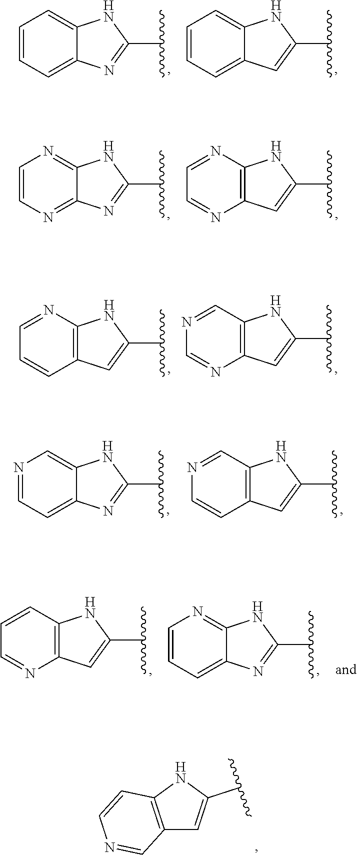

wherein Ring A is substituted with R¹, R², R³, R⁴, and R⁵; and all other variables not specifically defined herein are as defined in any one of the suitable preceding embodiments.

In a 18^{th} embodiment, in a compound, tautomer, a solvate or stereoisomer of the compound or the tautomer, or pharmaceutically acceptable salt of this disclosure,

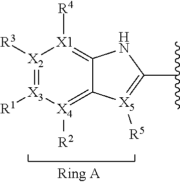

is selected from:

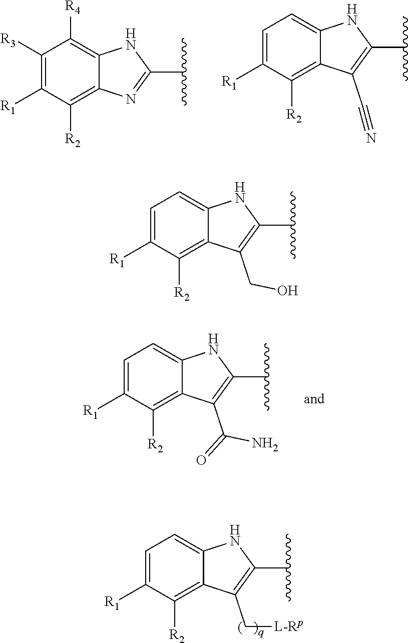

wherein L is —NH— or —O—, q is 1, 2, or 3, and R^{p} is selected from C₁-C₄ alkyl, 3- to 6-membered cycloalkyl, and 5- to 7-membered heteroaryl; and all other variables not specifically defined herein are as defined in any one of the suitable preceding embodiments.

In a 19^{th} embodiment, in a compound, tautomer, a solvate or stereoisomer of the compound or the tautomer, or pharmaceutically acceptable salt of this disclosure,

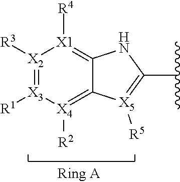

is selected from:

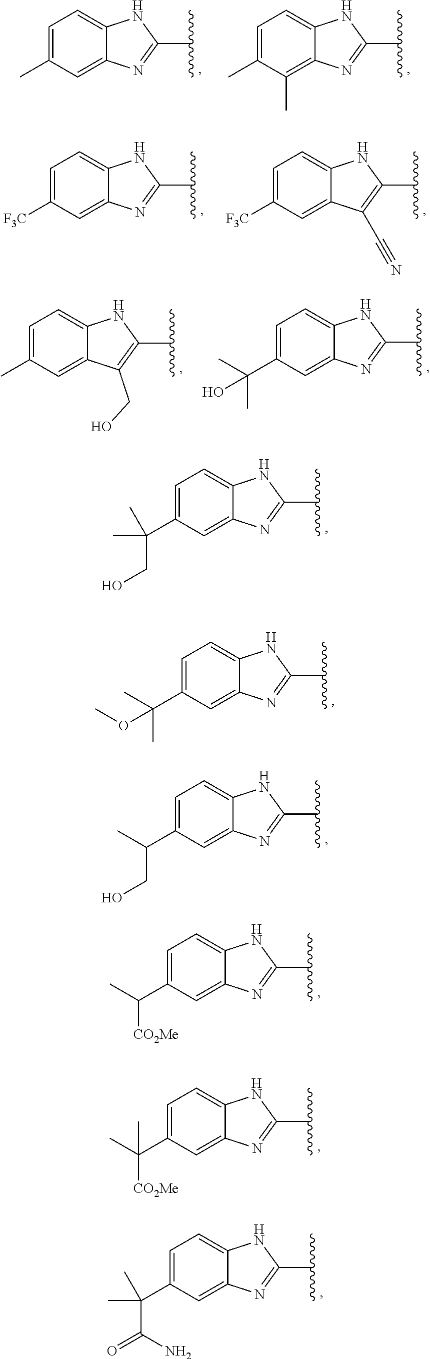

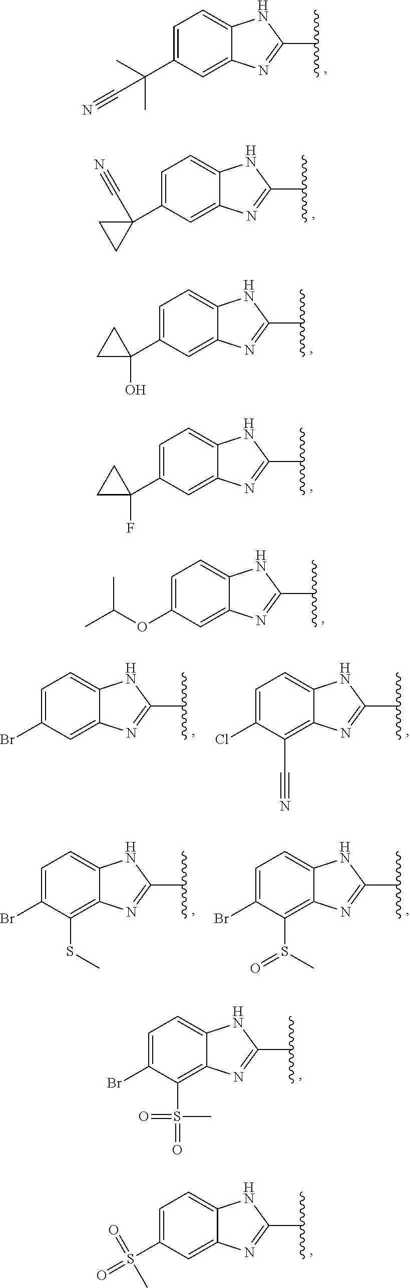

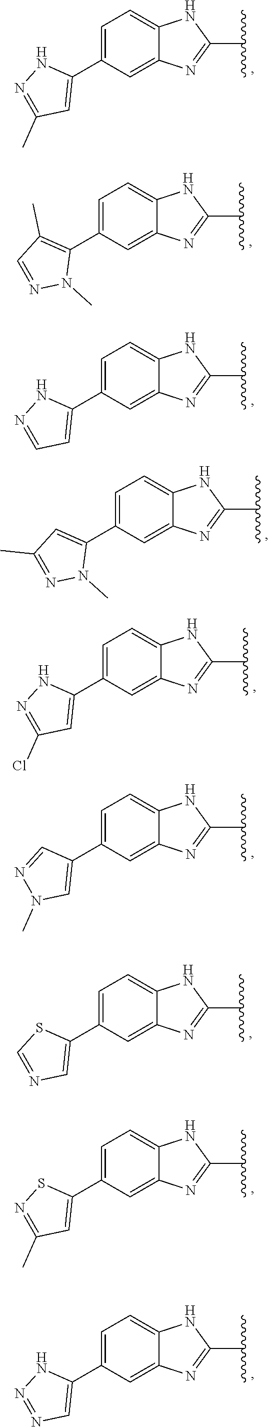

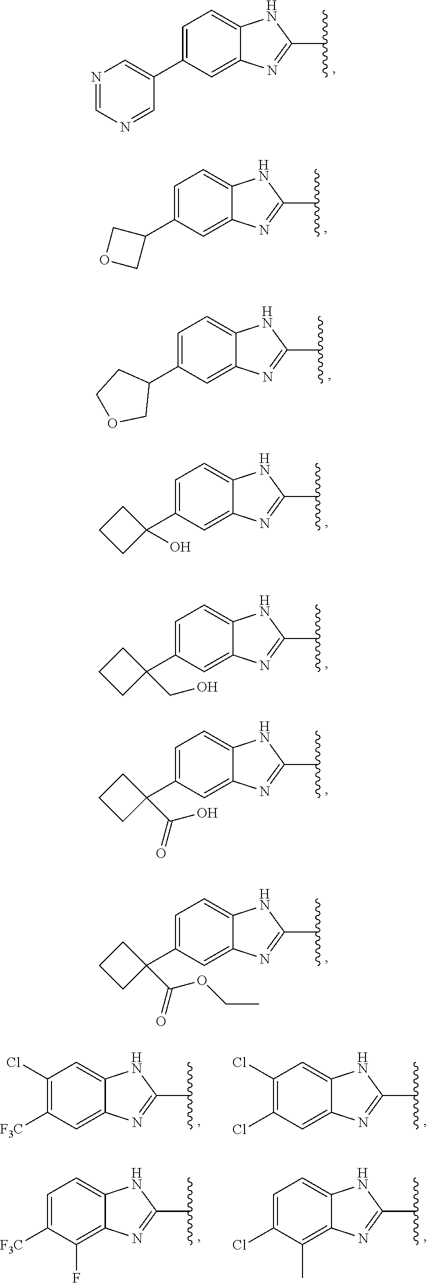

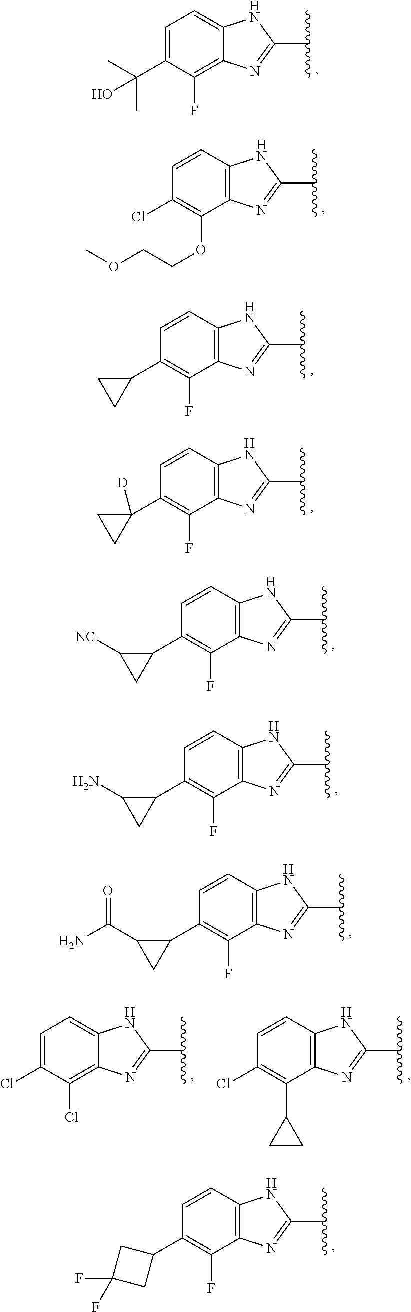

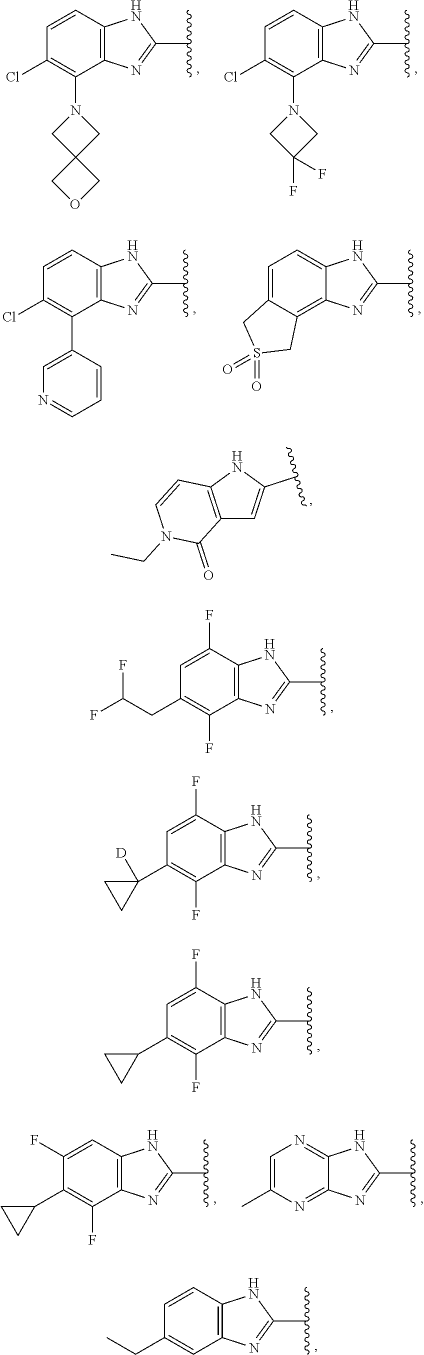

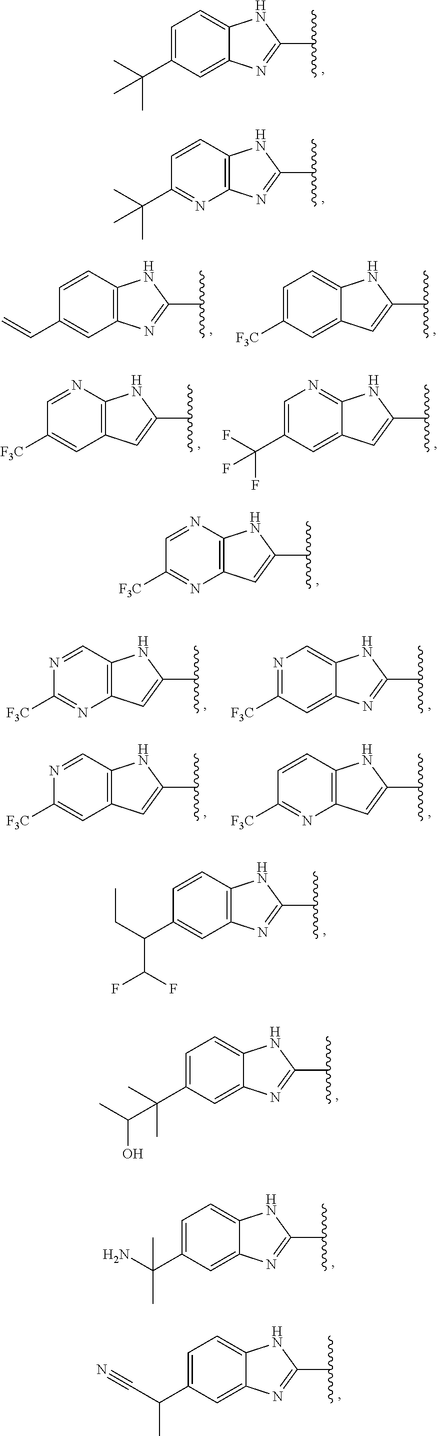

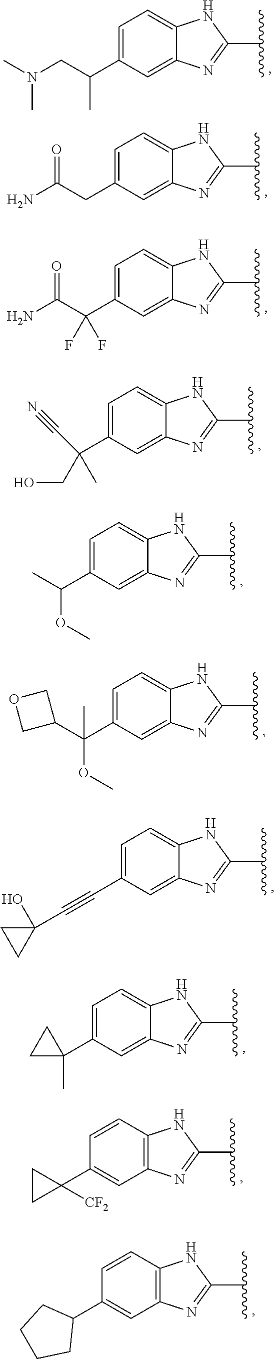

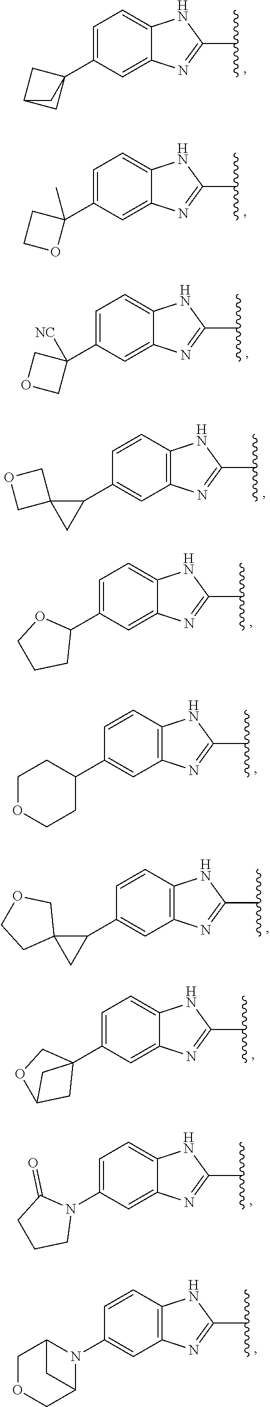

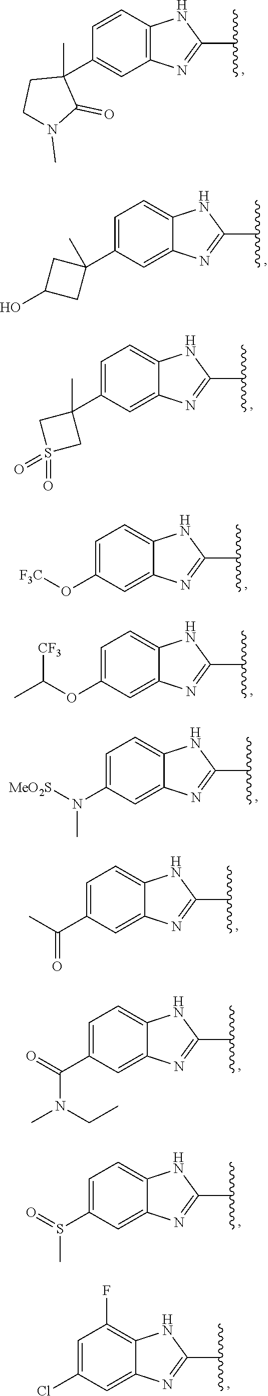

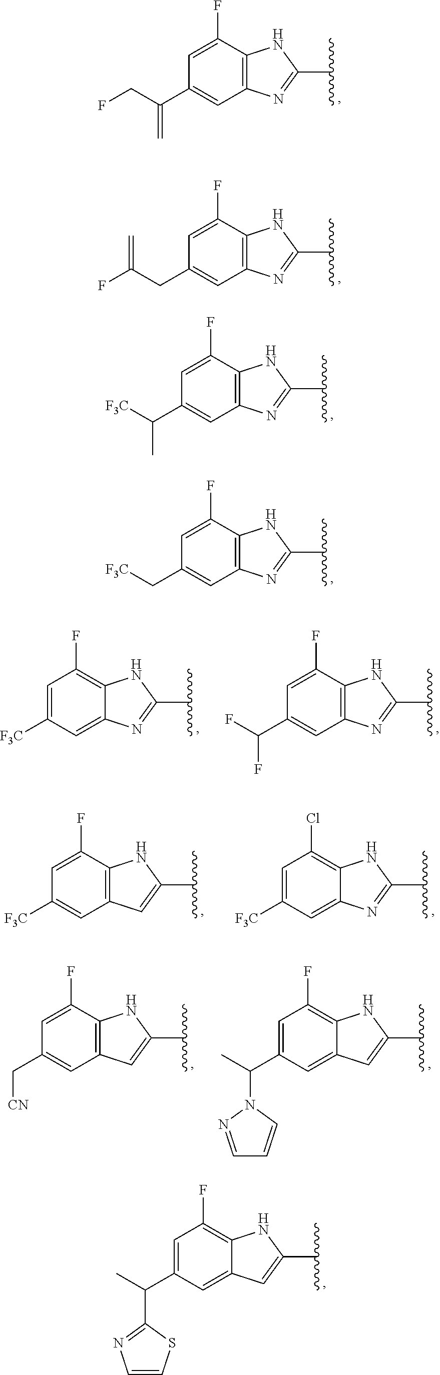

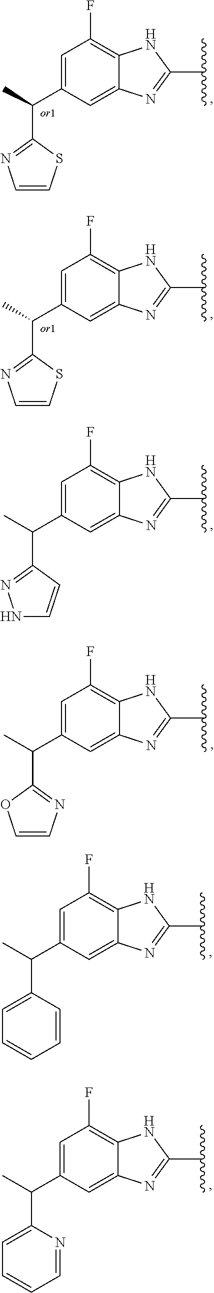

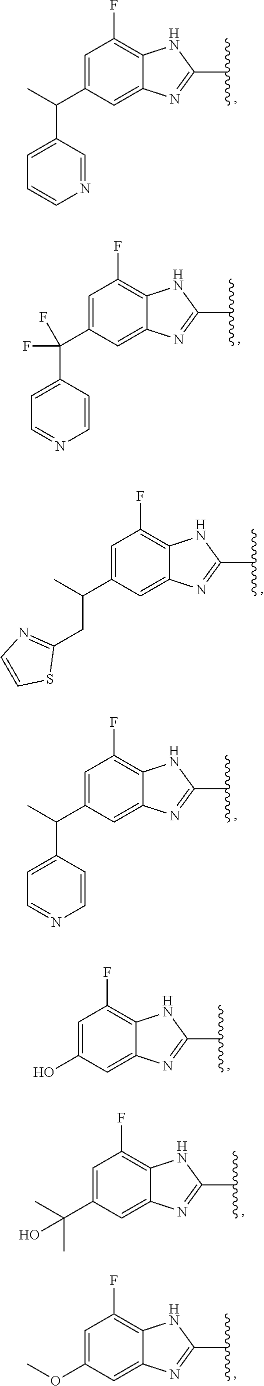

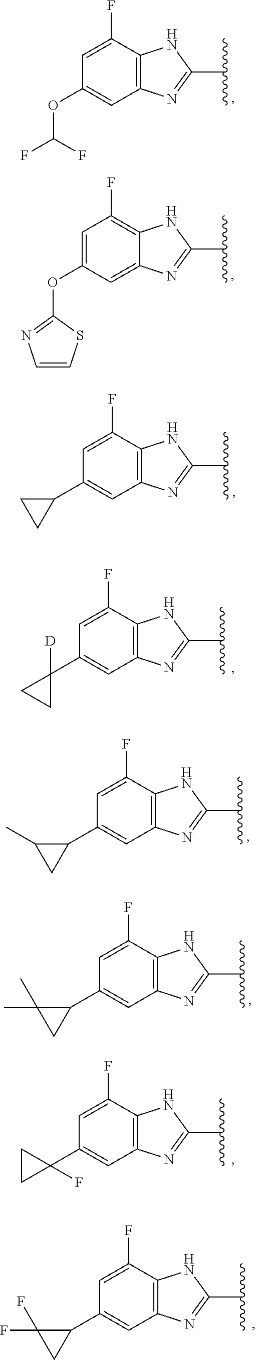

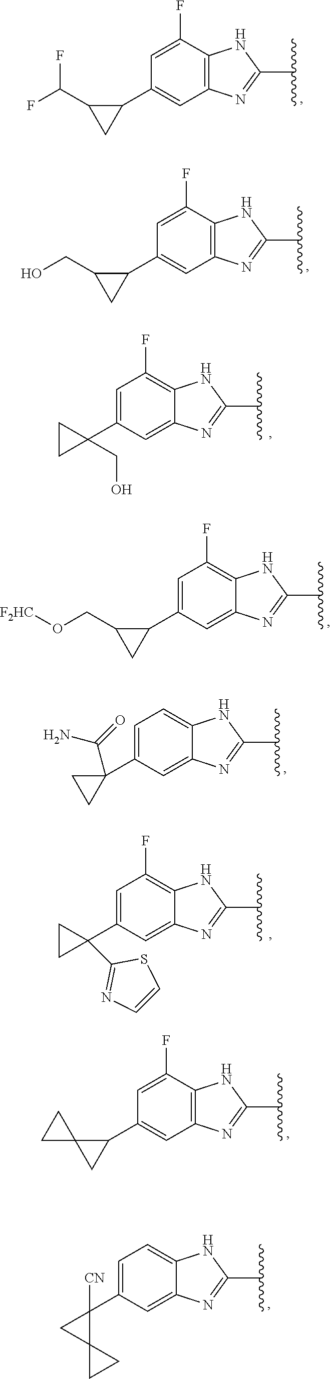

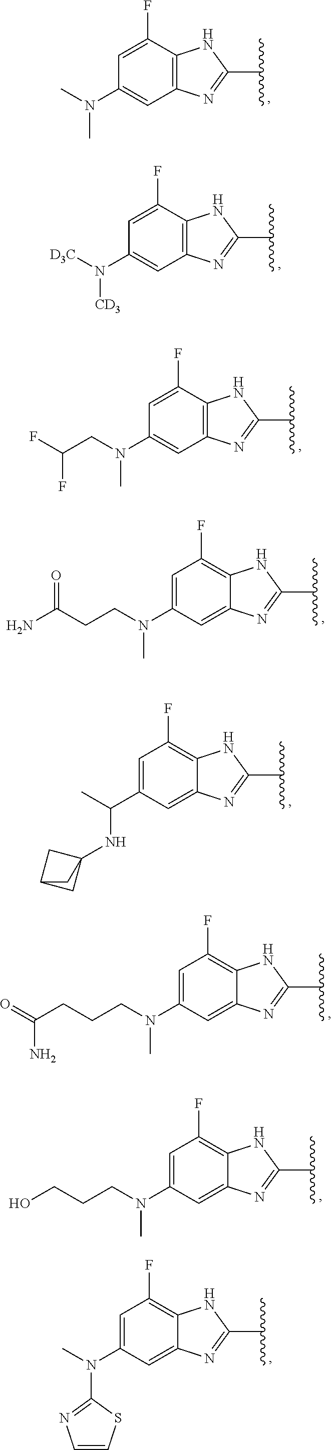

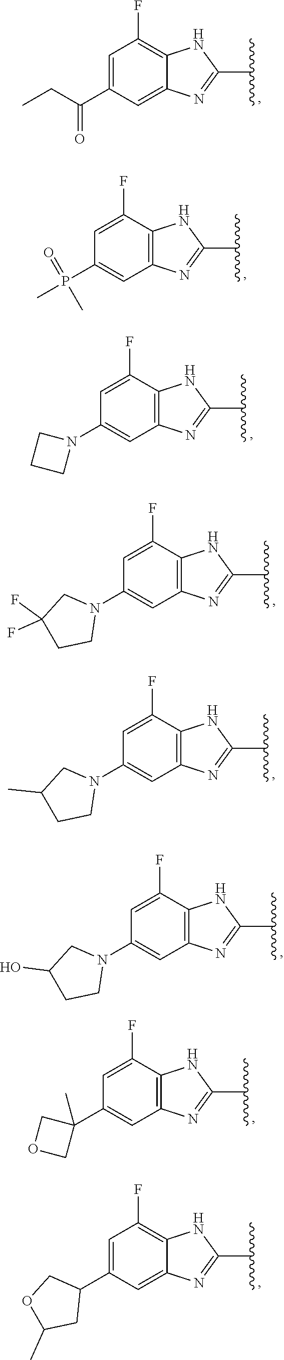

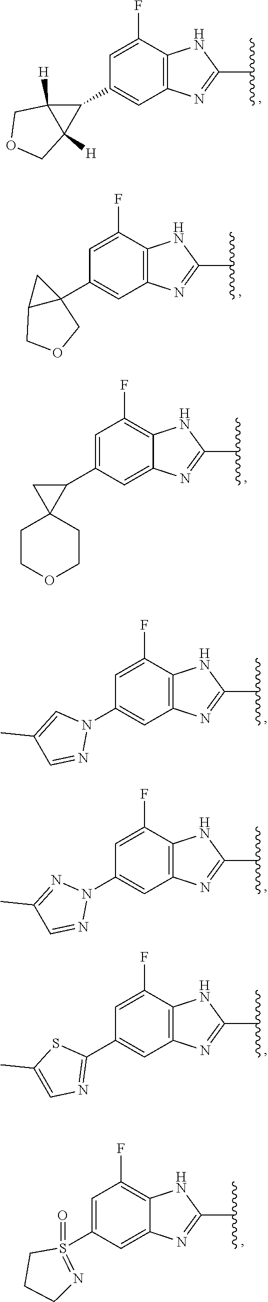

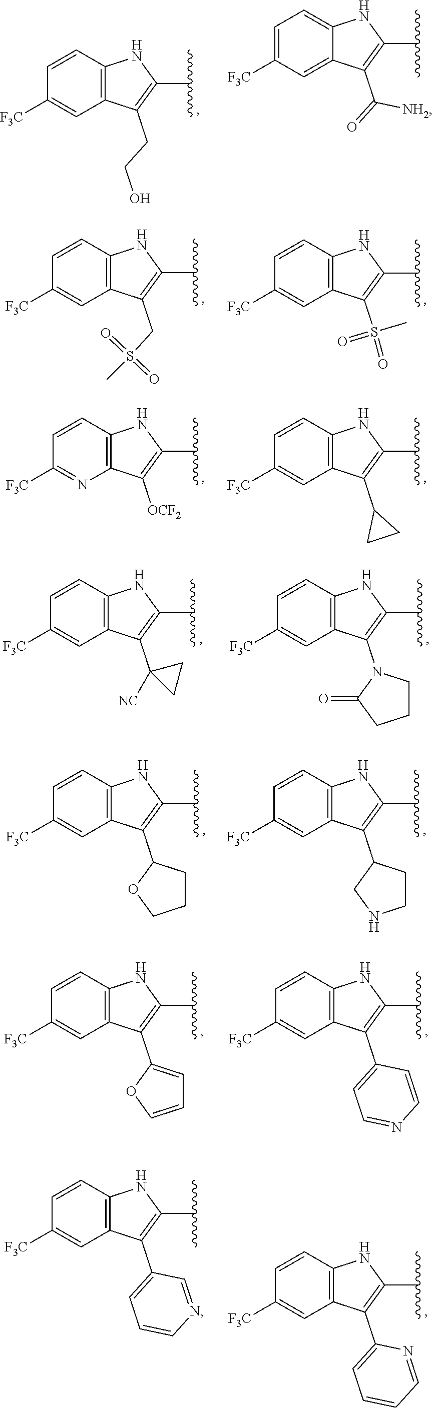

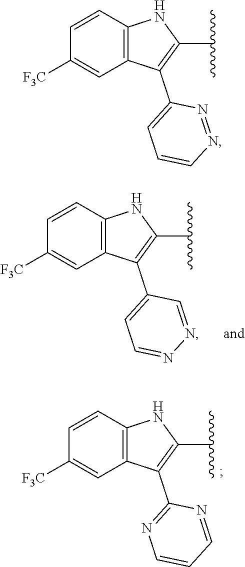 [SMILES: *c1[nH]c2ccc(C(F)(F)F)cc2c1-c1cccnn1 ; *c1[nH]c2ccc(C(F)(F)F)cc2c1-c1ccnnc1 ; *c1[nH]c2ccc(C(F)(F)F)cc2c1-c1ncccn1]

and all other variables not specifically defined herein are as defined in any one of the suitable preceding embodiments.

In a 20^{th} embodiment, in a compound, tautomer, a solvate or stereoisomer of the compound or the tautomer, or pharmaceutically acceptable salt of this disclosure, Ring B is selected from:

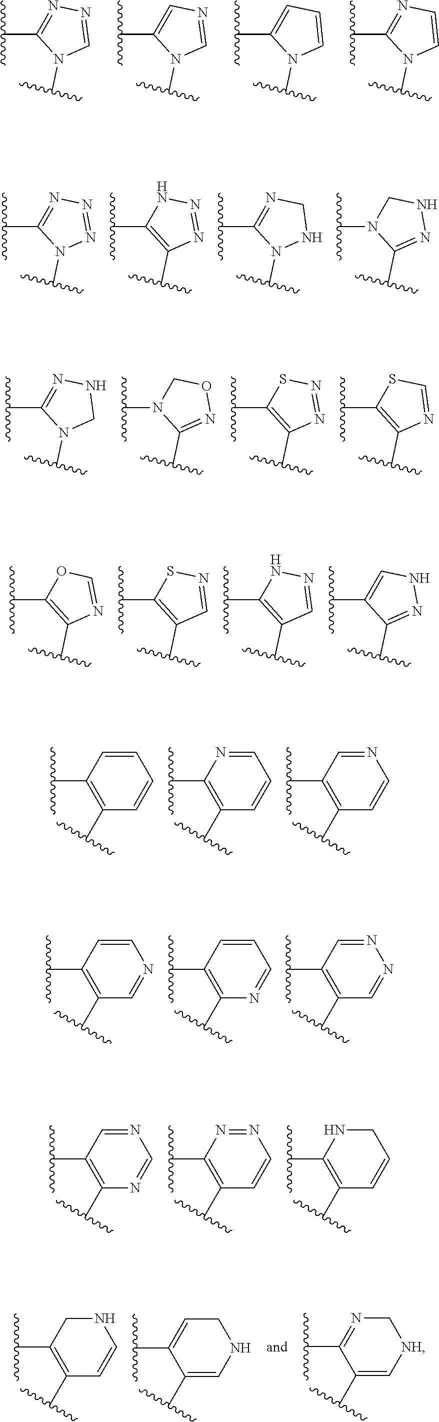

wherein Ring B is substituted with m groups of R⁶; and all other variables not specifically defined herein are as defined in any one of the suitable preceding embodiments.

In a 21^{st} embodiment, in a compound, tautomer, a solvate or stereoisomer of the compound or the tautomer, or pharmaceutically acceptable salt of this disclosure, Ring B is selected from:

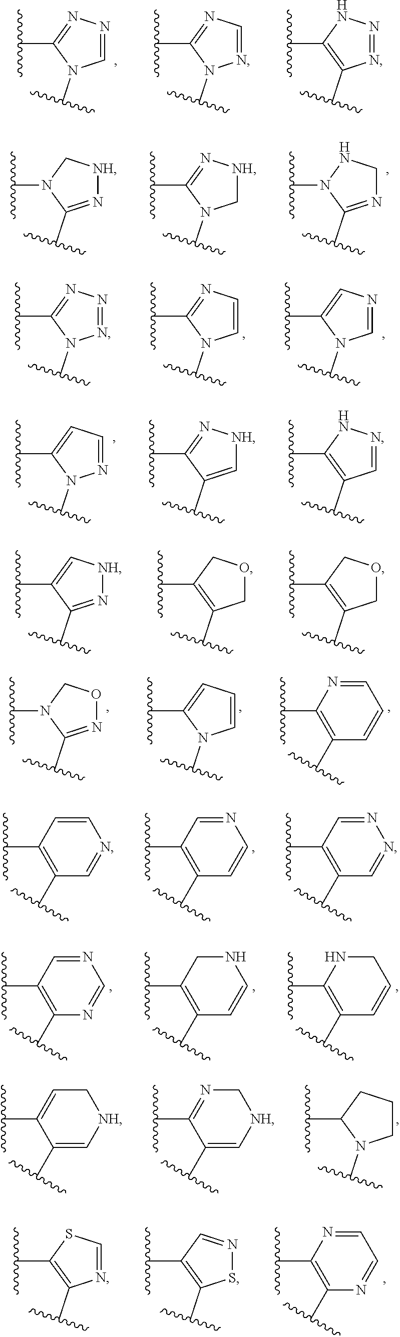

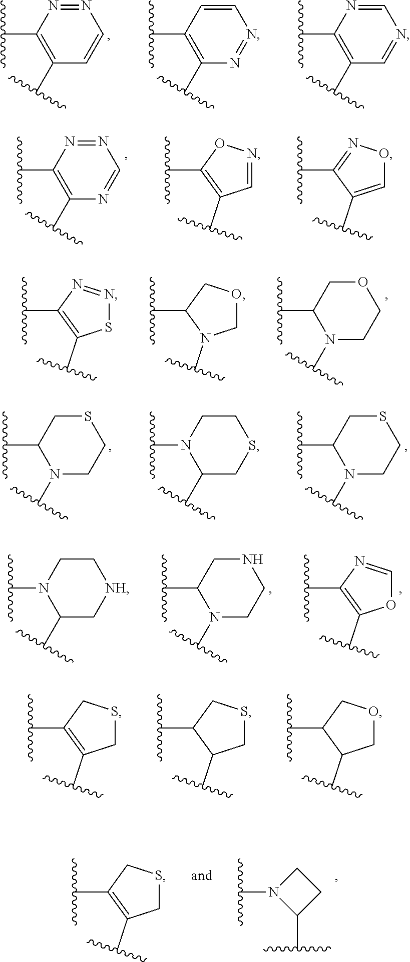

wherein Ring B is substituted with m groups of R⁶, and all other variables not specifically defined herein are as defined in any one of the suitable preceding embodiments.

In a 22^{nd} embodiment, in a compound, tautomer, a solvate or stereoisomer of the compound or the tautomer, or pharmaceutically acceptable salt of this disclosure,

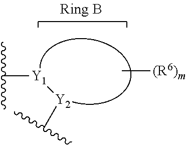

is selected from:

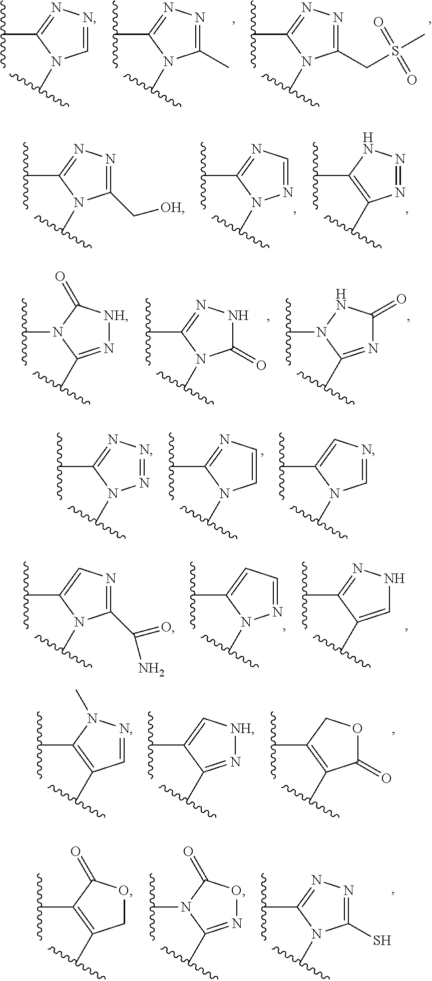

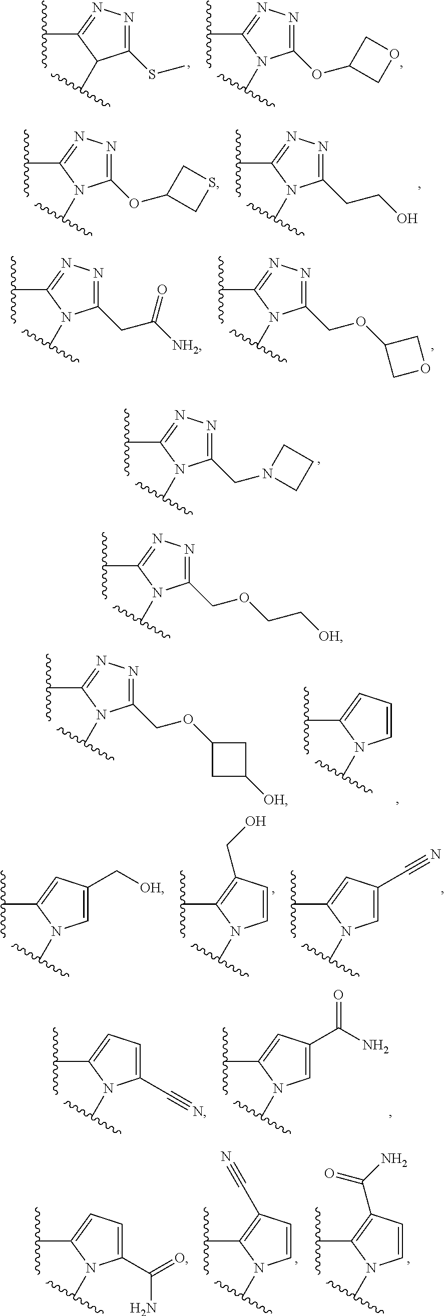

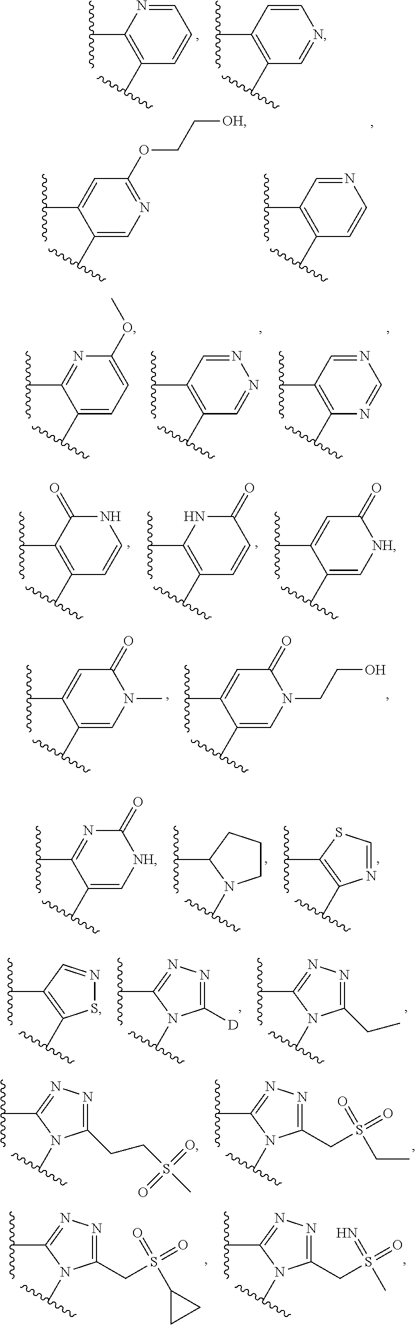

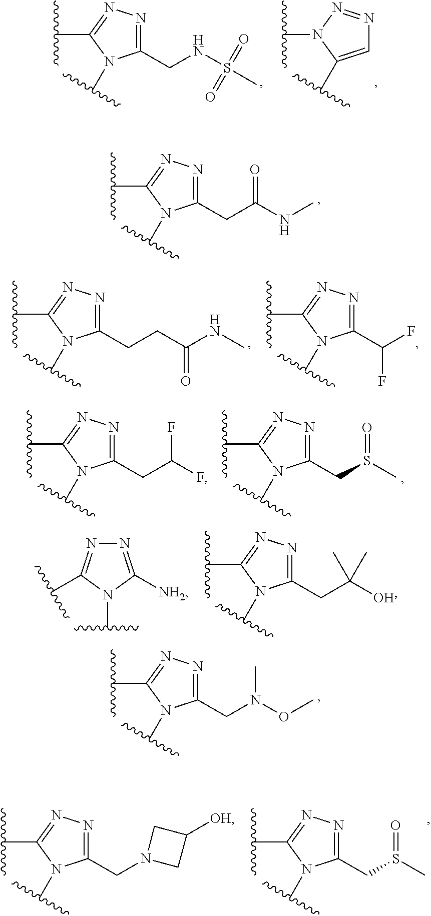

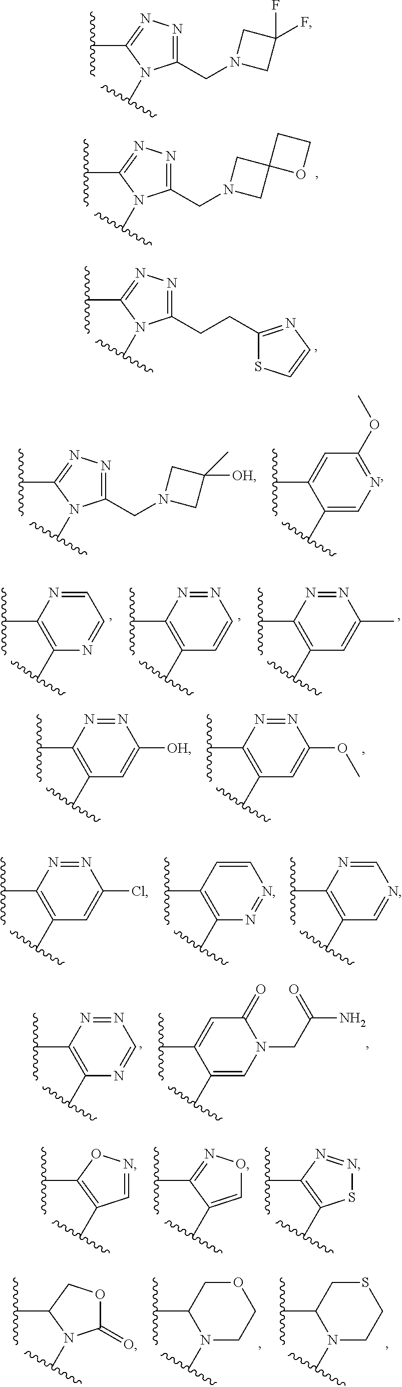

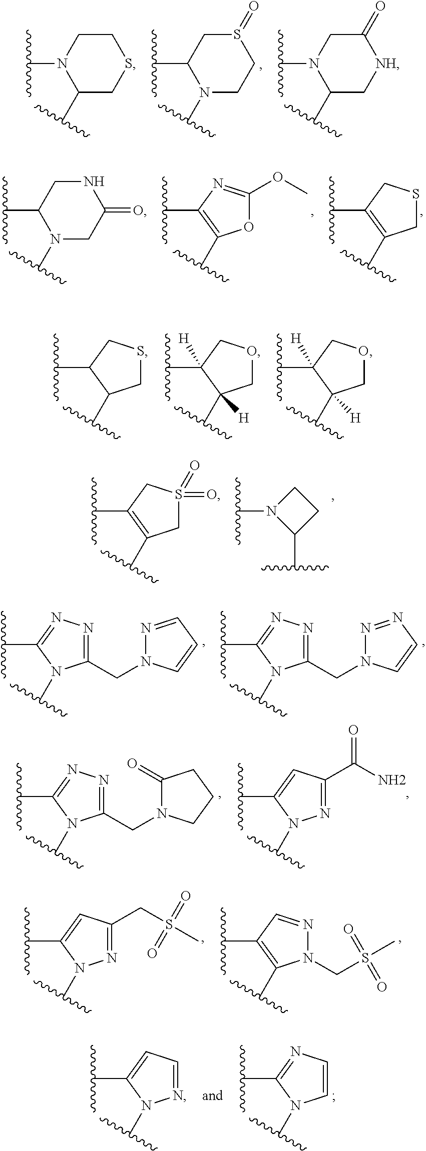

and all other variables not specifically defined herein are as defined in any one of the suitable preceding embodiments.

In a 23^{rd} embodiment, in a compound, tautomer, a solvate or stereoisomer of the compound or the tautomer, or pharmaceutically acceptable salt of this disclosure, Ring C is selected from:

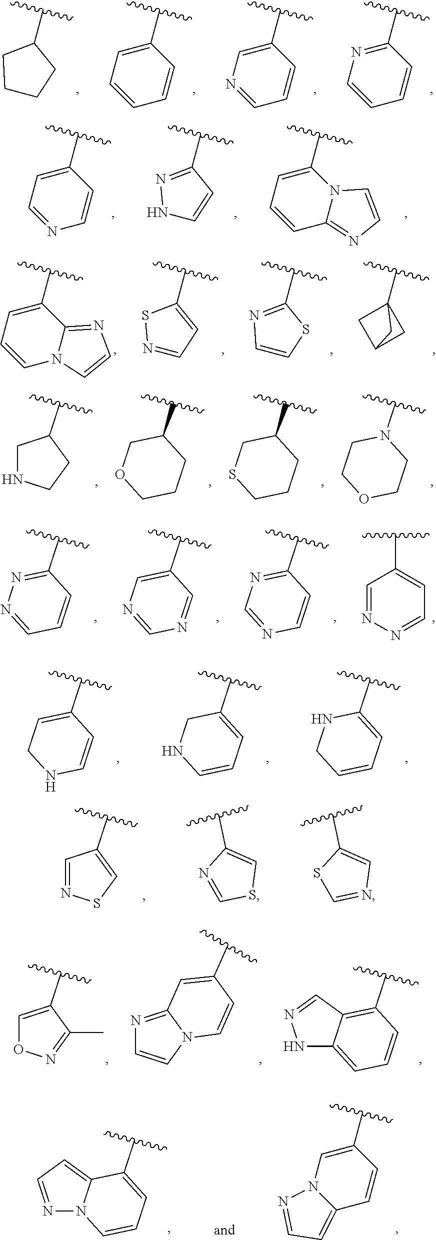

wherein Ring C is substituted with n groups of R⁷; and all other variables not specifically defined herein are as defined in any one of the suitable preceding embodiments.

In a 24^{th} embodiment, in a compound, tautomer, a solvate or stereoisomer of the compound or the tautomer, or pharmaceutically acceptable salt of this disclosure,

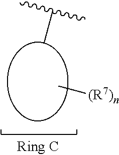

is selected from:

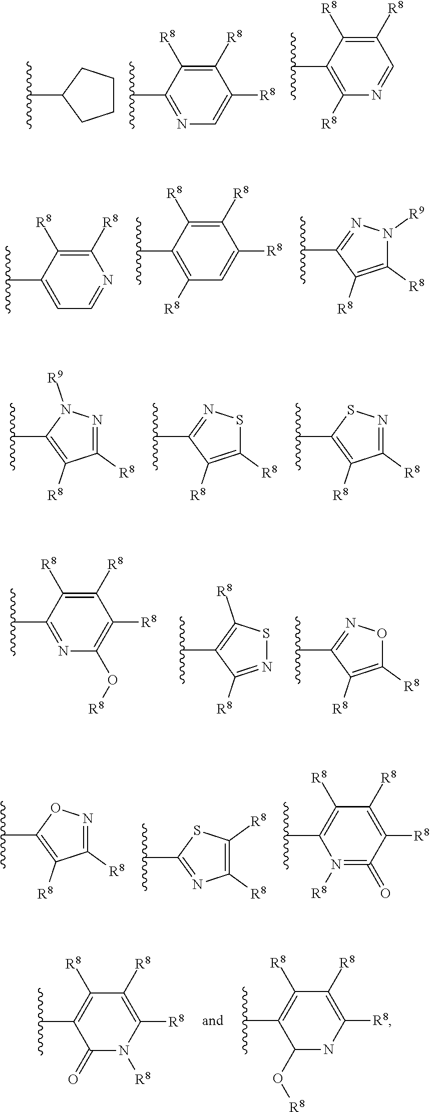

wherein:
R⁸, for each occurrence, is independently selected from H, F, Cl, Me, CHF₂, CF₃, CN, SO₂Me, SMe, CH₂CF₃, CH₂SO₂Me,

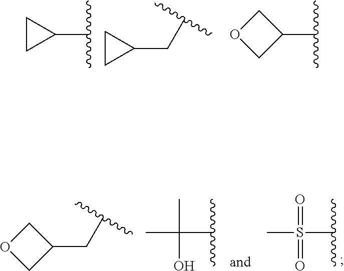

and
R⁹, for each occurrence, is independently selected from Me, CF₃, CHF₂, CH₂CF₃, Acetyl, SO₂Me,

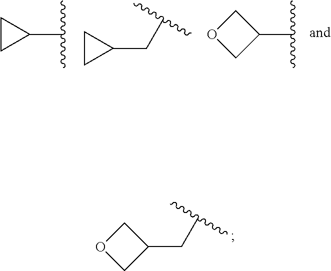

and all other variables not specifically defined herein are as defined in any one of the suitable preceding embodiments.

In a 25^{th} embodiment, in a compound, tautomer, a solvate or stereoisomer of the compound or the tautomer, or pharmaceutically acceptable salt of this disclosure,

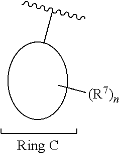

is selected from:

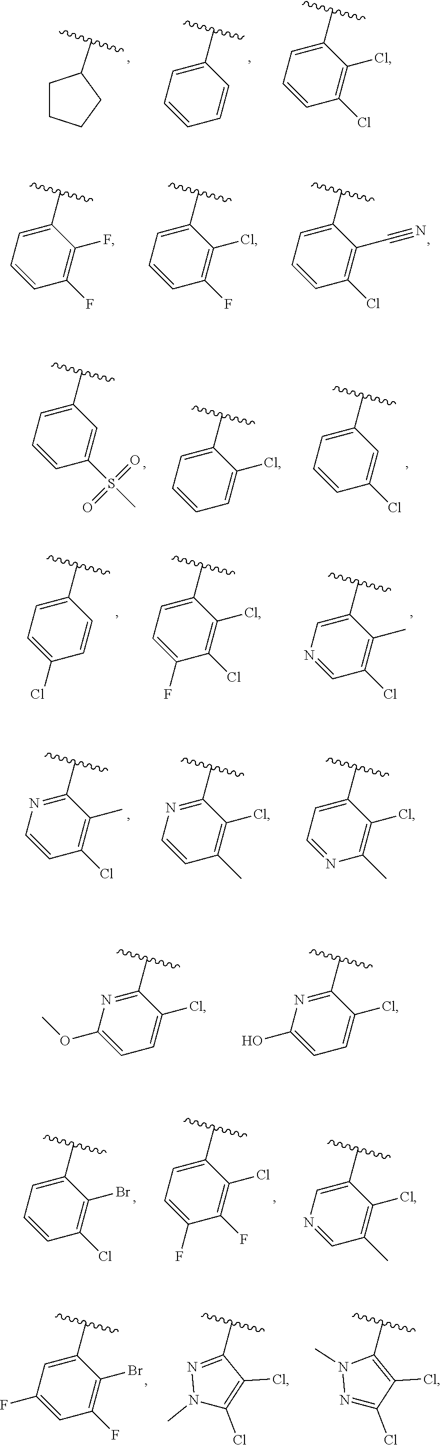

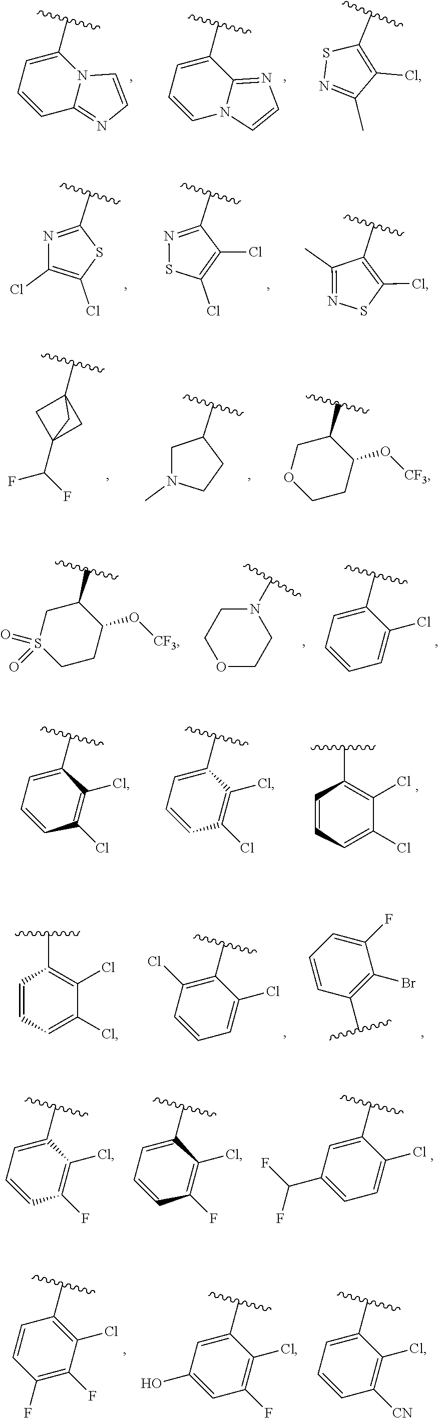

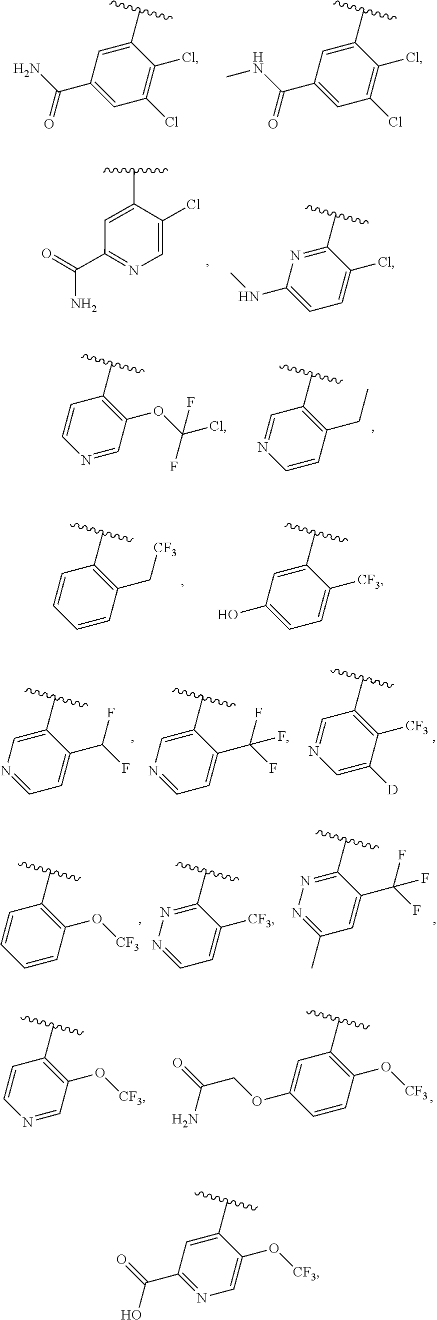

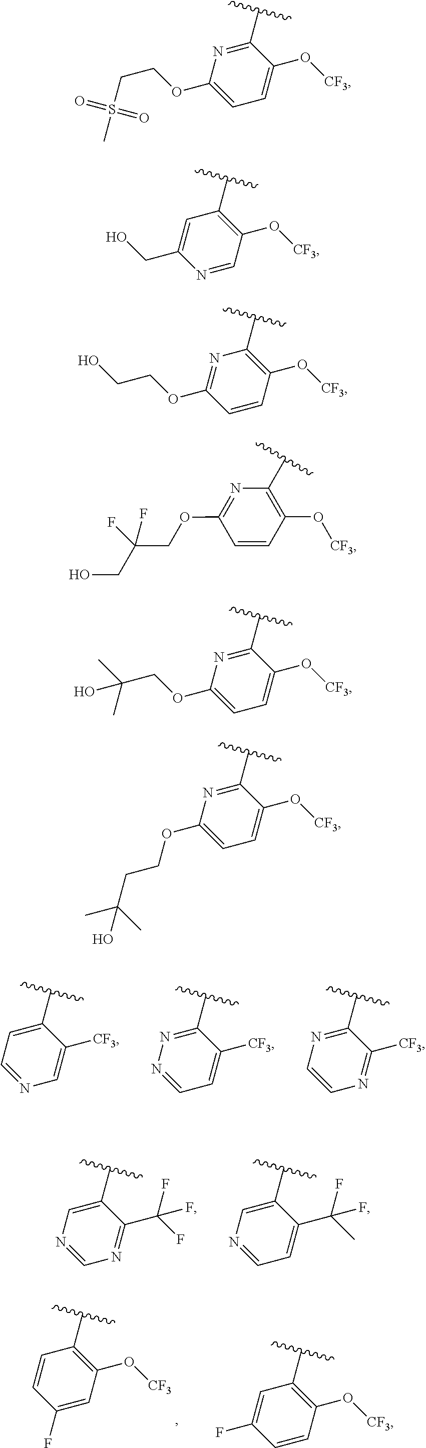

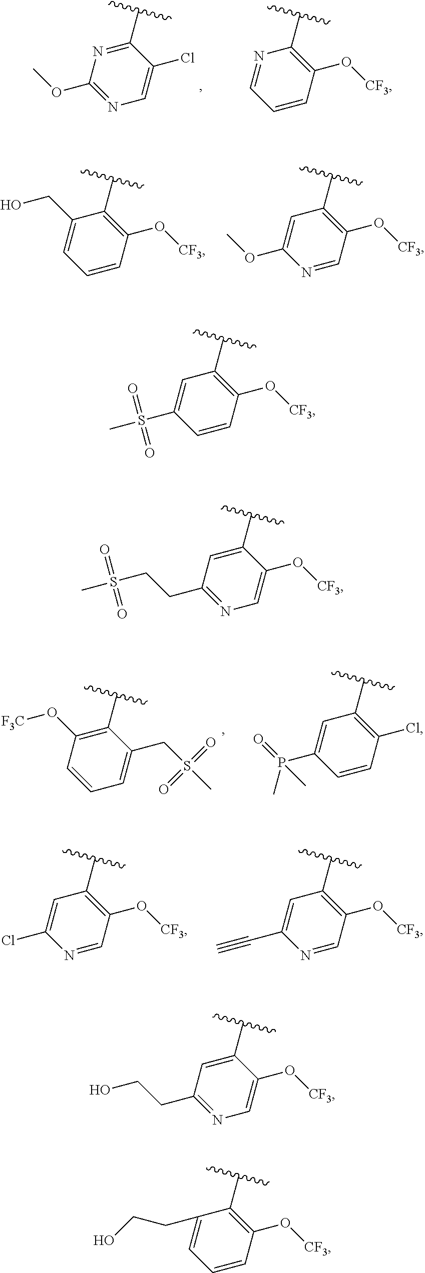

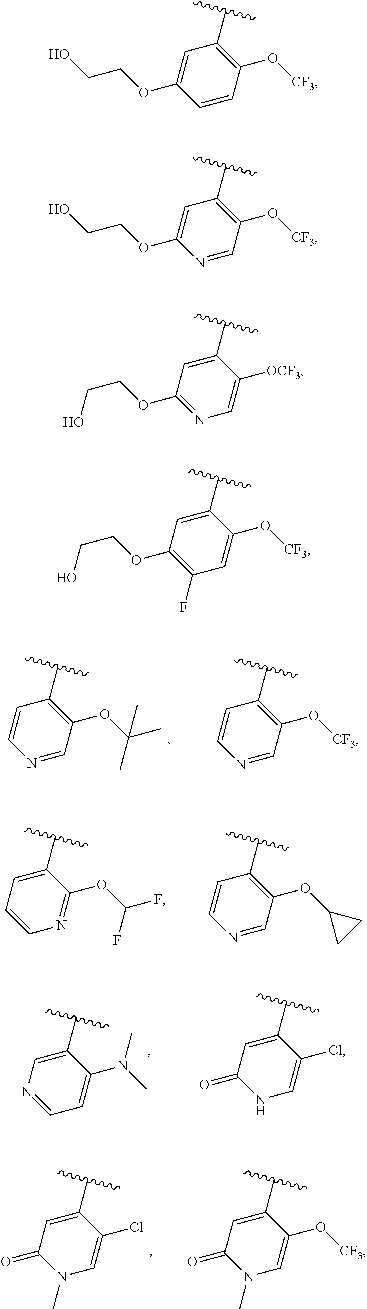

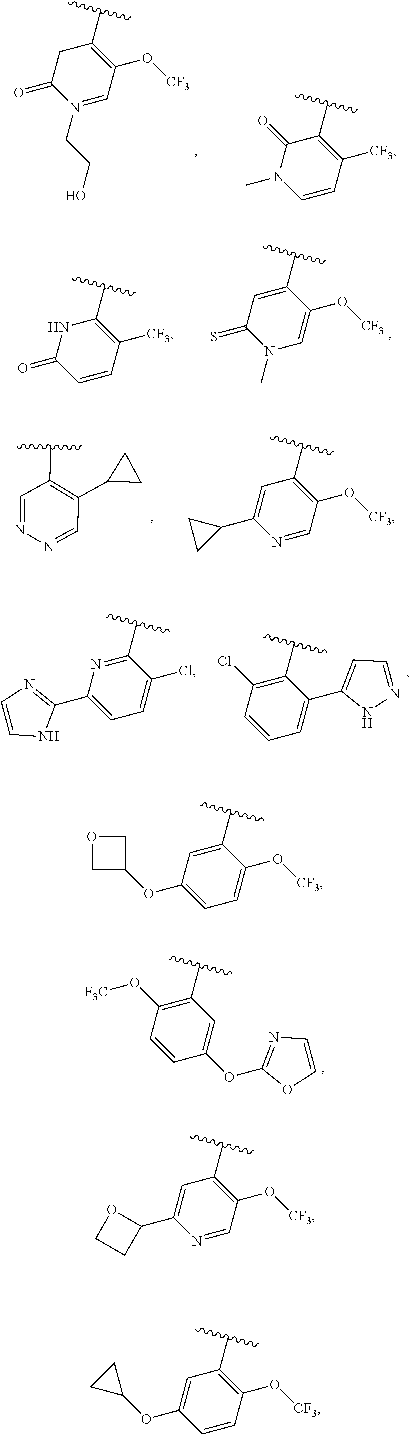

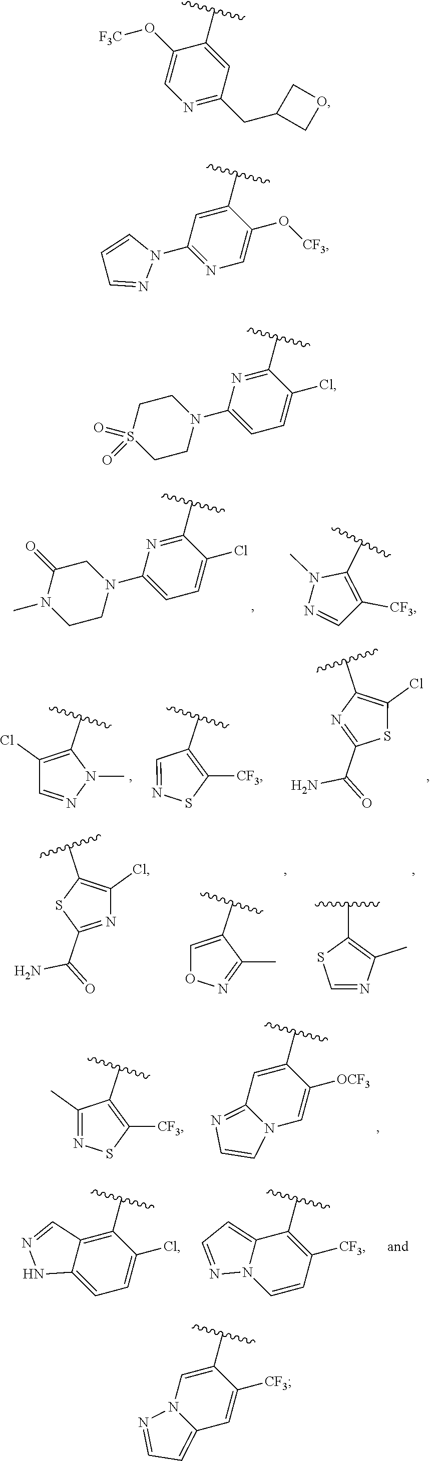

and all other variables not specifically defined herein are as defined in any one of the suitable preceding embodiments.

In a 26^{th} embodiment, in a compound, tautomer, a solvate or stereoisomer of the compound or the tautomer, or pharmaceutically acceptable salt of this disclosure, R¹ is selected from: H, Me, Cl, F, Br, OMe, CF₃, OCF₃, CHF₂, SO₂Me, CN, OH, CH₂OH, COOH, CONH₂,

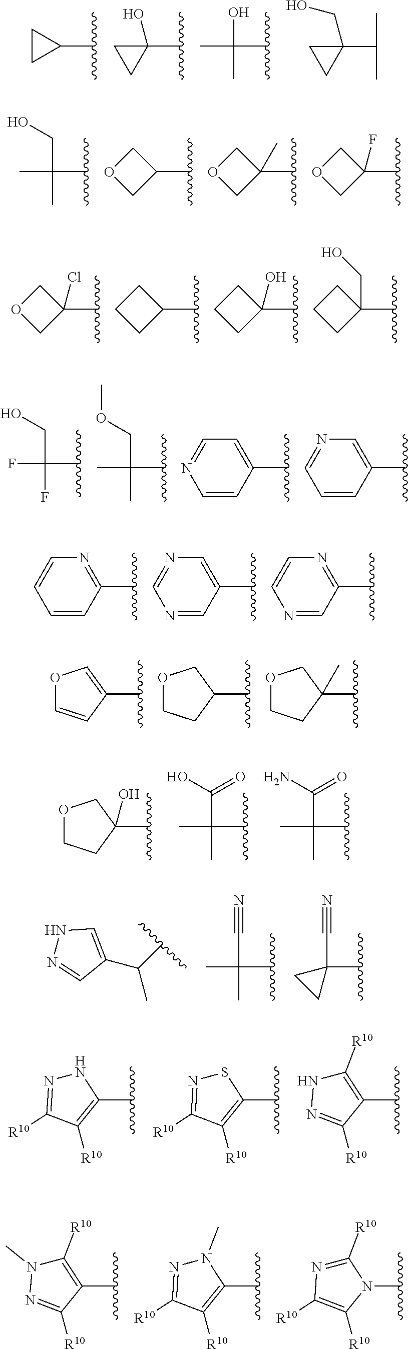

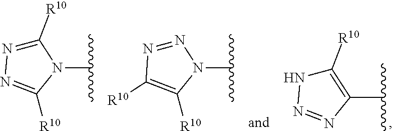

wherein R¹⁰, for each occurrence, is independently selected from H, Me, Cl, F, CF₃, and CN; and all other variables not specifically defined herein are as defined in any one of the suitable preceding embodiments.

In a 27^{th} embodiment, in a compound, tautomer, a solvate or stereoisomer of the compound or the tautomer, or pharmaceutically acceptable salt of this disclosure, R¹ is selected from: H, —CH₃, —CF₃, —CHF₂, —OCF₃, —C(CH₃)₂OH, Br, Cl, —S(═O)₂CH₃, —SF₅,

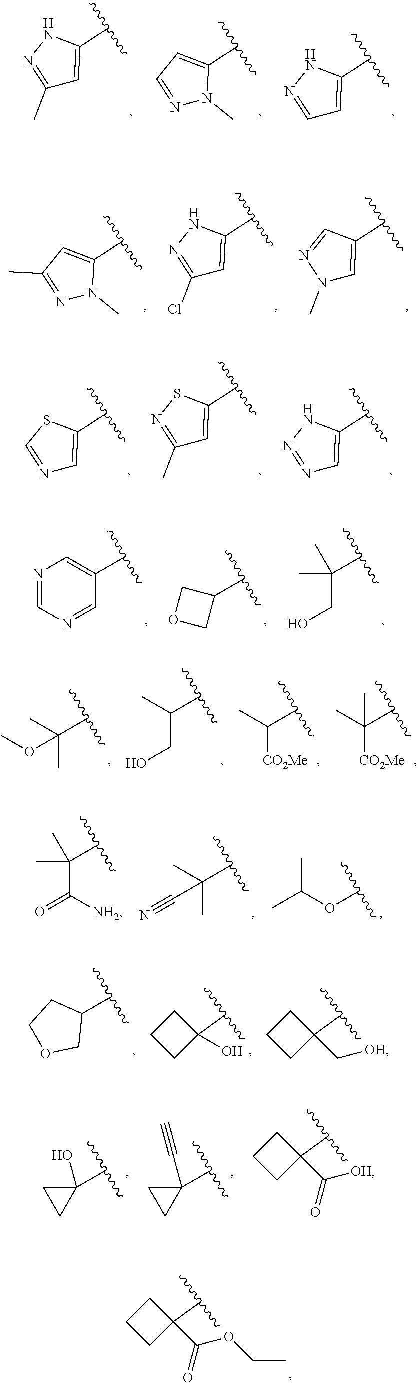

CH₂CH₃, C(CH₃)₃, —CH═CH₂, CH₂CHF₂, CH₂CF₃, CH₂OH, CH₂CN,

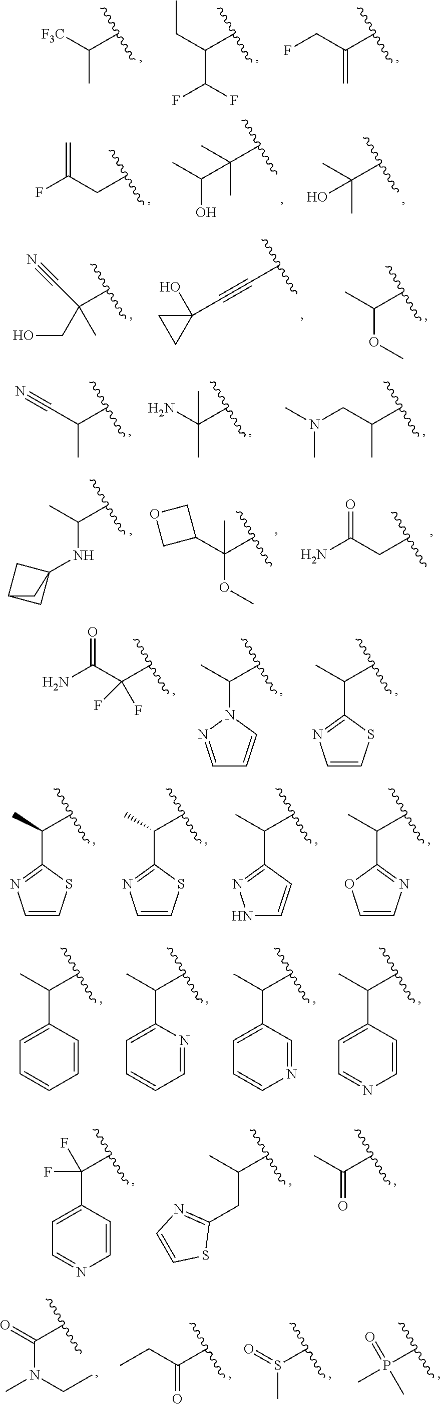

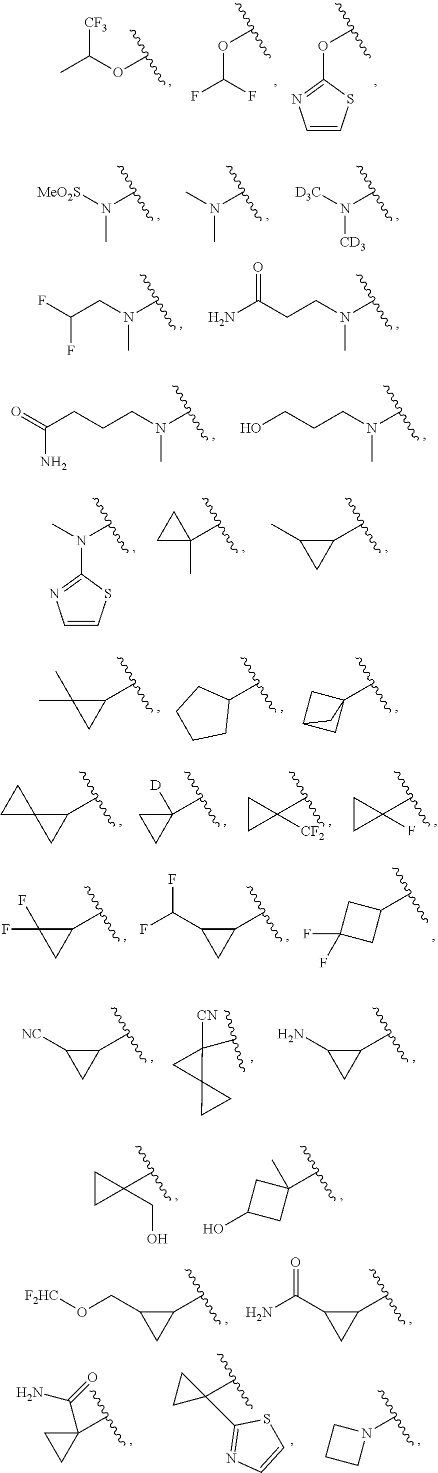

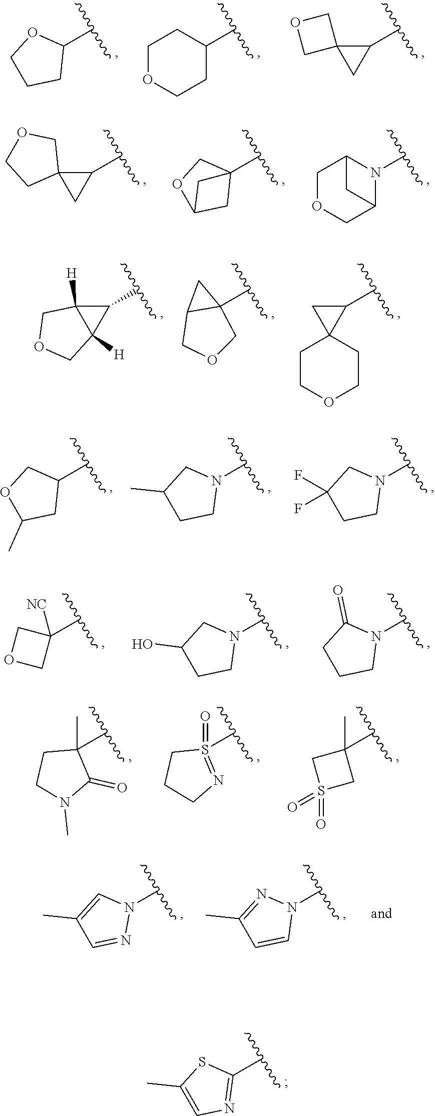

and all other variables not specifically defined herein are as defined in any one of the suitable preceding embodiments.

In a 28^{th} embodiment, in a compound, tautomer, a solvate or stereoisomer of the compound or the tautomer, or pharmaceutically acceptable salt of this disclosure, R¹ is selected from: halogen, —C(═O)R^{f}, —OR^{f}, —NR^{f}R^{g}, —SF₅,
C₁-C₆ alkyl (optionally substituted with 1 to 3 groups selected from F, —CN, —OR^{f}, phenyl, —NR^{f}R^{g}, and 5- to 6-membered heteroaryl),
C₁-C₆ alkenyl (optionally substituted with 1 to 3 groups selected from F, —CN, —OR^{f}, phenyl, —NR^{f}R^{g}, and 5- to 6-membered heteroaryl),
3- to 6-membered cycloalkyl (optionally substituted with 1-2 groups selected from D, halogen-, —CN, R^{f}, —OR^{f}, CH₂OR^{f}, —C(═O)NR^{f}R^{g}, and 5- to 6-membered heteroaryl),
4- to 8-membered heterocyclyl (optionally substituted with 1-2 groups selected from R^{f}, —OR^{f}, halogen, and —CN), and
5- to 6-membered heteroaryl (optionally substituted with 1-2 groups selected from R^{f}, —OR^{f}, halogen, and —CN),
wherein:
R^{f} and R^{g}, for each occurrence, are each independently selected from H, 5- to 6-membered heteroaryl, 3- to 6-membered cycloalkyl, and C₁-C₃ alkyl (optionally substituted with 1 to 3 groups selected from D, halogen, —OH, —OCH₃, —C(═O)NH₂, and —CN); and all other variables not specifically defined herein are as defined in any one of the suitable preceding embodiments.

In a 29^{th} embodiment, in a compound, tautomer, a solvate or stereoisomer of the compound or the tautomer, or pharmaceutically acceptable salt of this disclosure, R¹ is selected from: CF₃, F, Cl, C₁-C₃ alkyl and C₃-C₅ cycloalkyl; and all other variables not specifically defined herein are as defined in any one of the suitable preceding embodiments.

In a 30^{th} embodiment, in a compound, tautomer, a solvate or stereoisomer of the compound or the tautomer, or pharmaceutically acceptable salt of this disclosure, R² is selected from: H, Me, Cl, F, Br, —OMe, CF₃, —CN, —CONH₂, —SO₂Me, —S(═O)CH₃, —SCH₃,

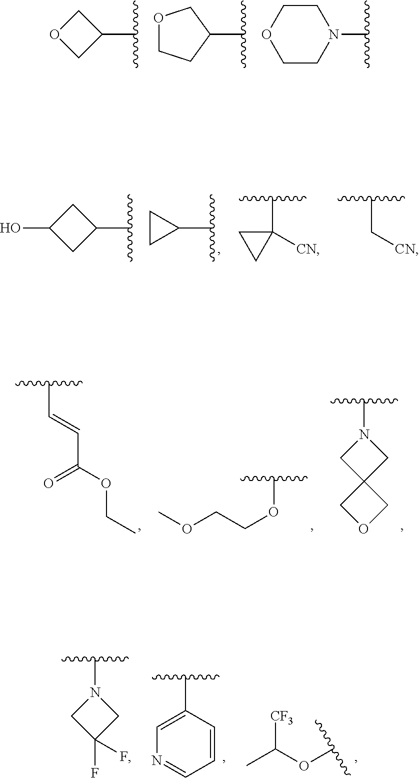

and —OH; and all other variables not specifically defined herein are as defined in any one of the suitable preceding embodiments.

In a 31^{st} embodiment, in a compound, tautomer, a solvate or stereoisomer of the compound or the tautomer, or pharmaceutically acceptable salt of this disclosure, R² is selected from: —CH₃, —S(═O)CH₃, —SCH₃, —CN, and S(═O)₂CH₃; and all other variables not specifically defined herein are as defined in any one of the suitable preceding embodiments.

In a 32^{nd} embodiment, in a compound, tautomer, a solvate or stereoisomer of the compound or the tautomer, or pharmaceutically acceptable salt of this disclosure, R³ and R⁴ are each independently selected from H, Me, Cl, F, Br, and OMe; and all other variables not specifically defined herein are as defined in any one of the suitable preceding embodiments.

In a 33^{rd} embodiment, in a compound, tautomer, a solvate or stereoisomer of the compound or the tautomer, or pharmaceutically acceptable salt of this disclosure, R³ and R⁴ are each independently selected from H, Me, Cl, F, Br, OMe, CF₃, and

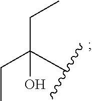 [SMILES: *C(O)(CC)CC]

and all other variables not specifically defined herein are as defined in any one of the suitable preceding embodiments.

In a 34^{th} embodiment, in a compound, tautomer, a solvate or stereoisomer of the compound or the tautomer, or pharmaceutically acceptable salt of this disclosure, R⁴ is selected from F and C₁; and all other variables not specifically defined herein are as defined in any one of the suitable preceding embodiments.

In a 35^{th} embodiment, in a compound, tautomer, a solvate or stereoisomer of the compound or the tautomer, or pharmaceutically acceptable salt of this disclosure, R⁵ is selected from —CN and —CH₂OH; and all other variables not specifically defined herein are as defined in any one of the suitable preceding embodiments.

In a 36^{th} embodiment, in a compound, tautomer, a solvate or stereoisomer of the compound or the tautomer, or pharmaceutically acceptable salt of this disclosure, wherein R⁵ is selected from: absent, H, —CN, —CH₂OH,

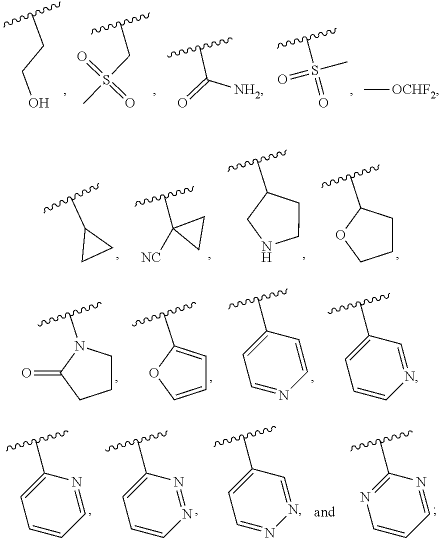

and all other variables not specifically defined herein are as defined in any one of the suitable preceding embodiments.

In a 37^{th} embodiment, in a compound, tautomer, a solvate or stereoisomer of the compound or the tautomer, or pharmaceutically acceptable salt of this disclosure, R⁵ is selected from: absent, H, CN, halogen, —S(═O)₂CH₃, —CH₂S(═O)₂CH₃, 3- to 4-membered cycloalkyl, 5- to 6-membered heterocyclyl, 5- to 6-membered heteroaryl, CH₂OH, and CH₂CH₂OH; and all other variables not specifically defined herein are as defined in any one of the suitable preceding embodiments.

In a 38^{th} embodiment, in a compound, tautomer, a solvate or stereoisomer of the compound or the tautomer, or pharmaceutically acceptable salt of this disclosure, R⁶ is selected from:

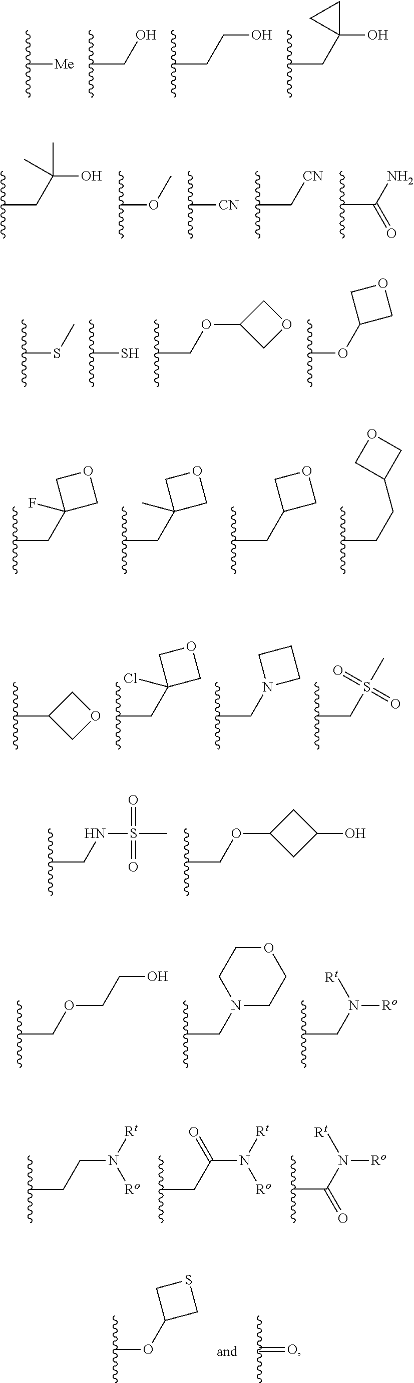

wherein R^{t} and R^{o} are each independently selected from H and C₁-C₆ alkyl, wherein the C₁-C₆ alkyl of R^{t} and R^{o} is optionally substituted with 1-3 groups selected from halogen; and all other variables not specifically defined herein are as defined in any one of the suitable preceding embodiments.

In a 39^{th} embodiment, in a compound, tautomer, a solvate or stereoisomer of the compound or the tautomer, or pharmaceutically acceptable salt of this disclosure, R⁶ is selected from: —CN, ═O, —CH₃, —CH₂S(═O)₂CH₃, —CH₂OH, —CH₂CH₂OH, —C(═O)NH₂, —O(CH₂)₂OH, —OCH₃, —SH, —SCH₃,

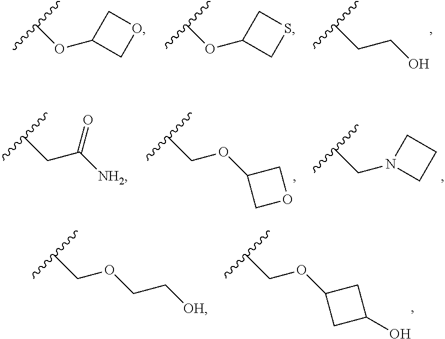

D, Cl, CH₂CH₃, CHF₂, CH₂CHF₂,

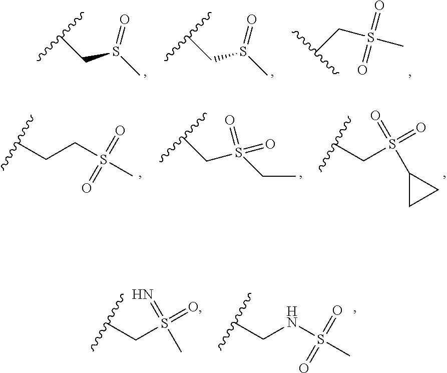

CH₂CONH₂,

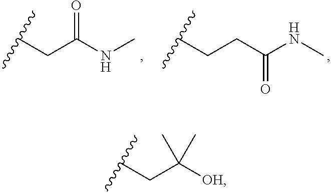

### —NH₂,

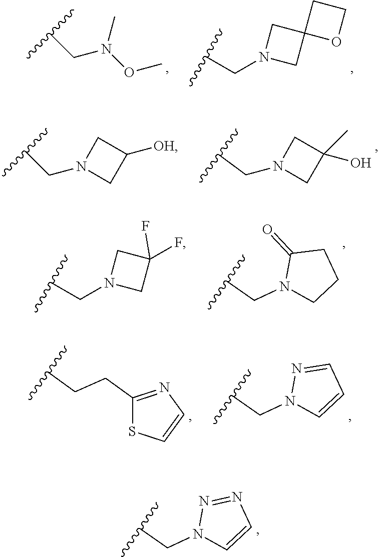

OH, —OCH₃, and ═O; and all other variables not specifically defined herein are as defined in any one of the suitable preceding embodiments.

In a 40^{th} embodiment, in a compound, tautomer, a solvate or stereoisomer of the compound or the tautomer, or pharmaceutically acceptable salt of this disclosure, R⁶ is selected from:

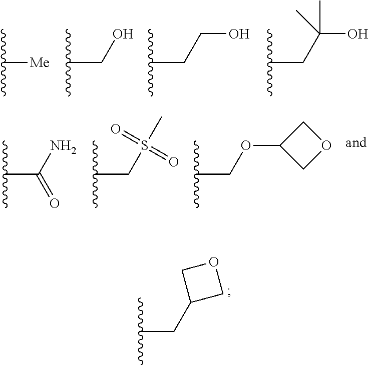

and all other variables not specifically defined herein are as defined in any one of the suitable preceding embodiments.

In a 41^{st} embodiment, in a compound, tautomer, a solvate or stereoisomer of the compound or the tautomer, or pharmaceutically acceptable salt of this disclosure, R⁶ is selected from D, halogen, —S(═O)₂R^{h}, —NR^{h}Rⁱ, —C(═O)NR^{h}Rⁱ, and
C₁-C₄ alkyl (optionally substituted with 1 to 3 groups selected from halogen, —OR^{h}, —C(═O)NR^{h}Rⁱ, —NR^{h}Rⁱ, —S(═O)₂R^{h}, —NHS(═O)ₚR^{h}, —S(═O)R^{h},

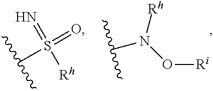

5- to 6-membered heteroaryl, 3- to 5-membered cycloalkyl (optionally substituted with 1 to 3 groups selected from halogen and R^{h}), and 3- to 8-membered heterocyclyl optionally substituted with 1 to 3 groups selected from halogen and R^{h}),
wherein:
R^{h} and Rⁱ, for each occurrence, are each independently selected from H, C₁-C₃ alkyl (optionally substituted with 1 to 3 groups selected from halogen, —OH, —O(C₁-C₃ alkyl), and —S(═O)₂CH₃), and 3- to 5-membered cycloalkyl; and all other variables not specifically defined herein are as defined in any one of the suitable preceding embodiments.

In a 42^{nd} embodiment, in a compound, tautomer, a solvate or stereoisomer of the compound or the tautomer, or pharmaceutically acceptable salt of this disclosure, wherein R⁷ is selected from F, Cl, Me, CHF₂, CF₃, CN, —SO₂Me, —SMe, CH₂CF₃, CH₂SO₂Me, acetyl,

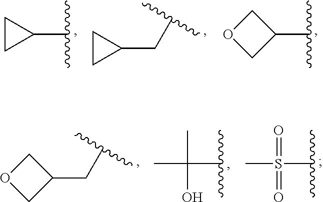

D, Br, CN, CH₂CH₃, CH₂CF₃, CF₂CH₃, CH₂OH, CH₂CH₂OH,

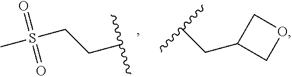

—OH, —OC(CH₃)₃, —OCF₃, —OCHF₂, —OCH₂CH₂OH,

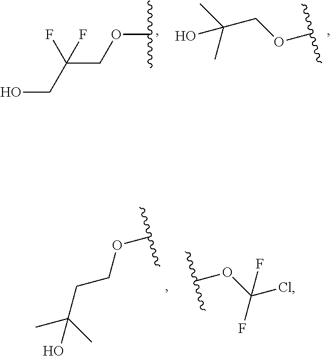

OCH₂CONH₂,

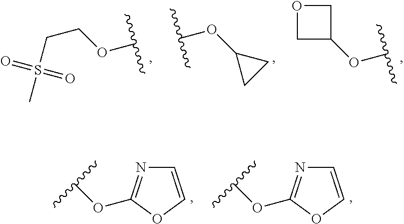

—COOH, —CONH₂, —CONHCH₃, ═O, ═S, —SO₂CH₃,

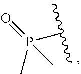 [SMILES: *P(C)(C)=O]

—NHCH₃, —N(CH₃)₂,

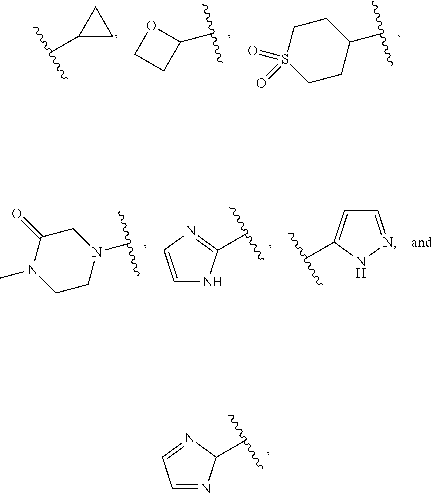

and n is 0, 1, 2, or 3; and all other variables not specifically defined herein are as defined in any one of the suitable preceding embodiments.

In a 43^{rd} embodiment, in a compound, tautomer, a solvate or stereoisomer of the compound or the tautomer, or pharmaceutically acceptable salt of this disclosure, R⁷ is selected from: Cl, F, —CN, —S(═O)₂CH₃, —CH₃, —OCH₃, and —OH; and all other variables not specifically defined herein are as defined in any one of the suitable preceding embodiments.

In a 44^{th} embodiment, in a compound, tautomer, a solvate or stereoisomer of the compound or the tautomer, or pharmaceutically acceptable salt of this disclosure, R⁷ is selected from: D, halogen, CN, ═O, ═S, —OR^{j}, —NR^{j}R^{k}, —C(═O)NR^{j}R^{k}, —C(═O)OR^{j}, —S(═O)₂CH₃, 3- to 5-membered cycloalkyl, 5- to 6-membered heteroaryl,
C₁-C₆ alkyl (optionally substituted with 1 to 3 groups selected from halogen, —OH, —S(═O)₂CH₃, and 4- to 6-membered heterocyclyl), and
4- to 6-membered heterocyclyl (optionally substituted with 1 to 2 groups selected from ═O and R^{k}),
wherein:
R^{j} and R^{k}, for each occurrence, are independently selected from H, C₁-C₆ alkyl (optionally substituted with 1 to 3 groups selected from halogen, OH, —C(═O)NH₂, and —S(═O)₂CH₃), 4- to 6-membered heterocyclyl, and 3- to 5-membered cycloalkyl; and all other variables not specifically defined herein are as defined in any one of the suitable preceding embodiments.

In a 45^{th} embodiment, in a compound, tautomer, a solvate or stereoisomer of the compound or the tautomer, or pharmaceutically acceptable salt of this disclosure,
R¹ is selected from CH₃, CF₃, OCF₃, S(═O)₂CH₃, Br, Cl,

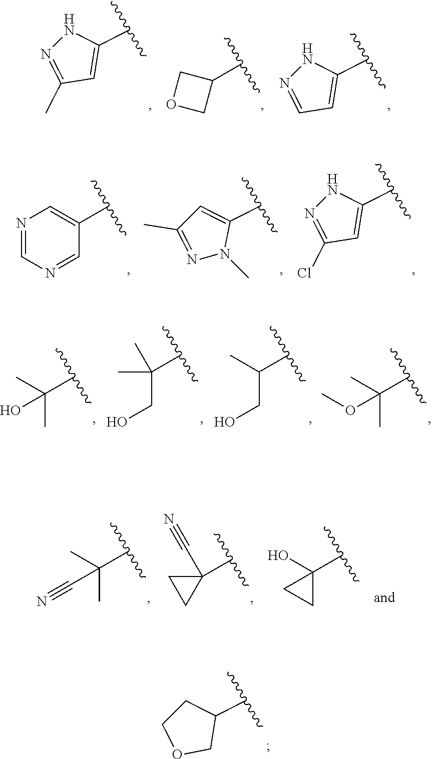

R² is selected from H, CN, and S(═O)₂CH₃,
R³, R⁴, and R⁵ are H,
R⁶ is selected from ═O, CH₃, Cl, —C(═O)NH₂, —CH₂CH₂OH, —CH₂OH, and —CH₂S(═O)₂CH₃, m is 0, 1, or 2,
R⁷ is selected from CH₃, Cl, F, CN, OCH₃, and OH, and n is 0, 1, 2, or 3; and all other variables not specifically defined herein are as defined in any one of the suitable preceding embodiments.

In a 46^{th} embodiment, in a compound, tautomer, a solvate or stereoisomer of the compound or the tautomer, or pharmaceutically acceptable salt of this disclosure,
R¹ is selected from C₁-C₆ alkyl (optionally substituted with 1 to 3 groups selected from halogen, —OH, CN, —OCH₃, —NR^{d}R^{e}, phenyl, 5-membered heteroaryl containing 1 to 3 heteroatoms selected from N, O, and S, and 6-membered heteroaryl containing 1 to 3 nitrogen atoms), C₂-C₄ alkenyl (optionally substituted with 1 to 3 groups selected from halogen, —OH, —CN, and —OCH₃), —OR^{d}, —NR^{d}R^{e}, —S(═O)ₚCH₃, —SF₅, halogen, —C(═O)CH₃,

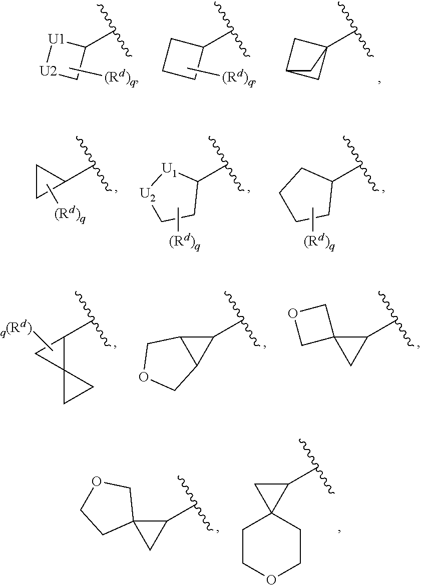

5-membered heteroaryl containing 1 to 2 heteroatoms selected from N and S (optionally substituted with 1 to 2 groups selected from C₁-C₃ alkyl), and 6-membered heteroaryl containing 1 to 2 nitrogen atoms (optionally substituted with 1 to 2 groups selected from C₁-C₃ alkyl);
R² is selected from H, CN, CH₃, F, and S(═O)₂CH₃; R³ is H; R⁴ is selected from F, Cl, and H;
R⁵ is selected from absent, H, F, —CN, —C(═O)NH₂, 3- to 4-membered cycloalkyl (optionally substituted with 1 to groups selected from CN and halogen), C₁-C₄ alkyl (optionally substituted with 1 to 3 groups selected from halogen, —S(═O)₂CH₃, and —OH), 5- to 6-membered heterocyclyl, —S(═O)₂CH₃, 5-membered heteroaryl containing 1 to 3 heteroatoms selected from N and 0, and 6-membered heteroaryl containing 1 to 3 nitrogen atoms;
R⁶ is selected from absent, D, C₁-C₄ alkyl (optionally substituted with 1-3 groups selected from halogen, —S(═O)CH₃, —S(═O)₂CH₃, —C(═O)NHCH₃, —OH, —C(═O)NH₂, —NR^{d}R^{e}, —NR^{d}OR^{e}, and —NHS(═O)₂CH₃), ═O, Cl, and —C(═O)NH₂;
R⁷ is selected from D, halogen, CF₃, —OCF₃, CN, —OR^{d}, —NR^{d}R^{e}, —C(═O)OH, ═O, ═S, —S(═O)₂CH₃, —C(═O)NR^{d}R^{e}, C₁-C₆ (optionally submitted with 1-3 groups selected from halogen, —OH, —S(═O)₂CH₃, —C(═O)₂NH₂, and 3- to 6-membered heterocyclyl), 5- to 6-membered heteroaryl, 3- to 5-membered cycloalkyl, and 4- to 6-membered heterocyclyl (optionally substituted with 1 to 3 groups selected from ═O and C₁-C₃ alkyl);
wherein:
R^{d} and R^{e}, for each occurrence, are each independently selected from H, C₁-C₄ alkyl (optionally substituted with 1 to 3 groups selected from D, halogen, —OH, CN, —C(═O)NH₂, —S(═O)₂CH₃, and —OCH₃), 5- to 6-membered heteroaryl, 4- to 6-membered heterocyclyl, and 3- to 5-membered cycloalkyl;
q is 0, 1, 2, or 3;
U₁ and U₂ are independently selected from O and C;
   and all other variables not specifically defined herein are as defined in any one of the suitable preceding embodiments.

In certain embodiments, a compound of the disclosure is selected from Compounds 1 to 468 depicted in Table 1, a tautomer thereof, a solvate or stereoisomer of the compound or the tautomer, or a pharmaceutically acceptable salt of the foregoing.

TABLE 1
Compounds 1 to 468

| | |
|---|---|
| 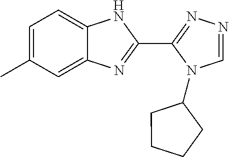 [SMILES: Cc1ccc2[nH]c(-c3nncn3C3CCCC3)nc2c1] | 1 |
| 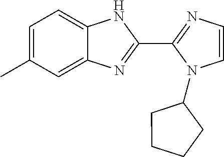 [SMILES: Cc1ccc2[nH]c(-c3nccn3C3CCCC3)nc2c1] | 2 |
| 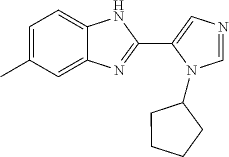 [SMILES: Cc1ccc2[nH]c(-c3cncn3C3CCCC3)nc2c1] | 3 |
| 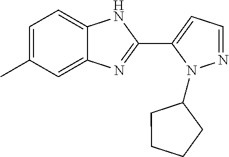 [SMILES: Cc1ccc2[nH]c(-c3ccnn3C3CCCC3)nc2c1] | 4 |
| 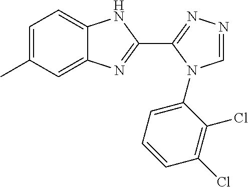 [SMILES: Cc1ccc2[nH]c(-c3nncn3-c3cccc(Cl)c3Cl)nc2c1] | 5 |
| 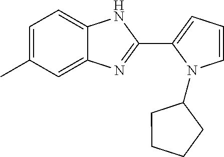 [SMILES: Cc1ccc2[nH]c(-c3cccn3C3CCCC3)nc2c1] | 6 |
| 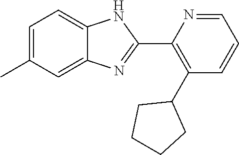 [SMILES: Cc1ccc2[nH]c(-c3ncccc3C3CCCC3)nc2c1] | 7 |
| 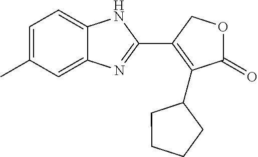 [SMILES: Cc1ccc2[nH]c(C3=C(C4CCCC4)C(=O)OC3)nc2c1] | 8 |
| 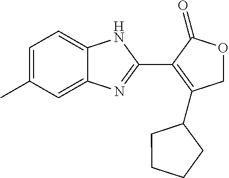 [SMILES: Cc1ccc2[nH]c(C3=C(C4CCCC4)COC3=O)nc2c1] | 9 |
| 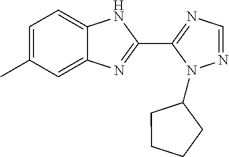 [SMILES: Cc1ccc2[nH]c(-c3ncnn3C3CCCC3)nc2c1] | 10 |
| 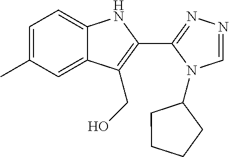 [SMILES: Cc1ccc2[nH]c(-c3nncn3C3CCCC3)c(CO)c2c1] | 11 |
| 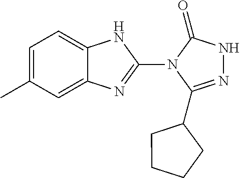 [SMILES: Cc1ccc2[nH]c(-n3c(C4CCCC4)n[nH]c3=O)nc2c1] | 12 |
| 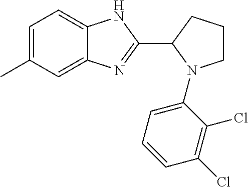 [SMILES: Cc1ccc2[nH]c(C3CCCN3c3cccc(Cl)c3Cl)nc2c1] | 13 |
| 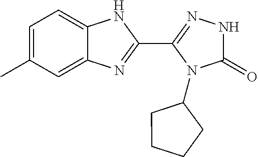 [SMILES: Cc1ccc2[nH]c(-c3n[nH]c(=O)n3C3CCCC3)nc2c1] | 14 |
| 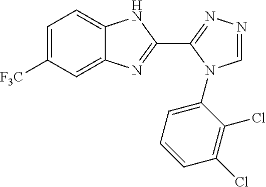 [SMILES: FC(F)(F)c1ccc2[nH]c(-c3nncn3-c3cccc(Cl)c3Cl)nc2c1] | 15 |
| 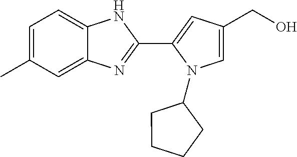 [SMILES: Cc1ccc2[nH]c(-c3cc(CO)cn3C3CCCC3)nc2c1] | 16 |
| 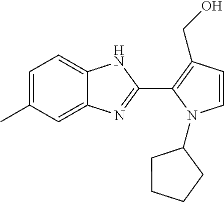 [SMILES: Cc1ccc2[nH]c(-c3c(CO)ccn3C3CCCC3)nc2c1] | 17 |
| 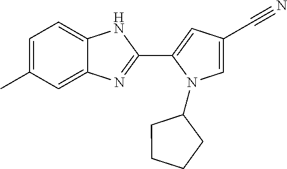 [SMILES: Cc1ccc2[nH]c(-c3cc(C#N)cn3C3CCCC3)nc2c1] | 18 |
| 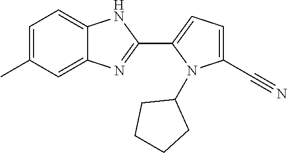 [SMILES: Cc1ccc2[nH]c(-c3ccc(C#N)n3C3CCCC3)nc2c1] | 19 |
| 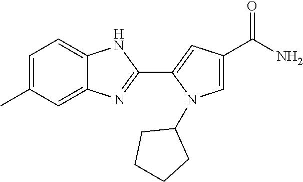 [SMILES: Cc1ccc2[nH]c(-c3cc(C(N)=O)cn3C3CCCC3)nc2c1] | 20 |
| 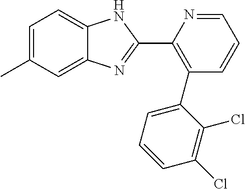 [SMILES: Cc1ccc2[nH]c(-c3ncccc3-c3cccc(Cl)c3Cl)nc2c1] | 21 |
| 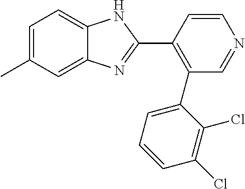 [SMILES: Cc1ccc2[nH]c(-c3ccncc3-c3cccc(Cl)c3Cl)nc2c1] | 22 |
| 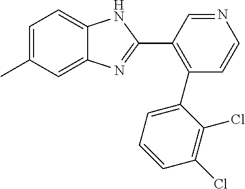 [SMILES: Cc1ccc2[nH]c(-c3cnccc3-c3cccc(Cl)c3Cl)nc2c1] | 23 |
| 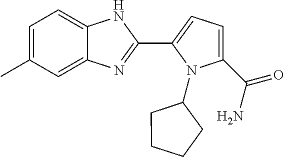 [SMILES: Cc1ccc2[nH]c(-c3ccc(C(N)=O)n3C3CCCC3)nc2c1] | 24 |
| 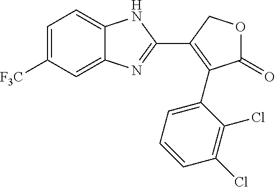 [SMILES: O=C1OCC(c2nc3cc(C(F)(F)F)ccc3[nH]2)=C1c1cccc(Cl)c1Cl] | 25 |
| 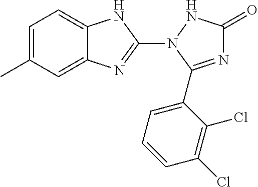 [SMILES: Cc1ccc2[nH]c(-n3[nH]c(=O)nc3-c3cccc(Cl)c3Cl)nc2c1] | 26 |
| 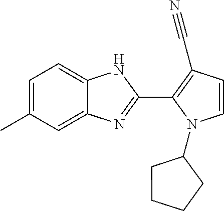 [SMILES: Cc1ccc2[nH]c(-c3c(C#N)ccn3C3CCCC3)nc2c1] | 27 |
| 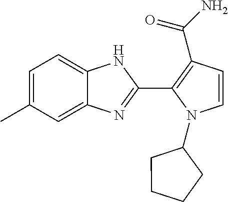 [SMILES: Cc1ccc2[nH]c(-c3c(C(N)=O)ccn3C3CCCC3)nc2c1] | 28 |
| 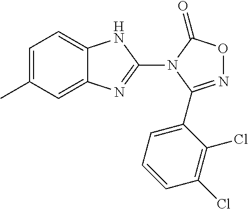 [SMILES: Cc1ccc2[nH]c(-n3c(-c4cccc(Cl)c4Cl)noc3=O)nc2c1] | 29 |
| 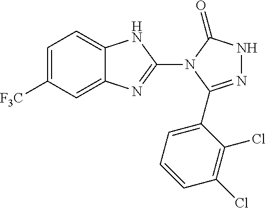 [SMILES: O=c1[nH]nc(-c2cccc(Cl)c2Cl)n1-c1nc2cc(C(F)(F)F)ccc2[nH]1] | 30 |
| 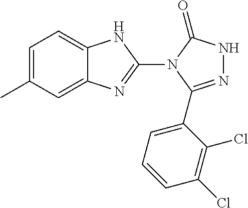 [SMILES: Cc1ccc2[nH]c(-n3c(-c4cccc(Cl)c4Cl)n[nH]c3=O)nc2c1] | 31 |
| 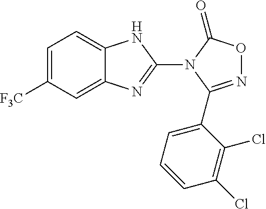 [SMILES: O=c1onc(-c2cccc(Cl)c2Cl)n1-c1nc2cc(C(F)(F)F)ccc2[nH]1] | 32 |
| 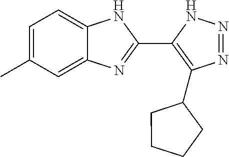 [SMILES: Cc1ccc2[nH]c(-c3[nH]nnc3C3CCCC3)nc2c1] | 33 |
| 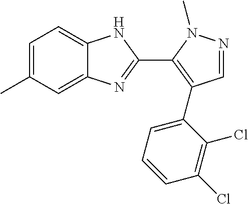 [SMILES: Cc1ccc2[nH]c(-c3c(-c4cccc(Cl)c4Cl)cnn3C)nc2c1] | 34 |
| 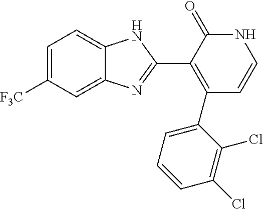 [SMILES: O=c1[nH]ccc(-c2cccc(Cl)c2Cl)c1-c1nc2cc(C(F)(F)F)ccc2[nH]1] | 35 |
| 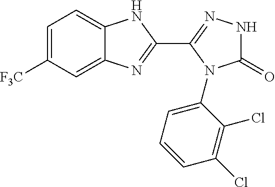 [SMILES: O=c1[nH]nc(-c2nc3cc(C(F)(F)F)ccc3[nH]2)n1-c1cccc(Cl)c1Cl] | 36 |
| 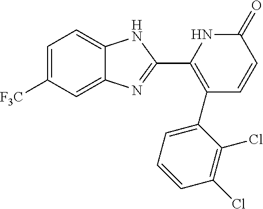 [SMILES: O=c1ccc(-c2cccc(Cl)c2Cl)c(-c2nc3cc(C(F)(F)F)ccc3[nH]2)[nH]1] | 37 |
| 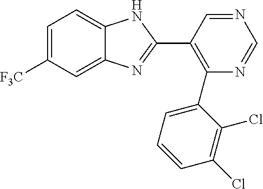 [SMILES: FC(F)(F)c1ccc2[nH]c(-c3cncnc3-c3cccc(Cl)c3Cl)nc2c1] | 38 |
| 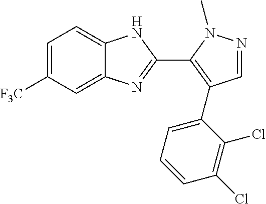 [SMILES: Cn1ncc(-c2cccc(Cl)c2Cl)c1-c1nc2cc(C(F)(F)F)ccc2[nH]1] | 39 |
| 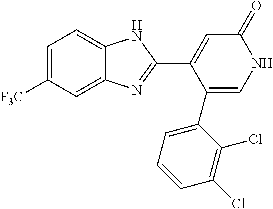 [SMILES: O=c1cc(-c2nc3cc(C(F)(F)F)ccc3[nH]2)c(-c2cccc(Cl)c2Cl)c[nH]1] | 40 |
| 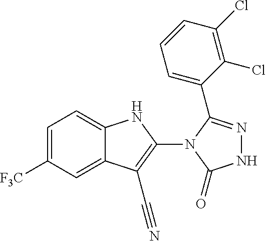 [SMILES: N#Cc1c(-n2c(-c3cccc(Cl)c3Cl)n[nH]c2=O)[nH]c2ccc(C(F)(F)F)cc12] | 41 |
| 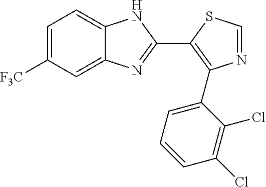 [SMILES: FC(F)(F)c1ccc2[nH]c(-c3scnc3-c3cccc(Cl)c3Cl)nc2c1] | 42 |
| 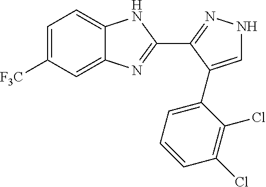 [SMILES: FC(F)(F)c1ccc2[nH]c(-c3n[nH]cc3-c3cccc(Cl)c3Cl)nc2c1] | 43 |
| 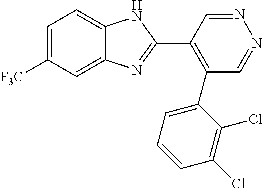 [SMILES: FC(F)(F)c1ccc2[nH]c(-c3cnncc3-c3cccc(Cl)c3Cl)nc2c1] | 44 |
| 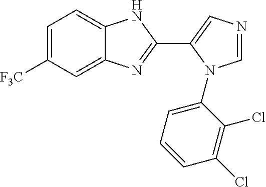 [SMILES: FC(F)(F)c1ccc2[nH]c(-c3cncn3-c3cccc(Cl)c3Cl)nc2c1] | 45 |
| 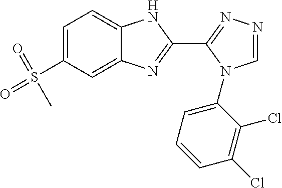 [SMILES: CS(=O)(=O)c1ccc2[nH]c(-c3nncn3-c3cccc(Cl)c3Cl)nc2c1] | 46 |
| 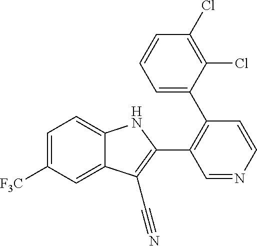 [SMILES: N#Cc1c(-c2cnccc2-c2cccc(Cl)c2Cl)[nH]c2ccc(C(F)(F)F)cc12] | 47 |
| 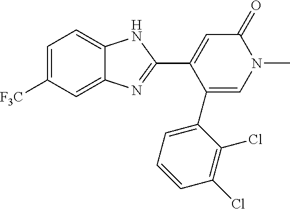 [SMILES: Cn1cc(-c2cccc(Cl)c2Cl)c(-c2nc3cc(C(F)(F)F)ccc3[nH]2)cc1=O] | 48 |
| 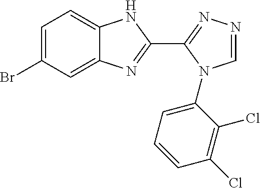 [SMILES: Clc1cccc(-n2cnnc2-c2nc3cc(Br)ccc3[nH]2)c1Cl] | 49 |
| 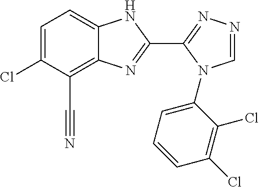 [SMILES: N#Cc1c(Cl)ccc2[nH]c(-c3nncn3-c3cccc(Cl)c3Cl)nc12] | 50 |
| 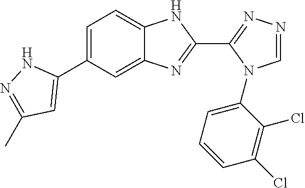 [SMILES: Cc1cc(-c2ccc3[nH]c(-c4nncn4-c4cccc(Cl)c4Cl)nc3c2)[nH]n1] | 51 |
| 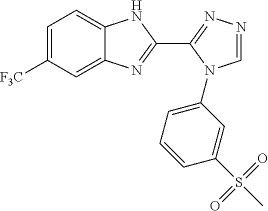 [SMILES: CS(=O)(=O)c1cccc(-n2cnnc2-c2nc3cc(C(F)(F)F)ccc3[nH]2)c1] | 52 |
| 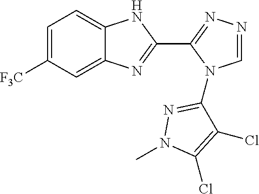 [SMILES: Cn1nc(-n2cnnc2-c2nc3cc(C(F)(F)F)ccc3[nH]2)c(Cl)c1Cl] | 53 |
| 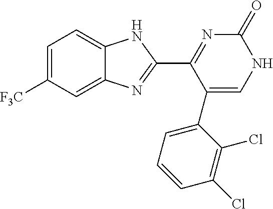 [SMILES: O=c1nc(-c2nc3cc(C(F)(F)F)ccc3[nH]2)c(-c2cccc(Cl)c2Cl)c[nH]1] | 54 |
| 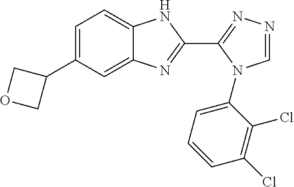 [SMILES: Clc1cccc(-n2cnnc2-c2nc3cc(C4COC4)ccc3[nH]2)c1Cl] | 55 |
| 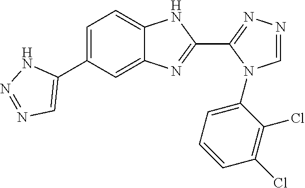 [SMILES: Clc1cccc(-n2cnnc2-c2nc3cc(-c4cnn[nH]4)ccc3[nH]2)c1Cl] | 56 |
| 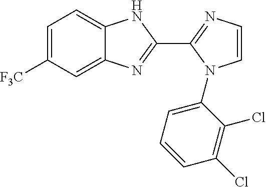 [SMILES: FC(F)(F)c1ccc2[nH]c(-c3nccn3-c3cccc(Cl)c3Cl)nc2c1] | 57 |
| 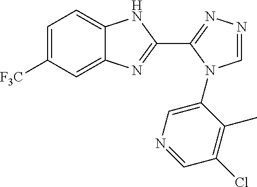 [SMILES: Cc1c(Cl)cncc1-n1cnnc1-c1nc2cc(C(F)(F)F)ccc2[nH]1] | 58 |
| 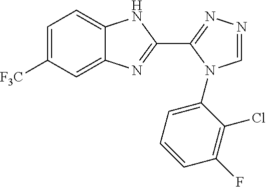 [SMILES: Fc1cccc(-n2cnnc2-c2nc3cc(C(F)(F)F)ccc3[nH]2)c1Cl] | 59 |
| 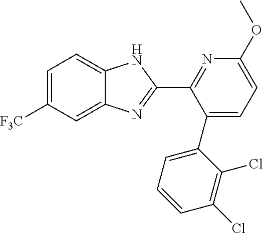 [SMILES: COc1ccc(-c2cccc(Cl)c2Cl)c(-c2nc3cc(C(F)(F)F)ccc3[nH]2)n1] | 60 |
| 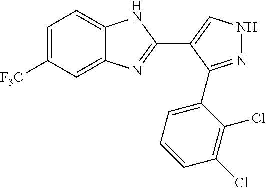 [SMILES: FC(F)(F)c1ccc2[nH]c(-c3c[nH]nc3-c3cccc(Cl)c3Cl)nc2c1] | 61 |
| 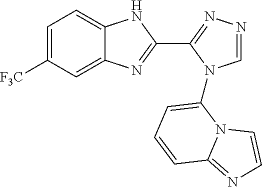 [SMILES: FC(F)(F)c1ccc2[nH]c(-c3nncn3-c3cccc4nccn34)nc2c1] | 62 |
| 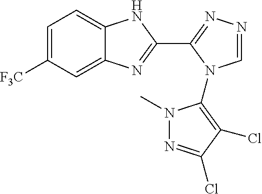 [SMILES: Cn1nc(Cl)c(Cl)c1-n1cnnc1-c1nc2cc(C(F)(F)F)ccc2[nH]1] | 63 |
| 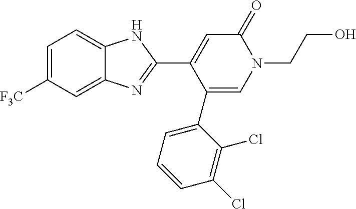 [SMILES: O=c1cc(-c2nc3cc(C(F)(F)F)ccc3[nH]2)c(-c2cccc(Cl)c2Cl)cn1CCO] | 64 |
| 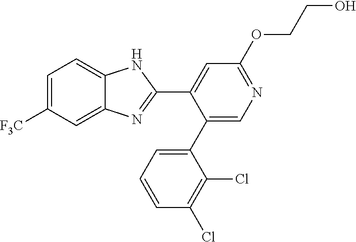 [SMILES: OCCOc1cc(-c2nc3cc(C(F)(F)F)ccc3[nH]2)c(-c2cccc(Cl)c2Cl)cn1] | 65 |
| 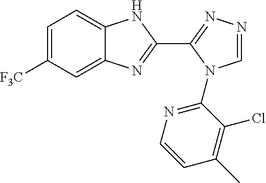 [SMILES: Cc1ccnc(-n2cnnc2-c2nc3cc(C(F)(F)F)ccc3[nH]2)c1Cl] | 66 |
| 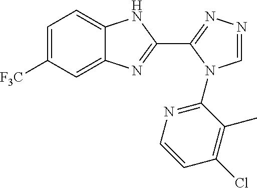 [SMILES: Cc1c(Cl)ccnc1-n1cnnc1-c1nc2cc(C(F)(F)F)ccc2[nH]1] | 67 |
| 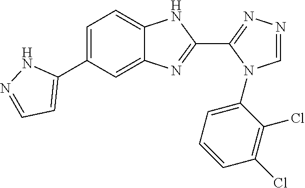 [SMILES: Clc1cccc(-n2cnnc2-c2nc3cc(-c4ccn[nH]4)ccc3[nH]2)c1Cl] | 68 |
| 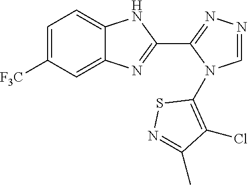 [SMILES: Cc1nsc(-n2cnnc2-c2nc3cc(C(F)(F)F)ccc3[nH]2)c1Cl] | 69 |
| 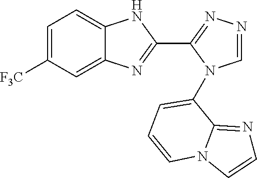 [SMILES: FC(F)(F)c1ccc2[nH]c(-c3nncn3-c3cccn4ccnc34)nc2c1] | 70 |
| 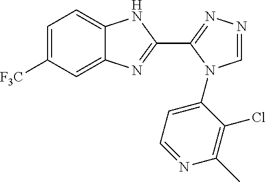 [SMILES: Cc1nccc(-n2cnnc2-c2nc3cc(C(F)(F)F)ccc3[nH]2)c1Cl] | 71 |
| 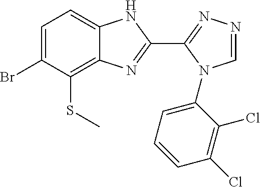 [SMILES: CSc1c(Br)ccc2[nH]c(-c3nncn3-c3cccc(Cl)c3Cl)nc12] | 72 |
| 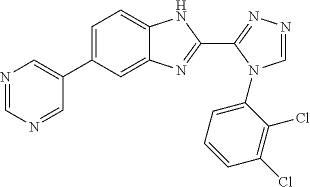 [SMILES: Clc1cccc(-n2cnnc2-c2nc3cc(-c4cncnc4)ccc3[nH]2)c1Cl] | 73 |
| 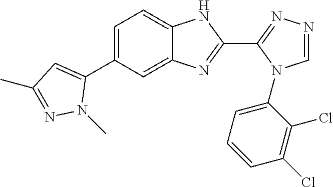 [SMILES: Cc1cc(-c2ccc3[nH]c(-c4nncn4-c4cccc(Cl)c4Cl)nc3c2)n(C)n1] | 74 |
| 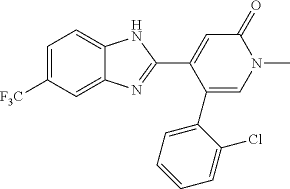 [SMILES: Cn1cc(-c2ccccc2Cl)c(-c2nc3cc(C(F)(F)F)ccc3[nH]2)cc1=O] | 75 |
| 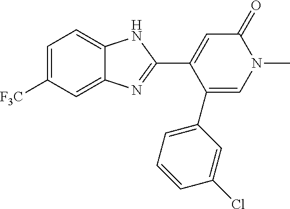 [SMILES: Cn1cc(-c2cccc(Cl)c2)c(-c2nc3cc(C(F)(F)F)ccc3[nH]2)cc1=O] | 76 |
| 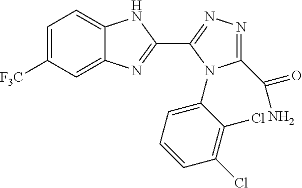 [SMILES: NC(=O)c1nnc(-c2nc3cc(C(F)(F)F)ccc3[nH]2)n1-c1cccc(Cl)c1Cl] | 77 |
| 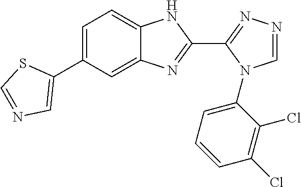 [SMILES: Clc1cccc(-n2cnnc2-c2nc3cc(-c4cncs4)ccc3[nH]2)c1Cl] | 78 |
| 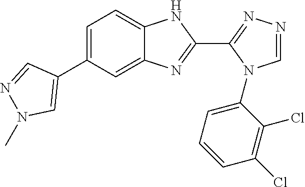 [SMILES: Cn1cc(-c2ccc3[nH]c(-c4nncn4-c4cccc(Cl)c4Cl)nc3c2)cn1] | 79 |
| 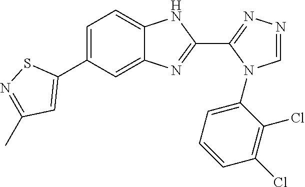 [SMILES: Cc1cc(-c2ccc3[nH]c(-c4nncn4-c4cccc(Cl)c4Cl)nc3c2)sn1] | 80 |
| 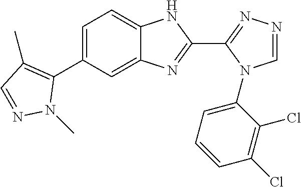 [SMILES: Cc1cnn(C)c1-c1ccc2[nH]c(-c3nncn3-c3cccc(Cl)c3Cl)nc2c1] | 81 |
| 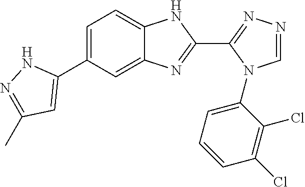 [SMILES: Cc1cc(-c2ccc3[nH]c(-c4nncn4-c4cccc(Cl)c4Cl)nc3c2)[nH]n1] | 82 |
| 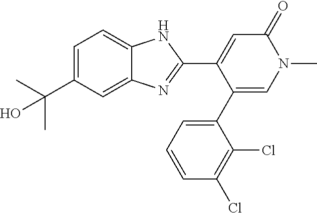 [SMILES: Cn1cc(-c2cccc(Cl)c2Cl)c(-c2nc3cc(C(C)(C)O)ccc3[nH]2)cc1=O] | 83 |
| 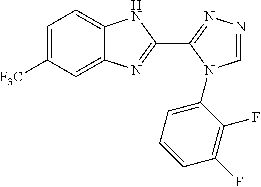 [SMILES: Fc1cccc(-n2cnnc2-c2nc3cc(C(F)(F)F)ccc3[nH]2)c1F] | 84 |
| 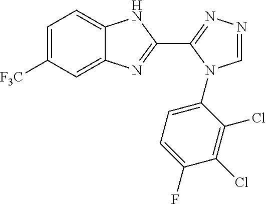 [SMILES: Fc1ccc(-n2cnnc2-c2nc3cc(C(F)(F)F)ccc3[nH]2)c(Cl)c1Cl] | 85 |
| 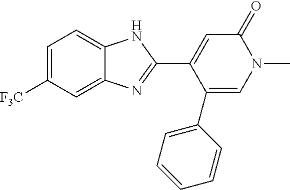 [SMILES: Cn1cc(-c2ccccc2)c(-c2nc3cc(C(F)(F)F)ccc3[nH]2)cc1=O] | 86 |
| 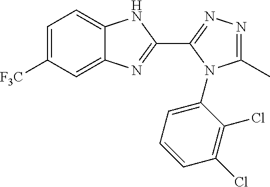 [SMILES: Cc1nnc(-c2nc3cc(C(F)(F)F)ccc3[nH]2)n1-c1cccc(Cl)c1Cl] | 87 |
| 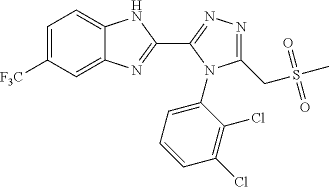 [SMILES: CS(=O)(=O)Cc1nnc(-c2nc3cc(C(F)(F)F)ccc3[nH]2)n1-c1cccc(Cl)c1Cl] | 88 |
| 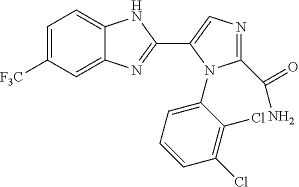 [SMILES: NC(=O)c1ncc(-c2nc3cc(C(F)(F)F)ccc3[nH]2)n1-c1cccc(Cl)c1Cl] | 89 |
| 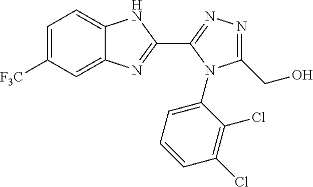 [SMILES: OCc1nnc(-c2nc3cc(C(F)(F)F)ccc3[nH]2)n1-c1cccc(Cl)c1Cl] | 90 |
| 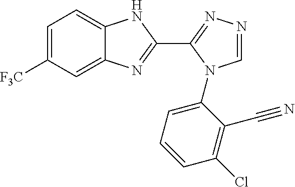 [SMILES: N#Cc1c(Cl)cccc1-n1cnnc1-c1nc2cc(C(F)(F)F)ccc2[nH]1] | 91 |
| 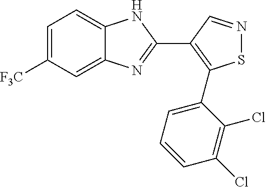 [SMILES: FC(F)(F)c1ccc2[nH]c(-c3cnsc3-c3cccc(Cl)c3Cl)nc2c1] | 92 |
| 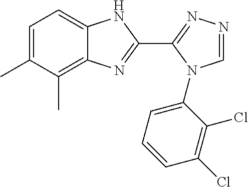 [SMILES: Cc1ccc2[nH]c(-c3nncn3-c3cccc(Cl)c3Cl)nc2c1C] | 93 |
| 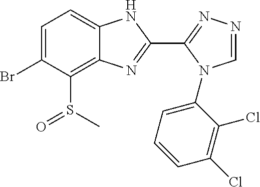 [SMILES: CS(=O)c1c(Br)ccc2[nH]c(-c3nncn3-c3cccc(Cl)c3Cl)nc12] | 94 |
| 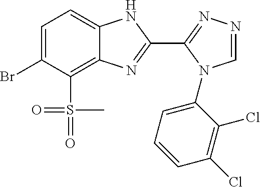 [SMILES: CS(=O)(=O)c1c(Br)ccc2[nH]c(-c3nncn3-c3cccc(Cl)c3Cl)nc12] | 95 |
| 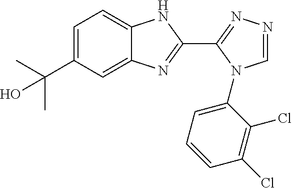 [SMILES: CC(C)(O)c1ccc2[nH]c(-c3nncn3-c3cccc(Cl)c3Cl)nc2c1] | 96 |
| 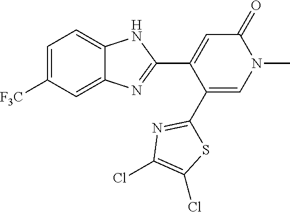 [SMILES: Cn1cc(-c2nc(Cl)c(Cl)s2)c(-c2nc3cc(C(F)(F)F)ccc3[nH]2)cc1=O] | 97 |
| 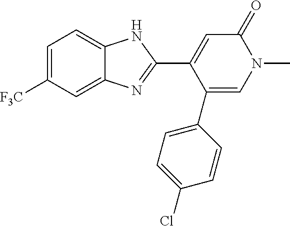 [SMILES: Cn1cc(-c2ccc(Cl)cc2)c(-c2nc3cc(C(F)(F)F)ccc3[nH]2)cc1=O] | 98 |
| 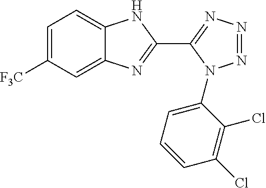 [SMILES: FC(F)(F)c1ccc2[nH]c(-c3nnnn3-c3cccc(Cl)c3Cl)nc2c1] | 99 |
| 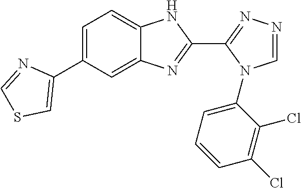 [SMILES: Clc1cccc(-n2cnnc2-c2nc3cc(-c4cscn4)ccc3[nH]2)c1Cl] | 100 |
| 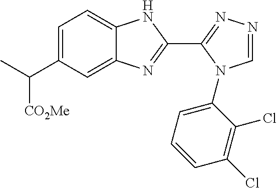 [SMILES: COC(=O)C(C)c1ccc2[nH]c(-c3nncn3-c3cccc(Cl)c3Cl)nc2c1] | 101 |
| 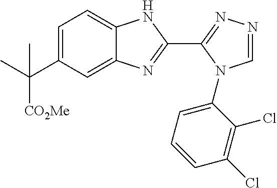 [SMILES: COC(=O)C(C)(C)c1ccc2[nH]c(-c3nncn3-c3cccc(Cl)c3Cl)nc2c1] | 102 |
| 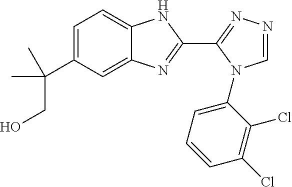 [SMILES: CC(C)(CO)c1ccc2[nH]c(-c3nncn3-c3cccc(Cl)c3Cl)nc2c1] | 103 |
| 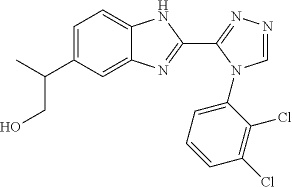 [SMILES: CC(CO)c1ccc2[nH]c(-c3nncn3-c3cccc(Cl)c3Cl)nc2c1] | 104 |
| 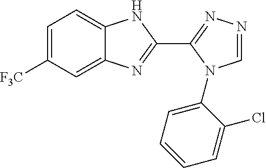 [SMILES: FC(F)(F)c1ccc2[nH]c(-c3nncn3-c3ccccc3Cl)nc2c1] | 105 |
| 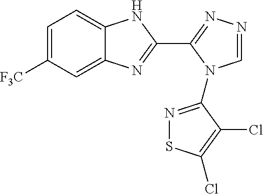 [SMILES: FC(F)(F)c1ccc2[nH]c(-c3nncn3-c3nsc(Cl)c3Cl)nc2c1] | 106 |
| 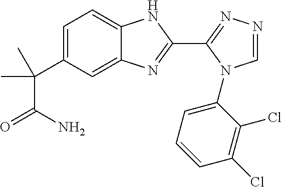 [SMILES: CC(C)(C(N)=O)c1ccc2[nH]c(-c3nncn3-c3cccc(Cl)c3Cl)nc2c1] | 107 |
| 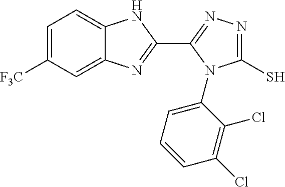 [SMILES: FC(F)(F)c1ccc2[nH]c(-c3nnc(S)n3-c3cccc(Cl)c3Cl)nc2c1] | 108 |
| 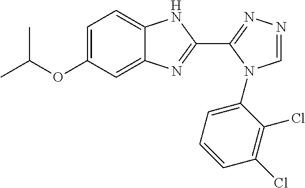 [SMILES: CC(C)Oc1ccc2[nH]c(-c3nncn3-c3cccc(Cl)c3Cl)nc2c1] | 109 |
| 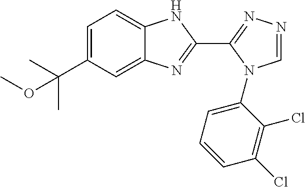 [SMILES: COC(C)(C)c1ccc2[nH]c(-c3nncn3-c3cccc(Cl)c3Cl)nc2c1] | 110 |
| 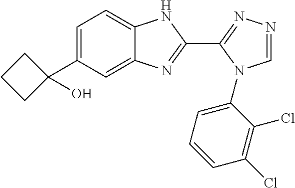 [SMILES: OC1(c2ccc3[nH]c(-c4nncn4-c4cccc(Cl)c4Cl)nc3c2)CCC1] | 111 |
| 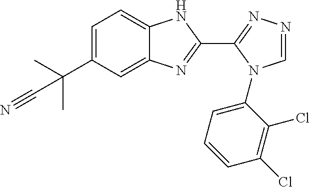 [SMILES: CC(C)(C#N)c1ccc2[nH]c(-c3nncn3-c3cccc(Cl)c3Cl)nc2c1] | 112 |
| 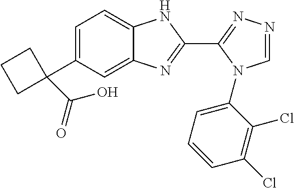 [SMILES: O=C(O)C1(c2ccc3[nH]c(-c4nncn4-c4cccc(Cl)c4Cl)nc3c2)CCC1] | 113 |
| 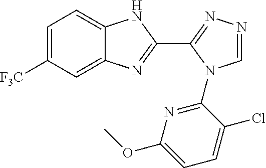 [SMILES: COc1ccc(Cl)c(-n2cnnc2-c2nc3cc(C(F)(F)F)ccc3[nH]2)n1] | 114 |
| 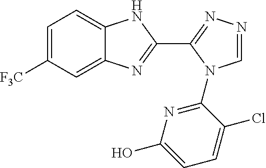 [SMILES: Oc1ccc(Cl)c(-n2cnnc2-c2nc3cc(C(F)(F)F)ccc3[nH]2)n1] | 115 |
| 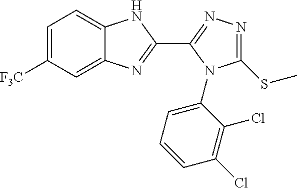 [SMILES: CSc1nnc(-c2nc3cc(C(F)(F)F)ccc3[nH]2)n1-c1cccc(Cl)c1Cl] | 116 |
| 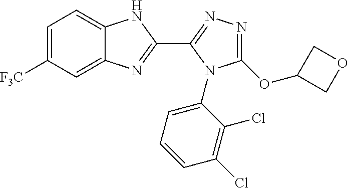 [SMILES: FC(F)(F)c1ccc2[nH]c(-c3nnc(OC4COC4)n3-c3cccc(Cl)c3Cl)nc2c1] | 117 |
| 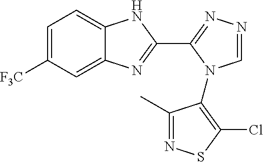 [SMILES: Cc1nsc(Cl)c1-n1cnnc1-c1nc2cc(C(F)(F)F)ccc2[nH]1] | 118 |
| 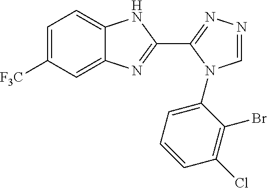 [SMILES: FC(F)(F)c1ccc2[nH]c(-c3nncn3-c3cccc(Cl)c3Br)nc2c1] | 119 |
| 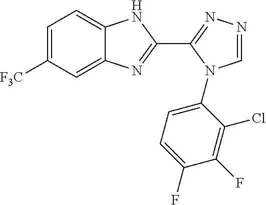 [SMILES: Fc1ccc(-n2cnnc2-c2nc3cc(C(F)(F)F)ccc3[nH]2)c(Cl)c1F] | 120 |
| 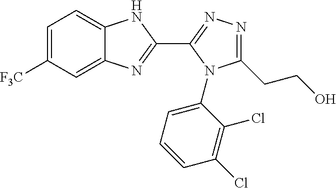 [SMILES: OCCc1nnc(-c2nc3cc(C(F)(F)F)ccc3[nH]2)n1-c1cccc(Cl)c1Cl] | 121 |
| 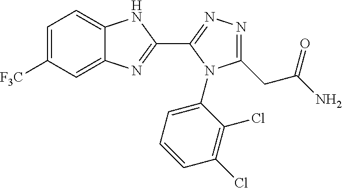 [SMILES: NC(=O)Cc1nnc(-c2nc3cc(C(F)(F)F)ccc3[nH]2)n1-c1cccc(Cl)c1Cl] | 122 |
| 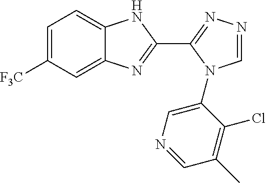 [SMILES: Cc1cncc(-n2cnnc2-c2nc3cc(C(F)(F)F)ccc3[nH]2)c1Cl] | 123 |
| 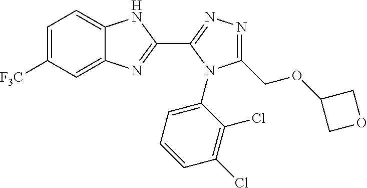 [SMILES: FC(F)(F)c1ccc2[nH]c(-c3nnc(COC4COC4)n3-c3cccc(Cl)c3Cl)nc2c1] | 124 |
| 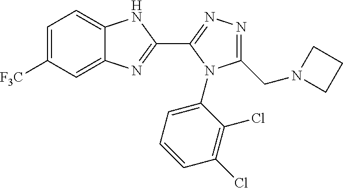 [SMILES: FC(F)(F)c1ccc2[nH]c(-c3nnc(CN4CCC4)n3-c3cccc(Cl)c3Cl)nc2c1] | 125 |
| 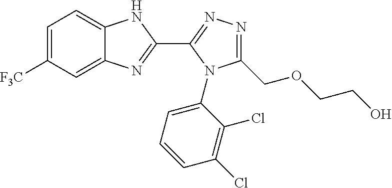 [SMILES: OCCOCc1nnc(-c2nc3cc(C(F)(F)F)ccc3[nH]2)n1-c1cccc(Cl)c1Cl] | 126 |
| 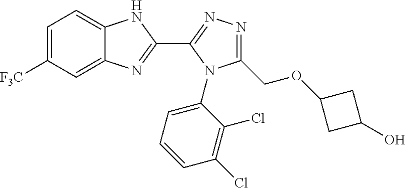 [SMILES: OC1CC(OCc2nnc(-c3nc4cc(C(F)(F)F)ccc4[nH]3)n2-c2cccc(Cl)c2Cl)C1] | 127 |
| 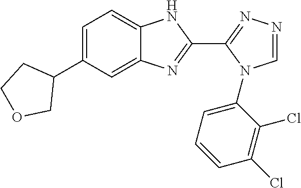 [SMILES: Clc1cccc(-n2cnnc2-c2nc3cc(C4CCOC4)ccc3[nH]2)c1Cl] | 128 |
| 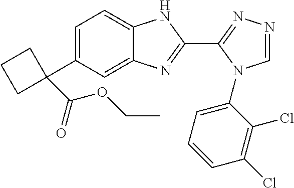 [SMILES: CCOC(=O)C1(c2ccc3[nH]c(-c4nncn4-c4cccc(Cl)c4Cl)nc3c2)CCC1] | 129 |
| 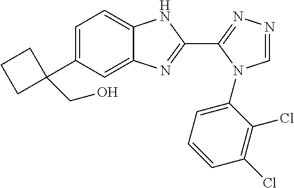 [SMILES: OCC1(c2ccc3[nH]c(-c4nncn4-c4cccc(Cl)c4Cl)nc3c2)CCC1] | 130 |
| 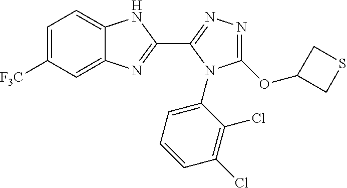 [SMILES: FC(F)(F)c1ccc2[nH]c(-c3nnc(OC4CSC4)n3-c3cccc(Cl)c3Cl)nc2c1] | 131 |
| 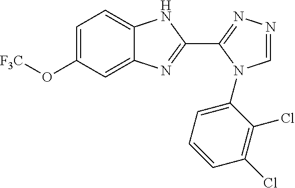 [SMILES: FC(F)(F)Oc1ccc2[nH]c(-c3nncn3-c3cccc(Cl)c3Cl)nc2c1] | 132 |
| 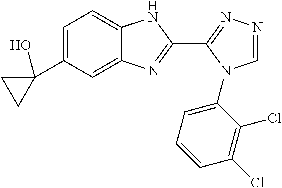 [SMILES: OC1(c2ccc3[nH]c(-c4nncn4-c4cccc(Cl)c4Cl)nc3c2)CC1] | 133 |
| 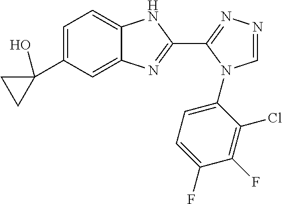 [SMILES: OC1(c2ccc3[nH]c(-c4nncn4-c4ccc(F)c(F)c4Cl)nc3c2)CC1] | 134 |
| 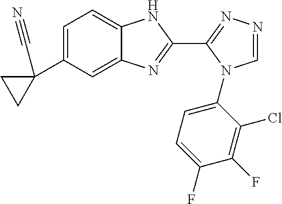 [SMILES: N#CC1(c2ccc3[nH]c(-c4nncn4-c4ccc(F)c(F)c4Cl)nc3c2)CC1] | 135 |
| 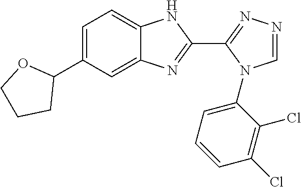 [SMILES: Clc1cccc(-n2cnnc2-c2nc3cc(C4CCCO4)ccc3[nH]2)c1Cl] | 136 |
| 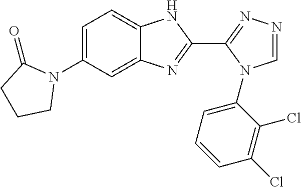 [SMILES: O=C1CCCN1c1ccc2[nH]c(-c3nncn3-c3cccc(Cl)c3Cl)nc2c1] | 137 |
| 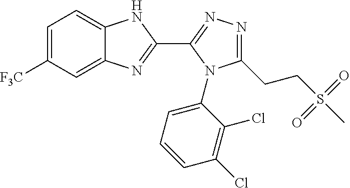 [SMILES: CS(=O)(=O)CCc1nnc(-c2nc3cc(C(F)(F)F)ccc3[nH]2)n1-c1cccc(Cl)c1Cl] | 138 |
| 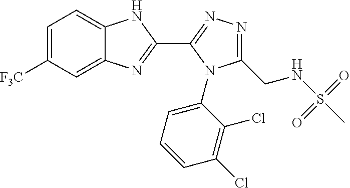 [SMILES: CS(=O)(=O)NCc1nnc(-c2nc3cc(C(F)(F)F)ccc3[nH]2)n1-c1cccc(Cl)c1Cl] | 139 |
| 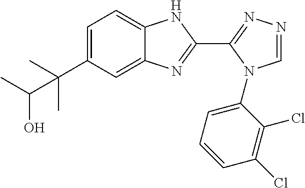 [SMILES: CC(O)C(C)(C)c1ccc2[nH]c(-c3nncn3-c3cccc(Cl)c3Cl)nc2c1] | 140 |
| 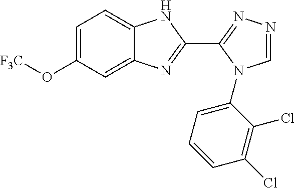 [SMILES: FC(F)(F)Oc1ccc2[nH]c(-c3nncn3-c3cccc(Cl)c3Cl)nc2c1] | 141 |
| 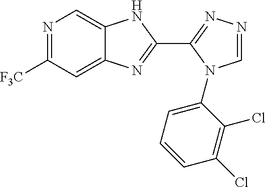 [SMILES: FC(F)(F)c1cc2nc(-c3nncn3-c3cccc(Cl)c3Cl)[nH]c2cn1] | 142 |
| 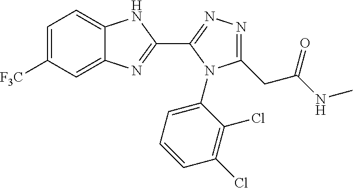 [SMILES: CNC(=O)Cc1nnc(-c2nc3cc(C(F)(F)F)ccc3[nH]2)n1-c1cccc(Cl)c1Cl] | 143 |
| 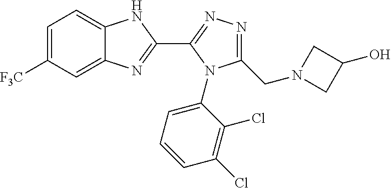 [SMILES: OC1CN(Cc2nnc(-c3nc4cc(C(F)(F)F)ccc4[nH]3)n2-c2cccc(Cl)c2Cl)C1] | 144 |
| 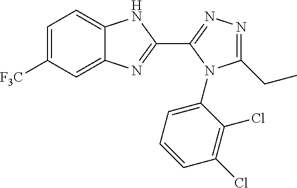 [SMILES: CCc1nnc(-c2nc3cc(C(F)(F)F)ccc3[nH]2)n1-c1cccc(Cl)c1Cl] | 145 |
| 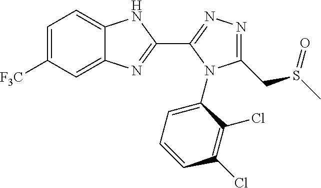 | 146 |
| 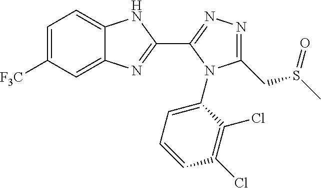 | 147 |
| 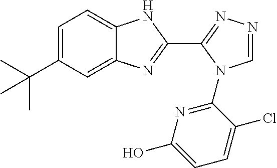 [SMILES: CC(C)(C)c1ccc2[nH]c(-c3nncn3-c3nc(O)ccc3Cl)nc2c1] | 148 |
| 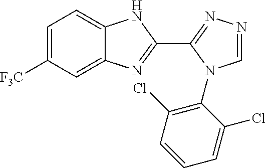 [SMILES: FC(F)(F)c1ccc2[nH]c(-c3nncn3-c3c(Cl)cccc3Cl)nc2c1] | 149 |
| 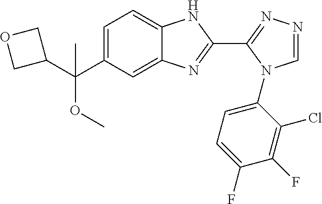 [SMILES: COC(C)(c1ccc2[nH]c(-c3nncn3-c3ccc(F)c(F)c3Cl)nc2c1)C1COC1] | 150 |
| 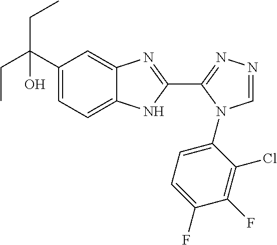 [SMILES: CCC(O)(CC)c1ccc2[nH]c(-c3nncn3-c3ccc(F)c(F)c3Cl)nc2c1] | 151 |
| 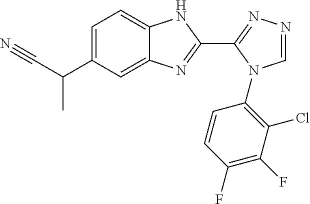 [SMILES: CC(C#N)c1ccc2[nH]c(-c3nncn3-c3ccc(F)c(F)c3Cl)nc2c1] | 152 |
| 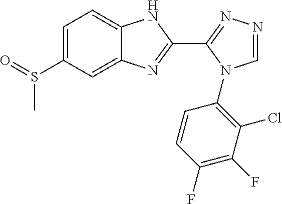 [SMILES: CS(=O)c1ccc2[nH]c(-c3nncn3-c3ccc(F)c(F)c3Cl)nc2c1] | 153 |
| 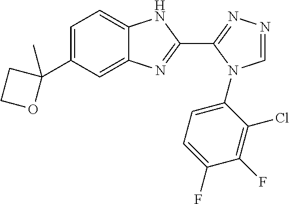 [SMILES: CC1(c2ccc3[nH]c(-c4nncn4-c4ccc(F)c(F)c4Cl)nc3c2)CCO1] | 154 |
| 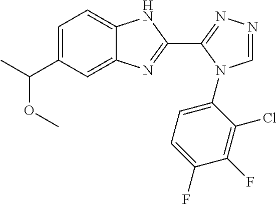 [SMILES: COC(C)c1ccc2[nH]c(-c3nncn3-c3ccc(F)c(F)c3Cl)nc2c1] | 155 |
| 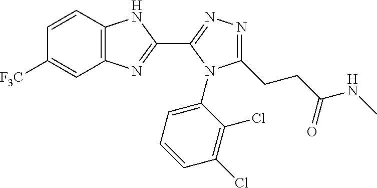 [SMILES: CNC(=O)CCc1nnc(-c2nc3cc(C(F)(F)F)ccc3[nH]2)n1-c1cccc(Cl)c1Cl] | 156 |
| 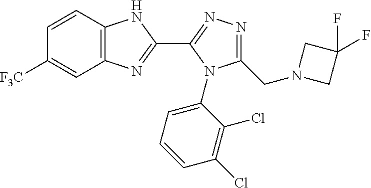 [SMILES: FC1(F)CN(Cc2nnc(-c3nc4cc(C(F)(F)F)ccc4[nH]3)n2-c2cccc(Cl)c2Cl)C1] | 157 |
| 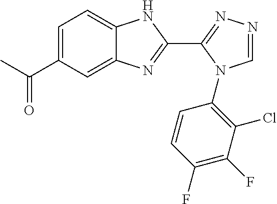 [SMILES: CC(=O)c1ccc2[nH]c(-c3nncn3-c3ccc(F)c(F)c3Cl)nc2c1] | 158 |
| 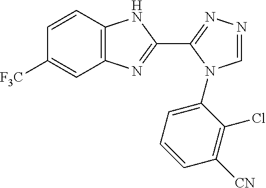 [SMILES: N#Cc1cccc(-n2cnnc2-c2nc3cc(C(F)(F)F)ccc3[nH]2)c1Cl] | 159 |
| 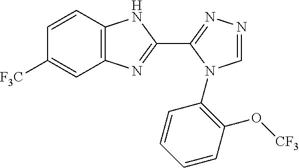 [SMILES: FC(F)(F)Oc1ccccc1-n1cnnc1-c1nc2cc(C(F)(F)F)ccc2[nH]1] | 160 |
| 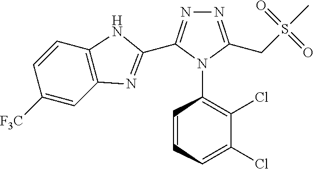 | 161 |
| 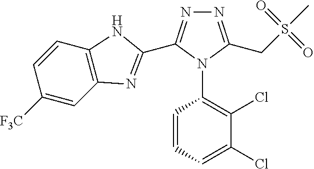 | 162 |
| 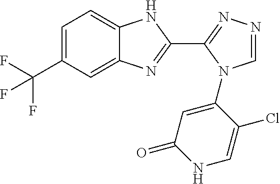 [SMILES: O=c1cc(-n2cnnc2-c2nc3cc(C(F)(F)F)ccc3[nH]2)c(Cl)c[nH]1] | 163 |
| 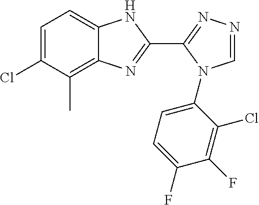 [SMILES: Cc1c(Cl)ccc2[nH]c(-c3nncn3-c3ccc(F)c(F)c3Cl)nc12] | 164 |
| 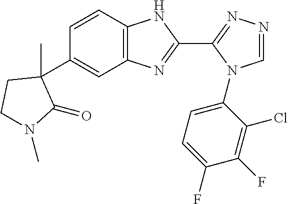 [SMILES: CN1CCC(C)(c2ccc3[nH]c(-c4nncn4-c4ccc(F)c(F)c4Cl)nc3c2)C1=O] | 165 |
| 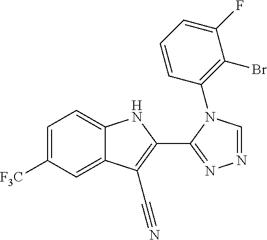 [SMILES: N#Cc1c(-c2nncn2-c2cccc(F)c2Br)[nH]c2ccc(C(F)(F)F)cc12] | 166 |
| 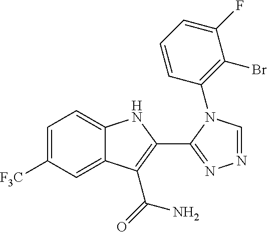 [SMILES: NC(=O)c1c(-c2nncn2-c2cccc(F)c2Br)[nH]c2ccc(C(F)(F)F)cc12] | 167 |
| 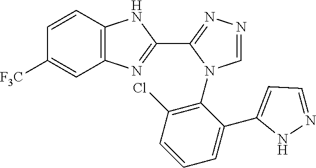 [SMILES: FC(F)(F)c1ccc2[nH]c(-c3nncn3-c3c(Cl)cccc3-c3ccn[nH]3)nc2c1] | 168 |
| 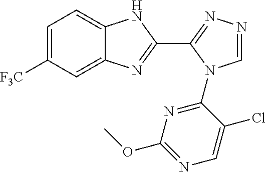 [SMILES: COc1ncc(Cl)c(-n2cnnc2-c2nc3cc(C(F)(F)F)ccc3[nH]2)n1] | 169 |
| 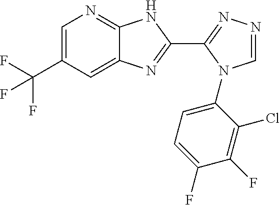 [SMILES: Fc1ccc(-n2cnnc2-c2nc3cc(C(F)(F)F)cnc3[nH]2)c(Cl)c1F] | 170 |
| 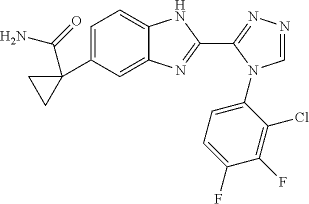 [SMILES: NC(=O)C1(c2ccc3[nH]c(-c4nncn4-c4ccc(F)c(F)c4Cl)nc3c2)CC1] | 171 |
| 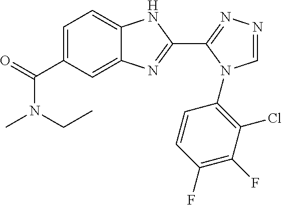 [SMILES: CCN(C)C(=O)c1ccc2[nH]c(-c3nncn3-c3ccc(F)c(F)c3Cl)nc2c1] | 172 |
| 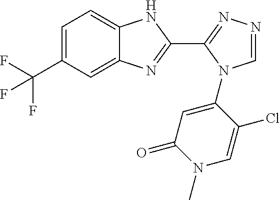 [SMILES: Cn1cc(Cl)c(-n2cnnc2-c2nc3cc(C(F)(F)F)ccc3[nH]2)cc1=O] | 173 |
| 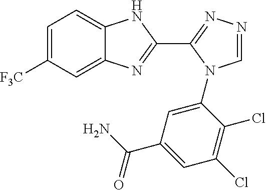 [SMILES: NC(=O)c1cc(Cl)c(Cl)c(-n2cnnc2-c2nc3cc(C(F)(F)F)ccc3[nH]2)c1] | 174 |
| 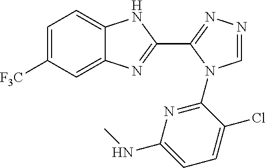 [SMILES: CNc1ccc(Cl)c(-n2cnnc2-c2nc3cc(C(F)(F)F)ccc3[nH]2)n1] | 175 |
| 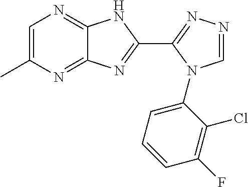 [SMILES: Cc1cnc2[nH]c(-c3nncn3-c3cccc(F)c3Cl)nc2n1] | 176 |
| 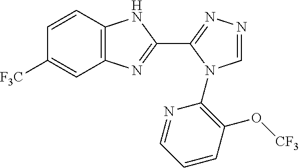 [SMILES: FC(F)(F)Oc1cccnc1-n1cnnc1-c1nc2cc(C(F)(F)F)ccc2[nH]1] | 177 |
| 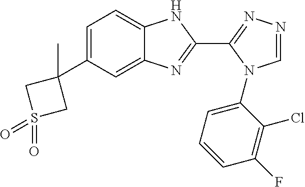 [SMILES: CC1(c2ccc3[nH]c(-c4nncn4-c4cccc(F)c4Cl)nc3c2)CS(=O)(=O)C1] | 178 |
| 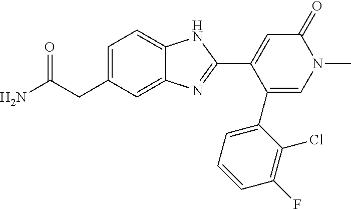 [SMILES: Cn1cc(-c2cccc(F)c2Cl)c(-c2nc3cc(CC(N)=O)ccc3[nH]2)cc1=O] | 179 |
| 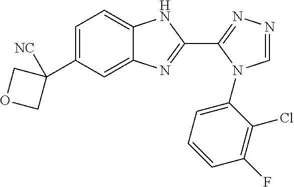 [SMILES: N#CC1(c2ccc3[nH]c(-c4nncn4-c4cccc(F)c4Cl)nc3c2)COC1] | 180 |
| 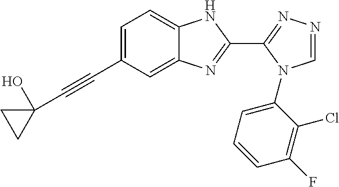 [SMILES: OC1(C#Cc2ccc3[nH]c(-c4nncn4-c4cccc(F)c4Cl)nc3c2)CC1] | 181 |
| 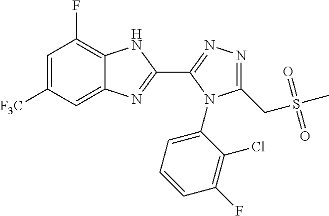 [SMILES: CS(=O)(=O)Cc1nnc(-c2nc3cc(C(F)(F)F)cc(F)c3[nH]2)n1-c1cccc(F)c1Cl] | 182 |
| 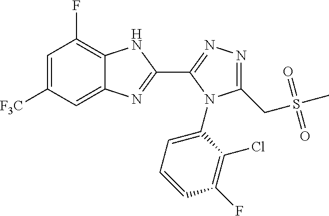 | 183 |
| 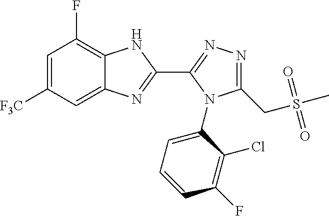 | 184 |
| 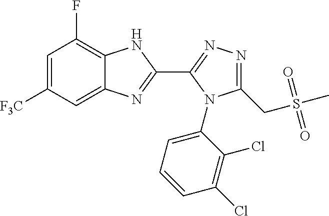 [SMILES: CS(=O)(=O)Cc1nnc(-c2nc3cc(C(F)(F)F)cc(F)c3[nH]2)n1-c1cccc(Cl)c1Cl] | 185 |
| 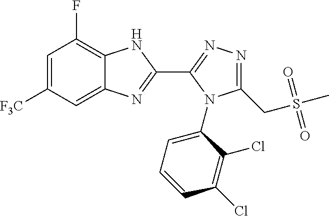 | 186 |
| 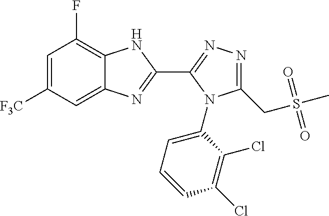 | 187 |
| 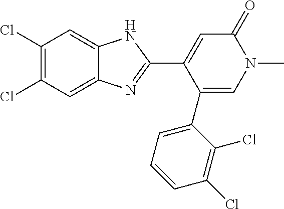 [SMILES: Cn1cc(-c2cccc(Cl)c2Cl)c(-c2nc3cc(Cl)c(Cl)cc3[nH]2)cc1=O] | 188 |
| 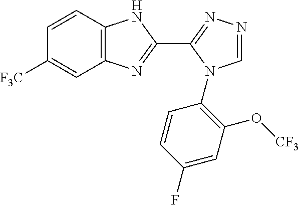 [SMILES: Fc1ccc(-n2cnnc2-c2nc3cc(C(F)(F)F)ccc3[nH]2)c(OC(F)(F)F)c1] | 189 |
| 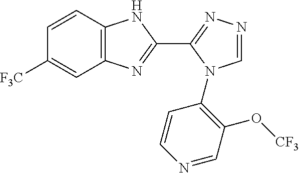 [SMILES: FC(F)(F)Oc1cnccc1-n1cnnc1-c1nc2cc(C(F)(F)F)ccc2[nH]1] | 190 |
| 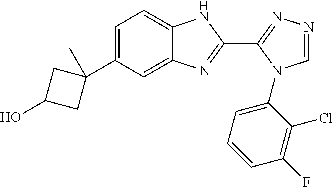 [SMILES: CC1(c2ccc3[nH]c(-c4nncn4-c4cccc(F)c4Cl)nc3c2)CC(O)C1] | 191 |
| 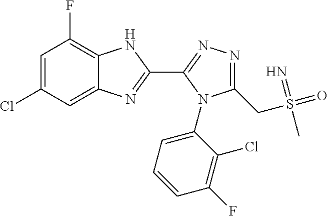 [SMILES: CS(=N)(=O)Cc1nnc(-c2nc3cc(Cl)cc(F)c3[nH]2)n1-c1cccc(F)c1Cl] | 192 |
| 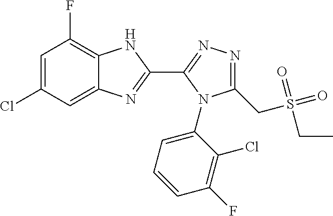 [SMILES: CCS(=O)(=O)Cc1nnc(-c2nc3cc(Cl)cc(F)c3[nH]2)n1-c1cccc(F)c1Cl] | 193 |
| 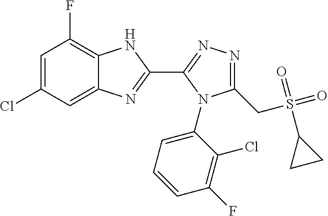 [SMILES: O=S(=O)(Cc1nnc(-c2nc3cc(Cl)cc(F)c3[nH]2)n1-c1cccc(F)c1Cl)C1CC1] | 194 |
| 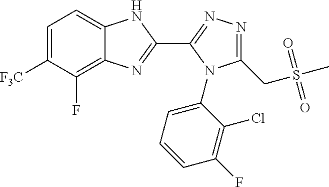 [SMILES: CS(=O)(=O)Cc1nnc(-c2nc3c(F)c(C(F)(F)F)ccc3[nH]2)n1-c1cccc(F)c1Cl] | 195 |
| 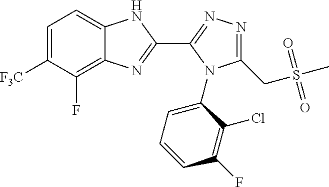 | 196 |
| 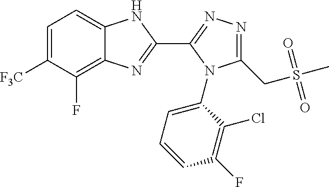 | 197 |
| 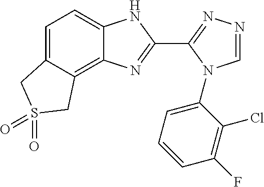 [SMILES: O=S1(=O)Cc2ccc3[nH]c(-c4nncn4-c4cccc(F)c4Cl)nc3c2C1] | 198 |
| 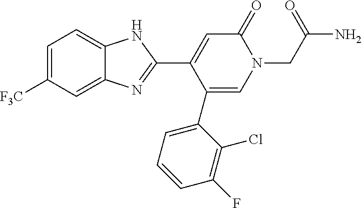 [SMILES: NC(=O)Cn1cc(-c2cccc(F)c2Cl)c(-c2nc3cc(C(F)(F)F)ccc3[nH]2)cc1=O] | 199 |
| 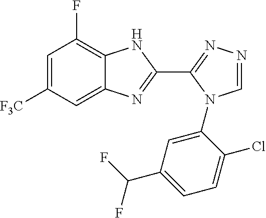 [SMILES: Fc1cc(C(F)(F)F)cc2nc(-c3nncn3-c3cc(C(F)F)ccc3Cl)[nH]c12] | 200 |
| 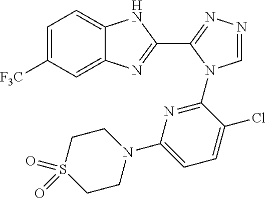 [SMILES: O=S1(=O)CCN(c2ccc(Cl)c(-n3cnnc3-c3nc4cc(C(F)(F)F)ccc4[nH]3)n2)CC1] | 201 |
| 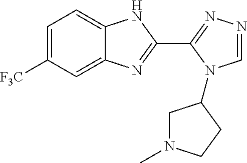 [SMILES: CN1CCC(n2cnnc2-c2nc3cc(C(F)(F)F)ccc3[nH]2)C1] | 202 |
| 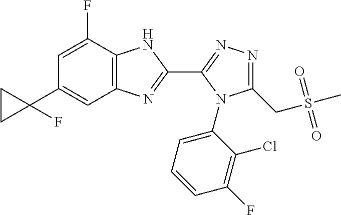 [SMILES: CS(=O)(=O)Cc1nnc(-c2nc3cc(C4(F)CC4)cc(F)c3[nH]2)n1-c1cccc(F)c1Cl] | 203 |
| 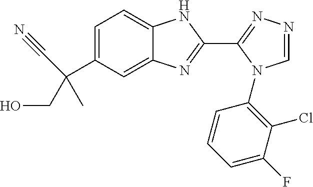 [SMILES: CC(C#N)(CO)c1ccc2[nH]c(-c3nncn3-c3cccc(F)c3Cl)nc2c1] | 204 |
| 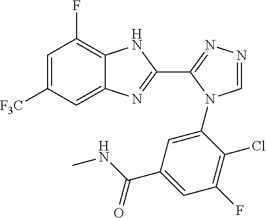 [SMILES: CNC(=O)c1cc(F)c(Cl)c(-n2cnnc2-c2nc3cc(C(F)(F)F)cc(F)c3[nH]2)c1] | 205 |
| 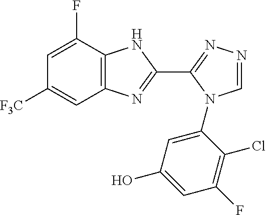 [SMILES: Oc1cc(F)c(Cl)c(-n2cnnc2-c2nc3cc(C(F)(F)F)cc(F)c3[nH]2)c1] | 206 |
| 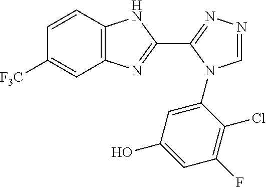 [SMILES: Oc1cc(F)c(Cl)c(-n2cnnc2-c2nc3cc(C(F)(F)F)ccc3[nH]2)c1] | 207 |
| 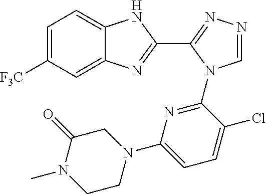 [SMILES: CN1CCN(c2ccc(Cl)c(-n3cnnc3-c3nc4cc(C(F)(F)F)ccc4[nH]3)n2)CC1=O] | 208 |
| 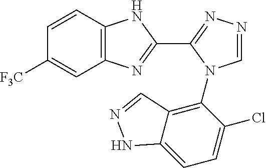 [SMILES: FC(F)(F)c1ccc2[nH]c(-c3nncn3-c3c(Cl)ccc4[nH]ncc34)nc2c1] | 209 |
| 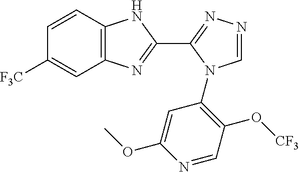 [SMILES: COc1cc(-n2cnnc2-c2nc3cc(C(F)(F)F)ccc3[nH]2)c(OC(F)(F)F)cn1] | 210 |
| 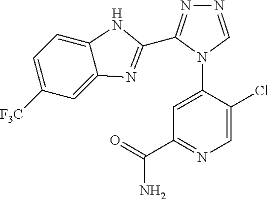 [SMILES: NC(=O)c1cc(-n2cnnc2-c2nc3cc(C(F)(F)F)ccc3[nH]2)c(Cl)cn1] | 211 |
| 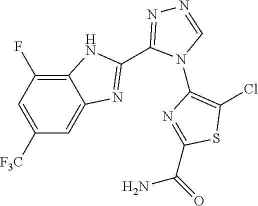 [SMILES: NC(=O)c1nc(-n2cnnc2-c2nc3cc(C(F)(F)F)cc(F)c3[nH]2)c(Cl)s1] | 212 |
| 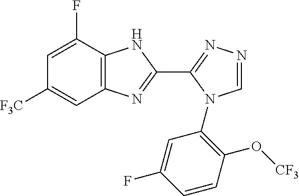 [SMILES: Fc1ccc(OC(F)(F)F)c(-n2cnnc2-c2nc3cc(C(F)(F)F)cc(F)c3[nH]2)c1] | 213 |
| 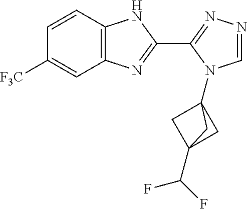 [SMILES: FC(F)C12CC(n3cnnc3-c3nc4cc(C(F)(F)F)ccc4[nH]3)(C1)C2] | 214 |
| 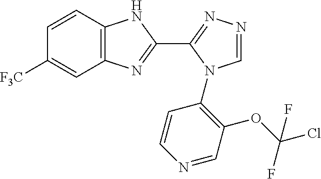 [SMILES: FC(F)(Cl)Oc1cnccc1-n1cnnc1-c1nc2cc(C(F)(F)F)ccc2[nH]1] | 215 |
| 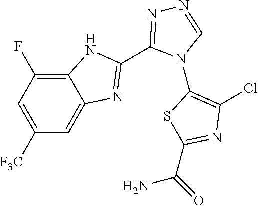 [SMILES: NC(=O)c1nc(Cl)c(-n2cnnc2-c2nc3cc(C(F)(F)F)cc(F)c3[nH]2)s1] | 216 |
| 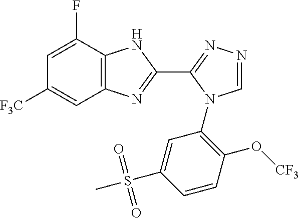 [SMILES: CS(=O)(=O)c1ccc(OC(F)(F)F)c(-n2cnnc2-c2nc3cc(C(F)(F)F)cc(F)c3[nH]2)c1] | 217 |
| 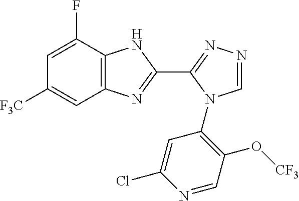 [SMILES: Fc1cc(C(F)(F)F)cc2nc(-c3nncn3-c3cc(Cl)ncc3OC(F)(F)F)[nH]c12] | 218 |
| 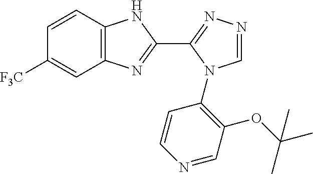 [SMILES: CC(C)(C)Oc1cnccc1-n1cnnc1-c1nc2cc(C(F)(F)F)ccc2[nH]1] | 219 |
| 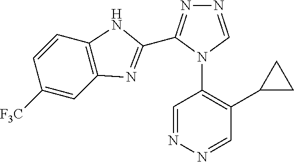 [SMILES: FC(F)(F)c1ccc2[nH]c(-c3nncn3-c3cnncc3C3CC3)nc2c1] | 220 |
| 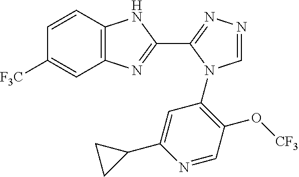 [SMILES: FC(F)(F)Oc1cnc(C2CC2)cc1-n1cnnc1-c1nc2cc(C(F)(F)F)ccc2[nH]1] | 221 |
| 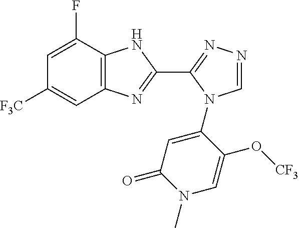 [SMILES: Cn1cc(OC(F)(F)F)c(-n2cnnc2-c2nc3cc(C(F)(F)F)cc(F)c3[nH]2)cc1=O] | 222 |
| 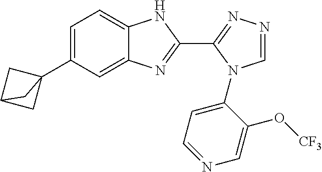 [SMILES: FC(F)(F)Oc1cnccc1-n1cnnc1-c1nc2cc(C34CC(C3)C4)ccc2[nH]1] | 223 |
| 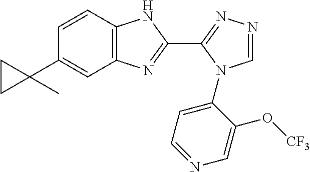 [SMILES: CC1(c2ccc3[nH]c(-c4nncn4-c4ccncc4OC(F)(F)F)nc3c2)CC1] | 224 |
| 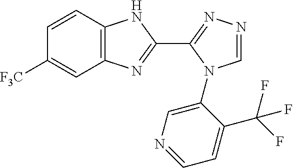 [SMILES: FC(F)(F)c1ccc2[nH]c(-c3nncn3-c3cnccc3C(F)(F)F)nc2c1] | 225 |
| 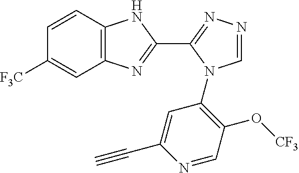 [SMILES: C#Cc1cc(-n2cnnc2-c2nc3cc(C(F)(F)F)ccc3[nH]2)c(OC(F)(F)F)cn1] | 226 |
| 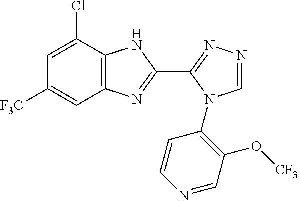 [SMILES: FC(F)(F)Oc1cnccc1-n1cnnc1-c1nc2cc(C(F)(F)F)cc(Cl)c2[nH]1] | 227 |
| 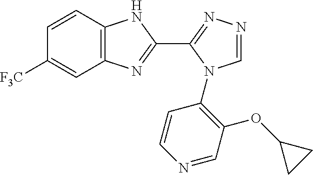 [SMILES: FC(F)(F)c1ccc2[nH]c(-c3nncn3-c3ccncc3OC3CC3)nc2c1] | 228 |
| 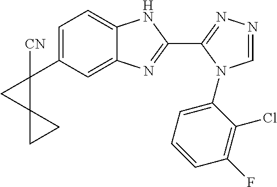 [SMILES: N#CC1(c2ccc3[nH]c(-c4nncn4-c4cccc(F)c4Cl)nc3c2)CC12CC2] | 229 |
| 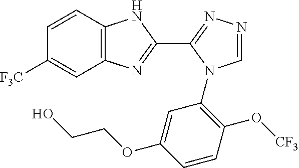 [SMILES: OCCOc1ccc(OC(F)(F)F)c(-n2cnnc2-c2nc3cc(C(F)(F)F)ccc3[nH]2)c1] | 230 |
| 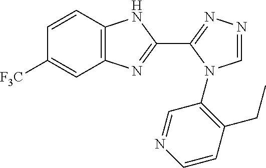 [SMILES: CCc1ccncc1-n1cnnc1-c1nc2cc(C(F)(F)F)ccc2[nH]1] | 231 |
| 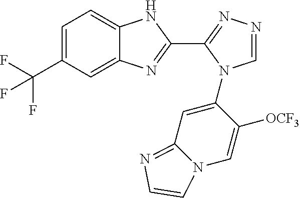 [SMILES: FC(F)(F)Oc1cn2ccnc2cc1-n1cnnc1-c1nc2cc(C(F)(F)F)ccc2[nH]1] | 232 |
| 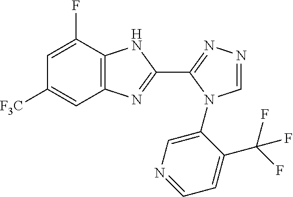 [SMILES: Fc1cc(C(F)(F)F)cc2nc(-c3nncn3-c3cnccc3C(F)(F)F)[nH]c12] | 233 |
| 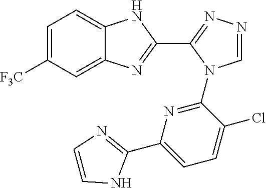 [SMILES: FC(F)(F)c1ccc2[nH]c(-c3nncn3-c3nc(-c4ncc[nH]4)ccc3Cl)nc2c1] | 234 |
| 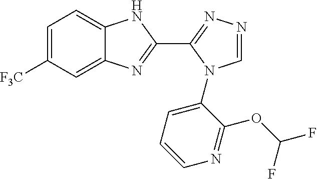 [SMILES: FC(F)Oc1ncccc1-n1cnnc1-c1nc2cc(C(F)(F)F)ccc2[nH]1] | 235 |
| 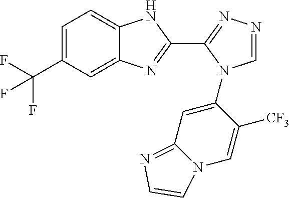 [SMILES: FC(F)(F)c1ccc2[nH]c(-c3nncn3-c3cc4nccn4cc3C(F)(F)F)nc2c1] | 236 |
| 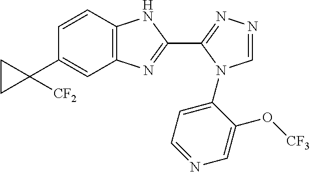 | 237 |
| 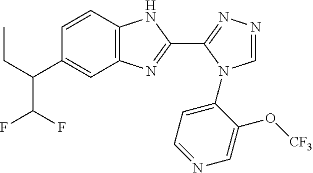 [SMILES: CCC(c1ccc2[nH]c(-c3nncn3-c3ccncc3OC(F)(F)F)nc2c1)C(F)F] | 238 |
| 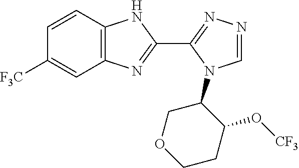 [SMILES: FC(F)(F)O[C@@H]1CCOC[C@H]1n1cnnc1-c1nc2cc(C(F)(F)F)ccc2[nH]1] | 239 |
| 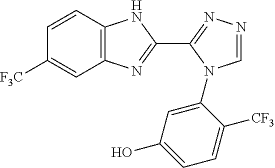 [SMILES: Oc1ccc(C(F)(F)F)c(-n2cnnc2-c2nc3cc(C(F)(F)F)ccc3[nH]2)c1] | 240 |
| 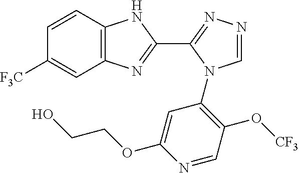 [SMILES: OCCOc1cc(-n2cnnc2-c2nc3cc(C(F)(F)F)ccc3[nH]2)c(OC(F)(F)F)cn1] | 241 |
| 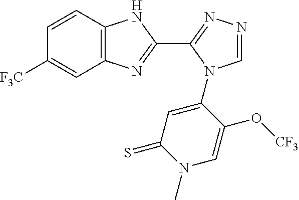 [SMILES: Cn1cc(OC(F)(F)F)c(-n2cnnc2-c2nc3cc(C(F)(F)F)ccc3[nH]2)cc1=S] | 242 |
| 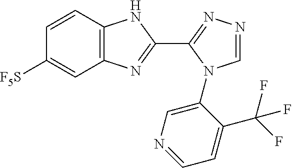 | 243 |
| 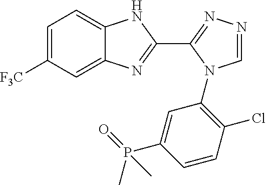 [SMILES: CP(C)(=O)c1ccc(Cl)c(-n2cnnc2-c2nc3cc(C(F)(F)F)ccc3[nH]2)c1] | 244 |
| 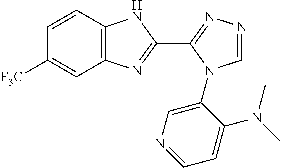 [SMILES: CN(C)c1ccncc1-n1cnnc1-c1nc2cc(C(F)(F)F)ccc2[nH]1] | 245 |
| 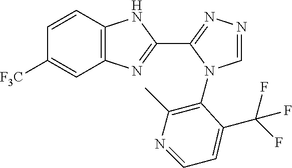 [SMILES: Cc1nccc(C(F)(F)F)c1-n1cnnc1-c1nc2cc(C(F)(F)F)ccc2[nH]1] | 246 |
| 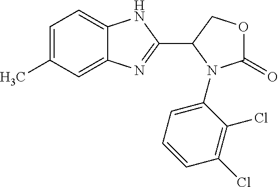 [SMILES: Cc1ccc2[nH]c(C3COC(=O)N3c3cccc(Cl)c3Cl)nc2c1] | 247 |
| 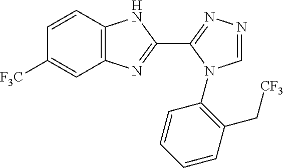 [SMILES: FC(F)(F)Cc1ccccc1-n1cnnc1-c1nc2cc(C(F)(F)F)ccc2[nH]1] | 248 |
| 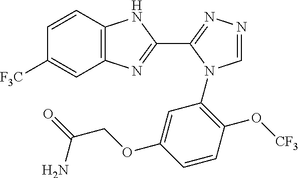 [SMILES: NC(=O)COc1ccc(OC(F)(F)F)c(-n2cnnc2-c2nc3cc(C(F)(F)F)ccc3[nH]2)c1] | 249 |
| 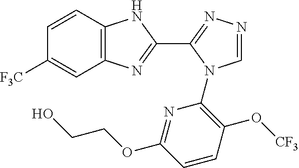 [SMILES: OCCOc1ccc(OC(F)(F)F)c(-n2cnnc2-c2nc3cc(C(F)(F)F)ccc3[nH]2)n1] | 250 |
| 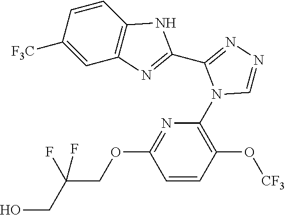 [SMILES: OCC(F)(F)COc1ccc(OC(F)(F)F)c(-n2cnnc2-c2nc3cc(C(F)(F)F)ccc3[nH]2)n1] | 251 |
| 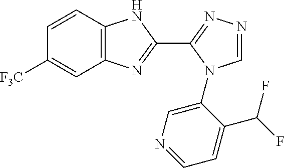 [SMILES: FC(F)c1ccncc1-n1cnnc1-c1nc2cc(C(F)(F)F)ccc2[nH]1] | 252 |
| 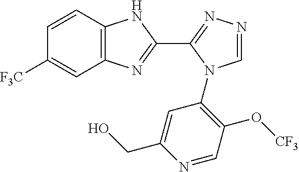 [SMILES: OCc1cc(-n2cnnc2-c2nc3cc(C(F)(F)F)ccc3[nH]2)c(OC(F)(F)F)cn1] | 253 |
| 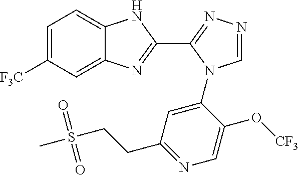 [SMILES: CS(=O)(=O)CCc1cc(-n2cnnc2-c2nc3cc(C(F)(F)F)ccc3[nH]2)c(OC(F)(F)F)cn1] | 254 |
| 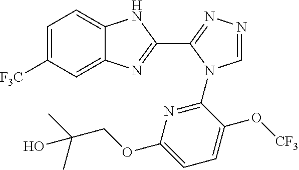 [SMILES: CC(C)(O)COc1ccc(OC(F)(F)F)c(-n2cnnc2-c2nc3cc(C(F)(F)F)ccc3[nH]2)n1] | 255 |
| 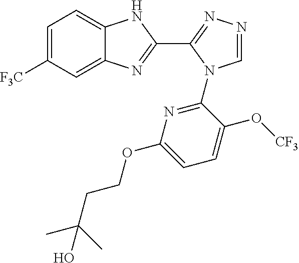 [SMILES: CC(C)(O)CCOc1ccc(OC(F)(F)F)c(-n2cnnc2-c2nc3cc(C(F)(F)F)ccc3[nH]2)n1] | 256 |
| 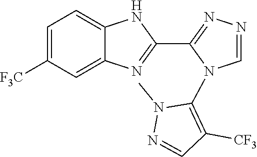 [SMILES: Cn1ncc(C(F)(F)F)c1-n1cnnc1-c1nc2cc(C(F)(F)F)ccc2[nH]1] | 257 |
| 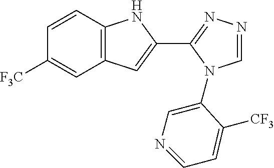 [SMILES: FC(F)(F)c1ccc2[nH]c(-c3nncn3-c3cnccc3C(F)(F)F)cc2c1] | 258 |
| 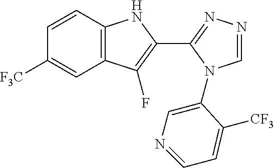 [SMILES: Fc1c(-c2nncn2-c2cnccc2C(F)(F)F)[nH]c2ccc(C(F)(F)F)cc12] | 259 |
| 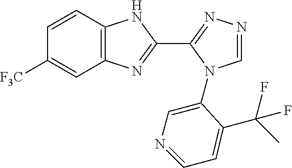 [SMILES: CC(F)(F)c1ccncc1-n1cnnc1-c1nc2cc(C(F)(F)F)ccc2[nH]1] | 260 |
| 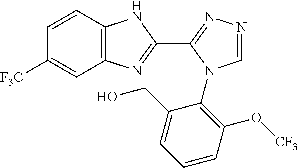 [SMILES: OCc1cccc(OC(F)(F)F)c1-n1cnnc1-c1nc2cc(C(F)(F)F)ccc2[nH]1] | 261 |
| 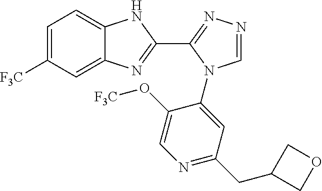 [SMILES: FC(F)(F)Oc1cnc(CC2COC2)cc1-n1cnnc1-c1nc2cc(C(F)(F)F)ccc2[nH]1] | 262 |
| 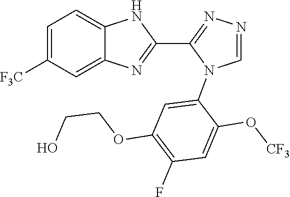 [SMILES: OCCOc1cc(-n2cnnc2-c2nc3cc(C(F)(F)F)ccc3[nH]2)c(OC(F)(F)F)cc1F] | 263 |
| 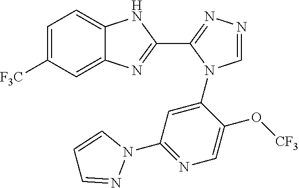 [SMILES: FC(F)(F)Oc1cnc(-n2cccn2)cc1-n1cnnc1-c1nc2cc(C(F)(F)F)ccc2[nH]1] | 264 |
| 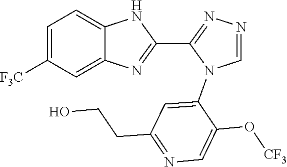 [SMILES: OCCc1cc(-n2cnnc2-c2nc3cc(C(F)(F)F)ccc3[nH]2)c(OC(F)(F)F)cn1] | 265 |
| 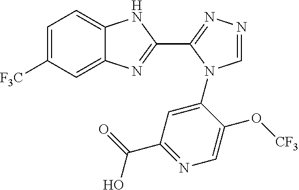 [SMILES: O=C(O)c1cc(-n2cnnc2-c2nc3cc(C(F)(F)F)ccc3[nH]2)c(OC(F)(F)F)cn1] | 266 |
| 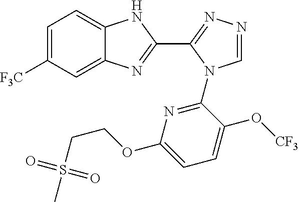 [SMILES: CS(=O)(=O)CCOc1ccc(OC(F)(F)F)c(-n2cnnc2-c2nc3cc(C(F)(F)F)ccc3[nH]2)n1] | 267 |
| 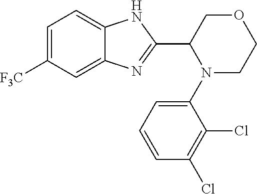 [SMILES: FC(F)(F)c1ccc2[nH]c(C3COCCN3c3cccc(Cl)c3Cl)nc2c1] | 268 |
| 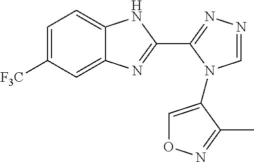 [SMILES: Cc1nocc1-n1cnnc1-c1nc2cc(C(F)(F)F)ccc2[nH]1] | 269 |
| 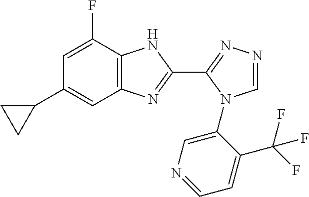 [SMILES: Fc1cc(C2CC2)cc2nc(-c3nncn3-c3cnccc3C(F)(F)F)[nH]c12] | 270 |
| 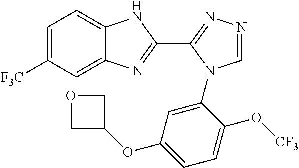 [SMILES: FC(F)(F)Oc1ccc(OC2COC2)cc1-n1cnnc1-c1nc2cc(C(F)(F)F)ccc2[nH]1] | 271 |
| 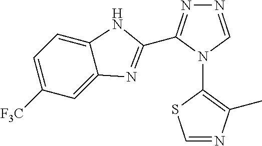 [SMILES: Cc1ncsc1-n1cnnc1-c1nc2cc(C(F)(F)F)ccc2[nH]1] | 272 |
| 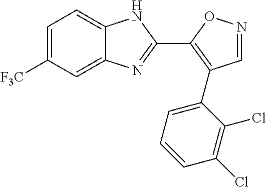 [SMILES: FC(F)(F)c1ccc2[nH]c(-c3oncc3-c3cccc(Cl)c3Cl)nc2c1] | 273 |
| 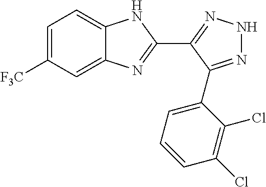 [SMILES: FC(F)(F)c1ccc2[nH]c(-c3n[nH]nc3-c3cccc(Cl)c3Cl)nc2c1] | 274 |
| 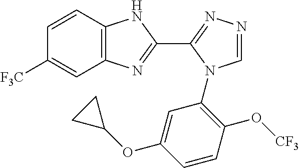 [SMILES: FC(F)(F)Oc1ccc(OC2CC2)cc1-n1cnnc1-c1nc2cc(C(F)(F)F)ccc2[nH]1] | 275 |
| 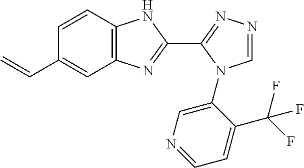 [SMILES: C=Cc1ccc2[nH]c(-c3nncn3-c3cnccc3C(F)(F)F)nc2c1] | 276 |
| 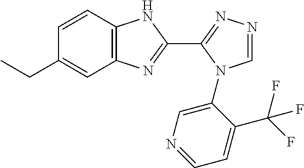 [SMILES: CCc1ccc2[nH]c(-c3nncn3-c3cnccc3C(F)(F)F)nc2c1] | 277 |
| 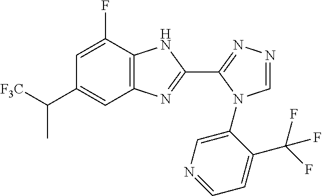 [SMILES: CC(c1cc(F)c2[nH]c(-c3nncn3-c3cnccc3C(F)(F)F)nc2c1)C(F)(F)F] | 278 |
| 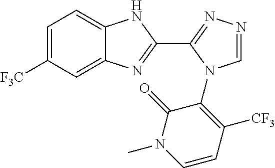 [SMILES: Cn1ccc(C(F)(F)F)c(-n2cnnc2-c2nc3cc(C(F)(F)F)ccc3[nH]2)c1=O] | 279 |
| 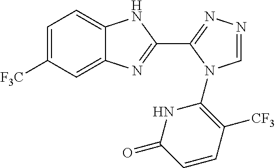 [SMILES: O=c1ccc(C(F)(F)F)c(-n2cnnc2-c2nc3cc(C(F)(F)F)ccc3[nH]2)[nH]1] | 280 |
| 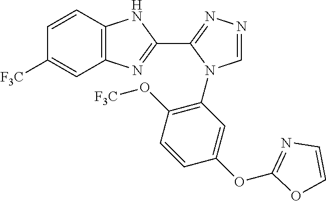 [SMILES: FC(F)(F)Oc1ccc(Oc2ncco2)cc1-n1cnnc1-c1nc2cc(C(F)(F)F)ccc2[nH]1] | 281 |
| 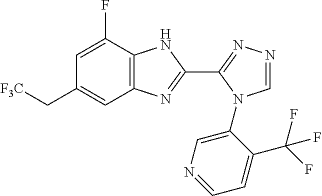 [SMILES: Fc1cc(CC(F)(F)F)cc2nc(-c3nncn3-c3cnccc3C(F)(F)F)[nH]c12] | 282 |
| 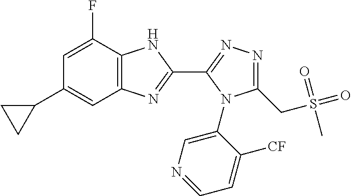 | 283 |
| 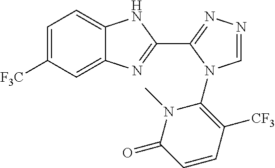 [SMILES: Cn1c(-n2cnnc2-c2nc3cc(C(F)(F)F)ccc3[nH]2)c(C(F)(F)F)ccc1=O] | 284 |
| 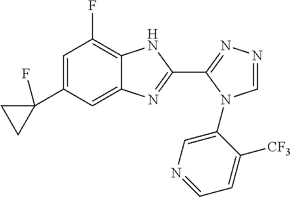 [SMILES: Fc1cc(C2(F)CC2)cc2nc(-c3nncn3-c3cnccc3C(F)(F)F)[nH]c12] | 285 |
| 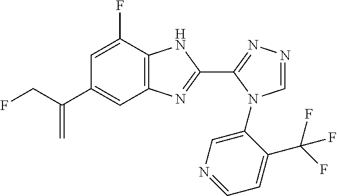 [SMILES: C=C(CF)c1cc(F)c2[nH]c(-c3nncn3-c3cnccc3C(F)(F)F)nc2c1] | 286 |
| 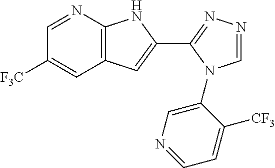 [SMILES: FC(F)(F)c1cnc2[nH]c(-c3nncn3-c3cnccc3C(F)(F)F)cc2c1] | 287 |
| 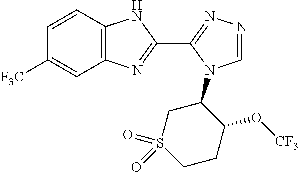 [SMILES: O=S1(=O)CC[C@@H](OC(F)(F)F)[C@H](n2cnnc2-c2nc3cc(C(F)(F)F)ccc3[nH]2)C1] | 288 |
| 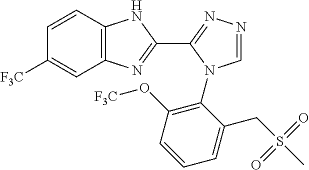 [SMILES: CS(=O)(=O)Cc1cccc(OC(F)(F)F)c1-n1cnnc1-c1nc2cc(C(F)(F)F)ccc2[nH]1] | 289 |
| 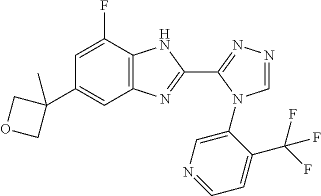 [SMILES: CC1(c2cc(F)c3[nH]c(-c4nncn4-c4cnccc4C(F)(F)F)nc3c2)COC1] | 290 |
| 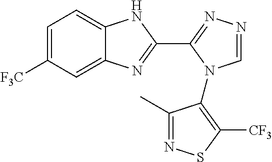 [SMILES: Cc1nsc(C(F)(F)F)c1-n1cnnc1-c1nc2cc(C(F)(F)F)ccc2[nH]1] | 291 |
| 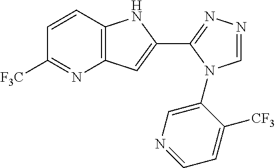 [SMILES: FC(F)(F)c1ccc2[nH]c(-c3nncn3-c3cnccc3C(F)(F)F)cc2n1] | 292 |
| 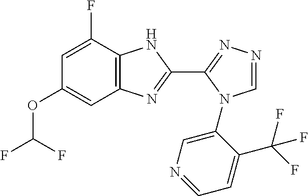 [SMILES: Fc1cc(OC(F)F)cc2nc(-c3nncn3-c3cnccc3C(F)(F)F)[nH]c12] | 293 |
| 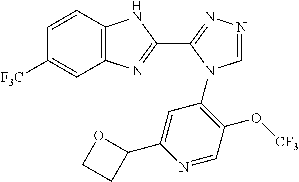 [SMILES: FC(F)(F)Oc1cnc(C2CCO2)cc1-n1cnnc1-c1nc2cc(C(F)(F)F)ccc2[nH]1] | 294 |
| 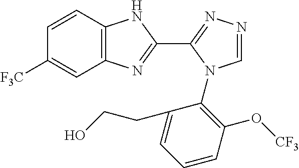 [SMILES: OCCc1cccc(OC(F)(F)F)c1-n1cnnc1-c1nc2cc(C(F)(F)F)ccc2[nH]1] | 295 |
| 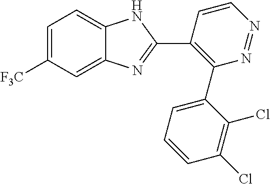 [SMILES: FC(F)(F)c1ccc2[nH]c(-c3ccnnc3-c3cccc(Cl)c3Cl)nc2c1] | 296 |
| 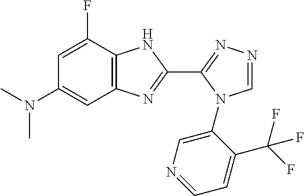 [SMILES: CN(C)c1cc(F)c2[nH]c(-c3nncn3-c3cnccc3C(F)(F)F)nc2c1] | 297 |
| 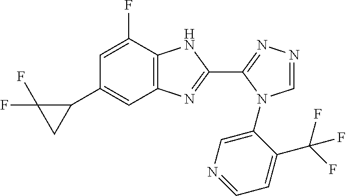 [SMILES: Fc1cc(C2CC2(F)F)cc2nc(-c3nncn3-c3cnccc3C(F)(F)F)[nH]c12] | 298 |
| 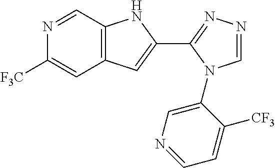 [SMILES: FC(F)(F)c1cc2cc(-c3nncn3-c3cnccc3C(F)(F)F)[nH]c2cn1] | 299 |
| 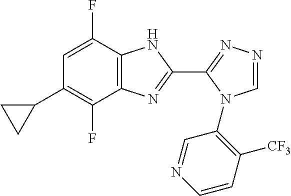 [SMILES: Fc1c(C2CC2)cc(F)c2[nH]c(-c3nncn3-c3cnccc3C(F)(F)F)nc12] | 300 |
| 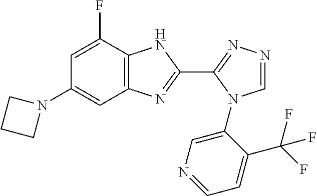 [SMILES: Fc1cc(N2CCC2)cc2nc(-c3nncn3-c3cnccc3C(F)(F)F)[nH]c12] | 301 |
| 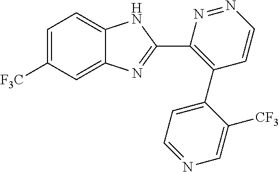 [SMILES: FC(F)(F)c1ccc2[nH]c(-c3nnccc3-c3ccncc3C(F)(F)F)nc2c1] | 302 |
| 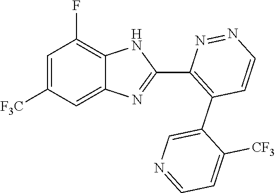 [SMILES: Fc1cc(C(F)(F)F)cc2nc(-c3nnccc3-c3cnccc3C(F)(F)F)[nH]c12] | 303 |
| 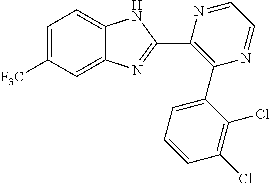 [SMILES: FC(F)(F)c1ccc2[nH]c(-c3nccnc3-c3cccc(Cl)c3Cl)nc2c1] | 304 |
| 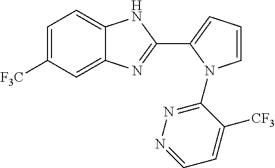 [SMILES: FC(F)(F)c1ccc2[nH]c(-c3cccn3-c3nnccc3C(F)(F)F)nc2c1] | 305 |
| 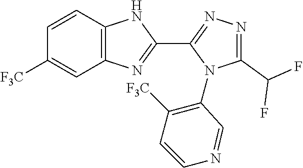 [SMILES: FC(F)c1nnc(-c2nc3cc(C(F)(F)F)ccc3[nH]2)n1-c1cnccc1C(F)(F)F] | 306 |
| 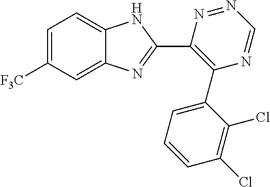 [SMILES: FC(F)(F)c1ccc2[nH]c(-c3nncnc3-c3cccc(Cl)c3Cl)nc2c1] | 307 |
| 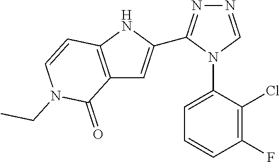 [SMILES: CCn1ccc2[nH]c(-c3nncn3-c3cccc(F)c3Cl)cc2c1=O] | 308 |
| 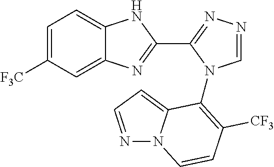 [SMILES: FC(F)(F)c1ccc2[nH]c(-c3nncn3-c3c(C(F)(F)F)ccn4nccc34)nc2c1] | 309 |
| 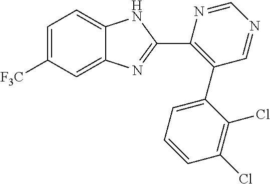 [SMILES: FC(F)(F)c1ccc2[nH]c(-c3ncncc3-c3cccc(Cl)c3Cl)nc2c1] | 310 |
| 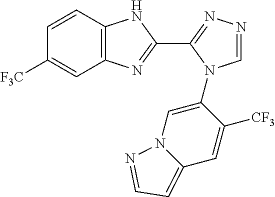 [SMILES: FC(F)(F)c1ccc2[nH]c(-c3nncn3-c3cn4nccc4cc3C(F)(F)F)nc2c1] | 311 |
| 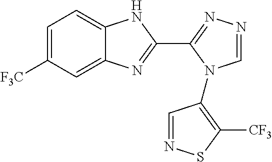 [SMILES: FC(F)(F)c1ccc2[nH]c(-c3nncn3-c3cnsc3C(F)(F)F)nc2c1] | 312 |
| 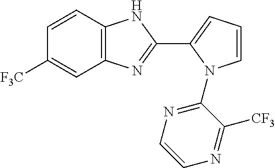 [SMILES: FC(F)(F)c1ccc2[nH]c(-c3cccn3-c3nccnc3C(F)(F)F)nc2c1] | 313 |
| 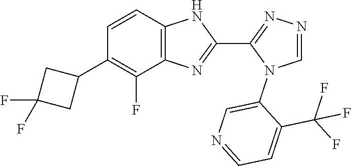 [SMILES: Fc1c(C2CC(F)(F)C2)ccc2[nH]c(-c3nncn3-c3cnccc3C(F)(F)F)nc12] | 314 |
| 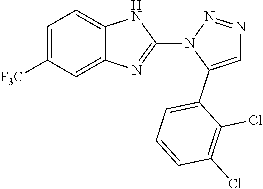 [SMILES: FC(F)(F)c1ccc2[nH]c(-n3nncc3-c3cccc(Cl)c3Cl)nc2c1] | 315 |
| 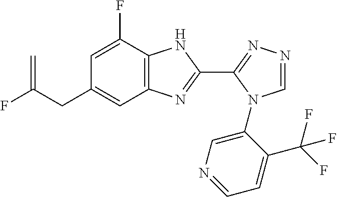 [SMILES: C=C(F)Cc1cc(F)c2[nH]c(-c3nncn3-c3cnccc3C(F)(F)F)nc2c1] | 316 |
| 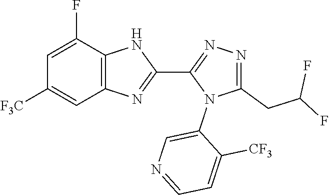 [SMILES: Fc1cc(C(F)(F)F)cc2nc(-c3nnc(CC(F)F)n3-c3cnccc3C(F)(F)F)[nH]c12] | 317 |
| 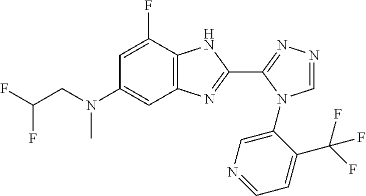 [SMILES: CN(CC(F)F)c1cc(F)c2[nH]c(-c3nncn3-c3cnccc3C(F)(F)F)nc2c1] | 318 |
| 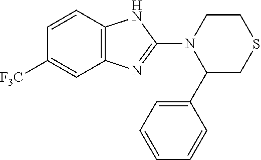 [SMILES: FC(F)(F)c1ccc2[nH]c(N3CCSCC3c3ccccc3)nc2c1] | 319 |
| 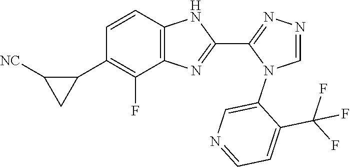 [SMILES: N#CC1CC1c1ccc2[nH]c(-c3nncn3-c3cnccc3C(F)(F)F)nc2c1F] | 320 |
| 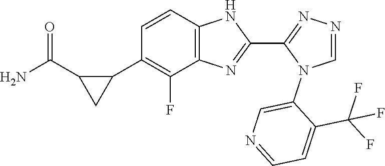 [SMILES: NC(=O)C1CC1c1ccc2[nH]c(-c3nncn3-c3cnccc3C(F)(F)F)nc2c1F] | 321 |
| 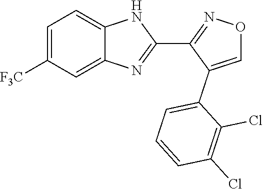 [SMILES: FC(F)(F)c1ccc2[nH]c(-c3nocc3-c3cccc(Cl)c3Cl)nc2c1] | 322 |
| 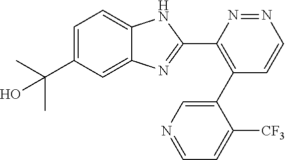 [SMILES: CC(C)(O)c1ccc2[nH]c(-c3nnccc3-c3cnccc3C(F)(F)F)nc2c1] | 323 |
| 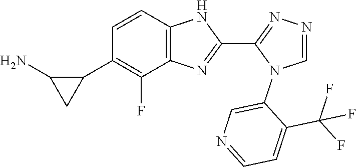 [SMILES: NC1CC1c1ccc2[nH]c(-c3nncn3-c3cnccc3C(F)(F)F)nc2c1F] | 324 |
| 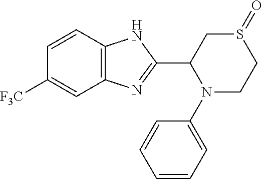 [SMILES: O=S1CCN(c2ccccc2)C(c2nc3cc(C(F)(F)F)ccc3[nH]2)C1] | 325 |
| 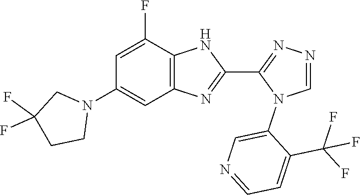 [SMILES: Fc1cc(N2CCC(F)(F)C2)cc2nc(-c3nncn3-c3cnccc3C(F)(F)F)[nH]c12] | 326 |
| 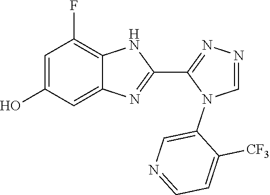 [SMILES: Oc1cc(F)c2[nH]c(-c3nncn3-c3cnccc3C(F)(F)F)nc2c1] | 327 |
| 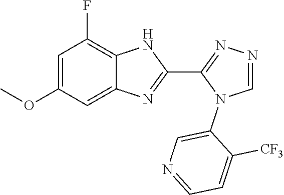 [SMILES: COc1cc(F)c2[nH]c(-c3nncn3-c3cnccc3C(F)(F)F)nc2c1] | 328 |
| 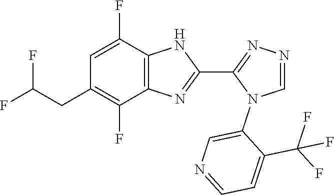 [SMILES: Fc1c(CC(F)F)cc(F)c2[nH]c(-c3nncn3-c3cnccc3C(F)(F)F)nc12] | 329 |
| 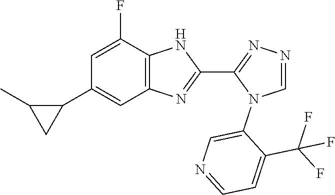 [SMILES: CC1CC1c1cc(F)c2[nH]c(-c3nncn3-c3cnccc3C(F)(F)F)nc2c1] | 330 |
| 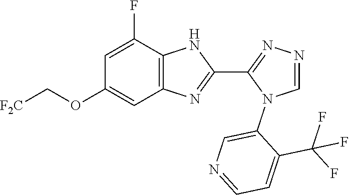 | 331 |
| 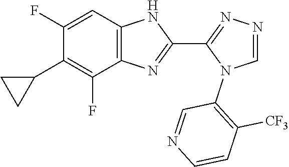 [SMILES: Fc1cc2[nH]c(-c3nncn3-c3cnccc3C(F)(F)F)nc2c(F)c1C1CC1] | 332 |
| 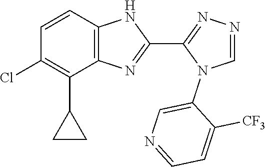 [SMILES: FC(F)(F)c1ccncc1-n1cnnc1-c1nc2c(C3CC3)c(Cl)ccc2[nH]1] | 333 |
| 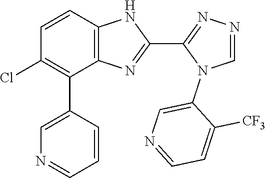 [SMILES: FC(F)(F)c1ccncc1-n1cnnc1-c1nc2c(-c3cccnc3)c(Cl)ccc2[nH]1] | 334 |
| 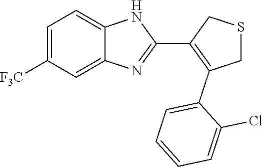 [SMILES: FC(F)(F)c1ccc2[nH]c(C3=C(c4ccccc4Cl)CSC3)nc2c1] | 335 |
| 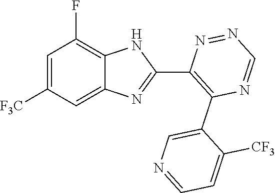 [SMILES: Fc1cc(C(F)(F)F)cc2nc(-c3nncnc3-c3cnccc3C(F)(F)F)[nH]c12] | 336 |
| 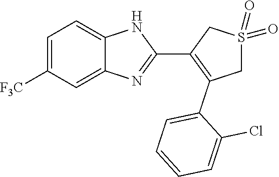 [SMILES: O=S1(=O)CC(c2nc3cc(C(F)(F)F)ccc3[nH]2)=C(c2ccccc2Cl)C1] | 337 |
| 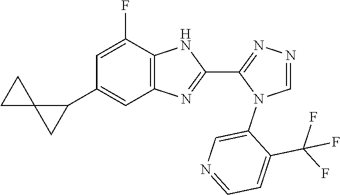 [SMILES: Fc1cc(C2CC23CC3)cc2nc(-c3nncn3-c3cnccc3C(F)(F)F)[nH]c12] | 338 |
| 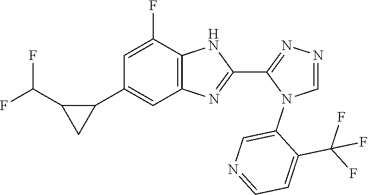 [SMILES: Fc1cc(C2CC2C(F)F)cc2nc(-c3nncn3-c3cnccc3C(F)(F)F)[nH]c12] | 339 |
| 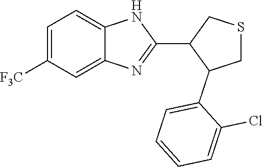 [SMILES: FC(F)(F)c1ccc2[nH]c(C3CSCC3c3ccccc3Cl)nc2c1] | 340 |
| 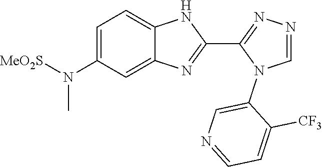 [SMILES: CN(c1ccc2[nH]c(-c3nncn3-c3cnccc3C(F)(F)F)nc2c1)S(C)(=O)=O] | 341 |
| 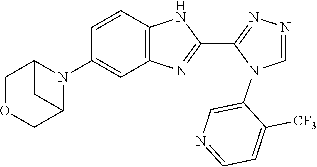 [SMILES: FC(F)(F)c1ccncc1-n1cnnc1-c1nc2cc(N3C4COCC3C4)ccc2[nH]1] | 342 |
| 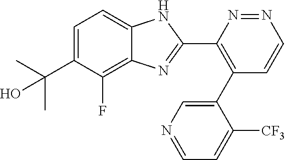 [SMILES: CC(C)(O)c1ccc2[nH]c(-c3nnccc3-c3cnccc3C(F)(F)F)nc2c1F] | 343 |
| 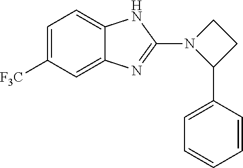 [SMILES: FC(F)(F)c1ccc2[nH]c(N3CCC3c3ccccc3)nc2c1] | 344 |
| 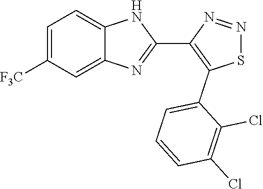 [SMILES: FC(F)(F)c1ccc2[nH]c(-c3nnsc3-c3cccc(Cl)c3Cl)nc2c1] | 345 |
| 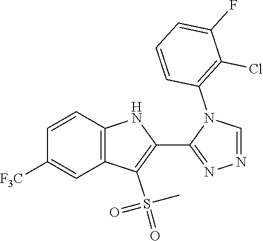 [SMILES: CS(=O)(=O)c1c(-c2nncn2-c2cccc(F)c2Cl)[nH]c2ccc(C(F)(F)F)cc12] | 346 |
| 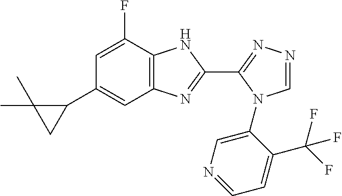 [SMILES: CC1(C)CC1c1cc(F)c2[nH]c(-c3nncn3-c3cnccc3C(F)(F)F)nc2c1] | 347 |
| 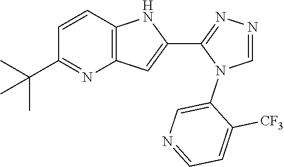 [SMILES: CC(C)(C)c1ccc2[nH]c(-c3nncn3-c3cnccc3C(F)(F)F)cc2n1] | 348 |
| 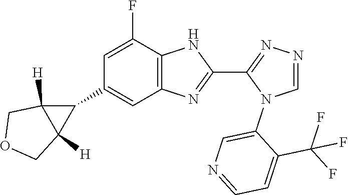 [SMILES: Fc1cc([C@@H]2[C@@H]3COC[C@@H]32)cc2nc(-c3nncn3-c3cnccc3C(F)(F)F)[nH]c12] | 349 |
| 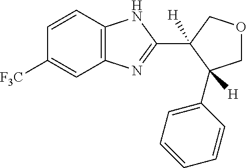 [SMILES: FC(F)(F)c1ccc2[nH]c([C@H]3COC[C@@H]3c3ccccc3)nc2c1] | 350 |
| 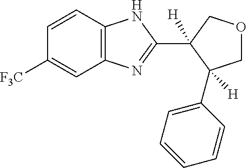 [SMILES: FC(F)(F)c1ccc2[nH]c([C@H]3COC[C@H]3c3ccccc3)nc2c1] | 351 |
| 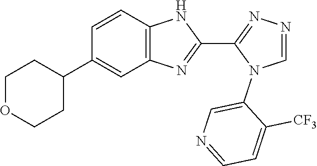 [SMILES: FC(F)(F)c1ccncc1-n1cnnc1-c1nc2cc(C3CCOCC3)ccc2[nH]1] | 352 |
| 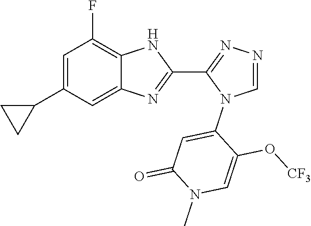 [SMILES: Cn1cc(OC(F)(F)F)c(-n2cnnc2-c2nc3cc(C4CC4)cc(F)c3[nH]2)cc1=O] | 353 |
| 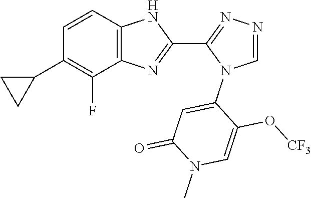 [SMILES: Cn1cc(OC(F)(F)F)c(-n2cnnc2-c2nc3c(F)c(C4CC4)ccc3[nH]2)cc1=O] | 354 |
| 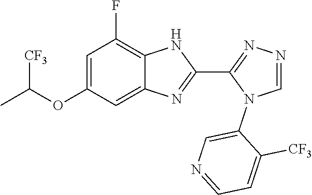 [SMILES: CC(Oc1cc(F)c2[nH]c(-c3nncn3-c3cnccc3C(F)(F)F)nc2c1)C(F)(F)F] | 355 |
| 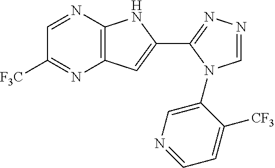 [SMILES: FC(F)(F)c1cnc2[nH]c(-c3nncn3-c3cnccc3C(F)(F)F)cc2n1] | 356 |
| 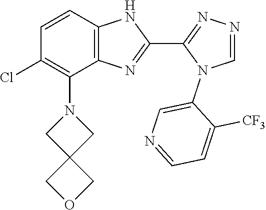 [SMILES: FC(F)(F)c1ccncc1-n1cnnc1-c1nc2c(N3CC4(COC4)C3)c(Cl)ccc2[nH]1] | 357 |
| 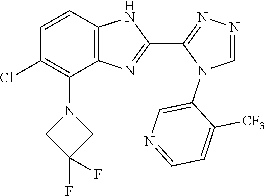 [SMILES: FC1(F)CN(c2c(Cl)ccc3[nH]c(-c4nncn4-c4cnccc4C(F)(F)F)nc23)C1] | 358 |
| 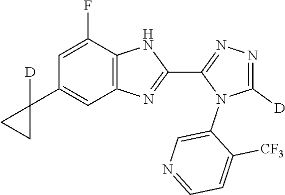 [SMILES: [2H]c1nnc(-c2nc3cc(C4([2H])CC4)cc(F)c3[nH]2)n1-c1cnccc1C(F)(F)F] | 359 |
| 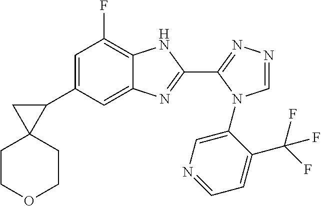 [SMILES: Fc1cc(C2CC23CCOCC3)cc2nc(-c3nncn3-c3cnccc3C(F)(F)F)[nH]c12] | 360 |
| 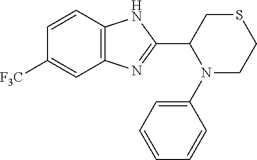 [SMILES: FC(F)(F)c1ccc2[nH]c(C3CSCCN3c3ccccc3)nc2c1] | 361 |
| 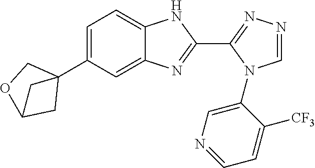 [SMILES: FC(F)(F)c1ccncc1-n1cnnc1-c1nc2cc(C34COC(C3)C4)ccc2[nH]1] | 362 |
| 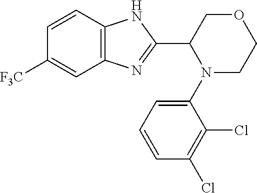 [SMILES: FC(F)(F)c1ccc2[nH]c(C3COCCN3c3cccc(Cl)c3Cl)nc2c1] | 363 |
| 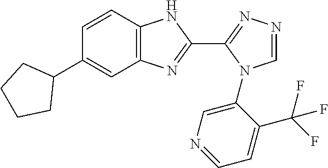 [SMILES: FC(F)(F)c1ccncc1-n1cnnc1-c1nc2cc(C3CCCC3)ccc2[nH]1] | 364 |
| 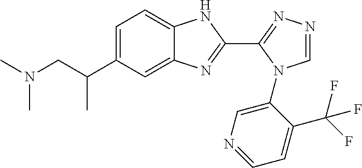 [SMILES: CC(CN(C)C)c1ccc2[nH]c(-c3nncn3-c3cnccc3C(F)(F)F)nc2c1] | 365 |
| 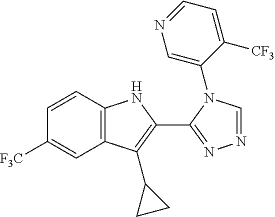 [SMILES: FC(F)(F)c1ccc2[nH]c(-c3nncn3-c3cnccc3C(F)(F)F)c(C3CC3)c2c1] | 366 |
| 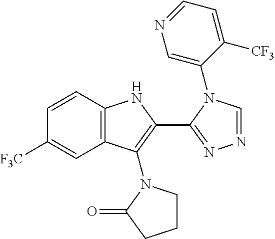 [SMILES: O=C1CCCN1c1c(-c2nncn2-c2cnccc2C(F)(F)F)[nH]c2ccc(C(F)(F)F)cc12] | 367 |
| 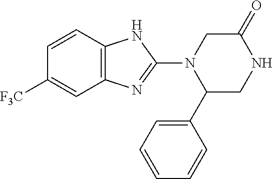 [SMILES: O=C1CN(c2nc3cc(C(F)(F)F)ccc3[nH]2)C(c2ccccc2)CN1] | 368 |
| 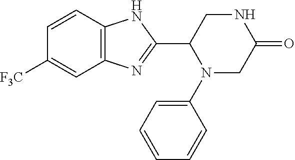 [SMILES: O=C1CN(c2ccccc2)C(c2nc3cc(C(F)(F)F)ccc3[nH]2)CN1] | 369 |
| 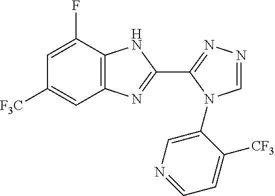 [SMILES: Fc1cc(C(F)(F)F)cc2nc(-c3nncn3-c3cnccc3C(F)(F)F)[nH]c12] | 370 |
| 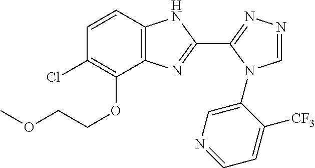 [SMILES: COCCOc1c(Cl)ccc2[nH]c(-c3nncn3-c3cnccc3C(F)(F)F)nc12] | 371 |
| 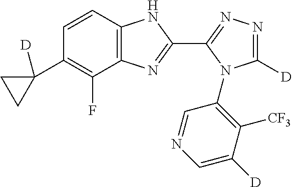 [SMILES: [2H]c1cncc(-n2c([2H])nnc2-c2nc3c(F)c(C4([2H])CC4)ccc3[nH]2)c1C(F)(F)F] | 372 |
| 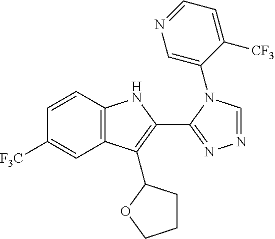 [SMILES: FC(F)(F)c1ccc2[nH]c(-c3nncn3-c3cnccc3C(F)(F)F)c(C3CCCO3)c2c1] | 373 |
| 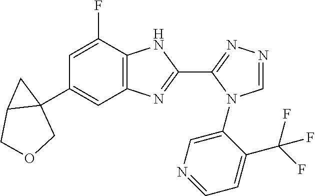 [SMILES: Fc1cc(C23COCC2C3)cc2nc(-c3nncn3-c3cnccc3C(F)(F)F)[nH]c12] | 374 |
| 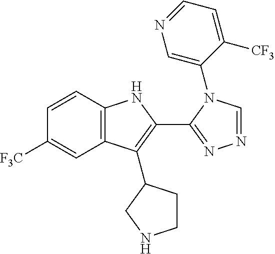 [SMILES: FC(F)(F)c1ccc2[nH]c(-c3nncn3-c3cnccc3C(F)(F)F)c(C3CCNC3)c2c1] | 375 |
| 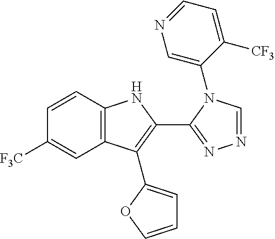 [SMILES: FC(F)(F)c1ccc2[nH]c(-c3nncn3-c3cnccc3C(F)(F)F)c(-c3ccco3)c2c1] | 376 |
| 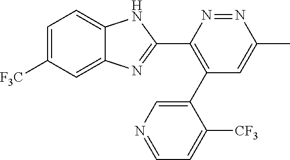 [SMILES: Cc1cc(-c2cnccc2C(F)(F)F)c(-c2nc3cc(C(F)(F)F)ccc3[nH]2)nn1] | 377 |
| 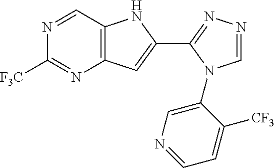 [SMILES: FC(F)(F)c1ncc2[nH]c(-c3nncn3-c3cnccc3C(F)(F)F)cc2n1] | 378 |
| 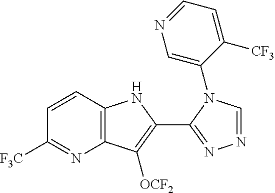 | 379 |
| 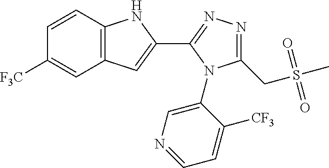 [SMILES: CS(=O)(=O)Cc1nnc(-c2cc3cc(C(F)(F)F)ccc3[nH]2)n1-c1cnccc1C(F)(F)F] | 380 |
| 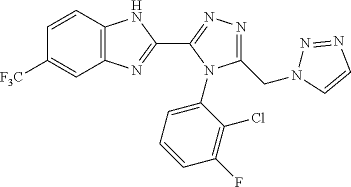 [SMILES: Fc1cccc(-n2c(Cn3ccnn3)nnc2-c2nc3cc(C(F)(F)F)ccc3[nH]2)c1Cl] | 381 |
| 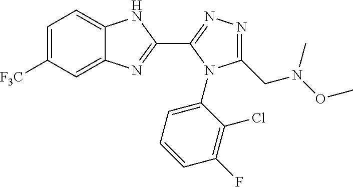 [SMILES: CON(C)Cc1nnc(-c2nc3cc(C(F)(F)F)ccc3[nH]2)n1-c1cccc(F)c1Cl] | 382 |
| 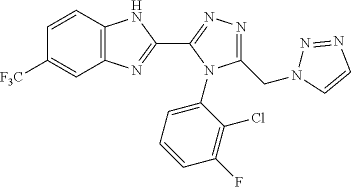 [SMILES: Fc1cccc(-n2c(Cn3ccnn3)nnc2-c2nc3cc(C(F)(F)F)ccc3[nH]2)c1Cl] | 383 |
| 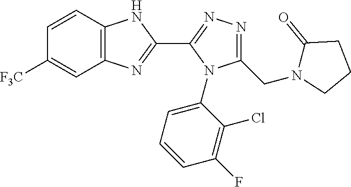 [SMILES: O=C1CCCN1Cc1nnc(-c2nc3cc(C(F)(F)F)ccc3[nH]2)n1-c1cccc(F)c1Cl] | 384 |
| 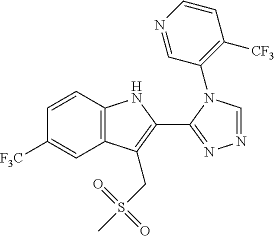 [SMILES: CS(=O)(=O)Cc1c(-c2nncn2-c2cnccc2C(F)(F)F)[nH]c2ccc(C(F)(F)F)cc12] | 385 |
| 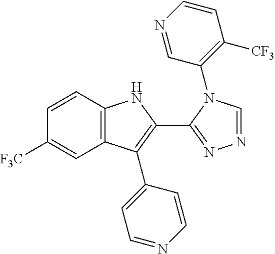 [SMILES: FC(F)(F)c1ccc2[nH]c(-c3nncn3-c3cnccc3C(F)(F)F)c(-c3ccncc3)c2c1] | 386 |
| 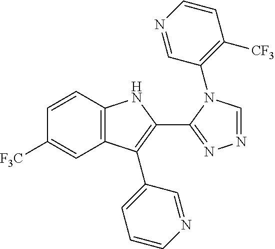 [SMILES: FC(F)(F)c1ccc2[nH]c(-c3nncn3-c3cnccc3C(F)(F)F)c(-c3cccnc3)c2c1] | 387 |
| 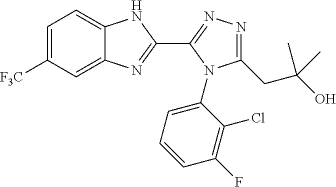 [SMILES: CC(C)(O)Cc1nnc(-c2nc3cc(C(F)(F)F)ccc3[nH]2)n1-c1cccc(F)c1Cl] | 388 |
| 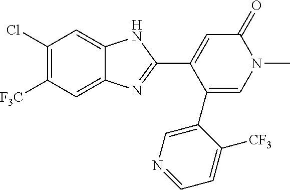 [SMILES: Cn1cc(-c2cnccc2C(F)(F)F)c(-c2nc3cc(C(F)(F)F)c(Cl)cc3[nH]2)cc1=O] | 389 |
| 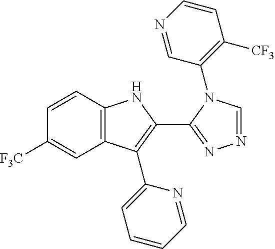 [SMILES: FC(F)(F)c1ccc2[nH]c(-c3nncn3-c3cnccc3C(F)(F)F)c(-c3ccccn3)c2c1] | 390 |
| 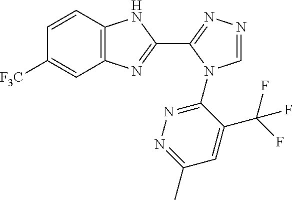 [SMILES: Cc1cc(C(F)(F)F)c(-n2cnnc2-c2nc3cc(C(F)(F)F)ccc3[nH]2)nn1] | 391 |
| 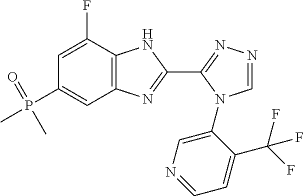 [SMILES: CP(C)(=O)c1cc(F)c2[nH]c(-c3nncn3-c3cnccc3C(F)(F)F)nc2c1] | 392 |
| 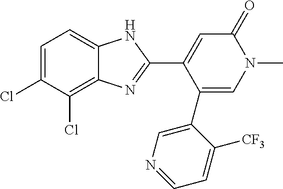 [SMILES: Cn1cc(-c2cnccc2C(F)(F)F)c(-c2nc3c(Cl)c(Cl)ccc3[nH]2)cc1=O] | 393 |
| 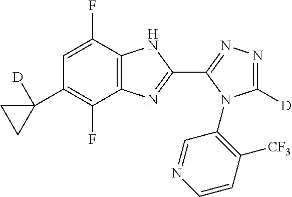 [SMILES: [2H]c1nnc(-c2nc3c(F)c(C4([2H])CC4)cc(F)c3[nH]2)n1-c1cnccc1C(F)(F)F] | 394 |
| 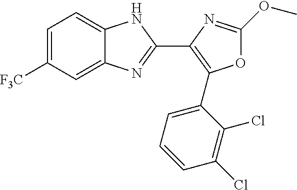 [SMILES: COc1nc(-c2nc3cc(C(F)(F)F)ccc3[nH]2)c(-c2cccc(Cl)c2Cl)o1] | 395 |
| 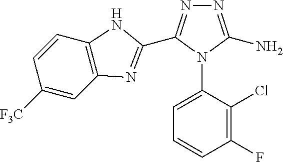 [SMILES: Nc1nnc(-c2nc3cc(C(F)(F)F)ccc3[nH]2)n1-c1cccc(F)c1Cl] | 396 |
| 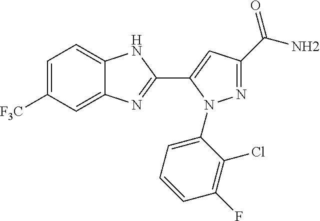 [SMILES: NC(=O)c1cc(-c2nc3cc(C(F)(F)F)ccc3[nH]2)n(-c2cccc(F)c2Cl)n1] | 397 |
| 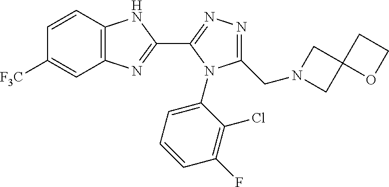 [SMILES: Fc1cccc(-n2c(CN3CC4(CCO4)C3)nnc2-c2nc3cc(C(F)(F)F)ccc3[nH]2)c1Cl] | 398 |
| 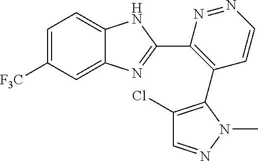 [SMILES: Cn1ncc(Cl)c1-c1ccnnc1-c1nc2cc(C(F)(F)F)ccc2[nH]1] | 399 |
| 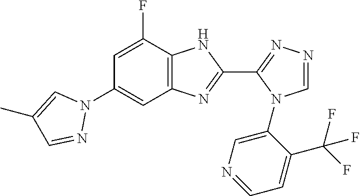 [SMILES: Cc1cnn(-c2cc(F)c3[nH]c(-c4nncn4-c4cnccc4C(F)(F)F)nc3c2)c1] | 400 |
| 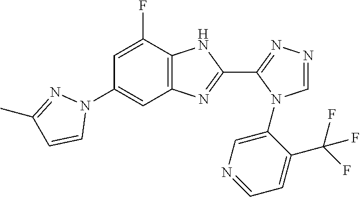 [SMILES: Cc1ccn(-c2cc(F)c3[nH]c(-c4nncn4-c4cnccc4C(F)(F)F)nc3c2)n1] | 401 |
| 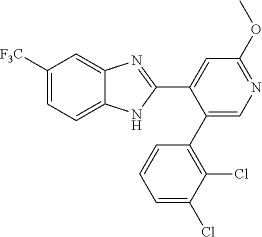 [SMILES: COc1cc(-c2nc3cc(C(F)(F)F)ccc3[nH]2)c(-c2cccc(Cl)c2Cl)cn1] | 402 |
| 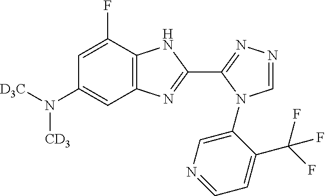 [SMILES: [2H]C([2H])([2H])N(c1cc(F)c2[nH]c(-c3nncn3-c3cnccc3C(F)(F)F)nc2c1)C([2H])([2H])[2H]] | 403 |
| 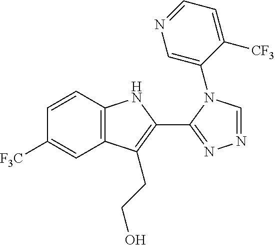 [SMILES: OCCc1c(-c2nncn2-c2cnccc2C(F)(F)F)[nH]c2ccc(C(F)(F)F)cc12] | 404 |
| 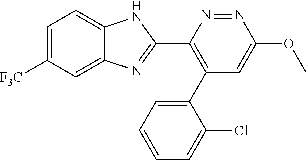 [SMILES: COc1cc(-c2ccccc2Cl)c(-c2nc3cc(C(F)(F)F)ccc3[nH]2)nn1] | 405 |
| 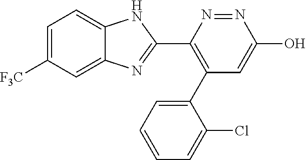 [SMILES: Oc1cc(-c2ccccc2Cl)c(-c2nc3cc(C(F)(F)F)ccc3[nH]2)nn1] | 406 |
| 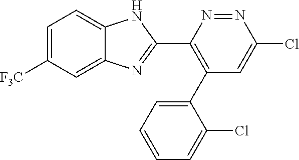 [SMILES: FC(F)(F)c1ccc2[nH]c(-c3nnc(Cl)cc3-c3ccccc3Cl)nc2c1] | 407 |
| 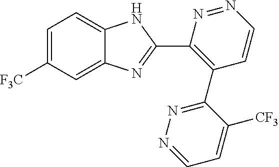 [SMILES: FC(F)(F)c1ccc2[nH]c(-c3nnccc3-c3nnccc3C(F)(F)F)nc2c1] | 408 |
| 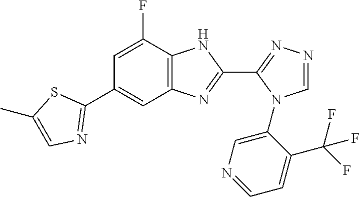 [SMILES: Cc1cnc(-c2cc(F)c3[nH]c(-c4nncn4-c4cnccc4C(F)(F)F)nc3c2)s1] | 409 |
| 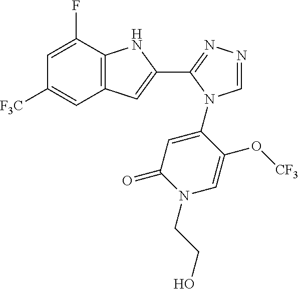 [SMILES: O=c1cc(-n2cnnc2-c2cc3cc(C(F)(F)F)cc(F)c3[nH]2)c(OC(F)(F)F)cn1CCO] | 410 |
| 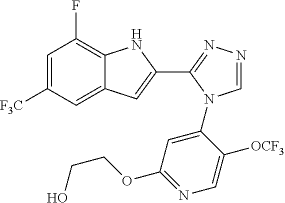 [SMILES: OCCOc1cc(-n2cnnc2-c2cc3cc(C(F)(F)F)cc(F)c3[nH]2)c(OC(F)(F)F)cn1] | 411 |
| 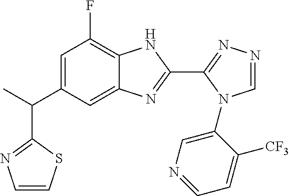 [SMILES: CC(c1cc(F)c2[nH]c(-c3nncn3-c3cnccc3C(F)(F)F)nc2c1)c1nccs1] | 412 |
| 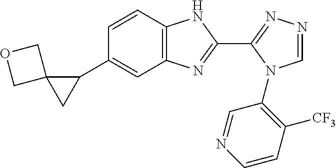 [SMILES: FC(F)(F)c1ccncc1-n1cnnc1-c1nc2cc(C3CC34COC4)ccc2[nH]1] | 413 |
| 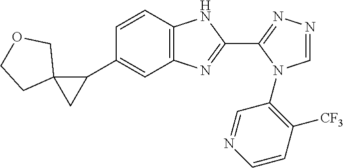 [SMILES: FC(F)(F)c1ccncc1-n1cnnc1-c1nc2cc(C3CC34CCOC4)ccc2[nH]1] | 414 |
| 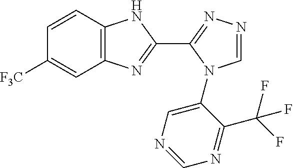 [SMILES: FC(F)(F)c1ccc2[nH]c(-c3nncn3-c3cncnc3C(F)(F)F)nc2c1] | 415 |
| 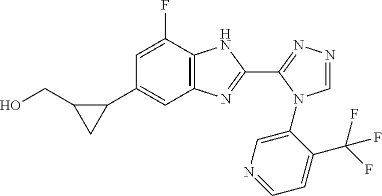 [SMILES: OCC1CC1c1cc(F)c2[nH]c(-c3nncn3-c3cnccc3C(F)(F)F)nc2c1] | 416 |
| 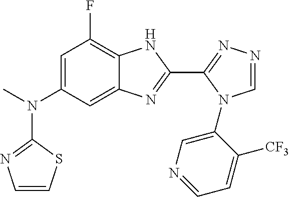 [SMILES: CN(c1cc(F)c2[nH]c(-c3nncn3-c3cnccc3C(F)(F)F)nc2c1)c1nccs1] | 417 |
| 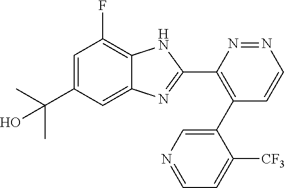 [SMILES: CC(C)(O)c1cc(F)c2[nH]c(-c3nnccc3-c3cnccc3C(F)(F)F)nc2c1] | 418 |
| 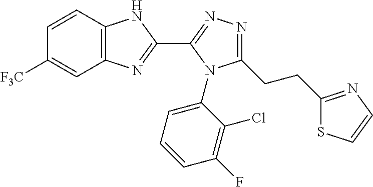 [SMILES: Fc1cccc(-n2c(CCc3nccs3)nnc2-c2nc3cc(C(F)(F)F)ccc3[nH]2)c1Cl] | 419 |
| 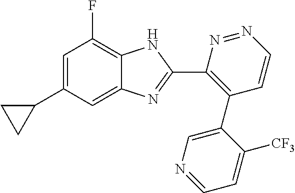 [SMILES: Fc1cc(C2CC2)cc2nc(-c3nnccc3-c3cnccc3C(F)(F)F)[nH]c12] | 420 |
| 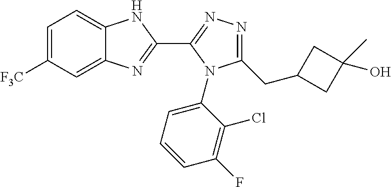 [SMILES: CC1(O)CC(Cc2nnc(-c3nc4cc(C(F)(F)F)ccc4[nH]3)n2-c2cccc(F)c2Cl)C1] | 421 |
| 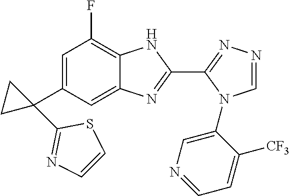 [SMILES: Fc1cc(C2(c3nccs3)CC2)cc2nc(-c3nncn3-c3cnccc3C(F)(F)F)[nH]c12] | 422 |
| 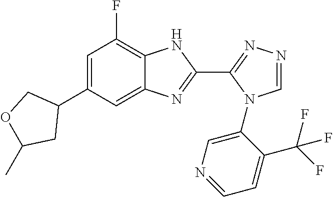 [SMILES: CC1CC(c2cc(F)c3[nH]c(-c4nncn4-c4cnccc4C(F)(F)F)nc3c2)CO1] | 423 |
| 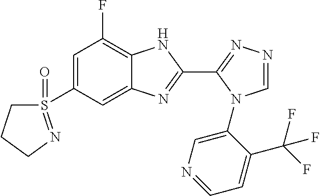 [SMILES: O=S1(c2cc(F)c3[nH]c(-c4nncn4-c4cnccc4C(F)(F)F)nc3c2)=NCCC1] | 424 |
| 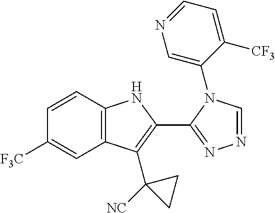 [SMILES: N#CC1(c2c(-c3nncn3-c3cnccc3C(F)(F)F)[nH]c3ccc(C(F)(F)F)cc23)CC1] | 425 |
| 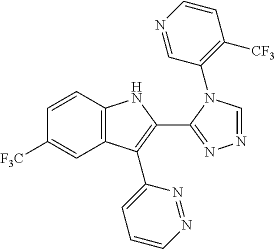 [SMILES: FC(F)(F)c1ccc2[nH]c(-c3nncn3-c3cnccc3C(F)(F)F)c(-c3cccnn3)c2c1] | 426 |
| 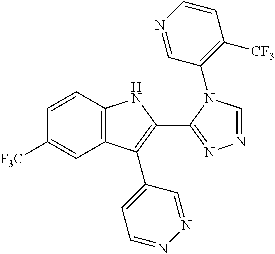 [SMILES: FC(F)(F)c1ccc2[nH]c(-c3nncn3-c3cnccc3C(F)(F)F)c(-c3ccnnc3)c2c1] | 427 |
| 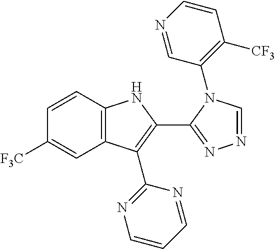 [SMILES: FC(F)(F)c1ccc2[nH]c(-c3nncn3-c3cnccc3C(F)(F)F)c(-c3ncccn3)c2c1] | 428 |
| 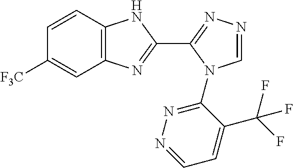 [SMILES: FC(F)(F)c1ccc2[nH]c(-c3nncn3-c3nnccc3C(F)(F)F)nc2c1] | 429 |
| 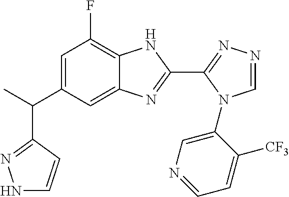 [SMILES: CC(c1cc(F)c2[nH]c(-c3nncn3-c3cnccc3C(F)(F)F)nc2c1)c1cc[nH]n1] | 430 |
| 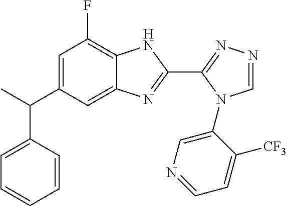 [SMILES: CC(c1ccccc1)c1cc(F)c2[nH]c(-c3nncn3-c3cnccc3C(F)(F)F)nc2c1] | 431 |
| 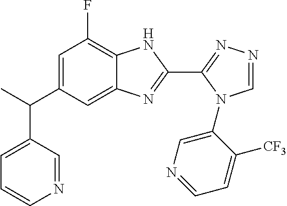 [SMILES: CC(c1cccnc1)c1cc(F)c2[nH]c(-c3nncn3-c3cnccc3C(F)(F)F)nc2c1] | 432 |
| 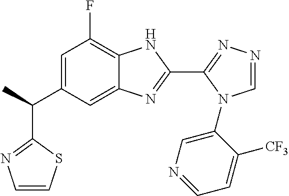 [SMILES: C[C@@H](c1cc(F)c2[nH]c(-c3nncn3-c3cnccc3C(F)(F)F)nc2c1)c1nccs1] | 433 |
| 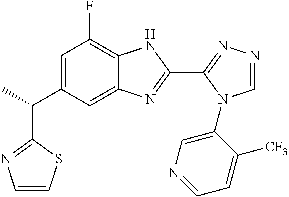 [SMILES: C[C@H](c1cc(F)c2[nH]c(-c3nncn3-c3cnccc3C(F)(F)F)nc2c1)c1nccs1] | 434 |
| 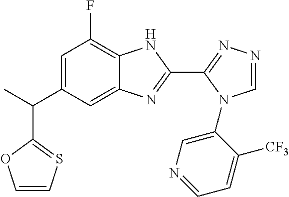 | 435 |
| 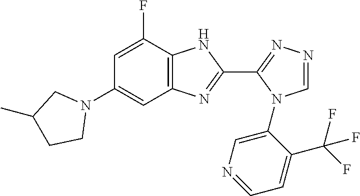 [SMILES: CC1CCN(c2cc(F)c3[nH]c(-c4nncn4-c4cnccc4C(F)(F)F)nc3c2)C1] | 436 |
| 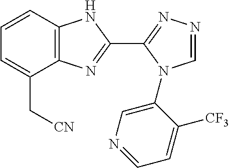 [SMILES: N#CCc1cccc2[nH]c(-c3nncn3-c3cnccc3C(F)(F)F)nc12] | 437 |
| 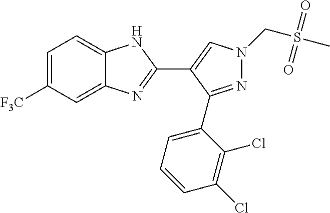 [SMILES: CS(=O)(=O)Cn1cc(-c2nc3cc(C(F)(F)F)ccc3[nH]2)c(-c2cccc(Cl)c2Cl)n1] | 438 |
| 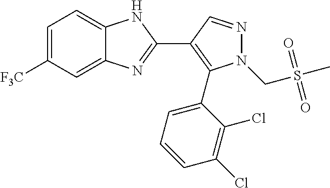 [SMILES: CS(=O)(=O)Cn1ncc(-c2nc3cc(C(F)(F)F)ccc3[nH]2)c1-c1cccc(Cl)c1Cl] | 439 |
| 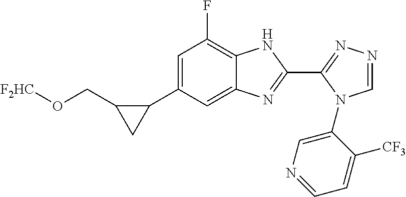 [SMILES: Fc1cc(C2CC2COC(F)F)cc2nc(-c3nncn3-c3cnccc3C(F)(F)F)[nH]c12] | 440 |
| 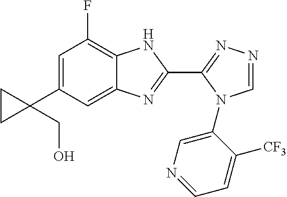 [SMILES: OCC1(c2cc(F)c3[nH]c(-c4nncn4-c4cnccc4C(F)(F)F)nc3c2)CC1] | 441 |
| 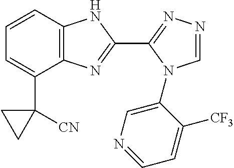 [SMILES: N#CC1(c2cccc3[nH]c(-c4nncn4-c4cnccc4C(F)(F)F)nc23)CC1] | 442 |
| 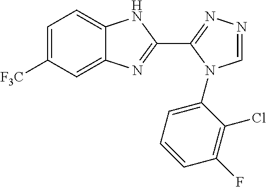 [SMILES: Fc1cccc(-n2cnnc2-c2nc3cc(C(F)(F)F)ccc3[nH]2)c1Cl] | 443 |
| 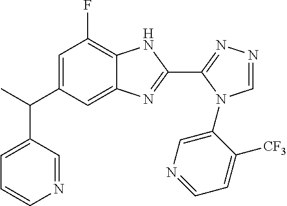 [SMILES: CC(c1cccnc1)c1cc(F)c2[nH]c(-c3nncn3-c3cnccc3C(F)(F)F)nc2c1] | 444 |
| 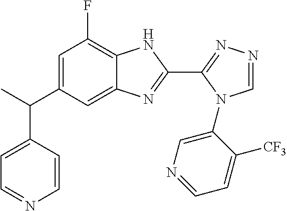 [SMILES: CC(c1ccncc1)c1cc(F)c2[nH]c(-c3nncn3-c3cnccc3C(F)(F)F)nc2c1] | 445 |
| 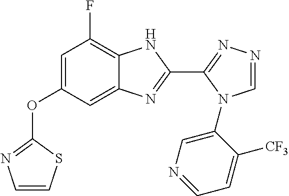 [SMILES: Fc1cc(Oc2nccs2)cc2nc(-c3nncn3-c3cnccc3C(F)(F)F)[nH]c12] | 446 |
| 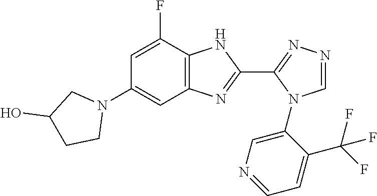 [SMILES: OC1CCN(c2cc(F)c3[nH]c(-c4nncn4-c4cnccc4C(F)(F)F)nc3c2)C1] | 447 |
| 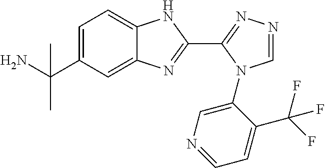 [SMILES: CC(C)(N)c1ccc2[nH]c(-c3nncn3-c3cnccc3C(F)(F)F)nc2c1] | 448 |
| 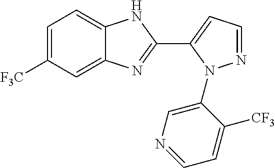 [SMILES: FC(F)(F)c1ccc2[nH]c(-c3ccnn3-c3cnccc3C(F)(F)F)nc2c1] | 449 |
| 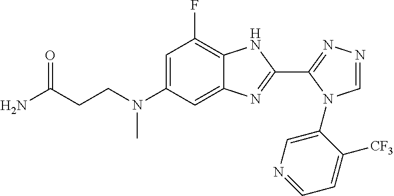 [SMILES: CN(CCC(N)=O)c1cc(F)c2[nH]c(-c3nncn3-c3cnccc3C(F)(F)F)nc2c1] | 450 |
| 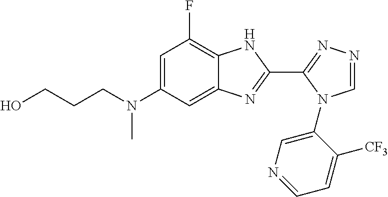 [SMILES: CN(CCCO)c1cc(F)c2[nH]c(-c3nncn3-c3cnccc3C(F)(F)F)nc2c1] | 451 |
| 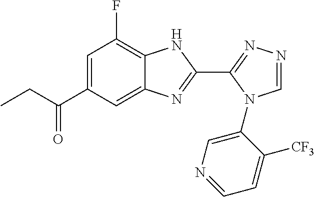 [SMILES: CCC(=O)c1cc(F)c2[nH]c(-c3nncn3-c3cnccc3C(F)(F)F)nc2c1] | 452 |
| 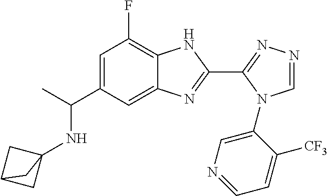 [SMILES: CC(NC12CC(C1)C2)c1cc(F)c2[nH]c(-c3nncn3-c3cnccc3C(F)(F)F)nc2c1] | 453 |
| 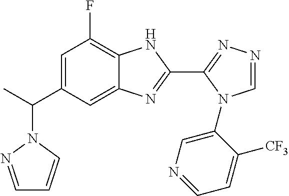 [SMILES: CC(c1cc(F)c2[nH]c(-c3nncn3-c3cnccc3C(F)(F)F)nc2c1)n1cccn1] | 454 |
| 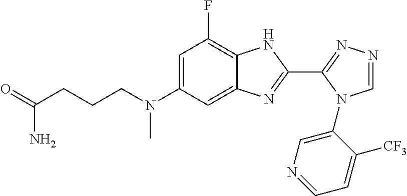 [SMILES: CN(CCCC(N)=O)c1cc(F)c2[nH]c(-c3nncn3-c3cnccc3C(F)(F)F)nc2c1] | 455 |
| 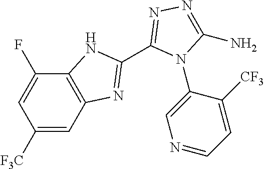 [SMILES: Nc1nnc(-c2nc3cc(C(F)(F)F)cc(F)c3[nH]2)n1-c1cnccc1C(F)(F)F] | 456 |
| 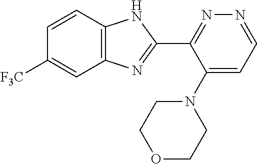 [SMILES: FC(F)(F)c1ccc2[nH]c(-c3nnccc3N3CCOCC3)nc2c1] | 457 |
| 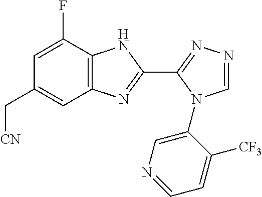 [SMILES: N#CCc1cc(F)c2[nH]c(-c3nncn3-c3cnccc3C(F)(F)F)nc2c1] | 458 |
| 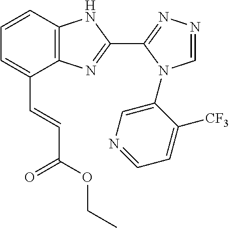 [SMILES: CCOC(=O)/C=C/c1cccc2[nH]c(-c3nncn3-c3cnccc3C(F)(F)F)nc12] | 459 |
| 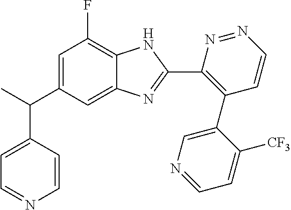 [SMILES: CC(c1ccncc1)c1cc(F)c2[nH]c(-c3nnccc3-c3cnccc3C(F)(F)F)nc2c1] | 460 |
| 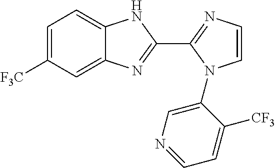 [SMILES: FC(F)(F)c1ccc2[nH]c(-c3nccn3-c3cnccc3C(F)(F)F)nc2c1] | 461 |
| 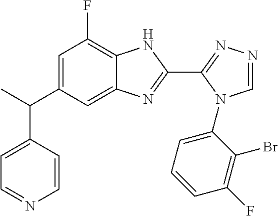 [SMILES: CC(c1ccncc1)c1cc(F)c2[nH]c(-c3nncn3-c3cccc(F)c3Br)nc2c1] | 462 |
| 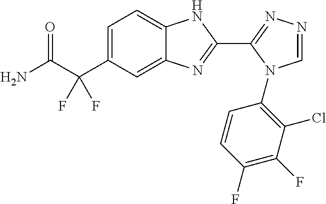 [SMILES: NC(=O)C(F)(F)c1ccc2[nH]c(-c3nncn3-c3ccc(F)c(F)c3Cl)nc2c1] | 463 |
| 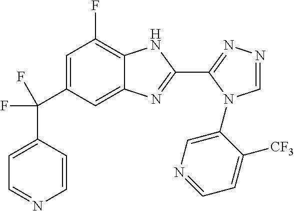 [SMILES: Fc1cc(C(F)(F)c2ccncc2)cc2nc(-c3nncn3-c3cnccc3C(F)(F)F)[nH]c12] | 464 |
| 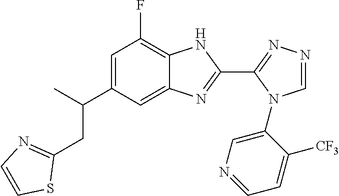 [SMILES: CC(Cc1nccs1)c1cc(F)c2[nH]c(-c3nncn3-c3cnccc3C(F)(F)F)nc2c1] | 465 |
| 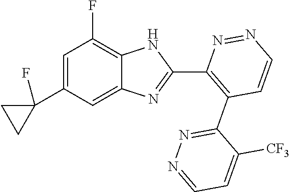 [SMILES: Fc1cc(C2(F)CC2)cc2nc(-c3nnccc3-c3nnccc3C(F)(F)F)[nH]c12] | 466 |
| 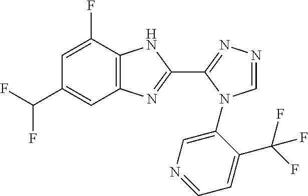 [SMILES: Fc1cc(C(F)F)cc2nc(-c3nncn3-c3cnccc3C(F)(F)F)[nH]c12] | 467 |
| 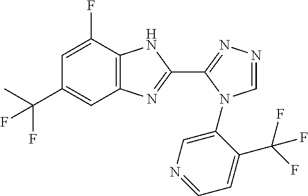 [SMILES: CC(F)(F)c1cc(F)c2[nH]c(-c3nncn3-c3cnccc3C(F)(F)F)nc2c1] | 468 |

Another aspect of the disclosure provides a pharmaceutical composition comprising at least one compound selected from compounds of Formulae 1, 2, 3-1, 3-2, 3-3, 4, 5, 6-1, 6-2, 7-1, 7-2, 8-1, 8-2, 8-3, 8-4, 8-5, 8-6, 9-1, 9-2, 9-3, 9-4, 10-1, 10-2, 10-3, 10-4, 10-5, 10-6, 11-1, 11-2, 11-3, 11-4, 11-5, 11-6, 11-7, 12-1, 12-2, 12-3, 12-4, 12-5, 12-6, 12-7, and 12-8 (e.g., Compounds 1 to 468) disclosed herein, a tautomer thereof, a solvate or stereoisomer of the compound or the tautomer, or a pharmaceutically acceptable salt of the foregoing, and at least one pharmaceutically acceptable carrier.

In some embodiments, the pharmaceutically acceptable carrier is selected from pharmaceutically acceptable vehicles and pharmaceutically acceptable adjuvants. In some embodiments, the pharmaceutically acceptable carrier is chosen from pharmaceutically acceptable fillers, disintegrants, surfactants, binders, and lubricants.

It will also be appreciated that a pharmaceutical composition of this disclosure can be employed in combination therapies; that is, the pharmaceutical compositions described herein can further include an additional active pharmaceutical agent. Alternatively, a pharmaceutical composition comprising a compound selected from compounds of Formulae 1, 2, 3-1, 3-2, 3-3, 4,5, 6-1, 6-2, 7-1, 7-2, 8-1, 8-2, 8-3, 8-4, 8-5, 8-6, 9-1, 9-2, 9-3, 9-4, 10-1, 10-2, 10-3, 10-4, 10-5, 10-6, 11-1, 11-2, 11-3, 11-4, 11-5, 11-6, 11-7, 12-1, 12-2, 12-3, 12-4, 12-5, 12-6, 12-7, and 12-8 (e.g., Compounds 1 to 468), a tautomer thereof, a solvate or stereoisomer of the compound or the tautomer, or a pharmaceutically acceptable salt of the foregoing can be administered as a separate composition concurrently with, prior to, or subsequent to, a composition comprising an additional active pharmaceutical agent.

In some embodiments, the pharmaceutically acceptable carrier may be chosen from adjuvants and vehicles. The pharmaceutically acceptable carrier, as used herein, can be chosen, for example, from any and all solvents, diluents, other liquid vehicles, dispersion aids, suspension aids, surface active agents, isotonic agents, thickening agents, emulsifying agents, preservatives, solid binders, and lubricants, which are suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988 to 1999, Marcel Dekker, New York discloses various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

A compound selected from compounds of Formulae 1, 2, 3-1, 3-2, 3-3, 4, 5, 6-1, 6-2, 7-1, 7-2, 8-1, 8-2, 8-3, 8-4, 8-5, 8-6, 9-1, 9-2, 9-3, 9-4, 10-1, 10-2, 10-3, 10-4, 10-5, 10-6, 11-1, 11-2, 11-3, 11-4, 11-5, 11-6, 11-7, 12-1, 12-2, 12-3, 12-4, 12-5, 12-6, 12-7, and 12-8 (e.g., Compounds 1 to 468) disclosed herein, a tautomer thereof, a solvate or stereoisomer of the compound or the tautomer, or a pharmaceutically acceptable salt of the foregoing, or a pharmaceutical composition disclosed herein can be administered orally in solid dosage forms, such as capsules, tablets, troches, dragées, granules and powders, or in liquid dosage forms, such as elixirs, syrups, emulsions, dispersions, and suspensions. The compound, tautomer, solvate, stereoisomer, or pharmaceutically acceptable salt described herein can also be administered parenterally, in sterile liquid dosage forms, such as dispersions, suspensions or solutions. Other dosages forms that can also be used to administer the compound, tautomer, solvate, stereoisomer, or pharmaceutically acceptable salt described herein as an ointment, cream, drops, transdermal patch or powder for topical administration, as an ophthalmic solution or suspension formation, e.g., eye drops, for ocular administration, as an aerosol spray or powder composition for inhalation or intranasal administration, or as a cream, ointment, spray or suppository for rectal or vaginal administration.

Gelatin capsules containing a compound, a tautomer thereof, a solvate or stereoisomer of the compound or the tautomer, and/or a pharmaceutically acceptable salt of the foregoing disclosed herein and powdered carriers, such as lactose, starch, cellulose derivatives, magnesium stearate, stearic acid, and the like, can also be used. Similar diluents can be used to make compressed tablets. Both tablets and capsules can be manufactured as sustained release products to provide for continuous release of medication over a period of time. Compressed tablets can be sugar coated or film coated to mask any unpleasant taste and protect the tablet from the atmosphere, or enteric coated for selective disintegration in the gastrointestinal tract.

Liquid dosage forms for oral administration can further comprise at least one agent selected from coloring and flavoring agents to increase patient acceptance.

In general, water, a suitable oil, saline, aqueous dextrose (glucose), and related sugar solutions and glycols such as propylene glycol or polyethylene glycols can be examples of suitable carriers for parenteral solutions. Solutions for parenteral administration may comprise a water-soluble salt of the at least one compound describe herein, at least one suitable stabilizing agent, and if necessary, at least one buffer substance. Antioxidizing agents such as sodium bisulfite, sodium sulfite, or ascorbic acid, either alone or combined, can be examples of suitable stabilizing agents. Citric acid and its salts and sodium EDTA can also be used as examples of suitable stabilizing agents. In addition, parenteral solutions can further comprise at least one preservative, selected, for example, from benzalkonium chloride, methyl- and propylparaben, and chlorobutanol.

A pharmaceutically acceptable carrier is, for example, selected from carriers that are compatible with active ingredients of the composition (and in some embodiments, capable of stabilizing the active ingredients) and not deleterious to the subject to be treated. For example, solubilizing agents, such as cyclodextrins (which can form specific, more soluble complexes with the at least one compound and/or at least one pharmaceutically acceptable salt disclosed herein), can be utilized as pharmaceutical excipients for delivery of the active ingredients. Examples of other carriers include colloidal silicon dioxide, magnesium stearate, cellulose, sodium lauryl sulfate, and pigments such as D&C Yellow #10. Suitable pharmaceutically acceptable carriers are described in Remington's Pharmaceutical Sciences, A. Osol.

For administration by inhalation, the compound, tautomer, solvate, stereoisomer, or pharmaceutically acceptable salt described herein may be conveniently delivered in the form of an aerosol spray presentation from pressurized packs or nebulisers. The compound, tautomer, solvate, stereoisomer, or pharmaceutically acceptable salt described herein may also be delivered as powders, which may be formulated, and the powder composition may be inhaled with the aid of an insufflation powder inhaler device. One exemplary delivery system for inhalation can be metered dose inhalation (MDI) aerosol, which may be formulated as a suspension or solution of a compound, tautomer, solvate, stereoisomer, or pharmaceutically acceptable salt described herein in at least one suitable propellant, selected, for example, from fluorocarbons and hydrocarbons.

For ocular administration, an ophthalmic preparation may be formulated with an appropriate weight percentage of a solution or suspension of the compound, tautomer, solvate, stereoisomer, or pharmaceutically acceptable salt described herein in an appropriate ophthalmic vehicle, such that the compound, tautomer, solvate, stereoisomer, or pharmaceutically acceptable salt described herein is maintained in contact with the ocular surface for a sufficient time period to allow the compound to penetrate the corneal and internal regions of the eye.

Useful pharmaceutical dosage-forms for administration of the compound, tautomer, solvate, stereoisomer, or pharmaceutically acceptable salt described herein include, but are not limited to, hard and soft gelatin capsules, tablets, parenteral injectables, and oral suspensions. In some embodiments, the pharmaceutical compositions disclosed herein may be in the form of controlled release or sustained release compositions as known in the art.

The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules, lozenges or the like in the case of solid compositions. In such compositions, the active material is usually a component ranging from about 0.1 to about 50% by weight or preferably from about 1 to about 40% by weight with the remainder being various vehicles or carriers and processing aids helpful for forming the desired dosing form. Unit dosage formulations are preferably about of 5, 10, 25, 50, 100, 250, 500, or 1,000 mg per unit. In a particular embodiment, unit dosage forms are packaged in a multipack adapted for sequential use, such as blisterpack comprising sheets of at least 6, 9 or 12 unit dosage forms.

In some embodiments, unit capsules can be prepared by filling standard two-piece hard gelatin capsules each with, for example, 100 milligrams of the compound, tautomer, solvate, stereoisomer, or pharmaceutically acceptable salt described herein in powder, 150 milligrams of lactose, 50 milligrams of cellulose, and 6 milligrams magnesium stearate.

In some embodiments, a mixture of the compound, tautomer, solvate, stereoisomer, or pharmaceutically acceptable salt described herein and a digestible oil such as soybean oil, cottonseed oil or olive oil can be prepared and injected by means of a positive displacement pump into gelatin to form soft gelatin capsules containing 100 milligrams of the active ingredient. The capsules are washed and dried.

In some embodiments, tablets can be prepared by conventional procedures so that the dosage unit comprises, for example, 100 milligrams of the compound, stereoisomers thereof, or pharmaceutically acceptable salts thereof, 0.2 milligrams of colloidal silicon dioxide, milligrams of magnesium stearate, 275 milligrams of microcrystalline cellulose, 11 milligrams of starch and 98.8 milligrams of lactose. Appropriate coatings may be applied to increase palatability or delay absorption.

In some embodiments, a parenteral composition suitable for administration by injection can be prepared by stirring 1.5% by weight of the compound and/or at least an enantiomer, a diastereoisomer, or pharmaceutically acceptable salt thereof disclosed herein in 10% by volume propylene glycol. The solution is made to the expected volume with water for injection and sterilized.

In some embodiment, an aqueous suspension can be prepared for oral administration. For example, each 5 milliliters of an aqueous suspension comprising 100 milligrams of finely divided compound, stereoisomers thereof, or pharmaceutically acceptable salts thereof, 100 milligrams of sodium carboxymethyl cellulose, 5 milligrams of sodium benzoate, 1.0 grams of sorbitol solution, U.S.P., and 0.025 milliliters of vanillin can be used.

The same dosage forms can generally be used when the compound, tautomer, solvate, stereoisomer, or pharmaceutically acceptable salt described herein is administered stepwise or in conjunction with at least one other therapeutic agent. When drugs are administered in physical combination, the dosage form and administration route should be selected depending on the compatibility of the combined drugs. Thus, the term coadministration is understood to include the administration of at least two agents concomitantly or sequentially, or alternatively as a fixed dose combination of the at least two active components.

The compound, tautomer, solvate, stereoisomer, or pharmaceutically acceptable salt disclosed herein can be administered as the sole active ingredient or in combination with at least one second active ingredient.

The compound, tautomer, solvate, or stereoisomer described herein may be used in the aforementioned form or in the form of their pharmaceutically acceptable salts, such as hydrochlorides, hydrobromides, acetates, sulfates, citrates, carbonates, trifluoroacetates and the like. When the compound, tautomer, solvate, stereoisomer, or pharmaceutically acceptable salt described herein contain relatively acidic functionalities, salts can be obtained by addition of the desired base, either neat or in a suitable inert solvent. Examples of pharmaceutically acceptable base addition salts include sodium, potassium, calcium, ammonium, organic amino, or magnesium salts, or the like. When the compound, tautomer, solvate, or stereoisomer described herein contain relatively basic functionalities, salts can be obtained by addition of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, for example, Berge et al., “Pharmaceutical Salts,” Journal of Pharmaceutical Science, 1977, 66, 1-19).

Neutral forms of the pharmaceutically acceptable salt described herein may be regenerated by contacting the salt with a base or acid, and isolating the parent compound in the conventional manner.

This disclosure provides prodrugs. Prodrugs of the compound, tautomer, solvate, stereoisomer, or pharmaceutically acceptable salt described herein that readily undergo chemical changes under physiological conditions to provide the compound, tautomer, solvate, stereoisomer, or pharmaceutically acceptable salt of the present disclosure. Additionally, prodrugs can be converted to the compound, tautomer, solvate, stereoisomer, or a pharmaceutically acceptable salt of the present disclosure by chemical or biochemical methods in an ex vivo environment. For example, prodrugs can be slowly converted to the compound, tautomer, solvate, stereoisomer, or pharmaceutically acceptable salt of the present disclosure when placed in a transdermal patch reservoir with a suitable enzyme or chemical reagent. Prodrugs are often useful because, in some situations, they may be easier to administer than the parent drug. They may, for instance, be more bioavailable by oral administration than the parent drug. The prodrug may also have improved solubility in pharmacological compositions over the parent drug. A wide variety of prodrug derivatives are known in the art, such as those that rely on hydrolytic cleavage or oxidative activation of the prodrug. An example, without limitation, of a prodrug would be a compound of the present disclosure which is administered as an ester (the “prodrug”), but then is metabolically hydrolyzed to the carboxylic acid, i.e., the active entity.

Certain compound, tautomer, stereoisomer, or pharmaceutically acceptable salt of the disclosure can exist in unsolvated forms as well as solvated forms, including hydrate forms. Certain compound, tautomer, solvate, stereoisomer, or pharmaceutically acceptable salt of the disclosure may exist in multiple crystalline or amorphous forms.

Certain compound, tautomer, solvate, or pharmaceutically acceptable salt in this disclosure possesses asymmetric carbon atoms (optical centers) or double bonds; the racemates, enantiomers, diastereoisomers, geometric isomers and individual isomers are all intended to be encompassed within the scope of the present disclosure.

### III. Methods of Treatment and Uses

The present disclosure provides methods of treatment and uses utilizing a compound set forth in any one of the various embodiments of Section II (Compounds and Compositions) and Table 1, e.g., a compound of Formula 1, 2, 3-1, 3-2, 3-3, 4, 5, 6-1, 6-2, 7-1, 7-2, 8-1, 8-2, 8-3, 8-4, 8-5, 8-6, 9-1, 9-2, 9-3, 9-4, 10-1, 10-2, 10-3, 10-4, 10-5, 10-6, 11-1, 11-2, 11-3, 11-4, 11-5, 11-6, 11-7, 12-1, 12-2, 12-3, 12-4, 12-5, 12-6, 12-7, or 12-8, as well as Compounds 1 to 468 in Table 1, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt.

One aspect of the disclosure provides a method of treating a disease or condition, comprising administering to a subject in need thereof, a therapeutically effective amount of a compound of Formula 1, 2, 3-1, 3-2, 3-3, 4, 5, 6-1, 6-2, 7-1, 7-2, 8-1, 8-2, 8-3, 8-4, 8-5, 8-6, 9-1, 9-2, 9-3, 9-4, 10-1, 10-2, 10-3, 10-4, 10-5, 10-6, 11-1, 11-2, 11-3, 11-4, 11-5, 11-6, 11-7, 12-1, 12-2, 12-3, 12-4, 12-5, 12-6, 12-7, or 12-8 (e.g., Compounds 1 to 468) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt, wherein the disease or condition includes, but is not limited to, amyotrophic lateral sclerosis (ALS), Parkinson's disease, Parkinsonian syndromes, ischemia, stroke, herpes infection, a demyelinating disease such as multiple sclerosis, traumatic brain injury, sepsis, a chronic disease of PNS including inherited neuropathies, such as, but is not limited to Charcot-Marie-Tooth disease and chronic inflammatory demyelinating polyneuropathy (CIDP), an optic nerve disorder such as glaucoma, and retinal ganglion degeneration, colitis, a metabolic disease or disorder such as diabetic neuropathy, nonalcoholic fatty liver disease (NAFLD) and nonalcoholic steatohepatitis (NASH) and a peripheral neuropathy like CIPN induced by various drugs, such as, but not limited to taxanes, vinca alkaloids and proteasome inhibitors. In some embodiments, the disease or condition is caused by or associated with axonal degeneration or neuronal cells damage.

In another aspect, disclosed herein is a compound, tautomer, solvate, stereoisomer, or pharmaceutically acceptable salt as described herein, including a compound of Formula 1, 2, 3-1, 3-2, 3-3, 4, 5, 6-1, 6-2, 7-1, 7-2, 8-1, 8-2, 8-3, 8-4, 8-5, 8-6, 9-1, 9-2, 9-3, 9-4, 10-1, 10-2, 10-3, 10-4, 10-5, 10-6, 11-1, 11-2, 11-3, 11-4, 11-5, 11-6, 11-7, 12-1, 12-2, 12-3, 12-4, 12-5, 12-6, 12-7, or 12-8 (e.g., Compounds 1 to 468) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt, for use as a medicament.

In another aspect, disclosed herein is use of a compound, tautomer, solvate, stereoisomer, or pharmaceutically acceptable salt as described herein, including a compound of Formula 1, 2, 3-1, 3-2, 3-3, 4, 5, 6-1, 6-2, 7-1, 7-2, 8-1, 8-2, 8-3, 8-4, 8-5, 8-6, 9-1, 9-2, 9-3, 9-4, 10-1, 10-2, 10-3, 10-4, 10-5, 10-6, 11-1, 11-2, 11-3, 11-4, 11-5, 11-6, 11-7, 12-1, 12-2, 12-3, 12-4, 12-5, 12-6, 12-7, or 12-8 (e.g., Compounds 1 to 468) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt, for the manufacture of a medicament for treating a disease or condition that includes, but is not limited to, amyotrophic lateral sclerosis (ALS), Parkinson's disease, Parkinsonian syndromes, ischemia, stroke, herpes infection, a demyelinating disease such as multiple sclerosis, traumatic brain injury, sepsis, a chronic disease of PNS including inherited neuropathies, such as, but is not limited to Charcot-Marie-Tooth disease and chronic inflammatory demyelinating polyneuropathy (CIDP), an optic nerve disorder such as glaucoma, and retinal ganglion degeneration, colitis, a metabolic disease or disorder such as diabetic neuropathy, nonalcoholic fatty liver disease (NAFLD) and nonalcoholic steatohepatitis (NASH) and a peripheral neuropathy like CIPN induced by various drugs, such as, but is not limited to taxanes, vinca alkaloids and proteasome inhibitors. In some embodiments, the disease or condition is caused by or associated with axonal degeneration or neuronal cell damage.

In a further aspect of this disclosure, a compound, tautomer, solvate, stereoisomer, or pharmaceutically acceptable salt as described herein, including a compound of Formula 1, 2, 3-1, 3-2, 3-3, 4, 5, 6-1, 6-2, 7-1, 7-2, 8-1, 8-2, 8-3, 8-4, 8-5, 8-6, 9-1, 9-2, 9-3, 9-4, 10-1, 10-2, 10-3, 10-4, 10-5, 10-6, 11-1, 11-2, 11-3, 11-4, 11-5, 11-6, 11-7, 12-1, 12-2, 12-3, 12-4, 12-5, 12-6, 12-7, or 12-8 (e.g., Compounds 1 to 468) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt, is for use in treating a disease or condition that includes, but is not limited to, amyotrophic lateral sclerosis (ALS), Parkinson's disease, Parkinsonian syndromes, ischemia, stroke, herpes infection, a demyelinating disease such as multiple sclerosis, traumatic brain injury, sepsis, a chronic disease of PNS including inherited neuropathies, such as, but is not limited to Charcot-Marie-Tooth disease and chronic inflammatory demyelinating polyneuropathy (CIDP), an optic nerve disorder such as glaucoma, and retinal ganglion degeneration, colitis, a metabolic disease or disorder such as diabetic neuropathy, nonalcoholic fatty liver disease (NAFLD) and nonalcoholic steatohepatitis (NASH) and a peripheral neuropathy like CIPN induced by various drugs, such as, but is not limited to taxanes, vinca alkaloids and proteasome inhibitors. In some embodiments, the disease or condition is caused by or associated with axonal degeneration or neuronal cell damage.

Another aspect of the disclosure provides a method of inhibiting or preventing axonal degeneration, comprising administering to a subject in need thereof, a therapeutically effective amount of a compound of Formula 1, 2, 3-1, 3-2, 3-3, 4, 5, 6-1, 6-2, 7-1, 7-2, 8-1, 8-2, 8-3, 8-4, 8-5, 8-6, 9-1, 9-2, 9-3, 9-4, 10-1, 10-2, 10-3, 10-4, 10-5, 10-6, 11-1, 11-2, 11-3, 11-4, 11-5, 11-6, 11-7, 12-1, 12-2, 12-3, 12-4, 12-5, 12-6, 12-7, or 12-8 (e.g., Compounds 1 to 468) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt.

In another aspect, disclosed herein is use of a compound, tautomer, solvate, stereoisomer, or pharmaceutically acceptable salt as described herein, including a compound of Formula 1, 2, 3-1, 3-2, 3-3, 4, 5, 6-1, 6-2, 7-1, 7-2, 8-1, 8-2, 8-3, 8-4, 8-5, 8-6, 9-1, 9-2, 9-3, 9-4, 10-1, 10-2, 10-3, 10-4, 10-5, 10-6, 11-1, 11-2, 11-3, 11-4, 11-5, 11-6, 11-7, 12-1, 12-2, 12-3, 12-4, 12-5, 12-6, 12-7, or 12-8 (e.g., Compounds 1 to 468) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt, for the manufacture of a medicament for inhibiting or preventing axonal degeneration or neuronal cells damage.

In a further aspect of this disclosure, a compound, tautomer, solvate, stereoisomer, or pharmaceutically acceptable salt as described herein, including a compound of Formula 1, 2, 3-1, 3-2, 3-3, 4, 5, 6-1, 6-2, 7-1, 7-2, 8-1, 8-2, 8-3, 8-4, 8-5, 8-6, 9-1, 9-2, 9-3, 9-4, 10-1, 10-2, 10-3, 10-4, 10-5, 10-6, 11-1, 11-2, 11-3, 11-4, 11-5, 11-6, 11-7, 12-1, 12-2, 12-3, 12-4, 12-5, 12-6, 12-7, or 12-8 (e.g., Compounds 1 to 468) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt, is for use in inhibiting or preventing axonal degeneration or neuronal cells damage.

Another aspect of the disclosure provides a method of modulating, e.g., inhibiting, SARM1 in a subject in need thereof, comprising administering to the subject, a therapeutically effective amount of a compound of Formula 1, 2, 3-1, 3-2, 3-3, 4, 5, 6-1, 6-2, 7-1, 7-2, 8-1, 8-2, 8-3, 8-4, 8-5, 8-6, 9-1, 9-2, 9-3, 9-4, 10-1, 10-2, 10-3, 10-4, 10-5, 10-6, 11-1, 11-2, 11-3, 11-4, 11-5, 11-6, 11-7, 12-1, 12-2, 12-3, 12-4, 12-5, 12-6, 12-7, or 12-8 (e.g., Compounds 1 to 468) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt.

In another aspect, disclosed herein is use of a compound, tautomer, solvate, stereoisomer, or pharmaceutically acceptable salt as described herein, including a compound of Formula 1, 2, 3-1, 3-2, 3-3, 4, 5, 6-1, 6-2, 7-1, 7-2, 8-1, 8-2, 8-3, 8-4, 8-5, 8-6, 9-1, 9-2, 9-3, 9-4, 10-1, 10-2, 10-3, 10-4, 10-5, 10-6, 11-1, 11-2, 11-3, 11-4, 11-5, 11-6, 11-7, 12-1, 12-2, 12-3, 12-4, 12-5, 12-6, 12-7, or 12-8 (e.g., Compounds 1 to 468) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt, for modulating, e.g., inhibiting, SARM1 in a subject in need thereof.

In another aspect of this disclosure, a compound, tautomer, a solvate or stereoisomer of the compound or the tautomer, or pharmaceutically acceptable salt as described herein, including a compound of Formula 1, 2, 3-1, 3-2, 3-3, 4, 5, 6-1, 6-2, 7-1, 7-2, 8-1, 8-2, 8-3, 8-4, 8-5, 8-6, 9-1, 9-2, 9-3, 9-4, 10-1, 10-2, 10-3, 10-4, 10-5, 10-6, 11-1, 11-2, 11-3, 11-4, 11-5, 11-6, 11-7, 12-1, 12-2, 12-3, 12-4, 12-5, 12-6, 12-7, or 12-8 (e.g., Compounds 1 to 468) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt, is for use in modulating, e.g., inhibiting, SARM1 in a subject in need thereof by contacting the subject with the compound, tautomer, a solvate or stereoisomer of the compound or the tautomer, pharmaceutically acceptable salt, or pharmaceutical composition.

A compound of Formula 1, 2, 3-1, 3-2, 3-3, 4, 5, 6-1, 6-2, 7-1, 7-2, 8-1, 8-2, 8-3, 8-4, 8-5, 8-6, 9-1, 9-2, 9-3, 9-4, 10-1, 10-2, 10-3, 10-4, 10-5, 10-6, 11-1, 11-2, 11-3, 11-4, 11-5, 11-6, 11-7, 12-1, 12-2, 12-3, 12-4, 12-5, 12-6, 12-7, or 12-8 (e.g., Compounds 1 to 468) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt may be administered once daily, twice daily, or three times daily, for example, for the treatment of a disease or condition, that includes, but is not limited to, amyotrophic lateral sclerosis (ALS), Parkinson's disease, Parkinsonian syndromes, ischemia, stroke, herpes infection, a demyelinating disease such as multiple sclerosis, traumatic brain injury, sepsis, a chronic disease of PNS including inherited neuropathies, such as, but is not limited to Charcot-Marie-Tooth disease and chronic inflammatory demyelinating polyneuropathy (CIDP), an optic nerve disorder such as glaucoma, and retinal ganglion degeneration, colitis, a metabolic disease or disorder such as diabetic neuropathy, nonalcoholic fatty liver disease (NAFLD) and nonalcoholic steatohepatitis (NASH) and a peripheral neuropathy like CIPN induced by various drugs, such as, but is not limited to taxanes, vinca alkaloids and proteasome inhibitors. In some embodiments, the disease or condition is caused by or associated with axonal degeneration or neuronal cells damage.

A compound of Formula 1, 2, 3-1, 3-2, 3-3, 4, 5, 6-1, 6-2, 7-1, 7-2, 8-1, 8-2, 8-3, 8-4, 8-5, 8-6, 9-1, 9-2, 9-3, 9-4, 10-1, 10-2, 10-3, 10-4, 10-5, 10-6, 11-1, 11-2, 11-3, 11-4, 11-5, 11-6, 11-7, 12-1, 12-2, 12-3, 12-4, 12-5, 12-6, 12-7, or 12-8 (e.g., Compounds 1 to 468) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt may be administered, for example, various manners, such as orally, topically, rectally, parenterally, by inhalation spray, or via an implanted reservoir, although the most suitable route in any given case will depend on the particular host, and nature and severity of the conditions for which the active ingredient is being administered. The term “parenteral” as used herein includes subcutaneous, intracutaneous, intravenous, intramuscular, intraarticular, intraarterial, intrasynovial, intrasternal, intrathecal, intralesional and intracranial injection or infusion techniques. The compositions disclosed herein may be conveniently presented in unit dosage form and prepared by any of the methods well known in the art. Parenteral administration can be by continuous infusion over a selected period of time. Other forms of administration contemplated in this disclosure are as described in International Patent Application Nos. WO 2013/075083, WO 2013/075084, WO 2013/078320, WO 2013/120104, WO 2014/124418, WO 2014/151142, and WO 2015/023915.

The contacting is generally effected by administering to the subject an effective amount of one or more compounds, tautomers, solvates, stereoisomers, and pharmaceutically acceptable salt disclosed herein. Generally, administration is adjusted to achieve a therapeutic dosage of about 0.1 to 50 mg/kg, preferably 0.5 to 10 mg/kg, more preferably 1 to 10 mg/kg, though optimal dosages are compound specific, and generally empirically determined for each compound.

The dosage administered will be dependent on factors, such as the age, health and weight of the recipient, the extent of disease, type of concurrent treatment, if any, frequency of treatment, and the nature of the effect desired. In general, a daily dosage of the active ingredient can vary, for example, from 0.1 to 2000 milligrams per day. For example, 10-500 milligrams once or multiple times per day may be effective to obtain the desired results.

In some embodiments, 2 mg to 1500 mg or 5 mg to 1000 mg of a compound of Formula 1, 2, 3-1, 3-2, 3-3, 4, 5, 6-1, 6-2, 7-1, 7-2, 8-1, 8-2, 8-3, 8-4, 8-5, 8-6, 9-1, 9-2, 9-3, 9-4, 10-1, 10-2, 10-3, 10-4, 10-5, 10-6, 11-1, 11-2, 11-3, 11-4, 11-5, 11-6, 11-7, 12-1, 12-2, 12-3, 12-4, 12-5, 12-6, 12-7, or 12-8 (e.g., Compounds 1 to 468), disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt are administered once daily, twice daily, or three times daily. The compound, tautomer, solvate, stereoisomer, or pharmaceutically acceptable salt described herein is administered for morning/daytime dosing, with off period at night.

### A. EXAMPLES

In order that the disclosure described herein may be more fully understood, the following examples are disclosed herein. It should be understood that these examples are for illustrative purposes only and are not to be construed as limiting this disclosure in any way.

### Example I. Synthesis of Exemplary Compounds

The compounds of the disclosure, selected from a compound of the Formulae depicted herein, a tautomer thereof, a solvate or stereoisomer of the compound or the tautomer, or a pharmaceutically acceptable salt of the foregoing, can be made according to standard chemical practices or as illustrated herein, including the following general synthetic procedures and specific synthetic schemes for Compounds 1 to 468.

### Preparation of 2-(1-cyclopentylimidazol-2-yl)-5-methyl-1H-benzimidazole (2) Method 1

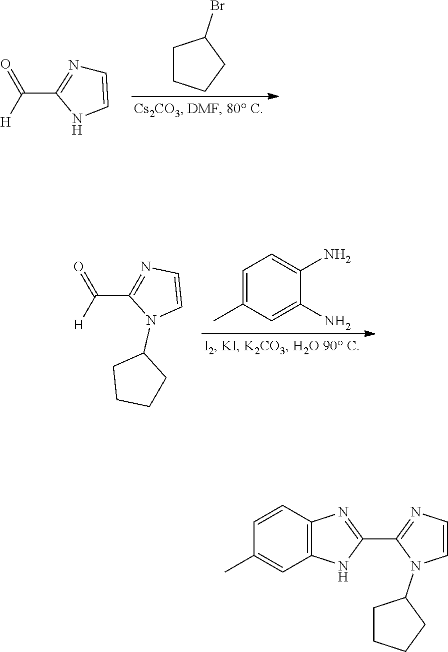

### Step 1. Preparation of 1-cyclopentyl-1H-imidazole-2-carbaldehyde

To a stirred solution of 1H-imidazole-2-carbaldehyde (1.00 eq, 1000 mg, 10.4 mmol) in DMF (5 mL), was added dicesium carbonate (2.00 eq, 6782 mg, 20.8 mmol). The resulted mixture was stirred for 30 min. After that, bromocyclopentane (1.50 eq, 1.7 mL, 15.6 mmol) was added dropwise, then the reaction solution was heated to 80° C., stirred for another 3 h. The reaction mixture was diluted with water and extracted with EtOAc extraction (3×20 ml), the organic layers were combined, washed with saturated brine and dried over anhydrous sodium sulfate, filtered and concentrate to obtain a crude product, which was purified by column chromatography to afford 1-cyclopentylimidazole-2-carbaldehyde (1700 mg, 10.4 mmol, 99.5% yield). MS (ESI) m/z 165 [M+H]⁺.

### Step 2: Preparation of 2-(1-cyclopentylimidazol-2-yl)-5-methyl-1H-benzimidazole

A solution of 1-cyclopentylimidazole-2-carbaldehyde (1.00 eq, 200 mg, 1.22 mmol) and 4-methylbenzene-1,2-diamine (67 mg, 0.552 mmol) in water (5 mL) was stirred at room temperature for 20 min, then K₂CO₃ (114 mg, 0.828 mmol) were added and the mixture stirred for another 10 min. KI (23 mg, 0.138 mmol) and 12 (1.00 eq, 140 mg, 0.552 mmol) was added, and the mixture was heated at 90° C. with stirring for 2 h. Sodium thiosulfate solution (10 mL; 5%) was added and the product was extracted with EtOAc (15 mL×3). The combined EtOAc layers were dried over anhydrous sodium sulfate and concentrated under reduced pressure to give a crude product, which was purified by with Prep-HPLC to give the product 2-(1-cyclopentylimidazol-2-yl)-5-methyl-1H-benzimidazole (178 mg, 0.668 mmol, 54.9% yield) as a white solid.

MS (ESI) m/z 267 [M+H]⁺.

¹H NMR (400 MHz, DMSO) δ 12.78 (br s, 1H), 7.59 (s, 1H), 7.48 (br s, 1H), 7.36 (br s, 1H), 7.18 (s, 1H), 7.03 (d, J=8.1 Hz, 1H), 6.14 (p, J=7.4 Hz, 1H), 2.42 (s, 3H), 2.26-2.16 (m, 2H), 1.92-1.65 (m, 6H).

Examples (Compounds) 3, 4, 6, 10, 33 were synthesized using a method similar to that used in Example 2.

| Example | | | | |
|---|---|---|---|---|
| No. | | | | MS |
| (Compound | | Appearance | | (m/z) |
| No.) | | and | | [M + |
| Method | Structure and Name | Yield | ¹H NMR Data | H]⁺ |
| Example 3 Method 1 | 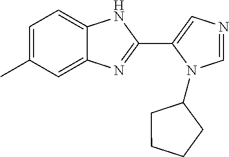 [SMILES: Cc1ccc2[nH]c(-c3cncn3C3CCCC3)nc2c1] | white solid, yield: 43.5% | ¹H NMR (400 MHz, DMSO) δ 12.58 (s, 1H), 8.06 (s, 1H), 7.65 (s, 1H), 7,23-7.58 (m, 2H), 7.01 (d, J = 9.3 Hz, 1H), 5.79 (p, J = 7.1 Hz, 1H), 2.42 (s, 3H), 2.25-2.14 (m, 2H), 1.90-1.63 (m, 6H). | 267 |
| | 2-(1-cyclopentyl-1H- | | | |
| | imidazol-5-yl)-6- | | | |
| | methyl-1H- | | | |
| | benzo[d]Imi-dazole | | | |
| Example 4 Method 1 | 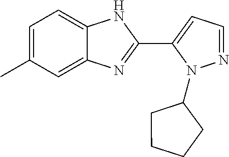 [SMILES: Cc1ccc2[nH]c(-c3ccnn3C3CCCC3)nc2c1] 2-(2-cyclopentylpyrazol-3- | white solid, yield: 19.9% | ¹H NMR (400 MHz, DMSO) δ 12.72 (s, 1H), 7.60 (d, J = 1.9 Hz, 1H), 7.51 (d, J = 8.1 Hz, 1H), 7.40 (s, 1H), 7.06 (d, J = 7.6 Hz, 1H), 6.93 (d, J = 1.9 Hz, 1H), 6.24-6.11 (m, 1H), 2.43 (s, 3H), 2.16-1.81 (m, 6H), 1.74-1.60 (m, 2H). | 267 |
| | yl)-5-methyl-1H-benzimidazole | | | |
| Example 6 Method 1 | 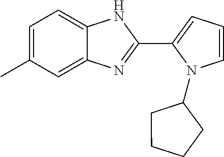 [SMILES: Cc1ccc2[nH]c(-c3cccn3C3CCCC3)nc2c1] 2-(1-cyclopentylpyrrol- 2-yl)-5-methyl-1H- benzimidazole | white solid, yield: 47.2% | ¹H NMR (400 MHz, DMSO) δ 12.24 (d, J = 10.4 Hz, 1H), 7.50- 7.35 (m, 1H), 7.34- 7.17 (m, 1H), 7.15 (t, J = 2.2 Hz, 1H), 6.96 (dd, J = 14.0, 8.2 Hz, 1H), 6.79 (dd, J = 3.8, 1.7 Hz, 1H), 6.19 (t, J = 3.3 DMSO) δ 12.24 (d, J = 10.4 Hz, 1H), 7.50- 7.35 (m, 1H), 7.34- 7.17 (m, 1H), 7.15 (t, J = 2.2 Hz, 1H), 6.96 (dd, | 266 |
| | | | J = 14.0, 8.2 Hz, 1H), | |
| | | | 6.79 (dd, J = 3.8, 1.7 | |
| | | | Hz, 1H), 6.19 (t, J = 3.3 | |
| | | | Hz, 1H), 6.07 (p, J = | |
| | | | 7.4 Hz, 1H), 2.40 (d, | |
| | | | J = 7.5 Hz, 3H), 2.20- | |
| | | | 2.05 (m, 2H), 1.91- | |
| | | | 1.55 (m, 6H). | |
| Example 10 Method 1 | 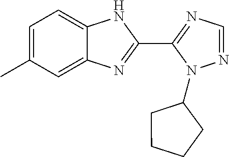 [SMILES: Cc1ccc2[nH]c(-c3ncnn3C3CCCC3)nc2c1] | white solid, yield: 27.6% | ¹H NMR (400 MHz, DMSO) δ 13.21 (s, 1H), 8.18 (s, 1H), 7.71- 7.51 (m, 1H), 7.50- 7.30 (m, 1H), 7.21- 7.02 (m, 1H), 6.33- 6.22 (m, 1H), 2.44 (s, 3H), 2.26-2.12 (m, 2H), 2.04-1.85 (m, 4H), 1.77-1.64 (m, 2H). | 268 |
| | 2-(2-cyclopentyl-1,2,4- | | | |
| | triazol-3-yl)-5-methyl- | | | |
| | 1H-benzimidazole | | | |
| Example 33 Method 1 | 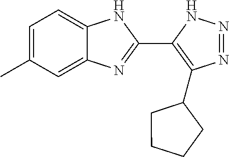 [SMILES: Cc1ccc2[nH]c(-c3[nH]nnc3C3CCCC3)nc2c1] | white solid, yield: 47.5% | ¹H NMR (400 MHz, DMSO-d₆) δ 15.40 (s, 0.5H), 14.97 (s, 0.5H), 12.76 (s, 1H), 7.56- 7.21 (m, 2H), 7.01 (d, J = 8.2 Hz, 1H), 4.00 (s, 1H), 2.42 (s, 3H), 2.19- 2.03 (m, 2H), 1.90-1.57 (d, J = 34.2 Hz, 6H). | 268 |
| | 2-(4-cyclopentyl-1H- | | | |
| | 1,2,3-triazol-5-yl)-5- | | | |
| | methyl-1H- | | | |
| | benzo[d]imidazole | | | |

### Preparation of 2-(3-cyclopentylpyridin-2-yl)-5-methyl-1H-benzo[d]imidazole (7) Method 2

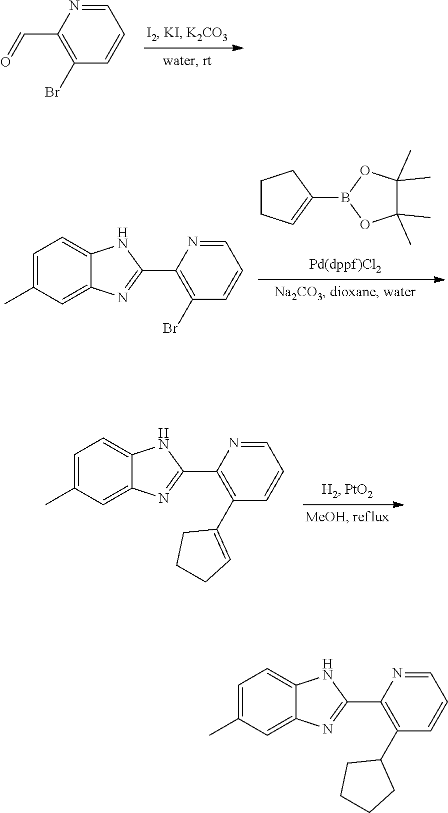

### Step 1. 2-(3-bromopyridin-2-yl)-5-methyl-1H-benzo[d]imidazole

A solution of 3-bromopyridine-2-carbaldehyde (500 mg, 2.69 mmol) and 4-methylbenzene-1,2-diamine (328 mg, 2.69 mmol) in water (10 mL) was stirred at room temperature for 20 min, then K₂CO₃ (556 mg, 4.03 mmol) was added and the mixture stirred for another 10 min. KI (112 mg, 0.67 mmol) and 12 (682 mg, 2.69 mmol) were added and the mixture was heated at 90° C. with stirring for 2 h. Sodium thiosulfate solution (10 mL) was added and the product was extracted with EtOAc (10 mL×3). The combined EtOAc layers were dried over anhydrous sodium sulfate and concentrated under reduced pressure to give a crude product, which was purified with flash chromatography (Silica gel column, 10 g, EtOAc/PE, 0 to 100%) to give the product 2-(3-bromopyridin-2-yl)-5-methyl-1H-benzo[d]imidazole (260 mg, 33.6% yield) as a yellow solid. MS (ESI) m/z 288 [M+H]⁺.

### Step 2. 2-(3-(cyclopent-1-en-1-yl)pyridin-2-yl)-5-methyl-1H-benzo[d]imidazole

To a solution 2-(3-bromo-2-pyridyl)-5-methyl-1H-benzimidazole (250 mg, 0.868 mmol), 2-(cyclopenten-1-yl)-4,4,5,5-tetramethyl-1,3,2-dioxaborolane (253 mg, 1.30 mmol) and Na₂CO₃ (184 mg, 1.74 mmol) in 1,4-Dioxane (10 mL) and water (2 mL), was added Pd(dppf)Cl₂ (63 mg, 0.0868 mmol) at room temperature under N₂. After addition, the mixture was stirred at 110° C. for 12 h. LCMS indicated the reaction was completed. Then the reaction mixture was added water (20 mL) and extracted with EtOAc (20 mL×3). The organics were then combined and dried (Na₂SO₄) before concentrating to dryness. The crude was purified by flash chromatography (Silica gel column, 10 g, EA/PE, 0 to 50%) to give the product 2-[3-(cyclopenten-1-yl)-2-pyridyl]-5-methyl-1H-benzimidazole (100 mg, 0.36 mmol, 41.8% yield) a yellow solid. MS (ESI) m/z 276 [M+H]⁺.

### Step 3. 2-(3-cyclopentylpyridin-2-yl)-5-methyl-1H-benzo[d]imidazole

To a solution of 2-[3-(cyclopenten-1-yl)-2-pyridyl]-5-methyl-1H-benzimidazole (100 mg, 0.363 mmol) in Methanol (5 mL), was added PtO₂ (30 mg) at room temperature under N2. After addition, the mixture was stirred at 75° C. for 12 h. LCMS indicated the reaction was completed. Then the reaction mixture was filtrated and concentrated to dryness. The crude was purified with prep-HPLC to give the product 2-(3-cyclopentylpyridin-2-yl)-5-methyl-1H-benzo[d]imidazole (0.98 mg, 1% yield) as a white solid. MS (ESI) m/z 278 [M+H]⁺.

¹H NMR (400 MHz, Methanol-d₄) δ 8.51 (s, 1H), 7.99 (d, J=8.0 Hz, 1H), 7.69-7.33 (m, 3H), 7.13 (d, J=7.2 Hz, 1H), 4.13-3.89 (m, 1H), 2.48 (s, 3H), 2.14-2.00 (m, 2H), 1.90-1.51 (m, 6H).

Examples (Compounds) 8, 9, 25 were synthesized using a method similar to that used in Example 7.

| Example | | | | |
|---|---|---|---|---|
| No. | | | | MS |
| (Compound | | Appearance | | (m/z) |
| No.) | | and | | [M + |
| Method | Structure and Name | Yield | ¹H NMR Data | H]⁺ |
| Example 8 Method 2 | 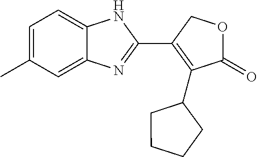 [SMILES: Cc1ccc2[nH]c(C3=C(C4CCCC4)C(=O)OC3)nc2c1] | white solid, yield: 2% | ¹H NMR (400 MHz, CDCl₃) δ 7.60 (d, J = 8.3 Hz, 1H), 7.46 (s, 1H), 7.19 (dd, J = 8.4, 1.5 Hz, 1H), 5.25 (s, 2H), 3.39 (p, J = 9.0 Hz, 1H), 2.51 (s, 3H), 2.17-2.03 (m, 2H), 2.03-1.87 (m, 3H), 1.80-1.63 (m, 3H). | 283 |
| | 3-Cyclopentyl-4-(5- | | | |
| | methyl-1H- | | | |
| | benzo[d]imidazol-2- | | | |
| | yl)furan-2(5H)-one | | | |
| Example 9 Method 2 | 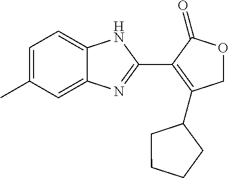 [SMILES: Cc1ccc2[nH]c(C3=C(C4CCCC4)COC3=O)nc2c1] | white solid, yield: 2% | ¹H NMR (400 MHz, CDCl₃) δ 11.04 (bs, 1H), 7.57 (bs, 1H), 7.44 (bs, 1H), 7.13 (dd, J = 8.2, 1.6 Hz, 1H), 5.02 (s, 2H), 4.57 (h, J = 8.1 Hz, 1H), 2.49 (s, 3H), 2.29 (dq, J = 13.3, 5.3 Hz, 2H), 1.97-1.35 (m, 6H). | 283 |
| | 4-Cyclopentyl-3-(5- | | | |
| | methyl-1H- | | | |
| | benzo[d]imidazol-2- | | | |
| | yl)furan-2(5H)-one | | | |
| Example 25 Method 2 | 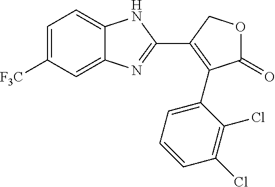 [SMILES: O=C1OCC(c2nc3cc(C(F)(F)F)ccc3[nH]2)=C1c1cccc(Cl)c1Cl] | white solid, yield: 3% | ¹H NMR (400 MHz, DMSO-d₆) δ 12.86 (s, 1H), 7.98 (s, 1H), 7.87- 7.73 (m, 2H), 7.57 (dd, J = 8.6, 1.7 Hz, 1H), 7.54-7.41 (m, 2H), 5.71-5.53 (m, 2H). | 413 |
| | 3-(2,3-Dichlorophenyl)- | | | |
| | 4-(5-(trifluoromethyl)- | | | |
| | 1H-benzo[d]imidazol-2- | | | |
| | yl)furan-2(5H)-one | | | |

### Preparation of [2-(4-cyclopentyl-1,2,4-triazol-3-yl)-5-methyl-1H-indol-3-yl]methanol (11)

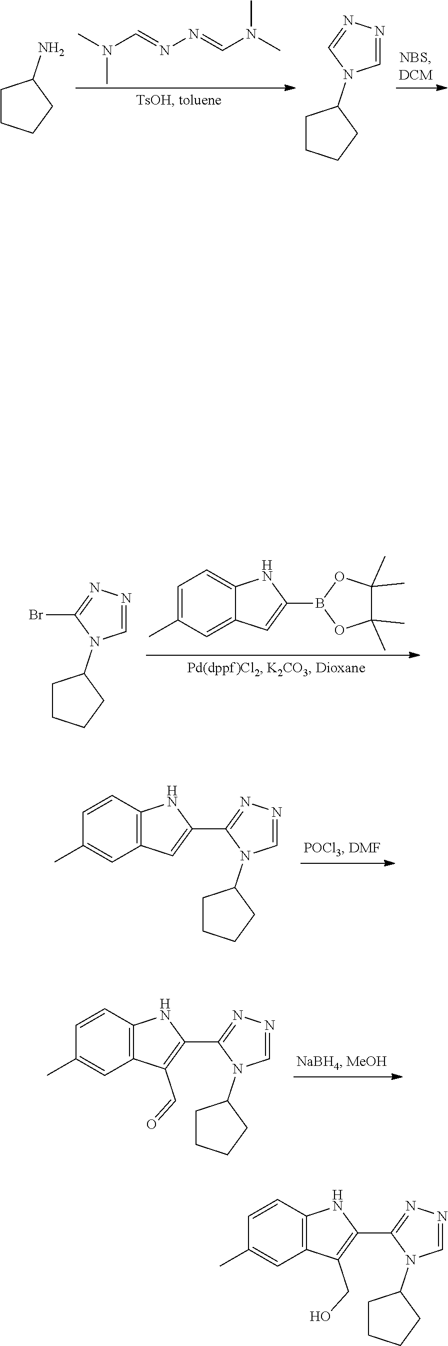

### Step 1. 4-cyclopentyl-4H-1,2,4-triazole

To a solution of cyclopentanamine (10.0 eq, 6.9 mL, 70.3 mmol) in Toluene (7 mL) was added N′-[(E)-dimethylaminomethyleneamino]-N,N-dimethyl-formamidine (1.00 eq, 1.00 g, 7.03 mmol) and 4-methylbenzenesulfonic acid (0.100 eq, 121 mg, 0.703 mmol). The mixture was heated to reflux and stirred for 24 h. The mixture was diluted with EtOAc and washed with saturated NaHCO₃. The organic layer was separated, dried over anhydrous sodium sulfate and concentrated under vacuum. The residue was purified by silica gel flash column chromatography (DCM/MeOH=3/97) to afford 4-cyclopentyl-1,2,4-triazole (542 mg, 3.95 mmol, 56.2% yield). MS (ESI) m/z 138 [M+H]⁺.

### Step 2. 3-bromo-4-cyclopentyl-4H-1,2,4-triazole

To a solution of 4-cyclopentyl-1,2,4-triazole (1.00 eq, 287 mg, 2.09 mmol) in DCM (10 mL) was added NBS (1.10 eq, 410 mg, 2.30 mmol). The mixture was stirred overnight at rt under dark. The mixture was concentrated and purified by silica gel flash column chromatography (DCM/MeOH=3/97) to afford 3-bromo-4-cyclopentyl-1,2,4-triazole (188 mg, 0.870 mmol, 41.6% yield). MS (ESI) m/z 216 [M+H]⁺.

### Step 3. 2-(4-cyclopentyl-4H-1,2,4-triazol-3-yl)-5-methyl-1H-indole

To a solution of 3-bromo-4-cyclopentyl-1,2,4-triazole (1.00 eq, 132 mg, 0.611 mmol) and 5-methyl-2-(4,4,5,5-tetramethyl-1,3,2-dioxaborolan-2-yl)-1H-indole (1.00 eq, 157 mg, 0.611 mmol) in water (0.6000 mL) and 1,4-Dioxane (3 mL) was added K₂CO₃ (3.00 eq, 253 mg, 1.83 mmol) and Pd(dppf)Cl₂ (0.100 eq, 45 mg, 0.0611 mmol). The mixture was heated to 90° C. and stirred overnight under N₂. The mixture was diluted with EtOAc and washed with brine. The organic layer was separated, dried over anhydrous sodium sulfate and concentrated under vacuum. The residue was purified by silica gel flash column chromatography (DCM/MeOH=3/97) to afford 2-(4-cyclopentyl-1,2,4-triazol-3-yl)-5-methyl-1H-indole (142 mg, 0.533 mmol, 87.3% yield). MS (ESI) m/z 267 [M+H]⁺.

### Step 4. 2-(4-cyclopentyl-4H-1,2,4-triazol-3-yl)-5-methyl-1H-indole-3-carbaldehyde

POCl₃ (3.00 eq, 0.040 mL, 0.428 mmol) was added into DMF (0.1100 mL) at 0° C. and stirred for 2 h. The mixture was added into a solution of 2-(4-cyclopentyl-1,2,4-triazol-3-yl)-5-methyl-1H-indole (1.00 eq, 38 mg, 0.143 mmol) in DCM (0.5000 mL) at 0° C. The reaction was quenched with saturated NaHCO₃ and extracted with EtOAc. The organic phase was separated, dried over anhydrous Na₂SO₄ and concentrated under vacuum. The residue was purified by silica gel flash column chromatography (DCM/MeOH=3/97) to afford 2-(4-cyclopentyl-1,2,4-triazol-3-yl)-5-methyl-1H-indole-3-carbaldehyde (19 mg, 0.0645 mmol, 45.2% yield). MS (ESI) m/z 295 [M+H]⁺.

### Step 5. (2-(4-cyclopentyl-4H-1,2,4-triazol-3-yl)-5-methyl-1H-indol-3-yl)methanol

To a solution of 2-(4-cyclopentyl-1,2,4-triazol-3-yl)-5-methyl-1H-indole-3-carbaldehyde (1.00 eq, 19 mg, 0.0645 mmol) in Methanol (1 mL) was added NaBH₄ (1.20 eq, 2.9 mg, 0.0775 mmol) at 0° C. The reaction mixture was stirred for 30 min. The reaction was quenched with saturated NH₄Cl. The organic phase was separated, dried over anhydrous Na₂SO₄ and concentrated under vacuum. The residue was purified by prep-HPLC to give [2-(4-cyclopentyl-1,2,4-triazol-3-yl)-5-methyl-1H-indol-3-yl]methanol (4.0 mg, 0.0135 mmol, 20.9% yield) as a white solid.

MS (ESI) m/z 297 [M+H]⁺.

¹H NMR (400 MHz, MeOD) δ 8.86 (s, 1H), 7.62 (dt, J=1.7, 0.9 Hz, 1H), 7.35 (d, J=8.3 Hz, 1H), 7.13 (dd, J=8.4, 1.6 Hz, 1H), 4.73 (s, 2H), 4.71-4.66 (m, 1H), 2.48 (s, 3H), 2.24-2.12 (m, 2H), 1.97-1.62 (m, 6H).

### Preparation of 5-cyclopentyl-4-(5-methyl-1H-benzo[d]imidazol-2-yl)-2,4-dihydro-3H-1,2,4-triazol-3-one (12) Method 3

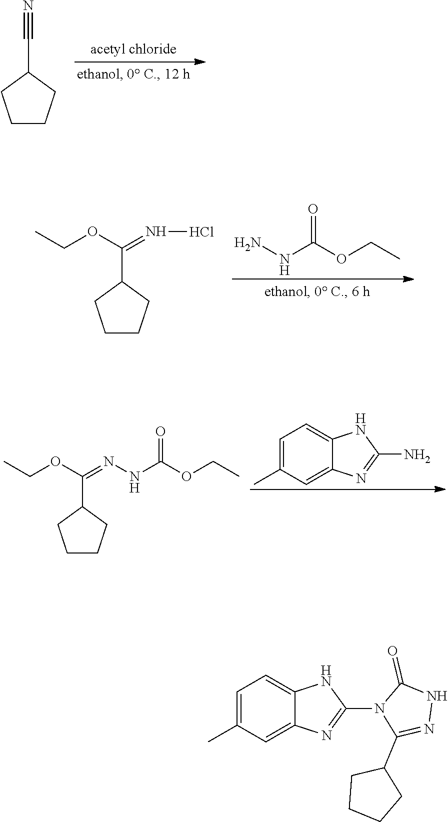

### Step 1. ethyl cyclopentanecarbimidate hydrochloride

cyclopentanecarbonitrile (5.00 g, 52.6 mmol) in ethanol (40 mL) was cooled to 0° C. by ice bath and treated with acetyl chloride (33.0 g, 420 mmol) dropwise over 0.5 hours. After complete addition, the reaction mixture was stirred at room temperature. After 16 h, the reaction mixture was concentrated under reduced pressure and the solid was triturated with diethyl ether and dried under vacuum to give ethyl cyclopentanecarbimidate hydrochloride (3 g, 16.9 mmol, 32% yield) as a white solid. MS (ESI) m/z 142 [M+H]f.

### Step 2. ethyl 2-(cyclopentyl(ethoxy)methylene)hydrazine-1-carboxylate

Ethyl cyclopentanecarbimidate hydrochloride (3 g, 16.9 mmol) in absolute ethanol (60 mL) was cooled using an ice bath. Ethyl ethyl hydrazinecarboxylate (1.75 g, 16.9 mmol) was added in absolute ethanol (60 mL) dropwise to the mixture. The reaction was stirred for 6 hours at 0° C. The solvent evaporated under reduced pressure. The residue was purified using flash chromatography (Silica gel column, 10 g, DCM/MeOH, 0 to 10%) to give ethyl 2-(cyclopentyl(ethoxy)methylene)hydrazine-1-carboxylate (3.3 g, 16.9 mmol, 85.6% yield) as a colorless oil. MS (ESI) m/z 229 [M+H]⁺.

### Step 3. 5-cyclopentyl-4-(5-methyl-1H-benzo[d]imidazol-2-yl)-2,4-dihydro-3H-1,2,4-triazol-3-one

A mixture of ethyl 2-(cyclopentyl(ethoxy)methylene)hydrazine-1-carboxylate (200 mg, 0.876 mmol) and 5-methyl-1H-benzimidazol-2-amine (129 mg, 0.876 mmol) was stirred at 165° C. for 2 h. Then the reaction mixture was cooled and diluted with MeOH and purified with Prep-HPLC to give the product 5-cyclopentyl-4-(5-methyl-1H-benzo[d]imidazol-2-yl)-2,4-dihydro-3H-1,2,4-triazol-3-one (1.6 mg, 0.0055 mmol, 0.63% yield) as a white solid. MS (ESI) m/z 284 [M+H]⁺.

¹H NMR (400 MHz, DMSO-d₆) δ 7.59-7.30 (m, 2H), 7.17 (d, J=8.4 Hz, 1H), 3.45-3.35 (m, 1H), 2.48 (s, 3H), 1.94-1.47 (m, 8H).

Examples (Compounds) 14 was synthesized using a method similar to that used in Example 12.

| Example | | | | |
|---|---|---|---|---|
| No. | | | | MS |
| (Compound | | Appearance | | (m/z) |
| No.) | | and | | [M + |
| Method | Structure and Name | Yield | ¹H NMR Data | H]⁺ |
| Example 14 Method 3 | 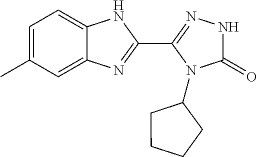 [SMILES: Cc1ccc2[nH]c(-c3n[nH]c(=O)n3C3CCCC3)nc2c1] | white solid, yield: 7.7% | ¹H NMR (400 MHz, DMSO-d₆) δ 13.04 (s, 1H), 12.26 (s, 1H), 7.75- 7.21 (m, 2H), 7.08 (s, 1H), 5.77 (s, 1H), 2.43 (s, 3H), 2.27-2.17 (m, 2H), 2.04-1.81 (m, 4H), 1.65-1.54 (m, 2H). | 284 |
| | 4-cyclopentyl-5-(5-methyl-1H- | | | |
| | benzo[d]imidazol-2-yl)- | | | |
| | 2,4-dihydro-3H-1,2,4-triazol-3-one | | | |

### Preparation of (1-Cyclopentyl-5-(5-methyl-1H-benzo[d]imidazol-2-yl)-1H-pyrrol-3-yl)methanol (16) Method 4

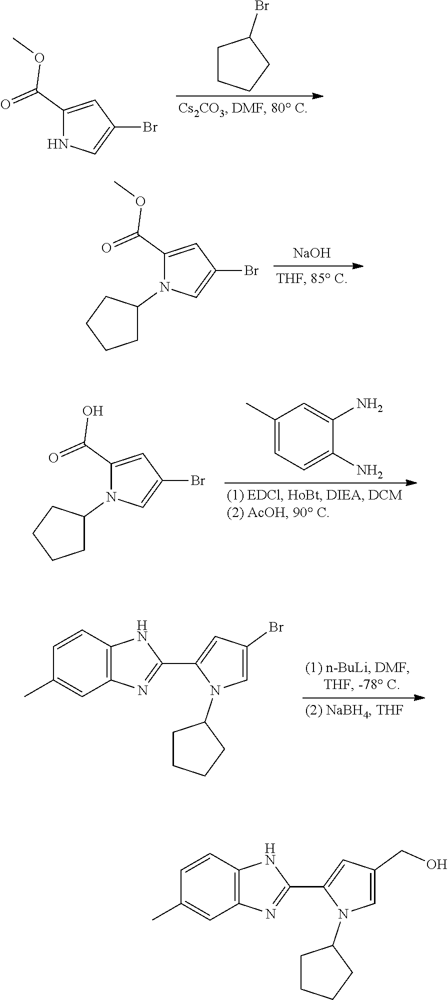

### Step 1. Methyl 4-bromo-1-cyclopentyl-1H-pyrrole-2-carboxylate

To a solution of methyl 4-bromo-1H-pyrrole-2-carboxylate (1.00 eq, 1000 mg, 4.90 mmol) in DMF (10 mL) was added cesium carbonate (2.00 eq, 3194 mg, 9.80 mmol) and the mixture was stirred for 30 min. Then bromocyclopentane (1.50 eq, 0.79 mL, 7.35 mmol) was added and the mixture was stirred for 24 h at 80° C. The reaction progress was monitored by TLC. Once completed, the mixture was diluted with DCM and washed with water. The organic layer was dried over Na₂SO₄, filtered and concentrated under reduced pressure. The residue was purified by column chromatography (PE/EA=3:1) to yield the product (760 mg, 57%).

### Step 2. 4-Bromo-1-cyclopentyl-1H-pyrrole-2-carboxylic acid

To a solution of methyl 4-bromo-1-cyclopentyl-pyrrole-2-carboxylate (1.00 eq, 760 mg, 2.79 mmol) in THF (10 mL) was added 15% NaOH solution (5 mL) and the mixture was stirred at 85° C. overnight. The reaction progress was monitored by LC/MS. Once completed, the mixture was acidified with 4 N HCl solution and extracted with EtOAc. The organic layer was dried over Na₂SO₄, filtered and concentrated under reduced pressure. The residue was used directly in the next step. MS (ESI) m/z 259 [M+H]⁺.

### Step 3. 2-(4-Bromo-1-cyclopentyl-1H-pyrrol-2-yl)-5-methyl-1H-benzo[d]imidazole

To a solution of 4-methylbenzene-1,2-diamine (1.00 eq, 201 mg, 1.65 mmol) in Toluene (5 mL) was added DIEA (1.90 eq, 0.54 mL, 3.13 mmol), HBTU (2.00 eq, 1249 mg, 3.29 mmol) and 4-bromo-1-cyclopentyl-pyrrole-2-carboxylic acid (1.00 eq, 425 mg, 1.65 mmol). Then the mixture was stirred at rt for 4 h. The reaction progress was monitored by LC/MS. Once completed, the solvent was removed under reduced pressure and the crude was dissolved in EtOAc and washed with water. The organic layer was dried and concentrated. The residue was dissolved in AcOH (2 mL) and the solution was stirred at 90° C. for 2 h. The reaction progress was monitored by LC/MS. Once completed, the solvent was removed and the residue was purified by column chromatography (PE/EA=1:1) to yield the product (270 mg, 48%). MS (ESI) m/z 345 [M+H]⁺.

### Step 4. (1-Cyclopentyl-5-(5-methyl-1H-benzo[d]imidazol-2-yl)-1H-pyrrol-3-yl)methanol

To a solution of 2-(4-bromo-1-cyclopentyl-pyrrol-2-yl)-5-methyl-1H-benzimidazole (1.00 eq, 65 mg, 0.189 mmol) in THF (5 mL) was added n-BuLi (2.00 eq, 24 mg, 0.378 mmol) dropwise at −78° C. and the mixture was stirred for 10 min at the same temperature. Then DMF (5.00 eq, 0.073 mL, 0.944 mmol) was added and the mixture was allowed to warm to rt and stirred for another 1 h. Then NaBH₄ (3.00 eq, 21 mg, 0.562 mmol) was added to the mixture at 0° C. and the mixture was stirred for 2 h at the same temperature. The reaction progress was monitored by LC/MS. Once completed, the solvent was removed under reduced pressure and the residue was purified by prep-HPLC to yield the product (3.5 mg, 6%). MS (ESI) m/z 295 [M+H]⁺.

¹H NMR (400 MHz, CDCl₃) δ 7.54 (s, 1H), 7.43 (s, 1H), 7.16-7.04 (m, 1H), 6.86 (s, 1H), 6.75 (s, 1H), 5.76 (s, 1H), 4.56 (s, 2H), 2.48 (s, 3H), 2.29-2.12 (m, 2H), 1.83-1.57 (m, 7H).

Examples (Compounds) 13, 17, 18, 19, 20, 24, 27, 28 were synthesized using a method similar to that used in Example 16.

| Example | | | | |
|---|---|---|---|---|
| No. | | | | MS |
| (Compound | | Appearance | | (m/z) |
| No.) | | and | | [M + |
| Method | Structure and Name | Yield | ¹H NMR Data | H]⁺ |
| Example 13 Method 4 | 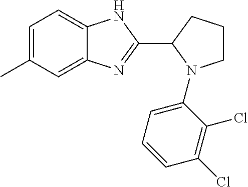 [SMILES: Cc1ccc2[nH]c(C3CCCN3c3cccc(Cl)c3Cl)nc2c1] | white solid, yield: 7% | ¹H NMR (400 MHz, DMSO-d₆) δ 7.41 (d, J = 8.3 Hz, 1H), 7.31 (s, 1H), 7.16-7.02 (m, 4H), 5.22 (t, J = 7.0 Hz, 1H), 4.11 (q, J = 7.8 Hz, 1H), 3.23 (m, 1H), 2.39 (s, 3H), 2.23- 2.05 (m, 2H), 2.00 (m, 2H). | 346 |
| | 2-(1-(2,3- | | | |
| | Dichlorophenyl)pyrrolidin- | | | |
| | 2-yl)-5-methyl-1H- | | | |
| | benzo[d]imidazole | | | |
| Example 17 Method 4 | 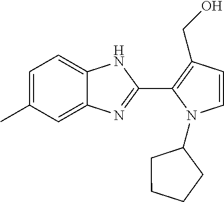 [SMILES: Cc1ccc2[nH]c(-c3c(CO)ccn3C3CCCC3)nc2c1] | white solid, yield: 10% | ¹H NMR (400 MHz, CDCl₃) δ 7.76-7.53 (m, 2H), 7.17 (d, J = 8.0 Hz, 1H), 6.98 (d, J = 2.5 Hz, 1H), 6.20 (d, J = 2.6 Hz, 1H), 5.79 (s, 1H), 4.60 (s, 2H), 2.49 (s, 3H), 2.32 (s, 2H), 1.72 (d, J = 6.5 Hz, 7H). | 295 |
| | (1-cyclopentyl-2-(5- | | | |
| | methyl-1H- | | | |
| | benzo[d]imidazol-2-yl)- | | | |
| | 1H-pyrrol-3- | | | |
| | yl)methanol | | | |
| Example 18 Method 4 | 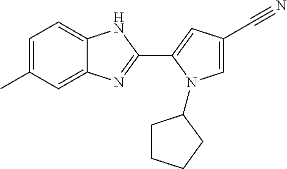 [SMILES: Cc1ccc2[nH]c(-c3cc(C#N)cn3C3CCCC3)nc2c1] | white solid, yield: 55% | ¹H NMR (400 MHz, CDCl₃) δ 7.56-7.48 (m, 1H), 7.43-7.36 (m, 2H), 7.74 (dd, J = 8.4 Hz, J = 1.2 Hz, 1H), 7.83 (d, J = 1.2 Hz, 1H), 5.92-5.82 (m, 1H), 2.47 (s, 3H), 2.31- 2.18 (m, 2H), 1.90- 1.66 (m, 6H). | 291 |
| | 1-cyclopentyl-5-(5- | | | |
| | methyl-1H- | | | |
| | benzo[d]imidazol-2-yl)- | | | |
| | 1H-pyrrole-3- | | | |
| | carbonitrile | | | |
| Example 19 Method 4 | 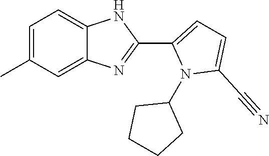 [SMILES: Cc1ccc2[nH]c(-c3ccc(C#N)n3C3CCCC3)nc2c1] 1-cyclopentyl-5-(5-methyl-1H-1,3- | white solid, yield: 54% | H NMR (400 MHz, DMSO-d₆) δ 7.51 (d, J = 7.6 Hz, 1H), 7.39 (s, 1H), 7.21 (d, J = 4.4 Hz, 1H), 7.07 (d, J = 8.4 Hz, 1H), 6.88 (d, J = 4.4 Hz, 1H), 6.52- 6.38 (m, 1H), 2.43 (s, 3H), 2.31-2.19 (m, 2H), 2.15-1.90 (m, 4H), 1.76-1.64 (m, 2H) | 291 |
| | benzodiazol-2-yl)pyrrole-2-carbonitrile | | | |
| Example 20 Method 4 | 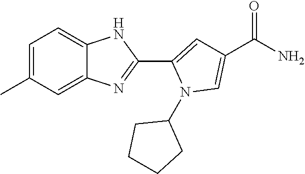 [SMILES: Cc1ccc2[nH]c(-c3cc(C(N)=O)cn3C3CCCC3)nc2c1] | white solid, yield: 57% | ¹H NMR (400 MHz, d4-MeOD) δ 7.66 (s, 1H), 7.37 (d, J = 8.0 Hz, 1 H), 7.28 (s, 1 H), 7.00 (d, J = 8.0 Hz, 1 H), 6.93 (s, 1 H), 5.58- 5.46 (m, 1H), 2.37 (s, 3H), 2.18-2.04 (m, 2H), 1.82-1.60 (m, 6H). | 309 |
| | 1-cyclopentyl-5-(5- | | | |
| | methyl-1H- | | | |
| | benzo[d]imidazol-2-yl)- | | | |
| | 1H-pyrrole-3- | | | |
| | carboxamide | | | |
| Example 24 Method 4 | 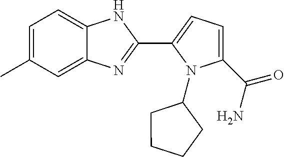 [SMILES: Cc1ccc2[nH]c(-c3ccc(C(N)=O)n3C3CCCC3)nc2c1] 1-cyclopentyl-5-(5-methyl-1H-1,3- | white solid, yield: 57% | ¹H NMR (400 MHz, CDCl₃) δ 7.54 (d, J = 8.0 Hz, 1H), 7.41 (s, 1H), 7.13 (m, J = 8.0 Hz, 1H), 6.64 (s, 1H), 6.44 (s, 1H), 5.90-5.55 (m, 3H), 2.49 (s, 3H), 2.35-2.20 (m, 2H), 2.12-1.98 (m, 2H), 1.95-1.80 (m, 2H), 1.65-1.47 (m, 2H). | 309 |
| | benzodiazol-2-yl)pyrrole-2-carboxamide | | | |
| Example 27 Method 4 | 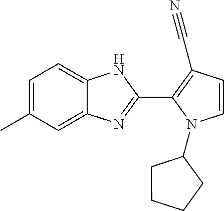 [SMILES: Cc1ccc2[nH]c(-c3c(C#N)ccn3C3CCCC3)nc2c1] | white solid, yield: 68% | ¹H NMR (400 MHz, CDCl₃) δ 9.82 (s, 1H), 7.52-7.48 (m, 2H), 7.14 (d, J = 8.4 Hz, 1H), 6.96 (d, J = 2.8 Hz, 1H), 6.56 (d, J = 2.8 Hz, 1H), 6.21-6.11 (m, 1H), 2.50 (s, 3H), 2.35-2.25 (m, 2H), 1.93-1.73 (m, 6H). | 291 |
| | 1-cyclopentyl-5-(5- | | | |
| | methyl-1H-1,3- | | | |
| | benzodiazol-2- | | | |
| | yl)pyrrole-2-carbonitrile | | | |
| Example 28 Method 4 | 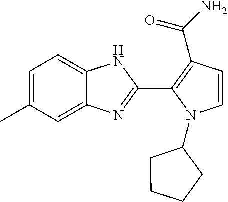 [SMILES: Cc1ccc2[nH]c(-c3c(C(N)=O)ccn3C3CCCC3)nc2c1] 1-cyclopentyl-5-(5- | white solid, yield: 30% | MS (ESI) m/z 309 [M + H]⁺. ¹H NMR (400 MHz, CDCl₃) δ 13.61 (bs, 1H), 7.65-7.35 (m, 2H), 7.08 (d, J = 8.4 Hz, 1H), 6.98 (d, J = 3.2 Hz, 1H), 6.57- 6.48 (m, 1H),6.46 (d, J = 3.2 Hz, 1H), 6.20- 5.80 (m, 1H), 5.70- 5.30 (m, 1H), 2.49 (s, 3H), 2.39- 2.25 (m, 2H), 1.92-1.72 (m, 6H) | 309 |
| | methyl-1H-1,3- | | | |
| | benzodiazol-2- | | | |
| | yl)pyrrole-2-carbonitrile | | | |

### Preparation of 5-(2,3-dichlorophenyl)-1-(5-methyl-1H-benzo[d]imidazol-2-yl)-1,2-dihydro-3H-1,2,4-triazol-3-one (26)

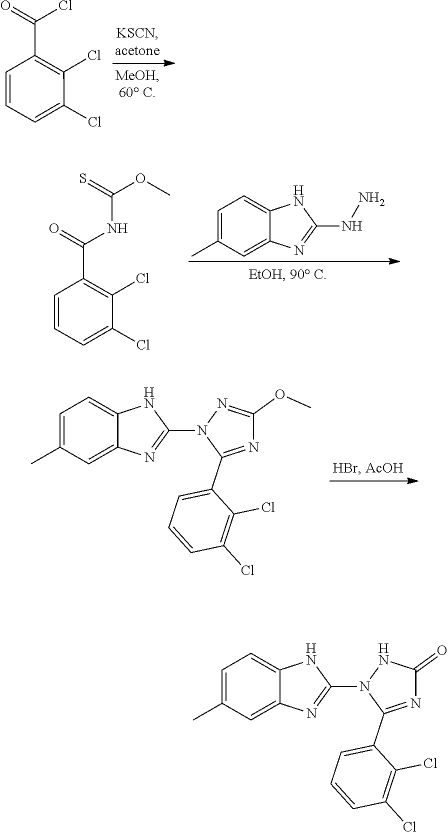

### Step 1. O-methyl (2,3-dichlorobenzoyl)carbamothioate

To a solution of 2,3-dichlorobenzoyl chloride (1.0 eq., 2000 mg, 9.55 mmol) in 30 mL acetone was added KSCN (1.0 eq., 928 mg, 9.55 mmol). The mixture was stirred at 60° C. for 3 h until the starting material was completely consumed. The reaction mixture was then cooled to room temperature and MeOH (2.5 eq., 765 mg, 23.9 mmol) was added to the reaction mixture. After stirring at 60° C. for another 8 h to afford O-methyl (2,3-dichlorobenzoyl)carbamothioate (1600 mg, 6.06 mmol, 63.400 yield). The crude product was directly used for the next step without further purification. MS (ESI) m/z 264/266 [M+H]⁺

### Step 2. 2-[5-(2,3-dichlorophenyl)-3-methoxy-1,2,4-triazol-1-yl]-5-methyl-1H-benzimidazole

The solution of (5-methyl-1H-benzimidazol-2-yl)hydrazine (1.00 eq, 246 mg, 1.51 mmol) and O-methyl N-(2,3-dichlorobenzoyl)carbamothioate (1.00 eq, 400 mg, 1.51 mmol) in methanol (6 mL) was stirred at 80° C. for 48 h. The solvent was removed under reduced pressure and the residue was purified by silica gel column chromatography to afford 2-[5-(2,3-dichlorophenyl)-3-methoxy-1,2,4-triazol-1-yl]-5-methyl-1H-benzimidazole (130 mg, 0.347 mmol, 22.9% yield).

MS (ESI) m/z 374/376 [M+H]⁺.

### Step 3. 3-(2,3-dichlorophenyl)-2-(5-methyl-1H-benzimidazol-2-yl)-1H-1,2,4-triazol-5-one

2-[5-(2,3-dichlorophenyl)-3-methoxy-1,2,4-triazol-1-yl]-5-methyl-1H-benzimidazole (1.00 eq, 200 mg, 0.534 mmol) was dissolved in a solution of hydrogen bromide (10.0 eq, 432 mg, 5.34 mmol) in Acetic acid (1 mL), the resulted solution was heated up to 100° C., and stirred for 6 h. The reaction mixture was quenched with sat. NaHCO₃ (aq), then extracted with EtOAc. All the organic layers were combined and dried over anhydrous Na₂SO₄ and concentrated in vacuo. The residue was purified by pre-HPLC to afford 3-(2,3-dichlorophenyl)-2-(5-methyl-1H-benzimidazol-2-yl)-1H-1,2,4-triazol-5-one (5.0 mg, 0.0139 mmol, 2.6% yield).

MS (ESI) m/z 360/362 [M+H]⁺.

¹H NMR (400 MHz, DMSO) δ 12.99 (s, 1H), 12.12 (s, 1H), 7.87-7.82 (m, 1H), 7.65 (dd, J=7.7, 1.5 Hz, 1H), 7.52 (t, J=7.9 Hz, 1H), 7.33-7.24 (m, 1H), 7.20 (d, J=27.5 Hz, 1H), 6.98 (dd, J=31.9, 8.1 Hz, 1H), 2.36 (d, J=23.6 Hz, 3H).

### Preparation of 3-(2,3-dichlorophenyl)-4-(5-methyl-1H-benzo[d]imidazol-2-yl)-1,2,4-oxadiazol-5 (4H)-one (29) Method 5

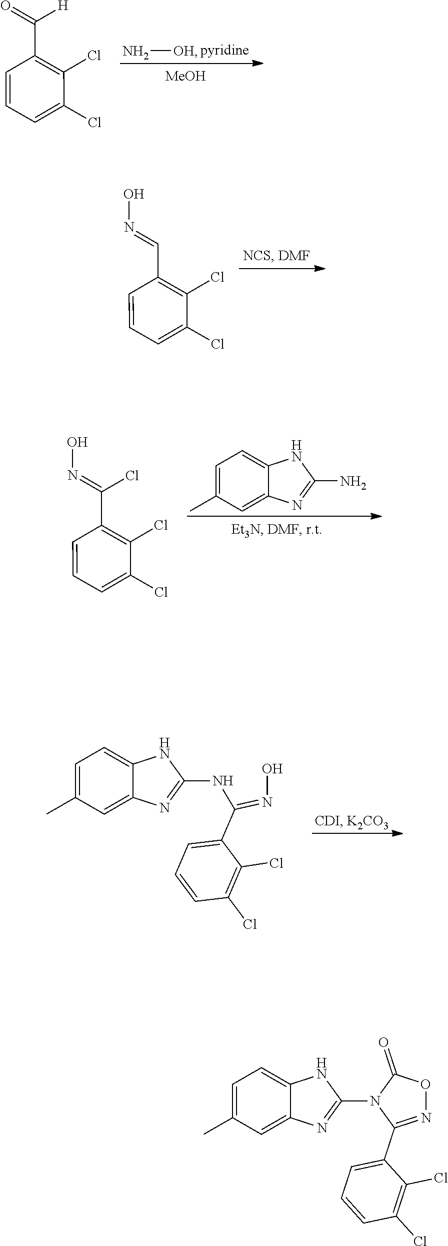

### Step 1. (1E)-2,3-dichlorobenzaldehyde oxime

Hydroxylammonium Chloride (1.20 eq, 476 mg, 6.86 mmol) was added to a solution of the 2,3-dichlorobenzaldehyde (1.00 eq, 1000 mg, 5.71 mmol) in methanol (30 mL), followed by pyridine (1.00 eq, 0.46 mL, 5.71 mmol). The reaction mixture was stirred at r.t. for 2.5 h. Then the solvent was removed in vacuo, the residue was suspended in DCM (120 ml) and washed with 1 M HCl solution (3×30 ml), H₂O (3×30 ml) and brine solution. The organic layer was dried (Na₂SO₄) and then concentrated at reduced pressure. The residue was purified by silica gel column chromatography eluting with hexane-EtOAc or recrystallization to afford (1E)-2,3-dichlorobenzaldehyde oxime (630 mg, 3.32 mmol, 58.0% yield). MS (ESI) m/z 190/192 [M+H]⁺.

### Step 2. (1Z)-2,3-dichloro-N-hydroxy-benzimidoyl chloride

To a stirred solution of (1E)-2,3-dichlorobenzaldehyde oxime (1.00 eq, 200 mg, 1.05 mmol) in DMF (5 mL) was added m-CPBA (5.00 eq, 703 mg, 5.26 mmol), then stirred at 20° C. overnight. The reaction mixture was quenched with sat. aq. NaHCO₃ and Na₂S₂O₃, then extracted with EtOAc. All the organic layers were combined and concentrated at reduced pressure. The residue was purified to afford (1Z)-2,3-dichloro-N-hydroxy-benzimidoyl chloride (120 mg, 0.535 mmol, 50.79%% yield). MS (ESI) m/z 224/226 [M+H]⁺.

### Step 3. 2,3-dichloro-N′-hydroxy-N-(5-methyl-1H-benzimidazol-2-yl)benzamidine

To a stirred solution of (1Z)-2,3-dichloro-N-hydroxy-benzimidoyl chloride (1.00 eq, 120 mg, 0.535 mmol) and 5-methyl-1H-benzimidazol-2-amine (1.00 eq, 79 mg, 0.535 mmol) in DMF (1 mL) was added triethylamine (2.00 eq, 108 mg, 1.07 mmol). The reaction mixture was stirred r.t. for overnight. The solvent was removed under reduced pressure and the residue was purified by silica gel column to afford 2,3-dichloro-N′-hydroxy-N-(5-methyl-1H-benzimidazol-2-yl)benzamidine (155 mg, 0.462 mmol, 86.5% yield). MS (ESI) m/z 335/337 [M+H]⁺.

### Step 4. 3-(2,3-dichlorophenyl)-4-(5-methyl-1H-benzimidazol-2-yl)-1,2,4-oxadiazol-5-one

To a solution of 2,3-dichloro-N′-hydroxy-N-(5-methyl-1H-benzimidazol-2-yl)benzamidine (1.00 eq, 155 mg, 0.462 mmol) in MeCN (4 mL) was added CDI (1.20 eq, 90 mg, 0.555 mmol), followed by K₂CO₃ (5.00 eq, 320 mg, 2.31 mmol). The resulting mixture was stirred at r.t. for 20 min. The crude mixture was concentrated under reduced pressure and then purified by short column chromatography using EtOAc in hexane to give the corresponding product 3-(2,3-dichlorophenyl)-4-(5-methyl-1H-benzimidazol-2-yl)-1,2,4-oxadiazol-5-one (23 mg, 0.0637 mmol, 13.8% yield)

MS (ESI) m/z 361/363 [M+H]⁺.

¹H NMR (400 MHz, DMSO) δ 13.01 (br s, 1H), 7.85-7.75 (m, 2H), 7.62-7.51 (m, 2H), 7.37-7.10 (m, 2H), 2.42 (d, J=4.1 Hz, 3H).

Examples (Compounds) 32 was synthesized using a method similar to that used in Example 29.

| Example | | | | |
|---|---|---|---|---|
| No. | | | | MS |
| (Compound | | Appearance | | (m/z) |
| No.) | | and | | [M + |
| Method | Structure and Name | Yield | ¹H NMR Data | H]⁺ |
| Example 32 Method 5 | 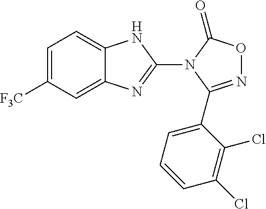 [SMILES: O=c1onc(-c2cccc(Cl)c2Cl)n1-c1nc2cc(C(F)(F)F)ccc2[nH]1] | white solid, yield: 4.3% | ¹H NMR (400 MHz, CDCl₃) δ 8.48-8.19 (m, 1H), 7.96-7.76 (m, 1H), 7.74-7.66 (m, 2H), 7.50-7.37 (m, 2H). | 415/417 |
| | 3-(2,3-dichlorophenyl)- | | | |
| | 4-(5-methyl-1H- | | | |
| | benzo[d]imidazol-2-yl)- | | | |
| | 1,2,4-oxadiazol-5(4H)- | | | |
| | one | | | |

### Preparation of 4-(2,3-dichlorophenyl)-5-(5-(trifluoromethyl)-1H-benzo[d]imidazol-2-yl)-2,4-dihydro-3H-1,2,4-triazol-3-one (36)

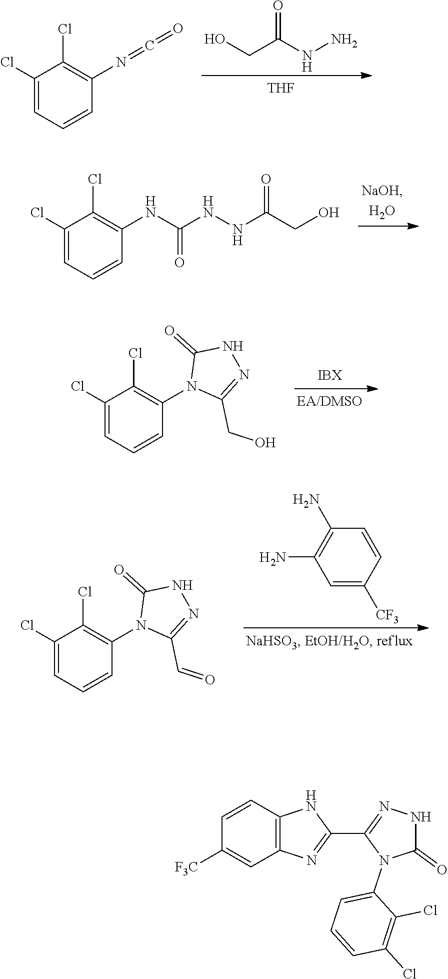

### Step 1. N-(2,3-dichlorophenyl)-2-(2-hydroxyacetyl)hydrazine-1-carboxamide

To a solution of 1,2-dichloro-3-isocyanatobenzene (4 g, 21.3 mmol) in THF (100 mL) was added 2-hydroxyacetohydrazide (1.86 g, 20.6 mmol) under N₂. The mixture was stirred at rt for 2 hours under N₂. The mixture was filtered and concentrated under vacuum to give N-(2,3-dichlorophenyl)-2-(2-hydroxyacetyl)hydrazine-1-carboxamide (5.05 g, 86%) as a white solid.

MS (ESI) m/z 278 [M+H]⁺.

### Step 2. 4-(2,3-dichlorophenyl)-5-(hydroxymethyl)-2,4-dihydro-3H-1,2,4-triazol-3-one

To a solution of N-(2,3-dichlorophenyl)-2-(2-hydroxyacetyl)hydrazine-1-carboxamide (50 mg, 0.18 mmol) in H₂O (0.5 mL) was added NaOH aq. (0.18 mL, 0.18 mmol, 1 mol/L in H₂O) and stirred at 105° C. for 3 hours. The mixture was concentrated. The residue was purified by reverse HPLC to give 4-(2,3-dichlorophenyl)-5-(hydroxymethyl)-2,4-dihydro-3H-1,2,4-triazol-3-one (41.5 mg, 11%) as a white solid. MS (ESI) m/z 260 [M+H]⁺.

### Step 3. 4-(2,3-dichlorophenyl)-5-oxo-4,5-dihydro-1H-1,2,4-triazole-3-carbaldehyde

To a solution of 4-(2,3-dichlorophenyl)-5-(hydroxymethyl)-2,4-dihydro-3H-1,2,4-triazol-3-one (100 mg, 0.39 mmol) in EA/DMSO (10 mL/1.5 mL) was added IBX (540 mg, 1.93 mmol). The mixture was refluxed for 1 hour. To the mixture was added water (5 mL) and extracted with EtOAc (5 mL×2). The combined organic layers were dried over anhydrous Na₂SO₄ and concentrated. The residue was purified by reverse HPLC to give 4-(2,3-dichlorophenyl)-5-oxo-4,5-dihydro-1H-1,2,4-triazole-3-carbaldehyde (91 mg, 92%) as a white solid. MS (ESI) m/z 258 [M+H]⁺.

### Step 4. 4-(2,3-dichlorophenyl)-5-(5-(trifluoromethyl)-1H-benzo[d]imidazol-2-yl)-2,4-dihydro-3H-1,2,4-triazol-3-one

To a solution of 4-(2,3-dichlorophenyl)-5-oxo-4,5-dihydro-1H-1,2,4-triazole-3-carbaldehyde (91 mg, 0.35 mmol) in EtOH/H₂O (20 mL/5.3 mL) was added NaHSO₃ (73 mg, 0.7 mmol). The mixture was stirred at 0° C. for 1 hour. 4-(trifluoromethyl)benzene-1,2-diamine (62 mg, 0.35 mmol) was added to the mixture. The mixture was stirred at 80° C. for 16 hours. The mixture was concentrated, water (5 mL) was added and extracted with EtOAc (5 mL×2). The combined organic layers were dried over anhydrous Na₂SO₄ and concentrated. The residue was purified by prep-HPLC to give 4-(2,3-dichlorophenyl)-5-(5-(trifluoromethyl)-1H-benzo[d]imidazol-2-yl)-2,4-dihydro-3H-1,2,4-triazol-3-one (39.3 mg, 27%) as a white solid.

MS (ESI) m/z 414 [M+H]⁺.

¹H NMR (400 MHz, DMSO-d₆) δ 13.11 (s, 2H), 7.86 (dd, J=8.0, 1.2 Hz, 1H), 7.78 (s, 1H), 7.72-7.63 (m, 2H), 7.58-7.51 (m, 2H).

### Preparation of 2-(3-(2,3-dichlorophenyl)-5-oxo-1,5-dihydro-4H-1,2,4-triazol-4-yl)-5-(trifluoromethyl)-1H-indole-3-carbonitrile (41) Method 6

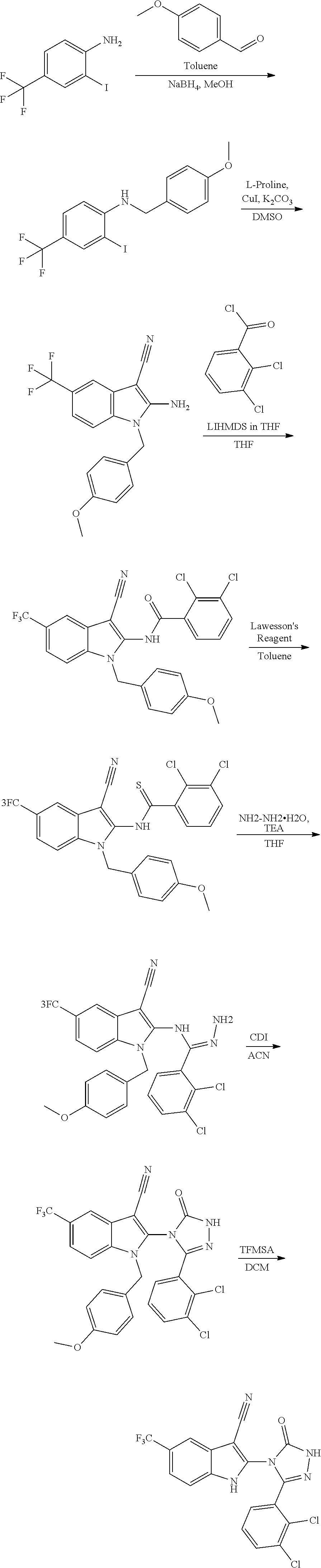

### Step 1. 2-iodo-N-(4-methoxybenzyl)-4-(trifluoromethyl)aniline

To a solution of 2-iodo-4-(trifluoromethyl)aniline (1.00 eq, 1430 mg, 4.98 mmol), 4-methoxybenzaldehyde (1.00 eq, 678 mg, 4.98 mmol) in Toluene (15 mL) was added one drop of AcOH. The mixture was stirred at 100° C. for 16 h. Then the mixture was concentrated, the residue was added Methanol (15 mL). NaBH₄ (2.00 eq, 2112 mg, 9.96 mmol) was then added. The mixture was stirred at r.t. for 1 h. 20 mL of water was added to the reaction mixture, the reaction mixture was extracted with EtOAc. The combined organic layer was washed with brine, dried over MgSO₄, and concentrated under vacuum. The residue was subjected to flash chromatography (20% EtOAc in hexanes) to give 2-iodo-N-[(4-methoxyphenyl)methyl]-4-(trifluoromethyl)aniline (1.20 g, 2.95 mmol, 59.15% yield) as a yellow solid. MS (ESI) m/z 406.1 [M+H]⁺.

### Step 2. 2-amino-1-(4-methoxybenzyl)-5-(trifluoromethyl)-1H-indole-3-carbonitrile

To a solution of 2-iodo-N-[(4-methoxyphenyl)methyl]-4-(trifluoromethyl)aniline (1.00 eq, 700 mg, 1.72 mmol) in DMSO (50 mL) propanedinitrile (1.20 eq, 136 mg, 2.06 mmol), L-Proline (0.200 eq, 40 mg, 0.344 mmol), CuI (0.100 eq, 33 mg, 0.172 mmol), K₂CO₃ (2.00 eq, 475 mg, 3.44 mmol) were added. The reaction mixture was stirred at 60° C. for 16 h under an argon atmosphere. 100 mL of water was added to the reaction mixture, the reaction mixture was extracted with EtOAc. The combined organic layers were washed with brine, dried over MgSO₄, and concentrated under vacuum. The residue was purified by a SiO₂ gel column, eluting with EtOAc in hexane from 0-40% to afford 2-amino-1-[(4-methoxyphenyl)methyl]-5-(trifluoromethyl)indole-3-carbonitrile (350 mg, 0.963 mmol, 56.01% yield) as brown oil. MS (ESI) m/z 346.1 [M+H]⁺.

### Step 3. 2,3-dichloro-N-[3-cyano-1-[(4-methoxyphenyl)methyl]-5-(trifluoromethyl)indol-2-yl]benzamide

To a stirred solution of 2-amino-1-[(4-methoxyphenyl)methyl]-5-(trifluoromethyl)indole-3-carbonitrile (1.00 eq, 350 mg, 1.01 mmol) in THF (15 mL) was added LiHMDS in THF (1.0 M) (2.00 eq, 2.0 mL, 2.03 mmol) dropwise at −10° C. The resulting mixture was stirred for 30 min. Then 2,3-dichlorobenzoyl chloride (2.00 eq, 425 mg, 2.03 mmol) in THF (5 mL) was added dropwise. The resulting mixture was stirred for 1 h. The reaction was quenched with NaHCO₃ (aq). The resulting mixture was extracted with DCM. The combined organic layers were washed with brine, dried over anhydrous Na₂SO₄. After filtration, the filtrate was concentrated under reduced pressure. The residue was purified by flash chromatography (C18) to afford 2,3-dichloro-N-[3-cyano-1-[(4-methoxyphenyl)methyl]-5-(trifluoromethyl)indol-2-yl]benzamide (402 mg, 0.776 mmol, 76.52% yield) as a white solid. MS (ESI) m/z 516.1 [M+H]⁺

### Step 4. 2,3-dichloro-N-[3-cyano-1-[(4-methoxyphenyl)methyl]-5-(trifluoromethyl)indol-2-yl]benzenecarbothioamide

To a solution of 2,3-dichloro-N-[3-cyano-1-[(4-methoxyphenyl)methyl]-5-(trifluoromethyl)indol-2-yl]benzamide (1.00 eq, 150 mg, 0.289 mmol) in Toluene (5 mL) was added Lawesson's Reagent (2.00 eq, 234 mg, 0.579 mmol). Then the solution was stirred at 60° C. for 4 h. After cooling to r.t., 20 mL of water was added. The mixture was extracted with DCM (20 mL×3), the organic layer were combined and washed with water, dried over Na₂SO₄ and concentrated. The residue was purified by flash chromatography (C18) to afford 2,3-dichloro-N-[3-cyano-1-[(4-methoxyphenyl)methyl]-5-(trifluoromethyl)indol-2-yl]benzenecarbothioamide (100 mg, 0.187 mmol, 64.66% yield) as a yellow solid. (EST) m/z 534.2 [M+H]⁺.

### Step 5. N′-amino-2,3-dichloro-N-[3-cyano-1-[(4-methoxyphenyl)methyl]-5-(trifluoromethyl)indol-2-yl]benzamidine

To a solution of TEA (2.00 eq, 0.052 mL, 0.374 mmol) in THF (10 mL) were added 2,3-dichloro-N-[3-cyano-1-[(4-methoxyphenyl)methyl]-5-(trifluoromethyl)indol-2-yl]benzenecarbothioamide (1.00 eq, 100 mg, 0.187 mmol) and hydrazine hydrate (1.00 eq, 12 mg, 0.187 mmol). Then the mixture was stirred at 80° C. for 1 h. After cooling to r.t. 20 mL of water was added. The mixture was extracted with DCM (20 mL×3), the organic layer were combined and washed with water, dried over Na₂SO₄ and concentrated. The residue was purified by flash chromatography (C18) to afford N′-amino-2,3-dichloro-N-[3-cyano-1-[(4-methoxyphenyl)methyl]-5-(trifluoromethyl)indol-2-yl]benzamidine (60 mg, 0.113 mmol, 60.23% yield) as a yellow solid. (ESI) m/z 532.1 [M+H]⁺.

### Step 6. 2-[3-(2,3-dichlorophenyl)-5-oxo-1H-1,2,4-triazol-4-yl]-1-[(4-methoxyphenyl)methyl]-5-(trifluoromethyl)indole-3-carbonitrile

To a solution of N′-amino-2,3-dichloro-N-[3-cyano-1-[(4-methoxyphenyl)methyl]-5-(trifluoromethyl)indol-2-yl]benzamidine (1.00 eq, 20 mg, 0.0376 mmol) in MeCN (10 mL) was added di(imidazol-1-yl)methanone (3.00 eq, 18 mg, 0.113 mmol). The reaction mixture was stirred at 80° C. for 2 h. After cooling to r.t., 10 mL of water was added to the mixture. The mixture was extracted with DCM (10 mL×3), the organic layers were combined and washed with water, dried over Na₂SO₄ and evaporated. The residue was purified by flash chromatography (C18) to afford 2-[3-(2,3-dichlorophenyl)-5-oxo-1H-1,2,4-triazol-4-yl]-1-[(4-methoxyphenyl)methyl]-5-(trifluoromethyl)indole-3-carbonitrile (15 mg, 0.0269 mmol, 71.51% yield) as a yellow solid. (ESI) m/z 558.2 [M+H]⁺.

### Step 7. 2-(3-(2,3-dichlorophenyl)-5-oxo-1,5-dihydro-4H-1,2,4-triazol-4-yl)-5-(trifluoromethyl)-1H-indole-3-carbonitrile

To a solution of 2-[3-(2,3-dichlorophenyl)-5-oxo-1H-1,2,4-triazol-4-yl]-1-[(4-methoxyphenyl)methyl]-5-(trifluoromethyl)indole-3-carbonitrile (1.00 eq, 12 mg, 0.0215 mmol) in DCM (2 mL) was added TFA (1.0 mL) at 0° C. The solution was stirred at 0° C. for 2 h. The pH of the solution was adjusted to 9 with Sat. K₂CO₃ (aq). The resulting solution was extracted with DCM (10 mL×3). The organic layers were combined, washed with water (15 mL), dried and concentrated under vacuum. The residue was purified by prep-TLC (MeOH/DCM=1/10) to afford 2-[3-(2,3-dichlorophenyl)-5-oxo-1H-1,2,4-triazol-4-yl]-5-(trifluoromethyl)-1H-indole-3-carbonitrile (5.0 mg, 0.0114 mmol, 53.09% yield) as a white solid.

(ESI) m/z 438.2 [M+H]⁺.

¹H NMR (400 MHz, Methanol-d₄) δ 8.41 (s, 1H), 7.79-7.73 (m, 2H), 7.70 (dd, J=8.6, 1.9 Hz, 1H), 7.58 (dd, J=7.7, 1.6 Hz, 1H), 7.46 (t, J=7.9 Hz, 1H).

Examples (Compounds) 30, 31 were synthesized using a method similar to that used in Example 41.

| Example | | | | |
|---|---|---|---|---|
| No. | | | | MS |
| (Compound | | Appearance | | (m/z) |
| No.) | | and | | [M + |
| Method | Structure and Name | Yield | ¹H NMR Data | H]⁺ |
| Example 30 Method 6 | 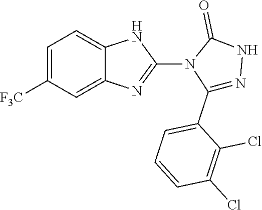 [SMILES: O=c1[nH]nc(-c2cccc(Cl)c2Cl)n1-c1nc2cc(C(F)(F)F)ccc2[nH]1] | white solid, yield: 7% | ¹H NMR (400 MHz, DMSO-d6) δ 8.10 (s, 1H), 7.86 (ddd, J = 21.9, 7.9, 1.6 Hz, 2H), 7.63-7.50 (m, 2H), 7.30 (d, J = 8.2 Hz, 1H) | 414 |
| | 3-(2,3-Dichlorophenyl)- | | | |
| | 4-[5-(trifluoromethyl)- | | | |
| | 1H-benzimidazol-2-yl]- | | | |
| | 1H-1,2,4-triazol-5-one | | | |
| Example 31 Method 6 | 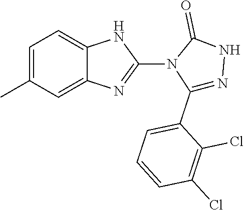 [SMILES: Cc1ccc2[nH]c(-n3c(-c4cccc(Cl)c4Cl)n[nH]c3=O)nc2c1] | white solid, yield: 18% | ¹H NMR (400 MHz, DMSO-d6) δ 8.10 (s, 1H), 7.86 (ddd, J = 21.9, 7.9, 1.6 Hz, 2H), 7.63-7.50 (m, 2H), 7.30 (d, J = 8.2 Hz, 1H), 2.49 (s, 3H) | 360 |
| | 5-(2,3-dichlorophenyl)- | | | |
| | 4-(5-methyl-1H- | | | |
| | benzo[d]imidazol-2-yl)- | | | |
| | 2,4-dihydro-3H-1,2,4- | | | |
| | triazol-3-one | | | |

### Preparation of 5-bromo-2-(4-(2,3-dichlorophenyl)-4H-1,2,4-triazol-3-yl)-1H-benzo[d]imidazole (49) Method 7

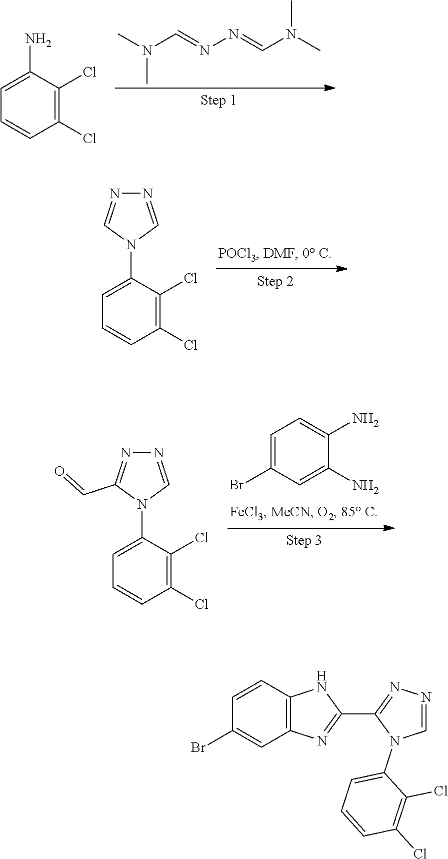

### Step 1. Preparation of 4-(2,3-dichlorophenyl)-4H-1,2,4-triazole

The mixture of 2,3-dichloroaniline (10 g, 0.0617 mol), (E)-N′-((E)-(dimethylamino)methylene)-N,N-dimethylformohydrazonamide (8.77 g, 0.0617 mol) was stirred for 20 hours at 260° C. After cooling, the reaction mixture was diluted with water (60 mL), and extracted with EtOAc (20 mL×3). All the organic layers were combined, dried over anhydrous Na₂SO₄ and concentrated under reduced pressure to give crude product that was purified through column chromatography on silica gel (CH₃OH/DCM=1:10) to give the 4-(2,3-dichlorophenyl)-4H-1,2,4-triazole (11.5 g, 87.03% yield) as a white solid. MS (ESI) m/z 214.1 [M+H]⁺.

### Step 2. Preparation of 4-(2,3-dichlorophenyl)-4H-1,2,4-triazole-3-carbaldehyde

POCl₃ (11 mL) was dissolved in DMF (32 mL) and the resulting solution was cooled to 0° C. under a N₂ atmosphere for 2 h. 4-(2,3-dichlorophenyl)-4H-1,2,4-triazole (5 g, 0.0234 mol) dissolved in DCM (42 mL) was added to the above system by syringe and stirred at 0° C. for 16 h. After the reaction was completed, the reaction mixture was diluted with water (60 mL), extracted EtOAc (20 mL×3). All the Organic layers were combined, dried over anhydrous Na₂SO₄ and concentrated under reduced pressure to give crude product that was purified through column chromatography on silica gel (EA/PE=1:1) to give the 4-(2,3-dichlorophenyl)-4H-1,2,4-triazole-3-carbaldehyde (4.5 g, 79.49% yield) as a white solid. MS (ESI) m/z 242.1 [M+H]⁺.

### Step 3. Preparation of 5-bromo-2-(4-(2,3-dichlorophenyl)-4H-1,2,4-triazol-3-yl)-1H-benzo[d]imidazole

A mixture solution of 4-(2,3-dichlorophenyl)-4H-1,2,4-triazole-3-carbaldehyde (4.5 g, 0.0186 mol), 4-bromobenzene-1,2-diamine (3.48 mg, 0.0186 mol) and FeCl₃·H₂O (1.01 g, 0.0037 mol) was stirred at 85° C. for 16 h. Then the second batch of FeCl₃·H₂O (1.01 g, 0.0037 mol) was added. The reaction mixture was stirred at 85° C. for 2 h under O₂. After cooling, the reaction mixture was diluted with water (60 mL), extracted with EtOAc (20 mL×3). All the organic layers were combined, dried over anhydrous Na₂SO₄ and concentrated under reduced pressure to give crude product that was purified through column chromatography on silica gel (EA:PE=1:3) to give the 5-bromo-2-(4-(2,3-dichlorophenyl)-4H-1,2,4-triazol-3-yl)-1H-benzo[d]imidazole (2.5 g, 32.8% yield) as a red white solid.

MS (ESI) m/z 410 [M+H]⁺.

¹H NMR (400 MHz, DMSO-d6) δ 13.79 (s, 1H), 9.05 (s, 1H), 7.92 (dd, J=8.2, 1.5 Hz, 1H), 7.78 (dd, J=8.0, 1.5 Hz, 1H), 7.66 (d, J=1.9 Hz, 1H), 7.59 (t, J=8.1 Hz, 1H), 7.45 (d, J=8.6 Hz, 1H), 7.34 (dd, J=8.7, 1.9 Hz, 1H).

Examples (Compounds) 1, 5, 15, 46, 50, 72, 93, 94, 95, 96, 109, 128, 132 were synthesized using a method similar to that used in Example 49.

| Example | | | | |
|---|---|---|---|---|
| No. | | | | MS |
| (Compound | | Appearance | | (m/z) |
| No.) | | and | | [M + |
| Method | Structure and Name | Yield | ¹H NMR Data | H]⁺ |
| Example 1 Method 7 | 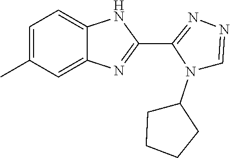 [SMILES: Cc1ccc2[nH]c(-c3nncn3C3CCCC3)nc2c1] | white solid, yield: 5.6% | ¹H NMR (400 MHz, CDCl₃) δ 8.39 (s, 1H), 7.68 (d, J = 8.3 Hz, 1H), 7.56 (s, 1H), 7.16 (dd, J = 8.4, 1.6 Hz, 1H), 6.19-6.05 (m, 1H), 2.51 (s, 3H), 2.50- 2.41 (m, 2H), 1.98- 1.84 (m, 6H). | 268 |
| | 2-(4-cyclopentyl-4H- | | | |
| | 1,2,4-triazol-3-yl)-5- | | | |
| | methyl-1H- | | | |
| | benzo[d]imidazole | | | |
| Example 5 Method 7 | 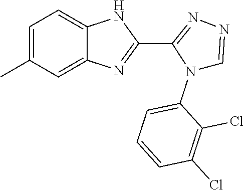 [SMILES: Cc1ccc2[nH]c(-c3nncn3-c3cccc(Cl)c3Cl)nc2c1] | white solid, yield: 5% | ¹H NMR (400 MHz, CDCl₃) δ 8.36 (s, 1H), 7.73 (dd, J = 8.0, 1.6 Hz, 1H), 7.58-7.42 (m, 4H), 7.16-7.09 (m, 1H), 2.46 (s, 3H). | 344 |
| | 2-(4-(2,3- | | | |
| | dichlorophenyl)-4H- | | | |
| | 1,2,4-triazol-3-yl)-5- | | | |
| | methyl-1H- | | | |
| | benzo[d]imidazole | | | |
| Example 15 Method 7 | 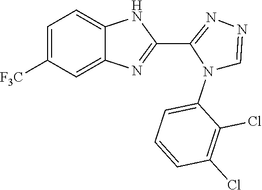 [SMILES: FC(F)(F)c1ccc2[nH]c(-c3nncn3-c3cccc(Cl)c3Cl)nc2c1] | white solid, yield: 18.2% | ¹H NMR (400 MHz, DMSO) δ 14.08 (d, J = 10.5 Hz, 1H), 9.10 (d, J = 2.3 Hz, 1H), 7.94 (dd, J = 8.2, 1.5 Hz, 1H), 7.83-7.44 (m, 5H). | 398/400 |
| | 2-[4-(2,3- | | | |
| | dichlorophenyl)-1,2,4- | | | |
| | triazol-3-yl]-5- | | | |
| | (trifluoromethyl)-1H- | | | |
| | benzimidazole | | | |
| Example 46 Method 7 | 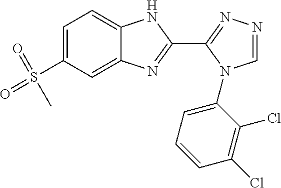 [SMILES: CS(=O)(=O)c1ccc2[nH]c(-c3nncn3-c3cccc(Cl)c3Cl)nc2c1] | white solid, yield: 6.2% | ¹H NMR (400 MHz, DMSO-d₆) δ 9.11 (s, 1H), 8.01 (s, 1H), 7.93 (dd, J = 8.2, 1.5 Hz, 1H), 7.80 (dd, J = 8.0, 1.5 Hz, 1H), 7.78-7.69 (m, 2H), 7.61 (t, J = 8.1 Hz, 1H), 3.20 (s, 3H). | 408/410 |
| | 2-(4-(2,3- | | | |
| | dichlorophenyl)-4H- | | | |
| | 1,2,4-triazol-3-yl)-5- | | | |
| | (methylsulfonyl)-1H- | | | |
| | benzo[d]imidazole | | | |
| Example 50 Method 7 | 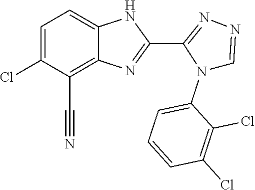 [SMILES: N#Cc1c(Cl)ccc2[nH]c(-c3nncn3-c3cccc(Cl)c3Cl)nc12] | white solid, yield: 0.3% | 1H NMR (400 MHz, DMSO-d6) δ 8.84 (s, 1H), 7.83 (dd, J = 8.2, 1.5 Hz, 1H), 7.62 (d, J = 7.7 Hz, 1H), 7.64-7.47 (m, 2H), 7.07 (d, J = 8.6 Hz, 1H). | 390 |
| | 5-chloro-2-(4-(2,3- | | | |
| | dichlorophenyl)-4H- | | | |
| | 1,2,4-triazol-3-yl)-1H- | | | |
| | benzo[d]imidazole-4- | | | |
| | carbonitrile | | | |
| Example 72 Method 7 | 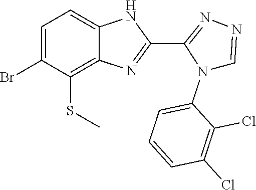 [SMILES: CSc1c(Br)ccc2[nH]c(-c3nncn3-c3cccc(Cl)c3Cl)nc12] | white solid, yield: 32.3% | ¹H NMR (400 MHz, DMSO) δ 13.91 (s, 1H), 9.05 (s, 1H), 7.90 (dd, J = 8.2, 1.5 Hz, 1H), 7.79 (dd, J = 7.9, 1.5 Hz, 1H), 7.60 (t, J = 8.1 Hz, 1H), 7.43 (d, J = 8.6 Hz, 1H), 7.28 (d, J = 8.6 Hz, 1H), 2.23 (s, 3H). | 454/456 |
| | 5-bromo-2-(4-(2,3- | | | |
| | dichlorophenyl)-4H- | | | |
| | 1,2,4-triazol-3-yl)-4- | | | |
| | (methylthio)-1H- | | | |
| | benzo[d]imidazole | | | |
| Example 93 Method 7 | 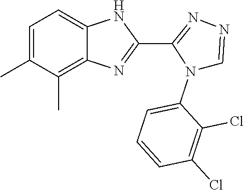 [SMILES: Cc1ccc2[nH]c(-c3nncn3-c3cccc(Cl)c3Cl)nc2c1C] | yellow solid, yield: 11.4% | ¹H NMR (400 MHz, DMSO-d₆) δ 13.34 (s, 1H), 9.03 (d, J = 5.8 Hz, 1H), 7.95-7.88 (m, 1H), 7.77 (d, J = 8.0 Hz, 1H), 7.59 (t, J = 8.0 Hz, 1H), 7.17 (m, 1H), 7.00 (m, 1H), 2.48 (s, 1H), 2.31 (s, 1H), 2.24 (s, 2H), 2.09 (s, 2H). | 358 |
| | 2-(4-(2,3- | | | |
| | dichlorophenyl)-4H- | | | |
| | 1,2,4-triazol-3-yl)-4,5- | | | |
| | dimethyl-1H- | | | |
| | benzo[d]imidazole | | | |
| Example 94 Method 7 | 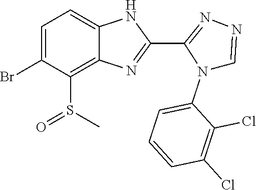 [SMILES: CS(=O)c1c(Br)ccc2[nH]c(-c3nncn3-c3cccc(Cl)c3Cl)nc12] | white solid, yield: 22% | ¹H NMR (400 MHz, DMSO) δ 13.69 (s, 1H), 9.09 (s, 1H), 7.90 (dd, J = 8.2, 1.5 Hz, 1H), 7.79 (s, 1H), 7.66-7.46 (m, 3H), 2.87 (s, 3H). | 470/472 |
| | 5-bromo-2-(4-(2,3- | | | |
| | dichlorophenyl)-4H- | | | |
| | 1,2,4-triazol-3-yl)-4- | | | |
| | (methylsulfinyl)-1H- | | | |
| | benzo[d]imidazole | | | |
| Example 95 Method 7 | 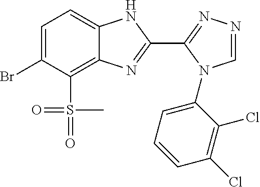 [SMILES: CS(=O)(=O)c1c(Br)ccc2[nH]c(-c3nncn3-c3cccc(Cl)c3Cl)nc12] | white solid, yield: 25.5% | ¹H NMR (400 MHz, DMSO) δ 14.22 (s, 1H), 9.10 (s, 1H), 7.91 (dd, J = 8.2, 1.5 Hz, 1H), 7.78 (dd, J = 8.0, 1.5 Hz, 1H), 7.70 (d, J = 8.6 Hz, 1H), 7.66-7.55 (m, 2H), 3.07 (s, 3H). | 486/488 |
| | 5-bromo-2-(4-(2,3- | | | |
| | dichlorophenyl)-4H- | | | |
| | 1,2,4-triazol-3-yl)-4- | | | |
| | (methylsulfonyl)-1H- | | | |
| | benzo[d]imidazole | | | |
| Example 96 Method 7 | 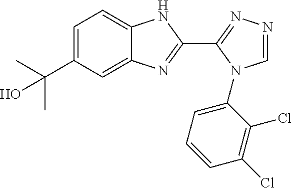 [SMILES: CC(C)(O)c1ccc2[nH]c(-c3nncn3-c3cccc(Cl)c3Cl)nc2c1] | white solid, yield: 21.1% | 1H NMR (400 MHz, DMSO-d₆) δ 13.42 (d, J = 9.5 Hz, 1H), 9.01 (s, 1H), 7.91 (dd, J = 8.3, 2.7 Hz, 1H), 7.77 (dd, J = 8.0, 1.5 Hz, 1H), 7.66-7.55 (m, 2H), 7.49-7.40 (m, 1H), 7.37-7.19 (m, 1H), 5.00 (d, J = 47.0 Hz, 1H), 1.44 (d, J = 10.5 Hz, 6H). | 389 |
| | 2-(2-(4-(2,3- | | | |
| | dichlorophenyl)-4H- | | | |
| | 1,2,4-triazol-3-yl)-1H- | | | |
| | benzo[d]imidazol-5- | | | |
| | yl)propan-2-ol | | | |
| Example 109 Method 7 | 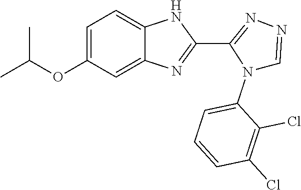 [SMILES: CC(C)Oc1ccc2[nH]c(-c3nncn3-c3cccc(Cl)c3Cl)nc2c1] | white solid, yield: 14.9% | 1H NMR (400 MHz, DMSO-d6) δ 13.29 (s, 1H), 8.99 (s, 1H), 7.90 (dd, J = 8.3, 1.5 Hz, 1H), 7.76 (dd, J = 8.0, 1.5 Hz, 1H), 7.59Z (t, J = 8.1 Hz, 1H), 7.41- 7.27 (m, 1H), 6.94 (d, J = 2.3 Hz, 1H), 6.86- 6.70 (m, 1H), 4.64- 4.49 (m, 1H), 1.26 (d, J = 5.9 Hz, 6H). | 388 |
| | 2-(4-(2,3- | | | |
| | dichlorophenyl)-4H- | | | |
| | 1,2,4-triazol-3-yl)-5- | | | |
| | isopropoxy-1H- | | | |
| | benzo[d]imidazole | | | |
| Example 128 Method 7 | 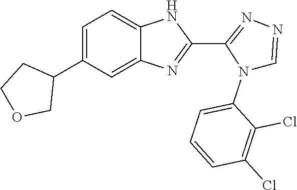 [SMILES: Clc1cccc(-n2cnnc2-c2nc3cc(C4CCOC4)ccc3[nH]2)c1Cl] | yellow solid, yield: 5.2% | ¹H NMR (400 MHz, DMSO-d₆) δ 13.46 (s, 1H), 8.99 (s, 1H), 7.87 (dd, J = 7.8, 3.6 Hz, 1H), 7.74 (d, J = 7.2 Hz, 1H), 7.55 (m, 1H), 7.42-7.27 (m, 2H), 7.10 (dd, J = 49.2, 8.4 Hz, 1H), 4.02-3.87 (m, 2H), 3.73 (m, 1H), 3.55-3.35 (m, 2H), 2.27 (m, 1H), 1.87 (m, 1H). | 400 |
| | 2-(4-(2,3- | | | |
| | dichlorophenyl)-4H- | | | |
| | 1,2,4-triazol-3-yl)-5- | | | |
| | (tetrahydrofuran-3-yl)- | | | |
| | 1H-benzo[d]imidazole | | | |
| Example 132 Method 7 | 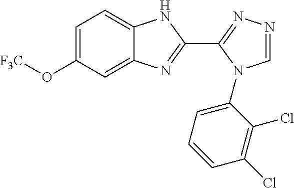 [SMILES: FC(F)(F)Oc1ccc2[nH]c(-c3nncn3-c3cccc(Cl)c3Cl)nc2c1] | white solid, yield: 6% | ¹H NMR (400 MHz, DMSO) δ 13.86 (s, 1H), 9.06 (s, 1H), 7.92 (dd, J = 8.2, 1.5 Hz, 1H), 7.79 (dd, J = 8.0, 1.5 Hz, 1H), 7.63-7.55 (m, J = 8.1 Hz, 2H), 7.46 (s, 1H), 7.28-7.14 (m, 1H). | 414/416 |
| | 2-(4-(2,3- | | | |
| | dichlorophenyl)-4H- | | | |
| | 1,2,4-triazol-3-yl)-5- | | | |
| | (trifluoromethoxy)-1H- | | | |
| | benzo[d]imidazole | | | |

### Preparation of 2-(4-(2,3-dichlorophenyl)-4H-1,2,4-triazol-3-yl)-5-(3-methyl-1H-pyrazol-5-yl)-1H-benzo[d]imidazole (51) Method 8

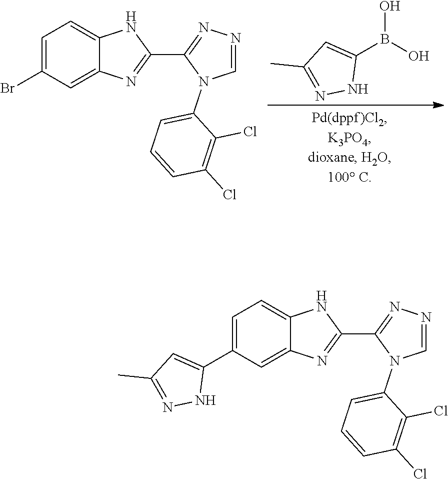

To a solution of 5-bromo-2-[4-(2,3-dichlorophenyl)-1,2,4-triazol-3-yl]-1H-benzimidazole (1.00 eq, 155 mg, 0.379 mmol) in 1,4-Dioxane (5 mL) and water (1 mL), was added (3-methyl-1H-pyrazol-5-yl)boronic acid (2.00 eq, 95 mg, 0.758 mmol), Pd(dppf)Cl₂ (0.300 eq, 92 mg, 0.114 mmol) and K₃PO₄ (3.00 eq, 241 mg, 1.14 mmol). The mixture was stirred at 100° C. for 2 h. The mixture was diluted with EtOAc and washed with water. The organic layers were combined, dried over anhydrous sodium sulfate and purified by prep-HPLC to give 2-[4-(2,3-dichlorophenyl)-1,2,4-triazol-3-yl]-5-(3-methyl-1H-pyrazol-5-yl)-1H-benzimidazole (13 mg, 0.0301 mmol, 7.9% yield).

MS (ESI) m/z 410/412 [M+H]⁺.

¹H NMR (400 MHz, DMSO) δ 13.56 (s, 1H), 12.52 (s, 1H), 9.03 (s, 1H), 7.95-7.75 (m, 3H), 7.64-7.43 (m, 3H), 6.43 (s, 1H), 2.23 (s, 3H).

Examples (Compounds) 55, 56, 68, 73, 74, 78, 79, 81 were synthesized using a method similar to that used in Example 51.

| Example | | | | |
|---|---|---|---|---|
| No. | | | | MS |
| (Compound | | Appearance | | (m/z) |
| No.) | | and | | [M + |
| Method | Structure and Name | Yield | ¹H NMR Data | H]⁺ |
| Example 55 Method 8 | 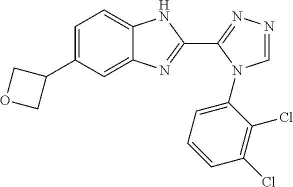 [SMILES: Clc1cccc(-n2cnnc2-c2nc3cc(C4COC4)ccc3[nH]2)c1Cl] | white solid, yield: 8% | ¹H NMR (400 MHz, DMSO-d₆) δ 13.53 (s, 1H), 9.02 (d, J = 1.5 Hz, 1H), 7.92 (dd, J = 8.2, 1.5 Hz, 1H), 7.77 (dd, J = 8.0, 1.5 Hz, 1H), 7.60 (t, J = 8.1 Hz, 1H), 7.43 (d, J = 24.4 Hz, 3H), 6.62 (s, 1H), 6.39-6.29 (m, 1H), 4.81 (s, 1H), 4.11 (s, 2H). | 387 |
| | 2-(4-(2,3- | | | |
| | dichlorophenyl)-4H- | | | |
| | 1,2,4-triazol-3-yl)-5- | | | |
| | (oxetan-3-yl)-1H- | | | |
| | benzo[d]imidazole | | | |
| Example 56 Method 8 | 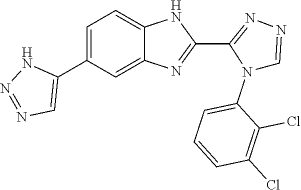 [SMILES: Clc1cccc(-n2cnnc2-c2nc3cc(-c4cnn[nH]4)ccc3[nH]2)c1Cl] | white solid, yield: 3.3% | ¹H NMR (400 MHz, DMSO-d₆) δ 9.06 (s, 1H), 8.36 (s, 1H), 8.01- 7.91 (m, 2H), 7.80 (dd, J = 8.0, 1.4 Hz, 2H), 7.61 (t, J = 8.2 Hz, 1H), 7.55 (d, J = 8.2 Hz, 1H). | 397 |
| | 2-(4-(2,3- | | | |
| | dichlorophenyl)-4H- | | | |
| | 1,2,4-triazol-3-yl)-5- | | | |
| | (1H-1,2,3-triazol-5-yl)- | | | |
| | 1H-benzo[d]imidazole | | | |
| Example 68 Method 8 | 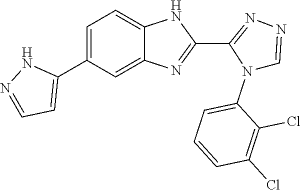 [SMILES: Clc1cccc(-n2cnnc2-c2nc3cc(-c4ccn[nH]4)ccc3[nH]2)c1Cl] | white solid, yield: 8.8% | ¹H NMR (400 MHz, DMSO-d₆) δ 9.05 (s, 1H), 7.94 (dd, J = 8.2, 1.4 Hz, 1H), 7.89 (s, 1H), 7.80 (dd, J = 8.0, 1.4 Hz, 1H), 7.69 (s, 2H), 7.61 (t, J = 8.1 Hz, 1H), 7.50 (d, J = 8.4 Hz, 1H), 6.73 (d, J = 2.2 Hz, 1H). | 396 |
| | 2-(4-(2,3- | | | |
| | dichlorophenyl)-4H- | | | |
| | 1,2,4-triazol-3-yl)-5- | | | |
| | (1H-pyrazol-5-yl)-1H- | | | |
| | benzo[d]imidazole | | | |
| Example 73 Method 8 | 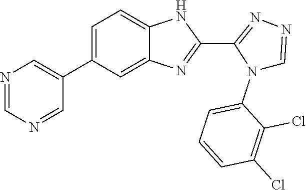 [SMILES: Clc1cccc(-n2cnnc2-c2nc3cc(-c4cncnc4)ccc3[nH]2)c1Cl] | white solid, yield: 3.8% | ¹H NMR (400 MHz, DMSO-d₆) δ 13.78 (s, 1H), 9.18 (s, 3H), 9.06 (s, 1H), 7.97-7.90 (m, 2H), 7.81 (dd, J = 8.0, 1.5 Hz, 1H), 7.63 (dt, J = 16.2, 5.7 Hz, 3H) | 409 |
| | 2-(4-(2,3- | | | |
| | dichlorophenyl)-4H- | | | |
| | 1,2,4-triazol-3-yl)-5- | | | |
| | (pyrimidin-5-yl)-1H- | | | |
| | benzo[d]imidazole | | | |
| Example 74 Method 8 | 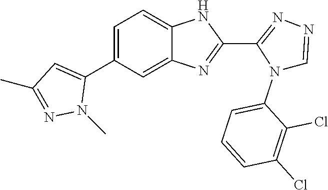 [SMILES: Cc1cc(-c2ccc3[nH]c(-c4nncn4-c4cccc(Cl)c4Cl)nc3c2)n(C)n1] | white solid, yield: 21.7% | 1H NMR (400 MHz, DMSO-d₆) δ 13.71 (s, 1H), 9.05 (s, 1H), 7.92 (dd, J = 8.2, 1.5 Hz, 1H), 7.79 (dd, J = 7.9, 1.5 Hz, 1H), 7.65-7.51 (m, 3H), 7.32 (s, 1 H), 6.15 (s, 1H), 3.73 (s, 3H), 2.16 (s, 3H). | 425 |
| | 2-(4-(2,3- | | | |
| | dichlorophenyl)-4H- | | | |
| | 1,2,4-triazol-3-yl)-5- | | | |
| | (1,3-dimethyl-1H- | | | |
| | pyrazol-5-yl)-1H- | | | |
| | benzo[d]imidazole | | | |
| Example 78 Method 8 | 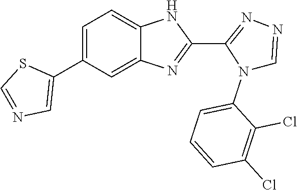 [SMILES: Clc1cccc(-n2cnnc2-c2nc3cc(-c4cncs4)ccc3[nH]2)c1Cl] | white solid, yield: 4.8% | ¹H NMR (400 MHz, DMSO-d₆) δ 13.73 (s, 1H), 9.03 (s, 1H), 8.27 (s, 1H), 7.90 (dd, J = 8.2, 1.2 Hz, 1H), 7.77 (dd, J = 8.0, 1.4 Hz, 2H), 7.58 (t, J = 8.2 Hz, 4H) | 413 |
| | 5-(2-(4-(2,3- | | | |
| | dichlorophenyl)-4H- | | | |
| | 1,2,4-triazol-3-yl)-1H- | | | |
| | benzo[d]imidazol-5- | | | |
| | yl)thiazole | | | |
| Example 79 Method 8 | 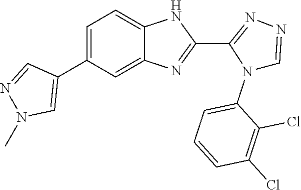 [SMILES: Cn1cc(-c2ccc3[nH]c(-c4nncn4-c4cccc(Cl)c4Cl)nc3c2)cn1] | white solid, yield: 11.5% | ¹H NMR (400 MHz, DMSO-d₆) δ 13.56 (s, 1H), 9.03 (s, 1H), 8.14 (s, 1H), 7.93 (d, J = 8.2 Hz, 1H), 7.86 (s, 1H), 7.81-7.78 (m, 1H), 7.61 (m, 2H), 7.44 (s, 2H), 3.85 (s, 3H). | 410 |
| | 2-(4-(2,3- | | | |
| | dichlorophenyl)-4H- | | | |
| | 1,2,4-triazol-3-yl)-5-(1- | | | |
| | methyl-1H-pyrazol-4- | | | |
| | yl)-1H- | | | |
| | benzo[d]imidazole | | | |
| Example 81 Method 8 | 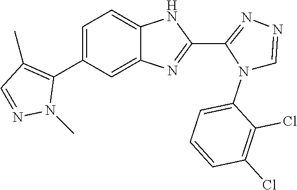 [SMILES: Cc1cnn(C)c1-c1ccc2[nH]c(-c3nncn3-c3cccc(Cl)c3Cl)nc2c1] | white solid, yield: 17.6% | ¹H NMR (400 MHz, DMSO-d₆) δ 9.08 (s, 1H), 7.93 (dd, J = 8.2, 1.3 Hz, 1H), 7.81 (dd, J = 8.0, 1.3 Hz, 1H), 7.65- 7.58 (m, 2H), 7.47 (s, 1H), 7.34 (s, 1H), 7.22 (d, J = 8.2 Hz, 1H), 3.67 (s, 3H), 1.94 (s, 3H). | 424 |
| | 2-(4-(2,3- | | | |
| | dichlorophenyl)-4H- | | | |
| | 1,2,4-triazol-3-yl)-5- | | | |
| | (1,4-dimethyl-1H- | | | |
| | pyrazol-5-yl)-1H- | | | |
| | benzo[d]imidazole | | | |

### Preparation of 5-(2-(4-(2,3-dichlorophenyl)-4H-1,2,4-triazol-3-yl)-1H-benzo[d]imidazol-5-yl)-3-methylisothiazole (80) Method 9

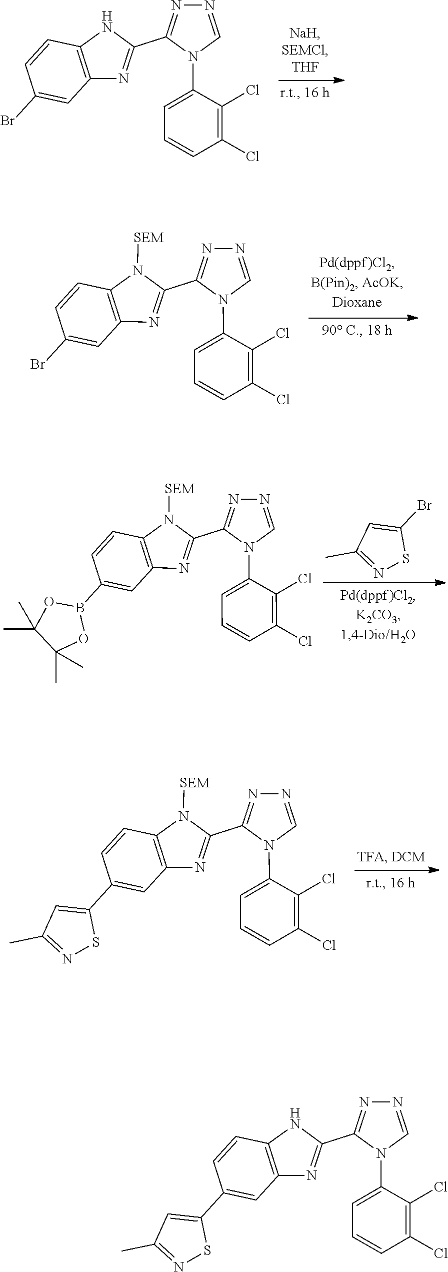

### Step 1. Preparation of 5-bromo-2-(4-(2,3-dichlorophenyl)-4H-1,2,4-triazol-3-yl)-1-((2-(trimethylsilyl)eth oxy)methyl)-1H-benzo[d]imidazole

To a solution of 5-bromo-2-(4-(2,3-dichlorophenyl)-4H-1,2,4-triazol-3-yl)-1H-benzo[d]imidazole (1.85 g, 0.0045 mol) and NaH (60% dispersion in mineral oil, 0.22 g, 0.0055 mol) in THF (15 mL). The reaction mixture was stirred at 0° C. for 30 mins, was later added SEM-Cl (0.76 g, 0.0046 mol). The reaction mixture was stirred at 25° C. for 18 h. The reaction mixture was extracted with EtOAc (100 mL×3), concentrated and the residue was purified through silica gel chromatography (PE/EA=5:1) to give 5-bromo-2-(4-(2,3-dichlorophenyl)-4H-1,2,4-triazol-3-yl)-1-((2-(trimethylsilyl)eth oxy)methyl)-1H-benzo[d]imidazole (1.2 g, 50% yield) as an oil.

MS (ESI) m/z 538 [M+H]⁺.

### Step 2. Preparation of 2-(4-(2,3-dichlorophenyl)-4H-1,2,4-triazol-3-yl)-5-(4,4,5,5-tetramethyl-1,3,2-dioxaborolan-2-yl)-1-((2-(trimethylsilyl)ethoxy)methyl)-1H-benzo[d]imidazole

The solution of 5-bromo-2-(4-(2,3-dichlorophenyl)-4H-1,2,4-triazol-3-yl)-1-((2-(trimethylsilyl) eth oxy) methyl)-1H-benzo[d]imidazole (1.163 g, 0. 0022 mol), B(Pin)₂ (1.100 g, 0.0043 mol), Pd(dppf)Cl₂ (0.158 g, 0.0002 mol) and AcOH (1.061 g, 0.0108 mol) in 1,4-dioxane (20 mL) was stirred at 90° C. for 18 h under N₂ atmosphere. Once the reaction was completed, the reaction was diluted with H₂O (50 mL), then extracted with EtOAc (50 mL×3). All the organic layers were combined and concentrated in vacuo. The residue was purified through silica gel chromatography (PE/EA=5:1) to give 2-(4-(2,3-dichlorophenyl)-4H-1,2,4-triazol-3-yl)-5-(4,4,5,5-tetramethyl-1,3,2-dioxaborolan-2-yl)-1-((2-(trimethylsilyl)ethoxy)methyl)-1H-benzo[d]imidazole (0.7 g, 55.10% yield) as an oil.

MS (ESI) m/z 586.2 [M+H]⁺.

### Step 3. Preparation of 5-(2-(4-(2,3-dichlorophenyl)-4H-1,2,4-triazol-3-yl)-1-((2-(trimethylsilyl)ethoxy)methyl)-1H-benzo[d]imidazol-5-yl)-3-methylisothiazole (3)

A mixture of 2-(4-(2,3-dichlorophenyl)-4H-1,2,4-triazol-3-yl)-5-(4,4,5,5-tetramethyl-1,3,2-dioxaborolan-2-yl)-1-((2-(trimethylsilyl)ethoxy)methyl)-1H-benzo[d]imidazole (100 mg, 0.1702 mmol), 5-bromo-3-methylisothiazole (36.36 mg, 0.2042 mmol), Pd(dppf)Cl₂ (18.68 mg, 0.0255 mmol), K₂CO₃ (70.57 mg, 0.5102 mmol) in 1,4-Dioxane/H₂O (10/2 mL) was stirred at 90° C. for 16 h under N₂ atmosphere. After cooling, the reaction mixture was diluted with water (60 mL), extracted with EtOAc (20 mL×3). All the organic layers were combined, dried over anhydrous Na₂SO₄ and concentrated under reduced pressure to give crude product that was purified through column chromatography on silica gel (EA/PE=1:1) to give the 5-(2-(4-(2,3-dichlorophenyl)-4H-1,2,4-triazol-3-yl)-1-((2-(trimethylsilyl)ethoxy)methyl)-1H-benzo[d]imidazol-5-yl)-3-methylisothiazole (60 mg, 82.49% yield) as an oil. MS (ESI) m/z 557.1 [M+H]⁺.

### Step 4. Preparation of 5-(2-(4-(2,3-dichlorophenyl)-4H-1,2,4-triazol-3-yl)-1H-benzo[d]imidazol-5-yl)-3-methylisothiazole

A mixture of 5-(2-(4-(2,3-dichlorophenyl)-4H-1,2,4-triazol-3-yl)-1-((2-(trimethylsilyl)ethoxy)methyl)-1H-benzo[d]imidazol-5-yl)-3-methylisothiazole (60 mg, 0.1074 mmol) in TFA/DCM (1/5 mL) was stirred for at 25° C. for 16 h. After reaction was completed, the reaction mixture was diluted with water (60 mL), extracted with DCM (20 mL×3). All the organic layers were combined, dried over anhydrous Na₂SO₄ and concentrated under reduced pressure to give crude product that was purified by perp-HPLC (column-Gemini-C18 150×21.2 mm, 5 um; Mobile phase: ACN-H₂O (0.10% TFA)) to afford the desired product (12.5 mg, 27.28% yield) as a white solid.

MS (ESI) m/z 427.2 [M+H]⁺.

¹H NMR (400 MHz, DMSO-d₆) δ 13.84 (s, 1H), 9.08 (s, 1H), 7.94 (d, J=7.8 Hz, 1H), 7.81 (d, J=8.2 Hz, 2H), 7.61 (t, J=8.2 Hz, 4H), 2.43 (s, 3H).

Examples (Compounds) 82, 83 were synthesized using a method similar to that used in Example 80.

| Example | | | | |
|---|---|---|---|---|
| No. | | | | MS |
| (Compound | | Appearance | | (m/z) |
| No.) | | and | | [M + |
| Method | Structure and Name | Yield | ¹H NMR Data | H]⁺ |
| Example 82 Method 9 | 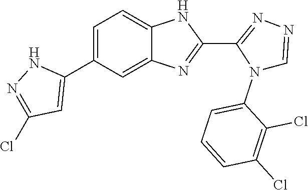 [SMILES: Clc1cc(-c2ccc3[nH]c(-c4nncn4-c4cccc(Cl)c4Cl)nc3c2)[nH]n1] | white solid, yield: 19.3% | ¹H NMR (400 MHz, DMSO-d₆) δ 9.02 (s, 1H), 7.90 (d, J = 8.0 Hz, 1H), 7.85 (s, 1H), 7.76 (dd, J = 8.0, 1.2 Hz, 1H), 7.57 (t, J = 8.0 Hz, 2H), 7.52 (d, J = 3.4 Hz, 1H), 6.76 (s, 1H). | 430 |
| | 5-(3-chloro-1H-pyrazol- | | | |
| | 5-yl)-2-(4-(2,3- | | | |
| | dichlorophenyl)-4H- | | | |
| | 1,2,4-triazol-3-yl)-1H- | | | |
| | benzo[d]imidazole | | | |
| Example 100 Method 9 | 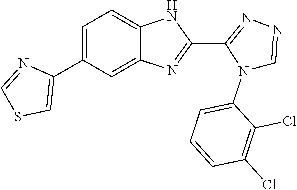 [SMILES: Clc1cccc(-n2cnnc2-c2nc3cc(-c4cscn4)ccc3[nH]2)c1Cl] | white solid, yield: 22.3% | ¹H NMR (400 MHz, DMSO-d₆) δ 13.68 (s, 1H), 9.19 (d, J = 1.8 Hz, 1H), 9.07 (s, 1H), 8.18 (d, J = 1.8 Hz, 1H), 7.94 (dd, J = 8.2, 1.4 Hz, 1H), 7.89 (s, 1H), 7.81 (m, 1H), 7.62 (m, 1H), 7.54 (d, J = 8.6 Hz, 1H). | 413 |
| | 4-(2-(4-(2,3- | | | |
| | dichlorophenyl)-4H- | | | |
| | 1,2,4-triazol-3-yl)-1H- | | | |
| | benzo[d]imidazol-5- | | | |
| | yl)thiazole | | | |

### Preparation of 5-(2,3-dichlorophenyl)-4-[5-(trifluoromethyl)-1H-benzimidazol-2-yl]-1H-pyridin-2-one (40) Method 10

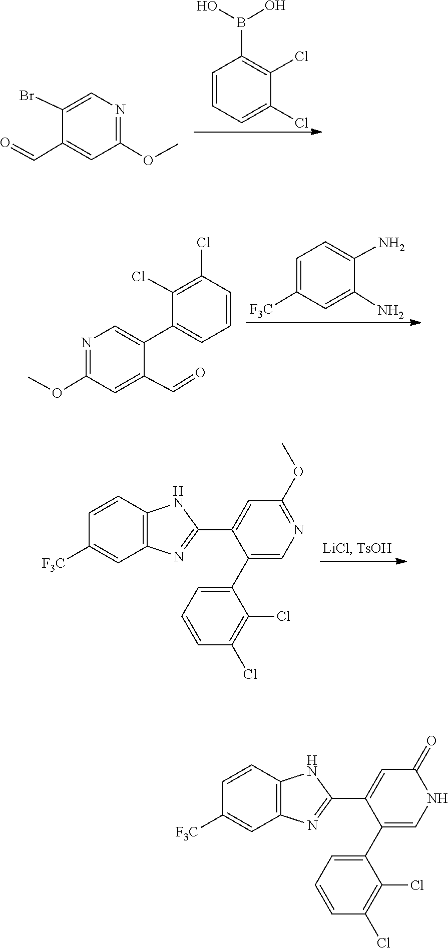

### Step 1. 5-(2,3-dichlorophenyl)-2-methoxyisonicotinaldehyde

To a solution of 5-bromo-2-methoxy-pyridine-4-carbaldehyde (1.00 eq, 400 mg, 1.85 mmol) in DMF (9 mL) was added (2,3-dichlorophenyl)boronic acid (1.00 eq, 353 mg, 1.85 mmol), Na₂CO₃ (3.00 eq, 589 mg, 5.55 mmol) and Pd(dppf)Cl₂ (0.100 eq, 135 mg, 0.185 mmol). The mixture was stirred at 110° C. for 12 h under N₂. The mixture was diluted with EtOAc and washed with brine. The organic phase was separated, dried over anhydrous Na₂SO₄ and concentrated under vacuum. The residue was purified by silica gel flash column chromatography (PE/EA=20/1) to afford 5-(2,3-dichlorophenyl)-2-methoxy-pyridine-4-carbaldehyde (351 mg, 1.24 mmol, 67.19% yield). MS (ESI) m/z 282 [M+H]⁺.

### Step 2. 2-(5-(2,3-dichlorophenyl)-2-methoxypyridin-4-yl)-5-(trifluoromethyl)-1H-benzo[d]imidazole

To a solution of 5-(2,3-dichlorophenyl)-2-methoxy-pyridine-4-carbaldehyde (1.00 eq, 350 mg, 1.24 mmol) in water (12 mL) and THF (2 mL) was added 4-(trifluoromethyl)benzene-1,2-diamine (1.00 eq, 219 mg, 1.24 mmol). The reaction mixture was stirred for 20 min before K₂CO₃ (2.00 eq, 343 mg, 2.48 mmol) was added. The reaction mixture was stirred for another 10 min, then evaporated the THF. 12 (1.00 eq, 315 mg, 1.24 mmol) and KI (0.250 eq, 51 mg, 0.310 mmol) were added. The mixture was heated to 80° C. and stirred for 2 h. The reaction mixture was quenched with saturated Na₂S₂O₃ and extracted with EtOAc. The combined organic phase was dried over anhydrous Na₂SO₄ and concentrated to give the crude product which was used in next step without further purification. MS (ESI) m/z 438 [M+H]⁺.

### Step 3. 5-(2,3-dichlorophenyl)-4-(5-(trifluoromethyl)-1H-benzo[d]imidazol-2-yl)pyridin-2 (1H)-one

To a solution of 2-[5-(2,3-dichlorophenyl)-2-methoxy-4-pyridyl]-5-(trifluoromethyl)-1H-benzimidazole (1.00 eq, 543 mg, 1.24 mmol) in DMF (3 mL) was added TsOH (5.00 eq, 1068 mg, 6.20 mmol) and LiCl (5.00 eq, 263 mg, 6.20 mmol). The mixture was stirred at 120° C. for 30 min. The reaction mixture was diluted with EtOAc and washed with brine. The organic phase was separated, dried over anhydrous Na₂SO₄ and concentrated under vacuum. The residue was purified by prep-HPLC to give 5-(2,3-dichlorophenyl)-4-[5-(trifluoromethyl)-1H-benzimidazol-2-yl]-1H-pyridin-2-one (130 mg, 0.306 mmol, 24.7% yield) as a white solid.

MS (ESI) m/z 424 [M+H]⁺.

¹H NMR (400 MHz, DMSO) δ 13.26 (s, 1H), 12.19 (s, 1H), 7.75 (s, 1H), 7.64 (d, J=8.5 Hz, 1H), 7.58 (dd, J=6.9, 2.6 Hz, 1H), 7.53 (s, 1H), 7.48 (d, J=8.5 Hz, 1H), 7.41-7.33 (m, 2H), 6.94 (s, 1H).

Examples (Compounds) 21, 22, 23, 34, 35, 37, 38, 39, 42, 43, 44, 48, 54, 57, 60, 61, 64, 65, 75, 76, 86, 92, 98 were synthesized using a method similar to that used in Example 40.

| Example | | | | |
|---|---|---|---|---|
| No. | | | | |
| (Compound | | Appearance | | |
| No.) | | and | | MS (m/z) |
| Method | Structure and Name | Yield | ¹H NMR Data | [M + H]⁺ |
| Example 21 Method 10 | 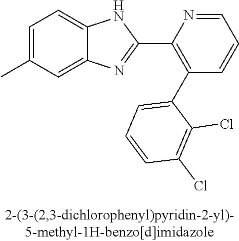 [SMILES: Cc1ccc2[nH]c(-c3ncccc3-c3cccc(Cl)c3Cl)nc2c1] | brown solid, yield: 6.8% | ¹H NMR (400 MHz, Methanol-d₄) δ 8.77 (dd, J = 4.8, 1.6 Hz, 1H), 7.82 (dd, J = 7.6, 1.6 Hz, 1H), 7.65-7.47 (m, 2H), 7.42-7.25 (m, 4H), 7.04 (dd, J = 8.0, 1.6 Hz, 1H), 2.42 (s, 3H). | 354 |
| Example 22 Method 10 | 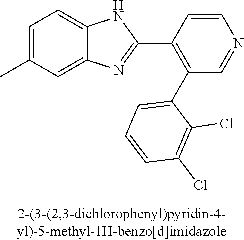 [SMILES: Cc1ccc2[nH]c(-c3ccncc3-c3cccc(Cl)c3Cl)nc2c1] | brown solid, yield: 21.4% | ¹H NMR (400 MHz, Methanol-d4) δ 8.77 (d, J = 5.2 Hz, 1H), 8.61 (s, 1H), 7.88 (d, J = 5.2 Hz, 1H), 7.57 (dd, J = 5.2, 4.4 Hz, 1H), 7.45-7.24 (m, 4H), 7.08 (dd, J = 8.4, 1.6 Hz, 1H), 2.43 (s, 3H). | 354 |
| Example 23 Method 10 | 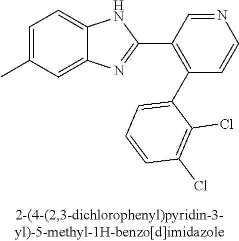 [SMILES: Cc1ccc2[nH]c(-c3cnccc3-c3cccc(Cl)c3Cl)nc2c1] | brown solid, yield: 12.1% | ¹H NMR (400 MHz, Methanol-d₄) δ 8.99 (s, 1H), 8.76 (d, J = 5.2 Hz, 1H), 7.62- 7.50 (m, 2H), 7.42- 7.23 (m, 4H), 7.07 (dd, J = 8.0, 1.6 Hz, 1H), 2.43 (s, 3H). | 354 |
| Example 34 Method 10 | 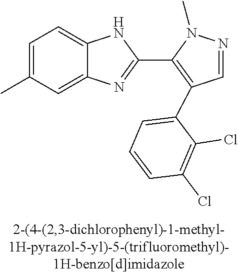 [SMILES: Cc1ccc2[nH]c(-c3c(-c4cccc(Cl)c4Cl)cnn3C)nc2c1] | off-white solid, yield: 22.9% | ¹H NMR (400 MHz, CD₃OD) δ 7.92 (s, 1H), 7.80 (s, 1H), 7.73 (d, J = 8.4 Hz, 1H), 7.58 (d, J = 8.4 Hz, 1H), 7.49 (d, J = 7.2 Hz, 1H), 7.24 (t, J = 7.6 Hz, 1H), 7.18 (d, J = 7.6 Hz, 1H), 4.14 (s, 3H). | 357 |
| Example 35 Method 10 | 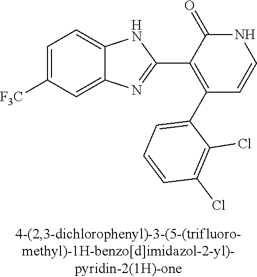 [SMILES: O=c1[nH]ccc(-c2cccc(Cl)c2Cl)c1-c1nc2cc(C(F)(F)F)ccc2[nH]1] | off-white solid, yield: 37.9% | ¹H NMR (400 MHz, DMSO-d₆) δ 13.03 (s, 1H), 12.62 (s, 1H), 7.99-7.60 (m, 3H), 7.58-7.30 (m, 3H), 7.22 (dd, J = 7.6, 1.6 Hz, 1H), 6.31 (d, J = 6.4 Hz, 1H). | 424 |
| Example 37 Method 10 | 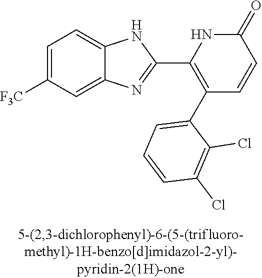 [SMILES: O=c1ccc(-c2cccc(Cl)c2Cl)c(-c2nc3cc(C(F)(F)F)ccc3[nH]2)[nH]1] | white solid, yield: 15% | ¹H NMR (400 MHz, MeOD) δ 7.73 (s, 1H), 7.58 (s, 1H), 7.55 (s, 1H), 7.48- 7.39 (m, 2H), 7.22- 7.09 (m, 2H), 6.74 (d, J = 9.1 Hz, 1H). | 424 |
| Example 38 Method 10 | 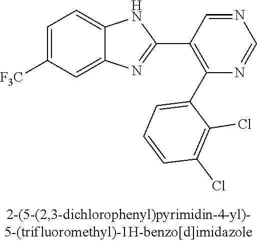 [SMILES: FC(F)(F)c1ccc2[nH]c(-c3cncnc3-c3cccc(Cl)c3Cl)nc2c1] | white solid, yield: 10.3% | ¹H NMR (400 MHz, Methanol-d₄) δ 9.31 (s, 1H), 9.23 (s, 1H), 7.75 (s, 1H), 7.63- 7.52 (m, 2H), 7.48- 7.31 (m, 3H). | 409 |
| Example 39 Method 10 | 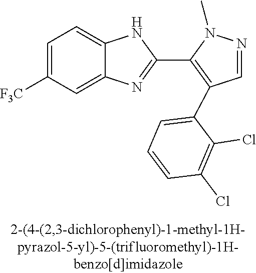 [SMILES: Cn1ncc(-c2cccc(Cl)c2Cl)c1-c1nc2cc(C(F)(F)F)ccc2[nH]1] | off-white solid, yield: 22.9% | ¹H NMR (400 MHz, CD₃OD) δ 7.92 (s, 1H), 7.80 (s, 1H), 7.73 (d, J = 8.4 Hz, 1H), 7.58 (d, J = 8.4 Hz, 1H), 7.49 (d, J = 7.2 Hz, 1H), 7.24 (t, J = 7.6 Hz, 1H), 7.18 (d, J = 7.6 Hz, 1H), 4.14 (s, 3H) | 411 |
| Example 42 Method 10 | 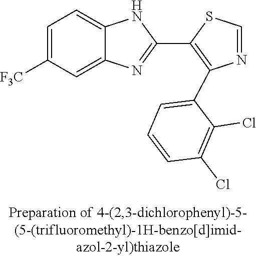 [SMILES: FC(F)(F)c1ccc2[nH]c(-c3scnc3-c3cccc(Cl)c3Cl)nc2c1] | gray solid, yield: 1.3% | ¹H NMR (400 MHz, CDCl₃) δ 8.99 (s, 1H), 7.84 (s, 1H), 7.64 (m, 2H), 7.52 (d, J = 8.2 Hz, 1H), 7.45 (d, J = 7.4 Hz, 1H), 7.38 (t, J = 7.6 Hz, 1H). | 414 |
| Example 43 Method 10 | 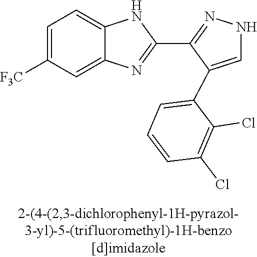 [SMILES: FC(F)(F)c1ccc2[nH]c(-c3n[nH]cc3-c3cccc(Cl)c3Cl)nc2c1] | white solid, yield: 50.3% | ¹H NMR (400 MHz, CDCl₃) δ 7.79 (d, J = 32.4 Hz, 2H), 7.56 (t, J = 16.0 Hz, 2H), 7.15 (s, 2H), 6.98 (s, 1H). | 397 |
| Example 44 Method 10 | 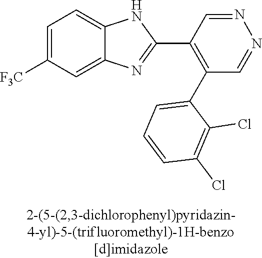 [SMILES: FC(F)(F)c1ccc2[nH]c(-c3cnncc3-c3cccc(Cl)c3Cl)nc2c1] | white solid, yield: 17.9% | ¹H NMR (400 MHz, Methanol-d₄) δ 9.77 (s, 1H), 9.37 (s, 1H), 7.93-7.68 (m, 3H), 7.59 (s, 1H), 7.50- 7.39 (m, 2H). | 409 |
| Example 48 Method 10 | 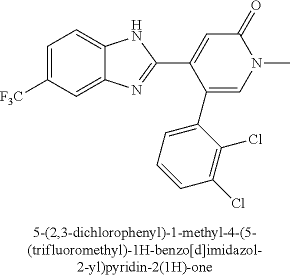 [SMILES: Cn1cc(-c2cccc(Cl)c2Cl)c(-c2nc3cc(C(F)(F)F)ccc3[nH]2)cc1=O] | white solid, yield: 24.7% | ¹H NMR (400 MHz, DMSO-d₆) δ 7.93 (s, 1H), 7.75 (s, 1H), 7.65 (d, J = 8.1 Hz, 1H), 7.61-7.58 (m, 1H), 7.49 (d, J = 8.4 Hz, 1H), 7.43-7.37 (m, 2H), 7.00 (s, 1H), 3.55 (s, 3H). | 438 |
| Example 54 Method 10 | 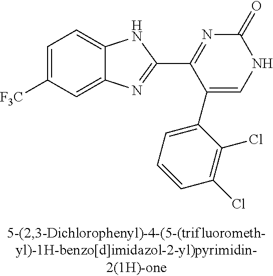 [SMILES: O=c1nc(-c2nc3cc(C(F)(F)F)ccc3[nH]2)c(-c2cccc(Cl)c2Cl)c[nH]1] | white solid, yield: 13% | ¹H NMR (400 MHz, DMSO-d6) δ 8.20 (s, 1H), 7.86-7.61 (m, 3H), 7.61-7.34 (m, 3H). | 425 |
| Example 57 Method 10 | 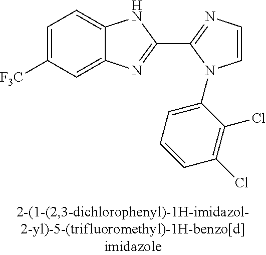 [SMILES: FC(F)(F)c1ccc2[nH]c(-c3nccn3-c3cccc(Cl)c3Cl)nc2c1] | white solid, yield: 11% | ¹H NMR (400 MHz, DMSO-d₆) δ 13.51 (bs, 1H), 7.87 (dd, J = 8.4 Hz, J = 1.2 Hz, 1H), 7.71 (s, 1H), 7.68-7.64 (m, 2H), 7.63-7.59 (m, 1H), 7.56 (t, J = 8.0 Hz, 1H), 7.52-7.44 (m, 1H), 7.43 (d, J = 1.2 Hz, 1H). | 397 |
| Example 60 Method 10 | 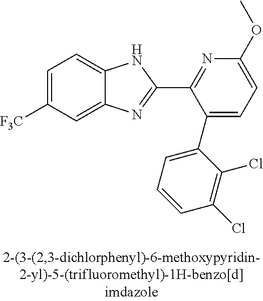 [SMILES: COc1ccc(-c2cccc(Cl)c2Cl)c(-c2nc3cc(C(F)(F)F)ccc3[nH]2)n1] | White solid, yield: 36.8% | ¹H NMR (400 MHz, DMSO-d₆) δ 13.01 (d, J = 10.8 Hz, 1H), 7.76 (dt, J = 8.5, 2.0 Hz, 2H), 7.69-7.61 (m, 2H), 7.57-7.50 (m, 1H), 7.41 (t, J = 7.8 Hz, 1H), 7.35- 7.32 (m, 1H), 7.10 (dd, J = 8.4, 4.1 Hz, 1H), 4.14 (s, 3H). | 438 |
| Example 61 Method 10 | 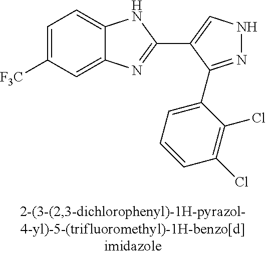 [SMILES: FC(F)(F)c1ccc2[nH]c(-c3c[nH]nc3-c3cccc(Cl)c3Cl)nc2c1] | off-white solid, yield: 17.0% | ¹H NMR (400 MHz, DMSO-d₆) δ 13.57 (s, 1H), 12.73 (s, 1H), 8.37 (d, J = 99.6 Hz, 1H), 7.76 (t, J = 11.6 Hz, 2H), 7.62 (d, J = 8.0 Hz, 1H), 7.57-7.36 (m, 3H). | 397 |
| Example 64 Method 10 | 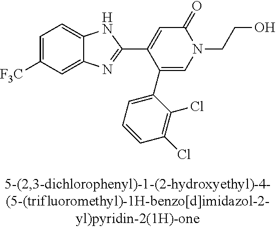 [SMILES: O=c1cc(-c2nc3cc(C(F)(F)F)ccc3[nH]2)c(-c2cccc(Cl)c2Cl)cn1CCO] | white solid, yield: 41.0% | ¹H NMR (400 MHz, DMSO-d₆) δ 13.27 (s, 1H), 7.75 (q, J = 16.4 Hz, 2H), 7.63- 7.51 (m, 2H), 7.45- 7.34 (m, 2H), 6.99 (s, 1H), 4.94 (t, J = 5.4 Hz, 1H), 4.17- 3.98 (m, 2H), 3.71 (q, J = 5.6 Hz, 2H). | 468 |
| Example 65 Method 10 | 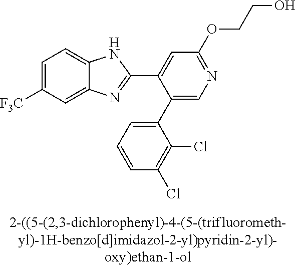 [SMILES: OCCOc1cc(-c2nc3cc(C(F)(F)F)ccc3[nH]2)c(-c2cccc(Cl)c2Cl)cn1] | white solid, yield: 8.3% | ¹H NMR (400 MHz, DMSO-d₆) δ 13.27 (s, 1H), 8.21 (s, 1H), 7.82-7.59 (m, 3H), 7.45-7.32 (m, 3H), 4.88 (t, J = 5.4 Hz, 1H), 4.43 (td, J = 4.9, 2.2 Hz, 2H), 3.79 (q, J = 5.3 Hz, 2H). | 468 |
| Example 75 Method 10 | 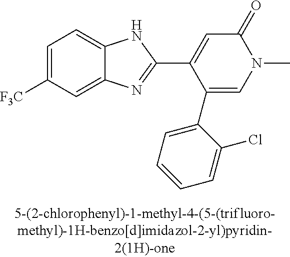 [SMILES: Cn1cc(-c2ccccc2Cl)c(-c2nc3cc(C(F)(F)F)ccc3[nH]2)cc1=O] | white solid, yield: 25% | ¹H NMR (400 MHz, DMSO) δ 13.18 (s, 1H), 7.88 (s, 1H), 7.74 (s, 1H), 7.63 (d, J = 8.5 Hz, 1H), 7.46 (dd, J = 7.5, 1.8 Hz, 2H), 7.42, 7.35 (m, 1H), 7.31 (ddd, J = 12.7, 7.5, 1.8 Hz, 2H), 6.94 (s, 1H), 3.56 (s, 3H). | 404 |
| Example 76 Method 10 | 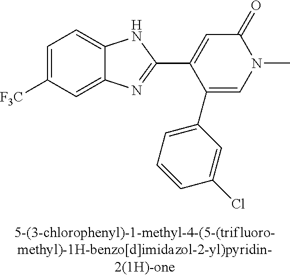 [SMILES: Cn1cc(-c2cccc(Cl)c2)c(-c2nc3cc(C(F)(F)F)ccc3[nH]2)cc1=O] | white solid, yield: 14.8% | ¹H NMR (400 MHz, DMSO) δ 13.02 (s, 1H), 7.98 (s, 1H), 7.81 (d, J = 95.6 Hz, 2H), 7.59-7.44 (m, 1H), 7.30 (d, J = 8.1 Hz, 2H), 7.10 (d, J = 8.2 Hz, 2H), 6.81 (s, 1H), 3.57 (s, 3H). | 404 |
| Example 86 Method 10 | 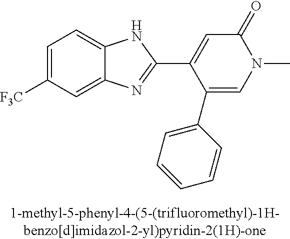 [SMILES: Cn1cc(-c2ccccc2)c(-c2nc3cc(C(F)(F)F)ccc3[nH]2)cc1=O] | white solid, yield: 16% | ¹H NMR (400 MHz, DMSO) δ 12.94 (s, 1H), 7.97 (s, 1H), 7.95-7.58 (m, 2H), 7.50 (d, J = 8.5 Hz, 1H), 7.28-7.18 (m, 3H), 7.11-7.05 (m, 2H), 6.78 (s, 1H), 3.58 (s, 3H). | 370 |
| Example 92 Method 10 | 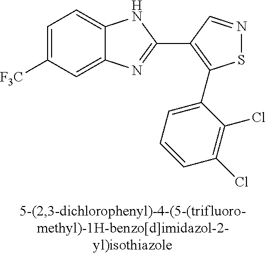 [SMILES: FC(F)(F)c1ccc2[nH]c(-c3cnsc3-c3cccc(Cl)c3Cl)nc2c1] | yellow solid, yield: 43.5% | ¹H NMR (400 MHz, DMSO-d₆) δ 13.19 (s, 1H), 9.22 (s, 1H), 7.88-7.80 (m, 2H), 7.72 (d, J = 8.4 Hz, 1H), 7.57 (d, J = 1.6 Hz, 1H), 7.51 (t, J = 7.8 Hz, 2H). | 414 |
| Example 98 Method 10 | 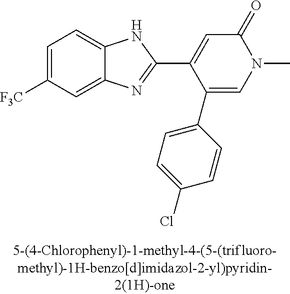 [SMILES: Cn1cc(-c2ccc(Cl)cc2)c(-c2nc3cc(C(F)(F)F)ccc3[nH]2)cc1=O] | white solid, yield: 10% | ¹H NMR (400 MHz, DMSO) δ 12.94 (s, 1H), 7.97 (s, 1H), 7.95-7.58 (m, 2H), 7.50 (d, J = 8.5 Hz, 1H), 7.28-7.18 (m, 3H), 7.11-7.05 (m, 2H), 6.78 (s, 1H), 3.58 (s, 3H). | 404 |

### Preparation of 6-[(1-oxo-2-isoquinolyl)methyl]-3H-1,3-benzoxazol-2-one (45)

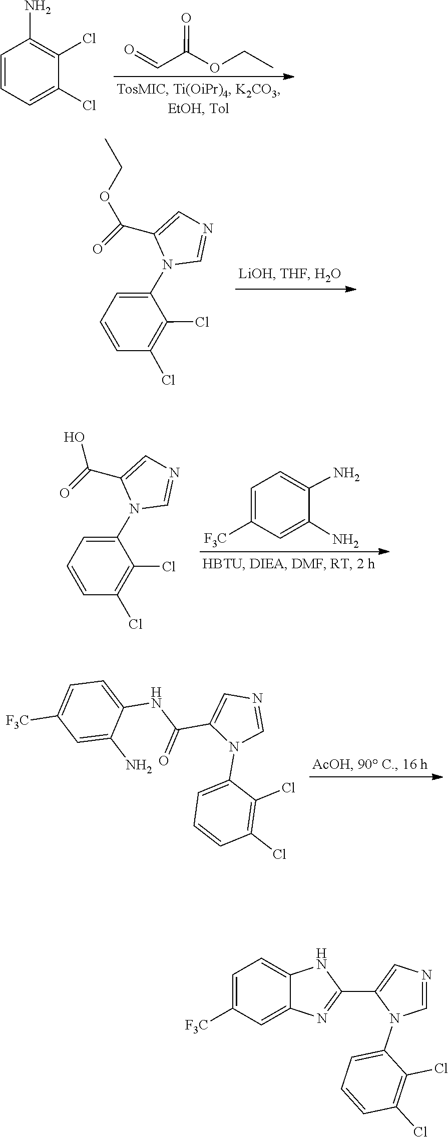

### Step 1. ethyl 1-(2,3-dichlorophenyl)-1H-imidazole-5-carboxylate

To a solution of ethyl 2-oxoacetate (0.83 mL, 4.18 mmol) in toluene (20 mL) was added 2,3-dichloroaniline (678 mg, 4.18 mmol) and Ti(OiPr)₄ (2.5 mL, 8.37 mmol). The reaction mixture was heated to 70° C. and stirred for 4 h. The resulting mixture was evaporated to remove the solvent, the residue was then dissolved in EtOH. To the solution was added K₂CO₃ (1.73 g, 12.6 mmol) and TosMIC (980 mg, 5.02 mmol). The mixture was heated to 80° C. and stirred for 5 h. The mixture was diluted with EtOAc and filtered. The filtrate was dried over anhydrous sodium sulfate and concentrated under vacuum. The residue was purified by silica gel flash column chromatography (PE/EtOAc=1/5) to afford ethyl 1-(2,3-dichlorophenyl)-1H-imidazole-5-carboxylate (1.1 g, 92.6% yield) as a yellow solid. MS (ESI) m/z 285 [M+H]⁺.

### Step 2. 1-(2,3-dichlorophenyl)-1H-imidazole-5-carboxylic acid

To a solution of ethyl 1-(2,3-dichlorophenyl)-1H-imidazole-5-carboxylate (1.1 g, 3.8 mmol) in THF/H₂O (10:1, 22 mL) was added lithium hydroxide monohydrate (1.6 g, 38.5 mmol) at r.t. The mixture was stirred at r.t. for 16 h. After the reaction was completed, the reaction mixture was diluted with water and adjusted the pH to 7. The mixture was diluted with EtOAc and washed with water. The organic layer was separated, dried over anhydrous sodium sulfate and concentrated under vacuum to give 1-(2,3-dichlorophenyl)-1H-imidazole-5-carboxylic acid (0.9 g, 90.4% yield) as a yellow solid, which was used in the next step without further purification. MS (ESI) m/z 257 [M+H]⁺.

### Step 3. N-(2-amino-4-(trifluoromethyl)phenyl)-1-(2,3-dichlorophenyl)-1H-imidazole-5-carboxamide

To a solution of 1-(2,3-dichlorophenyl)-1H-imidazole-5-carboxylic acid (0.9 g, 3.65 mmol) in DMF (20 mL) were added HBTU (2.8 g, 7.3 mmol), 4-(trifluoromethyl)benzene-1,2-diamine (0.78 g, 4.4 mmol) and DIEA (1.43 g, 11.1 mmol). The mixture was stirred at 25° C. for 2 h. After the reaction was completed, the mixture was diluted with EtOAc and washed with water. The organic layer was separated, dried over anhydrous sodium sulfate and concentrated under vacuum to afford N-(2-amino-4-(trifluoromethyl)phenyl)-1-(2,3-dichlorophenyl)-1H-imidazole-5-carboxamide (1.5 g) as a red oil, which was used in the next step without further purification. MS (ESI) m/z 415 [M+H]f.

### Step 4. 2-(1-(2,3-dichlorophenyl)-1H-imidazol-5-yl)-5-(trifluoromethyl)-1H-benzo[d]imidazole

To a solution of N-(2-amino-4-(trifluoromethyl)phenyl)-1-(2,3-dichlorophenyl)-1H-imidazole-5-carboxamide (400 mg, crude) in AcOH (20 mL) was heated to 90° C. and stirred for 16 h. After the reaction was completed, the mixture was diluted with water and adjusted the pH to 7. The mixture was diluted with EtOAc and washed with water. The organic layer was separated, dried over anhydrous sodium sulfate and concentrated in vacuo. The residue was purified by prep-HPLC to give 2-(1-(2,3-dichlorophenyl)-1H-imidazol-5-yl)-5-(trifluoromethyl)-1H-benzo[d]imidazole (5.0 mg, 1% yield) as a yellow powder.

MS (ESI) m/z 397 [M+H]⁺.

¹H NMR (400 MHz, DMSO-d₆) (13.27 (bs, 1H), 7.86 (dd, J=8.0 Hz, J=1.2 Hz 1H), 7.72 (s, 1H), 7.69-7.59 (m, 3H), 7.56 (t, J=8.0 Hz, 1H), 7.52-7.41 (m, 2H).

### Preparation of 2-(4-(2,3-dichlorophenyl)pyridin-3-yl)-5-(trifluoromethyl)-1H-indole-3-carbonitrile (47)

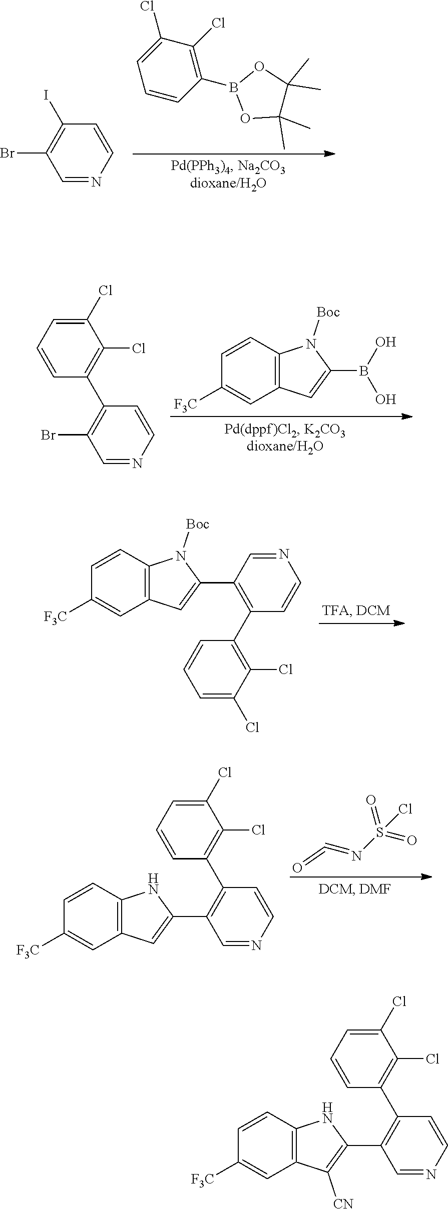

### Step 1. 3-bromo-4-(2,3-dichlorophenyl)pyridine

To a solution of 3-bromo-4-iodopyridine (4 g, 14.1 mmol) in dioxane/H₂O (100 mL/20 mL) was added 2-(2,3-dichlorophenyl)-4,4,5,5-tetramethyl-1,3,2-dioxaborolane (4.6 g, 16.9 mmol), Pd(PPh₃)₄ (1.63 g, 1.41 mmol) and Na₂CO₃ (2.99 g, 28.2 mmol). The reaction mixture was stirred at 100° C. for 16 h under N₂. The mixture was diluted with water (50 mL), extracted with EtOAc (50 mL×3). The combined organic layers were dried over Na₂SO₄ and filtered. The filtrate was concentrated and purified by FCC (PE/EA=5/1) to give 3-bromo-4-(2,3-dichlorophenyl)pyridine (2.1 g, 50%) as a yellow oil. MS (ESI) m/z 302 [M+H]⁺.

### Step 2. tert-butyl 2-(4-(2,3-dichlorophenyl)pyridin-3-yl)-5-(trifluoromethyl)-1H-indole-1-carboxylate

To a solution of 3-bromo-4-(2,3-dichlorophenyl)pyridine (300 mg, 1 mmol) in dioxane/H₂O (15 mL/3 mL) was added (1-(tert-butoxycarbonyl)-5-(trifluoromethyl)-1H-indol-2-yl)boronic acid (492 mg, 1.49 mmol), K₂CO₃ (412 mg, 2.99 mmol) and Pd(dppf)Cl₂ (73 mg, 0.1 mmol). The mixture was stirred at 90° C. for 2 h under N₂. To the mixture was added H₂O (20 mL), then the reaction mixture was extracted with EtOAc (10 mL×2). The combined organic layers were dried over Na₂SO₄ and concentrated. The residue was purified by FCC (PE/EA=1/1) to give tert-butyl 2-(4-(2,3-dichlorophenyl)pyridin-3-yl)-5-(trifluoromethyl)-1H-indole-1-carboxylate (120 mg, 24%) as a yellow solid. MS (ESI) m/z 507 [M+H]⁺.

### Step 3. 2-(4-(2,3-dichlorophenyl)pyridin-3-yl)-5-(trifluoromethyl)-1H-indole

A solution of tert-butyl 2-(4-(2,3-dichlorophenyl)pyridin-3-yl)-5-(trifluoromethyl)-1H-indole-1-carboxylate (450 mg, 0.89 mmol) in DCM/TFA (10 mL/10 mL) was stirred at r.t. for 2 h. The reaction mixture was concentrated and adjusted pH=9 with sat. NaHCO₃, then extracted with DCM (10 mL×2). The combined organic layers were dried over Na₂SO₄ and concentrated. The residue was purified by FCC (PE/EA=1/1) to give 2-(4-(2,3-dichlorophenyl)pyridin-3-yl)-5-(trifluoromethyl)-1H-indole (250 mg, 70%) as a yellow solid.

MS (ESI) m/z 407 [M+H]⁺.

### Step 4. 2-(4-(2,3-dichlorophenyl)pyridin-3-yl)-5-(trifluoromethyl)-1H-indole-3-carbonitrile

To a solution of 2-(4-(2,3-dichlorophenyl)pyridin-3-yl)-5-(trifluoromethyl)-1H-indole (200 mg, 0.49 mmol) in DCM (15 mL) was added sulfurisocyanatidic chloride (347 mg, 2.46 mmol). The mixture was stirred at rt for 16 h. Then DMF (2 mL) was added to the mixture and stirred at r.t. for 1 hour. To the reaction mixture was added H₂O (10 mL), then extracted with DCM (10 mL×2). The combined organic layers were dried over Na₂SO₄ and concentrated. The residue was purified by prep-HPLC to give 2-(4-(2,3-dichlorophenyl)pyridin-3-yl)-5-(trifluoromethyl)-1H-indole-3-carbonitrile (56.4 mg, 27%) as an off-white solid.

MS (ESI) m/z 432 [M+H]⁺.

¹H NMR (400 MHz, DMSO-d₆) δ 12.93 (s, 1H), 8.96 (s, 1H), 8.90 (d, J=5.2 Hz, 1H), 7.90 (s, 1H), 7.74-7.56 (m, 4H), 7.43-7.27 (m, 2H).

### Preparation of 5-(2,3-Dichlorophenyl)-4-(5-(2-hydroxypropan-2-yl)-1H-benzo[d]imidazol-2-yl)-1-methylpyridin-2 (1H)-one (83)

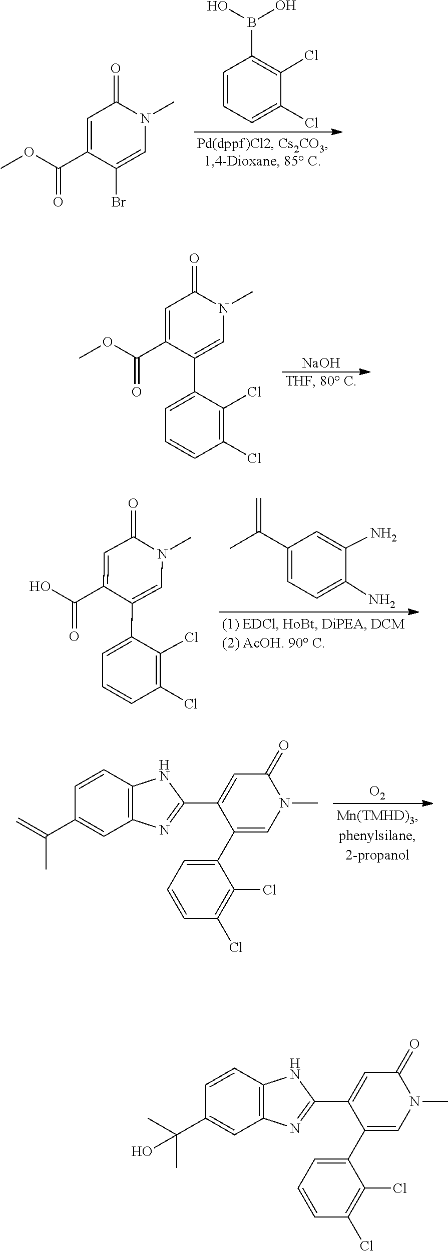

### Step 1. Methyl 5-(2,3-dichlorophenyl)-1-methyl-2-oxo-1,2-dihydropyridine-4-carboxylate

To a solution of methyl 5-bromo-1-methyl-2-oxo-pyridine-4-carboxylate (1.00 eq, 1040 mg, 4.23 mmol) in 1,4-dioxane (30 mL) was added (2,3-dichlorophenyl)boronic acid (1.00 eq, 807 mg, 4.23 mmol), cesium carbonate (3.00 eq, 4131 mg, 12.7 mmol) and Pd(dppf)Cl₂ (0.100 eq, 309 mg, 0.423 mmol) under Ar. Then the reaction mixture was stirred at 100° C. for 2 h. Once the reaction was completed, the reaction mixture was diluted with DCM and washed with water. The organic layer was dried over Na₂SO₄, filtered and concentrated under reduced pressure. The residue was purified by column chromatography (PE/EA=1/1) to yield the product (314 mg, 25%). MS (ESI) m/z 312 [M+H]⁺.

### Step 2. 5-(2,3-Dichlorophenyl)-1-methyl-2-oxo-pyridine-4-carboxylic acid

To a solution of methyl 5-(2,3-dichlorophenyl)-1-methyl-2-oxo-pyridine-4-carboxylate (1.00 eq, 314 mg, 1.01 mmol) in THF (4 mL) was added 15% NaOH (2.0 mL) and the mixture was stirred at 85° C. for 2 h. The reaction progress was monitored by LC/MS. Once completed, the mixture was diluted with water and washed with DCM. The water layer was acidified to pH=2 using 4 N HCl. The mixture was extracted with EtOAc, dried over Na₂SO₄, filtered and concentrated under reduced pressure. The residue was used directly in the next step. MS (ESI) m/z 298 [M+H]⁺.

### Step 3. 5-(2,3-Dichlorophenyl)-4-(5-isopropenyl-1H-benzimidazol-2-yl)-1-methyl-pyridin-2-one

To a solution of 4-isopropenylbenzene-1,2-diamine (1.00 eq, 149 mg, 0.503 mmol) and 5-(2,3-dichlorophenyl)-1-methyl-2-oxo-pyridine-4-carboxylic acid (1.00 eq, 150 mg, 0.503 mmol). in DCM (20 mL) were added HOBT (1.30 eq, 0.653 mmol), EDCI (1.30 eq, 0.653 mmol) and DIPEA (10.00 eq, 5.03 mmol). The mixture was stirred at 25° C. for 2 h. After the reaction was completed, the mixture was diluted with DCM and washed with water. The organic layer was separated, dried over anhydrous sodium sulfate and concentrated under vacuum to oil, which was used in the next step without further purification. To a solution of crude product prepared before in AcOH (20 mL) was heated to 90° C. and stirred for 16 h. After the reaction was completed, the mixture was diluted with water and adjusted the pH to 7. The mixture was diluted with EtOAc and washed with water. The organic layer was separated, dried over anhydrous sodium sulfate and concentrated in vacuo. The residue was purified by prep-HPLC to go give the product (70 mg, 34%). MS (ESI) m/z 410 [M+H]⁺.

### Step 4. 5-(2,3-Dichlorophenyl)-4-[5-(1-hydroxy-1-methyl-ethyl)-1H-benzimidazol-2-yl]-1-methyl-pyridin-2-one

To a solution of 5-(2,3-dichlorophenyl)-4-(5-isopropenyl-1H-benzimidazol-2-yl)-1-methyl-pyridin-2-one (1.00 eq, 38 mg, 0.0926 mmol) in 2-propanol (5 mL) was added phenylsilane (2.00 eq, 0.023 mL, 0.185 mmol) and Tris(2,2,6,6-tetramethyl-3,5-heptanedionato)manganese(III) (0.100 eq, 5.6 mg, 0.00926 mmol) at 0° C. under 02 atmosphere. Then the reaction mixture was stirred for 2 h. Once completed, the reaction mixture was quenched by water and extracted with DCM. The combined organic layers were dried over Na₂SO₄, filtered and concentrated under reduced pressure. The residue was purified by prep-HPLC to yield the product (1 mg, 2.5%) as a white solid.

MS (ESI) m/z 428 [M+H]⁺.

¹H NMR (400 MHz, CDCl₃) δ 8.01 (s, 1H), 7.54 (d, J=6.4 Hz, 2H), 7.31 (m, 2H), 7.18 (m, 2H), 6.99 (s, 1H), 3.64 (s, 3H), 1.26 (s, 6H).

### Preparation of 2-(4-(2-chloro-3-fluorophenyl)-4H-1,2,4-triazol-3-yl)-5-(trifluoromethyl)-1H-benzo[d]imidazole (59) Method 11

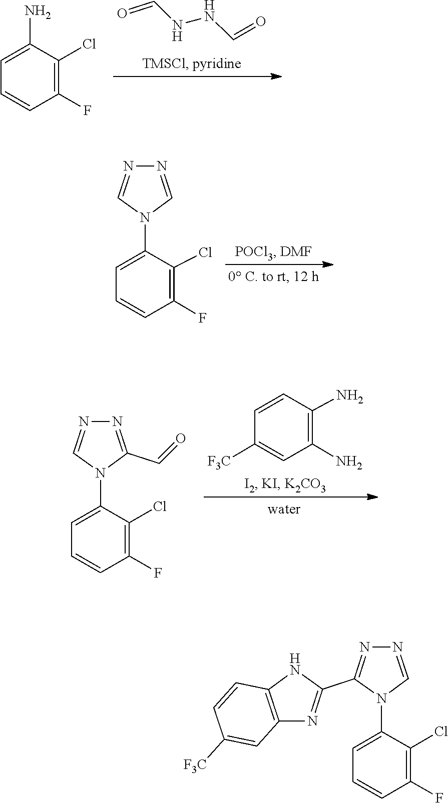

### Step 1. 4-(2-chloro-3-fluorophenyl)-4H-1,2,4-triazole

To a solution of 2-chloro-3-fluoro-aniline (0.50 g, 3.44 mmol) and N-formamidoformamide (0.91 g, 10.3 mmol) in pyridine (30 mL), was added chloro(trimethyl)silane (5598 mg, 51.5 mmol) and stirred at 110° C. for 12 h. LCMS indicated the reaction was completed. Then the reaction mixture was cooled, filtered and concentrated to dryness. The crude was purified flash chromatography (Silica gel column, 30 g, MeOH/EtOAc, 0 to 20%) to give the product 4-(2-chloro-3-fluoro-phenyl)-1,2,4-triazole (420 mg, 2.13 mmol, 61.9% yield) as a white solid. MS (ESI) m/z 198 [M+H]⁺

### Step 2. 4-(2-chloro-3-fluorophenyl)-4H-1,2,4-triazole-3-carbaldehyde

To a solution of 4-(2-chloro-3-fluoro-phenyl)-1,2,4-triazole (260 mg, 1.32 mmol) in DMF (0.5 mL) was added dropwise the POCl₃ (0.61 mL, 6.58 mmol) dissolved in DMF (1.9 mL) at 0° C. under N₂. The reaction mixture was stirred at 0° C. for 1 h. The mixture was then stirred at r.t. for 12 h. Once the reaction was completed, the reaction mixture was quenched by NaHCO₃ aq. (30 mL) and extracted with EtOAc (20 mL×3). The organic layers were combined, dried (Na₂SO₄) and concentrated to give the crude product 4-(2-chloro-3-fluorophenyl)-4H-1,2,4-triazole-3-carbaldehyde (200 mg, 0.886 mmol, 67.4% yield) as a white solid. MS (ESI) m/z 226 [M+H]⁺.

### Step 3. 2-(4-(2-chloro-3-fluorophenyl)-4H-1,2,4-triazol-3-yl)-5-(trifluoromethyl)-1H-benzo[d]imidazole

A solution of 4-(2-chloro-3-fluoro-phenyl)-1,2,4-triazole-3-carbaldehyde (200 mg, 0.886 mmol) and 4-(trifluoromethyl)benzene-1,2-diamine (156 mg, 0.886 mmol) in water (10 mL) was stirred at r.t. for 20 min. To this mixture K₂CO₃ (184 mg, 1.33 mmol) was added and stirred for another 10 min. KI (37 mg, 0.222 mmol) and I₂ (225 mg, 0.886 mmol) were then added. The mixture was then stirred at 90° C. for 2 h. The reaction mixture as quenched by sodium thiosulfate solution (10 mL; 5%). The reaction mixture was extracted with EtOAc (15 mL×3). The organic layers were combined, dried over anhydrous sodium sulfate and concentrated under reduced pressure to give the crude product. The crude was purified by prep-HPLC to give 2-(4-(2-chloro-3-fluorophenyl)-4H-1,2,4-triazol-3-yl)-5-(trifluoromethyl)-1H-benzo[d]imidazole (1 mg, 0.0025 mmol, 0.6% yield) as a brown solid.

MS (ESI) m/z 382 [M+H]⁺.

¹H NMR (400 MHz, Methanol-d₄) δ 8.88 (s, 1H), 7.84 (s, 1H), 7.69 (d, J=8.4 Hz, 1H), 7.63-7.45 (m, 4H).

Examples (Compounds) 69 was synthesized using a method similar to that used in Example 59

| Example | | | | |
|---|---|---|---|---|
| No. | | | | |
| (Compound | | Appearance | | |
| No.) | | and | | MS (m/z) |
| Method | Structure and Name | Yield | ¹H NMR Data | [M + H]⁺ |
| Example 69 Method 11 | 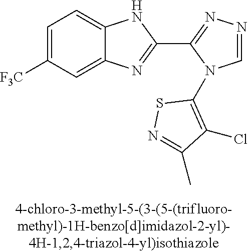 [SMILES: Cc1nsc(-n2cnnc2-c2nc3cc(C(F)(F)F)ccc3[nH]2)c1Cl] | gray solid, yield: 5.7% | ¹H NMR (400 MHz, Methanol-d₄) δ 8.94 (s, 1H), 7.91 (s, 1H), 7.75 (d, J = 8.5 Hz, 1H), 7.57 (d, J = 8.7 Hz, 1H), 2.55 (s, 3H). | 385 |

### Preparation of 2-(4-(4,5-Dichloro-1-methyl-1H-pyrazol-3-yl)-4H-1,2,4-triazol-3-yl)-5-(trifluoromethyl)-1H-benzo[d]imidazole (53) Method 12

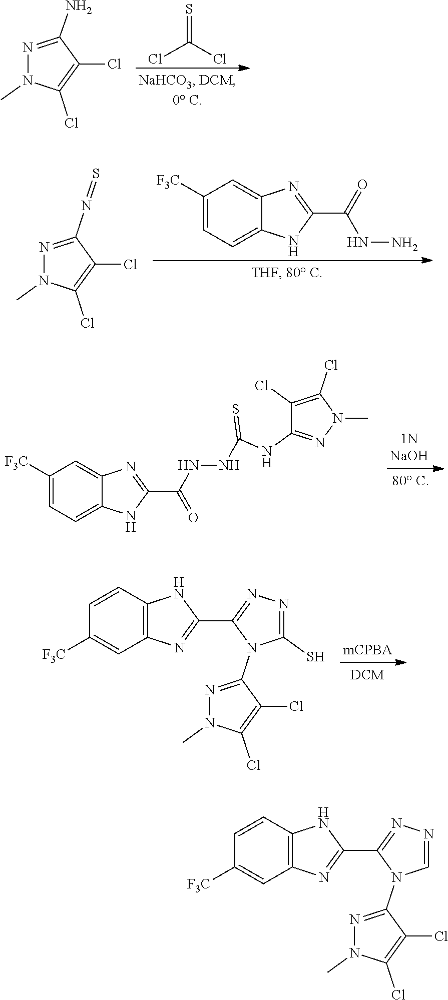

### Step 1. 4,5-Dichloro-3-isothiocyanato-1-methyl-pyrazole

To a mixture of 4,5-dichloro-1-methyl-pyrazol-3-amine (1.00 eq, 500 mg, 3.01 mmol) in DCM (5 mL) and saturated NaHCO₃ (5 mL) was slowly added thiocarbonyl dichloride (1.10 eq, 0.25 mL, 3.31 mmol) at 0° C. The reaction mixture was stirred at 0° C. for 2 h. Once the reaction was completed, the reaction mixture was extracted with DCM and the organic layers were combined, dried over Na₂SO₄, filtered and concentrated under reduced pressure. The residue was used directly in the next step.

### Step 2. N-(4,5-dichloro-1-methyl-1H-pyrazol-3-yl)-2-(5-(trifluoromethyl)-1H-benzo[d]imidazole-2-carbonyl)hydrazine-1-carbothioamide

To a solution of 4,5-dichloro-3-isothiocyanato-1-methyl-pyrazole (1.00 eq, 624 mg, 3.00 mmol) in THF (5 mL) was added 5-(trifluoromethyl)-1H-benzimidazole-2-carbohydrazide (1.00 eq, 733 mg, 3.00 mmol). The reaction mixture was stirred at 70° C. for 2 h. Once the reaction was completed, the organic layer was separated and concentrated. The obtained crude was used directly in the next step.

### Step 3. 4-(4,5-Dichloro-1-methyl-1H-pyrazol-3-yl)-5-(5-(trifluoromethyl)-1H-benzo[d]imidazol-2-yl)-4H-1,2,4-triazole-3-thiol

A suspension of 1-(4,5-dichloro-1-methyl-pyrazol-3-yl)-3-[[5-(trifluoromethyl)-1H-benzimidazole-2-carbonyl]amino]thiourea (1.00 eq, 146 mg, 0.323 mmol) in 1M NaOH (1.00 eq, 4.0 mL, 0.323 mmol) was stirred at 80° C. for 4 h. Once the reaction was completed, 3 M HCl was added to the mixture and adjust the pH to 7. The reaction mixture was extracted with EtOAc. The organic layer was dried, concentrated. The obtained crude was used directly in the next step.

### Step 4. 2-(4-(4,5-Dichloro-1-methyl-1H-pyrazol-3-yl)-4H-1,2,4-triazol-3-yl)-5-(trifluoromethyl)-1H-benzo[d]imidazole

To a solution of 4-(4,5-dichloro-1-methyl-pyrazol-3-yl)-5-[5-(trifluoromethyl)-1H-benzimidazol-2-yl]-1,2,4-triazole-3-thiol (1.00 eq, 30 mg, 0.0691 mmol) in DCM (10 mL) was added mCPBA (2.00 eq, 24 mg, 0.138 mmol) at 0° C. The reaction mixture was stirred at r.t. for 1 h. Once the reaction was completed, the solvent was removed and the residue was purified by prep-HPLC to yield the product (2 mg, 7%).

MS (ESI) m/z 402 [M+H]⁺.

¹H NMR (400 MHz, DMSO-d6) δ 14.09 (bs, 1H), 9.14 (s, 1H), 7.93 (s, 1H), 7.77 (d, J=8.5 Hz, 1H), 7.58 (s, 1H), 3.97 (s, 3H).

Examples (Compounds) 58 was synthesized using a method similar to that used in Example 53

| Example | | | | |
|---|---|---|---|---|
| No. (Compound | | Appearance | | |
| No.) | | and | | MS (m/z) |
| Method | Structure and Name | Yield | ¹H NMR Data | [M + H]⁺ |
| Example 58 Method 12 | 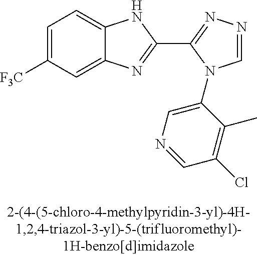 [SMILES: Cc1c(Cl)cncc1-n1cnnc1-c1nc2cc(C(F)(F)F)ccc2[nH]1] | white solid, yield: 17% | ¹H NMR (400 MHz, Methanol-d₄) δ 8.90 (s, 1H), 8.77 (s, 1H), 8.54 (s, 1H), 7.84 (s, 1H), 7.70 (d, J = 8.6 Hz, 1H), 7.54 (d, J = 8.6 Hz, 1H), 2.18 (s, 3H). | 379 |

### Preparation of 2-(4-(2,3-difluorophenyl)-4H-1,2,4-triazol-3-yl)-5-(trifluoromethyl)-1H-benzo[d]imidazole (84) Method 13

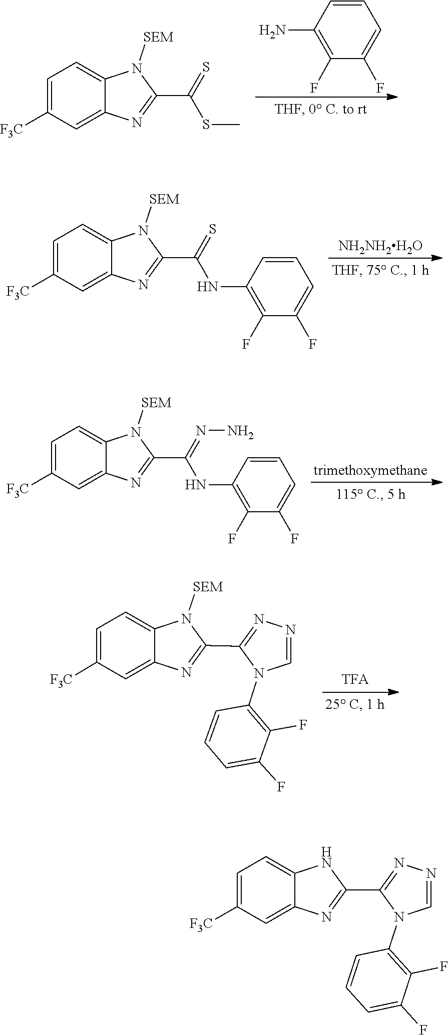

### Step 1. N-(2,3-difluorophenyl)-5-(trifluoromethyl)-1-((2-(trimethylsilyl)ethoxy)methyl)-1H-benzo[d]imidazole-2-carbothioamide

To a solution of 2,3-difluoroaniline (70 mg, 0.541 mmol) in THF (5 mL), was added LiHMDS (1.20 eq, 0.50 mL, 0.590 mmol) at 0° C. under N₂. The mixture was stirred at 0° C. for 0.5 h. Then to the mixture was added methyl 5-(trifluoromethyl)-1-(2-trimethylsilylethoxymethyl)benzimidazole-2-carbodithioate (200 mg, 0.492 mmol). The mixture was stirred at r.t. for 0.5 h. The resulting mixture was concentrated under reduced pressure to give the crude product which was directly used in the next step without further purification. MS (ESI) m/z 488 [M+H]⁺.

### Step 2. N-(2,3-difluorophenyl)-5-(trifluoromethyl)-1-((2-(trimethylsilyl)ethoxy)methyl)-1H-benzo[d]imidazole-2-carbohydrazonamide

To a solution of N-(2,3-difluorophenyl)-5-(trifluoromethyl)-1-(2-trimethylsilylethoxymethyl)benzimidazole-2-carbothioamide (239 mg, 0.490 mmol) in THF (5 mL), was added NH₂NH₂·H₂O (0.12 mL, 1.96 mmol) at r.t. The reaction mixture was stirred at 75° C. for 1 h. Then the mixture was directly concentrated and used for next step without further purification. MS (ESI) m/z 486 [M+H]⁺.

### Step 3. 2-(4-(2,3-difluorophenyl)-4H-1,2,4-triazol-3-yl)-5-(trifluoromethyl)-1-((2-(trimethylsilyl)ethoxy)methyl)-1H-benzo[d]imidazole

A solution of N-(2,3-difluorophenyl)-5-(trifluoromethyl)-1-((2-(trimethylsilyl)ethoxy)methyl)-1H-benzo[d]imidazole-2-carbohydrazonamide (40 mg, 0.0824 mmol) in trimethoxymethane (3.0 mL, 27.4 mmol), was stirred at 110° C. for 5 h. Then the reaction mixture was concentrated and used for next step without further purification. MS (ESI) m/z 496 [M+H]⁺.

### Step 4. 2-(4-(2-chloro-3-fluorophenyl)-4H-1,2,4-triazol-3-yl)-5-(trifluoromethyl)-1H-benzo[d]imidazole

To a solution of 2-(4-(2,3-difluorophenyl)-4H-1,2,4-triazol-3-yl)-5-(trifluoromethyl)-1-((2-(trimethylsilyl)ethoxy)methyl)-1H-benzo[d]imidazole (42 mg, 0.0838 mmol) in DCM (1 mL), was added TFA (1.0 mL, 13.1 mmol) at r.t. The reaction mixture was stirred at 25° C. for 1 h. The reaction mixture was concentrated to dryness. The crude was purified by Prep-HPLC to give the product 2-[4-(2,3-difluorophenyl)-1,2,4-triazol-3-yl]-5-(trifluoromethyl)-1H-benzimidazole (5.0 mg, 0.013 mmol, 15.5% yield) as a white solid.

MS (ESI) m/z 366 [M+H]⁺.

¹H NMR (400 MHz, Methanol-d₄) δ 8.93 (s, 1H), 7.86 (s, 1H), 7.72 (d, J=8.4 Hz, 1H), 7.62-7.50 (m, 2H), 7.48-7.34 (m, 2H).

Examples (Compounds) 52, 62, 63, 66, 67, 70, 71, 85, 91, 99, 105, 106, 114, 115, 118, 119, 120, 123 were synthesized using a method similar to that used in Example 84.

| Example | | | | |
|---|---|---|---|---|
| No. | | | | |
| (Compound | | Appearance | | |
| No.) | | and | | MS (m/z) |
| Method | Structure and Name | Yield | ¹H NMR Data | [M + H]⁺ |
| Example 52 Method 13 | 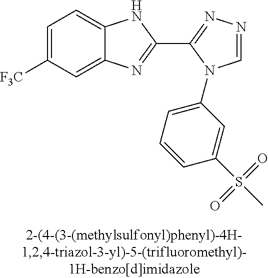 [SMILES: CS(=O)(=O)c1cccc(-n2cnnc2-c2nc3cc(C(F)(F)F)ccc3[nH]2)c1] | white solid, yield: 9.5% | ¹H NMR (400 MHz, DMSO) δ 13.94 (s, 1H), 9.14 (s, 1H), 8.28 (t, J = 1.9 Hz, 1H), 8.10 (dt, J = 7.9, 1.4 Hz, 1H), 8.01 (ddd, J = 8.0, 2.2, 1.1 Hz, 1H), 7.91-7.81 (m, 2H), 7.72 (d, J = 8.5 Hz, 1H), 7.54 (d, J = 8.6 Hz, 1H). | 408 |
| Example 62 Method 13 | 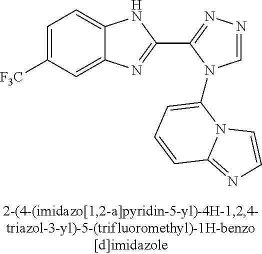 [SMILES: FC(F)(F)c1ccc2[nH]c(-c3nncn3-c3cccc4nccn34)nc2c1] | white solid, yield: 12.3% | ¹H NMR (400 MHz, d6- DMSO) δ 14.03 (bs, 1H), 9.27 (s, 1H), 7.87 (d, J = 9.2 Hz, 1H), 7.67 (s, 1H), 7.64-7.54 (m, 2H), 7.51- 7.42 (m, 3H), 7.32 (d, J = 7.2 Hz, 1H). | 370 |
| Example 63 Method 13 | 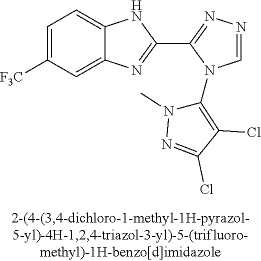 [SMILES: Cn1nc(Cl)c(Cl)c1-n1cnnc1-c1nc2cc(C(F)(F)F)ccc2[nH]1] | white solid, yield: 41.6% | ¹H NMR (400 MHz, d6- DMSO) δ 14.11 (bs, 1H), 9.23 (s, 1H), 7.92 (s, 1H), 7.77 (d, J = 8.4 Hz, 1H), 7.58 (d, J = 8.4 Hz, 1H), 3.69(s, 3H). | 402 |
| Example 66 Method 13 | 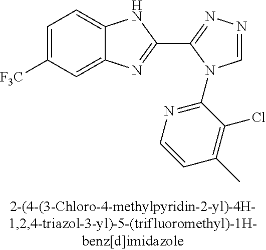 [SMILES: Cc1ccnc(-n2cnnc2-c2nc3cc(C(F)(F)F)ccc3[nH]2)c1Cl] | white solid, yield: 5.6% | ¹H NMR (400 MHz, DMSO-d6) δ 14.08 (s, 1H), 9.19 (s, 1H), 8.47 (d, J = 4.9 Hz, 1H), 7.84- 7.77 (m, 1H), 7.74 (d, J = 4.9 Hz, 1H), 7.69 (d, J = 8.6 Hz, 1H), 7.54 (d, J = 8.6 Hz, 1H). 2.50 (s, 3H). | 379 |
| Example 67 Method 13 | 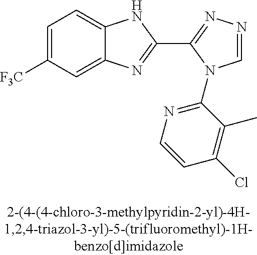 [SMILES: Cc1c(Cl)ccnc1-n1cnnc1-c1nc2cc(C(F)(F)F)ccc2[nH]1] | white solid, yield: 9.6% | ¹H NMR (400 MHz, DMSO) δ 14.10 (s, 1H), 9.16 (s, 1H), 8.42 (d, J = 5.3 Hz, 1H), 7.91-7.74 (m, 2H), 7.70 (d, J = 8.6 Hz, 1H), 7.55 (s, 1H), 2.10 (s, 3H). | 379 |
| Example 70 Method 13 | 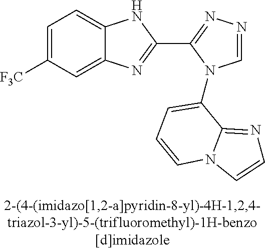 [SMILES: FC(F)(F)c1ccc2[nH]c(-c3nncn3-c3cccn4ccnc34)nc2c1] | white solid, yield: 9.6% | ¹H NMR (400 MHz, DMSO) δ 13.90 (s, 1H), 9.13 (s, 1H), 8.75 (dd, J = 6.8, 1.1 Hz, 1H), 8.08 (d, J = 1.2 Hz, 1H), 7.77 (s, 1H), 7.71-7.61 (m, 2H), 7.50 (d, J = 8.7 Hz, 1H), 7.46 (d, J = 1.2 Hz, 1H), 7.09 (t, J = 7.1 Hz, 1H). | 370 |
| Example 71 Method 13 | 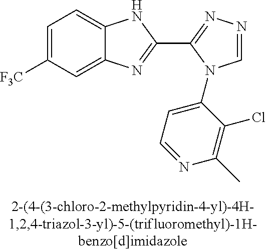 [SMILES: Cc1nccc(-n2cnnc2-c2nc3cc(C(F)(F)F)ccc3[nH]2)c1Cl] | white solid, yield: 3.8% | ¹H NMR (400 MHz, DMSO) δ 14.04 (s, 1H), 9.10 (s, 1H), 8.65 (d, J = 5.1 Hz, 1H), 7.83 (d, J = 1.6 Hz, 1H), 7.74 (d, J = 5.1 Hz, 1H), 7.70 (d, J = 8.5 Hz, 1H), 7.54 (d, J = 8.5 Hz, 1H), 2.67 (s, 3H). | 379 |
| Example 85 Method 13 | 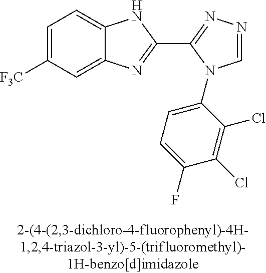 [SMILES: Fc1ccc(-n2cnnc2-c2nc3cc(C(F)(F)F)ccc3[nH]2)c(Cl)c1Cl] | white solid, yield: 5.7% | ¹H NMR (400 MHz, Methanol-d₄) δ 8.87 (s, 1H), 7.86 (s, 1H), 7.74- 7.68 (m, 2H), 7.57-7.47 (m, 2H). | 416 |
| Example 91 Method 13 | 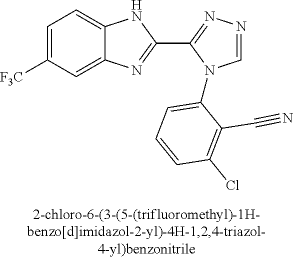 [SMILES: N#Cc1c(Cl)cccc1-n1cnnc1-c1nc2cc(C(F)(F)F)ccc2[nH]1] | white solid, yield: 3.8% | ¹H NMR (400 MHz, DMSO) δ 14.08 (d, J = 10.5 Hz, 1H), 9.10 (d, J = 2.3 Hz, 1H), 7.94 (dd, J = 8.2, 1.5 Hz, 1H), 7.83- 7.44 (m, 5H). | 389 |
| Example 99 Method 13 | 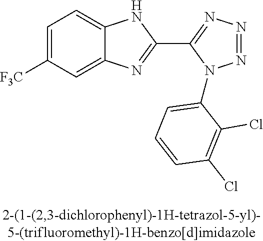 [SMILES: FC(F)(F)c1ccc2[nH]c(-c3nnnn3-c3cccc(Cl)c3Cl)nc2c1] | white solid, yield: 11.8% | ¹H NMR (400 MHz, DMSO-d₆) δ 14.51 (d, J = 18.7 Hz, 1H), 8.06 (dd, J = 8.3, 1.5 Hz, 1H), 8.02-7.84 (m, 2H), 7.80 (t, J = 7.7 Hz, 1H), 7.75- 7.48 (m, 2H). | 399 |
| Example 105 Method 13 | 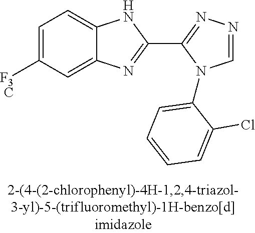 [SMILES: FC(F)(F)c1ccc2[nH]c(-c3nncn3-c3ccccc3Cl)nc2c1] | white solid, yield: 19.6% | ¹H NMR (400 MHz, d6- DMSO) δ 13.86 (bs, 1H), 9.05 (s, 1H), 7.83-7.71 (m, 3H), 7.71-7.61 (m, 2H), 7.61-7.48 (m, 2H). | 364 |
| Example 106 Method 13 | 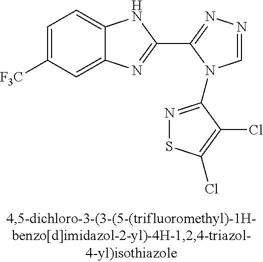 [SMILES: FC(F)(F)c1ccc2[nH]c(-c3nncn3-c3nsc(Cl)c3Cl)nc2c1] | white solid, yield: 9.8% | ¹H NMR (400 MHz, d6- DMSO) δ 14.25 (bs, 1H), 9.25 (s, 1H), 7.95-7.84 (m, 1H), 7.75 (d, J = 8.4 Hz, 1H), 7.67-7.47 (m, 1H). | 405 |
| Example 114 Method 13 | 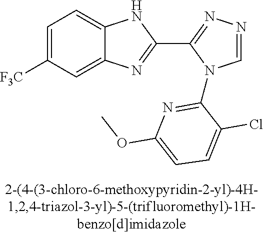 [SMILES: COc1ccc(Cl)c(-n2cnnc2-c2nc3cc(C(F)(F)F)ccc3[nH]2)n1] | white solid, yield: 22.8% | ¹H NMR (400 MHz, Methanol-d₄) δ 9.01 (s, 1H), 7.94 (d, J = 8.8 Hz, 1H), 7.86 (s, 1H), 7.72 (d, J = 8.8 Hz, 1H), 7.56 (d, J = 8.8 Hz, 1H), 7.08 (d, J = 8.8 Hz, 1H), 3.82 (s, 3H). | 395 |
| Example 115 Method 13 | 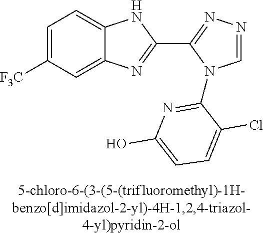 [SMILES: Oc1ccc(Cl)c(-n2cnnc2-c2nc3cc(C(F)(F)F)ccc3[nH]2)n1] | gray solid, yield: 44.3% | ¹H NMR (400 MHz, Methanol-d₄) δ 8.95 (s, 1H), 7.91-7.84 (m, 2H), 7.72 (d, J = 8.8 Hz, 1H), 7.60-7.53 (m, 1H), 6.92 (d, J = 8.8 Hz, 1H). | 381 |
| Example 118 Method 13 | 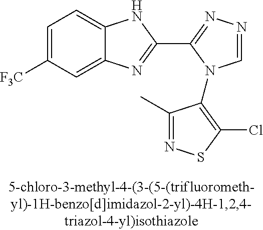 [SMILES: Cc1nsc(Cl)c1-n1cnnc1-c1nc2cc(C(F)(F)F)ccc2[nH]1] | white solid, yield: 10.6% | ¹H NMR (400 MHz, DMSO) δ 14.14 (s, 1H), 9.15 (s, 1H), 7.86 (s, 1H), 7.73 (d, J = 8.5 Hz, 1H), 7.56 (d, J = 8.7 Hz, 1H), 2.23 (s, 3H). | 385 |
| Example 119 Method 13 | 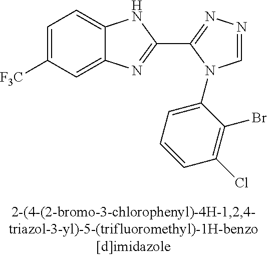 [SMILES: FC(F)(F)c1ccc2[nH]c(-c3nncn3-c3cccc(Cl)c3Br)nc2c1] | white solid, yield: 16.7% | ¹H NMR (400 MHz, DMSO) δ 14.05 (s, 1H), 9.08 (s, 1H), 7.82 (d, J = 1.7 Hz, 1H), 7.73-7.62 (m, 4H), 7.54 (d, J = 8.7 Hz, 1H). | 444 |
| Example 120 Method 13 | 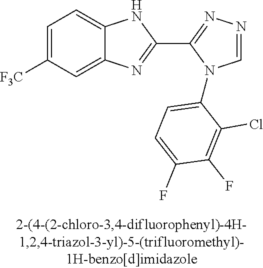 [SMILES: Fc1ccc(-n2cnnc2-c2nc3cc(C(F)(F)F)ccc3[nH]2)c(Cl)c1F] | white solid, yield: 7.6% | ¹H NMR (400 MHz, DMSO) δ 14.07 (s, 1H), 9.07 (s, 1H), 7.86 (d, J = 1.7 Hz, 1H), 7.82-7.68 (m, 3H), 7.55 (d, J = 8.6 Hz, 1H). | 400 |
| Example 123 Method 13 | 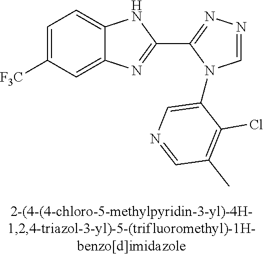 [SMILES: Cc1cncc(-n2cnnc2-c2nc3cc(C(F)(F)F)ccc3[nH]2)c1Cl] | white solid, yield: 31.2% | ¹H NMR (400 MHz, d6- DMSO) δ 14.00 (bs, 1H), 9.09 (s, 1H), 8.77 (s, 1H), 8.76 (s, 1H), 7.80 (s, 1H), 7.68 (d, J = 8.4 Hz, 1H), 7.51 (d, J = 8.4 Hz, 1H), 2.44 (s, 3H). | 379 |

### Preparation of 2-(4-(2,3-dichlorophenyl)-5-methyl-4H-1,2,4-triazol-3-yl)-5-(trifluoromethyl)-1H-benzo[d]imidazole (87)

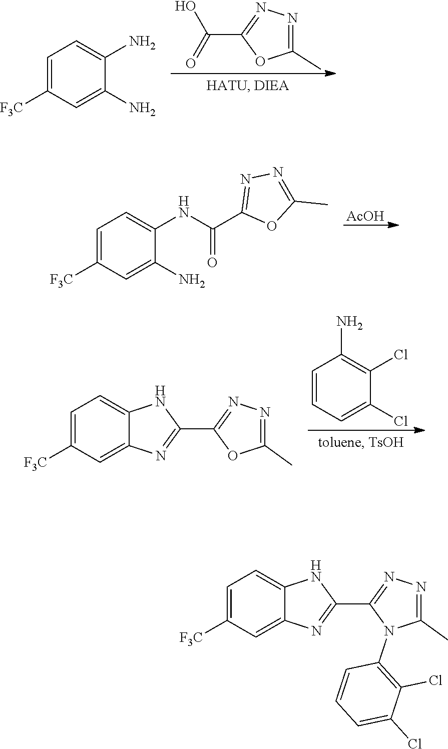

### Step 1. N-(2-amino-4-(trifluoromethyl) phenyl)-5-methyl-1,3,4-oxadiazole-2-carboxamide

To a solution of 4-(trifluoromethyl)benzene-1,2-diamine (2.0 g, 0.01 mol) in DMF (20 mL) was added 5-methyl-1,3,4-oxadiazole-2-carboxylic acid (2.17 g, 0.016 mol), HATU (6.44 g, 0.016 mol) and DIEA (4.38 g, 0.033 mol). The reaction mixture was stirred at 25° C. under N₂ for 12 h. The reaction mixture was diluted with EtOAc and washed with water. The organic layer was separated, dried over anhydrous sodium sulfate and concentrated under vacuum. The residue was purified by silica gel flash column chromatography (PE/EtOAc=2/1) to afford N-(2-amino-4-(trifluoromethyl) phenyl)-5-methyl-1,3,4-oxadiazole-2-carboxamide (1.0 g, 29.20% yield) as a yellow solid. MS (ESI) m/z 287 [M+H]⁺.

### Step 2. 2-methyl-5-(5-(trifluoromethyl)-1H-benzo[d]imidazol-2-yl)-1,3,4-oxadiazole

The solution of N-(2-amino-4-(trifluoromethyl) phenyl)-5-methyl-1,3,4-oxadiazole-2-carboxamide (1.0 g, 3.5 mmol) in AcOH (5 mL) was heated to 80° C. and stirred for 2 h. After the reaction was completed, the resulting mixture was concentrated under reduced pressure to afford 2-methyl-5-(5-(trifluoromethyl)-1H-benzo[d]imidazol-2-yl)-1,3,4-oxadiazole (0.8 g, 82.86% yield) as a white solid. MS (ESI) m/z 269 [M+H]⁺.

### Step 3. 2-(4-(2,3-dichlorophenyl)-5-methyl-4H-1,2,4-triazol-3-yl)-5-(trifluoromethyl)-1H-benzo[d]imidazole

To a solution of 2-methyl-5-(5-(trifluoromethyl)-1H-benzo[d]imidazol-2-yl)-1,3,4-oxadiazole (800 mg, 2.97 mmol) in toluene (10 mL) was added 2,3-dichloroaniline (722 mg, 4.45 mmol) and p-toluenesulfonic acid (767 mg, 4.45 mmol). The reaction mixture was stirred at 110° C. under N₂ for 12 h. The mixture was diluted with EtOAc and washed with water. The organic layer was separated, dried over anhydrous sodium sulfate and concentrated under vacuum. The residue was purified by prep-HPLC to afford 2-(4-(2,3-dichlorophenyl)-5-methyl-4H-1,2,4-triazol-3-yl)-5-(trifluoromethyl)-1H-benzo[d]imidazole (90 mg, 7.0% yield) as a white solid.

MS (ESI) m/z 412 [M+H]⁺.

¹H NMR (400 MHz, DMSO-d₆) δ 13.97 (bs, 1H), 7.94 (dd, J=8.0 Hz, J=1.2 Hz, 1H), 7.81-7.77 (m, 2H), 7.66-7.62 (m, 3H), 2.27 (s, 3H).

### Preparation of 2-(4-(2,3-dichlorophenyl)-5-((methylsulfonyl)methyl)-4H-1,2,4-triazol-3-yl)-5-(trifluoromethyl)-1H-benzo[d]imidazole (88) Method 14

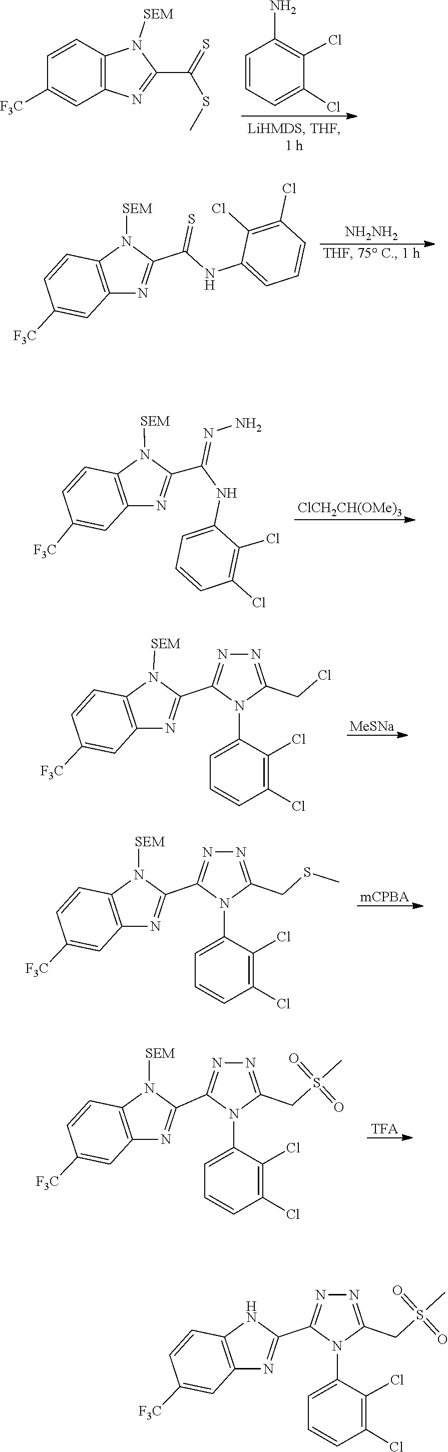

### Step 1. N-(2,3-dichlorophenyl)-5-(trifluoromethyl)-1-((2-(trimethylsilyl)ethoxy) methyl)-1H-benzo[d]imidazole-2-carbothioamide

To a solution of 2,3-dichloroaniline (90 mg, 0.55 mmol) in THF (3 mL) at an ice bath was added dropwise a solution of LiHMDS in THF (1.0 M, 0.8 mL, 0.8 mmol). The mixture was stirred for 5 minutes and a solution of methyl 5-(trifluoromethyl)-1-((2-(trimethylsilyl)ethoxy)methyl)-1H-benzo[d]imidazole-2-carbodithioate (220 mg, 0.55 mmol) in THF (1 mL) was added dropwise. The reaction was stirred for 1 hour. The resulting mixture was diluted with water (10 mL) and EtOAc (20 mL). The organic layer was collected and washed with brine, dried over Na₂SO₄ and concentrated to give the crude product (300 mg) as a yellow oil which was used in the following step without further purification. MS (ESI) m/z 520 [M+H]⁺.

### Step 2. N-(2,3-dichlorophenyl)-5-(trifluoromethyl)-1-((2-(trimethylsilyl) ethoxy)methyl)-1H-benzo[d]imidazole-2-carbohydrazonamide

To a solution of N-(2,3-dichlorophenyl)-5-(trifluoromethyl)-1-((2-(trimethylsilyl)ethoxy)methyl)-1H-benzo[d]imidazole-2-carbothioamide (300 mg) in THF (5 mL) was added hydrazine (80%, 500 mg, 9.88 mmol). The reaction mixture was heated to 75° C. and stirred for 1 hour. The resulting mixture was concentrated to give the crude product (300 mg) as a yellow oil which was used directly in the following step without further purification. MS (ESI) m/z 518 [M+H]⁺.

### Step 3. 2-(5-(chloromethyl)-4-(2,3-dichlorophenyl)-4H-1,2,4-triazol-3-yl)-5-(trifluoromethyl)-1-((2-(trimethylsilyl)ethoxy)methyl)-1H-benzo[d]imidazole

The solution of N-(2,3-dichlorophenyl)-5-(trifluoromethyl)-1-((2-(trimethylsilyl) ethoxy)methyl)-1H-benzo[d]imidazole-2-carbohydrazonamide (300 mg) in 2-chloro-1,1,1-trimethoxyethane (3 mL) was heated to 110° C. and stirred for 4 hours. The resulting mixture was concentrated and the residue was purified through pre-TLC to give the product (100 mg, 31.5% yield over three steps) as a yellow solid. MS (ESI) m/z 576 [M+H]⁺.

### Step 4. 2-(4-(2,3-dichlorophenyl)-5-((methylthio)methyl)-4H-1,2,4-triazol-3-yl)-5-(trifluoromethyl)-1-((2-(trimethylsilyl)ethoxy)methyl)-1H-benzo[d]imidazole

To a solution of 2-(5-(chloromethyl)-4-(2,3-dichlorophenyl)-4H-1,2,4-triazol-3-yl)-5-(trifluoromethyl)-1-((2-(trimethylsilyl)ethoxy)methyl)-1H-benzo[d]imidazole (80 mg, 0.14 mmol) in DMF (2 mL) was added aqueous sodium thiomethoxide (30%, 400 mg, 1.7 mmol). The reaction was stirred for 2 hours. The resulting mixture was diluted with water and EtOAc. The organic layer was washed with brine, dried over Na₂SO₄ and concentrated to give the product (50 mg, 61% yield) as a yellow oil. MS (ESI) m/z 588 [M+H]⁺.

### Step 5. 2-(4-(2,3-dichlorophenyl)-5-((methylsulfonyl)methyl)-4H-1,2,4-triazol-3-yl)-5-(trifluoromethyl)-1-((2-(trimethylsilyl)ethoxy)methyl)-1H-benzo[d]imidazole

To a solution of 2-(4-(2,3-dichlorophenyl)-5-((methylthio)methyl)-4H-1,2,4-triazol-3-yl)-5-(trifluoromethyl)-1-((2-(trimethylsilyl)ethoxy)methyl)-1H-benzo[d]imidazole (50 mg, 0.085 mmol) in DCM (3 mL) was added mCPBA (45 mg, 0.26 mmol). The reaction was stirred for 2 hours. The resulting mixture was diluted with water and EtOAc. The organic layer was washed with aqueous NaHCO₃, dried over Na₂SO₄ and concentrated to give the product (50 mg, 75% yield) as a yellow oil. MS (ESI) m/z 620 [M+H]⁺.

### Step 6. 2-(4-(2,3-dichlorophenyl)-5-((methylsulfonyl)methyl)-4H-1,2,4-triazol-3-yl)-5-(trifluoromethyl)-1H-benzo[d]imidazole

To a solution of 2-(4-(2,3-dichlorophenyl)-5-((methylsulfonyl)methyl)-4H-1,2,4-triazol-3-yl)-5-(trifluoromethyl)-1-((2-(trimethylsilyl)ethoxy)methyl)-1H-benzo[d]imidazole (50 mg, 0.08 mmol) in DCM (2 mL) was added TFA (2 mL). The reaction was stirred for 1 hour. The resulting mixture was concentrated. The residue was purified through pre-HPLC to give the product (15 mg, 37.5% yield) as a white solid.

MS (ESI) m/z 490 [M+H]⁺.

¹H NMR (400 MHz, d6-DMSO) δ 14.08 (bs, 1H), 7.94 (dd, J=8.0 Hz, J=1.6 Hz, 1H), 7.80 (s, 1H), 7.74 (dd, J=8.0 Hz, J=1.6 Hz, 1H), 7.69 (d, J=8.8 Hz, 1H), 7.64 (t, J=8.0 Hz, 1H), 7.54 (d, J=8.0 Hz, 1H), 4.92 (d, J=15.2 Hz, 1H), 4.64 (d, J=15.2 Hz, 1H), 3.19 (s, 3H).

Examples (Compounds) 90, 124, 125, 126, 127 were synthesized using a method similar to that used in Example 88.

| Example | | | | |
|---|---|---|---|---|
| No. | | | | |
| (Compound | | Appearance | | |
| No.) | | and | | MS (m/z) |
| Method | Structure and Name | Yield | ¹H NMR Data | [M + H]⁺ |
| Example 90 Method 14 | 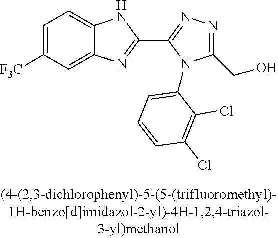 [SMILES: OCc1nnc(-c2nc3cc(C(F)(F)F)ccc3[nH]2)n1-c1cccc(Cl)c1Cl] | white solid, yield: 13% | ¹H NMR (400 MHz, d6-DMSO) δ 14.15- 13.90 (m, 1H), 7.94- 7.88 (dd, J = 8.0 Hz, J = 1.6 Hz, 1H), 7.83- 7.74 (m, 2 H), 7.73- 7.41 (m, 3H), 7.26- 6.92 (m, 1H), 5.53 (t, J = 5.2 Hz, 1H), 4.60- 4.40 (m, 2H). | 428 |
| Example 124 Method 14 | 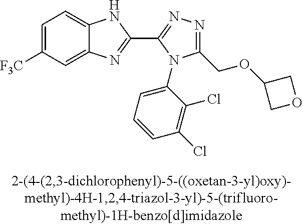 [SMILES: FC(F)(F)c1ccc2[nH]c(-c3nnc(COC4COC4)n3-c3cccc(Cl)c3Cl)nc2c1] | white solid, yield: 17.8% | ¹H NMR (400 MHz, d6-DMSO) δ 14.07 (bs, 1H), 7.95 (dd, J = 8.0 Hz, J = 1.2 Hz, 1H), 7.83 (dd, J = 8.0 Hz, J = 1.2 Hz, 1H), 7.80 (s, 1H), 7.69 (d, J = 8.4 Hz, 1H), 7.64 (t, J = 8.0 Hz, 1H), 7.54 (d, J = 8.4 Hz, 1H), 4.60-4.46 (m, 5H), 4.19-4.11 (m, 1H), 4.10-4.02 (m, 1H). | 484 |
| Example 125 Method 14 | 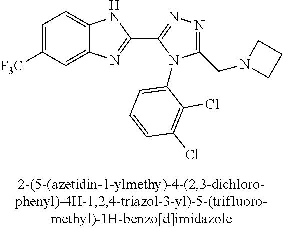 [SMILES: FC(F)(F)c1ccc2[nH]c(-c3nnc(CN4CCC4)n3-c3cccc(Cl)c3Cl)nc2c1] | white solid, yield: 32.6% | ¹H NMR (400 MHz, MeOD-d₄) δ 7.90 (d, J = 8.0 Hz, 1H), 7.79 (s, 1H), 7.69-7.60 (m, 4H), 4.49-4.40 (m, 2H), 4.33-4.29 (m, 4H), 2.57-2.51 (m, 2H). | 467 |
| Example 126 Method 14 | 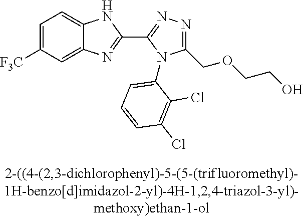 [SMILES: OCCOCc1nnc(-c2nc3cc(C(F)(F)F)ccc3[nH]2)n1-c1cccc(Cl)c1Cl] | white solid, yield: 26.2% | ¹H NMR (400 MHz, MeOD-d₄) δ 7.85- 7.82 (m, 2H), 7.71- 7.67 (m, 2H), 7.58- 7.52 (m, 2H), 4.69 (d, J = 13.2 Hz, 1H), 4.57 (d, J = 13.2 Hz, 1H), 3.57-3.54 (m, 2H), 3.47-3.45 (m, 2H). | 467 |
| Example 127 Method 14 | 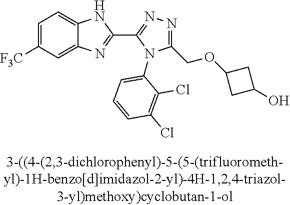 [SMILES: OC1CC(OCc2nnc(-c3nc4cc(C(F)(F)F)ccc4[nH]3)n2-c2cccc(Cl)c2Cl)C1] | white solid, yield: 18% | ¹H NMR (400 MHz, d6-DMSO) δ 14.02 (bs, 1H), 7.93 (dd, J = 8.0 Hz, J =1.6 Hz, 1H), 7.82-7.77 (m, 2H), 7.68 (d, J = 8.4 Hz, 1H), 7.62 (d, J = 8.4 Hz, 1H), 7.52 (d, J = 8.4 Hz, 1H), 4.97 (s, 1H), 4.49-4.35 (m, 2H), 3.68-3.53 (m, 1H), 3.50-3.39 (m, 1H), 2.44-2.31 (m, | 498 |
| | | | 2H), 1.55-1.33 (m, | |
| | | | 2H). | |

### Preparation of 6-[(1-oxo-2-isoquinolyl)methyl]-3H-1,3-benzoxazol-2-one (89)

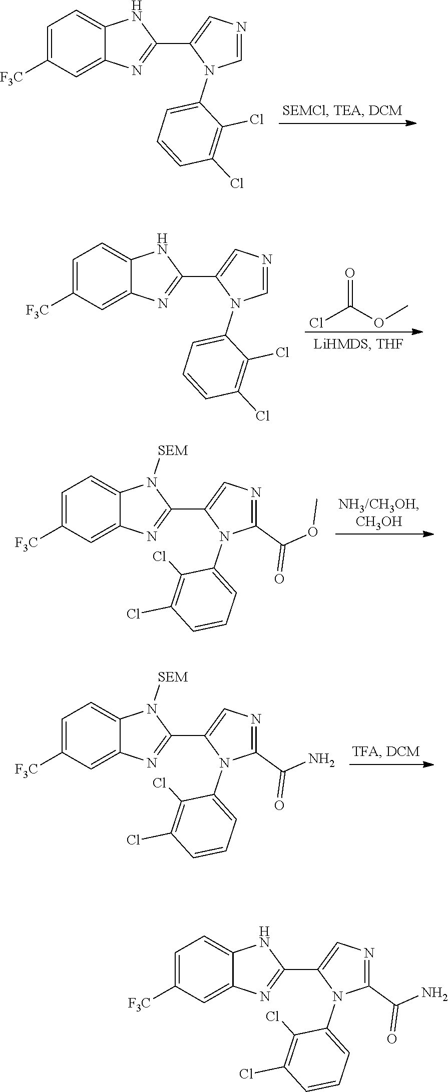

### Step 1. 2-(1-(2,3-dichlorophenyl)-1H-imidazol-5-yl)-5-(trifluoromethyl)-1-((2-(trimethylsilyl)ethoxy)methyl)-1H-benzo[d]imidazole

To a solution of 2-(1-(2,3-dichlorophenyl)-1H-imidazol-5-yl)-5-(trifluoromethyl)-1H-benzo[d]imidazole (500 mg, 1.26 mmol) in DCM (20 mL) was added TEA (382 mg, 3.81 mmol) and SEMCl (252 mg, 1.51 mmol). The mixture was stirred for 2 hours at room temperature. After the reaction was completed, the mixture was diluted with EtOAc and washed with water. The organic layer was separated, dried over anhydrous sodium sulfate and concentrated in vacuo. The residue was purified by silica gel flash column chromatography (PE/EA=1/6) to afford ethyl 1-(2,3-dichlorophenyl)-1H-imidazole-5-carboxylate (300 mg, 45.2% yield) as a yellow solid. MS (ESI) m/z 527 [M+H]⁺.

### Step 2. methyl 1-(2,3-dichlorophenyl)-5-(5-(trifluoromethyl)-1-((2-(trimethylsilyl) ethoxy)methyl)-1H-benzo[d]imidazol-2-yl)-1H-imidazole-2-carboxylate

To a solution of 2-(1-(2,3-dichlorophenyl)-1H-imidazol-5-yl)-5-(trifluoromethyl)-1-((2-(trimethylsilyl)ethoxy)methyl)-1H-benzo[d]imidazole (300 mg, 0.56 mmol) in THF (10 mL) was added LiHMDS (1.1 mL, 1.1 mmol) at an ice bath. The mixture was stirred for 30 minutes, then methyl carbonochloridate (80.5 mg, 0.85 mmol) was added. The reaction mixture was stirred for 1 hour. The reaction mixture was diluted with EtOAc and washed with water. The organic layer was separated, dried over anhydrous sodium sulfate and concentrated in vacuo. The residue was purified by silica gel flash column chromatography (PE/EtOAc=1/8) to afford methyl 1-(2,3-dichlorophenyl)-5-(5-(trifluoromethyl)-1-((2-(trimethylsilyl)ethoxy)methyl)-1H-benzo[d]imidazol-2-yl)-1H-imidazole-2-carboxylate (200 mg, 60.0% yield) as a yellow solid. MS (ESI) m/z 585 [M+H]⁺.

### Step 3. 1-(2,3-dichlorophenyl)-5-(5-(trifluoromethyl)-1-((2-(trimethylsilyl)ethoxy) methyl)-1H-benzo[d]imidazol-2-yl)-1H-imidazole-2-carboxamide

To a solution of methyl 1-(2,3-dichlorophenyl)-5-(5-(trifluoromethyl)-1-((2-(trimethylsilyl)ethoxy)methyl)-1H-benzo[d]imidazol-2-yl)-1H-imidazole-2-carboxylate (200 mg, 0.34 mmol) in NH₃/MeOH (20 mL). The mixture was stirred for 16 hours. After the reaction was completed, the mixture was concentrated to afford 1-(2,3-dichlorophenyl)-5-(5-(trifluoromethyl)-1-((2-(trimethylsilyl)ethoxy)methyl)-1H-benzo[d]imidazol-2-yl)-1H-imidazole-2-carboxamide (200 mg, 100% yield) as a yellow solid which was used in the next step without further purification. MS (ESI) m/z 570 [M+H]⁺.

### Step 4. 1-(2,3-dichlorophenyl)-5-(5-(trifluoromethyl)-1H-benzo[d]imidazol-2-yl)-1H-imidazole-2-carboxamide

To a solution of 1-(2,3-dichlorophenyl)-5-(5-(trifluoromethyl)-1-((2-(trimethylsilyl) ethoxy)methyl)-1H-benzo[d]imidazol-2-yl)-1H-imidazole-2-carboxamide (200 mg, crude) in DCM (10 mL) were added TFA (10 mL) at 0° C. The reaction mixture was stirred for 4 hours. After the reaction was completed, the mixture was diluted with water and adjusted pH to 7. The resulting mixture was diluted with EtOAc and washed with water. The organic layer was separated, dried over anhydrous sodium sulfate and concentrated in vacuo. The residue was purified by prep-HPLC to afford 1-(2,3-dichlorophenyl)-5-(5-(trifluoromethyl)-1H-benzo[d]imidazol-2-yl)-1H-imidazole-2-carboxamide (6.7 mg, 4.3% yield) as a yellow powder.

MS (ESI) m/z 440 [M+H]⁺.

¹H NMR (400 MHz, DMSO-d₆) b 13.34 (bs, 1H), 8.03-7.97 (m, 2H), 7.76 (d, J=8.0 Hz, J=1.6 Hz, 1H), 7.73-7.55 (m, 3H), 7.53 (d, J=8.0 Hz, J=1.6 Hz, 1H), 7.50-7.40 (m, 2H).

### Preparation of 2-(4-(2,3-Dichlorophenyl)-5-(5-(trifluoromethyl)-1H-benzo[d]imidazol-2-yl)-4H-1,2,4-triazol-3-yl)ethan-1-ol (121)

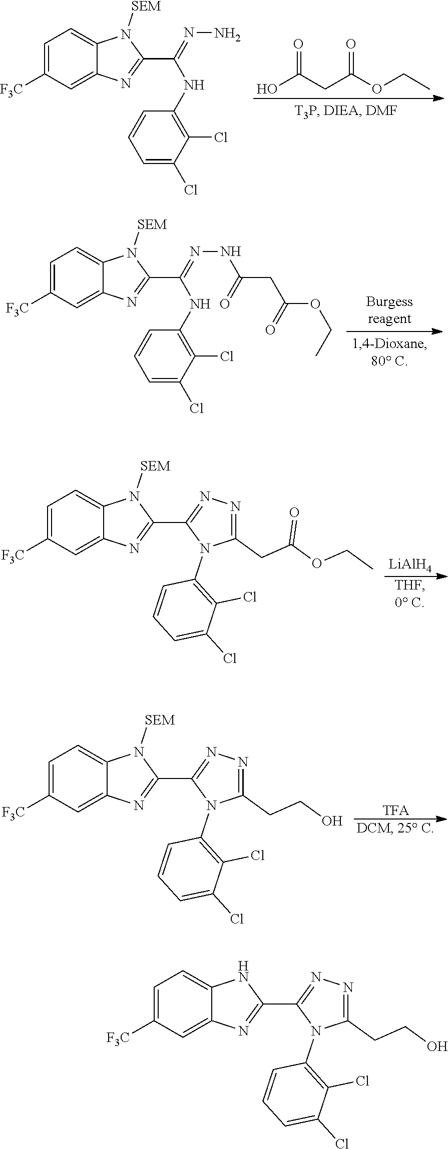

### Step 1. Ethyl 3-(2-(((2,3-dichlorophenyl)amino)(5-(trifluoromethyl)-1-((2-(trimethylsilyl)ethoxy)methyl)-1H-benzo[d]imidazol-2-yl)methylene)hydrazineyl)-3-oxopropanoate

To a solution of 3-ethoxy-3-oxo-propanoic acid (1.00 eq, 0.17 mL, 1.46 mmol) and 3-ethoxy-3-oxo-propanoic acid (1.00 eq, 0.17 mL, 1.46 mmol) in DMF (10 mL) was added DIEA (3.00 eq, 0.76 mL, 4.37 mmol) and T₃P (1.50 eq, 1390 mg, 2.18 mmol). Then the mixture was stirred at 25° C. for overnight. The reaction progress was monitored by LC/MS. Once completed, the mixture was diluted with DCM and washed with water. The organic layer was dried over Na₂SO₄, filtered and concentrated under reduced pressure. The residue was directly used in the next step. MS (ESI) m/z 632 [M+H]⁺.

### Step 2. Ethyl 2-[4-(2,3-dichlorophenyl)-5-[5-(trifluoromethyl)-1-(2-trimethylsilylethoxymethyl)benzimidazol-2-yl]-1,2,4-triazol-3-yl]acetate

To a solution of Ethyl 3-(2-(((2,3-dichlorophenyl)amino)(5-(trifluoromethyl)-1-((2-(trimethylsilyl)ethoxy)methyl)-1H-benzo[d]imidazol-2-yl)methylene)hydrazineyl)-3-oxopropanoate (1.00 eq, 470 mg, 0.743 mmol) in 1,4-dioxane (10 mL) was added burgess reagent (3.00 eq, 531 mg, 2.23 mmol) and the mixture was stirred at 80° C. for 1 h. The reaction progress was monitored by LC/MS. Once completed, the solvent was removed under reduced pressure and the residue was purified by column chromatograph (PE/EA=1/1) to yield the product (180 mg, 39%). MS (ESI) m/z 614 [M+H]⁺.

### Step 3. 2-[4-(2,3-Dichlorophenyl)-5-[5-(trifluoromethyl)-1-(2-trimethylsilylethoxymethyl)benzimidazol-2-yl]-1,2,4-triazol-3-yl]ethanol

To a solution of ethyl 2-[4-(2,3-dichlorophenyl)-5-[5-(trifluoromethyl)-1-(2-trimethylsilylethoxymethyl)benzimidazol-2-yl]-1,2,4-triazol-3-yl]acetate (1.00 eq, 107 mg, 0.174 mmol) in THF (2 mL) was added LiAlH₄ (1.00 eq, 6.6 mg, 0.174 mmol) and the mixture was stirred at 0° C. for 1 h. The reaction progress was monitored by LC/MS. Once completed, to the mixture was added 6.6 ul water, 6.6 ul 15% NaOH and 13 ul water. The reaction was dried over Na₂SO₄, filtered and concentrated under reduced pressure. The crude product was used directly in the next step. MS (ESI) m/z 572 [M+H]⁺.

### Step 4. 2-[4-(2,3-Dichlorophenyl)-5-[5-(trifluoromethyl)-1H-benzimidazol-2-yl]-1,2,4-triazol-3-yl]ethanol

To a solution of 2-[4-(2,3-dichlorophenyl)-5-[5-(trifluoromethyl)-1-(2-trimethylsilylethoxymethyl)benzimidazol-2-yl]-1,2,4-triazol-3-yl]ethanol (1.00 eq, 100 mg, 0.174 mmol) in DCM (2 mL) was added TFA (1 mL) and the mixture was stirred at 25° C. for 1 h. The reaction progress was monitored by LC/MS. Once completed, the solvent was removed and the residue was purified by prep-HPLC to yield the desired product (2 mg, 3%).

MS (ESI) m/z 442 [M+H]⁺.

¹H NMR (400 MHz, DMSO-d6) δ 13.96 (bs, 1H), 7.95 (dd, J=8.2, 1.5 Hz, 1H), 7.80-7.74 (m, 2H), 7.69-7.61 (m, 2H), 7.52 (s, 1H), 4.82 (t, J=5.6 Hz, 1H), 3.70 (tt, J=11.7, 5.8 Hz, 2H), 2.80-2.64 (m, 2H).

### Preparation of 2-[4-(2,3-Dichlorophenyl)-5-[5-(trifluoromethyl)-1H-benzimidazol-2-yl]-1,2,4-triazol-3-yl]acetamide (122)

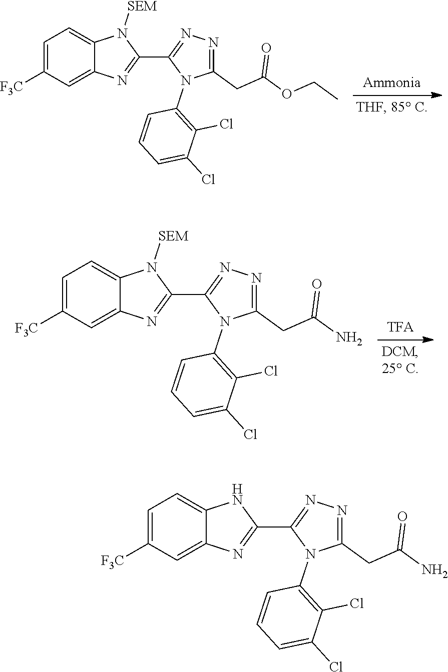 [SMILES: CCOC(=O)Cc1nnc(-c2nc3cc(C(F)(F)F)ccc3n2COCC[Si](C)(C)C)n1-c1cccc(Cl)c1Cl ; C[Si](C)(C)CCOCn1c(-c2nnc(CC(N)=O)n2-c2cccc(Cl)c2Cl)nc2cc(C(F)(F)F)ccc21 ; NC(=O)Cc1nnc(-c2nc3cc(C(F)(F)F)ccc3[nH]2)n1-c1cccc(Cl)c1Cl]

### Step 1. 2-[4-(2,3-Dichlorophenyl)-5-[5-(trifluoromethyl)-1-(2-trimethylsilylethoxymethyl)benzimidazol-2-yl]-1,2,4-triazol-3-yl]acetamide

To a solution of ethyl 2-[4-(2,3-dichlorophenyl)-5-[5-(trifluoromethyl)-1-(2-trimethylsilylethoxymethyl)benzimidazol-2-yl]-1,2,4-triazol-3-yl]acetate (1.00 eq, 155 mg, 0.253 mmol) in ethanol (1 mL) was added ammonia (25%, 1 mL) and the mixture was stirred at 80° C. for 2 h. The reaction progress was monitored by LC/MS. Once completed, the solvent was removed and the residue was used directly in the next step. MS (ESI) m/z 585 [M+H]⁺.

### Step 2.2-[4-(2,3-Dichlorophenyl)-5-[5-(trifluoromethyl)-1H-benzimidazol-2-yl]-1,2,4-triazol-3-yl]acetamide

To a solution of 2-[4-(2,3-dichlorophenyl)-5-[5-(trifluoromethyl)-1-(2-trimethylsilylethoxymethyl)benzimidazol-2-yl]-1,2,4-triazol-3-yl]acetamide (1.00 eq, 148 mg, 0.253 mmol) in DCM (1 mL) was added TFA (1.0 mL) and the mixture was stirred at 25° C. for 2 h. The reaction progress was monitored by LC/MS. Once completed, the solvent was removed and the residue was purified by prep-HPLC to yield the product (4 mg, 3%). MS (ESI) m/z 455 [M+H]⁺. ¹H NMR (400 MHz, DMSO-d₆) δ 14.02 (bs, 1H), 7.92 (dd, J=8.1, 1.6 Hz, 1H), 7.78 (s, 1H), 7.67 (dd, J=8.0, 1.6 Hz, 1H), 7.60 (t, J=8.0 Hz, 1H), 7.45 (s, 1H), 7.04 (s, 1H), 3.79 (d, J=16.5 Hz, 1H), 3.46 (d, J=16.5 Hz, 1H).

### Preparation of 4-(2,3-dichlorophenyl)-5-(5-(trifluoromethyl)-1H-benzo[d]imidazol-2-yl)-4H-1,2,4-triazole-3-thiol (108), 2-(4-(2,3-dichlorophenyl)-5-(methylthio)-4H-1,2,4-triazol-3-yl)-5-(trifluoromethyl)-1H-benzo[d]imidazole (109) and 2-(4-(2,3-dichlorophenyl)-5-(oxetan-3-yloxy)-4H-1,2,4-triazol-3-yl)-5-(trifluoromethyl)-1H-benzo[d]imidazole (117) Method 15

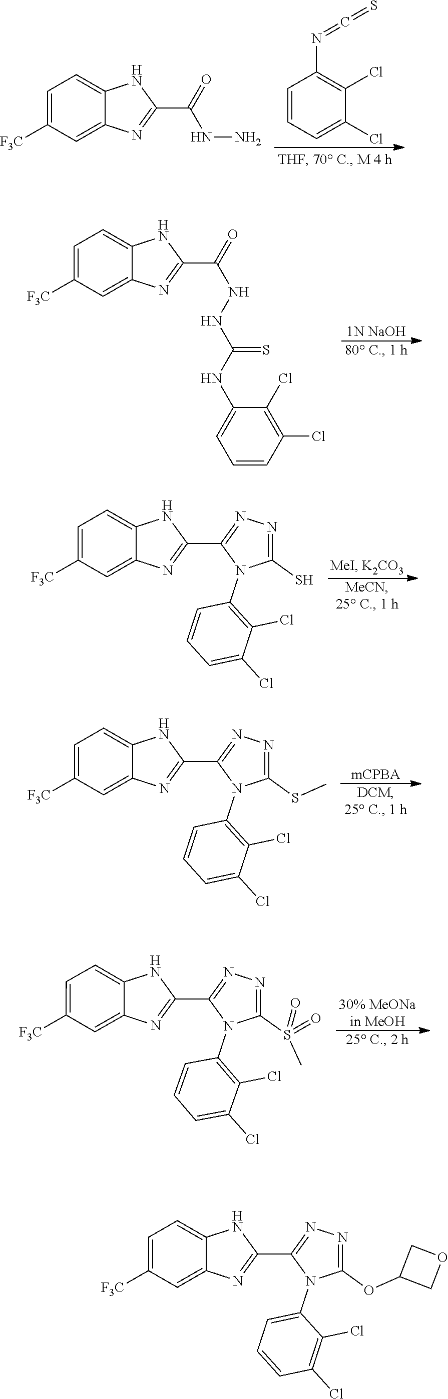

### Step 1. N-(2,3-dichlorophenyl)-2-(5-(trifluoromethyl)-1H-benzo[d]imidazole-2-carbonyl)hydrazine-1-carbothioamide

To a solution of 5-(trifluoromethyl)-1H-benzimidazole-2-carbohydrazide (1000 mg, 4.10 mmol) in THF (5 mL), was added 1,2-dichloro-3-isothiocyanato-benzene (919 mg, 4.51 mmol) and the was stirred at 80° C. for 4 h. The reaction progress was monitored by LCMS. Once completed, the organic layer was separated and concentrated to give the crude product N-(2,3-dichlorophenyl)-2-(5-(trifluoromethyl)-1H-benzo[d]imidazole-2-carbonyl)hydrazine-1-carbothioamide (1700 mg, 3.79 mmol, 92.6% yield) as a brown solid and used directly in the next step. MS (ESI) m/z 448 [M+H]⁺

### Step 2. 4-(2,3-dichlorophenyl)-5-(5-(trifluoromethyl)-1H-benzo[d]imidazol-2-yl)-4H-1,2,4-triazole-3-thiol

A suspension of 1-(2,3-dichlorophenyl)-3-[[5-(trifluoromethyl)-1H-benzimidazole-2-carbonyl]amino]thiourea (3671 mg, 8.19 mmol) in 2N NaOH (100 mL) was stirred at 100° C. for 1 h. The reaction mixture was stirred at 100° C. for 2 h. 1 M HCl (2 mL) was added to the reaction mixture and adjust the pH to 7. The reaction mixture was extracted with EtOAc (10 mL×3). The organic layer was dried, concentrated. The obtained crude was used directly in the next step. MS (ESI) m/z 430 [M+H]⁺.

### Step 3. 2-(4-(2,3-dichlorophenyl)-5-(methylthio)-4H-1,2,4-triazol-3-yl)-5-(trifluoromethyl)-1H-benzo[d]imidazole

To a solution of N-(2,3-dichlorophenyl)-2-(5-(trifluoromethyl)-1H-benzo[d]imidazole-2-carbonyl)hydrazine-1-carbothioamide (380 mg, 0.883 mmol) in K₂CO₃ (244 mg, 1.77 mmol) was added iodomethane (0.082 mL, 1.32 mmol) at room temperature and stirred at 25° C. for 1 h. The reaction mixture was filtrated and concentrated. The obtained crude was used directly in the next step. MS (ESI) m/z 444 [M+H]⁺.

### Step 4. 2-(4-(2,3-dichlorophenyl)-5-(methylsulfonyl)-4H-1,2,4-triazol-3-yl)-5-(trifluoromethyl)-1H-benzo[d]imidazole

To a solution of 2-(4-(2,3-dichlorophenyl)-5-(methylthio)-4H-1,2,4-triazol-3-yl)-5-(trifluoromethyl)-1H-benzo[d]imidazole (250 mg, 0.563 mmol) in DCM (5 mL), was added mCPBA (388 mg, 2.25 mmol) at room temperature under N₂. The reaction mixture was stirred at 25° C. for 2 h. Na₂S₂O₃ aq. (10 mL) and NaHCO₃ (10 mL) were added to quench this reaction. The reaction mixture was extracted with EtOAc (20 mL×3). The organics were then combined and dried (Na₂SO₄) before concentration to dryness to give the product 2-(4-(2,3-dichlorophenyl)-5-(methylsulfonyl)-4H-1,2,4-triazol-3-yl)-5-(trifluoromethyl)-1H-benzo[d]imidazole (150 mg, 0.32 mmol, 56% yield) a brown solid. MS (ESI) m/z 476 [M+H]⁺.

### Step 5. 2-(4-(2,3-dichlorophenyl)-5-(oxetan-3-yloxy)-4H-1,2,4-triazol-3-yl)-5-(trifluoromethyl)-1H-benzo[d]imidazole

To a solution of 2-(4-(2,3-dichlorophenyl)-5-(methylsulfonyl)-4H-1,2,4-triazol-3-yl)-5-(trifluoromethyl)-1H-benzo[d]imidazole (50 mg, 0.105 mmol) and oxetan-3-ol (19 mg, 0.262 mmol) in DMF (2 mL), was added NaH (10 mg, 0.262 mmol) at room temperature under N₂. The reaction mixture was stirred at 25° C. for 2 h. Then the reaction mixture was added H₂O (20 mL) and extracted with EtOAc (20 mL×3). The organics layers were combined, dried (Na₂SO₄) and concentrated in vacuo. The crude was purification with Prep-HPLC to give the product 2-(4-(2,3-dichlorophenyl)-5-(oxetan-3-yloxy)-4H-1,2,4-triazol-3-yl)-5-(trifluoromethyl)-1H-benzo[d]imidazole (10 mg, 0.02 mmol, 19.9% yield) as a white solid.

MS (ESI) m/z 470 [M+H]⁺.

¹H NMR (400 MHz, Methanol-d₄) δ 7.98-7.86 (m, 2H), 7.77-7.22 (m, 2H), 7.70-7.56 (m, 2H), 5.91-5.84 (m, 1H), 5.14-5.06 (m, 2H), 4.83-4.73 (m, 2H).

Examples (Compounds) 131 was synthesized using a method similar to that used in Example 117.

| Example | | | | |
|---|---|---|---|---|
| No. | | | | |
| (Compound | | Appearance | | |
| No.) | | and | | MS (m/z) |
| Method | Structure and Name | Yield | ¹H NMR Data | [M + H]⁺ |
| Example 131 Method 15 | 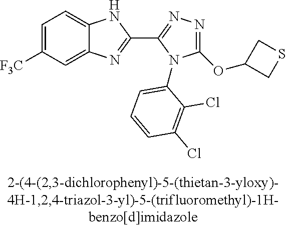 [SMILES: FC(F)(F)c1ccc2[nH]c(-c3nnc(OC4CSC4)n3-c3cccc(Cl)c3Cl)nc2c1] | white solid, yield: 3.8% | ¹H NMR (400 MHz, Methanol-d₄) δ 7.89- 7.71 (m, 2H), 7.67 (d, J = 8.4 Hz, 1H), 7.60-7.43 (m, 3H), 5.74 (p, J = 8.8 Hz, 1H), 4.16 (q, J = 9.1 Hz, 2H), 3.47-3.39 (m, 2H) | 486 |

### Preparation of methyl 2-(2-(4-(2,3-dichlorophenyl)-4H-1,2,4-triazol-3-yl)-1H-benzo[d]imidazol-5-yl)propanoate (101) and 2-(2-(4-(2,3-dichlorophenyl)-4H-1,2,4-triazol-3-yl)-1H-benzo[d]imidazol-5-yl)propan-1-ol (104) Method 16

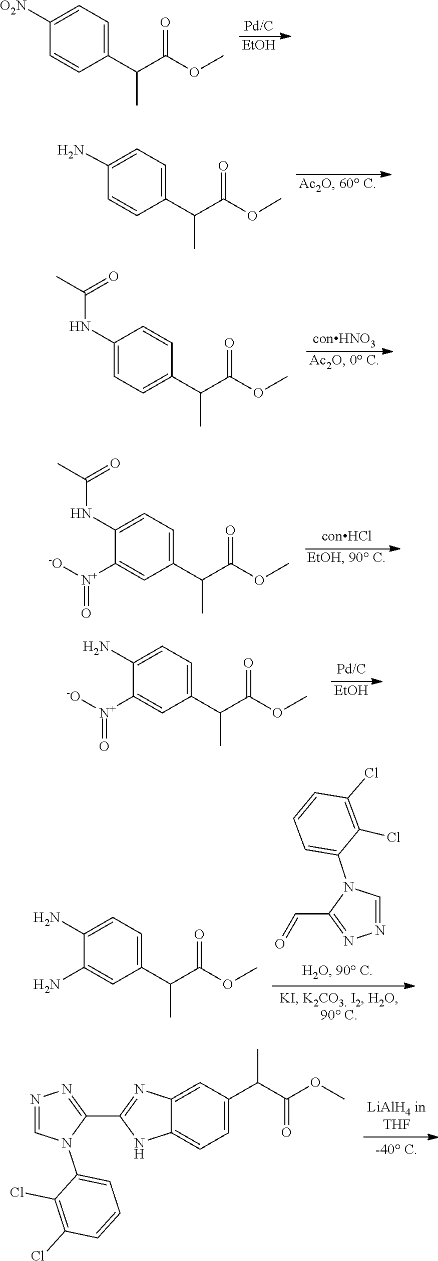

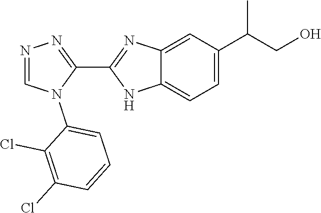 [SMILES: CC(CO)c1ccc2[nH]c(-c3nncn3-c3cccc(Cl)c3Cl)nc2c1]

### Step 1. methyl 2-(4-aminophenyl)propanoate

To a solution of methyl 2-(4-nitrophenyl)propanoate (1.00 eq, 5.00 g, 23.9 mmol) in Ethanol (100 mL) was added Pd/C (0.0100 eq, 253 mg, 0.239 mmol). Then the reaction mixture was stirred overnight at room temperature under an atmosphere of hydrogen (balloon). The reaction solution was filtered and the filtrate was concentrated to afford methyl 2-(4-aminophenyl)propanoate (3.50 g, 19.5 mmol, 81.71% yield) as a yellow oil. The crude product was directly used for the next step without further purification. MS (ESI) m/z 180.1 [M+H]⁺.

### Step 2. 3-(4-methoxybenzyl)-6-methylbenzo[d]oxazol-2 (3H)-one

A solution of methyl 2-(4-aminophenyl)propanoate (1.00 eq, 1.5 g, 8.37 mmol) in Ac₂O (20 mL) was heated at 60° C. for 1 h. The reaction solution was poured into H₂O. The crude product was extracted with EtOAc (20 mL×3). The combined organic layers were washed with water (50 mL×3), dried over anhydrous sodium sulfate, filtered, concentrated to afford crude methyl 2-(4-acetamidophenyl)propanoate (1.80 g, 8.14 mmol, 97.20% yield) as a yellow oil. The crude was used directly at next step. MS (EST) m/z 222.1 [M+H]⁺.

### Step 3. methyl 2-(4-acetamido-3-nitrophenyl)propanoate

A solution of methyl 2-(4-acetamidophenyl)propanoate (1.00 eq, 1.80 g, 8.14 mmol) in Ac₂O (15 mL) was cooled to 0° C. con. HNO₃ (1 mL, 14 mmol) was added and the mixture. The reaction mixture was stirred at r.t. for 2 h. The yellow solution was poured in ice. The aqueous layer was extracted with DCM, washed with saturated aqueous NaHCO₃ solution, dried over Na₂SO₄ and evaporated to afford methyl 2-(4-acetamido-3-nitro-phenyl)propanoate (1.10 g, 4.13 mmol, 50.78% yield) as a yellow solid. (ESI) m/z 267.1 [M+H]⁺.

### Step 4. methyl 2-(4-amino-3-nitrophenyl)propanoate

To a solution of methyl 2-(4-acetamido-3-nitro-phenyl)propanoate (1.00 eq, 1.10 g, 4.13 mmol) in Ethanol (20 mL) was added con·HCl (2.0 mL). The reaction solution was stirred at 90° C. for 5 h. After the reaction mixture was cooled to r.t., The solution was concentrated under vacuum to afford methyl 2-(4-amino-3-nitrophenyl)propanoate (720 mg, 3.43 mmol, 82.91% yield) as a yellow solid. MS (ESI) m/z 225.2 [M+H]⁺.

### Step 5. methyl 2-(3,4-diaminophenyl)propanoate

To a solution of methyl 2-(4-amino-3-nitro-phenyl)propanoate (1.00 eq, 720 mg, 3.21 mmol) in Ethanol (20 mL) was added 10% Pd/C (0.0500 eq, 170 mg, 0.161 mmol). Then the mixture was stirred overnight at room temperature under an atmosphere of hydrogen (balloon). The reaction solution was filtered and the filtrate was concentrated. Purified by flash (C18) to afford methyl 2-(3,4-diaminophenyl)propanoate (460 mg, 2.37 mmol, 73.75% yield) as a yellow oil. MS (ESI) m/z 195.1 [M+H]⁺.

### Step 6. methyl 2-(2-(4-(2,3-dichlorophenyl)-4H-1,2,4-triazol-3-yl)-1H-benzo[d]imidazol-5-yl)propanoate

A mixture of 4-methylsulfonylbenzene-1,2-diamine (1.20 eq, 185 mg, 0.991 mmol) and 4-(2,3-dichlorophenyl)-1,2,4-triazole-3-carbaldehyde (1.00 eq, 200 mg, 0.826 mmol) in Water (15 mL) was stirred 90° C. for 2 h. After cooling the reaction mixture to r.t., K₂CO₃ (3.00 eq, 343 mg, 2.48 mmol), KI (0.500 eq, 69 mg, 0.413 mmol), I₂ (2.00 eq, 419 mg, 1.65 mmol) were added to the mixture. The reaction mixture was then stirred at 90° C. for 30 mins. After cooling to r.t. The mixture was extracted with DCM (20 mL×3), the organic layers were combined, washed with water, dried over Na₂SO₄ and evaporated. The residue was purified by flash (C18) to afford methyl 2-[2-[4-(2,3-dichlorophenyl)-1,2,4-triazol-3-yl]-1H-benzimidazol-5-yl]propanoate (210 mg, 0.504 mmol, 24.50% yield) as a white solid.

(ESI) m/z 416.2 [M+H]⁺.

¹H NMR (400 MHz, DMSO-d₆) δ 13.53 (s, 1H), 9.02 (s, 1H), 7.91 (dd, J=8.2, 1.5 Hz, 1H), 7.77 (dd, J=8.0, 1.5 Hz, 1H), 7.59 (t, J=8.1 Hz, 1H), 7.43 (d, J=8.4 Hz, 1H), 7.37 (s, 1H), 7.12 (s, 1H), 3.92-3.85 (m, 1H), 3.56 (s, 3H), 1.40 (d, J=7.1 Hz, 3H).

### Step 7. 2-(2-(4-(2,3-dichlorophenyl)-4H-1,2,4-triazol-3-yl)-1H-benzo[d]imidazol-5-yl)propan-1-ol

To a solution of methyl 2-[2-[4-(2,3-dichlorophenyl)-1,2,4-triazol-3-yl]-1H-benzimidazol-5-yl]propanoate (1.00 eq, 40 mg, 0.0961 mmol) in THF (10 mL) was added LiAlH₄ in THF (5.00 eq, mg, 0.480 mmol) at −40° C. Then the mixture was stirred at −40° C. for 3 h. The reaction was then quenched by the addition of 1 mL of NH₄Cl solution and H₂O (10 mL). The reaction mixture was extracted with DCM (10 mL×3), the organic layers were combined and washed with water, dried over Na₂SO₄ and evaporated. The residue was purified by prep-HPLC to afford 2-[2-[4-(2,3-dichlorophenyl)-1,2,4-triazol-3-yl]-1H-benzimidazol-5-yl]propan-1-ol (10 mg, 0.0258 mmol, 26.80% yield) as a white solid.

(ESI) m/z 388.2 [M+H]⁺.

1H NMR (400 MHz, DMSO-d6) δ 13.32 (s, 1H), 9.00 (s, 1H), 7.91 (d, J=8.1 Hz, 1H), 7.76 (dd, J=8.0, 1.5 Hz, 1H), 7.59 (t, J=8.1 Hz, 1H), 7.46-7.22 (m, 2H), 7.20-6.99 (m, 1H), 4.79-4.47 (m, 1H), 3.56-3.39 (m, 2H), 3.00-2.77 (m, 1H), 1.26-1.15 (m, 3H).

Examples (Compounds) 102, 103, 107, 110, 111, 112, 113, 129, 130, 133, 134, 135 were synthesized using a method similar to that used in Example 104.

| Example | | | | |
|---|---|---|---|---|
| No. | | | | |
| (Compound | | Appearance | | |
| No.) | | and | | MS (m/z) |
| Method | Structure and Name | Yield | ¹H NMR Data | [M + H]⁺ |
| Example 102 Method 16 | 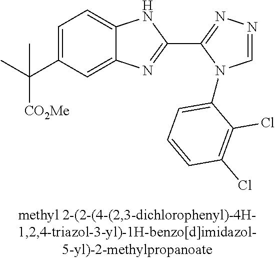 [SMILES: COC(=O)C(C)(C)c1ccc2[nH]c(-c3nncn3-c3cccc(Cl)c3Cl)nc2c1] | white solid, yield: 23.5% | ¹H NMR (400 MHz, DMSO-d₆) δ 13.50 (d, J = 16.5 Hz, 1H), 9.02 (s, 1H), 7.96-7.87 (m, 1H), 7.81-7.73 (m, 1H), 7.65-7.55 (m, 1H), 7.50-7.31 (m, 2H), 7.23-7.07 (m, 1H), 3.56 (d, J = 9.7 Hz, 3H), 1.53 (d, J = 13.8 Hz, 6H). | 430 |
| Example 103 Method 16 | 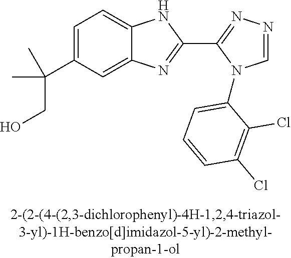 [SMILES: CC(C)(CO)c1ccc2[nH]c(-c3nncn3-c3cccc(Cl)c3Cl)nc2c1] | white solid, yield: 26.7% | ¹H NMR (400 MHz, DMSO-d₆) δ 13.34 (s, 1H), 9.00 (s, 1H), 7.95-7.86 (m, 1H), 7.76 (dd, J = 8.0, 1.5 Hz, 1H), 7.64-7.56 (m, 1H), 7.47-7.18 (m, 3H), 4.66-4.53 (m, 1H), 3.49-3.37 (m, 2H), 1.26 (s, 3H), 1.23 (s, 3H). | 402 |
| Example 107 Method 16 | 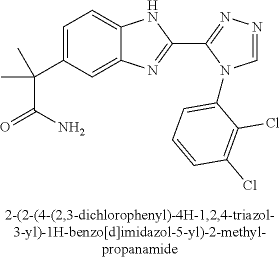 [SMILES: CC(C)(C(N)=O)c1ccc2[nH]c(-c3nncn3-c3cccc(Cl)c3Cl)nc2c1] | white solid, yield: 17.4% | ¹H NMR (400 MHz, DMSO-d₆) δ 13.47 (s, 1H), 9.01 (s, 1H), 7.91 (d, J = 8.0 Hz, 1H), 7.77 (dd, J = 8.0, 1.6 Hz, 1H), 7.60 (t, J = 8.0 Hz, 1H), 7.52-7.33 (m, 2H), 7.30-7.09 (m, 1H), 6.80 (d, J = 26.8 Hz, 2H), 1.45 (d, J = 8.0 Hz, 6H). | 415 |
| Example 110 Method 16 | 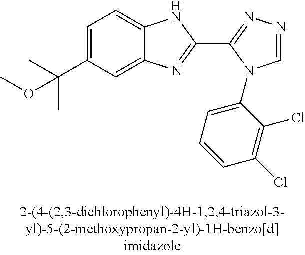 [SMILES: COC(C)(C)c1ccc2[nH]c(-c3nncn3-c3cccc(Cl)c3Cl)nc2c1] | white solid, yield: 82% | ¹H NMR (400 MHz, Chloroform-d) δ 13.35 (br s, 1H), 8.41 (s, 1H), 8.19-7.30 (m, 6H), 3.05 (s, 3H), 1.59 (s, 6H). | 402 |
| Example 111 Method 16 | 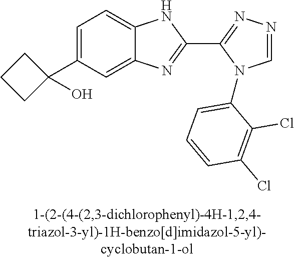 [SMILES: OC1(c2ccc3[nH]c(-c4nncn4-c4cccc(Cl)c4Cl)nc3c2)CCC1] | white solid, yield: 24% | ¹H NMR (400 MHz, DMSO-d₆) δ 13.47 (s, 1H), 9.02 (s, 1H), 7.94-7.89 (m, 1H), 7.78 (dd, J = 8.0, 1.6 Hz, 1H), 7.67-7.27 (m, 4H), 5.43 (s, 1H), 2.39-2.16 (m, 3H), 2.08-1.52 (m, 3H). | 400 |
| Example 112 Method 16 | 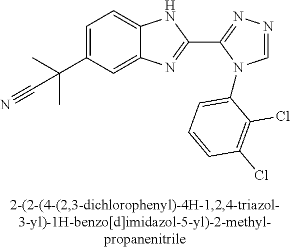 [SMILES: CC(C)(C#N)c1ccc2[nH]c(-c3nncn3-c3cccc(Cl)c3Cl)nc2c1] | white solid, yield: 25.6% | ¹H NMR (400 MHz, DMSO-d₆) δ 13.66 (s, 1H), 9.04 (d, J = 1.2 Hz, 1H), 7.92 (ddd, J = 8.4, 4.8, 1.6 Hz, 1H), 7.78 (dt, J = 8.0, 1.2 Hz, 1H), 7.65-7.55 (m, 2H), 7.54-7.44 (m, 2H), 7.32 (dd, J = 8.8, 2.0 Hz, 1H), 5.32 (t, J = 4.8 Hz, 1H), 1.71 (d, J = 12.4 Hz, 6H). | 396 |
| Example 113 Method 16 | 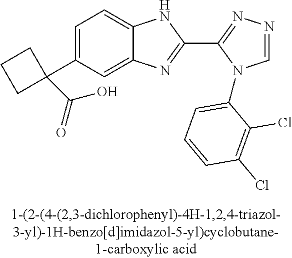 [SMILES: O=C(O)C1(c2ccc3[nH]c(-c4nncn4-c4cccc(Cl)c4Cl)nc3c2)CCC1] | white solid, yield: 15% | ¹H NMR (400 MHz, Methanol-d₄) δ 8.78 (s, 1H), 7.77-7.71 (m, 1H), 7.56 (dd, J = 8.0, 1.6 Hz, 1H), 7.51-7.40 (m, 3H), 7.23-7.12 (m, 1H), 2.90-2.73 (m, 2H), 2.56-2.40 (m, 2H), 2.09-1.93 (m, 1H), 1.90-1.77 (m, 1H). | 428 |
| Example 129 Method 16 | 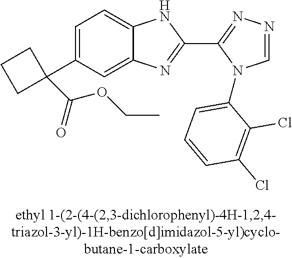 [SMILES: CCOC(=O)C1(c2ccc3[nH]c(-c4nncn4-c4cccc(Cl)c4Cl)nc3c2)CCC1] | white solid, yield: 68% | ¹H NMR (400 MHz, DMSO-d₆) δ 13.54 (s, 1H), 9.03 (s, 1H), 7.92 (dd, J = 8.2, 1.5 Hz, 1H), 7.77 (dd, J = 8.0, 1.5 Hz, 1H), 7.60 (t, J = 8.1 Hz, 1H), 7.49- 7.40 (m, 1H), 7.36- 7.27 (m, 1H), 7.15- 7.02 (m, 1H), 4.01 (q, J = 7.1 Hz, 2H), 2.79- 2.69 (m, 2H), 2.47- 2.39 (m, 2H), 2.01- 1.76 (m, 2H), 1.09- 1.04 (m, 3H). | 456 |
| Example 130 Method 16 | 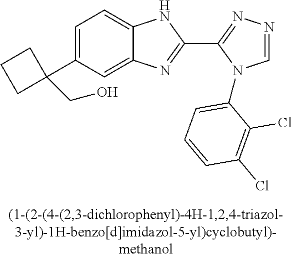 [SMILES: OCC1(c2ccc3[nH]c(-c4nncn4-c4cccc(Cl)c4Cl)nc3c2)CCC1] | white solid, yield: 18% | ¹H NMR (400 MHz, DMSO-d₆) δ 13.42- 13.35 (m, 1H), 9.01 (s, 1H), 7.95-7.88 (m, 1H), 7.82-7.73 (m, 1H), 7.63-7.55 (m, 1H), 7.45-7.32 (m, 1H), 7.23-7.08 (m, 1H), 7.05-6.87 (m, 1H), 4.78-4.50 (m, 1H), 3.55-3.45 (m, 2H), 2.32-2.13 (m, 4H), 2.08-1.70 (m, 3H). | 414 |
| Example 133 Method 16 | 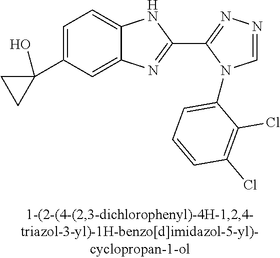 [SMILES: OC1(c2ccc3[nH]c(-c4nncn4-c4cccc(Cl)c4Cl)nc3c2)CC1] | white solid, yield: 8.3% | ¹H NMR (400 MHz, DMSO-d6) δ 13.41 (s, 1H), 9.00 (s, 1H), 7.91 (dd, J = 8.1, 1.5 Hz, 1H), 7.76 (dd, J = 7.9, 1.5 Hz, 1H), 7.59 (t, J = 8.1 Hz, 1H), 7.56- 7.28 (m, 2H), 7.18- 6.83 (m, 1H), 6.03- 5.77 (m, 1H), 1.15- 1.02 (m, 2H), 0.97- 0.88 (m, 2H). | 386 |
| Example 134 Method 16 | 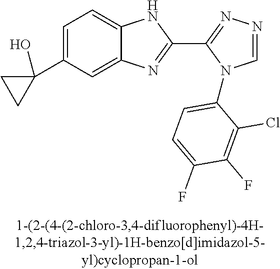 [SMILES: OC1(c2ccc3[nH]c(-c4nncn4-c4ccc(F)c(F)c4Cl)nc3c2)CC1] | white solid, yield: 33% | ¹H NMR (400 MHz, DMSO-d₆) δ 8.99 (s, 1H), 7.83-7.73 (m, 2H), 7.52-7.44 (m, 1H), 7.38 (d, J = 8.5 Hz, 1H), 7.05-6.95 (m, 1H), 1.13-1.03 (m, 2H), 0.97-0.89 (m, 2H). | 388 |
| Example 135 Method 16 | 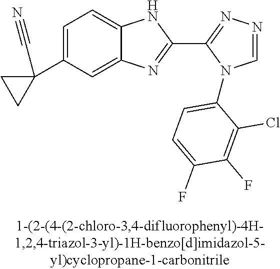 [SMILES: N#CC1(c2ccc3[nH]c(-c4nncn4-c4ccc(F)c(F)c4Cl)nc3c2)CC1] | white solid, yield: 28% | ¹H NMR (400 MHz, DMSO-d₆) δ 13.67 (s, 1H), 9.01 (s, 1H), 7.79-7.73 (m, 2H), 7.55-7.07 (m, 3H), 1.80-1.61 (m, 2H), 1.54-1.48 (m, 2H). | 397 |

### Preparation of 1-(2-(4-(2,3-dichlorophenyl)-4H-1,2,4-triazol-3-yl)-1H-benzo[d]imidazol-5-yl)pyrrolidin-2-one (137) Method 17

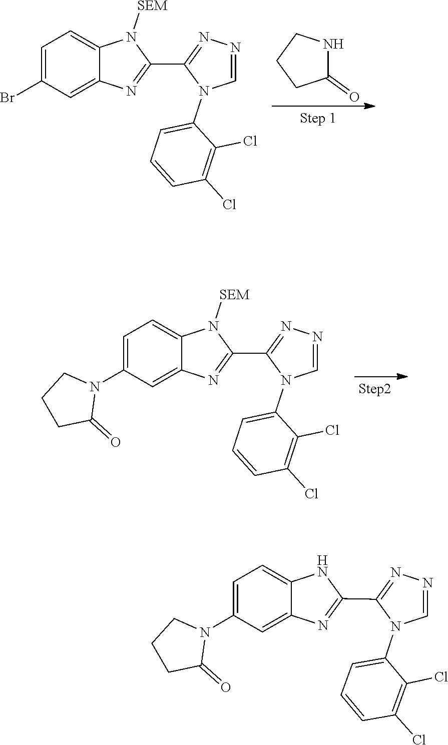

### Step 1. Preparation of 1-(2-(4-(2,3-dichlorophenyl)-4H-1,2,4-triazol-3-yl)-1-((2-(trimethylsilyl)ethoxy)methyl)-1H-benzo[d]imidazol-5-yl)pyrrolidin-2-one (3)

A mixture solution of 5-bromo-2-(4-(2,3-dichlorophenyl)-4H-1,2,4-triazol-3-yl)-1-((2-(trimethylsilyl)ethoxy)methyl)-1H-benzo[d]imidazole (100 mg, 0.1859 mmol), pyrrolidin-2-one (18.9 mg, 0.2230 mmol), Cs₂CO₃ (12.06 mg, 0.3718 mmol), Pd₂(dba)₃ (1.69 mg, 0.0186 mmol), X-phos (1.76 mg, 0.0372 mmol) and 1,4-Dio (10 mL) was stirred at 100° C. for 16 hrs under N₂ atmosphere. After the reaction was completed, the mixture was diluted with water (10 mL), and extracted three times with EA (10 mL). The organic layers were combined, dried over anhydrous Na₂SO₄ and concentrated under reduced pressure to give a crude product that was purified through column chromatography on silica gel (MeOH:DCM=1:10) to give the 1-(2-(4-(2,3-dichlorophenyl)-4H-1,2,4-triazol-3-yl)-1-((2-(trimethylsilyl)ethoxy)methyl)-1H-benzo[d]imidazol-5-yl)pyrrolidin-2-one (74 mg, 73.27% yield) as a oil. MS (ESI) m/z 543.1 [M+H]⁺.

### Step 2. Preparation of 1-(2-(4-(2,3-dichlorophenyl)-4H-1,2,4-triazol-3-yl)-1H-benzo[d]imidazol-5-yl)pyrrolidin-2-one

1-(2-(4-(2,3-dichlorophenyl)-4H-1,2,4-triazol-3-yl)-1-((2-(trimethylsilyl)ethoxy)methyl)-1H-benzo[d]imidazol-5-yl)pyrrolidin-2-one (74 mg, 0.1363 mmol) and TFA/DCM (3/3 mL) was stirred at 25° C. for 2 hrs. After the reaction was completed, the mixture was concentrated under reduced pressure to give a crude product that was purified by perp-HPLC (column-Gemini-C18 150×21.2 mm, 5 um; Mobile phase: ACN-H₂O (0.1% TFA), 30%-50%) to afford the desired product SIR-00015146 (1.4 mg, 2.49% yield) as a white solid. MS (ESI) m/z 413.1 [M+H]⁺.

¹H NMR (400 MHz) δ 8.82 (s, 1H), 7.78 (dd, J=8.0, 1.4 Hz, 1H), 7.73 (s, 1H), 7.59 (dd, J=8.0, 1.2 Hz, 1H), 7.51 (d, J=8.2 Hz, 2H), 7.48-7.41 (m, 1H), 3.59-3.42 (m, 2H), 2.58 (t, J=8.0 Hz, 2H), 2.22-2.14 (m, 2H).

| Example | | | | |
|---|---|---|---|---|
| No. (Compound | | Appearance | | MS |
| No.) | | and | | (m/z) |
| Method | Structure and Name | Yield | ¹H NMR Data | [M + H]⁺ |
| Example 136 Method 9 | 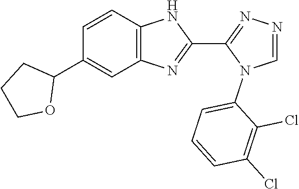 [SMILES: Clc1cccc(-n2cnnc2-c2nc3cc(C4CCCO4)ccc3[nH]2)c1Cl] | white solid, yield: 11% | ¹H NMR (400 MHz, DMSO-d₆) δ 13.52 (s, 1H), 9.03 (s, 1H), 7.92 (dd, J = 8.2, 1.2 Hz, 1H), 7.78 (d, J = 7.0 Hz, 1H), 7.60 (t, J = 8.0 Hz, 1H), 7.39 (dd, J = 24.0, 16.6 Hz, 2H), 7.17 (d, J = 58.8 Hz, 1H), 4.86 (d, J = 24.8 Hz, 1H), 3.90 (d, J = 74.0 Hz, 2H), 2.29 (s, 1H), 1.94 (s, 2H), 1.64 (dq, J = 12.0, 8.0 Hz, 1H). | 400 |
| | 2-(4-(2,3- | | | |
| | dichlorophenyl)-4H- | | | |
| | 1,2,4-triazol-3-yl)-5- | | | |
| | (tetrahydrofuran-2-yl)- | | | |
| | 1H-benzo[d]imidazole | | | |
| Example 137 Method 17 | 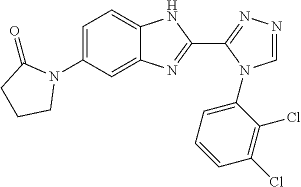 [SMILES: O=C1CCCN1c1ccc2[nH]c(-c3nncn3-c3cccc(Cl)c3Cl)nc2c1] | white solid, yield: 3% | ¹H NMR (400 MHz MeOD-d₄) δ 8.82 (s, 1H), 7.78 (dd, J = 8.0, 1.4 Hz, 1H), 7.73 (s, 1H), 7.59 (dd, J = 8.0, 1.2 Hz, 1H), 7.51 (d, J = 8.2 Hz, 2H), 7.48-7.41 (m, 1H), 3.59- 3.42 (m, 2H), 2.58 (t, J = 8.0 Hz, 2H), 2.22-2.14 (m, 2H). | 413 |
| | 1-(2-(4-(2,3- | | | |
| | dichlorophenyl)-4H- | | | |
| | 1,2,4-triazol-3-yl)-1H- | | | |
| | benzo[d]imidazol-5- | | | |
| | yl)pyrrolidin-2-one | | | |
| Example 138 Method 14 | 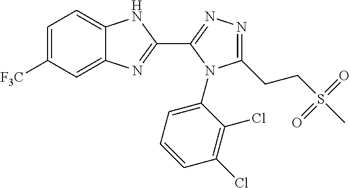 [SMILES: CS(=O)(=O)CCc1nnc(-c2nc3cc(C(F)(F)F)ccc3[nH]2)n1-c1cccc(Cl)c1Cl] | white solid, yield: 50% | ¹H NMR (400 MHz, DMSO-d₆) δ 14.07 (bs, 1H), 7.97 (dd, J = 1.0 Hz, 8.0 Hz, 1H), 7.85 (dd, J = 1.0 Hz, 8.0 Hz, 1H), 7.79 (s, 1H), 7.71- 7.64 (m, 2H), 7.53 (d, J = 8.4 Hz, 1H), 3.74- 3.61 (m, 2H), 3.06 (s, 3H), 3.04-2.88 (m, 2H). | 504 |
| | 2-(4-(2,3- | | | |
| | dichlorophenyl)-5-(2- | | | |
| | (methylsulfonyl)ethyl)- | | | |
| | 4H-1,2,4-triazol-3-yl)- | | | |
| | 5-(trifluoromethyl)-1H- | | | |
| | benzo[d]imidazole | | | |
| Example 139 Method 14 | 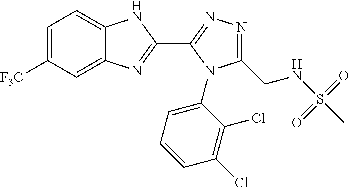 [SMILES: CS(=O)(=O)NCc1nnc(-c2nc3cc(C(F)(F)F)ccc3[nH]2)n1-c1cccc(Cl)c1Cl] | white solid, yield: 16% | ¹H NMR (400 MHz, DMSO-d₆) δ 13.97 (s, 1H), 7.91 (dd, J = 8.2, 1.4 Hz, 1H), 7.80- 7.75 (m, 2H), 7.70-7.58 (m, 3H), 7.49 (d, J = 8.2 Hz, 1H), 4.54-3.97 (m, 2H), 2.88 (s, 3H). | 505 |
| | N-((4-(2,3- | | | |
| | dichlorophenyl)-5-(5- | | | |
| | (trifluoromethyl)-1H- | | | |
| | benzo[d]imidazol-2-yl)- | | | |
| | 4H-1,2,4-triazol-3- | | | |
| | yl)methyl)methanesulfo | | | |
| | namide | | | |
| Example 140 Method 9 | 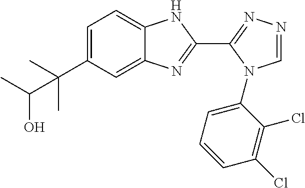 [SMILES: CC(O)C(C)(C)c1ccc2[nH]c(-c3nncn3-c3cccc(Cl)c3Cl)nc2c1] | yellow solid, yield: 74.9% | ¹H NMR (400 MHz, Methanol-d₄) δ 8.73 (s, 1H), 7.71 (dd, J = 8.4, 1.6 Hz, 1H), 7.56-7.19 (m, 5H), 3.81 (s, 1H), 1.27 (d, J = 9.2 Hz, 6H), 0.81 (d, J = 7.2 Hz, 3H). | 416 |
| | 3-(2-(4-(2,3- | | | |
| | dichlorophenyl)-4H- | | | |
| | 1,2,4-triazol-3-yl)-1H- | | | |
| | benzo[d]imidazol-5-yl)- | | | |
| | 3-methylbutan-2-ol | | | |
| Example 141 Method 9 | 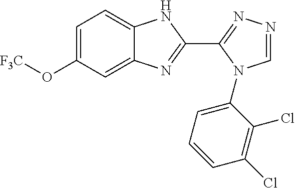 [SMILES: FC(F)(F)Oc1ccc2[nH]c(-c3nncn3-c3cccc(Cl)c3Cl)nc2c1] | white solid, yield: 6% | ¹H NMR (400 MHz, DMSO-d₆) δ 13.86 (s, 1H), 9.06 (s, 1H), 7.92 (dd, J = 8.2, 1.5 Hz, 1H), 7.79 (dd, J = 8.0, 1.5 Hz, 1H), 7.60 (t, J = 8.1 Hz, 2H), 7.46 (s, 1H), 7.21 (d, J = 57.7 Hz, 1H). | 414 |
| | Preparation of 2-(4- | | | |
| | (2,3-dichlorophenyl)- | | | |
| | 4H-1,2,4-triazol-3-yl)- | | | |
| | 5-(trifluoromethoxy)- | | | |
| | 1H-benzo[d]imidazole | | | |
| Example 142 Method 1 | 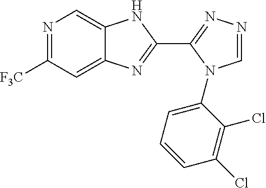 [SMILES: FC(F)(F)c1cc2nc(-c3nncn3-c3cccc(Cl)c3Cl)[nH]c2cn1] | white solid, yield: 2% | ¹H NMR (400 MHz, DMSO-d₆) δ 9.09 (s, 1H), 8.90 (s, 1H), 7.92-7.89 (m, 2H), 7.77 (dd, J = 8.0, 1.2 Hz, 1H), 7.57 (t, J = 8.0 Hz, 1H). | 399 |
| | 2-(4-(2,3- | | | |
| | dichlorophenyl)-4H- | | | |
| | 1,2,4-triazol-3-yl)-6- | | | |
| | (trifluoromethyl)-3H- | | | |
| | imidazo[4,5-c]pyridine | | | |
| Example 143 Method 14 | 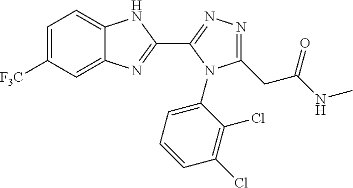 [SMILES: CNC(=O)Cc1nnc(-c2nc3cc(C(F)(F)F)ccc3[nH]2)n1-c1cccc(Cl)c1Cl] | white solid, | ¹H NMR (400 MHz, DMSO-d₆) δ 14.04 (b, 1H), 7.98-7.88 (m, 2H), 7.78 (s, 1H), 7.70- 7.46 (m, 4H), 3.77 (d, J = 16.1 Hz, 1H), 3.46 (d, J = 16.1 Hz, 1H), 2.47 (d, J = 4.6 Hz, 3H). | 469 |
| | 2-(4-(2,3- | | | |
| | Dichlorophenyl)-5-(5- | | | |
| | (trifluoromethyl)-1H- | | | |
| | benzo[d]imidazol-2-yl)- | | | |
| | 4H-1,2,4-triazol-3-yl)- | | | |
| | N-methylacetamide | | | |
| Example 144 Method 14 | 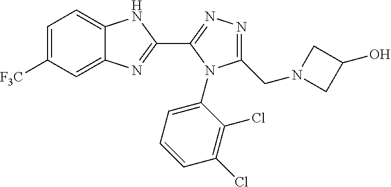 [SMILES: OC1CN(Cc2nnc(-c3nc4cc(C(F)(F)F)ccc4[nH]3)n2-c2cccc(Cl)c2Cl)C1] | white solid, yield: 16% | ¹HNMR (400 MHz, DMSO-d₆) δ 14.10 (d, J= 11.6 Hz, 1H), 8.02-7.94 (m, 1H), 7.86-7.77 (m 2H), 7.76-7.57 (m, 3H), 5.95 (s, 1H), 4.60-4.10 (m, 5H), 3.90-3.60 (m, 2H). | 483 |
| | 1-((4-(2,3- | | | |
| | dichlorophenyl)-5-(5- | | | |
| | (trifluoromethyl)-1H- | | | |
| | benzo[d]imidazol-2-yl)- | | | |
| | 4H-1,2,4-triazol-3- | | | |
| | yl)methyl)azetidin-3-ol | | | |
| Example 145 Method 14 | 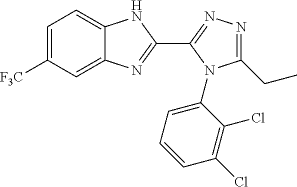 [SMILES: CCc1nnc(-c2nc3cc(C(F)(F)F)ccc3[nH]2)n1-c1cccc(Cl)c1Cl] | white solid, yield: 76% | H NMR (400 MHz, DMSO-d₆) δ 13.99 (d, J = 8.8 Hz, 1H), 7.95 (dd, J = 8.2 Hz, 1.4 Hz, 1H), 7.84-7.74 (m, 2H), 7.85- 7.40 (m, 3H), 2.64- 2.53 (m, 2H), 1.22 (t, J = 7.6 Hz, 3H). | 426 |
| | 2-(4-(2,3- | | | |
| | dichlorophenyl)-5- | | | |
| | ethyl-4H-1,2,4-triazol- | | | |
| | 3-yl)-5- | | | |
| | (trifluoromethyl)-1H- | | | |
| | benzo[d]imidazole | | | |
| Example 146 Method 14 single unknown stereoisomer | 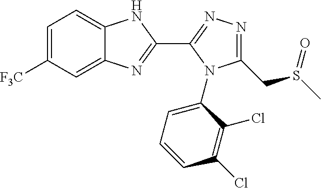 | white solid, | ¹H NMR (400 MHz, DMSO-d₆) δ 14.00 (s, 1H), 7.94 (dd, J = 1.4 Hz, 8.0 Hz, 1H), 7.96-7.75 (m, 2H), 7.70-7.60 (m, 2H), 7.56-7.47 (m, 1H), 4.38 (d, J = 14.4 Hz, 1H), 4.06 (d, J = 14.4 Hz, 1H), 2.70 (s, 3H). | 474 Xbridge @Prep C18 5 μM OBDTM 19 × 150 mm 45% ACN in H₂O (0.1 % FA) RT = 9.93 min |
| | 2-(4-(2,3- | | | |
| | dichlorophenyl)-5- | | | |
| | ((methylsulfinyl)methyl)- | | | |
| | 4H-1,2,4-triazol-3-yl)- | | | |
| | 5-(trifluoromethyl)-1H- | | | |
| | benzo[d]imidazole | | | |
| Example 147 Method 14 single unknown stereoisomer | 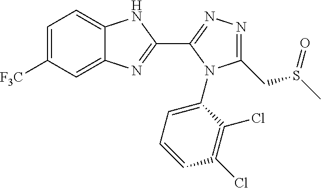 | white solid, | ¹H NMR (400 MHz, DMSO-d₆) δ 14.06 (s, 1H), 7.94 (dd, J = 1.4 Hz, 8.0 Hz, 1H), 7.82-7.74 (m, 2H), 7.72-7.47 (m, 3H), 4.29 (d, J = 14.4 Hz, 1H), 4.18 (d, J = 14.4 Hz, 1H), 2.73 (s, 3H). | 474 Xbridge @Prep C18 5 μM OBDTM 19 ×150 mm 45% ACN in H₂O (0.1 % FA) RT = 10.60 min |
| | 2-(4-(2,3- | | | |
| | dichlorophenyl)-5- | | | |
| | ((methylsulfinyl)methyl)- | | | |
| | 4H-1,2,4-triazol-3-yl)- | | | |
| | 5-(trifluoromethyl)-1H- | | | |
| | benzo[d]imidazole | | | |
| Example 148 Method 1 | 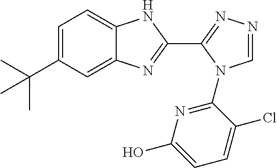 [SMILES: CC(C)(C)c1ccc2[nH]c(-c3nncn3-c3nc(O)ccc3Cl)nc2c1] | white solid, yield: 17% | ¹H NMR (400 MHz, MeOD) δ 8.89 (s, 1H), 7.84 (d, J = 8.8 Hz, 1H), 7.57 (d, J = 1.7 Hz, 1H), 7.48 (d, J = 8.7 Hz, 1H), 7.40 (dd, J = 8.6, 1.8 Hz, 1H), 6.90 (d, J = 8.8 Hz, 1H), 1.37 (s, 9H). | 369 |
| | 6-(3-(5-(tert-butyl)-1H- | | | |
| | benzo[d]imidazol-2-yl)- | | | |
| | 4H-1,2,4-triazol-4-yl)- | | | |
| | 5-chloropyridin-2(1H)- | | | |
| | one | | | |
| Example 149 Method 1 | 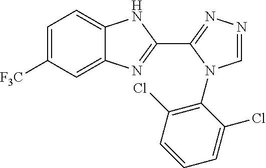 [SMILES: FC(F)(F)c1ccc2[nH]c(-c3nncn3-c3c(Cl)cccc3Cl)nc2c1] | white solid, yield: 19% | ¹HNMR (400 MHz, DMSO-d₆) δ 14.13 (d, J = 9.2 Hz, 1H), 9.16 (s, 1H), 7.85-7.74 (m, 3H), 7.74- 7.54 (m, 3H) | 398 |
| | 2-(4-(2,6- | | | |
| | dichlorophenyl)-4H- | | | |
| | 1,2,4-triazol-3-yl)-5- | | | |
| | (trifluoromethyl)-1H- | | | |
| | benzo[d]imidazole | | | |
| Example 150 Method 1 | 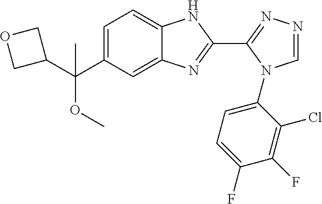 [SMILES: COC(C)(c1ccc2[nH]c(-c3nncn3-c3ccc(F)c(F)c3Cl)nc2c1)C1COC1] | yellow solid, yield: 58.1% | ¹H NMR (400 MHz, Methanol-d₄) δ 8.84 (s, 1H), 7.63-7.44 (m, 4H), 7.29 (m, 1H), 4.86 (m, 1H), 4.75-4.55 (m, 2H), 4.45 (m, 1H), 3.37 (m, 1H), 3.17 (d, J = 18.3 Hz, 3H), 1.65 (d, J = 10.3 Hz, 3H). | 446 |
| | 2-(4-(2-chloro-3,4- | | | |
| | difluorophenyl)-4H- | | | |
| | 1,2,4-triazol-3-yl)-5-(1- | | | |
| | methoxy-1-(oxetan-3- | | | |
| | yl)ethyl)-1H- | | | |
| | benzo[d]imidazole | | | |
| Example 151 Method 9 | 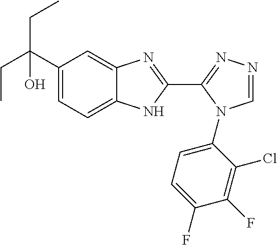 [SMILES: CCC(O)(CC)c1ccc2[nH]c(-c3nncn3-c3ccc(F)c(F)c3Cl)nc2c1] | yellow solid, yield: 6% | ¹H NMR (400 MHz, Methanol-d₄) δ 8.73 (s, 1H), 7.60-7.47 (m, 1H), 7.46-7.34 (m, 3H), 7.31-7.11 (m, 1H), 1.83-1.70 (m, 4H), 0.69-0.57 (m, 6H). | 418 |
| | 3-(2-(4-(2-chloro-3,4- | | | |
| | difluorophenyl)-4H- | | | |
| | 1,2,4-triazol-3-yl)-1H- | | | |
| | benzo[d]imidazol-5- | | | |
| | yl)pentan-3-ol | | | |
| Example 152 Method 9 | 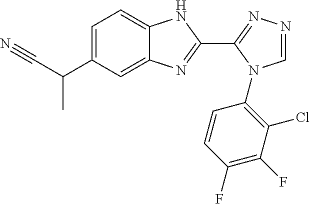 [SMILES: CC(C#N)c1ccc2[nH]c(-c3nncn3-c3ccc(F)c(F)c3Cl)nc2c1] | yellow solid, yield: 20.8% | ¹H NMR (400 MHz, DMSO-d₆) δ 13.69 (s, 1H), 9.02 (s, 1H), 7.86- 7.63 (m, 2H), 7.65-7.46 (m, 2H), 7.26 (d, J = 46.4 Hz, 1H), 4.37 (d, J = 38.8 Hz, 1H), 1.56 (d, J = 7.2 Hz, 3H). | 385 |
| | 2-(2-(4-(2-chloro-3,4- | | | |
| | difluorophenyl)-4H- | | | |
| | 1,2,4-triazol-3-yl)-1H- | | | |
| | benzo[d]imidazol-5- | | | |
| | yl)propanenitrile | | | |
| Example 153 Method 9 | 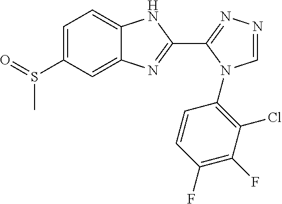 [SMILES: CS(=O)c1ccc2[nH]c(-c3nncn3-c3ccc(F)c(F)c3Cl)nc2c1] | white solid, yield: 22.3% | ¹H NMR (400 MHz, DMSO-d₆) δ 9.06 (s, 1H), 7.92 (dd, J = 8.4, 1.6 Hz, 1H), 7.79 (dd, J = 8.0, 1.6 Hz, 2H), 7.72- 7.56 (m, 2H), 7.49 (m, 1H), 2.72 (s, 3H). | 392 |
| | 2-(4-(2,3- | | | |
| | dichlorophenyl)-4H- | | | |
| | 1,2,4-triazol-3-yl)-5- | | | |
| | (methylsulfinyl)-1H- | | | |
| | benzo[d]imidazole | | | |
| Example 154 Method 9 | 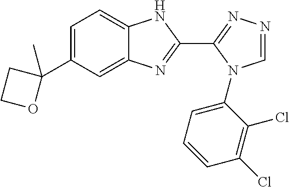 [SMILES: CC1(c2ccc3[nH]c(-c4nncn4-c4cccc(Cl)c4Cl)nc3c2)CCO1] | yellow solid, yield: 5% | ¹H NMR (400 MHz, DMSO-d₆) δ 13.49 (s, 1H), 9.03 (s, 1H), 7.92 (d, J = 7.8 Hz, 1H), 7.77 (d, J = 8.0 Hz, 1H), 7.60 (t, J = 8.0 Hz, 1H), 7.39 (s, 1H), 7.32 (s, 1H), 7.10 (s, 1H), 4.69 (d, J = 7.2 Hz, 1H), 4.42 (s, 1H), 2.33 (s, 1H), 2.00 (d, J = 7.0 Hz, 1H), 1.23 (s, 3H). | 400 |
| | 2-(4-(2,3- | | | |
| | dichlorophenyl)-4H- | | | |
| | 1,2,4-triazol-3-yl)-5-(2- | | | |
| | methyloxetan-2-yl)-1H- | | | |
| | benzo[d]imidazole | | | |
| Example 155 Method 9 | 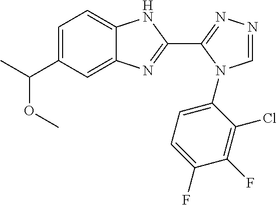 [SMILES: COC(C)c1ccc2[nH]c(-c3nncn3-c3ccc(F)c(F)c3Cl)nc2c1] | yellow solid, yield: 66.2% | ¹H NMR (400 MHz, Methanol-d₄) δ 8.83 (s, 1H), 7.63-7.43 (m, 4H), 7.25 (m, 1H), 4.43 (m, 1H), 3.20 (s, 3H), 1.43 (d, J = 6.4 Hz, 3H). | 390 |
| | 2-(4-(2-chloro-3,4- | | | |
| | difluorophenyl)-4H- | | | |
| | 1,2,4-triazol-3-yl)-5-(1- | | | |
| | methoxyethyl)-1H- | | | |
| | benzo[d]imidazole | | | |
| Example 156 Method 14 | 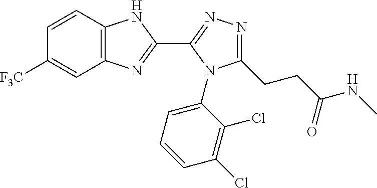 [SMILES: CNC(=O)CCc1nnc(-c2nc3cc(C(F)(F)F)ccc3[nH]2)n1-c1cccc(Cl)c1Cl] | yellow oil, yield: 15% | ¹HNMR (400 MHz, DMSO-d₆) δ 13.95 (s, 1H), 7.95 (dd, J = 8.2 Hz, 1.4 Hz, 1H), 7.90- 7.83 (m, 1H), 7.81-7.76 (m, 2H), 7.71-7.61 (m, 2H), 7.55 (bs, 1H), 2.82- 2.57 (m, 5H), 2.57- 2.53 (m, 2H). | 484 |
| | 3-(4-(2,3- | | | |
| | dichlorophenyl)-5-(5- | | | |
| | (trifluoromethyl)-1H- | | | |
| | benzo[d]imidazol-2-yl)- | | | |
| | 4H-1,2,4-triazol-3-yl)- | | | |
| | N-methylpropanamide | | | |
| Example 157 Method 14 | 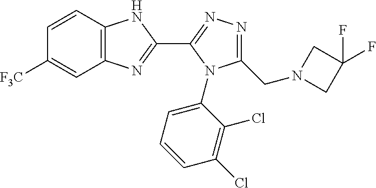 [SMILES: FC1(F)CN(Cc2nnc(-c3nc4cc(C(F)(F)F)ccc4[nH]3)n2-c2cccc(Cl)c2Cl)C1] | white solid, | ¹H NMR (400 MHz, DMSO-d₆) δ 14.00 (s, 1H), 7.92 (dd, J = 1.2 Hz, 8.0 Hz, 1H), 7.82- 7.76 (m, 2H), 7.73-7.48 (m, 3H), 3.88-3.76 (m, 2H), 3.65 (t, J = 12.4 Hz, 4H) | 503 |
| | 2-(4-(2,3- | | | |
| | dichlorophenyl)-5- | | | |
| | ((3,3-difluoroazetidin- | | | |
| | 1-yl)methyl)-4H-1,2,4- | | | |
| | triazol-3-yl)-5- | | | |
| | (trifluoromethyl)-1H- | | | |
| | benzo[d]imidazole | | | |
| Example 158 Method 9 | 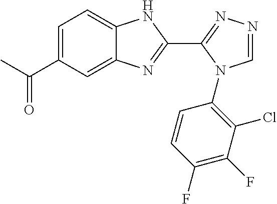 [SMILES: CC(=O)c1ccc2[nH]c(-c3nncn3-c3ccc(F)c(F)c3Cl)nc2c1] | white solid, yield: 36% | ¹H NMR (400 MHz, DMSO-d₆) δ 13.96 (s, 1H), 9.05 (s, 1H), 8.14 (s, 1H), 8.02-7.73 (m, 3H), 7.59 (d, J = 8.5 Hz, 1H), 2.61 (s, 3H). | 374 |
| | 1-(2-(4-(2-chloro-3,4- | | | |
| | difluorophenyl)-4H- | | | |
| | 1,2,4-triazol-3-yl)-1H- | | | |
| | benzo[d]imidazol-5- | | | |
| | yl)ethan-1-one | | | |
| Example 159 Method 1 | 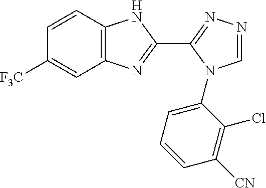 [SMILES: N#Cc1cccc(-n2cnnc2-c2nc3cc(C(F)(F)F)ccc3[nH]2)c1Cl] | white solid, yield: 16% | ¹H NMR (400 MHz, DMSO-d₆) δ 14.08 (bs, 1H), 9.10 (s, 1H), 8.27 (dd, J = 8.0 Hz, J = 1.2 Hz, 1H), 8.17 (d, J = 8.0 Hz, J = 1.2 Hz, 1H), 7.84- 7.76 (m, 2H), 7.75- 7.42 (m, 2H). | 389 |
| | 2-chloro-3-(3-(5- | | | |
| | (trifluoromethyl)-1H- | | | |
| | benzo[d]imidazol-2-yl)- | | | |
| | 4H-1,2,4-triazol-4- | | | |
| | yl)benzonitrile | | | |
| Example 160 Method 1 | 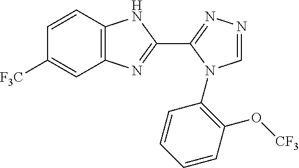 [SMILES: FC(F)(F)Oc1ccccc1-n1cnnc1-c1nc2cc(C(F)(F)F)ccc2[nH]1] | yellow solid, yield: 7% | ¹H NMR (400 MHz, DMSO-d₆) δ 14.02 (bs, 1H), 9.10 (s, 1H), 7.87 (dd, J = 8.0 Hz, J = 2.0 Hz, 1H), 7.81 (s, 1H), 7.78-7.72 (m, 1H), 7.72- 7.42 (m, 3H). | 414 |
| | (trifluoromethoxy) | | | |
| | phenyl)-4H-1,2,4-triazol-3- | | | |
| | yl)-5-(trifluoromethyl)- | | | |
| | 1H-benzo[d]imidazole | | | |
| Example 161 Method 14 single unknown stereoisomer | 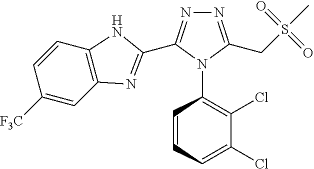 | yellow solid, | ¹H NMR (400 MHz, DMSO-d₆) δ 13.88 (bs, 1H), 12.95 (bs, 1H), 8.90 (s, 1H), 7.98 (d, J = 5.6 Hz, 1H), 7.80-7.44 (m, 6H), 5.85 (s, 1H). | 491 Note: CHIRAL PAK OJ-H 250 mm ×20mm, 5 μm. 40% EtOH (NH4OH 0.2%):60 % CO2 RT = 2.06 min |
| | 2-(4-(2-chloro-6-(1H- | | | |
| | pyrazol-5-yl)phenyl)- | | | |
| | 4H-1,2,4-triazol-3-yl)- | | | |
| | 5-(trifluoromethyl)-1H- | | | |
| | benzo[d]imidazole | | | |
| Example 162 Method 14 single unknown stereoisomer | 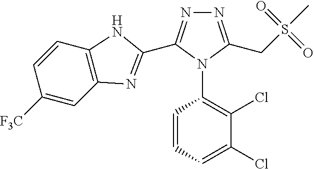 | yellow solid, | ¹H NMR (400 MHz, DMSO-d₆) δ 13.88 (bs, 1H), 12.95 (bs, 1H), 8.90 (s, 1H), 7.98 (d, J = 5.6 Hz, 1H), 7.80-7.44 (m, 6H), 5.85 (s, 1H). | 491 Note: CHIRAL PAK OJ-H 250 mm × 20 mm, 5 μm. 40% EtOH (NH4OH 0.2%):60 % CO2 RT = 2.34 min |
| | 2-(4-(2-chloro-6-(1H- | | | |
| | pyrazol-5-yl)phenyl)- | | | |
| | 4H-1,2,4-triazol-3-yl)- | | | |
| | 5-(trifluoromethyl)-1H- | | | |
| | benzo[d]imidazole | | | |
| Example 163 Method 1 | 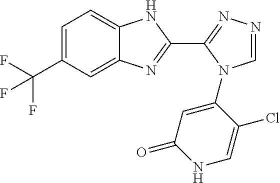 [SMILES: O=c1cc(-n2cnnc2-c2nc3cc(C(F)(F)F)ccc3[nH]2)c(Cl)c[nH]1] | white solid, | ¹H NMR (400 MHz, DMSO-d6) δ 9.04 (s, 1H), 7.96 (d, J = 24.2 Hz, 2H), 7.77 (d, J = 8.5 Hz, 1H), 7.57 (d, J = 8.5 Hz, 1H), 6.96 (s, 1H). | 380 |
| | 5-Chloro-4-(3-(5- | | | |
| | (trifluoromethyl)-1H- | | | |
| | benzo[d]imidazol-2-yl)- | | | |
| | 4H-1,2,4-triazol-4- | | | |
| | yl)pyridin-2(1H)-one | | | |
| Example 164 Method 1 | 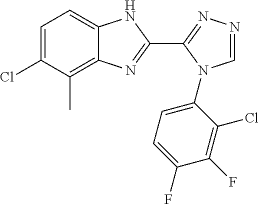 [SMILES: Cc1c(Cl)ccc2[nH]c(-c3nncn3-c3ccc(F)c(F)c3Cl)nc12] | grey solid, yield: 9% | ¹H NMR (400 MHz, DMSO-d₆) δ 13.76 (s, 1H), 9.06 (s, 1H), 7.79- 7.76 (m, 2H), 7.34 (d, J= 8.8 Hz, 2H), 2.24 (s, 3H). | 380 |
| | 5-chloro-2-(4-(2-chloro- | | | |
| | 3,4-difluorophenyl)-4H- | | | |
| | 1,2,4-triazol-3-yl)-4- | | | |
| | methyl-1H- | | | |
| | benzo[d]imidazole | | | |
| Example 165 Method 1 | 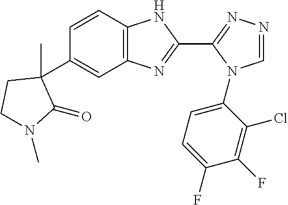 [SMILES: CN1CCC(C)(c2ccc3[nH]c(-c4nncn4-c4ccc(F)c(F)c4Cl)nc3c2)C1=O] | white solid, yield: 9% | ¹H NMR (400 MHz, DMSO-d₆) δ 13.47 (s, 1H), 9.00 (s, 1H), 7.85- 7.66 (m, 2H), 7.44 (d, J = 8.4 Hz, 2H), 7.23 (s, 1H), 3.30-3.17 (m, 2H), 2.80 (s, 3H), 2.40- 2.13 (m, 2H), 1.41 (s, 3H). | 443 |
| | 3-(2-(4-(2-chloro-3,4- | | | |
| | difluorophenyl)-4H- | | | |
| | 1,2,4-triazol-3-yl)-1H- | | | |
| | benzo[d]imidazol-5-yl)- | | | |
| | 1,3-dimethylpyrrolidin- | | | |
| | 2-one | | | |
| Example 166 Method 13 | 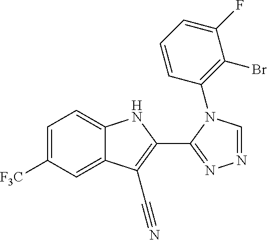 [SMILES: N#Cc1c(-c2nncn2-c2cccc(F)c2Br)[nH]c2ccc(C(F)(F)F)cc12] | white solid, yield: 23% | ¹H NMR (400 MHz, DMSO-d₆) δ 13.40 (s, 1H), 9.24 (s, 1H), 8.79 (s, 1H), 8.53 (s, 1H), 7.96 (s, 1H), 7.80 (d, J = 8.8 Hz, 1H), 7.68 (dd, J = 8.8, 1.2 Hz, 1H), 2.19 (s, 3H). | 403 |
| | 2-(4-(5-chloro-4- | | | |
| | methylpyridin-3-yl)- | | | |
| | 4H-1,2,4-triazol-3-yl)- | | | |
| | 5-(trifluoromethyl)-1H- | | | |
| | indole-3-carbonitrile | | | |
| Example 167 Method 13 | 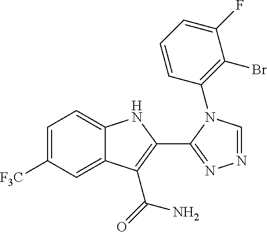 [SMILES: NC(=O)c1c(-c2nncn2-c2cccc(F)c2Br)[nH]c2ccc(C(F)(F)F)cc12] | white solid, yield: 6% | ¹H NMR (400 MHz, DMSO-d₆) δ 12.68 (s, 1H), 9.10 (s, 1H), 8.64 (s, 1H), 8.27 (s, 1H), 8.14 (s, 1H), 7.65 (d, J = 8.0 Hz, 1H), 7.53 (dd, J = 8.4, 1.2 Hz, 1H), 7.13 (s, 1H), 2.13 (s, 3H). | 421 |
| | 2-(4-(5-chloro-4- | | | |
| | methylpyridin-3-yl)- | | | |
| | 4H-1,2,4-triazol-3-yl)- | | | |
| | 5-(trifluoromethyl)-1H- | | | |
| | indole-3-carboxamide | | | |
| Example 168 Method 1 | 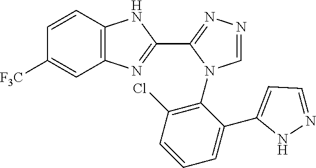 [SMILES: FC(F)(F)c1ccc2[nH]c(-c3nncn3-c3c(Cl)cccc3-c3ccn[nH]3)nc2c1] | white solid, | ¹H NMR (400 MHz, DMSO-d₆) δ 13.88 (bs, 1H), 12.95 (bs, 1H), 8.90 (s, 1H), 7.98 (d, J = 5.6 Hz, 1H), 7.80-7.44 (m, 6H), 5.85 (s, 1H). | 430 |
| | 2-(4-(2-chloro-6-(1H- | | | |
| | pyrazol-5-yl)phenyl)- | | | |
| | 4H-1,2,4-triazol-3-yl)- | | | |
| | 5-(trifluoromethyl)-1H- | | | |
| | benzo[d]imidazole | | | |
| Example 169 Method 13 | 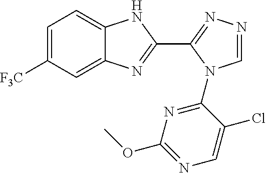 [SMILES: COc1ncc(Cl)c(-n2cnnc2-c2nc3cc(C(F)(F)F)ccc3[nH]2)n1] | white solid, yield: 37.6% | ¹H NMR (400 MHz, Methanol-d₄) δ 8.88 (s, 1H), 8.50 (s, 1H), 8.06 (d, J = 8.4 Hz, 1H), 7.83- 7.68 (m, 2H), 4.05 (s, 3H). | 396 |
| | 2-(4-(5-chloro-2- | | | |
| | methoxypyrimidin-4- | | | |
| | yl)-4H-1,2,4-triazol-3- | | | |
| | yl)-5-(trifluoromethyl)- | | | |
| | 1H-benzo[d]imidazole | | | |
| Example 170 Method 1 | 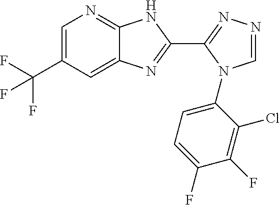 [SMILES: Fc1ccc(-n2cnnc2-c2nc3cc(C(F)(F)F)cnc3[nH]2)c(Cl)c1F] | white solid, yield: 22.3% | ¹H NMR (400 MHz, Methanol-d₄) δ 8.89 (s, 1H), 8.67 (s, 1H), 8.28 (s, 1H), 7.55 (m, 2H). | 401 |
| | 2-(4-(2-chloro-3,4- | | | |
| | difluorophenyl)-4H- | | | |
| | 1,2,4-triazol-3-yl)-6- | | | |
| | (trifluoromethyl)-3H- | | | |
| | imidazo[4,5-b]pyridine | | | |
| Example 171 Method 1 | 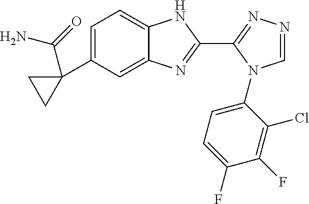 [SMILES: NC(=O)C1(c2ccc3[nH]c(-c4nncn4-c4ccc(F)c(F)c4Cl)nc3c2)CC1] | white solid, | ¹H NMR (400 MHz, DMSO-d₆) δ 13.55 (s, 1H), 9.00 (s, 1H), 7.84- 7.69 (m, 2H), 7.55-7.34 (m, 2H), 7.34-7.06 (m, 1H), 7.04-6.81 (m, 1H), 6.20-5.99 (m, 1H), 1.41-1.28 (m, 2H), 1.03-0.92 (m, 2H). | 415 |
| | 1-(2-(4-(2-chloro-3,4- | | | |
| | difluorophenyl)-4H-1,2,4- | | | |
| | triazol-3-yl)-1H- | | | |
| | benzo[d]imidazol-5- | | | |
| | yl)cyclopropane-1- | | | |
| | carboxamide | | | |
| Example 172 Method 1 | 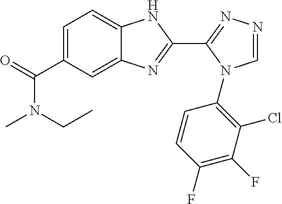 [SMILES: CCN(C)C(=O)c1ccc2[nH]c(-c3nncn3-c3ccc(F)c(F)c3Cl)nc2c1] | white solid, yield: 5.94% | ¹H NMR (400 MHz, DMSO-d₆) δ 13.62 (s, 1H), 9.02 (s, 1H), 7.79- 7.73 (m, 2H), 7.52 (d, J = 8.4 Hz, 1H), 7.47 (s, 1H), 7.21 (d, J = 8.4 Hz, 1H), 2.90 (d, J = 7.6 Hz, 3H), 1.09 (s, 3H). | 417 |
| | 2-(4-(2-chloro-3,4- | | | |
| | difluorophenyl)-4H- | | | |
| | 1,2,4-triazol-3-yl)-N- | | | |
| | ethyl-N-methyl-1H- | | | |
| | benzo[d]imidazole-5- | | | |
| | carboxamide | | | |
| Example 173 Method 1 | 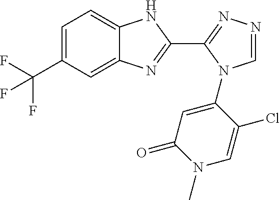 [SMILES: Cn1cc(Cl)c(-n2cnnc2-c2nc3cc(C(F)(F)F)ccc3[nH]2)cc1=O] | white solid, yield: 60 % | ¹H NMR (400 MHz, DMSO-d₆) δ 8.89 (s, 1H), 8.11 (s, 1H), 7.94 (s, 1H), 7.82-7.75 (m, 1H), 7.67- 7.54 (m, 1H), 6.97 (s, 1H), 3.68 (s, 3H). | 397 |
| | 5-chloro-1-methyl-4-(3- | | | |
| | (5-(trifluoromethyl)- | | | |
| | 1H-benzo[d]imidazol-2- | | | |
| | yl)-4H-1,2,4-triazol-4- | | | |
| | yl)pyridin-2(1H)-one | | | |
| Example 174 Method 1 | 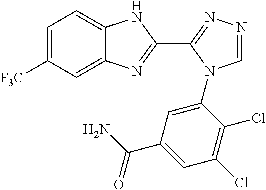 [SMILES: NC(=O)c1cc(Cl)c(Cl)c(-n2cnnc2-c2nc3cc(C(F)(F)F)ccc3[nH]2)c1] | white solid, yield: 50 % | ¹H NMR (400 MHz, MeOD) δ 8.80 (s, 1H), 8.24 (d, J = 2.2 Hz, 1H), 8.04 (d, J = 2.1 Hz, 1H), 7.75 (s, 1H), 7.60 (d, J = 8.6 Hz, 1H), 7.44 (d, J = 8.6 Hz, 1H). | 441 |
| | 3,4-dichloro-5-(3-(5- | | | |
| | (trifluoromethyl)-1H- | | | |
| | benzo[d]imidazol-2-yl)- | | | |
| | 4H-1,2,4-triazol-4- | | | |
| | yl)benzamide | | | |
| Example 175 Method 1 | 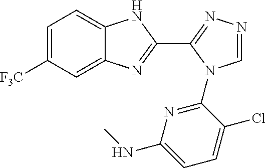 [SMILES: CNc1ccc(Cl)c(-n2cnnc2-c2nc3cc(C(F)(F)F)ccc3[nH]2)n1] | white solid, yield: 11 % | ¹H NMR (400 MHz, DMSO-d₆) δ 13.98 (s, 1H), 9.11 (s, 1H), 7.87 (s, 1H), 7.71 (dd, J = 16.9, 8.7 Hz, 2H), 7.54 (bs, 2H), 7.15 (q, J = 4.8 Hz, 1H), 6.71 (d, J = 8.9 Hz, 1H), 2.67 (d, J = 4.7 Hz, 3H). | 395 |
| | 5-Chloro-N-methyl-6- | | | |
| | (3-(5-(trifluoromethyl)- | | | |
| | 1H-benzo[d]imidazol-2- | | | |
| | yl)-4H-1,2,4-triazol-4- | | | |
| | yl)pyridin-2-amine | | | |
| Example 176 Method 1 | 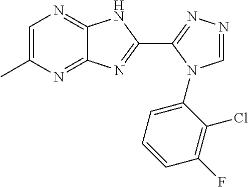 [SMILES: Cc1cnc2[nH]c(-c3nncn3-c3cccc(F)c3Cl)nc2n1] | white solid, | ¹H NMR (400 MHz, Methanol-d₄) δ 8.91 (s, 1H), 8.32 (s, 1H), 7.63- 7.48 (m, J = 3.6 Hz, 3H), 2.62 (s, 3H). | 330 |
| | 2-(4-(2-chloro-3- | | | |
| | fluorophenyl)-4H-1,2,4- | | | |
| | triazol-3-yl)-6-methyl-1H- | | | |
| | imidazo[4,5-b]pyrazine | | | |
| Example 177 Method 13 | 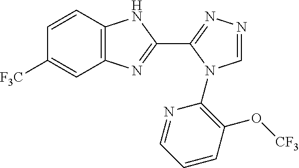 [SMILES: FC(F)(F)Oc1cccnc1-n1cnnc1-c1nc2cc(C(F)(F)F)ccc2[nH]1] | white solid, yield: 17 % | ¹H NMR (400 MHz, DMSO-d₆) δ 14.13 (s, 1H), 9.25 (s, 1H), 8.68 (dd, J = 4.8 Hz, J = 1.2 Hz, 1H), 8.24 (dt, J = 8.4 Hz, J = 1.2 Hz, 1H), 7.88 (dd, J = 8.4 Hz, J = 4.8 Hz, 1H), 7.87-7.40 (m, 3H). | 415 |
| | (trifluoromethoxy) | | | |
| | pyridin-2-yl)-4H-1,2,4- | | | |
| | triazol-3-yl)-5- | | | |
| | (trifluoromethyl)-1H- | | | |
| | benzo[d]imidazole | | | |
| Example 178 Method 1 | 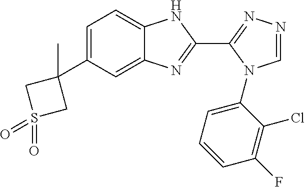 [SMILES: CC1(c2ccc3[nH]c(-c4nncn4-c4cccc(F)c4Cl)nc3c2)CS(=O)(=O)C1] | white solid, yield: 3% | ¹H NMR (400 MHz, DMSO-d₆) δ 13.51 (s, 1H), 9.04 (s, 1H), 7.75- 7.60 (m, 3H), 7.51-7.49 (m, 1H), 7.40 (s, 1H), 7.20-7.17 (m, 1H), 4.58- 4.54 (m, 2H), 4.35- 4.30 (m, 2H), 1.69 (s, 3H). | 432 |
| | 3-(2-(4-(2-chloro-3- | | | |
| | fluorophenyl)-4H-1,2,4- | | | |
| | triazol-3-yl)-1H- | | | |
| | benzo[d]imidazol-5-yl)- | | | |
| | 3-methylthietane 1,1- | | | |
| | dioxide | | | |
| Example 179 Method 1 | 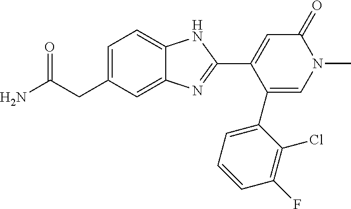 [SMILES: Cn1cc(-c2cccc(F)c2Cl)c(-c2nc3cc(CC(N)=O)ccc3[nH]2)cc1=O] | white solid, yield: 11.3% | ¹HNMR (400 MHz, DMSO-d₆) δ 12.73 (m, 1H), 7.88 (d, J = 2.8 Hz, 1H), 7.33-7.48 (m, 4H), 7.24-7.28 (m, 2H), 7.00-7.12 (m,1H), 6.95 (d, J = 7.6 Hz, 1H), 6.82 (m, 1H), 3.54 (s, 3H), 3.41 (s, 2H). | 411/413 |
| | 2-(2-(5-(2-chloro-3- | | | |
| | fluorophenyl)-1-methyl- | | | |
| | 2-oxo-1,2- | | | |
| | dihydropyridin-4-yl)- | | | |
| | 1H-benzo[d]imidazol-5- | | | |
| | yl)acetamide | | | |
| Example 180 Method 1 | 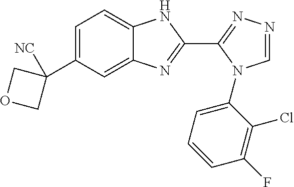 [SMILES: N#CC1(c2ccc3[nH]c(-c4nncn4-c4cccc(F)c4Cl)nc3c2)COC1] | white solid, yield: 7% | ¹H NMR (400 MHz, DMSO-d₆) δ 13.80 (s, 1H), 9.07 (s, 1H), 7.75- 7.71 (m, 1H), 7.68 (d, J = 5.6 Hz, 2H), 7.63 (dd, J = 10.6, 5.2 Hz, 2H), 7.44 (d, J = 8.2 Hz, 1H), 5.17 (d, J = 6.6 Hz, 2H), 4.93 (d, J = 6.6 Hz, 2H). | 394 |
| | 3-(2-(4-(2-chloro-3- | | | |
| | fluorophenyl)-4H-1,2,4- | | | |
| | triazol-3-yl)-1H- | | | |
| | benzo[d]imidazol-5- | | | |
| | yl)oxetane-3- | | | |
| | carbonitrile | | | |
| Example 181 Method 9 | 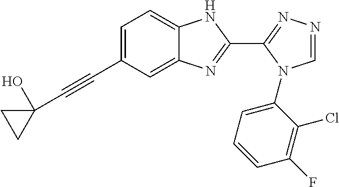 [SMILES: OC1(C#Cc2ccc3[nH]c(-c4nncn4-c4cccc(F)c4Cl)nc3c2)CC1] | white solid, yield: 2% | ¹H NMR (400 MHz, DMSO-d₆) δ 13.73 (s, 1H), 9.04 (s, 1H), 7.77 (d, J = 5.2 Hz, 2H), 7.47 (m, 2H), 7.17 (s, 1H), 6.26 (s, 1H), 1.24 (s, 4H) | 412 |
| | 1-((2-(4-(2-chloro-3,4- | | | |
| | difluorophenyl)-4H- | | | |
| | 1,2,4-triazol-3-yl)-1H- | | | |
| | benzo[d]imidazol-5- | | | |
| | yl)ethynyl)cyclopropan-1-ol | | | |
| Example 182 Method 14 mixture of stereoisomers | 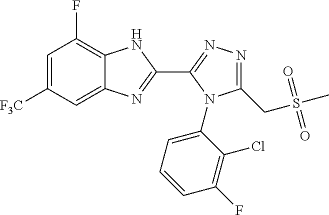 [SMILES: CS(=O)(=O)Cc1nnc(-c2nc3cc(C(F)(F)F)cc(F)c3[nH]2)n1-c1cccc(F)c1Cl] | white solid | ¹H NMR (400 MHz, DMSO-d₆) δ 14.52 (s, 1H), 7.76 (td, J = 8.7, 2.0 Hz, 1H), 7.71-7.61 (m, 3H), 7.50-7.36 (m, 1H), 4.93 (d, J = 15.1 Hz, 1H), 4.72 (d, J = 15.1 Hz, 1H), 3.20 (s, 3H) | 492 |
| | 2-(4-(2-Chloro-3- | | | |
| | fluorophenyl)-5- | | | |
| | ((methylsulfonyl)methyl)- | | | |
| | 4H-1,2,4-triazol-3- | | | |
| | yl)-7-fluoro-5- | | | |
| | (trifluoromethyl)-1H- | | | |
| | benzo[d]imidazole | | | |
| Example 183 Method 14 single unknown stereoisomer | 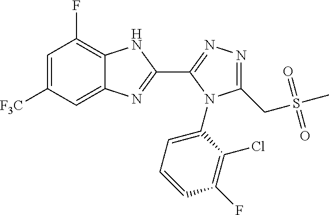 | white solid | ¹H NMR (400 MHz, DMSO) δ 14.52 (s, 1H), 7.81-7.59 (m, 4H), 7.43 (s, 1H), 4.93 (d, J = 15.1 Hz, 1H), 4.72 (d, J = 15.1 Hz, 1H), 3.20 (s, 3H) | 492 Note: CHIRAL PAK IF- 3 70% Hex (0.1% FA): 30% (ACN:DCM) RT = 1.93min |
| | 2-(4-(2-Chloro-3- | | | |
| | fluorophenyl)-5- | | | |
| | ((methylsulfonyl)methyl)- | | | |
| | 4H-1,2,4-triazol-3- | | | |
| | yl)-7-fluoro-5- | | | |
| | (trifluoromethyl)-1H- | | | |
| | benzo[d]imidazole | | | |
| Example 184 Method 14 single unknown stereoisomer | 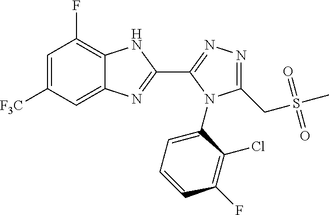 | white solid | ¹H NMR (400 MHz, DMSO-d6) δ 14.67 (d, J = 122.1 Hz, 1H), 7.85- 7.59 (m, 4H), 7.43 (s, 1H), 4.93 (d, J = 15.1 Hz, 1H), 4.72 (d, J = 15.1 Hz, 1H), 3.20 (s, 3H). | 492 Note: CHIRAL PAK IF- 3 70% Hex (0.1% FA): 30% (ACN:DCM) RT = 2.81 min |
| | 2-(4-(2-Chloro-3- | | | |
| | fluorophenyl)-5- | | | |
| | ((methylsulfonyl)methyl)- | | | |
| | 4H-1,2,4-triazol-3- | | | |
| | yl)-7-fluoro-5- | | | |
| | (trifluoromethyl)-1H- | | | |
| | benzo[d]imidazole | | | |
| Example 185 Method 14 mixture of stereoisomers | 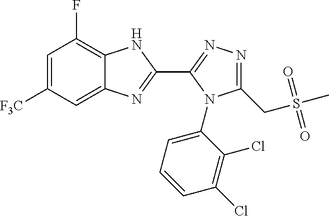 [SMILES: CS(=O)(=O)Cc1nnc(-c2nc3cc(C(F)(F)F)cc(F)c3[nH]2)n1-c1cccc(Cl)c1Cl] | white solid, yield: 42% | ¹H NMR (400 MHz, DMSO-d₆) δ 14.55 (bs, 1H), 7.95 (d, J = 8.0 Hz, 1H), 7.75 (s, J = 8.0 Hz, 1H), 7.68 (s, 1H), 7.75 (t, J = 8.0 Hz, 1H), 7.42 (d, J = 9.2 Hz, 1H), 4.95 (d, J = 15.2 Hz, 1H), 4.68 (d, J = 15.2 Hz, 1H), 3.19(s, 3H). | 508 |
| | 2-(4-(2,3- | | | |
| | dichlorophenyl)-5- | | | |
| | ((methylsulfonyl)methyl)- | | | |
| | 4H-1,2,4-triazol-3- | | | |
| | yl)-4-fluoro-6- | | | |
| | (trifluoromethyl)-1H- | | | |
| | benzo[d]imidazole | | | |
| Example 186 Method 14 single unknown stereoisomer | 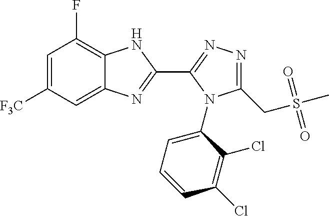 | white solid | ¹H NMR (400 MHz, DMSO) δ 14.14 (s, 1H), 7.94 (td, J = 9.2, 2.9 Hz, 1H), 7.85 (s, 1H), 7.77- 7.68 (m, 2H), 7.56 (b, 1H), 4.95 (d, J = 15.1 Hz, 1H), 4.83 (d, J = 15.1 Hz, 1H), 3.19 (s, 3H). | 508 Note: CHIRAL PAK IF- 3 70% Hex (0.1% FA): 30% (ACN:DCM) RT = 2.05min |
| | Dichlorophenyl)-5- | | | |
| | ((methylsulfonyl)methyl)- | | | |
| | 4H-1,2,4-triazol-3- | | | |
| | yl)-7-fluoro-5- | | | |
| | (trifluoromethyl)-1H- | | | |
| | benzo[d]imidazole | | | |
| Example 187 Method 14 single unknown stereoisomer | 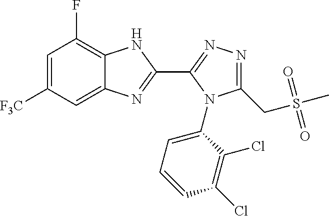 | white solid | ¹H NMR (400 MHz, DMSO) δ 14.14 (s, 1H), 7.94 (td, J = 9.2, 2.9 Hz, 1H), 7.85 (s, 1H), 7.78- 7.66 (m, 2H), 7.56 (s, 1H), 4.95 (d, J = 15.1 Hz, 1H), 4.83 (d, J = 15.1 Hz, 1H), 3.19 (s, 3H). | 508 Note: CHIRAL PAK IF-3 70% Hex (0.1% FA):30% (ACN:DCM) RT = 2.98min |
| | 2-(4-(2,3- | | | |
| | Dichlorophenyl)-5- | | | |
| | ((methylsulfonyl)methyl)- | | | |
| | 4H-1,2,4-triazol-3- | | | |
| | yl)-7-fluoro-5- | | | |
| | (trifluoromethyl)-1H- | | | |
| | benzo[d]imidazole | | | |
| Example 188 Method 10 | 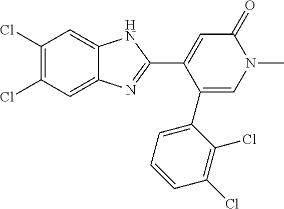 [SMILES: Cn1cc(-c2cccc(Cl)c2Cl)c(-c2nc3cc(Cl)c(Cl)cc3[nH]2)cc1=O] | brown solid, yield: 42.7% | ¹H NMR (400 MHz, DMSO-d₆) δ 13.16 (s, 1H), 7.90 (s, 1H), 7.69 (d, J = 28.5 Hz, 2H), 7.59 (dd, J = 5.9, 3.6 Hz, 1H), 7.39 (d, J = 3.6 Hz, 2H), 6.96 (s, 1H), 3.54 (s, 3H). | 438 |
| | 4-(5,6-dichloro-1H- | | | |
| | benzo[d]imidazol-2-yl)- | | | |
| | 5-(2,3-dichlorophenyl)- | | | |
| | 1-methylpyridin-2(1H)- | | | |
| | one | | | |
| Example 189 Method 13 | 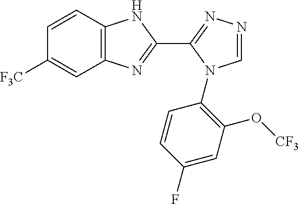 [SMILES: Fc1ccc(-n2cnnc2-c2nc3cc(C(F)(F)F)ccc3[nH]2)c(OC(F)(F)F)c1] | white solid, yield: 22% | ¹H NMR (400 MHz, DMSO-d₆) δ 14.15- 14.00 (m, 1H), 9.10 (s, 1H), 8.01-7.97 (m, 1H), 7.86-7.80 (m, 1H), 7.77- 7.66 (m, 2H), 7.64- 7.44 (m, 2H). | 432 |
| | 2-(4-(4-fluoro-2- | | | |
| | (trifluoromethoxy)phenyl)- | | | |
| | 4H-1,2,4-triazol-3- | | | |
| | yl)-5-(trifluoromethyl)- | | | |
| | 1H-benzo[d]imidazole | | | |
| Example 190 Method 13 | 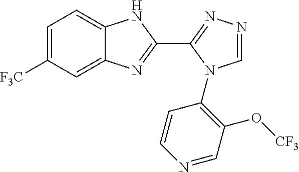 [SMILES: FC(F)(F)Oc1cnccc1-n1cnnc1-c1nc2cc(C(F)(F)F)ccc2[nH]1] | white solid, yield 42% | ¹H NMR (400 MHz, DMSO-d₆) δ 9.01 (s, 1H), 8.87-8.82 (m, 2H), 7.97-7.80 (m, 2H), 7.71 (d, J = 8.4 Hz, 1H), 7.56 (d, J = 8.4 Hz, 1H). | 415 |
| | (trifluoromethoxy) | | | |
| | pyridin-4-yl)-4H-1,2,4- | | | |
| | triazol-3-yl)-5- | | | |
| | (trifluoromethyl)-1H- | | | |
| | benzo[d]imidazole | | | |
| Example 191 Method 9 | 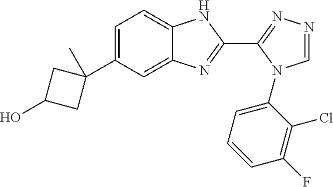 [SMILES: CC1(c2ccc3[nH]c(-c4nncn4-c4cccc(F)c4Cl)nc3c2)CC(O)C1] | white solid, yield: 53.7% | ¹H NMR (400 MHz, Methanol-d₄) δ 8.85 (s, 1H), 7.63-7.44 (m, 4H), 7.38-6.96 (m, 2H), 4.21 (m, 1H), 2.85 (m, 1H), 2.56 (m, 1H), 2.22 (m, 1H), 2.08 (m, 1H), 1.43 (d, J = 17.8 Hz, 3H). | 398 |
| | 3-(2-(4-(2-chloro-3- | | | |
| | fluorophenyl)-4H-1,2,4- | | | |
| | triazol-3-yl)-1H- | | | |
| | benzo[d]imidazol-5-yl)- | | | |
| | 3-methylcyclobutan-1-ol | | | |
| Example 192 Method 14 | 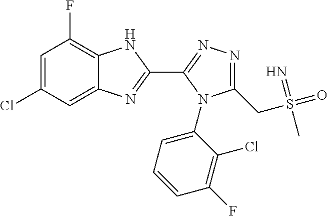 [SMILES: CS(=N)(=O)Cc1nnc(-c2nc3cc(Cl)cc(F)c3[nH]2)n1-c1cccc(F)c1Cl] | white solid, yield: 50.6% | ¹H NMR (400 MHz, Methanol-d₄) δ 8.50 (s, 1H), 7.60 (m, 2H), 7.39 (m, 1H), 7.02 (s, 1H), 5.19 (s, 2H), 3.22 (s, 3H). | 457 |
| | ((4-(2-chloro-3- | | | |
| | fluorophenyl)-5-(5- | | | |
| | chloro-7-fluoro-1H- | | | |
| | benzo[d]imidazol-2-yl)- | | | |
| | 4H-1,2,4-triazol-3- | | | |
| | yl)methyl)(imino)(methyl)- | | | |
| | 16-sulfanone | | | |
| Example 193 Method 14 | 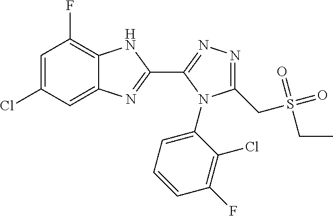 [SMILES: CCS(=O)(=O)Cc1nnc(-c2nc3cc(Cl)cc(F)c3[nH]2)n1-c1cccc(F)c1Cl] | white solid, yield: 56% | ¹H NMR (400 MHz, DMSO-d₆) δ 7.82-7.57 (m, 3H), 7.39 (s, 1H), 7.19 (d, J = 10.4 Hz, 1H), 4.89 (d, J = 15.2 Hz, 1H), 4.64 (d, J = 15.2 Hz, 1H), 1.31- 1.21 (m, 3H). | 471 |
| | 5-chloro-2-(4-(2-chloro-3-fluorophenyl)-5- | | | |
| | ((ethylsulfonyl)methyl)- | | | |
| | 4H-1,2,4-triazol-3-yl)- | | | |
| | 7-fluoro-1H- | | | |
| | benzo[d]imidazole | | | |
| Example 194 Method 14 | 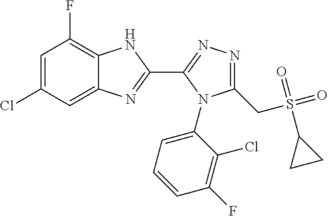 [SMILES: O=S(=O)(Cc1nnc(-c2nc3cc(Cl)cc(F)c3[nH]2)n1-c1cccc(F)c1Cl)C1CC1] | white solid, yield: 47% | ¹H NMR (400 MHz, DMSO-d₆) δ 14.16 (s, 1H), 7.77-7.70 (m, 1H), 7.69-7.63 (m, 2H), 7.40 (d, J = 1.7 Hz, 1H), 7.18 (d, J = 10.4 Hz, 1H), 4.90 (d, J = 15.1 Hz, 1H), 4.75 (d, J = 15.2 Hz, 1H), 3.02- 2.84 (m, 1H), 1.16-1.03 (m, 2H), 1.02-0.87 (m, 2H). | 484 |
| | 5-chloro-2-(4-(2-chloro- | | | |
| | 3-fluorophenyl)-5- | | | |
| | ((cyclopropylsulfonyl) | | | |
| | methyl)-4H-1,2,4- | | | |
| | triazol-3-yl)-7-fluoro- | | | |
| | 1H-benzo[d]imidazole | | | |
| Example 195 Method 14 mixture of stereoisomers | 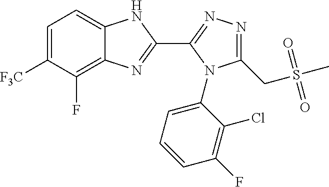 [SMILES: CS(=O)(=O)Cc1nnc(-c2nc3c(F)c(C(F)(F)F)ccc3[nH]2)n1-c1cccc(F)c1Cl] | white solid, yield: 65% | ¹H NMR (400 MHz, DMSO-d₆) δ 14.44 (s, 1H), 7.75-7.49 (m, 5H), 4.93 (d, J = 15.2 Hz, 1H), 4.71 (d, J = 14.8 Hz, 1H), 3.19 (s, 3H). | 492 |
| | 2-(4-(2-chloro-3- | | | |
| | fluorophenyl)-5- | | | |
| | ((methylsulfonyl)methyl)- | | | |
| | 4H-1,2,4-triazol-3- | | | |
| | yl)-4-fluoro-5- | | | |
| | (trifluoromethyl)-1H- | | | |
| | benzo[d]imidazole | | | |
| Example 196 Method 14 single unknown stereoisomer | 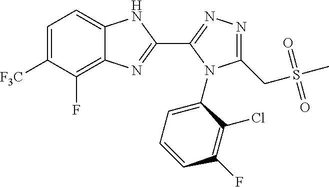 | white solid, | ¹H NMR (400 MHz, DMSO-d₆): δ 14.44 (s, 1H), 7.77-7.49 (m, 5H), 4.93 (d, J = 15.2 Hz, 1H), 4.71 (d, J = 15.2 Hz, 1H), 3.19 (s, 3H) | 492 |
| | 2-(4-(2-chloro-3- | | | |
| | fluorophenyl)-5- | | | |
| | ((methylsulfonyl)methyl)- | | | |
| | 4H-1,2,4-triazol-3- | | | |
| | yl)-4-fluoro-5- | | | |
| | (trifluoromethyl)-1H- | | | |
| | benzo[d]imidazole | | | |
| Example 197 Method 14 single unknown stereoisomer | 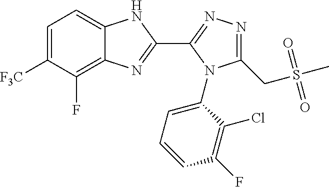 | white solid | ¹H NMR (400 MHz, DMSO-d₆): δ 14.42 (s, 1H), 7.76-7.51 (m, 5H), 4.93 (d, J = 15.2 Hz, 1H), 4.71 (d, J = 15.2 Hz, 1H), 3.19 (s, 3H). | 492 |
| | 2-(4-(2-chloro-3- | | | |
| | fluorophenyl)-5- | | | |
| | ((methylsulfonyl)methyl)- | | | |
| | 4H-1,2,4-triazol-3- | | | |
| | yl)-4-fluoro-5- | | | |
| | (trifluoromethyl)-1H- | | | |
| | benzo[d]imidazole | | | |
| Example 198 Method 1 | 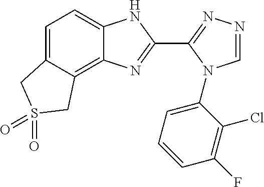 [SMILES: O=S1(=O)Cc2ccc3[nH]c(-c4nncn4-c4cccc(F)c4Cl)nc3c2C1] | white solid, yield: 7% | | 403 |
| | 2-(4-(2-chloro-3- | | | |
| | fluorophenyl)-4H-1,2,4- | | | |
| | triazol-3-yl)-6,8- | | | |
| | dihydro-3H- | | | |
| | thieno[3',4':3,4]benzo[1, | | | |
| | 2-d]imidazole 7,7- | | | |
| | dioxide | | | |
| Example 199 Method 10 | 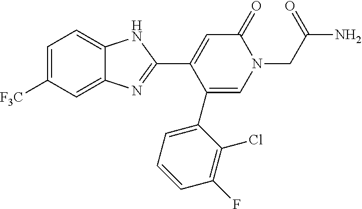 [SMILES: NC(=O)Cn1cc(-c2cccc(F)c2Cl)c(-c2nc3cc(C(F)(F)F)ccc3[nH]2)cc1=O] | white solid, yield: 6% | ¹H NMR (400 MHz, DMSO-d₆) 13.31 (s, 1H), 7.84 (s, 1H), 7.76-7.71 (m, 2H), 7.46-7.36 (m, 4H), 7.27-7.26 (m, 2H), 6.98 (s, 1H), 4.63 (d, J = 5.2 Hz, 2H). | 464/466 |
| | 2-(5-(2-chloro-3- | | | |
| | fluorophenyl)-2-oxo-4- | | | |
| | (5-(trifluoromethyl)- | | | |
| | 1H-benzo[d]imidazol-2- | | | |
| | yl)pyridin-1(2H)- | | | |
| | yl)acetamide | | | |
| Example 200 Method 13 | 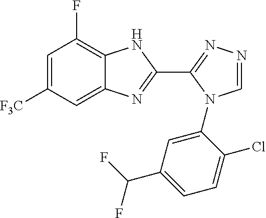 [SMILES: Fc1cc(C(F)(F)F)cc2nc(-c3nncn3-c3cc(C(F)F)ccc3Cl)[nH]c12] | white solid, yield: 50% | ¹H NMR (400 MHz, DMSO-d₆) δ 14.64 (d, J = 140.8 Hz, 1H), 9.16 (s, 1H), 8.10 (s, 1H), 7.90 (dd, J = 22.2, 8.3 Hz, 2H), 7.68 (s, 1H), 7.45 (t, J = 29.0 Hz, 1H), 7.16 (t, J = 55.2 Hz, 1H). | 431 |
| | 2-(4-(2-chloro-5- | | | |
| | (difluoromethyl)phenyl)- | | | |
| | 4H-1,2,4-triazol-3-yl)- | | | |
| | 7-fluoro-5- | | | |
| | (trifluoromethyl)-1H- | | | |
| | benzo[d]imidazole | | | |
| Example 201 Method 13 | 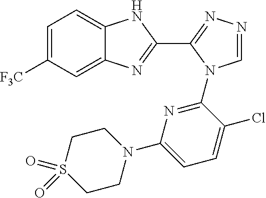 [SMILES: O=S1(=O)CCN(c2ccc(Cl)c(-n3cnnc3-c3nc4cc(C(F)(F)F)ccc4[nH]3)n2)CC1] | white solid, yield: 8% | 1H NMR (400 MHz, DMSO-d₆) δ 9.18 (s, 1H), 8.01 (d, J = 8.8 Hz, 1H), 7.86 (s, 1H), 7.71 (d, J = 8.4 Hz, 1H), 7.53 (d, J = 8.8 Hz, 1H), 7.37 (d, J = 9.2 Hz, 1H), 4.02 (t, J = 5.2 Hz, 4H), 3.10 (t, J = 5.2 Hz, 4H). | 498 |
| | 4-(5-chloro-6-(3-(5- | | | |
| | (trifluoromethyl)-1H- | | | |
| | benzo[d]imidazol-2-yl)- | | | |
| | 4H-1,2,4-triazol-4- | | | |
| | yl)pyridin-2- | | | |
| | yl)thiomorpholine 1,1- | | | |
| | dioxide | | | |
| Example 202 Method 13 | 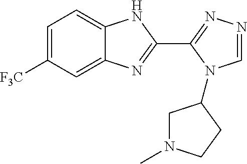 [SMILES: CN1CCC(n2cnnc2-c2nc3cc(C(F)(F)F)ccc3[nH]2)C1] | white solid, yield: 37% | ¹H NMR (400 MHz, DMSO-d₆) δ 8.94 (s, 1H), 8.14 (s, 1H), 8.03 (s, 1H), 7.82 (s, 1H), 7.61 (d, J = 8.4 Hz, 1H), 6.09-5.98 (m, 1H), 3.06 (td, J = 8.6, 4.1 Hz, 1H), 2.93 (dd, J = 10.4, 2.3 Hz, 1H), 2.74 (dd, J = 10.3, 6.3 Hz, 1H), 2.63- | 337 |
| | | | 2.53 (m, 1H), 2.38 (d, | |
| | 2-(4-(1- | | J = 8.7 Hz, 1H), 2.34 (s, | |
| | methylpyrrolidin-3-yl)- | | 3H), 2.10-1.95 (m, | |
| | 4H-1,2,4-triazol-3-yl)- | | 1H). | |
| | 5-(trifluoromethyl)-1H- | | | |
| | benzo[d]imidazole | | | |
| Example 203 Method 14 | 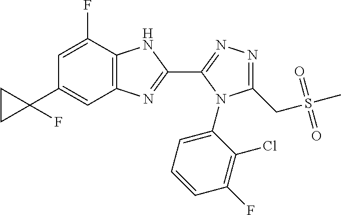 [SMILES: CS(=O)(=O)Cc1nnc(-c2nc3cc(C4(F)CC4)cc(F)c3[nH]2)n1-c1cccc(F)c1Cl] | white solid, yield: 61% | ¹H NMR (400 MHz, Chloroform-d) δ 11.96 (s, 1H), 7.74-7.46 (m, 4H), 6.64 (d, J = 11.2 Hz, 1H), 4.64 (t, J = 14.9 Hz, 1H), 4.05 (t, J = 15.5 Hz, 1H), 3.27 (s, 3H), 1.53-1.42 (m, 2H), 1.17- 1.01 (m, 2H). | 482 |
| | 2-(4-(2-chloro-3- | | | |
| | fluorophenyl)-5- | | | |
| | ((methylsulfonyl)methyl)- | | | |
| | 4H-1,2,4-triazol-3- | | | |
| | yl)-7-fluoro-5-(1- | | | |
| | fluorocyclopropyl)-1H- | | | |
| | benzo[d]imidazole | | | |
| Example 204 Method 9 | 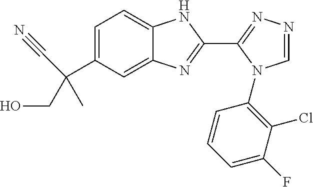 [SMILES: CC(C#N)(CO)c1ccc2[nH]c(-c3nncn3-c3cccc(F)c3Cl)nc2c1] | white solid, yield: 25% | ¹H NMR (400 MHz, DMSO-d₆) δ 9.05 (s, 1H), 7.75-7.70 (m, 1H), 7.65 (dt, J = 8.0, 5.8 Hz, 2H), 7.57-7.51 (m, 2H), 7.35 (d, J = 8.2 Hz, 1H), 3.68 (dd, J = 22.8, 10.8 Hz, 2H), 1.66 (s, 3H). | 397 |
| | 2-(2-(4-(2-chloro-3- | | | |
| | fluorophenyl)-4H-1,2,4- | | | |
| | triazol-3-yl)-1H- | | | |
| | benzo[d]imidazol-5-yl)- | | | |
| | 3-hydroxy-2- | | | |
| | methylpropanenitrile | | | |
| Example 205 Method 13 | 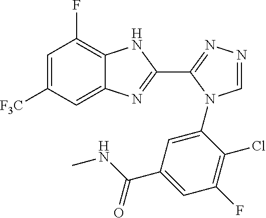 [SMILES: CNC(=O)c1cc(F)c(Cl)c(-n2cnnc2-c2nc3cc(C(F)(F)F)cc(F)c3[nH]2)c1] | white solid, yield: 58% | ¹H NMR (400 MHz, DMSO-d₆) δ 9.11 (s, 1H), 8.75-8.74 (m, 1H), 8.14-8.08 (m, 2H), 7.66 (s, 1H), 7.37-7.35 (m, 1H), 2.80 (d, J = 4.4 Hz, 3H). | 457 |
| | 4-chloro-3-fluoro-5-(3- | | | |
| | (7-fluoro-5- | | | |
| | (trifluoromethyl)-1H- | | | |
| | benzo[d]imidazol-2-yl)- | | | |
| | 4H-1,2,4-triazol-4-yl)- | | | |
| | N-methylbenzamide | | | |
| Example 206 Method 13 | 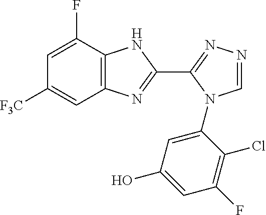 [SMILES: Oc1cc(F)c(Cl)c(-n2cnnc2-c2nc3cc(C(F)(F)F)cc(F)c3[nH]2)c1] | white solid, yield: 14% | ¹H NMR (400 MHz, DMSO-d₆) δ 14.45 (s, 1H), 10.76 (s, 1H), 9.09 (s, 1H), 7.71 (s, 1H), 7.45 (m, 1H), 7.05 (s, 2H). | 416 |
| | 4-chloro-3-fluoro-5-(3- | | | |
| | (7-fluoro-5- | | | |
| | (trifluoromethyl)-1H- | | | |
| | benzo[d]imidazol-2-yl)- | | | |
| | 4H-1,2,4-triazol-4- | | | |
| | yl)phenol | | | |
| Example 207 Method 13 | 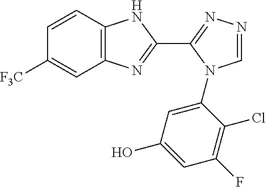 [SMILES: Oc1cc(F)c(Cl)c(-n2cnnc2-c2nc3cc(C(F)(F)F)ccc3[nH]2)c1] | white solid, yield: 11% | ¹H NMR (400 MHz, DMSO-d₆) δ 14.02 (s, 1H), 10.74 (s, 1H), 9.06 (s, 1H), 7.91-7.48 (m, 3H), 7.08-7.00 (m, 2H). | 398 |
| | 4-chloro-3-fluoro-5-(3- | | | |
| | (5-(trifluoromethyl)- | | | |
| | 1H-benzo[d]imidazol-2- | | | |
| | yl)-4H-1,2,4-triazol-4- | | | |
| | yl)phenol | | | |
| Example 208 Method 13 | 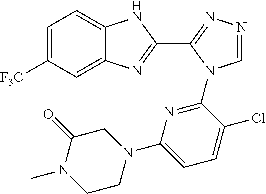 [SMILES: CN1CCN(c2ccc(Cl)c(-n3cnnc3-c3nc4cc(C(F)(F)F)ccc4[nH]3)n2)CC1=O] | white solid, yield: 24.6% | ¹H NMR (400 MHz, DMSO-d₆) δ 9.18 (s, 1H), 7.94 (d, J = 9.2 Hz, 1H), 7.87 (s, 1H), 7.73 (d, J = 8.4 Hz, 1H), 7.55 (d, J = 9.2 Hz, 1H), 7.15 (d, J = 9.2 Hz, 1H), 4.05 (s, 2H), 3.76 (t, J = 5.6 Hz, 2H), 2.84 (s, 3H), 2.01-1.92 (m, 2H). | 477 |
| | 4-(5-chloro-6-(3-(5- | | | |
| | (trifluoromethyl)-1H- | | | |
| | benzo[d]imidazol-2-yl)- | | | |
| | 4H-1,2,4-triazol-4- | | | |
| | yl)pyridin-2-yl)-1- | | | |
| | methylpiperazin-2-one | | | |
| Example 209 Method 13 | 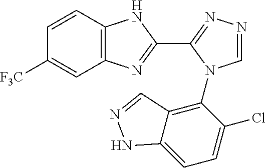 [SMILES: FC(F)(F)c1ccc2[nH]c(-c3nncn3-c3c(Cl)ccc4[nH]ncc34)nc2c1] | white solid, yield: 25.24% | ¹H NMR (400 MHz, MeOD) δ 9.01 (s, 1H), 7.84-7.77 (m, 3H), 7.65 (d, J = 8.0 Hz, 1H), 7.60 (d, J = 8.8 Hz, 1H), 7.50 (d, J = 8.4 Hz, 1H). | 403/405 |
| | 5-chloro-4-(3-(5- | | | |
| | (trifluoromethyl)-1H- | | | |
| | benzo[d]imidazol-2-yl)- | | | |
| | 4H-1,2,4-triazol-4-yl)- | | | |
| | 1H-indazole | | | |
| Example 210 Method 13 | 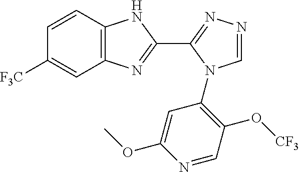 [SMILES: COc1cc(-n2cnnc2-c2nc3cc(C(F)(F)F)ccc3[nH]2)c(OC(F)(F)F)cn1] | white solid, yield: 13% | ¹H NMR (400 MHz, Methanol-d₄) δ 8.98 (s, 1H), 8.36 (d, J = 1.6 Hz, 1H), 7.89 (s, 1H), 7.74 (d, J = 8.4 Hz, 1H), 7.57 (d, J = 8.4 Hz, 1H), 7.32 (s, 1H), 4.06 (s, 3H) | 445 |
| | 2-(4-(2-methoxy-5- | | | |
| | (trifluoromethoxy)pyrid | | | |
| | in-4-yl)-4H-1,2,4- | | | |
| | triazol-3-yl)-5- | | | |
| | (trifluoromethyl)-1H- | | | |
| | benzo[d]imidazole | | | |
| Example 211 Method 13 | 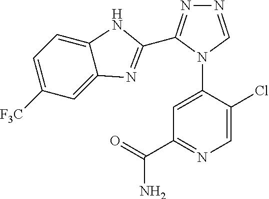 [SMILES: NC(=O)c1cc(-n2cnnc2-c2nc3cc(C(F)(F)F)ccc3[nH]2)c(Cl)cn1] | white solid, yield: 16% | ¹H NMR (400 MHz, DMSO-d₆) δ 14.04 (s, 1H), 9.11 (s, 1H), 9.01 (s, 1H), 8.43 (s, 1H), 8.36 (s, 1H), 7.93 (s, 1H), 7.82 (s, 1H), 7.69 (d, J = 8.4 Hz, 1H), 7.52 (d, J = 8.4 Hz, 1H). | 408 |
| | 5-chloro-4-(3-(5- | | | |
| | (trifluoromethyl)-1H- | | | |
| | benzo[d]imidazol-2-yl)- | | | |
| | 4H-1,2,4-triazol-4- | | | |
| | yl)picolinamide | | | |
| Example 212 Method 13 | 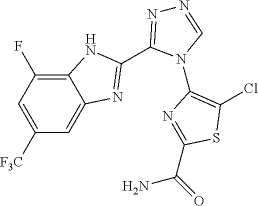 [SMILES: NC(=O)c1nc(-n2cnnc2-c2nc3cc(C(F)(F)F)cc(F)c3[nH]2)c(Cl)s1] | white solid, yield: 27% | ¹H NMR (400 MHz, DMSO-d₆) δ 9.17 (s, 1H), 8.62 (s, 1H), 8.21 (s, 1H), 7.70 (s, 1H), 7.39 (d, J = 10.0 Hz, 1H). | 432 |
| | 4-chloro-5-(3-(7-fluoro- | | | |
| | 5-(trifluoromethyl)-1H- | | | |
| | benzo[d]imidazol-2-yl)- | | | |
| | 4H-1,2,4-triazol-4- | | | |
| | yl)thiazole-2- | | | |
| | carboxamide | | | |
| Example 213 Method 13 | 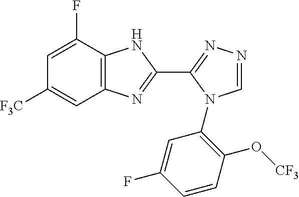 [SMILES: Fc1ccc(OC(F)(F)F)c(-n2cnnc2-c2nc3cc(C(F)(F)F)cc(F)c3[nH]2)c1] | solid, yield: 17% | ¹H NMR (400 MHz, DMSO-d₆) δ 14.48 (s, 1H), 9.18 (s, 1H), 8.00 (d, J = 8.4, 3.2 Hz, 1H), 7.76-7.64 (m, 3H), 7.44- 7.42 (m, 1H). | 449 |
| | 7-fluoro-2-(4-(5-fluoro-2- | | | |
| | (trifluoromethoxy)phenyl)- | | | |
| | 4H-1,2,4-triazol-3- | | | |
| | yl)-5-(trifluoromethyl)- | | | |
| | 1H-benzo[d]imidazole | | | |
| Example 214 Method 13 | 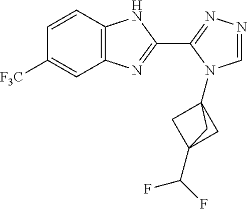 [SMILES: FC(F)C12CC(n3cnnc3-c3nc4cc(C(F)(F)F)ccc4[nH]3)(C1)C2] | white solid, | ¹H NMR (400 MHz, DMSO-d₆) δ 13.91 (b, 1H), 8.88 (s, 1H), 8.04 (s, 1H), 7.90-7.77 (m, 1H), 7.62 (dd, J = 8.5, 1.8 Hz, 1H), 6.36 (t, J = 56.1 Hz, 1H), 2.65 (s, 6H). | 369 |
| | 2-(4-(3- | | | |
| | (difluoromethyl)bicyclo | | | |
| | [1.1.1]pentan-1-yl)-4H- | | | |
| | 1,2,4-triazol-3-yl)-5- | | | |
| | (trifluoromethyl)-1H- | | | |
| | benzo[d]imidazole | | | |
| Example 215 Method 13 | 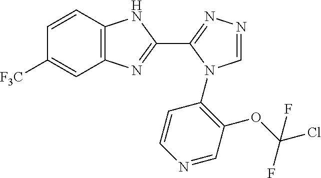 [SMILES: FC(F)(Cl)Oc1cnccc1-n1cnnc1-c1nc2cc(C(F)(F)F)ccc2[nH]1] | white solid, yield: 61% | ¹H NMR (400 MHz, Methanol-d₄) δ 9.02 (d, 1H), 8.89-8.77 (m, 2H), 7.98-7.90 (m, 1H), 7.87-7.80 (m, 1H), 7.76-7.66 (m, 1H), 7.61-7.49 (m, 1H). | 431 |
| | 2-(4-(3- | | | |
| | (chlorodifluoromethoxy | | | |
| | )pyridin-4-yl)-4H-1,2,4- | | | |
| | triazol-3-yl)-5- | | | |
| | (trifluoromethyl)-1H- | | | |
| | benzo[d]imidazole | | | |
| Example 216 Method 13 | 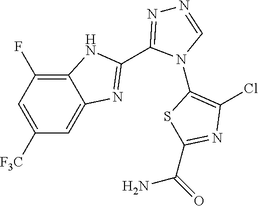 [SMILES: NC(=O)c1nc(Cl)c(-n2cnnc2-c2nc3cc(C(F)(F)F)cc(F)c3[nH]2)s1] | white solid, yield: 27% | ¹H NMR (400 MHz, DMSO-d₆) δ 9.17 (s, 1H), 8.62 (s, 1H), 8.21 (s, 1H), 7.70 (s, 1H), 7.39 (d, J = 10.0 Hz, 1H). | 432 |
| | 4-chloro-5-(3-(7-fluoro- | | | |
| | 5-(trifluoromethyl)-1H- | | | |
| | benzo[d]imidazol-2-yl)- | | | |
| | 4H-1,2,4-triazol-4- | | | |
| | yl)thiazole-2- | | | |
| | carboxamide | | | |
| Example 217 Method 13 | 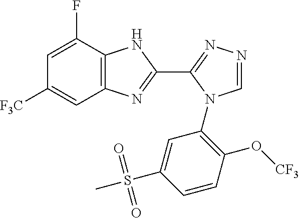 [SMILES: CS(=O)(=O)c1ccc(OC(F)(F)F)c(-n2cnnc2-c2nc3cc(C(F)(F)F)cc(F)c3[nH]2)c1] | white solid, yield: 54% | ¹H NMR (400 MHz, DMSO-d₆) δ 14.50 (s, 1H), 9.18 (s, 1H), 8.56 (d, J = 2.0 Hz, 1H), 8.30 (dd, J = 8.8, 2.0 Hz, 1H), 7.95 (dd, J = 9.2, 2.0 Hz, 1H), 7.70 (s, 1H), 7.45- 7.43 (m, 1H), 3.44 (s, 3H). | 509 |
| | 7-fluoro-2-(4-(5- | | | |
| | (methylsulfonyl)-2- | | | |
| | (trifluoromethoxy) | | | |
| | phenyl)-4H-1,2,4-triazol-3- | | | |
| | yl)-5-(trifluoromethyl)- | | | |
| | 1H-benzo[d]imidazole | | | |
| Example 218 Method 13 | 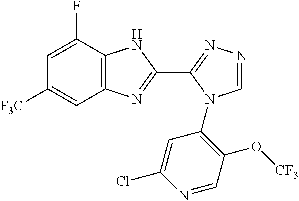 [SMILES: Fc1cc(C(F)(F)F)cc2nc(-c3nncn3-c3cc(Cl)ncc3OC(F)(F)F)[nH]c12] | white solid, yield: 33% | ¹H NMR (400 MHz, DMSO-d₆) δ 14.62 (s, 1H), 9.21 (s, 1H), 8.89 (d, J = 1.2 Hz, 1H), 8.38 (s, 1H), 7.72 (s, 1H), 7.47-7.46 (m, 1H). | 466 |
| | 2-(4-(2-chloro-5- | | | |
| | (trifluoromethoxy) | | | |
| | pyridin-4-yl)-4H-1,2,4- | | | |
| | triazol-3-yl)-7-fluoro-5- | | | |
| | (trifluoromethyl)-1H- | | | |
| | benzo[d]imidazole | | | |
| Example 219 Method 13 | 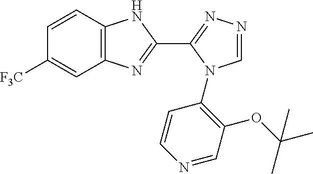 [SMILES: CC(C)(C)Oc1cnccc1-n1cnnc1-c1nc2cc(C(F)(F)F)ccc2[nH]1] | white solid, yield: 36.7% | ¹H NMR (400 MHz, DMSO-d₆) 9.06 (s, 1H), 8.57 (s, 1H), 8.50 (d, J = 5.2 Hz, 1H), 7.87 (d, J = 1.6 Hz, 1H), 7.78 (d, J = 5.2 Hz, 1H), 7.73 (d, J = 8.4 Hz, 1H), 7.60 -7.42 (m, 1H), 0.99 (s, 9H). | 404 |
| | 2-(4-(3-(tert- | | | |
| | butoxy)pyridin-4-yl)- | | | |
| | 4H-1,2,4-triazol-3-yl)- | | | |
| | 5-(trifluoromethyl)-1H- | | | |
| | benzo[d]imidazole | | | |
| Example 220 Method 13 | 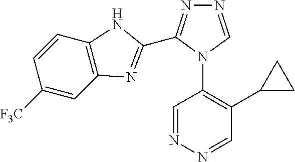 [SMILES: FC(F)(F)c1ccc2[nH]c(-c3nncn3-c3cnncc3C3CC3)nc2c1] | white solid, yield: 58% | ¹H NMR (400 MHz, DMSO-d₆) δ 14.11 (d, J = 10.3 Hz, 1H), 9.34 (s, 1H), 9.20-9.16 (m, 1H), 9.15-9.10 (m, 1H), 7.87-7.80 (m, 1H), 7.77-7.65 (m, 1H), 7.62-7.46 (m, 1H), 1.58-1.47 (m, 1H), 0.97-0.81 (m, 4H). | 372 |
| | 2-(4-(5- | | | |
| | cyclopropylpyridazin-4- | | | |
| | yl)-4H-1,2,4-triazol-3- | | | |
| | yl)-5-(trifluoromethyl)- | | | |
| | 1H-benzo[d]imidazole | | | |
| Example 221 Method 13 | 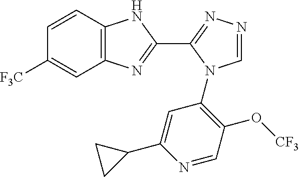 [SMILES: FC(F)(F)Oc1cnc(C2CC2)cc1-n1cnnc1-c1nc2cc(C(F)(F)F)ccc2[nH]1] | white solid, yield: 21% | ¹H NMR (400 MHz, DMSO-d₆) δ 14.17 (d, J = 13.0 Hz, 1H), 9.22 (s, 1H), 8.73 (s, 1H), 7.96 (d, J = 2.0 Hz, 1H), 7.87 (d, J = 22.0 Hz, 1H), 7.74 (t, J = 7.2 Hz, 1H), 7.56 (dd, J = 50.0, 8.8 Hz, 1H), 2.30-2.23 (m, 1H), 1.14- 1.01 (m, 4H). | 454 |
| | 2-(4-(2-cyclopropyl-5- | | | |
| | (trifluoromethoxy)pyrid | | | |
| | in-4-yl)-4H-1,2,4- | | | |
| | triazol-3-yl)-5- | | | |
| | (trifluoromethyl)-1H- | | | |
| | benzo[d]imidazole | | | |
| Example 222 Method 13 | 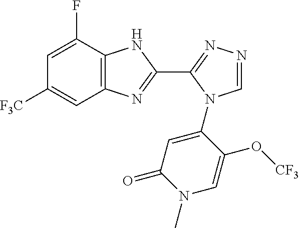 [SMILES: Cn1cc(OC(F)(F)F)c(-n2cnnc2-c2nc3cc(C(F)(F)F)cc(F)c3[nH]2)cc1=O] | white solid, yield: 29% | ¹HNMR (400 MHz, DMSO-d₆) δ 14.57 (s, 1H), 9.15 (s, 1H), 8.44 (s, 1H), 7.32-7.48 (m, 2H), 7.07 (s, 1H), 3.56 (s, 3H). | 463 |
| | 4-(3-(7-fluoro-5- | | | |
| | (trifluoromethyl)-1H- | | | |
| | benzo[d]imidazol-2-yl)- | | | |
| | 4H-1,2,4-triazol-4-yl)- | | | |
| | 1-methyl-5- | | | |
| | (trifluoromethoxy)pyridin- | | | |
| | 2(1H)-one | | | |
| Example 223 Method 9 | 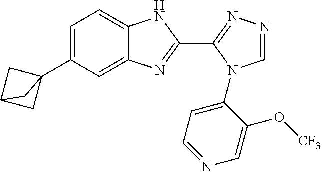 [SMILES: FC(F)(F)Oc1cnccc1-n1cnnc1-c1nc2cc(C34CC(C3)C4)ccc2[nH]1] | white solid, yield: 12% | ¹H NMR (400 MHz, Methanol-d₄) δ 8.97 (s, 1H), 8.83 (d, J = 5.2 Hz, 1H), 8.81 (d, J = 1.6 Hz, 1H), 7.88 (d, J = 5.2 Hz, 1H), 7.58-7.01 (m, 3H), 2.55 (s, 1H), 2.12 (s, 6H). | 413 |
| | 5-(bicyclo[1.1.1 ]pentan- | | | |
| | 1-yl)-2-(4-(3- | | | |
| | (trifluoromethoxy) | | | |
| | pyridin-4-yl)-4H-1,2,4- | | | |
| | triazol-3-yl)-1H- | | | |
| | benzo[d]imidazole | | | |
| Example 224 Method 9 | 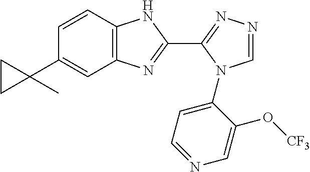 [SMILES: CC1(c2ccc3[nH]c(-c4nncn4-c4ccncc4OC(F)(F)F)nc3c2)CC1] | white solid, yield: 36.2% | ¹H NMR (400 MHz, Methanol-d₄) δ 8.96 (s, 1H), 8.88-8.76 (m, 2H), 7.88 (m, 1H), 7.55- 7.35 (m, 2H), 7.24 (m, 1H), 1.43 (s, 3H), 0.93- 0.86 (m, 2H), 0.79-0.71 (m, 2H). | 401 |
| | 5-(1- | | | |
| | methylcyclopropyl)-2- | | | |
| | (4-(3- | | | |
| | (trifluoromethoxy) | | | |
| | pyridin-4-yl)-4H-1,2,4- | | | |
| | triazol-3-yl)-1H- | | | |
| | benzo[d]imidazole | | | |
| Example 225 Method 13 | 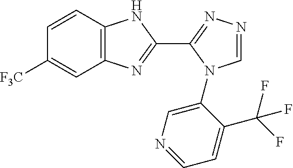 [SMILES: FC(F)(F)c1ccc2[nH]c(-c3nncn3-c3cnccc3C(F)(F)F)nc2c1] | white solid, | ¹H NMR (400 MHz, DMSO-d₆) δ 14.11 (b, 1H), 9.18 (s, 1H), 9.16 9.07 (m, 2H), 8.10 (d, J = 5.1 Hz, 1H), 7.77 (s, 1H), 7.67 (d, J = 8.5 Hz, 1H), 7.53 (d, J = 8.6 Hz, 1H). | 399 |
| | 5-(Trifluoromethyl)-2- | | | |
| | (4-(4- | | | |
| | (trifluoromethyl)pyridin- | | | |
| | 3-yl)-4H-1,2,4-triazol- | | | |
| | 3-yl)-1H- | | | |
| | benzo[d]imidazole | | | |
| Example 226 Method 13 | 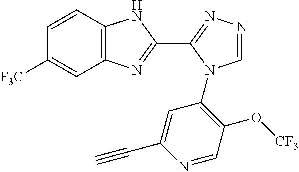 [SMILES: C#Cc1cc(-n2cnnc2-c2nc3cc(C(F)(F)F)ccc3[nH]2)c(OC(F)(F)F)cn1] | white solid, yield: 30% | ¹HNMR (400 MHz, DMSO-d₆) 14.07 (s, 1H), 9.17 (s, 1H), 8.95 (d, J = 1.2 Hz, 1H), 8.31 (s, 1H), 7.85 (s, 1H), 7.73- 7.71 (m, 1H), 7.56-7.54 (m, 1H), 4.66 (s, 1H). | 439 |
| | 2-(4-(2-ethynyl-5- | | | |
| | (trifluoromethoxy)pyridin- | | | |
| | 4-yl)-4H-1,2,4- | | | |
| | triazol-3-yl)-5- | | | |
| | (trifluoromethyl)-1H- | | | |
| | benzo[d]imidazole | | | |
| Example 227 Method 13 | 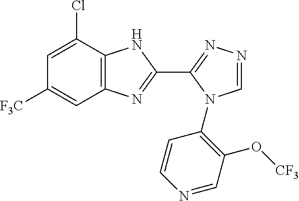 [SMILES: FC(F)(F)Oc1cnccc1-n1cnnc1-c1nc2cc(C(F)(F)F)cc(Cl)c2[nH]1] | white solid, yield: 6% | ¹H NMR (400 MHz, DMSO-d₆) δ 14.58 (s, 1H), 9.25 (s, 1H), 8.97 (s, 1H), 8.90 (d, J = 5.1 Hz, 1H), 8.06 (d, J = 5.1 Hz, 1H), 7.82 (s, 1H), 7.65 (s, 1H). | 449 |
| | 7-chloro-2-(4-(3- | | | |
| | (trifluoromethoxy)pyridin- | | | |
| | 4-yl)-4H-1,2,4- | | | |
| | triazol-3-yl)-5- | | | |
| | (trifluoromethyl)-1H- | | | |
| | benzo[d]imidazole | | | |
| Example 228 Method 13 | 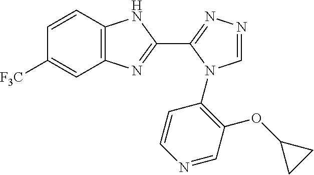 [SMILES: FC(F)(F)c1ccc2[nH]c(-c3nncn3-c3ccncc3OC3CC3)nc2c1] | white solid, yield: 34.8 % | ¹H NMR (400 MHz, MeOD) δ 8.90 (s, 1H), 8.75 (s, 1H), 8.46 (d, J = 5.1 Hz, 1H), 7.88 (s, 1H), 7.74 (s, 1H), 7.68 (d, J = 5.2 Hz, 1H), 7.60 (s, 1H), 3.82-3.78 (m, 1H), 0.62-0.54 (m, 2H), 0.12-0.08 (m, 2H). | 387 |
| | cyclopropoxypyridin-4- | | | |
| | yl)-4H-1,2,4-triazol-3- | | | |
| | yl)-5-(trifluoromethyl)- | | | |
| | 1H-benzo[d]imidazole | | | |
| Example 229 Method 9 | 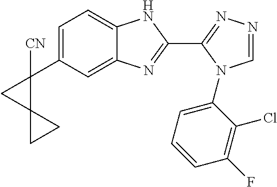 [SMILES: N#CC1(c2ccc3[nH]c(-c4nncn4-c4cccc(F)c4Cl)nc3c2)CC12CC2] | white solid, yield: 25% | ¹H NMR (400 MHz, Methanol-d₄) δ 8.86 (s, 1H), 7.66-7.45 (m, 5H), 7.35-7.09 (m, 1H), 2.20 (m, 1H), 1.93 (m, 1H), 1.37-1.14 (m, 3H), 1.02 (m, 1H). | 405 |
| | 1-(2-(4-(2-chloro-3- | | | |
| | fluorophenyl)-4H-1,2,4- | | | |
| | triazol-3-yl)-1H- | | | |
| | benzo[d]imidazol-5- | | | |
| | yl)spiro[2.2]pentane-1- | | | |
| | carbonitrile | | | |
| Example 230 Method 13 | 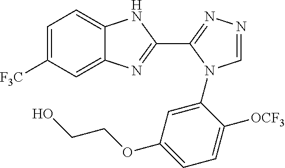 [SMILES: OCCOc1ccc(OC(F)(F)F)c(-n2cnnc2-c2nc3cc(C(F)(F)F)ccc3[nH]2)c1] | White solid, yield: 17.9% | ¹H NMR (400 MHz, DMSO-d₆) δ 14.02 (s, 1H), 9.09 (s, 1H), 7.84 (d, J = 7.0 Hz, 1H), 7.72 (d, J = 8.6 Hz, 1H), 7.62-7.48 (m, 3H), 7.27 (dt, J = 9.2, 4.7 Hz, 1H), 4.09 (t, J = 4.8 Hz, 2H), 3.74 (t, J = 4.8 Hz, 2H). | 473 |
| | 2-(4- | | | |
| | (trifluoromethoxy)-3- | | | |
| | (3-(5-(trifluoromethyl)- | | | |
| | 1H-benzo[d]imidazol-2- | | | |
| | yl)-4H-1,2,4-triazol-4- | | | |
| | yl)phenoxy)ethan-1-ol | | | |
| Example 231 Method 13 | 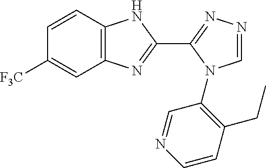 [SMILES: CCc1ccncc1-n1cnnc1-c1nc2cc(C(F)(F)F)ccc2[nH]1] | white solid, yield: 7% | ¹H NMR (400 MHz, DMSO-d₆) δ 9.08 (s, 1H), 8.70 (d, J = 4.8 Hz, 1H), 8.63 (s, 1H), 7.80 (s, 1H), 7.68 (d, J = 8.4 Hz, 1H), 7.57 (d, J = 4.8 Hz, 1H), 7.52 (d, J = 8.4 Hz, 1H), 2.34 (q, J = 7.6 Hz, 2H), 0.99 (t, J = 7.6 Hz, 3H). | 359 |
| | 2-(4-(4-ethylpyridin-3- | | | |
| | yl)-4H-1,2,4-triazol-3- | | | |
| | yl)-5-(trifluoromethyl)- | | | |
| | 1H-benzo[d]imidazole | | | |
| Example 232 Method 13 | 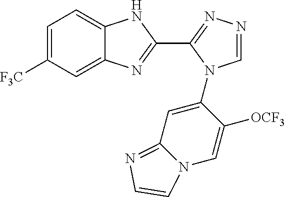 [SMILES: FC(F)(F)Oc1cn2ccnc2cc1-n1cnnc1-c1nc2cc(C(F)(F)F)ccc2[nH]1] | Off- white solid, yield: 73.0% | ¹H NMR (400 MHz, DMSO-d₆) δ 14.05 (s, 1H), 9.23 (s, 1H), 9.13 (s, 1H), 8.32 (s, 1H), 8.21 (s, 1H), 7.86 (d, J = 4.5 Hz, 2H), 7.71 (d, J = 8.5 Hz, 1H), 7.52 (d, J = 8.6 Hz, 1H). | 454 |
| | 2-(4-(6- | | | |
| | (trifluoromethoxy)imidazo | | | |
| | [1,2-a]pyridin-7-yl)- | | | |
| | 4H-1,2,4-triazol-3-yl)- | | | |
| | 5-(trifluoromethyl)-1H- | | | |
| | benzo[d]imidazole | | | |
| Example 233 Method 13 | 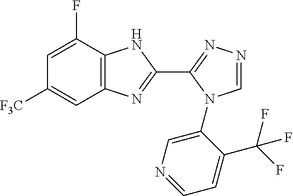 [SMILES: Fc1cc(C(F)(F)F)cc2nc(-c3nncn3-c3cnccc3C(F)(F)F)[nH]c12] | white solid, | ¹H NMR (400 MHz, DMSO-d₆) δ 14.56 (b, 1H), 9.21 (s, 1H), 9.18- 9.07 (m, 2H), 8.10 (d, J = 5.1 Hz, 1H), 7.66 (s, 1H), 7.42 (d, J = 10.8 Hz, 1H). | 417 |
| | 7-Fluoro-5- | | | |
| | (trifluoromethyl)-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazole | | | |
| Example 234 Method 13 | 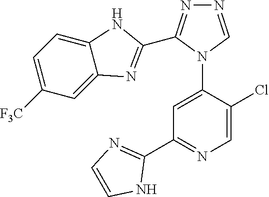 [SMILES: FC(F)(F)c1ccc2[nH]c(-c3nncn3-c3cc(-c4ncc[nH]4)ncc3Cl)nc2c1] | White solid, yield: 13.5% | ¹HNMR (400 MHz, MeOD) δ 9.01 (s, 1H), 8.77 (s, 1H), 8.61 (s, 1H), 8.30 (s, 1H), 7.96 (s, 1H), 7.73-7.83 (m, 1H), 7.59-7.61 (m, 1H), 7.19 (s, 1H). | 431 |
| | 2-(4-(5-chloro-2-(1H- | | | |
| | imidazol-2-yl)pyridin- | | | |
| | 4-yl)-4H-1,2,4-triazol- | | | |
| | 3-yl)-5- | | | |
| | (trifluoromethyl)-1H- | | | |
| | benzo[d]imidazole | | | |
| Example 235 Method 13 | 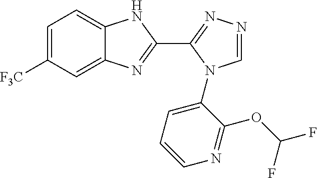 [SMILES: FC(F)Oc1ncccc1-n1cnnc1-c1nc2cc(C(F)(F)F)ccc2[nH]1] | White solid, yield: 33% | ¹H NMR (400 MHz, DMSO-d₆) δ 14.07 (s, 1H), 9.10 (s, 1H), 8.49 (dd, J = 5.0 Hz, J = 1.7 Hz, 1H), 8.30 (dd, J = 7.7 Hz, J =1.7 Hz, 1H), 7.85 (s, 1H), 7.71 (d, J = 8.4 Hz, 1H), 7.59 (t, J = 45.2 Hz, 1H), 7.58- 7.53 (m, 2H). | 397 |
| | 2-(4-(2- | | | |
| | (difluoromethoxy)pyridin- | | | |
| | 3-yl)-4H-1,2,4- | | | |
| | triazol-3-yl)-5- | | | |
| | (trifluoromethyl)-1H- | | | |
| | benzo[d]imidazole | | | |
| Example 236 Method 13 | 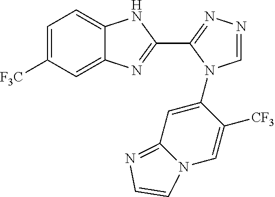 [SMILES: FC(F)(F)c1ccc2[nH]c(-c3nncn3-c3cc4nccn4cc3C(F)(F)F)nc2c1] | White solid, yield: 22.1% | ¹HNMR (400 MHz, DMSO-d₆) δ 14.00 (s, 1H), 9.51 (s, 1H), 9.11 (s, 1H), 8.30 (d, J = 6.8 Hz, 2H), 7.93 (d, J = 1.2 Hz, 1H), 7.80 (s, 1H), 7.68 (d, J = 8.4 Hz, 1H), 7.51 (s, 1H). | 438 |
| | 5-(trifluoromethyl)-2- | | | |
| | (4-(6- | | | |
| | (trifluoromethyl)imidazo | | | |
| | [1,2-a]pyridin-7-yl)- | | | |
| | 4H-1,2,4-triazol-3-yl)- | | | |
| | 1H-benzo[d]imidazole | | | |
| Example 237 Method 13 | 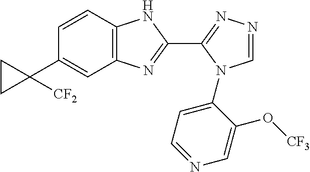 | white solid, yield: 39% | ¹H NMR (400 MHz, Methanol-d₄) δ 8.97 (s, 1H), 8.88-8.76 (m, 2H), 7.88 (d, J = 5.1 Hz, 1H), 7.68-7.27 (m, 3H), 5.68 (td, J = 57.0, 17.8 Hz, 1H), 1.21- 1.09 (m, 2H), 1.05-0.94 (m, 2H). | 437 |
| | (difluoromethyl)cyclopropyl)- | | | |
| | 2-(4-(3- | | | |
| | (trifluoromethoxy)pyridin- | | | |
| | 4-yl)-4H-1,2,4- | | | |
| | triazol-3-yl)-1H- | | | |
| | benzo[d]imidazole | | | |
| Example 238 Method 13 | 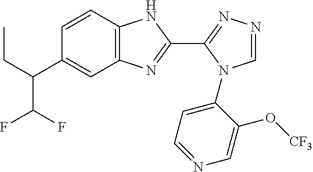 [SMILES: CCC(c1ccc2[nH]c(-c3nncn3-c3ccncc3OC(F)(F)F)nc2c1)C(F)F] | white solid, yield: 17% | ¹H NMR (400 MHz, Methanol-d₄) δ 8.97 (s, 1H), 8.87-8.79 (m, 2H), 7.88 (d, J = 5.2 Hz, 1H), 7.57-7.38 (m, 2H), 7.26-7.14 (m, 1H), 6.11-5.79 (m, 2H), 2.03-1.93 (m, 2H), 1.88-1.75 (m, 1H), 0.82 (t, J = 7.3 Hz, 3H). | 439 |
| | 5-(1,1-difluorobutan-2- | | | |
| | yl)-2-(4-(3- | | | |
| | (trifluoromethoxy)pyridin- | | | |
| | 4-yl)-4H-1,2,4- | | | |
| | triazol-3-yl)-1H- | | | |
| | benzo[d]imidazole | | | |
| Example 239 Method 13 mixture of stereoisomers | 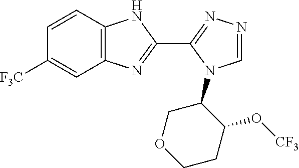 [SMILES: FC(F)(F)O[C@@H]1CCOC[C@H]1n1cnnc1-c1nc2cc(C(F)(F)F)ccc2[nH]1] | white solid, yield: 29% | ¹H NMR (400 MHz, Methanol-d₄) δ 9.10 (s, 1H), 8.05 (d, J = 66.4 Hz, 1H), 7.72 (d, J = 58.4 Hz, 1H), 7.30- 7.06 (m, 1H), 5.93 (m, 1H), 5.23 (m, 1H), 4.32 (dd, J = 11.6, 4.8 Hz, 1H), 4.17-4.08 (m, 1H), 3.98 (t, J = 10.4 Hz, | 422 |
| | | | 1H), 3.83-3.73 (m, | |
| | 2-(4-((3R,4R)-4- | | 1H), 2.32 (d, J = 15.8 | |
| | (trifluoromethoxy) | | Hz, 1H), 2.03 (m, 1H). | |
| | tetrahydro-2H-pyran-3-yl)- | | | |
| | 4H-1,2,4-triazol-3-yl)- | | | |
| | 5-(trifluoromethyl)-1H- | | | |
| | benzo[d]imidazole | | | |
| Example 240 Method 13 | 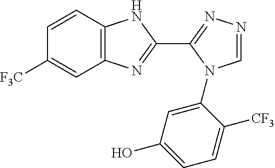 [SMILES: Oc1ccc(C(F)(F)F)c(-n2cnnc2-c2nc3cc(C(F)(F)F)ccc3[nH]2)c1] | white solid, yield: 24% | ¹H NMR (400 MHz, DMSO-d₆) δ 13.98 (d, J = 8.8 Hz, 1H), 10.92 (d, J = 1.6 Hz, 1H), 9.05 (s, 1H), 8.01-7.65 (m, 3H), 7.52 (dd, J = 53.8, 8.2 Hz, 1H), 7.24-6.94 (m, 2H). | 414 |
| | 4-(trifluoromethyl)-3- | | | |
| | (3-(5-(trifluoromethyl)- | | | |
| | 1H-benzo[d]imidazol-2- | | | |
| | yl)-4H-1,2,4-triazol-4- | | | |
| | yl)phenol | | | |
| Example 241 Method 13 | 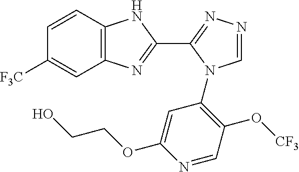 [SMILES: OCCOc1cc(-n2cnnc2-c2nc3cc(C(F)(F)F)ccc3[nH]2)c(OC(F)(F)F)cn1] | white solid, yield: 30% | ¹H NMR (400 MHz, DMSO-d₆) δ 14.14 (d, J = 13.6 Hz, 1H), 9.17 (s, 1H), 8.51 (s, 1H), 7.92- 7.84 (m, 1H), 7.75 (d, J = 8.8 Hz, 1H), 7.63- 7.54 (m, 2H), 4.93 (t, J = 5.6 Hz, 1H), 4.41 (t, J = 4.8 Hz, 2H), 3.79-3.75 (m, 2H). | 475 |
| | 2-((5- | | | |
| | (trifluoromethoxy)-4- | | | |
| | (3-(5-(trifluoromethyl)- | | | |
| | 1H-benzo[d]imidazol-2- | | | |
| | yl)-4H-1,2,4-triazol-4- | | | |
| | yl)pyridin-2- | | | |
| | yl)oxy)ethan-1-ol | | | |
| Example 242 Method 13 | 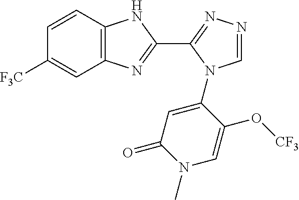 [SMILES: Cn1cc(OC(F)(F)F)c(-n2cnnc2-c2nc3cc(C(F)(F)F)ccc3[nH]2)cc1=O] | yellow solid, yield: 66% | ¹H NMR (400 MHz, DMSO-d₆) δ 14.17 (s, 1H), 9.15 (s, 1H), 8.83 (s, 1H), 8.03 (s, 1H), 7.96 (s, 1H), 7.78 (d, J = 8.4 Hz, 1H), 7.59 (d, J = 8.4 Hz, 1H), 3.95 (s, 3H). | 461 |
| | 1-methyl-5- | | | |
| | (trifluoromethoxy)-4- | | | |
| | (3-(5-(trifluoromethyl)- | | | |
| | 1H-benzo[d]imidazol-2- | | | |
| | yl)-4H-1,2,4-triazol-4- | | | |
| | yl)pyridine-2(1H)- | | | |
| | thione | | | |
| Example 243 Method 9 | 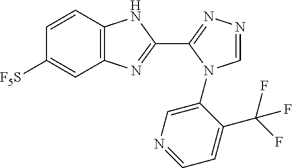 | white solid, yield: 39% | ¹H NMR (400 MHz, DMSO-d₆) δ 14.23 (s, 1H), 9.20 (s, 1H), 9.16- 9.10 (m, 2H), 8.11 (d, J = 5.2 Hz, 1H), 7.99- 7.88 (m, 1H), 7.83-7.55 (m, 2H). | 457 |
| | 4,6-dichloro-2-(4-(3- | | | |
| | (trifluoromethoxy)pyridin- | | | |
| | 4-yl)-4H-1,2,4- | | | |
| | triazol-3-yl)-1H- | | | |
| | benzo[d]imidazole | | | |
| Example 244 Method 13 | 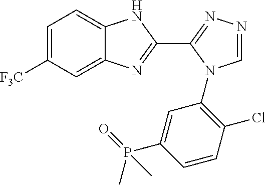 [SMILES: CP(C)(=O)c1ccc(Cl)c(-n2cnnc2-c2nc3cc(C(F)(F)F)ccc3[nH]2)c1] | white solid, yield: 19% | ¹H NMR (400 MHz, DMSO-d₆) δ 14.06 (bs, 1H), 9.14 (s, 1H), 8.19 (dd, J = 11.2 Hz, J = 1.6 Hz, 1H), 8.07-7.97 (m, 1H), 8.19 (dd, J = 8.0 Hz, J = 2.2 Hz, 1H), 7.81 (s, 1H), 7.69 (d, J = 8.0 Hz, 1H), 7.62-7.46 (m, 1H), 1.69 (d, J = 13.6 Hz, 6H). | 440 |
| | (4-chloro-3-(3-(5- | | | |
| | (trifluoromethyl)-1H- | | | |
| | benzo[d]imidazol-2-yl)- | | | |
| | 4H-1,2,4-triazol-4- | | | |
| | yl)phenyl)dimethylphosphine | | | |
| | oxide | | | |
| Example 245 Method 13 | 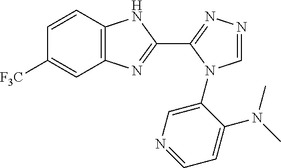 [SMILES: CN(C)c1ccncc1-n1cnnc1-c1nc2cc(C(F)(F)F)ccc2[nH]1] | yellow solid, yield: 16% | ¹H NMR (400 MHz, DMSO-d₆) δ 9.05 (s, 1H), 8.29 (d, J = 6.0 Hz, 1H), 8.27 (s, 1H), 8.19 (s, 1H), 7.87 (s, 1H), 7.74 (d, J = 8.5 Hz, 1H), 7.55 (d, J = 8.5 Hz, 1H), 6.95 (d, J = 6.0 Hz, 1H), 2.59 (s, 6H). | 374 |
| | N,N-dimethyl-3-(3-(5- | | | |
| | (trifluoromethyl)-1H- | | | |
| | benzo[d]imidazol-2-yl)- | | | |
| | 4H-1,2,4-triazol-4- | | | |
| | yl)pyridin-4-amine | | | |
| Example 246 Method 13 | 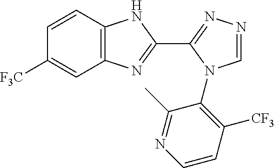 [SMILES: Cc1nccc(C(F)(F)F)c1-n1cnnc1-c1nc2cc(C(F)(F)F)ccc2[nH]1] | white solid, yield: 64 % | ¹H NMR (400 MHz, DMSO-d₆) δ 14.19 (m, 1H), 9.17 (d, J = 2.4 Hz, 1H), 9.01 (d, J = 4.8 Hz, 1H), 7.93 (d, J = 5.2 Hz, 1H), 7.82-7.46 (m, 3H), 2.25 (s, 3H). | 413 |
| | 2-(4-(2-methyl-4- | | | |
| | (trifluoromethyl)pyridin- | | | |
| | 3-yl)-4H-1,2,4-triazol- | | | |
| | 3-yl)-5- | | | |
| | (trifluoromethyl)-1H- | | | |
| | benzo[d]imidazole | | | |
| Example 247 Method 1 | 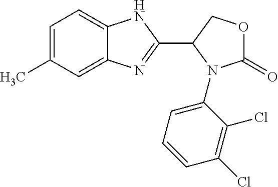 [SMILES: Cc1ccc2[nH]c(C3COC(=O)N3c3cccc(Cl)c3Cl)nc2c1] | white solid, yield: 4% | ¹H NMR (400 MHz, DMSO-d₆) δ 13.15 (s, 1H), 7.94 (s, 1H), 7.74 (d, J = 8.5 Hz, 1H), 7.63 (dd, J = 7.9, 1.8 Hz, 1H), 7.51 (dd, J = 8.5, 1.7 Hz, 1H), 7.38-7.22 (m, 2H), 5.72 (dd, J = 9.0, 6.1 Hz, 1H), 5.02 (t, J = 9.0 Hz, 1H), 4.83 (dd, J = 8.9, 6.1 Hz, 1H). | 416/418 |
| | 3-(2,3-dichlorophenyl)- | | | |
| | 4-(5- | | | |
| | (trifluoromethoxy)-1H- | | | |
| | benzo[d]imidazol-2- | | | |
| | yl)oxazolidin-2-one | | | |
| Example 248 Method 13 | 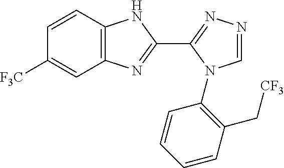 [SMILES: FC(F)(F)Cc1ccccc1-n1cnnc1-c1nc2cc(C(F)(F)F)ccc2[nH]1] | white solid, yield: 14% | 1H NMR (400 MHz, DMSO-d₆) & 13.84 (s, 0.4H), 8.95 (s, 1H), 7.75 (s, 1H), 7.67-7.61 (m, 3H), 7.57-7.51 (m, 2H), 7.48 (d, J = 8.5 Hz, 1H), 3.66-3.51 (m, 2H). | 412 |
| | 2-(4-(2-(2,2,2- | | | |
| | trifluoroethyl)phenyl)- | | | |
| | 4H-1,2,4-triazol-3-yl)- | | | |
| | 5-(trifluoromethyl)-1H- | | | |
| | benzo[d]imidazole | | | |
| Example 249 Method 13 | 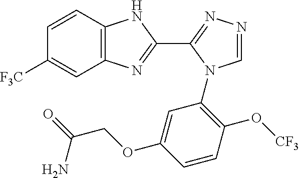 [SMILES: NC(=O)COc1ccc(OC(F)(F)F)c(-n2cnnc2-c2nc3cc(C(F)(F)F)ccc3[nH]2)c1] | White solid, yield: 5% | ¹H NMR (400 MHz, DMSO-d₆) δ 14.02 (s, 1H), 9.09 (s, 1H), 7.85 (s, 1H), 7.72 (d, J = 8.5 Hz, 1H), 7.58 (m, 4H), 7.45 (s, 1H), 7.28 (dd, J = 9.2, 3.2 Hz, 1H), 4.55 (s, 2H). | 487 |
| | 2-(4- | | | |
| | (trifluoromethoxy)-3- | | | |
| | (3-(5-(trifluoromethyl)- | | | |
| | 1H-benzo[d]imidazol-2- | | | |
| | yl)-4H-1,2,4-triazol-4- | | | |
| | yl)phenoxy)acetamide | | | |
| Example 250 Method 13 | 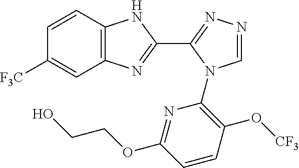 [SMILES: OCCOc1ccc(OC(F)(F)F)c(-n2cnnc2-c2nc3cc(C(F)(F)F)ccc3[nH]2)n1] | white solid, yield: 26% | ¹H NMR (400 MHz, DMSO-d₆) δ 14.16 (bs, 1H), 9.28 (s, 1H), 8.14 (dd, J = 8.9 Hz, J = 1.0 Hz, 1H), 7.87 (s, 1H), 7.74 (d, J = 8.4 Hz, 1H), 7.67-7.44 (m, 1H), 7.27 (d, J = 8.9 Hz, 1H), 4.86 (t, J = 5.5 Hz, 1H), 4.23 (t, J = 4.0 Hz, 2H), 3.67 (dd, J = 10.0, 5.3 Hz, 2H). | 475 |
| | (trifluoromethoxy)-6- | | | |
| | (3-(5-(trifluoromethyl)- | | | |
| | 1H-benzo[d]imidazol-2- | | | |
| | yl)-4H-1,2,4-triazol-4- | | | |
| | yl)pyridin-2- | | | |
| | yl)oxy)ethan-1-ol | | | |
| Example 251 Method 13 | 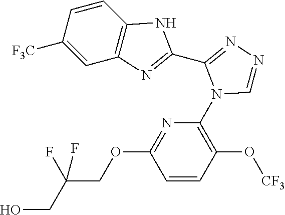 [SMILES: OCC(F)(F)COc1ccc(OC(F)(F)F)c(-n2cnnc2-c2nc3cc(C(F)(F)F)ccc3[nH]2)n1] | white solid, yield: 38% | ¹H NMR (400 MHz, DMSO-d₆) δ 14.17 (bs, 1H), 9.29 (s, 1H), 8.22 (d, J = 8.0 Hz, 1H), 7.86 (s, 1H), 7.78-7.68 (m, 1H), 7.66-7.46 (m, 1H), 7.41 (d, J = 8.8 Hz, 1H), 5.66 (s, 1H), 4.56 (t, J = 13.6 Hz, 2H), 3.73 (t, J = 13.6 Hz, 2H). | 525 |
| | 2,2-difluoro-3-((5- | | | |
| | (trifluoromethoxy)-6- | | | |
| | (3-(5-(trifluoromethyl)- | | | |
| | 1H-benzo[d]imidazol-2- | | | |
| | yl)-4H-1,2,4-triazol-4- | | | |
| | yl)pyridin-2- | | | |
| | yl)oxy)propan-1-ol | | | |
| Example 252 Method 13 | 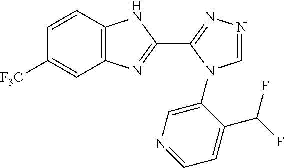 [SMILES: FC(F)c1ccncc1-n1cnnc1-c1nc2cc(C(F)(F)F)ccc2[nH]1] | white solid, yield: 43% | ¹H NMR (400 MHz, DMSO-d₆) δ 14.03 (s, 1H), 9.09 (s, 1H), 9.00 (d, J = 4.8 Hz, 1H), 8.94 (s, 1H), 7.88 (d, J = 4.8 Hz, 1H), 7.78 (s, 1H), 7.67 (d, J = 8.4 Hz, 1H), 7.53 (d, J = 8.4 Hz, 1H), 7.08 (t, J = 53.3 Hz, 1H). | 381 |
| | 2-(4-(4- | | | |
| | (difluoromethyl)pyridin- | | | |
| | 3-yl)-4H-1,2,4-triazol- | | | |
| | 3-yl)-5- | | | |
| | (trifluoromethyl)-1H- | | | |
| | benzo[d]imidazole | | | |
| Example 253 Method 13 | 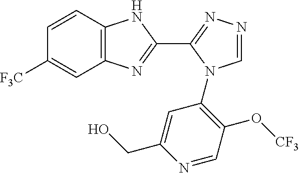 [SMILES: OCc1cc(-n2cnnc2-c2nc3cc(C(F)(F)F)ccc3[nH]2)c(OC(F)(F)F)cn1] | white solid, yield: 31% | ¹H NMR (400 MHz, DMSO-d₆) δ 14.13 (s, 1H), 9.20 (s, 1H), 8.86- 8.85 (m, 1H), 7.99 (s, 1H), 7.85 (s, 1H), 7.75- 7.60 (m, 2H), 5.78-5.75 (m, 1H), 4.73-4.72 (m, 2H). | 445 |
| | (5-(trifluoromethoxy)- | | | |
| | 4-(3-(5- | | | |
| | (trifluoromethyl)-1H- | | | |
| | benzo[d]imidazol-2-yl)- | | | |
| | 4H-1,2,4-triazol-4- | | | |
| | yl)pyridin-2- | | | |
| | yl)methanol | | | |
| Example 254 Method 13 | 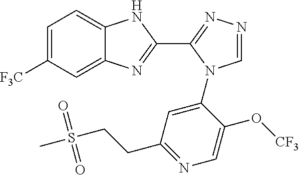 [SMILES: CS(=O)(=O)CCc1cc(-n2cnnc2-c2nc3cc(C(F)(F)F)ccc3[nH]2)c(OC(F)(F)F)cn1] | white solid, yield: 69% | ¹H NMR (400 MHz, DMSO-d₆) δ 14.18 (s, 1H), 9.19 (s, 1H), 8.89 (s, 1H), 8.09 (s, 1H), 7.86 (s, 1H), 7.73 (d, J = 8.4 Hz, 1H), 7.58-7.56 (m, 1H), 3.67-3.63 (m, 2H), 3.38-3.34 (m, 2H), 3.04 (s, 3H). | 521 |
| | 2-(4-(2-(2- | | | |
| | (methylsulfonyl)ethyl)-5- | | | |
| | (trifluoromethoxy)pyridin- | | | |
| | 4-yl)-4H-1,2,4- | | | |
| | triazol-3-yl)-5- | | | |
| | (trifluoromethyl)-1H- | | | |
| | benzo[d]imidazole | | | |
| Example 255 Method 13 | 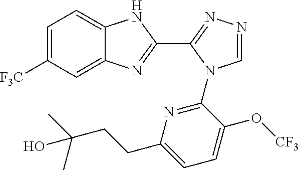 [SMILES: CC(C)(O)CCc1ccc(OC(F)(F)F)c(-n2cnnc2-c2nc3cc(C(F)(F)F)ccc3[nH]2)n1] | white solid, yield: 31% | ¹H NMR (400 MHz, DMSO-d₆) δ 14.14 (bs, 1H), 9.27 (s, 1H), 8.14 (dd, J = 8.8 Hz, J = 1.2 Hz, 1H), 7.86 (s, 1H), 7.73 (d, J = 8.4 Hz, 1H), 7.56 (d, J = 8.4 Hz, 1H), 7.27 (d, J = 8.8 Hz, 1H), 4.65 (s, 1H), 3.93 (s, 2H), 1.12 (s, 6H). | 503 |
| | 2-methyl-1-((5- | | | |
| | (trifluoromethoxy)-6- | | | |
| | (3-(5-(trifluoromethyl)- | | | |
| | 1 H-benzo[d]imidazol-2- | | | |
| | yl)-4H-1,2,4-triazol-4- | | | |
| | yl)pyridin-2- | | | |
| | yl)oxy)propan-2-ol | | | |
| Example 256 Method 13 | 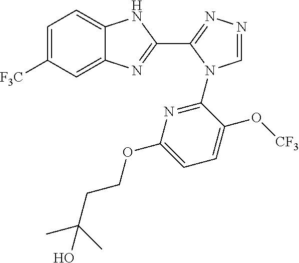 [SMILES: CC(C)(O)CCOc1ccc(OC(F)(F)F)c(-n2cnnc2-c2nc3cc(C(F)(F)F)ccc3[nH]2)n1] | white solid, yield: 33% | ¹H NMR (400 MHz, DMSO-d₆) δ 14.16 (d, J = 14.7 Hz, 1H), 9.29 (s, 1H), 8.14 (dd, J = 8.8 Hz, J = 0.9 Hz, 1H), 7.87 (s, 1H), 7.78-7.68 (m, 1H), 7.64-7.46 (m, 1H), 7.22 (d, J = 8.8 Hz, 1H), 4.36-4.21 (m, 3H), 1.70 (t, J = 7.3 Hz, 2H), 0.96 (s, 6H). | 517 |
| | 2-methyl-4-((5- | | | |
| | (trifluoromethoxy)-6- | | | |
| | (3-(5-(trifluoromethyl)- | | | |
| | 1H-benzo[d]imidazol-2- | | | |
| | yl)-4H-1,2,4-triazol-4- | | | |
| | yl)pyridin-2- | | | |
| | yl)oxy)butan-2-ol | | | |
| Example 257 Method 13 | 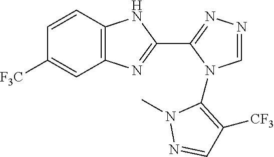 [SMILES: Cn1ncc(C(F)(F)F)c1-n1cnnc1-c1nc2cc(C(F)(F)F)ccc2[nH]1] | white solid, yield: 29.8% | ¹H NMR (400 MHz, DMSO-d₆) δ 9.15 (s, 1H), 8.10 (s, 1H), 7.86 (s, 1H), 7.75 (d, J = 8.5 Hz, 1H), 7.57 (d, J = 8.2 Hz, 1H), 3.73 (s, 3H). | 402 |
| | 2-(4-(1-methyl-4- | | | |
| | (trifluoromethyl)-1H- | | | |
| | pyrazol-5-yl)-4H-1,2,4- | | | |
| | triazol-3-yl)-5- | | | |
| | (trifluoromethyl)-1H- | | | |
| | benzo[d]imidazole | | | |
| Example 258 Method 13 | 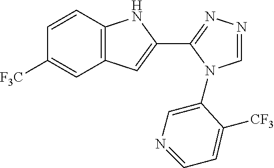 [SMILES: FC(F)(F)c1ccc2[nH]c(-c3nncn3-c3cnccc3C(F)(F)F)cc2c1] | white solid, yield: 66% | ¹H NMR (400 MHz, Methanol-d₄) δ 9.15 (d, J = 5.0 Hz, 1H), 9.00 (s, 1H), 8.85 (s, 1H), 8.07 (d, J = 5.1 Hz, 1H), 7.79- 7.72 (m, 1H), 7.59 (d, J = 8.6 Hz, 1H), 7.44 (dd, J = 8.7, 1.8 Hz, 1H), 5.83 (s, 1H). | 398 |
| | 5-(trifluoromethyl)-2- | | | |
| | (4-(4- | | | |
| | (trifluoromethyl)pyridin- | | | |
| | 3-yl)-4H-1,2,4-triazol- | | | |
| | 3-yl)-1H-indole | | | |
| Example 259 Method 13 | 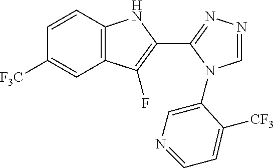 [SMILES: Fc1c(-c2nncn2-c2cnccc2C(F)(F)F)[nH]c2ccc(C(F)(F)F)cc12] | white solid, | ¹H NMR (400 MHz, Methanol-d₄) δ 9.06 (d, J = 5.1 Hz, 1H), 8.95 (s, 1H), 8.91 (s, 1H), 7.99 (d, J = 5.1 Hz, 1H), 7.73 (s, 1H), 7.60-7.54 (m, 1H), 7.54-7.46 (m, 1H). | 416 |
| | 3-fluoro-5- | | | |
| | (trifluoromethyl)-2-(4-(4- | | | |
| | (trifluoromethyl)pyridin- | | | |
| | 3-yl)-4H-1,2,4-triazol- | | | |
| | 3-yl)-1H-indole | | | |
| Example 260 Method 13 | 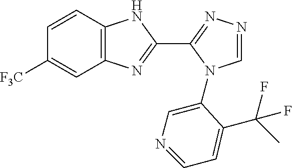 [SMILES: CC(F)(F)c1ccncc1-n1cnnc1-c1nc2cc(C(F)(F)F)ccc2[nH]1] | white solid, | ¹H NMR (400 MHz, DMSO-d₆) δ 14.07 (s, 1H), 9.13 (s, 1H), 8.98 (d, J = 5.2 Hz, 1H), 8.91 (s, 1H), 7.84 (d, J = 5.2 Hz, 1H), 7.78 (s, 1H), 7.67 (d, J = 8.6 Hz, 1H), 7.62-7.43 (m, 1H), 1.78 (t, J = 19.5 Hz, 3H). | 395 |
| | 2-(4-(4-(1,1- | | | |
| | Difluoroethyl)pyridin- | | | |
| | 3-yl)-4H-1,2,4-triazol- | | | |
| | 3-yl)-5- | | | |
| | (trifluoromethyl)-1H- | | | |
| | benzo[d]imidazole | | | |
| Example 261 Method 13 | 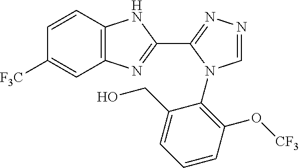 [SMILES: OCc1cccc(OC(F)(F)F)c1-n1cnnc1-c1nc2cc(C(F)(F)F)ccc2[nH]1] | white solid, yield: 28% | ¹H NMR (400 MHz, DMSO-d₆) δ 14.01 (s, 1H), 9.04 (s, 1H), 7.78- 7.67 (m, 4H), 7.55-7.53 (m, 2H), 5.39 (s, 1H), 4.36-4.27 (m, 2H). | 444 |
| | (3-(trifluoromethoxy)- | | | |
| | 2-(3-(5- | | | |
| | (trifluoromethyl)-1H- | | | |
| | benzo[d]imidazol-2-yl)- | | | |
| | 4H-1,2,4-triazol-4- | | | |
| | yl)phenyl)methanol | | | |
| Example 262 Method 13 | 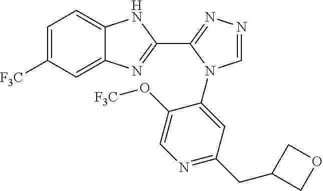 [SMILES: FC(F)(F)Oc1cnc(CC2COC2)cc1-n1cnnc1-c1nc2cc(C(F)(F)F)ccc2[nH]1] | white solid, yield: 5% | ¹H NMR (400 MHz, DMSO-d₆) δ 9.16 (s, 1H), 8.82 (s, 1H), 7.90- 7.85 (m, 2H), 7.73-7.71 (m, 1H), 7.57-7.55 (m, 1H), 4.71-4.68 (m, 2H), 4.43-4.41 (m, 2H), 3.87- 3.69 (m, 2H), 3.27- 3.11 (m, 1H). | 485 |
| | 2-(4-(2-(oxetan-3- | | | |
| | ylmethyl)-5- | | | |
| | (trifluoromethoxy)pyrid | | | |
| | in-4-yl)-4H-1,2,4- | | | |
| | triazol-3-yl)-5- | | | |
| | (trifluoromethyl)-1H- | | | |
| | benzo[d]imidazole | | | |
| Example 263 Method 13 | 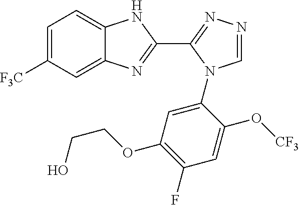 [SMILES: OCCOc1cc(-n2cnnc2-c2nc3cc(C(F)(F)F)ccc3[nH]2)c(OC(F)(F)F)cc1F] | white solid, yield: 24% | ¹H NMR (400 MHz, DMSO-d₆) δ 14.05 (s, 1H), 9.09 (s, 1H), 7.88- 7.56 (m, 4H), 7.48 (s, 1H), 4.96-4.93 (m, 1H), 4.14 (t, J = 4.8 Hz, 2H), 3.76-3.73 (m, 2H). | 492 |
| | 2-(2-fluoro-4- | | | |
| | (trifluoromethoxy)-5- | | | |
| | (3-(5-(trifluoromethyl)- | | | |
| | 1H-benzo[d]imidazol-2- | | | |
| | yl)-4H-1,2,4-triazol-4- | | | |
| | yl)phenoxy)ethan-1-ol | | | |
| Example 264 Method 13 | 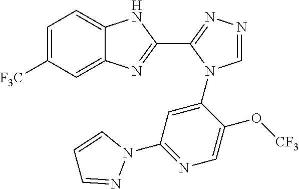 [SMILES: FC(F)(F)Oc1cnc(-n2cccn2)cc1-n1cnnc1-c1nc2cc(C(F)(F)F)ccc2[nH]1] | white solid, yield: 43% | ¹H NMR (400 MHz, DMSO-d₆) δ 14.16 (s, 1H), 9.23 (s, 1H), 8.88 (s, 1H), 8.74-8.73 (m, 1H), 8.52 (s, 1H), 7.93- 7.85 (m, 2H), 7.73-7.53 (m, 2H), 6.70-6.69 (m, 1H). | 481 |
| | 2-(4-(2-(1H-pyrazol-1- | | | |
| | yl)-5- | | | |
| | (trifluoromethoxy)pyridin- | | | |
| | 4-yl)-4H-1,2,4- | | | |
| | triazol-3-yl)-5- | | | |
| | (trifluoromethyl)-1H- | | | |
| | benzo[d]imidazole | | | |
| Example 265 Method 13 | 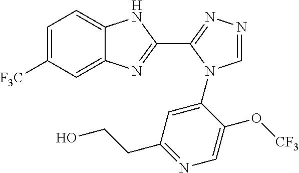 [SMILES: OCCc1cc(-n2cnnc2-c2nc3cc(C(F)(F)F)ccc3[nH]2)c(OC(F)(F)F)cn1] | white solid, yield: 16% | ¹H NMR (400 MHz, DMSO-d₆) δ 14.01 (s, 1H), 9.18 (s, 1H), 8.85- 8.82 (m, 1H), 7.94 (s, 1H), 7.86 (s, 1H), 7.73 (d, J = 8.8 Hz, 1H), 7.59- 7.52 (m, 1H), 4.79- 4.72 (m, 1H), 3.87-3.82 (m, 2H), 3.04 (t, J = 6.8 Hz, 2H). | 459 |
| | (trifluoromethoxy)-4- | | | |
| | (3-(5-(trifluoromethyl)- | | | |
| | 1H-benzo[d]imidazol-2- | | | |
| | yl)-4H-1,2,4-triazol-4- | | | |
| | yl)pyridin-2-yl)ethan-1-o1 | | | |
| Example 266 Method 13 | 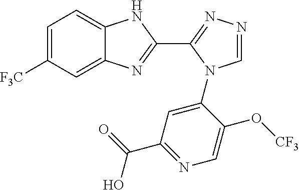 [SMILES: O=C(O)c1cc(-n2cnnc2-c2nc3cc(C(F)(F)F)ccc3[nH]2)c(OC(F)(F)F)cn1] | white solid, yield: 36% | ¹H NMR (400 MHz, DMSO-d₆) δ 9.20 (s, 1H), 8.92 (s, 1H), 8.42 (s, 1H), 7.85 (s, 1H), 7.72 (d, J = 8.4 Hz, 1H), 7.56-7.55 (m, 1H). | 459 |
| | 5-(trifluoromethoxy)-4- | | | |
| | (3-(5-(trifluoromethyl)- | | | |
| | 1H-benzo[d]imidazol-2- | | | |
| | yl)-4H-1,2,4-triazol-4- | | | |
| | yl)picolinic acid | | | |
| Example 267 Method 13 | 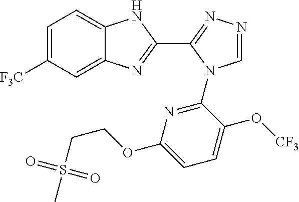 [SMILES: CS(=O)(=O)CCOc1ccc(OC(F)(F)F)c(-n2cnnc2-c2nc3cc(C(F)(F)F)ccc3[nH]2)n1] | white solid, yield: 21% | ¹H NMR (400 MHz, DMSO-d₆) δ 14.18 (bs, 1H), 9.32-9.29 (m 1H), 8.59-8.17 (m, 1H), 7.95- 7.82 (m, 1H), 7.74 (d, J= 8.4 Hz, 1H), 7.67- 7.44 (m, 1H), 7.35 (d, J = 8.8 Hz, 1H), 4.60 (t, J = 5.6 Hz, 2H), 3.61 (t, J = 5.6 Hz, 2H), 3.01 (s, 3H). | 537 |
| | (methylsulfonyl)ethoxy)-3- | | | |
| | (trifluoromethoxy)pyridin- | | | |
| | 2-yl)-4H-1,2,4- | | | |
| | triazol-3-yl)-5- | | | |
| | (trifluoromethyl)-1H- | | | |
| | benzo[d]imidazole | | | |
| Example 268 Method 1 | 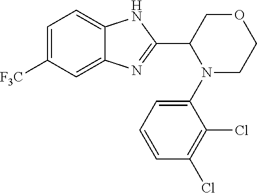 [SMILES: FC(F)(F)c1ccc2[nH]c(C3COCCN3c3cccc(Cl)c3Cl)nc2c1] | White solid, yield: 14% | ¹H NMR (400 MHz, DMSO-d₆) δ 12.07 (s, 1H), 7.61-7.55 (m, 1H), 7.47 (s, 1H), 7.42 (d, J = 7.8 Hz, 1H), 7.31 (dt, J = 25.6, 9.0 Hz, 3H), 5.43 (s, 1H), 4.18 (d, J = 12.2 Hz, 1H), 4.05 (dd, J = 11.8, 4.0 Hz, 2H), 3.90 (d, J = 10.2 Hz, 1H), 3.82- 3.71 (m, 2H). | 546 |
| | 3-(2,3-dichlorophenyl)- | | | |
| | 4-(5-(trifluoromethyl)- | | | |
| | 1H-benzo[d]imidazol-2- | | | |
| | yl)morpholine | | | |
| Example 269 Method 13 | 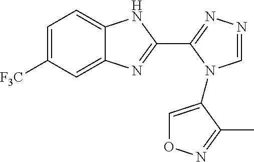 [SMILES: Cc1nocc1-n1cnnc1-c1nc2cc(C(F)(F)F)ccc2[nH]1] | white solid, yield: 27% | ¹H NMR (400 MHz, DMSO-d₆) δ 14.10 (s, 1H), 9.42 (s, 1H), 9.05 (s, 1H), 7.68 (d, J = 67.4 Hz, 3H), 2.15 (s, 3H). | 335 |
| | 3-methyl-4-(3-(5- | | | |
| | (trifluoromethyl)-1H- | | | |
| | benzo[d]imidazol-2-yl)- | | | |
| | 4H-1,2,4-triazol-4- | | | |
| | yl)isoxazole | | | |
| Example 270 Method 13 | 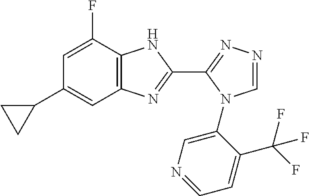 [SMILES: Fc1cc(C2CC2)cc2nc(-c3nncn3-c3cnccc3C(F)(F)F)[nH]c12] | white solid, yield: 60% | ¹H NMR (400 MHz, DMSO-d₆) δ 13.76 (s, 1H), 9.17-9.05 (m, 3H), 8.07 (d, J = 5.2 Hz, 1H), 7.09-7.02 (m, 1H), 6.67 (d, J = 12.3 Hz, 1H), 2.04 (tt, J = 8.7, 5.2 Hz, 1H), 1.02-0.93 (m, 2H), 0.73-0.62 (m, 2H). | 389 |
| | 5-cyclopropyl-7-fluoro- | | | |
| | 2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazole | | | |
| Example 271 Method 13 | 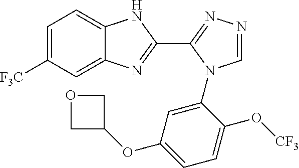 [SMILES: FC(F)(F)Oc1ccc(OC2COC2)cc1-n1cnnc1-c1nc2cc(C(F)(F)F)ccc2[nH]1] | white solid, yield: 15% | ¹H NMR (400 MHz, Methanol-d₄) δ 8.81 (s, 1H), 7.81-7.72 (m, 1H), 7.66-7.57 (m, 1H), 7.54-7.39 (m, 1H), 7.36 (dd, J = 9.2, 1.5 Hz, 1H), 7.15 (d, J = 3.2 Hz, 1H), 7.04 (dd, J = 9.2, 3.2 Hz, 1H), 5.34- 5.23 (m, 1H), 4.91 (t, J = 6.4 Hz, 2H), 4.65-4.61 (m, 2H). | 486 |
| | 2-(4-(5-(oxetan-3- | | | |
| | yloxy)-2- | | | |
| | (trifluoromethoxy)phen | | | |
| | yl)-4H-1,2,4-triazol-3- | | | |
| | yl)-5-(trifluoromethyl)- | | | |
| | 1H-benzo[d]imidazole | | | |
| Example 272 Method 13 | 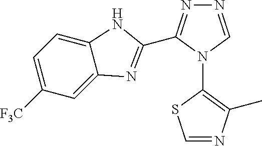 [SMILES: Cc1ncsc1-n1cnnc1-c1nc2cc(C(F)(F)F)ccc2[nH]1] | white solid, yield: 19% | ¹H NMR (400 MHz, DMSO-d₆) δ 14.13- 13.91 (m, 1H), 9.19 (s, 1H), 9.13 (s, 1H), 7.89 (s, 1H), 7.75 (d, J = 8.4 Hz, 1H), 7.57 (d, J = 8.0 Hz, 1H), 2.16 (s, 3H). | 351 |
| | 4-methyl-5-(3-(5- | | | |
| | (trifluoromethyl)-1H- | | | |
| | benzo[d]imidazol-2-yl)- | | | |
| | 4H-1,2,4-triazol-4- | | | |
| | yl)thiazole | | | |
| Example 273 Method 1 | 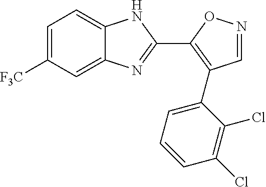 [SMILES: FC(F)(F)c1ccc2[nH]c(-c3oncc3-c3cccc(Cl)c3Cl)nc2c1] | off- white solid, yield: 11% | ¹H NMR (400 MHz, DMSO-d₆) δ 13.96 (s, 1H), 9.13 (s, 1H), 7.83- 7.74 (m, 2H), 7.67-7.58 (m, 2H), 7.55-7.35 (m, 2H). | 398 |
| | 4-(2,3-dichlorophenyl)- | | | |
| | 5-(5-(trifluoromethyl)- | | | |
| | 1H-benzo[d]imidazol-2- | | | |
| | yl)isoxazole | | | |
| Example 274 Method 1 | 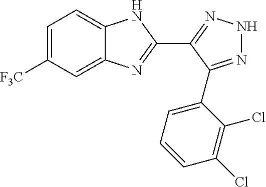 [SMILES: FC(F)(F)c1ccc2[nH]c(-c3n[nH]nc3-c3cccc(Cl)c3Cl)nc2c1] | white solid, yield: 2% | ¹H NMR (400 MHz, Methanol-d₄) δ 8.40 (s, 1H), 7.86 (s, 1H), 7.72- 7.68 (m, 2H), 7.54-7.51 (m, 2H), 7.45 (t, J = 8.0 Hz, 1H). | 398 |
| | 2-(5-(2,3- | | | |
| | dichlorophenyl)-2H- | | | |
| | 1,2,3-triazol-4-yl)-5- | | | |
| | (trifluoromethyl)-1H- | | | |
| | benzo[d]imidazole | | | |
| Example 275 Method 13 | 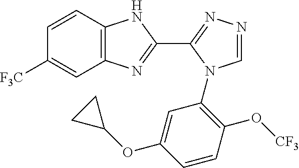 [SMILES: FC(F)(F)Oc1ccc(OC2CC2)cc1-n1cnnc1-c1nc2cc(C(F)(F)F)ccc2[nH]1] | white solid, yield: 37% | ¹H NMR (400 MHz, Methanol-d₄) δ 8.84-8.79 (m, 1H), 7.77 (s, 1H), 7.63 (d, J = 8.8 Hz, 1H), 7.46 (s, 1H), 7.42-7.30 (m, 2H), 7.29-7.23 (m, 1H), 3.83-3.78 (m, 1H), 0.87-0.55 (m, 4H). | 470 |
| | 2-(4-(5-cyclopropoxy-2- | | | |
| | (trifluoromethoxy) | | | |
| | phenyl)-4H-1,2,4-triazol-3- | | | |
| | yl)-5-(trifluoromethyl)- | | | |
| | 1H-benzo[d]imidazole | | | |
| Example 276 Method 9 | 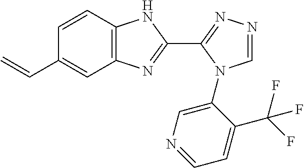 [SMILES: C=Cc1ccc2[nH]c(-c3nncn3-c3cnccc3C(F)(F)F)nc2c1] | white solid, yield: 44% | ¹H NMR (400 MHz, DMSO-d₆) δ 13.67 (bs, 1H), 9.14-9.09 (m, 3H), 8.09 (d, J = 5.2 Hz, 1H), 7.52-7.32 (m, 3H), 6.88-6.72 (m, 1H), 5.78 (d, J = 17.6 Hz, 1H), 5.19 (d, J = 10.8 Hz, 1H). | 357 |
| | 2-(4-(4- | | | |
| | (trifluoromethyl)pyridin | | | |
| | -3-yl)-4H-1,2,4-triazol- | | | |
| | 3-yl)-5-vinyl-1H- | | | |
| | benzo[d]imidazole | | | |
| Example 277 Method 9 | 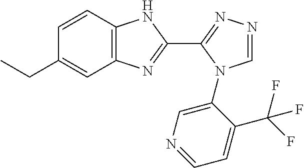 [SMILES: CCc1ccc2[nH]c(-c3nncn3-c3cnccc3C(F)(F)F)nc2c1] | white solid, yield: 25% | ¹H NMR (400 MHz, DMSO-d₆) δ 13.30 (bs, 1H), 9.15-9.02 (m, 3H), 8.08 (d, J = 4.8 Hz, 1H), 7.32 (d, J = 8.0 Hz, 1H), 7.24 (s, 1H), 7.04 (d, J = 8.0 Hz, 1H), 2.65 (q, J = 7.6 Hz, 2H), 1.17 (t, J = 7.6 Hz, 3H). | 359 |
| | 5-ethyl-2-(4-(4- | | | |
| | (trifluoromethyl)pyridin- | | | |
| | 3-yl)-4H-1,2,4-triazol- | | | |
| | 3-yl)-1H- | | | |
| | benzo [d]imidazole | | | |
| Example 278 Method 13 | 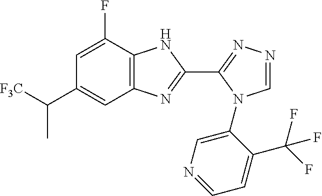 [SMILES: CC(c1cc(F)c2[nH]c(-c3nncn3-c3cnccc3C(F)(F)F)nc2c1)C(F)(F)F] | whitesolid, yield: 75% | ¹H NMR (400 MHz, DMSO-d₆) δ 14.06 (s, 1H), 9.20-9.08 (m, 3H), 8.08 (t, J = 4.3 Hz, 1H), 7.37 (s, 1H), 7.10- 6.95 (m, 1H), 4.06-3.90 (m, 1H), 1.45 (d, J = 7.8, 3.4 Hz, 3H). | 445 |
| | 7-fluoro-2-(4-(4- | | | |
| | (trifluoromethyl)pyridin- | | | |
| | 3-yl)-4H-1,2,4-triazol- | | | |
| | 3-yl)-5-(1,1,1- | | | |
| | trifluoropropan-2-yl)- | | | |
| | 1H-benzo[d]imidazole | | | |
| Example 279 Method 13 | 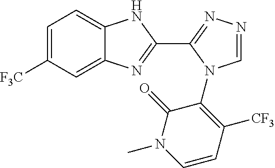 [SMILES: Cn1ccc(C(F)(F)F)c(-n2cnnc2-c2nc3cc(C(F)(F)F)ccc3[nH]2)c1=O] | white solid, yield: 13% | ¹H NMR (400 MHz, DMSO-d₆) δ 8.94 (s, 1H), 8.27 (d, J = 6.8 Hz, 1H), 7.85 (s, 1H), 7.72 (d, J = 8.8 Hz, 1H), 7.56 (d, J = 7.6 Hz, 1H), 6.78 (d, J= 7.2 Hz, 1H), 3.57 (s, 3H). | 429 |
| | 1-methyl-4- | | | |
| | (trifluoromethyl)-3-(3- | | | |
| | (5-(trifluoromethyl)- | | | |
| | 1H-benzo[d]imidazol-2- | | | |
| | yl)-4H-1,2,4-triazol-4- | | | |
| | yl)pyridin-2(1H)-one | | | |
| Example 280 Method 13 | 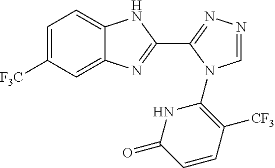 [SMILES: O=c1ccc(C(F)(F)F)c(-n2cnnc2-c2nc3cc(C(F)(F)F)ccc3[nH]2)[nH]1] | white solid, yield: 0.4% | ¹H NMR (400 MHz, DMSO-d₆) δ 14.10 (s, 1H), 12.53 (brs, 1H), 9.25 (s, 1H), 8.27 (d, J = 8.0 Hz, 1H), 7.82 (s, 1H), 7.70 (d, J = 8.4 Hz, 1H), 7.55 (brs, 1H), 7.08 (d, J = 8.4 Hz, 1H). | 415 |
| | 5-(trifluoromethyl)-6- | | | |
| | (3-(5-(trifluoromethyl)- | | | |
| | 1H-benzo[d]imidazol-2- | | | |
| | yl)-4H-1,2,4-triazol-4- | | | |
| | yl)pyridin-2(1H)-one | | | |
| Example 281 Method 13 | 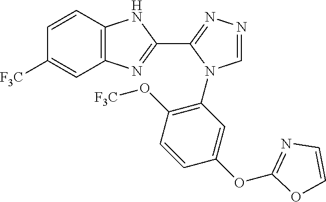 [SMILES: FC(F)(F)Oc1ccc(Oc2ncco2)cc1-n1cnnc1-c1nc2cc(C(F)(F)F)ccc2[nH]1] | white solid, yield: 8% | ¹H NMR (400 MHz, Methanol-d₄) δ 8.87 (s, 1H), 7.87 (d, J = 2.8 Hz, 1H), 7.77 (s, 1H), 7.68 (dd, J = 9.2, 2.8 Hz, 1H), 7.62 (d, J = 8.4 Hz, 1H), 7.57-7.51 (m, 2H), 7.46 (d, J = 8.4 Hz, 1H), 6.88 (s, 1H). | 497 |
| | 2-(4- | | | |
| | (trifluoromethoxy)-3- | | | |
| | (3-(5-(trifluoromethyl)- | | | |
| | 1H-benzo[d]imidazol-2- | | | |
| | yl)-4H-1,2,4-triazol-4- | | | |
| | yl)phenoxy)oxazole | | | |
| Example 282 Method 13 | 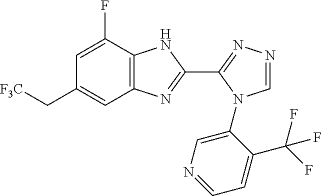 [SMILES: Fc1cc(CC(F)(F)F)cc2nc(-c3nncn3-c3cnccc3C(F)(F)F)[nH]c12] | white solid, yield: 35% | ¹H NMR (400 MHz, DMSO-d₆) δ 14.07 (s, 1H), 9.16 (s, 1H), 9.13- 9.03 (m, 2H), 8.09 (d, J = 5.2 Hz, 1H), 7.38 (s, 1H), 6.99 (d, J = 11.5 Hz, 1H), 3.88-3.63 (m, 2H). | 431 |
| | 7-fluoro-5-(2,2,2- | | | |
| | trifluoroethyl)-2-(4-(4- | | | |
| | (trifluoromethyl)pyridin | | | |
| | -3-yl)-4H-1,2,4-triazol- | | | |
| | 3-yl)-1H- | | | |
| | benzo[d]imidazole | | | |
| Example 283 Method 4 | 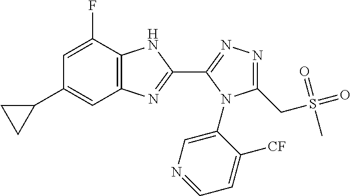 | white solid, yield: 61% | ¹H NMR (400 MHz, DMSO-d₆) δ 13.80 (s, 1H), 9.13 (d, J = 5.1 Hz, 1H), 8.94 (s, 1H), 8.05 (d, J = 5.2 Hz, 1H), 7.04 (s, 1H), 6.67 (d, J = 12.2 Hz, 1H), 5.09 (d, J = 15.0 Hz, 1H), 4.72 (d, J = 15.1 Hz, 1H), 3.19 (s, 3H), 2.09-1.99 (m, 1H), 1.02-0.92 (m, 2H), 0.74-0.63 (m, 2H). | 481 |
| | 5-cyclopropyl-7-fluoro- | | | |
| | 2-(5- | | | |
| | ((methylsulfonyl) | | | |
| | methyl)-4-(4- | | | |
| | (trifluoromethyl)pyridin- | | | |
| | 3-yl)-4H-1,2,4-triazol- | | | |
| | 3-yl)-1H- | | | |
| | benzo[d]imidazole | | | |
| Example 284 Method 13 | 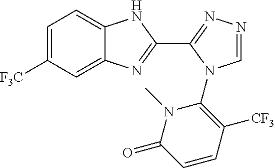 [SMILES: Cn1c(-n2cnnc2-c2nc3cc(C(F)(F)F)ccc3[nH]2)c(C(F)(F)F)ccc1=O] | white solid, yield: 32% | ¹H NMR (400 MHz, DMSO-d₆) δ 14.35 (s, 1H), 9.24 (s, 1H), 7.90-7.87 (m, 2H), 7.75 (d, J = 8.4 Hz, 1H), 7.58 (d, J = 6.8 Hz, 1H), 6.90 (d, J = 10.0 Hz, 1H), 3.12 (s, 3H). | 429 |
| | 1-methyl-5- | | | |
| | (trifluoromethyl)-6-(3- | | | |
| | (5-(trifluoromethyl)- | | | |
| | 1H-benzo[d]imidazol-2- | | | |
| | yl)-4H-1,2,4-triazol-4- | | | |
| | yl)pyridin-2(1H)-one | | | |
| Example 285 Method 9 | 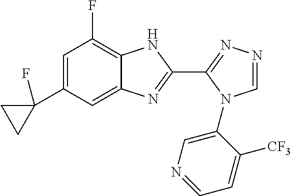 [SMILES: Fc1cc(C2(F)CC2)cc2nc(-c3nncn3-c3cnccc3C(F)(F)F)[nH]c12] | white solid, yield: 17% | ¹H NMR (400 MHz, Methanol-d₄) δ 9.06 (d, J = 5.1 Hz, 1H), 8.95 (d, J = 8.0 Hz, 2H), 7.98 (d, J = 5.1 Hz, 1H), 7.34 (s, 1H), 6.79 (d, J = 11.7 Hz, 1H), 1.56-1.39 (m, 2H), 1.19-1.06 (m, 2H). | 407 |
| | 7-fluoro-5-(1- | | | |
| | fluorocyclopropyl)-2- | | | |
| | (4-(4- | | | |
| | (trifluoromethyl)pyridin- | | | |
| | 3-yl)-4H-1,2,4-triazol- | | | |
| | 3-yl)-1H- | | | |
| | benzo[d]imidazole | | | |
| Example 286 Method 9 | 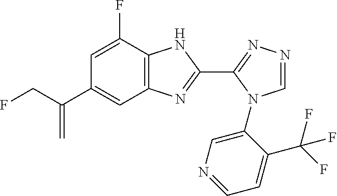 [SMILES: C=C(CF)c1cc(F)c2[nH]c(-c3nncn3-c3cnccc3C(F)(F)F)nc2c1] | white solid, yield: 10% | ¹H NMR (400 MHz, Methanol-d₄) δ 9.07 (d, J = 5.1 Hz, 1H), 8.96 (d, J = 8.3 Hz, 2H), 7.98 (d, J = 5.2 Hz, 1H), 7.45 (s, 1H), 7.12 (s, 1H), 5.66 (s, 1H), 5.46 (s, 1H), 5.31 (s, 1H), 5.19 (s, 1H). | 407 |
| | 7-fluoro-5-(3- | | | |
| | fluoroprop-1-en-2-yl)- | | | |
| | 2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazole | | | |
| Example 287 Method 13 | 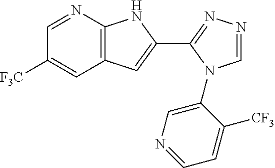 [SMILES: FC(F)(F)c1cnc2[nH]c(-c3nncn3-c3cnccc3C(F)(F)F)cc2c1] | white solid, | ¹H NMR (400 MHz, DMSO-d₆) δ 13.25 (s, 1H), 9.22 (t, J = 2.8 Hz, 2H), 9.06 (s, 1H), 8.62 (d, J = 2.2 Hz, 1H), 8.34 (d, J = 2.3 Hz, 1H), 8.18 (d, J = 5.1 Hz, 1H), 5.83 (d, J = 2.1 Hz, 1H). | 399 |
| | 5-(trifluoromethyl)-2- | | | |
| | (4-(4- | | | |
| | (trifluoromethyl)pyridin- | | | |
| | 3-yl)-4H-1,2,4-triazol- | | | |
| | 3-yl)-1H-pyrrolo[2,3- | | | |
| | b]pyridine | | | |
| Example 288 Method 13 single unknown stereoisomer | 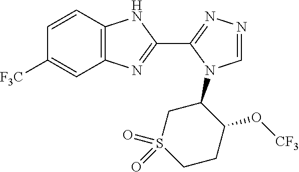 [SMILES: O=S1(=O)CC[C@@H](OC(F)(F)F)[C@H](n2cnnc2-c2nc3cc(C(F)(F)F)ccc3[nH]2)C1] | white solid, yield: 58% | ¹H NMR (400 MHz, DMSO-d₆) δ 14.12 (s, 1H), 9.07 (s, 1H), 8.16-7.84 (m, 2H), 7.65 (d, J = 8.4 Hz, 1H), 6.13 (d, J= 12.8 Hz, 1H), 5.22 (s, 1H), 4.33 (t, J = 13.6 Hz, 1H), 3.94 (d, J = 13.6 Hz, 1H), 3.47- 3.43 (m, 2H), 2.60- | 470 |
| | | | 2.56 (m, 2H). | |
| | Rel-(3S,4R)-4- | | | |
| | (trifluoromethoxy)-3- | | | |
| | (3-(5-(trifluoromethyl)- | | | |
| | 1H-benzo[d]imidazol-2- | | | |
| | yl)-4H-1,2,4-triazol-4- | | | |
| | yl)tetrahydro-2H- | | | |
| | thiopyran 1,1-dioxide | | | |
| Example 289 Method 13 | 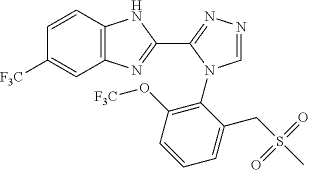 [SMILES: CS(=O)(=O)Cc1cccc(OC(F)(F)F)c1-n1cnnc1-c1nc2cc(C(F)(F)F)ccc2[nH]1] | white solid, yield: 23% | ¹H NMR (400 MHz, DMSO-d₆) δ 14.02 (s, 1H), 8.88 (s, 1H), 7.85-7.67 (m, 6H), 4.65 (d, J = 14.0 Hz, 1H), 4.41 (d, J= 13.6 Hz, 1H), 2.97 (s, 3H). | 506 |
| | 2-(4-(2- | | | |
| | ((methylsulfonyl)methyl)-6- | | | |
| | (trifluoromethoxy) | | | |
| | phenyl)-4H-1,2,4-triazol-3- | | | |
| | yl)-5-(trifluoromethyl)- | | | |
| | 1H-benzo[d]imidazole | | | |
| Example 290 Method 9 | 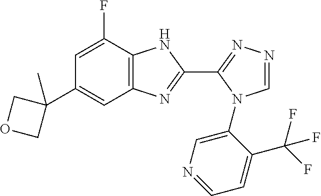 [SMILES: CC1(c2cc(F)c3[nH]c(-c4nncn4-c4cnccc4C(F)(F)F)nc3c2)COC1] | white solid, yield: 32% | ¹H NMR (400 MHz, Methanol-d₄) δ 8.97 (d, J = 5.2 Hz, 1H), 8.86 (d, J = 10.0 Hz, 2H), 7.89 (d, J = 5.2 Hz, 1H), 7.08 (s, 1H), 6.72 (s, 1H), 4.87- 4.85 (m, 2H), 4.57-4.55 (m, 2H), 1.63 (s, 3H). | 419 |
| | 7-fluoro-5-(3- | | | |
| | methyloxetan-3-yl)-2- | | | |
| | (4-(4- | | | |
| | (trifluoromethyl)pyridin- | | | |
| | 3-yl)-4H-1,2,4-triazol- | | | |
| | 3-yl)-1H- | | | |
| | benzo[d]imidazole | | | |
| Example 291 Method 13 | 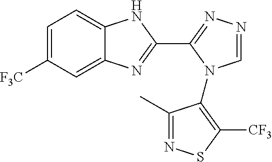 [SMILES: Cc1nsc(C(F)(F)F)c1-n1cnnc1-c1nc2cc(C(F)(F)F)ccc2[nH]1] | white solid, yield: 26% | ¹H NMR (400 MHz, DMSO-d₆) δ 14.22 (s, 1H), 9.20 (s, 1H), 7.84 (d, J = 1.7 Hz, 1H), 7.73 (d, J = 8.5 Hz, 1H), 7.58 (d, J = 8.0 Hz, 1H), 2.25 (s, 3H). | 419 |
| | 5-(difluoromethoxy)-7- | | | |
| | fluoro-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazole | | | |
| Example 292 Method 13 | 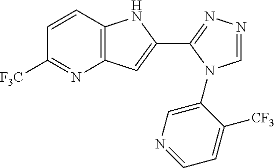 [SMILES: FC(F)(F)c1ccc2[nH]c(-c3nncn3-c3cnccc3C(F)(F)F)cc2n1] | white solid, yield: 15% | ¹H NMR (400 MHz, DMSO-d₆) δ 12.90 (s, 1H), 9.28-9.17 (m, 2H), 9.11 (d, J = 0.8 Hz, 1H), 8.19 (d, J = 5.2 Hz, 1H), 8.03 (d, J = 8.4 Hz, 1H), 7.65 (d, J = 8.4 Hz, 1H), 5.78 (d, J = 0.8 Hz, 1H). | 399 |
| | 5-(trifluoromethyl)-2- | | | |
| | (4-(4- | | | |
| | (trifluoromethyl)pyridin- | | | |
| | 3-yl)-4H-1,2,4-triazol- | | | |
| | 3-yl)-1H-pyrrolo[3,2- | | | |
| | b]pyridine | | | |
| Example 293 Method 9 | 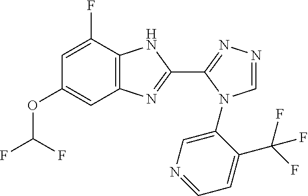 [SMILES: Fc1cc(OC(F)F)cc2nc(-c3nncn3-c3cnccc3C(F)(F)F)[nH]c12] | white solid, yield: 10% | ¹H NMR (400 MHz, DMSO-d₆) δ 14.12 (s, 1H), 9.16-9.10 (m, 3H), 8.09 (d, J = 5.0 Hz, 1H), 7.16 (t, J = 36.9 Hz, 2H), 6.94 (d, J = 11.2 Hz, 1H). | 415 |
| | 5-(difluoromethoxy)-7- | | | |
| | fluoro-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazole | | | |
| Example 294 Method 13 | 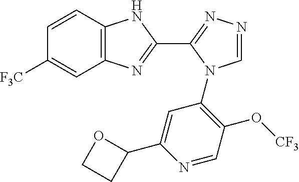 [SMILES: FC(F)(F)Oc1cnc(C2CCO2)cc1-n1cnnc1-c1nc2cc(C(F)(F)F)ccc2[nH]1] | white solid, yield: 4% | ¹H NMR (400 MHz, DMSO-d₆) δ 9.22 (s, 1H), 8.94 (s, 1H), 8.23 (s, 1H), 7.84 (s, 1H), 7.71 (d, J = 8.4 Hz, 1H), 7.55 (s, 1H), 5.85 (t, J = 6.4 Hz, 1H), 4.74-4.61 (m, 2H), 3.20- 3.07 (m, 1H), 2.77-2.72 (m, 1H). | 471 |
| | 2-(4-(2-(oxetan-2-yl)-5- | | | |
| | (trifluoromethoxy)pyridin- | | | |
| | 4-yl)-4H-1,2,4- | | | |
| | triazol-3-yl)-5- | | | |
| | (trifluoromethyl)-1H- | | | |
| | benzo[d]imidazole | | | |
| Example 295 Method 13 | 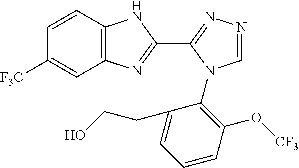 [SMILES: OCCc1cccc(OC(F)(F)F)c1-n1cnnc1-c1nc2cc(C(F)(F)F)ccc2[nH]1] | white solid, yield: 35% | ¹H NMR (400 MHz, DMSO-d₆) δ 14.08 (d, J= 8.8 Hz, 1H), 9.07 (s, 1H), 7.82-7.44 (m, 6H), 4.68 (brs, 1H), 3.55-3.43 (m, 2H), 2.61-2.45 (m, 2H). | 458 |
| | 2-(3- | | | |
| | (trifluoromethoxy)-2- | | | |
| | (3-(5-(trifluoromethyl)- | | | |
| | 1H-benzo[d]imidazol-2- | | | |
| | yl)-4H-1,2,4-triazol-4- | | | |
| | yl)phenyl)ethan-1-ol | | | |
| Example 296 Method 10 | 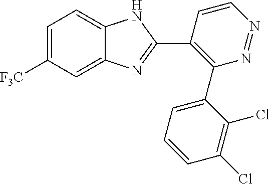 [SMILES: FC(F)(F)c1ccc2[nH]c(-c3ccnnc3-c3cccc(Cl)c3Cl)nc2c1] | white solid, yield: 30% | ¹H NMR (400 MHz, DMSO-d₆) δ 13.51 (s, 1H), 9.60 (d, J = 5.6 Hz, 1H), 8.29 (d, J = 5.2 Hz, 1H), 7.85 (s, 1H), 7.78- 7.72 (m, 2H), 7.58-7.49 (m, 3H). | 409 |
| | 2-(3-(2,3- | | | |
| | dichlorophenyl)pyridazin- | | | |
| | 4-yl)-5- | | | |
| | (trifluoromethyl)-1H- | | | |
| | benzo[d]imidazole | | | |
| Example 297 Method 17 | 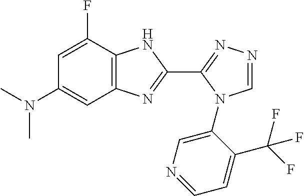 [SMILES: CN(C)c1cc(F)c2[nH]c(-c3nncn3-c3cnccc3C(F)(F)F)nc2c1] | white solid, yield: 22% | ¹H NMR (400 MHz, Methanol-d₄) δ 8.96 (d, J = 5.2 Hz, 1H), 8.85 (s, 1H), 8.81 (s, 1H), 7.88 (d, J = 5.2 Hz, 1H), 6.76- 6.54 (m, 2H), 2.93 (s, 6H). | 392 |
| | 7-fluoro-N,N-dimethyl- | | | |
| | 2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazol-5- | | | |
| | amine | | | |
| Example 298 Method 9 | 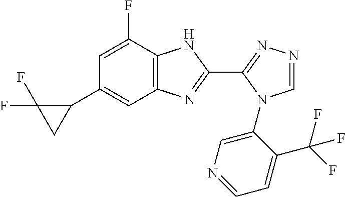 [SMILES: Fc1cc(C2CC2(F)F)cc2nc(-c3nncn3-c3cnccc3C(F)(F)F)[nH]c12] | white solid, yield: 26% | ¹H NMR (400 MHz, DMSO-d₆) δ 13.98 (s, 1H), 9.15 (s, 1H), 9.13- 9.09 (m, 2H), 8.08 (d, J = 5.2 Hz, 1H), 7.24 (s, 1H), 6.92 (m, 1H), 3.14 (m, 1H), 2.05-1.92 (m, 2H). | 425 |
| | 5-(2,2- | | | |
| | difluorocyclopropyl)-7- | | | |
| | fluoro-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazole | | | |
| Example 299 Method 13 | 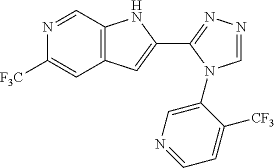 [SMILES: FC(F)(F)c1cc2cc(-c3nncn3-c3cnccc3C(F)(F)F)[nH]c2cn1] | white solid, yield: 42% | ¹H NMR (400 MHz, DMSO-d₆) δ 13.12 (br s, 1H), 9.25-9.19 (m, 2H), 9.09 (s, 1H), 8.88 (s, 1H), 8.19 (d, J = 5.2 Hz, 1H), 8.02 (s, 1H), 5.87 (s, 1H). | 399 |
| | 5-(trifluoromethyl)-2- | | | |
| | (4-(4- | | | |
| | (trifluoromethyl)pyridin- | | | |
| | 3-yl)-4H-1,2,4-triazol- | | | |
| | 3-yl)-1H-pyrrolo[2,3- | | | |
| | c]pyridine | | | |

### 2-(4-(2-chloro-3-fluorophenyl)-4H-1,2,4-triazol-3-yl)-3-(methylsulfonyl)-5-(trifluoromethyl)-1H-indole (346) Method 18

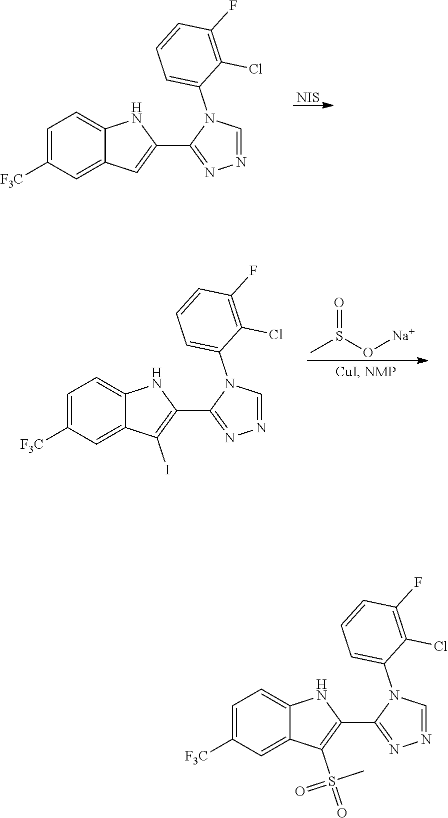

### Step 1. 2-[4-(2-chloro-3-fluorophenyl)-1,2,4-triazol-3-yl]-3-iodo-5-(trifluoromethyl)-1H-indole

A mixture of 2-[4-(2-chloro-3-fluorophenyl)-1,2,4-triazol-3-yl]-5-(trifluoromethyl)-1H-indole (140 mg, 0.36 mmol) in DCM (5 mL) was added NIS (82 mg, 0.36 mmol) in an ice bath. The reaction was stirred at 25° C. for 1 h. The mixture was diluted with water (10 mL), and extracted with DCM (10 mL*2). The combined organic phases were washed with brine (30 mL), dried over Na₂SO₄, filtered and concentrated. The residue was purified by FCC (DCM/MeOH=20/1) to give 2-[4-(2-chloro-3-fluorophenyl)-1,2,4-triazol-3-yl]-3-iodo-5-(trifluoromethyl)-1H-indole (190 mg, 96% yield) MS (ESI) m/z 507 [M+H]⁺.

### Step 2. 2-[4-(2-chloro-3-fluorophenyl)-1,2,4-triazol-3-yl]-3-methanesulfonyl-5-(trifluoromethyl)-1H-indole

To a solution of 2-[4-(2-chloro-3-fluorophenyl)-1,2,4-triazol-3-yl]-3-iodo-5-(trifluoromethyl)-1H-indole (55 mg, 0.10 mmol) in NMP (4 mL) was added Copper(I) iodide (103 mg, 0.54 mmol) and Sodium methanesulfinate (55 mg, 0.54 mmol) at rt. The mixture was stirred at 105° C. for 0.5 h under N2. The mixture was diluted with water (10 mL), and extracted with EA (10 mL*2). The combined organic phases were washed with water (20 mL*2) and brine (30 mL), dried over Na₂SO₄, filtered and concentrated. The residue was purified by prep-HPLC (NH4HCO3) to give (7.0 mg, 11% yield) as a pale yellow solid. MS (ESI) m/z 459 [M+H]⁺. ¹H NMR (400 MHz, DMSO-d₆) δ: 13.38 (s, 0.5H), 9.22 (s, 1H), 8.19 (s, 1H), 7.83-7.35 (m, 5H), 3.06 (s, 3H).

### Preparation of 7-fluoro-5-spiro[2.2]pentan-2-yl-2-[4-[4-(trifluoromethyl)-3-pyridyl]-1,2,4-triazol-3-yl]-1H-benzimidazole (338) Method 19

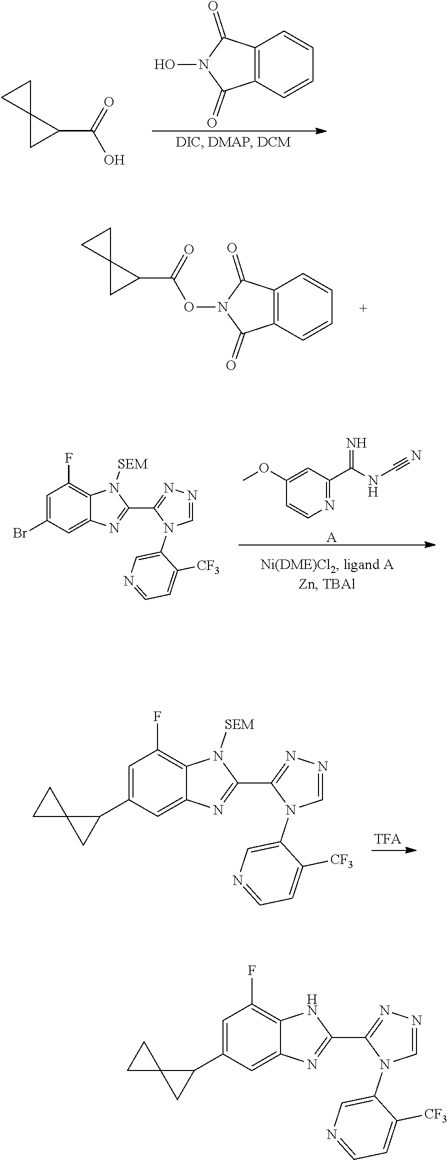

### Step 1. (1,3-dioxoisoindolin-2-yl) spiro[2.2]pentane-2-carboxylate

To a solution of spiro[2.2]pentane-2-carboxylic acid (100 mg, 891.85 μmol, w eq) and 2-hydroxyisoindoline-1,3-dione (160.04 mg, 981.04 μmol, 1.1 eq) in DCM (5 mL) were added DIC (123.81 mg, 981.05 μmol, 151.91 μL, 1.10 eq) and DMAP (10.90 mg, 89.19 μmol, 0.1 eq). The resulting mixture was stirred at 20° C. under N2 protection for 2 hours. LCMS showed no desired mass. TLC indicated spiro[2.2]pentane-2-carboxylic acid (100 tig, 891.85 μmol, 1 eq) was consumed completely and some new spots formed. The reaction mixture was poured into water (5 mL), and extracted with DCM (10 mL×3). The organic layer was washed with brine (5 mL), dried over Na₂SO₄ and filtered. The residue was purified by flash silica gel chromatography (ISCO®; 4 g SepaFlash® Silica Flash Column, Eluent of 0-15% Ethyl acetate/Petroleum ether gradient a @20 mL/min). Compound (1,3-dioxoisoindolin-2-yl) spiro[2.2]pentane-2-carboxylate (160 mg, 621.98 μmol, 69.74% yield) was obtained as a white solid. ¹H NMR (400 MHz, CDCl₃) δ 7.93-7.84 (in, 211), 7.84-7.74 (m, 2H), 2.32 (dd, J=4.2, 7.5 Hz, 111), 1.77 (t, J=4.1 Hz, 1H), 1.72-1.66 (m, 1H), 1.18-1.07 (m, 2H), 1.06-1.00 (in, 2H)

### Step 2. 7-fluoro-5-spiro[2.2]pentan-2-yl-2-[4-[4-(trifluoromethyl)-3-pyridyl]-1,2,4-triazol-3-yl]-1H-benzimidazole

To a solution of 2-[[5-bromo-7-fluoro-2-[4-[4-(trifluoromethyl)-3-pyridyl]-1,2,4-triazol-3-yl]benzimidazol-1-yl]methoxy]ethyl-trimethyl-silane (20 mg, 35.88 μmol, 1 eq) in DMA (1 mL) were added (1,3-dioxoisoindolin-2-yl) spiro[2.2]pentane-2-carboxylate (11.08 mg, 43.06 μmol, 1.2 eq), N-cyano-4-methoxy-pyridine-2-carboxamidine (9.48 mg, 53.82 μmol, 1.5 eq), NiCl₂(DME) (8.67 mg, 39.47 μmol, 1.1 eq) and TBAI (11.93 mg, 32.29 μmol, 0.9 eq) and Zn (7.88 mg, 143.52 μmol, 7.81 μL, 4 eq). The mixture was stirred at 25° C. for 16 hr. LCMS showed desired mass. TLC showed new spots. The reaction mixture was poured into water (2 mL), and extracted with EtOAc (3 mL×3). The organic layers were washed with brine (10 mL), dried over Na₂SO₄ and filtered. The filtrate was concentrated and purified by prep-TLC (Pe:EtOAc=3:1) to give 2-[[7-fluoro-5-spiro[2.2]pentan-2-yl-2-[4-[4-(trifluoromethyl)-3-pyridyl]-1,2,4-triazol-3-yl]benzimidazol-1-yl]methoxy]ethyl-trimethyl-silane (13 mg, 12.41 μmol, 34.59% yield, 52% purity) as a colorless oil. MS (ESI) m/z 545 [M+H]⁺.

### Step 3. 7-fluoro-5-spiro[2.2]pentan-2-yl-2-[4-[4-(trifluoromethyl)-3-pyridyl]-1,2,4-triazol-3-yl]-1H-benzimidazole

To a solution of 2-[[7-fluoro-5-spiro[2.2]pentan-2-yl-2-[4-[4-(trifluoromethyl)-3-pyridyl]-1,2,4-triazol-3-yl]benzimidazol-1-yl]methoxy]ethyl-trimethyl-silane (34 mg, 62.43 μmol, 1 eq) in DCM (1 mL) was added TFA (1.54 g, 13.46 mmol, 1 mL, 215.64 eq). The mixture was stirred at 15° C. for 0.5 hr. LC-MS showed Reactant 1 was consumed completely and one main peak with desired mass was detected. The reaction mixture was concentrated under reduced pressure to give a residue. The residue was purified by prep-HPLC (column: Boston Green ODS 150×30 mm×5 um; mobile phase: [water(TFA)-ACN]; gradient:55%-75% B over 11 min). Compound 7-fluoro-5-spiro[2.2]pentan-2-yl-2-[4-[4-(trifluoromethyl)-3-pyridyl]-1,2,4-triazol-3-yl]-1H-benzimidazole (3.4 mg, 7.83 μmol, 12.54% yield, 95.41% purity) was obtained as a colorless oil. MS (ESI) m/z 415 [M+H]. ¹H NMR (400 MHz, CDCl₃) δ 9.11 (d, J=5.0 Hz, 1H), 8.88 (s, 1H), 8.45 (s, 1H), 7.86 (d, J=5.1 Hz, 1H), 7.27-7.24 (m, 1H), 6.69 (br d, J=11.8 Hz, 1H), 2.33 (dd, J=4.5, 7.8 Hz, 1H), 1.56 (dd, J=4.3, 7.9 Hz, 1H), 1.07-0.91 (m, 4H), 0.75 (br dd, J=4.6, 8.8 Hz, 1H)

| Example No. | | | | |
|---|---|---|---|---|
| (Compound | | Appearance | | |
| No.) | | and | | MS (m/z) |
| Method | Structure and Name | Yield | ¹H NMR Data | [M + H]⁺ |
| Example 300 Method 8 | 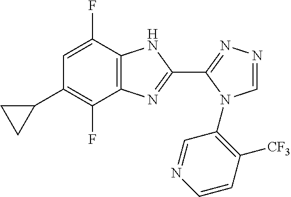 [SMILES: Fc1c(C2CC2)cc(F)c2[nH]c(-c3nncn3-c3cnccc3C(F)(F)F)nc12] | White solid, | ¹H NMR (400 MHz, Methanol-d₄) δ 9.06 (d, J = 5.1 Hz, 1H), 8.96 (d, J = 4.1 Hz, 2H), 7.98 (d, J = 5.1 Hz, 1H), 6.50 (s, 1H), 2.17- 2.03 (m, 1H), 1.08- 0.97 (m, 2H), 0.76- 0.68 (m, 2H). | 407 |
| | 5-cyclopropyl-4,7- | | | |
| | difluoro-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazole | | | |
| Example 301 Method 17 | 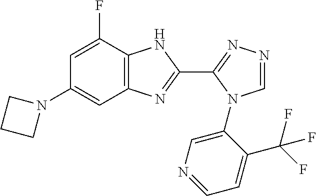 [SMILES: Fc1cc(N2CCC2)cc2nc(-c3nncn3-c3cnccc3C(F)(F)F)[nH]c12] | white solid, yield: 38.9% | ¹H NMR (400 MHz, Methanol-d₄) δ 9.04 (d, J = 5.2 Hz, 1H), 8.93 (s, 1H), 8.88 (s, 1H), 7.96 (d, J = 5.2 Hz, 1H), 6.26 (s, 1H), 6.13 (d, J = 12.4 Hz, 1H), 3.89- 3.83 (m, 4H), 2.36 (p, J = 7.2 Hz, 2H). | 404 |
| | 5-(azetidin-1-yl)-7- | | | |
| | fluoro-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazole | | | |
| Example 302 Method 10 | 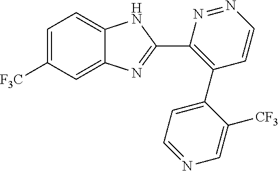 [SMILES: FC(F)(F)c1ccc2[nH]c(-c3nnccc3-c3ccncc3C(F)(F)F)nc2c1] | white solid, | ¹H NMR (400 MHz, DMSO-d₆) δ 14.07 (br, 1H), 9.55 (dd, J = 5.3, 1.8 Hz, 1H), 9.11 (s, 1H), 8.98 (d, J = 5.1 Hz, 1H), 7.95 (dd, J = 5.2, 1.7 Hz, 1H), 7.83- 7.32 (m, 4H). | 410 |
| | 5-(Trifluoromethyl)-2-(4- | | | |
| | (3- | | | |
| | (trifluoromethyl)pyridin- | | | |
| | 4-yl)pyridazin-3-yl)-1H- | | | |
| | benzo[d]imidazole | | | |
| Example 303 Method 10 | 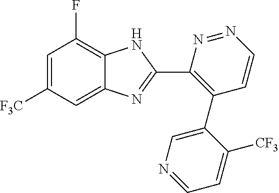 [SMILES: Fc1cc(C(F)(F)F)cc2nc(-c3nnccc3-c3cnccc3C(F)(F)F)[nH]c12] | white solid, | 1H NMR (400 MHz, DMSO-d₆) δ 14.40 (br, 1H), 9.58 (dd, J = 5.2, 1.5 Hz, 1H), 9.00 (d, J = 5.2 Hz, 1H), 8.79 (s, 1H), 8.04 (dd, J = 5.2, 1.5 Hz, 1H), 7.93 (d, J = 5.3 Hz, 1H), 7.69 (s, 1H), 7.35 (d, J = 10.7 Hz, 1H). | 428 |
| | 7-Fluoro-5- | | | |
| | (trifluoromethyl)-2-(4-(4- | | | |
| | (trifluoromethyl)pyridin- | | | |
| | 3-yl)pyridazin-3-yl)-1H- | | | |
| | benzo[d]imidazole | | | |
| Example 304 Method 10 | 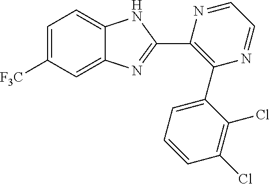 [SMILES: FC(F)(F)c1ccc2[nH]c(-c3nccnc3-c3cccc(Cl)c3Cl)nc2c1] | yellow solid, yield: 10% | ¹H NMR (400 MHz, d₆-DMSO) δ 13.67 (s, 1H), 8.99-8.92 (m, 2H), 7.85-7.40 (m, 6H). | 409 |
| | 2-(3-(2,3- | | | |
| | dichlorophenyl)pyrazin- | | | |
| | 2-yl)-5- | | | |
| | (trifluoromethyl)-1H- | | | |
| | benzo[d]imidazole | | | |
| Example 305 Method 1 | 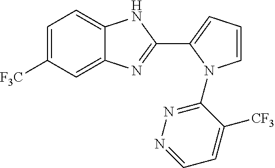 [SMILES: FC(F)(F)c1ccc2[nH]c(-c3cccn3-c3nnccc3C(F)(F)F)nc2c1] | white solid, yield: 16.3% | ¹H NMR (400 MHz, Methanol-d₄) δ 9.48 (d, J = 5.0 Hz, 1H), 8.19 (d, J = 5.0 Hz, 1H), 7.68 (s, 1H), 7.55 (d, J = 8.3 Hz, 1H), 7.51- 7.49 (m, 1H), 7.45 (d, J = 8.1 Hz, 1H), 7.19 (d, J = 3.6 Hz, 1H), 6.64 (t, J = 2.8 Hz, 1H). | 398 |
| | 5-(Trifluoromethyl)-2- | | | |
| | (1-(4- | | | |
| | (trifluoromethyl)pyridazin- | | | |
| | 3-yl)-1H-pyrrol-2- | | | |
| | yl)-1H- | | | |
| | benzo[d]imidazole | | | |
| Example 306 Method 14 | 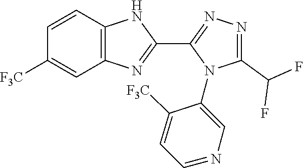 [SMILES: FC(F)c1nnc(-c2nc3cc(C(F)(F)F)ccc3[nH]2)n1-c1cnccc1C(F)(F)F] | white solid, yield: 40.7% | ¹H NMR (400 MHz, DMSO-d₆) δ 14.34 (d, J = 15.6 Hz, 1H), 9.25 (d, J = 3.6 Hz, 1H), 9.19 (d, J = 5.2 Hz, 1H), 8.14 (d, J = 5.2 Hz, 1H), 7.87-7.32 (m, 4H). | 449 |
| | 2-(5-(difluoromethyl)- | | | |
| | 4-(4- | | | |
| | (trifluoromethyl)pyridin- | | | |
| | 3-yl)-4H-1,2,4-triazol- | | | |
| | 3-yl)-5- | | | |
| | (trifluoromethyl)-1H- | | | |
| | benzo[d]imidazole | | | |
| Example 307 Method 4 | 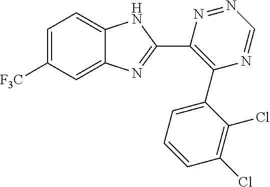 [SMILES: FC(F)(F)c1ccc2[nH]c(-c3nncnc3-c3cccc(Cl)c3Cl)nc2c1] | Off- white solid, yield: 10% | ¹H NMR (400 MHz, DMSO-d₆) δ 11.60 (brs, 1H), 10.08 (s, 1H), 7.85 (dd, J = 8.0, 1.6 Hz, 1H), 7.81 (s, 1H), 7.71 (d, J = 8.4 Hz, 1H), 7.64 (dd, J = 8.0, 2.0 Hz, 1H), 7.58-7.52 (m, 2H). | 410 |
| | 2-(5-(2,3- | | | |
| | dichlorophenyl)-1,2,4- | | | |
| | triazin-6-yl)-5- | | | |
| | (trifluoromethyl)-1H- | | | |
| | benzo[d]imidazole | | | |
| Example 309 Method 13 | 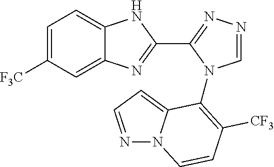 [SMILES: FC(F)(F)c1ccc2[nH]c(-c3nncn3-c3c(C(F)(F)F)ccn4nccc34)nc2c1] | white solid, yield: 16.76% | ¹H NMR (400 MHz, DMSO-d₆) δ 14.15 (s, 1H), 9.28 (s, 1H), 9.24 (d, J = 7.6 Hz, 1H), 8.21 (d, J = 2.4 Hz, 1H), 7.73 (s, 1H), 7.64 (s, 1H), 7.51 (s, 1H), 7.41 (d, J = 7.6 Hz, 1H), 6.58 (d, J = 2.0 Hz, 1H). | 438 |
| | 5-(trifluoromethyl)-2- | | | |
| | (4-(5- | | | |
| | (trifluoromethyl)pyrazolo | | | |
| | [1,5-a]pyridin-4-yl)- | | | |
| | 4H-1,2,4-triazol-3-yl)- | | | |
| | 1H-benzo[d]imidazole | | | |
| Example 310 Method 4 | 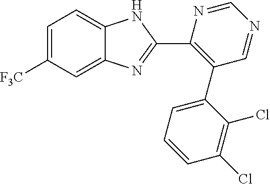 [SMILES: FC(F)(F)c1ccc2[nH]c(-c3ncncc3-c3cccc(Cl)c3Cl)nc2c1] | white solid, yield: 3% | ¹H NMR (400 MHz, DMSO-d₆) δ 13.83 (s, 1H), 9.51 (s, 1H), 8.96 (s, 1H), 7.90-7.60 (m, 3H), 7.60-7.43 (m, 3H). | 409 |
| | 2-(5-(2,3- | | | |
| | dichlorophenyl)pyrimidin- | | | |
| | 4-yl)-5- | | | |
| | (trifluoromethyl)-1H- | | | |
| | benzo[d]imidazole | | | |
| Example 311 Method 13 | 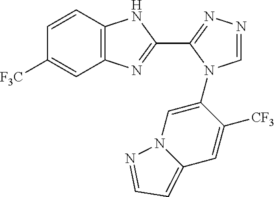 [SMILES: FC(F)(F)c1ccc2[nH]c(-c3nncn3-c3cn4nccc4cc3C(F)(F)F)nc2c1] | white solid, yield: 21.97% | ¹H NMR (400 MHz, DMSO-d₆) δ 14.08 (s, 1H), 9.59 (s, 1H), 9.14 (s, 1H), 8.55 (s, 1H), 8.38 (d, J = 2.4 Hz, 1H), 7.79 (s, 1H), 7.66 (s, 1H), 7.52 (s, 1H), 7.13 (d, J = 2.0 Hz, 1H). | 438 |
| | 5-(trifluoromethyl)-2- | | | |
| | (4-(5- | | | |
| | (trifluoromethyl)pyrazolo | | | |
| | [1,5-a]pyridin-6-yl)- | | | |
| | 4H-1,2,4-triazol-3-yl)- | | | |
| | 1H-benzo[d]imidazole | | | |
| Example 312 Method 13 | 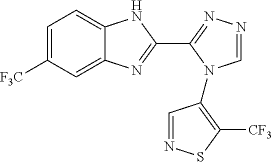 [SMILES: FC(F)(F)c1ccc2[nH]c(-c3nncn3-c3cnsc3C(F)(F)F)nc2c1] | white solid, yield: 26% | ¹H NMR (400 MHz, DMSO-d₆) δ 14.20 (d, J = 12.6 Hz, 1H), 9.21 (s, 1H), 9.12 (s, 1H), 7.84 (s, 1H), 7.72 (dd, J = 16.0, 8.6 Hz, 1H), 7.56 (dd, J = 49.6, 8.4 Hz, 1H). | 405 |
| | 3-(trifluoromethyl)-4- | | | |
| | (3-(5-(trifluoromethyl)- | | | |
| | 1H-benzo[d]imidazol-2- | | | |
| | yl)-4H-1,2,4-triazol-4- | | | |
| | yl)isothiazole | | | |
| Example 313 Method 1 | 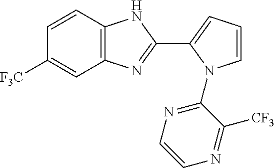 [SMILES: FC(F)(F)c1ccc2[nH]c(-c3cccn3-c3nccnc3C(F)(F)F)nc2c1] | white solid, yield: 26% | ¹H NMR (400 MHz, Methanol-d₄) δ 8.90 (s, 1H), 8.82 (s, 1H), 7.60 (s, 1H), 7.48 (d, J = 8.4 Hz, 1H), 7.40 (d, J = 8.4 Hz, 1H), 7.25 (s, 1H), 7.11 (d, J = 3.7 Hz, 1H), 6.53 (t, J = 2.8 Hz, 1H). | 398 |
| Example 314 Method 8 | 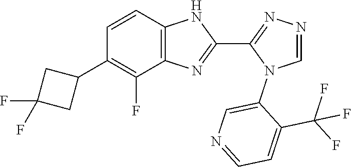 [SMILES: Fc1c(C2CC(F)(F)C2)ccc2[nH]c(-c3nncn3-c3cnccc3C(F)(F)F)nc12] | white solid, yield: 42.8% | ¹H NMR (400 MHz, Methanol-d₄) δ 9.07 (s, 1H), 8.99-8.91 (m, 2H), 7.99 (d, J = 5.1 Hz, 1H), 7.39-7.16 (m, 2H), 3.60 (m, 1H), 3.06-2.89 (m, 2H), 2.85-2.66 (m, 2H). | 439 |
| | 5-(3,3- | | | |
| | difluorocyclobutyl)-4- | | | |
| | fluoro-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazole | | | |
| Example 316 Method 9 | 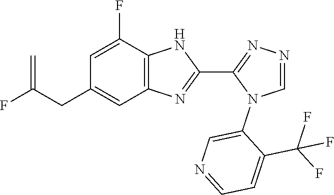 [SMILES: C=C(F)Cc1cc(F)c2[nH]c(-c3nncn3-c3cnccc3C(F)(F)F)nc2c1] | white solid, yield: 13.6% | ¹H NMR (400 MHz, DMSO-d₆) δ 9.17- 9.05 (m, 3H), 8.08 (d, J = 5.2 Hz, 1H), 7.22 (m, 1H), 6.90 (m, 1H), 4.62 (m, 1H), 4.48 (m, 1H), 3.68-3.61 (m, 2H). | 407 |
| | 7-fluoro-5-(2- | | | |
| | fluoroallyl)-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazole | | | |
| Example 317 Method 14 | 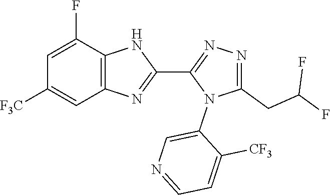 [SMILES: Fc1cc(C(F)(F)F)cc2nc(-c3nnc(CC(F)F)n3-c3cnccc3C(F)(F)F)[nH]c12] | white solid, yield: 11.2% | ¹H NMR (400 MHz, DMSO-d₆) δ 14.55 (s, 1H), 9.17 (d, J = 5.2 Hz, 1H), 9.08 (s, 1H), 8.11 (d, J = 5.2 Hz, 1H), 7.63 (s, 1H), 7.39 (d, J = 10.6 Hz, 1H), 6.51 (tt, J = 55.2, 4.0 Hz, 1H), 3.41 (dd, J = 16.8, 4.4 Hz, 2H). | 481 |
| | 2-(5-(2,2- | | | |
| | difluoroethyl)-4-(4- | | | |
| | (trifluoromethyl)pyridin- | | | |
| | 3-yl)-4H-1,2,4-triazol- | | | |
| | 3-yl)-7-fluoro-5- | | | |
| | (trifluoromethyl)-1H- | | | |
| | benzo[d]imidazole | | | |
| Example 318 Method 17 | 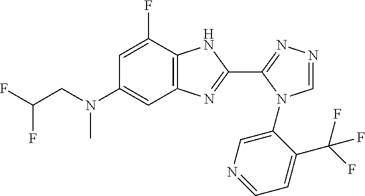 [SMILES: CN(CC(F)F)c1cc(F)c2[nH]c(-c3nncn3-c3cnccc3C(F)(F)F)nc2c1] | white solid, yield: 64.1% | ¹H NMR (400 MHz, DMSO-d₆) δ 13.41 (s, 1H), 9.09 (dd, J = 6.0, 4.0 Hz, 3H), 8.07 (d, J = 5.2 Hz, 1H), 6.65 (dd, J = 14.4, 2.0 Hz, 1H), 6.49 (d, J = 2.0 Hz, 1H), 6.20 (tt, J = 55.2, 3.6 Hz, 1H), 3.78 (td, J = 15.6, 4.0 Hz, 2H), 2.98 (s, 3H). | 442 |
| | N-(2,2-difluoroethyl)-7- | | | |
| | fluoro-N-methyl-2-(4- | | | |
| | (4- | | | |
| | (trifluoromethyl)pyridin- | | | |
| | 3-yl)-4H-1,2,4-triazol- | | | |
| | 3-yl)-1H- | | | |
| | benzo[d]imidazol-5- | | | |
| | amine | | | |
| Example 319 Method 10 | 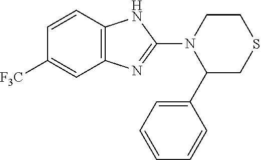 [SMILES: FC(F)(F)c1ccc2[nH]c(N3CCSCC3c3ccccc3)nc2c1] | white solid, yield: 0.67% | ¹H NMR (400 MHz, CDCl₃) δ 7.52 (s, 1H), 7.39-7.32 (m, 7H), 5.44 (s, 1H), 4.52-4.47 (m, 1H), 3.85-3.78 (m, 1H), 3.24-3.13 (m, 2H), 3.08-3.01 (m, 1H), 2.775-3.72 (m, 1H). | 364 |
| | 3-phenyl-4-(5- | | | |
| | (trifluoromethyl)-1H- | | | |
| | benzo[d]imidazol-2- | | | |
| | yl)thiomorpholine | | | |
| Example 320 Method 8 | 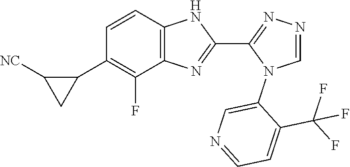 [SMILES: N#CC1CC1c1ccc2[nH]c(-c3nncn3-c3cnccc3C(F)(F)F)nc2c1F] | yellow solid, yield: 52% | ¹H NMR (400 MHz, Methanol-d₄) δ 9.07 (d, J = 5.2 Hz, 1H), 9.00- 8.89 (m, 2H), 7.98 (d, J = 4.9 Hz, 1H), 7.36- 7.21 (m, 1H), 7.19- 6.94 (m, 1H), 2.83- 2.68 (m, 1H), 2.13- 2.06 (m, 1H), 1.66- 1.50 (m, 2H). | 413 |
| | 2-(4-fluoro-2-(4-(4- | | | |
| | (trifluoromethyl)pyridin- | | | |
| | 3-yl)-4H-1,2,4-triazol-3- | | | |
| | yl)-1H-benzo[d]imidazol- | | | |
| | 5-yl)cyclopropane-1- | | | |
| | carbonitrile | | | |
| Example 321 Method 8 | 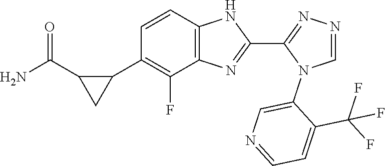 [SMILES: NC(=O)C1CC1c1ccc2[nH]c(-c3nncn3-c3cnccc3C(F)(F)F)nc2c1F] | white solid, yield: 33% | ¹H NMR (400 MHz, Methanol-d₄) δ 9.06 (t, J = 4.7 Hz, 1H), 9.00 8.89 (m, 2H), 7.97 (t, J = 4.3 Hz, 1H), 7.48- 6.99 (m, 2H), 2.57- 2.42 (m, 1H), 2.19- 2.07 (m, 1H), 1.93- 1.44 (m, 2H) | 432 |
| | 2-(4-fluoro-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazol-5- | | | |
| | yl)cyclopropane-1- | | | |
| | carboxamide | | | |
| Example 322 Method 2 | 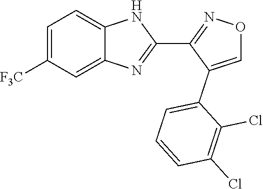 [SMILES: FC(F)(F)c1ccc2[nH]c(-c3nocc3-c3cccc(Cl)c3Cl)nc2c1] | white solid, yield: 16% | ¹H NMR (400 MHz, DMSO-d₆) δ 9.46 (s, 1H), 7.91 (s, 1H), 7.76 (dd, J = 8.0, 1.6 Hz, 2H), 7.55 (dd, J = 7.6, 1.2 Hz, 2H), 7.47 (t, J = 8.0 Hz, 1H). | 398 |
| | 4-(2,3-dichlorophenyl)- | | | |
| | 3-(5-(trifluoromethyl)- | | | |
| | 1H-benzo[d]imidazol-2- | | | |
| | yl)isoxazole | | | |
| Example 323 Method 10 | 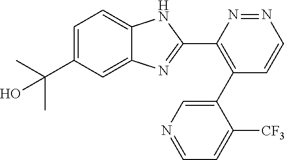 [SMILES: CC(C)(O)c1ccc2[nH]c(-c3nnccc3-c3cnccc3C(F)(F)F)nc2c1] | white solid, | ¹H NMR (400 MHz, DMSO) δ 14.07 (b, 1H), 9.55 (dd, J = 5.3, 1.8 Hz, 1H), 9.11 (s, 1H), 8.98 (d, J = 5.1 Hz, 1H), 7.95 (dd, J = 5.2, 1.7 Hz, 1H), 7.83-7.32 (m, 4H). | 400 |
| Example 324 Method 8 | 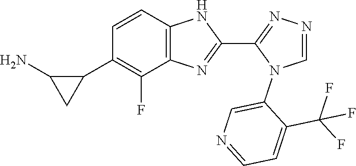 [SMILES: NC1CC1c1ccc2[nH]c(-c3nncn3-c3cnccc3C(F)(F)F)nc2c1F] | While solid, yield: 38%. | ¹H NMR (400 MHz, Methanol-d₄) δ 9.06 (d, J = 5.1 Hz, 1H), 8.98- 8.90 (m, 2H), 8.52 (s, 1H), 7.97 (d, J = 5.1 Hz, 1H), 7.33-6.87 (m, 2H), 2.86-2.75 (m, 1H), 2.42-2.30 (m, 1H), 2.21-2.00 (m, 1H), 1.61-1.37 (m, 1H). | 404 |
| | 2-(4-fluoro-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazol-5- | | | |
| | yl)cyclopropan-1-amine | | | |
| Example 325 Method 4 | 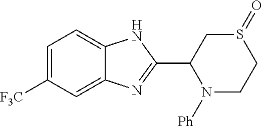 [SMILES: O=S1CCN(c2ccccc2)C(c2nc3cc(C(F)(F)F)ccc3[nH]2)C1] | Off- white solid, yield: 13.2% | ¹H NMR (400 MHz, DMSO-d₆) δ 12.88 (s, 1H), 8.05-7.57 (m, 2H), 7.48 (d, J = 8.5 Hz, 1H), 7.24 (dd, J = 8.7, 7.1 Hz, 2H), 7.14 (d, J = 8.1 Hz, 2H), 6.85 (t, J = 7.2 Hz, 1H), | 380 |
| | 4-phenyl-3-(5- | | 4.02 (dd, J = 15.4, | |
| | (trifluoromethyl)-1H- | | 5.9 Hz, 1H), 3.88-3.78 | |
| | benzo[d]imidazole-2- | | (m, 1H), 3.25 (s, 1H), | |
| | yl)thiomorpholine 1- | | 2.98 (dd, J = 12.9, | |
| | oxide | | 3.9 Hz, 1H), 2.68 (dt, | |
| | | | J = 8.9, 2.3 Hz, 1H). | |
| Example 326 Method 17 | 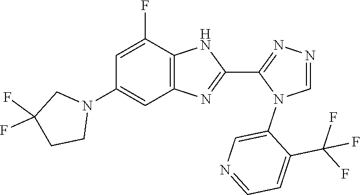 [SMILES: Fc1cc(N2CCC(F)(F)C2)cc2nc(-c3nncn3-c3cnccc3C(F)(F)F)[nH]c12] | white solid, yield: 16.9% | ¹H NMR (400 MHz, DMSO-d₆) δ 13.66- 13.30 (m, 1H), 9.10 (t, J = 5.2 Hz, 3H), 8.07 (d, J = 5.2 Hz, 1H), 6.46 (d, J = 13.6 Hz, 1H), 6.33 (d, J = 2.0 Hz, 1H), 3.71 (t, J = 13.2 Hz, 2H), 3.48 (t, J = 7.2 Hz, 2H), 2.61-2.52 (m, 2H). | 454 |
| | 5-(3,3- | | | |
| | difluoropyrrolidin-1- | | | |
| | yl)-7-fluoro-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazole | | | |
| Example 327 Method 7 | 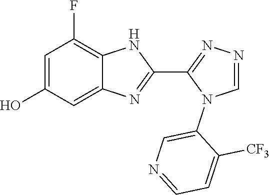 [SMILES: Oc1cc(F)c2[nH]c(-c3nncn3-c3cnccc3C(F)(F)F)nc2c1] | white solid, yield: 4.7% | ¹H NMR (400 MHz, DMSO-d₆) δ 13.45 (s, 1H), 9.77 (s, 1H), 9.06 (t, J = 4.6 Hz, 3H), 8.03 (d, J = 5.2 Hz, 1H), 6.63 (d, J = 2.0 Hz, 1H), 6.39 (dd, J = 12.4, 2.0 Hz, 1H). | 365 |
| | 7-fluoro-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazol-5-ol | | | |
| Example 328 Method 7 | 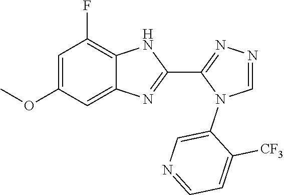 [SMILES: COc1cc(F)c2[nH]c(-c3nncn3-c3cnccc3C(F)(F)F)nc2c1] | white solid, yield: 18% | ¹H NMR (400 MHz, DMSO-d₆) δ 13.78 (s, 1H), 9.11 (d, J = 6.8 Hz, 3H), 8.08 (d, J = 5.0 Hz, 1H), 6.78 (s, 1H), 6.65 (d, J = 12.4 Hz, 1H), 3.79 (s, 3H). | 379 |
| | 7-fluoro-5-methoxy-2- | | | |
| | (4-(4- | | | |
| | (trifluoromethyl)pyridin- | | | |
| | 3-yl)-4H-1,2,4-triazol- | | | |
| | 3-yl)-1H- | | | |
| | benzo[d]imidazole | | | |
| Example 329 Method 8 | 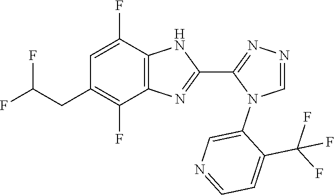 [SMILES: Fc1c(CC(F)F)cc(F)c2[nH]c(-c3nncn3-c3cnccc3C(F)(F)F)nc12] | white solid, yield: 2.14% | ¹H NMR (400 MHz, DMSO-d₆) δ 14.69 (s, 1H), 9.20 (d, J = 11.5 Hz, 1H), 9.15-9.08 (m, 2H), 8.10-8.07 (m, 1H), 7.76-7.63 (m, 1H), 6.27 (td, J = 56.4, 13.0 Hz, 1H), 3.28-3.15 (m, 2 H). | 431 |
| | 5-(2,2-difluoroethyl)- | | | |
| | 4,7-difluoro-2-[4-[4- | | | |
| | (trifluoromethyl)-3- | | | |
| | pyridyl]-1,2,4-triazol-3- | | | |
| | yl]-1H-benzimidazole | | | |
| Example 330 Method 9 | 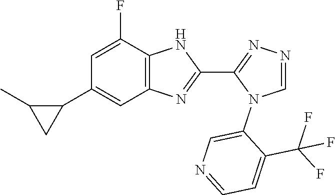 [SMILES: CC1CC1c1cc(F)c2[nH]c(-c3nncn3-c3cnccc3C(F)(F)F)nc2c1] | white solid, yield: 23.8% | ¹H NMR (400 MHz, DMSO-d₆) δ 13.83 (br s, 1H), 9.17-9.00 (m, 3H), 8.08 (d, J = 5.2 Hz, 1H), 7.08 (s, 1H), 6.82 (d, J = 12.0 Hz, 1H), 2.17 (m, 1H), 1.13 (m, 1H), 0.97 (m, 1H), 0.71 (d, J = 6.0 Hz, 3H), 0.66 (m, 1H). | 403 |
| | 7-fluoro-5-(2- | | | |
| | methylcyclopropyl)-2- | | | |
| | (4-(4- | | | |
| | (trifluoromethyl)pyridin- | | | |
| | 3-yl)-4H-1,2,4-triazol- | | | |
| | 3-yl)-1H- | | | |
| | benzo[d]imidazole | | | |
| Example 331 Method 7 | 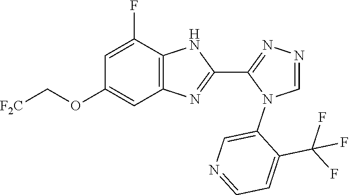 | white solid, yield: 16% | ¹H NMR (400 MHz, DMSO-d₆) δ 13.89 (s, 1H), 9.14-9.10 (m, 3H), 8.08 (d, J = 5.0 Hz, 1H), 6.87 (s, 1H), 6.76 (d, J = 12.2 Hz, 1H), 6.39 (s, 1H), 4.37 (t, J = 14.2 Hz, 2H). | 428 |
| | 5-(2,2-difluoroethoxy)- | | | |
| | 7-fluoro-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazole | | | |
| Example 332 Method 9 | 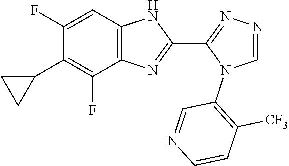 [SMILES: Fc1cc2[nH]c(-c3nncn3-c3cnccc3C(F)(F)F)nc2c(F)c1C1CC1] | white solid, | ¹H NMR (400 MHz, DMSO-d₆) δ 9.21- 8.94 (m, 3H), 8.08 (d, J = 5.2 Hz, 1H), 7.08 (d, J = 9.9 Hz, 1H), 1.85-1.75 (m, 1H), 1.00-0.88 (m, 2H), 0.85-0.72 (m, 2H). | 407 |
| | 5-cyclopropyl-4,6- | | | |
| | difluoro-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazole | | | |
| Example 333 Method 10 | 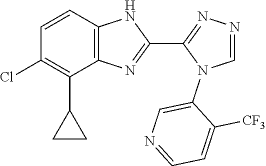 [SMILES: FC(F)(F)c1ccncc1-n1cnnc1-c1nc2c(C3CC3)c(Cl)ccc2[nH]1] | white solid, yield: 6.5% | ¹H NMR (400 MHz, DMSO-d₆) δ 13.77 (s, 1H), 9.16-9.05 (m, 3H), 8.14 (d, J = 5.2 Hz, 1H), 7.25 (s, 2H), 2.01-1.96 (m, 1H), 0.82-0.63 (m, 4H). | 405 |
| | 6-chloro-7-cyclopropyl- | | | |
| | 2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazole | | | |
| Example 334 Method 10 | 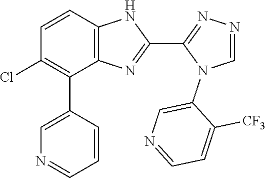 [SMILES: FC(F)(F)c1ccncc1-n1cnnc1-c1nc2c(-c3cccnc3)c(Cl)ccc2[nH]1] | white solid, yield: 12.8% | ¹H NMR (400 MHz, DMSO-d₆) δ 14.02 (s, 1H), 9.13 (s, 1H), 8.96- 8.87 (m, 2H), 8.56 (d, J = 4.8 Hz, 1H), 8.37 (s, 1H), 7.90 (d, J = 5.2 Hz, 1H), 7.55 (d, J = 8.8 Hz, 1H), 7.47 (d, J = 8.4 Hz, 2H), 7.42-7.37 (m, 1H). | 442 |
| | 6-chloro-7-(pyridin-3- | | | |
| | yl)-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazole | | | |
| Example 335 Method 2 | 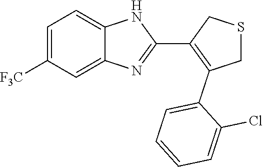 [SMILES: FC(F)(F)c1ccc2[nH]c(C3=C(c4ccccc4Cl)CSC3)nc2c1] | white solid | ¹H NMR (400 MHz, Methanol-d₄) δ 7.76 (s, 1H), 7.57 (s, 1H), 7.51- 7.41 (m, 2H), 7.41- 7.28 (m, 3H), 4.52- 4.44 (m, 2H), 4.29- 4.24 (m, 2H). | 381 |
| | 2-(4-(2-chlorophenyl)- | | | |
| | 2,5-dihydrothiophen-3- | | | |
| | yl)-5-(trifluoromethyl)- | | | |
| | 1H-benzo[d]imidazole | | | |
| Example 336 Method 10 | 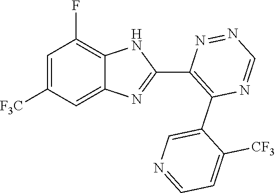 [SMILES: Fc1cc(C(F)(F)F)cc2nc(-c3nncnc3-c3cnccc3C(F)(F)F)[nH]c12] | white solid, yield: 27%. | ¹H NMR (400 MHz, DMSO-d₆) δ: 14.66 (s, 1H), 10.15 (s, 1H), 9.07 (d, J = 5.2 Hz, 1H), 8.93 (s, 1H), 8.00 (d, J = 5.2 Hz, 1H), 7.70 (s, 1H), 7.42 (d, J = 10.0 Hz, 1H). | 427 [M-H]- |
| | 7-fluoro-5- | | | |
| | (trifluoromethyl)-2-(5- | | | |
| | (4- | | | |
| | (trifluoromethyl)pyridin- | | | |
| | 3-yl)-1,2,4-triazin-6- | | | |
| | yl)-1H- | | | |
| | benzo[d]imidazole | | | |
| Example 337 Method 2 | 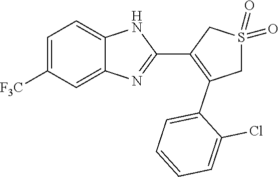 [SMILES: O=S1(=O)CC(c2nc3cc(C(F)(F)F)ccc3[nH]2)=C(c2ccccc2Cl)C1] | white solid, yield: 17.5% | ¹H NMR (400 MHz, Methanol-d₄) δ 7.93- 7.65 (m, 2H), 7.55- 7.47 (m, 2H), 7.46- 7.41 (m, 1H), 7.38- 7.33 (m, 2H), 4.63- 74.61 (m, 2H), 4.47- 4.44 (m, 2H). | 413 |
| | Preparation of 3-(2- | | | |
| | chlorophenyl)-4-(5- | | | |
| | (trifluoromethyl)-1H- | | | |
| | benzo[d]imidazol-2-yl)- | | | |
| | 2,5-dihydrothiophene | | | |
| | 1,1-dioxide | | | |
| Example 339 Method 13 | 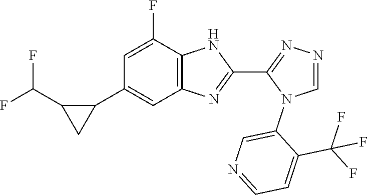 [SMILES: Fc1cc(C2CC2C(F)F)cc2nc(-c3nncn3-c3cnccc3C(F)(F)F)[nH]c12] | white solid, yield: 58.33% | ¹H NMR (400 MHz, DMSO-d₆) δ 13.89 (d, J = 18.4 Hz, 1H), 9.17- 9.07 (m, 3H), 8.08 (dd, J = 5.2, 3.4 Hz, 1H), 7.21 (d, 1H), 6.84 (dd, J = 59.9, 12.0 Hz, 1H), 6.11-4.97 (m, 3H), 1.79-1.69 (m, 1H), 1.53-1.34 (m, 1H), 1.24-1.08 (m, 2H). | 439 |
| | 5-(2- | | | |
| | (difluoromethyl)cyclo- | | | |
| | propyl)-7-fluoro-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazole | | | |
| Example 340 Method 4 | 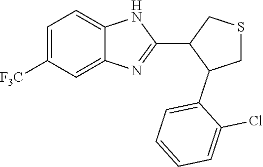 [SMILES: FC(F)(F)c1ccc2[nH]c(C3CSCC3c3ccccc3Cl)nc2c1] | white solid, yield: 39.8% | ¹H NMR (400 MHz, Methanol-d₄) δ 7.62 (s, 1H), 7.44 (s, 1H), 7.34 (d, J = 8.4 Hz, 1H), 7.16 (d, J = 8.0 Hz, 1H), 7.10 (d, J = 8.0 Hz, 1H), 7.02 (t, J = 7.6 Hz, 1H), 6.94 (t, J = 7.6 Hz, 1H), 4.39-4.33 (m, 1H), 4.14-4.09 (m, 1H), | 383 |
| | 2-(4-(2- | | 3.41-3.31 (m, 4H). | |
| | chlorophenyl)tetrahydro | | | |
| | thiophen-3-yl)-5- | | | |
| | (trifluoromethyl)-1H- | | | |
| | benzo[d]imidazole | | | |
| Example 341 Method 17 | 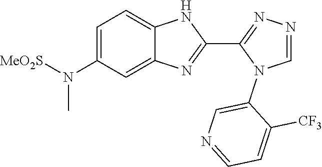 [SMILES: CN(c1ccc2[nH]c(-c3nncn3-c3cnccc3C(F)(F)F)nc2c1)S(C)(=O)=O] | white solid, yield: 48% | ¹H NMR (500 MHz, CDCl₃) δ 9.12 (d, J = 5.2 Hz, 1H), 8.89 (s, 1H), 8.48 (s, 1H), 7.88 (d, J = 5.0 Hz, 1H), 7.83-7.61 (m, 1H), 7.34 (br s, 1H), 7.27 (s, 1H), 3.37 (s, 3H), 2.88 (s, 3H) | 438 |
| | N-methyl-N-[2-[4-[4- | | | |
| | (trifluoromethyl)-3- | | | |
| | pyridyl]-1,2,4-triazol-3- | | | |
| | yl]-1H-benzimidazol-5- | | | |
| | yl]methanesulfonamide | | | |
| Example 342 Method 17 | 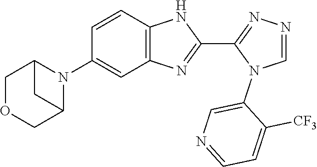 [SMILES: FC(F)(F)c1ccncc1-n1cnnc1-c1nc2cc(N3C4COCC3C4)ccc2[nH]1] | white solid, yield: 39% | ¹H NMR (400 MHz, CDCl₃) δ 9.06 (d, J = 5.1 Hz, 1H), 8.86 (s, 1H), 8.35 (s, 1H), 7.81 (d, J = 5.0 Hz, 1H), 7.37-7.28 (m, 1H), 6.82 (br s, 1H), 6.49 (br s, 1H), 4.45 (br d, J = 9.3 Hz, 2H), 4.30 (br s, 2H), 3.71 (br d, J = | 428 |
| | 6-[2-[4-[4- | | 10.4 Hz, 2H), 2.84 | |
| | (trifluoromethyl)-3- | | (br d, J = 6.1 Hz, 1H), | |
| | pyridyl]-1,2,4-triazol-3- | | 2.11-1.99 (m, 1H) | |
| | yl]-1H-benzimidazol-5- | | | |
| | yl]-3-oxa-6- | | | |
| | azabicyclo[3.1.1]heptane | | | |
| Example 343 Method 10 | 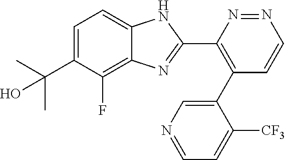 [SMILES: CC(C)(O)c1ccc2[nH]c(-c3nnccc3-c3cnccc3C(F)(F)F)nc2c1F] | White solid | ¹H NMR (400 MHz, DMSO) δ 13.72 (b, 1H), 9.50 (d, J = 5.2 Hz, 1H), 8.98 (d, J = 5.2 Hz, 1H), 8.75 (s, 1H), 7.94 (dd, J = 12.8, 5.2 Hz, 2H), 7.53 (t, J = 7.8 Hz, 1H), 7.26 (d, J = 8.5 Hz, 1H), 5.22 (s, 1H), 1.47 (d, J = | 418 |
| | 2-(4-Fluoro-2-(4-(4- | | 4.3 Hz, 6H). | |
| | (trifluoromethyl)pyridin- | | | |
| | 3-yl)pyridazin-3-yl)- | | | |
| | 1H-benzo[d]imidazol-5- | | | |
| | yl)propan-2-ol | | | |
| Example 344 Method 10 | 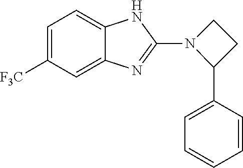 [SMILES: FC(F)(F)c1ccc2[nH]c(N3CCC3c3ccccc3)nc2c1] | White solid | ¹H NMR (400 MHz, DMSO-d₆) δ 11.73 (b, 1H), 7.53-7.18 (m, 8H), 5.41 (t, J = 7.9 Hz, 1H), 4.13 (td, J = 14.6, 6.5 Hz, 2H), 2.88-2.75 (m, 1H), 2.36-2.23 (m, 1H). | 318 |
| | 2-(2-phenylazetidin-1-yl)- | | | |
| | 5-(trifluoromethyl)-1H- | | | |
| | benzo[d]imidazole | | | |
| Example 345 Method 4 | 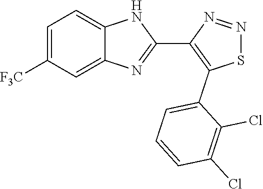 [SMILES: FC(F)(F)c1ccc2[nH]c(-c3nnsc3-c3cccc(Cl)c3Cl)nc2c1] | White solid, yield: 25.6% | ¹H NMR (400 MHz, Chloroform-d) δ 10.50 (s, 1H), 8.06-7.31 (m, 6H). | 414 |
| | 5-(2,3-dichlorophenyl)- | | | |
| | 4-(5-(trifluoromethyl)- | | | |
| | 1H-benzo[d]imidazol-2- | | | |
| | yl)isothiazole | | | |
| Example 346 Method 18 | 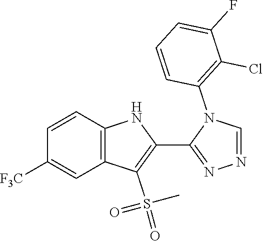 [SMILES: CS(=O)(=O)c1c(-c2nncn2-c2cccc(F)c2Cl)[nH]c2ccc(C(F)(F)F)cc12] | pale yellow solid, yield: 11% | ¹H NMR (400 MHz, DMSO-d₆) δ: 13.38 (s, 0.5H), 9.22 (s, 1H), 8.19 (s, 1H), 7.83-7.35 (m, 5H), 3.06 (s, 3H). | 459 |
| | 2-[4-(2-chloro-3- | | | |
| | fluorophenyl)-1,2,4- | | | |
| | triazol-3-yl]-3- | | | |
| | methanesulfonyl-5- | | | |
| | (trifluoromethyl)-1H- | | | |
| | indole | | | |
| Example 347 Method 9 | 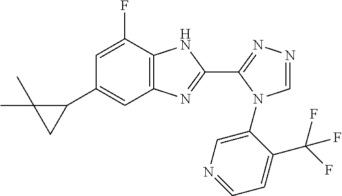 [SMILES: CC1(C)CC1c1cc(F)c2[nH]c(-c3nncn3-c3cnccc3C(F)(F)F)nc2c1] | white solid, yield: 52.5% | ¹H NMR (400 MHz, DMSO-d₆) δ 13.82 (br s, 1H), 9.24-8.97 (m, 3H), 8.08 (d, J = 5.2 Hz, 1H), 7.04 (s, 1H), 6.80 (d, J = 12.0 Hz, 1H), 1.97 (m, 1H), 1.19 (s, 3H), 0.87 (m, 1H), 0.77 (m, 1H), 0.72 (s, 3H). | 417 |
| | 5-(2,2- | | | |
| | dimethylcyclopropyl)- | | | |
| | 7-fluoro-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazole | | | |
| Example 348 Method 10 | 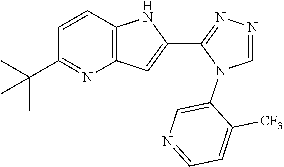 [SMILES: CC(C)(C)c1ccc2[nH]c(-c3nncn3-c3cnccc3C(F)(F)F)cc2n1] | white solid, yield: 21.1% | ¹H NMR (400 MHz, DMSO-d₆) δ 12.21 (br s, 1H), 9.22 (d, J = 5.2 Hz, 1H), 9.19 (s, 1H), 9.02 (s, 1H), 8.19 (d, J = 5.2 Hz, 1H), 7.72 (d, J = 8.8 Hz, 1H), 7.29 (d, J = 8.8 Hz, 1H), 5.60-5.44 (s, 1H), 1.28 (s, 9H). | 387 |
| | 5-(tert-butyl)-2-(4-(4- | | | |
| | (trifluoromethyl)pyridin- | | | |
| | 3-yl)-4H-1,2,4-triazol- | | | |
| | 3-yl)-1H-pyrrolo[3,2- | | | |
| | b]pyridine | | | |
| Example 349 Method 19 single unknown stereoisomer | 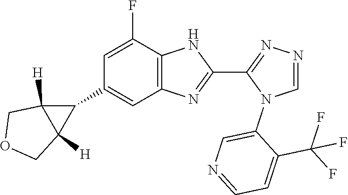 [SMILES: Fc1cc([C@@H]2[C@@H]3COC[C@@H]32)cc2nc(-c3nncn3-c3cnccc3C(F)(F)F)[nH]c12] | white solid, yield: 9.5% | ¹H NMR (CDCl₃, 400 MHz) δ ppm 11.30- 11.60 (m, 1H), 9.08 (d, 1H, J = 5.2 Hz), 8.90 (br s, 1H), 8.39 (s, 1H), 7.81 (d, 1H, J = 5.2 Hz), 7.19 (br s, 1H), 6.70-6.80 (m, 1H), 4.10-4.20 (m, 1H), 4.0- 4.1 (m, 1H), 3.80-4.00 (m, 2H), 2.20-2.30 (m, 1H), 2.00-2.10 (m, 2H). | 431 |
| | 7-fluoro-5-(3- | | | |
| | oxabicyclo[3.1.0]hexan- | | | |
| | 6-yl)-2-[4-[4- | | | |
| | (trifluoromethyl)-3- | | | |
| | pyridyl]-1,2,4-triazol-3- | | | |
| | yl]-1H-benzimidazole | | | |
| Example 350 Method 2 single unknown stereoisomer | 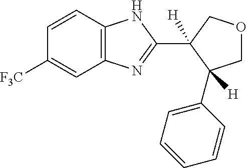 [SMILES: FC(F)(F)c1ccc2[nH]c([C@H]3COC[C@@H]3c3ccccc3)nc2c1] | white solid, | ¹H NMR (400 MHz, Chloroform-d) δ 9.94- 9.17 (m, 1H), 7.88 (s, 1H), 7.69 (d, J = 8.7 Hz, 1H), 7.55 (d, J = 8.3 Hz, 1H), 7.14 (d, J = 7.5 Hz, 2H), 7.03 (m, 3H), 4.64 (m, 2H), 4.38 (m 1H), 4.30 (m, 2H), 4.17 (m, 1H). | 333 |
| | 2-((3S,4S)-4- | | | |
| | phenyltetrahydrofuran- | | | |
| | 3-yl)-5- | | | |
| | (trifluoromethyl)-1H- | | | |
| | benzo[d]imidazole | | | |
| Example 351 Method 2 single unknown stereoisomer | 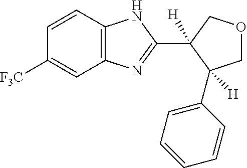 [SMILES: FC(F)(F)c1ccc2[nH]c([C@H]3COC[C@H]3c3ccccc3)nc2c1] | white solid, | ¹H NMR (400 MHz, Chloroform-d) δ 8.01 (s, 1H), 7.81 (d, J = 8.7 Hz, 1H), 7.65 (dd, J = 8.8, 1.6 Hz, 1H), 7.29 (s, 1H), 7.26-7.11 m, 5H), 4.38 (dt, J = 16.7, 7.4 Hz, 2H), 4.32- 4.17 (m, 2H), 4.06 (q, J = 8.0 Hz, 1H), 3.84 (t, J = 8.4 Hz, 1H). | 333 |
| | 2-((3S,4R)-4- | | | |
| | phenyltetrahydrofuran- | | | |
| | 3-yl)-5- | | | |
| | (trifluoromethyl)-1H- | | | |
| | benzo[d]imidazole | | | |
| Example 352 Method 8 | 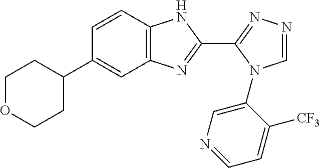 [SMILES: FC(F)(F)c1ccncc1-n1cnnc1-c1nc2cc(C3CCOCC3)ccc2[nH]1] | white solid, yield: 47% | ¹H NMR (400 MHz, CDCl₃) δ 12.19-11.95 (m, 1H), 9.09 (t, J = 5.5 Hz, 1H), 8.88 (d, J = 2.7 Hz, 1H), 8.42 (s, 1H), 7.84 (t, J = 5.5 Hz, 1H), 7.70-7.41 (d, J = 8.5 Hz, 1H), 7.24-7.11 (d, J = 9.7 Hz, 1H), 4.14-4.06 (m, 2H), 3.61-3.49 (m, 2H), | 415 |
| | 5-tetrahydropyran-4-yl- | | 2.94-2.79 (m, 1H), | |
| | 2-[4-[4- | | 1.99-1.76 (m, 4H) | |
| | (trifluoromethyl)-3- | | | |
| | pyridyl]-1,2,4-triazol-3- | | | |
| | yl]-1H-benzimidazole | | | |
| Example 353 Method 13 | 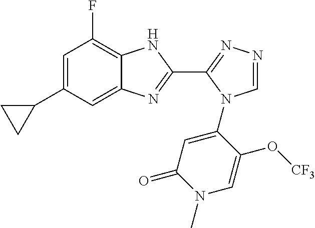 [SMILES: Cn1cc(OC(F)(F)F)c(-n2cnnc2-c2nc3cc(C4CC4)cc(F)c3[nH]2)cc1=O] | White solid, yield: 16% | ¹H NMR (400 MHz, DMSO-d₆) δ: 13.73 (s, 1H), 9.04 (s, 1H), 8.41 (s, 1H), 7.09 (s, 1H), 7.02 (s, 1H), 6.88 (d, J = 12.4 Hz, 1H), 3.55 (s, 3H), 2.09-2.02 (m, 1H), 0.99-0.95 (m, 2H), 0.72-0.69 (m, 2H). | 435 |
| | 4-(3-(5-cyclopropyl-7- | | | |
| | fluoro-1H- | | | |
| | benzo[d]imidazol-2-yl)- | | | |
| | 4H-1,2,4-triazol-4-yl)- | | | |
| | 1-methyl-5- | | | |
| | (trifluoromethoxy)pyridin- | | | |
| | 2(1H)-one | | | |
| Example 354 Method 13 | 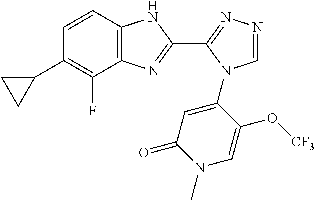 [SMILES: Cn1cc(OC(F)(F)F)c(-n2cnnc2-c2nc3c(F)c(C4CC4)ccc3[nH]2)cc1=O] | White solid, yield: 16% | ¹H NMR (400 MHz, DMSO-d₆) δ: 9.06 (s, 1H), 8.41 (s, 1H), 7.26 (d, J = 8.4 Hz, 1H), 7.03 (s, 1H), 6.89-6.85 (m, 1H), 3.55 (s, 3H), 2.13-2.10 (m, 1H), 0.99-0.95 (m, 2H), 0.73-0.69 (m, 2H). | 435 |
| | 4-(3-(5-cyclopropyl-4- | | | |
| | fluoro-1H- | | | |
| | benzo[d]imidazol-2-yl)- | | | |
| | 4H-1,2,4-triazol-4-yl)- | | | |
| | 1-methyl-5- | | | |
| | (trifluoromethoxy) | | | |
| | pyridin-2(1H)-one | | | |
| Example 355 Method 7 | 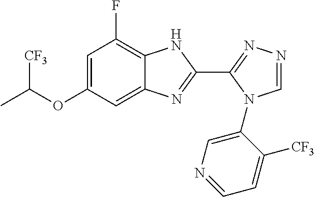 [SMILES: CC(Oc1cc(F)c2[nH]c(-c3nncn3-c3cnccc3C(F)(F)F)nc2c1)C(F)(F)F] | white solid, yield: 38.5% | ¹H NMR (400 MHz, DMSO-d₆) δ 13.92 (br s, 1H), 9.30-8.96 (m, 3H), 8.07 (d, J = 5.2 Hz, 1H), 6.97 (d, J = 2.2 Hz, 1H), 6.84 (dd, J = 12.0, 2.2 Hz, 1H), 5.27 (m, 1H), 1.39 (d, J = 6.4 Hz, 3H). | 461 |
| | 7-fluoro-2-(4-(4- (trifluoromethyl)pyridin- 3-yl)-4H-1,2,4-triazol- 3-yl)-5-((1,1,1- trifluoropropan-2- yl)oxy)-1H- benzo[d]imidazole | | | |
| Example 356 Method 10 | 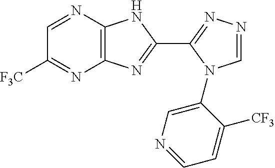 [SMILES: FC(F)(F)c1cnc2[nH]c(-c3nncn3-c3cnccc3C(F)(F)F)nc2n1] | white solid, yield: 12.1% | ¹H NMR (400 MHz, Methanol-d₄) δ 9.21 (d, J = 5.2 Hz, 1H), 9.10 (s, 1H), 9.01 (s, 1H), 8.76 (s, 1H), 8.12 (d, J = 5.2 Hz, 1H), 5.92 (d, J = 1.2 Hz, 1H). | 400 |
| | 2-(trifluoromethyl)-6- | | | |
| | (4-(4- | | | |
| | (trifluoromethyl)pyridin- | | | |
| | 3-yl)-4H-1,2,4-triazol- | | | |
| | 3-yl)-5H-pyrrolo[2,3- | | | |
| | b]pyrazine | | | |
| Example 357 Method 13 | 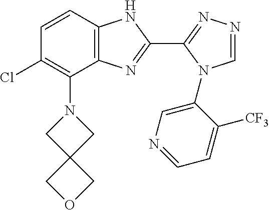 [SMILES: FC(F)(F)c1ccncc1-n1cnnc1-c1nc2c(N3CC4(COC4)C3)c(Cl)ccc2[nH]1] | white solid, yield: 13.3% | ¹H NMR (400 MHz, DMSO-d₆) δ 13.59 (s, 1H), 9.19-9.08 (m, 3H), 8.21 (d, J = 5.2 Hz, 1H), 7.02 (d, J = 8.4 Hz, 1H), 6.77 (d, J = 8.4 Hz, 1H), 4.61 (d, J = 2.4 Hz, 4H), 4.02 (s, 4H). | 462 |
| | 6-(5-chloro-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazol-4-yl)- | | | |
| | 2-oxa-6- | | | |
| | azaspiro[3.3]heptane | | | |
| Example 358 Method 13 | 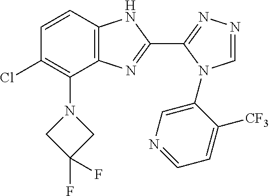 [SMILES: FC1(F)CN(c2c(Cl)ccc3[nH]c(-c4nncn4-c4cnccc4C(F)(F)F)nc23)C1] | white solid, yield: 4.1% | ¹H NMR (400 MHz, DMSO-d₆) δ 9.16 (s, 1H), 9.12 (s, 1H), 9.09 (d, J = 5.2 Hz, 1H), 8.15 (d, J = 5.0 Hz, 1H), 7.12 (d, J = 8.4 Hz, 1H), 6.93 (d, J = 8.4 Hz, 1H), 4.25 (td, J = 12.8, 3.2 Hz, 4H). | 456 |
| | 5-chloro-4-(3,3- | | | |
| | difluoroazetidin-1-yl)- | | | |
| | 2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazole | | | |
| Example 359 Method 8 | 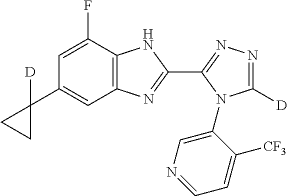 [SMILES: [2H]c1nnc(-c2nc3cc(C4([2H])CC4)cc(F)c3[nH]2)n1-c1cnccc1C(F)(F)F] | white solid, yield: 17.3% | ¹H NMR (400 MHz, Methanol-d₄) δ 9.06 (s, 1H), 8.96 (s, 1H), 7.98 (d, J = 5.2 Hz, 1H), 7.08 (s, 1H), 6.63 (d, J = 12.0 Hz, 1H), 1.05- 0.89 (m, 2H), 0.75- 0.64 (m, 2H). | 391 |
| | 5-(cyclopropyl-1-d)-7- | | | |
| | fluoro-2-(4-(4- | | | |
| | (trifluoromethyl)pyridin- | | | |
| | 3-yl)-4H-1,2,4-triazol- | | | |
| | 3-yl-5-d)-1H- | | | |
| | benzo[d]imidazole | | | |
| Example 360 Method 19 | 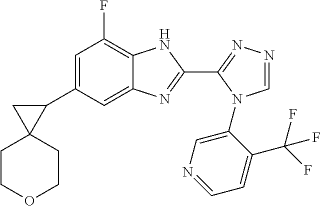 [SMILES: Fc1cc(C2CC23CCOCC3)cc2nc(-c3nncn3-c3cnccc3C(F)(F)F)[nH]c12] | white solid, yield: 8.6% | ¹H NMR (500 MHz,CDCl₃) δ 9.11 (d, J = 5.2 Hz, 1H), 8.89- 8.85 (m, 1H), 8.45 (s, 1H), 7.86 (d, J = 5.0 Hz, 1H), 7.40-7.27 (m, 2H), 6.85 (br d, J = 9.6 Hz, 1H), 3.89-3.74 (m, 2H), 3.59-3.49 (m, 2H), 2.09 (br t, J = 6.7 Hz, 1H), 1.73-1.61 (m, 1H), 1.59-1.50 (m, 1H), 1.33-1.23 (m, | 459 |
| | 7-fluoro-5-(6- | | 1H), 1.19 (br s, 1H), | |
| | oxaspiro[2.5]octan-2- | | 0.99 (br s, 1H), 0.96- | |
| | yl)-2-[4-[4- | | 0.88 (m, 1H) | |
| | (trifluoromethyl)-3- | | | |
| | pyridyl]-1,2,4-triazol-3- | | | |
| | yl]-1H-benzimidazole | | | |
| Example 361 Method 4 | 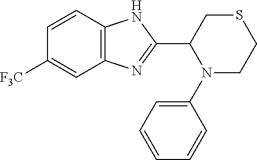 [SMILES: FC(F)(F)c1ccc2[nH]c(C3CSCCN3c3ccccc3)nc2c1] | white solid, yield: 29% | ¹H NMR (CHLOROFORM-d, 400 MHz) δ 9.60-9.80 (m, 1H), 7.80-8.10 (m, 1H), 7.40-7.70 (m, 2H), 7.30-7.30 (m, 2H), 7.00-7.10 (m, 2H), 6.94 (t, 1H, J = 7.3 Hz), 5.34 (t, 1H, J = 4.5 Hz), 3.80-3.80 (m, 1H), | 364 |
| | 4-phenyl-3-[5- | | 3.60-3.70 (m, 1H), | |
| | (trifluoromethyl)-1H- | | 3.30-3.40 (m, 1H), | |
| | benzimidazol-2- | | 3.30-3.30 (m, 1H), 2.99 | |
| | yl]thiomorpholine | | (ddd, 1H, J = 2.8, 9.9, | |
| | | | 13.1 Hz), 2.60-2.70 (m, | |
| | | | 1H). | |
| Example 362 Method 19 | 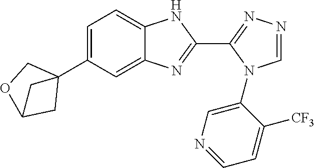 [SMILES: FC(F)(F)c1ccncc1-n1cnnc1-c1nc2cc(C34COC(C3)C4)ccc2[nH]1] | white solid, yield: 17% | ¹H NMR (CHLOROFORM-d, 400 MHz) δ 12.00- 12.30 (m, 1H), 9.10 (t, 1H, J = 5.3 Hz), 8.89 (s, 1H), 8.43 (d, 1H, J = 2.9 Hz), 7.70-7.90 (m, 2H), 7.30-7.50 (m, 1H), 7.10-7.30 (m, 1H), 4.68 (d, 1H, J = 11.4 Hz), | 413 |
| | 5-(2- | | 3.91 (s, 1H), 3.85 (s, | |
| | oxabicyclo[2.1.1]hexan- | | 1H), 2.1-1.90 (m, 4H). | |
| | 4-yl)-2-[4-[4- | | | |
| | (trifluoromethyl)-3- | | | |
| | pyridyl]-1,2,4-triazol-3- | | | |
| | yl]-1H-benzimidazole | | | |
| Example 363 Method 4 | 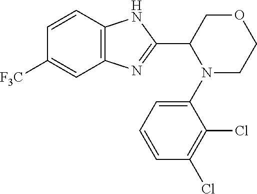 [SMILES: FC(F)(F)c1ccc2[nH]c(C3COCCN3c3cccc(Cl)c3Cl)nc2c1] | White solid, yield: 6.5% | ¹H NMR (400 MHz, CDCl₃) δ 7.83 (s, 1H), 7.59 (s, 1H), 7.48 (d, J = 8.2 Hz, 1H), 7.21- 7.16 (m, 1H), 7.08- 7.03 (m, 2H), 4.97- 4.90 (m, 1H), 4.31- 4.26 (m, 1H), 4.08- 3.97 (m, 2H), 3.93- 3.82 (m, 1H), 3.43- 3.35 (m, 1H), 2.95- 2.85 (m, 1H). | 416 |
| | 4-(2,3-dichlorophenyl)- | | | |
| | 3-[5-(trifluoromethyl)- | | | |
| | 1H-benzimidazol-2- | | | |
| | yl]morpholine | | | |
| Example 364 Method 8 | 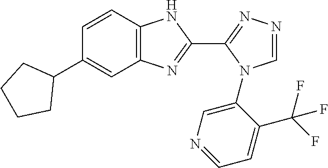 [SMILES: FC(F)(F)c1ccncc1-n1cnnc1-c1nc2cc(C3CCCC3)ccc2[nH]1] | white solid, yield: 32.65% | ¹H NMR (400 MHz, Methanol-d₄) δ 9.06 (d, J = 5.2 Hz, 1H), 8.95 (s, 1H), 8.90 (s, 1H), 7.97 (d, J = 5.2 Hz, 1H), 7.48-7.38 (m, 1H), 7.35-7.08 (m, 2H), 3.14-3.03 (m, 1H), 2.15-2.00 (m, 2H), 1.89-1.77 (m, 2H), 1.74-1.56 (m, 4H). | 399 |
| | 5-cyclopentyl-2-(4-(4- | | | |
| | (trifluoromethyl)pyridin- | | | |
| | 3-yl)-4H-1,2,4-triazol-3- | | | |
| | yl)-1H-benzo[d]imidazole | | | |
| Example 365 Method 13 | 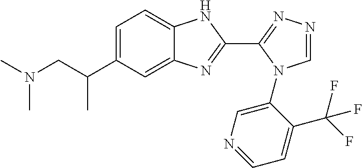 [SMILES: CC(CN(C)C)c1ccc2[nH]c(-c3nncn3-c3cnccc3C(F)(F)F)nc2c1] | white solid, | ¹H NMR (400 MHz, MeOD-d₄) δ 9.07 (d, J = 5.1 Hz, 1H), 8.97-8.90 (m, 2H), 7.98 (d, J = 5.1 Hz, 1H), 7.44-7.36 (m, 2H), 7.16 (dd, J = 1.4, 8.4 Hz, 1H), 3.09-3.01 (m, 1H), 2.62-2.46 (m, 2H), 2.20 (s, 6H), 1.26 (d, J = 6.9 Hz, 3H) | 413 |
| | 2-(2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazol-5- | | | |
| | yl)propan-1-amine | | | |
| Example 366 Method 19 | 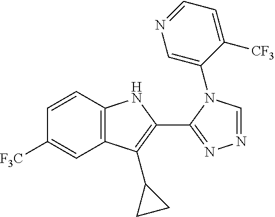 [SMILES: FC(F)(F)c1ccc2[nH]c(-c3nncn3-c3cnccc3C(F)(F)F)c(C3CC3)c2c1] | white solid, yield: 11% | ¹H NMR (400 MHz, MeOD) δ 9.01 (s, 1H), 8.99 (s, 1H), 8.95 (d, J = 5.1 Hz, 1H), 7.85 (d, J = 4.7 Hz, 2H), 7.51- 7.40 (m, 2H), 1.35- 1.28 (m, 1H), 0.83 (br d, J = 7.6 Hz, 2H), 0.53 (br d, J = 3.5 Hz, 2H) | 438 |
| | 3-cyclopropyl-5- | | | |
| | (trifluoromethyl)-2-(4- | | | |
| | (4- | | | |
| | (trifluoromethyl)pyridin- | | | |
| | 3-yl)-4H-1,2,4-triazol- | | | |
| | 3-yl)-1H-indole | | | |
| Example 367 Method 19 | 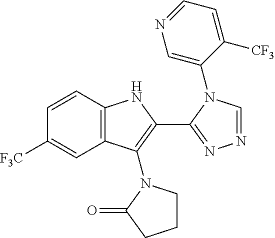 [SMILES: O=C1CCCN1c1c(-c2nncn2-c2cnccc2C(F)(F)F)[nH]c2ccc(C(F)(F)F)cc12] | white solid, yield: 36% | ¹H NMR (400 MHz, MeOD) δ 8.99 (d, J = 5.0 Hz, 1H), 8.83 (s, 1H), 8.27 (br s, 1H), 7.97-7.91 (m, 2H), 7.53 (d, J = 8.7 Hz, 1H), 7.09 (d, J = 8.7 Hz, 1H), 4.09- 3.93 (m, 2H), 2.74- 2.59 (m, 2H), 2.37 (quin, J = 7.4 Hz, 2H) | 481 |
| | 1-(5-(trifluoromethyl)-2- | | | |
| | (4-(4- | | | |
| | (trifluoromethyl)pyridin- | | | |
| | 3-yl)-4H-1,2,4-triazol-3- | | | |
| | yl)-1H-indol-3- | | | |
| | yl)pyrrolidin-2-one | | | |
| Example 368 Method 4 | 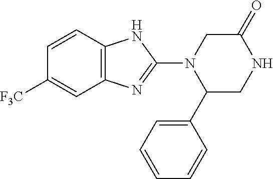 [SMILES: O=C1CN(c2nc3cc(C(F)(F)F)ccc3[nH]2)C(c2ccccc2)CN1] | | 1H NMR (CHLOROFORM-d, 400 MHz) δ 7.57 (m, 1H), 7.5-7.3 (m, 8H), 6.18 (m, 1H), 5.62 (m, 1H), 4.57-4.53 (m, 1H), 4.29 (m, 1H,), 4.08 (m, 1H), 3.82 (m, 1H) | 361 |
| | 5-phenyl-4-(5- | | | |
| | (trifluoromethyl)-1H- | | | |
| | benzo[d]imidazol-2- | | | |
| | yl)piperazin-2-one | | | |
| Example 369 Method 4 | 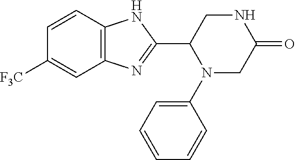 [SMILES: O=C1CN(c2ccccc2)C(c2nc3cc(C(F)(F)F)ccc3[nH]2)CN1] | white solid, yield: 24% | ¹H NMR (METHANOL-d₄, 400 MHz,) δ = 7.51 (d, J = 8.0 Hz, 1H), 7.25 (t, J = 7.7 Hz, 2H), 6.87-6.78 (m, 4H), 5.31 (t, J = 3.4 Hz, 1H), 4.18 (s, 2H), 4.04-3.98 (m, 1H), 3.98-3.91 (m, 1H) | 361 |
| | 4-phenyl-5-(5- | | | |
| | (trifluoromethyl)-1H- | | | |
| | benzo[d]imidazol-2- | | | |
| | yl)piperazin-2-one | | | |
| Example 370 Method 13 | 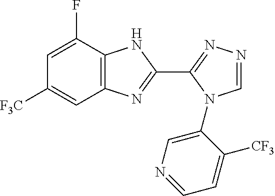 [SMILES: Fc1cc(C(F)(F)F)cc2nc(-c3nncn3-c3cnccc3C(F)(F)F)[nH]c12] | white solid, yield: 4% | ¹H NMR (400 MHz, Methanol-d₄) δ 9.17 (d, J = 5.1 Hz, 1H), 9.04 (s, 1H), 8.91 (s, 1H), 8.09 (d, J = 5.1 Hz, 1H), 7.65 (s, 1H), 7.42- 7.14 (m, 1H), 5.98 (d, J = 3.0 Hz, 1H). | 416 |
| | 7-fluoro-5- | | | |
| | (trifluoromethyl)-2-(4- | | | |
| | (4- | | | |
| | (trifluoromethyl)pyridin- | | | |
| | 3-yl)-4H-1,2,4-triazol- | | | |
| | 3-yl)-1H-indole | | | |
| Example 371 Method 10 | 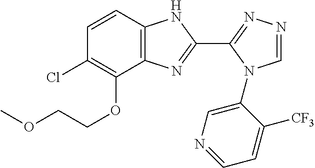 [SMILES: COCCOc1c(Cl)ccc2[nH]c(-c3nncn3-c3cnccc3C(F)(F)F)nc12] | white solid, yield: 47.3% | ¹H NMR (400 MHz, DMSO-d₆) δ 13.91 (br s, 1H), 9.15 (s, 1H), 9.14-9.07 (m, 2H), 8.10 (d, J = 5.2 Hz, 1H), 7.26 (d, J = 8.4 Hz, 1H), 7.11 (d, J = 8.4 Hz, 1H), 4.15-4.02 (m, 2H), 3.43-3.31 (m, 2H), 3.25 (s, 3H). | 439 |
| | 5-chloro-4-(2- | | | |
| | methoxyethoxy)-2-(4- | | | |
| | (4- | | | |
| | (trifluoromethyl)pyridin- | | | |
| | 3-yl)-4H-1,2,4-triazol- | | | |
| | 3-yl)-1H- | | | |
| | benzo[d]imidazole | | | |
| Example 372 Method 13 | 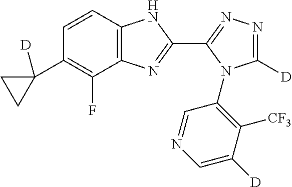 [SMILES: [2H]c1cncc(-n2c([2H])nnc2-c2nc3c(F)c(C4([2H])CC4)ccc3[nH]2)c1C(F)(F)F] | white solid, yield: 35.3% | ¹H NMR (400 MHz, Methanol-d₄) δ 9.07 (s, 1H), 8.97 (s, 1H), 7.17 (m, 1H), 6.88 (m, 1H), 1.04-0.89 (m, 2H), 0.78-0.64 (m, 2H). | 392 |
| | 5-(cyclopropyl-1-d)-4- | | | |
| | fluoro-2-(4-(4- | | | |
| | (trifluoromethyl)pyridin- | | | |
| | 3-yl-5-d)-4H-1,2,4- | | | |
| | triazol-3-yl-5-d)-1H- | | | |
| | benzo[d]imidazole | | | |
| Example 373 Method 19 | 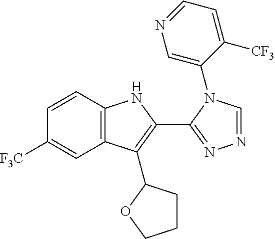 [SMILES: FC(F)(F)c1ccc2[nH]c(-c3nncn3-c3cnccc3C(F)(F)F)c(C3CCCO3)c2c1] | white solid, yield: 17% | ¹H NMR (500 MHz, CDCl₃) δ 8.90 (br s, 1H), 8.74 (br s, 1H), 8.21 (s, 1H), 8.10 (br d, J = 9.9 Hz, 1H), 7.70 (dd, J = 5.3, 7.4 Hz, 1H), 7.47 (d, J = 8.7 Hz, 1H), 6.92 (d, J = 8.5 Hz, 1H), 4.62- 4.49 (m, 1H), 4.34 (dt, J = 3.5, 8.5 Hz, 1H), 4.25-4.14 (m, 2H), 3.99-3.91 (m, 1H), 2.53-2.42 (m, 1H), 2.40-2.30 (m, 1H) | 468 |
| | 3-tetrahydrofuran-3-yl- | | | |
| | 5-(trifluoromethyl)-2- | | | |
| | [4-[4-(trifluoromethyl)- | | | |
| | 3-pyridyl]-1,2,4-triazol- | | | |
| | 3-yl]-1H-indole | | | |
| Example 374 Method 19 | 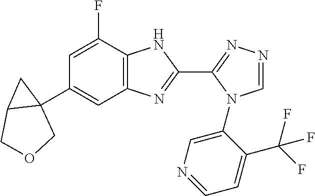 [SMILES: Fc1cc(C23COCC2C3)cc2nc(-c3nncn3-c3cnccc3C(F)(F)F)[nH]c12] | white solid, yield: 23% | ¹H NMR (400 MHz, Chloroform-d) δ 13.15 (br d, 1H, J = 3.1 Hz), 9.11 (d, 1H, J = 5.0 Hz), 8.80-8.90 (m, 1H), 8.40- 8.50 (m, 1H), 7.84 (d, 1H, J = 5.0 Hz), 7.67 (s, 1H), 6.74 (dd, 1H, J = 1.0, 11.5 Hz), 4.15 (d, 1H, J = 8.1 Hz), 3.80-4.10 (m, 3H), 1.89 (br t, 1H, J = 6.3 Hz), 1.00-1.10 (m, 2H). | 431 |
| Example 375 Method 19 | 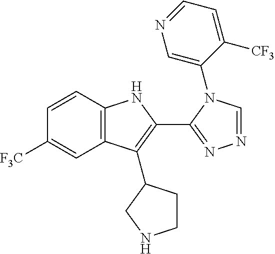 [SMILES: FC(F)(F)c1ccc2[nH]c(-c3nncn3-c3cnccc3C(F)(F)F)c(C3CCNC3)c2c1] | white solid, yield: 24% | ¹H NMR (400 MHz, MeOD) δ 9.07 (s, 1H), 9.05-9.01 (m, 2H), 8.73-8.45 (m, 1H), 8.13 (s, 1H), 7.92 (d, J = 5.2 Hz, 1H), 7.52- 7.47 (m, 2H), 4.07- 3.91 (m, 1H), 3.75- 3.53 (m, 3H), 3.46- 3.35 (m, 1H), 2.58- 2.43 (m, 1H), 2.38- 2.24 (m, 1H) | 467 |
| | 3-(pyrrolidin-3-yl)-5- | | | |
| | (trifluoromethyl)-2-(4-(4- | | | |
| | (trifluoromethyl)pyridin- | | | |
| | 3-yl)-4H-1,2,4-triazol-3- | | | |
| | yl)-1H-indole | | | |
| Example 376 Method 19 | 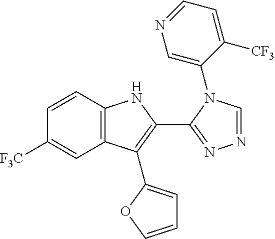 [SMILES: FC(F)(F)c1ccc2[nH]c(-c3nncn3-c3cnccc3C(F)(F)F)c(-c3ccco3)c2c1] | white solid, yield: 24% | ¹H NMR (400 MHz, MeOD) δ 8.95 (s, 1H), 8.80 (d, J = 5.1 Hz, 1H), 8.33 (s, 1H), 7.97 (s, 1H), 7.71 (d, J = 5.1 Hz, 1H), 7.66- 7.61 (m, 1H), 7.55 (d, J = 9.2 Hz, 2H), 6.52 (dd, J = 1.9, 3.3 Hz, 1H), 6.27 (d, J = 3.2 Hz, 1H) | 464 |
| | 3-(2-furyl)-5- | | | |
| | (trifluoromethyl)-2-[4- | | | |
| | [4-(trifluoromethyl)-3- | | | |
| | pyridyl]-1,2,4-triazol-3- | | | |
| | yl]-1H-indole | | | |
| Example 377 Method 10 | 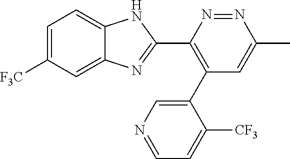 [SMILES: Cc1cc(-c2cnccc2C(F)(F)F)c(-c2nc3cc(C(F)(F)F)ccc3[nH]2)nn1] | White solid, | ¹H NMR (400 MHz, DMSO) δ 13.95 (d, J = 4.2 Hz, 1H), 8.99 (d, J = 5.2 Hz, 1H), 8.76 (d, J = 3.2 Hz, 1H), 7.92 (d, J = 5.3 Hz, 1H), 7.89 (s, 1H), 7.85-7.36 (m, 3H), 2.82 (s, 3H). | 424 |
| | 2-(6-Methyl-4-(4- | | | |
| | (trifluoromethyl)pyridin- | | | |
| | 3-yl)pyridazin-3-yl)-5- | | | |
| | (trifluoromethyl)-1H- | | | |
| | benzo[d]imidazole | | | |
| Example 378 Method 10 | 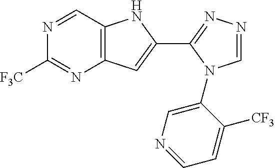 [SMILES: FC(F)(F)c1ncc2[nH]c(-c3nncn3-c3cnccc3C(F)(F)F)cc2n1] | White solid, yield: 4% | 1H NMR (400 MHz, DMSO-d₆) δ 13.48 (s, 1H), 9.21 (m, 2H), 9.17 (s, 1H), 9.09 (s, 1H), 8.18 (d, J = 8.2 Hz, 1H), 5.93 (s, 1H). | 400 |
| | 2-(trifluoromethyl)-6- | | | |
| | (4-(4- | | | |
| | (trifluoromethyl)pyridin- | | | |
| | 3-yl)-4H-1,2,4-triazol- | | | |
| | 3-yl)-5H-pyrrolo[3,2- | | | |
| | d]pyrimidine | | | |
| Example 379 Method 19 | 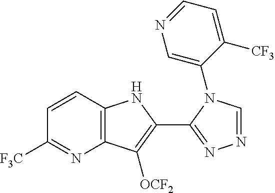 | White solid | ¹H NMR (400 MHz, Methanol-d₄) δ 9.02 (d, J = 5.1 Hz, 1H), 8.96 (s, 1H), 8.89 (s, 1H), 8.05 (d, J = 8.7 Hz, 1H), 7.95 (d, J = 5.2 Hz, 1H), 7.69 (d, J = 8.7 Hz, 1H), 7.47 (t, J = 75.0 Hz, 2H). | 465 |
| | 3-(difluoromethoxy)-5- | | | |
| | (trifluoromethyl)-2-(4- | | | |
| | (4- | | | |
| | (trifluoromethyl)pyridin- | | | |
| | 3-yl)-4H-1,2,4-triazol- | | | |
| | 3-yl)-1H-pyrrolo[2,3- | | | |
| | b]pyridine | | | |
| Example 380 Method 14 | 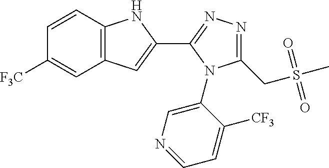 [SMILES: CS(=O)(=O)Cc1nnc(-c2cc3cc(C(F)(F)F)ccc3[nH]2)n1-c1cnccc1C(F)(F)F] | white solid, yield: 45.33% | ¹H NMR (400 MHz, DMSO-d₆) δ 12.58 (s, 1H), 9.25 (d, J = 5.1 Hz, 1H), 9.05 (s, 1H), 8.17 (d, J = 5.2 Hz, 1H), 7.89 (s, 1H), 7.60 (d, J = 8.7 Hz, 1H), 7.45 (dd, J = 8.7, 1.8 Hz, 1H), 5.66 (s, 1H), 5.09 (d, J = 15.1 Hz, 1H), 4.70 (d, J = | 490 |
| | 2-(5- | | 15.1 Hz, 1H), 3.18 (s, | |
| | ((methylsulfonyl)methyl)- | | 3H). | |
| | 4-(4- | | | |
| | (trifluoromethyl)pyridin- | | | |
| | 3-yl)-4H-1,2,4-triazol- | | | |
| | 3-yl)-5- | | | |
| | (trifluoromethyl)-1H- | | | |
| | indole | | | |
| Example 381 Method 14 | 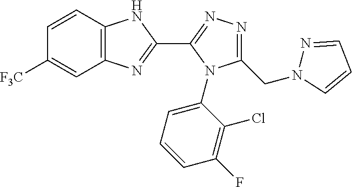 [SMILES: Fc1cccc(-n2c(Cn3cccn3)nnc2-c2nc3cc(C(F)(F)F)ccc3[nH]2)c1Cl] | white solid, yield: 26.55% | ¹H NMR (400 MHz, Methanol-d₄) δ 7.89- 7.47 (m, 5H), 7.39 (d, J = 1.9 Hz, 1H), 7.35- 7.26 (m, 2H), 6.18 (t, J = 2.2 Hz, 1H), 5.67- 5.48 (m, 2H). | 462 |
| | 2-(5-((1H-pyrazol-1- | | | |
| | yl)methyl)-4-(2-chloro- | | | |
| | 3-fluorophenyl)-4H- | | | |
| | 1,2,4-triazol-3-yl)-5- | | | |
| | (trifluoromethyl)-1H- | | | |
| | benzo[d]imidazole | | | |
| Example 382 Method 14 | 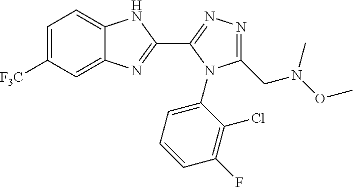 [SMILES: CON(C)Cc1nnc(-c2nc3cc(C(F)(F)F)ccc3[nH]2)n1-c1cccc(F)c1Cl] | white solid, yield: 21.66% | ¹H NMR (400 MHz, Methanol-d₄) δ 7.85- 7.78 (m, 1H), 7.71- 7.65 (m, 1H), 7.63- 7.48 (m, 4H), 4.08- 3.80 (m, 2H), 3.15 (s, 3H), 2.52 (s, 3H). | 455 |
| | N-((4-(2-chloro-3- | | | |
| | fluorophenyl)-5-(5- | | | |
| | (trifluoromethyl)-1H- | | | |
| | benzo[d]imidazol-2-yl)- | | | |
| | 4H-1,2,4-triazol-3- | | | |
| | yl)methyl)-N,O- | | | |
| | dimethylhydroxylamine | | | |
| Example 383 Method 14 | 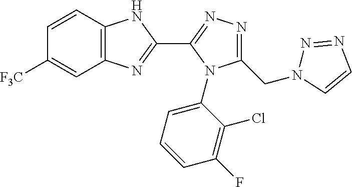 [SMILES: Fc1cccc(-n2c(Cn3ccnn3)nnc2-c2nc3cc(C(F)(F)F)ccc3[nH]2)c1Cl] | white solid, yield: 32.65% | ¹H NMR (400 MHz, DMSO-d₆) δ 14.12 (s, 1H), 7.80 (s, 1H), 7.72- 7.65 (m, 4H), 7.59 (q, J = 3.4 Hz, 2H), 7.56- 7.50 (m, 1H), 5.89 (d, J = 1.9 Hz, 2H). | 463 |
| | 2-(5-((1H-1,2,3-triazol- | | | |
| | 1-yl)methyl)-4-(2- | | | |
| | chloro-3-fluorophenyl)- | | | |
| | 4H-1,2,4-triazol-3-yl)- | | | |
| | 5-(trifluoromethyl)-1H- | | | |
| | benzo[d]imidazole | | | |
| Example 384 Method 14 | 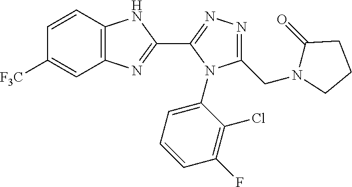 [SMILES: O=C1CCCN1Cc1nnc(-c2nc3cc(C(F)(F)F)ccc3[nH]2)n1-c1cccc(F)c1Cl] | white solid, yield: 30.25% | ¹H NMR (400 MHz, DMSO-d₆) δ 7.83- 7.79 (m, 1H), 7.75- 7.67 (m, 3H), 7.65- 7.51 (m, 2H), 4.65- 4.45 (m, 2H), 3.36- 3.20 (m, 2H), 2.06 (t, J = 8.1 Hz, 2H), 1.94- 1.79 (m, 2H). | 479 |
| | 1-((4-(2-chloro-3- | | | |
| | fluorophenyl)-5-(5- | | | |
| | (trifluoromethyl)-1H- | | | |
| | benzo[d]imidazol-2-yl)- | | | |
| | 4H-1,2,4-triazol-3- | | | |
| | yl)methyl)pyrrolidin-2- | | | |
| | one | | | |
| Example 385 Method 18 | 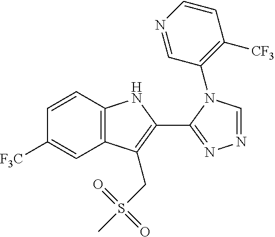 [SMILES: CS(=O)(=O)Cc1c(-c2nncn2-c2cnccc2C(F)(F)F)[nH]c2ccc(C(F)(F)F)cc12] | white solid, yield: 34% | ¹H NMR (400 MHz, MeOD) δ 8.99 (d, J = 5.1 Hz, 1H), 8.97 (s, 1H), 8.88 (s, 1H), 8.24 (s, 1H), 8.02 (d, J = 5.1 Hz, 1H), 7.54-7.40 (m, 2H), 5.05-4.95 (m, 2H), 3.03 (s, 3H) | 490 |
| | 3- | | | |
| | (methylsulfonylmethyl)- | | | |
| | 5-(trifluoromethyl)-2- | | | |
| | [4-[4-(trifluoromethyl)- | | | |
| | 3-pyridyl]-1,2,4-triazol- | | | |
| | 3-yl]-1H-indole | | | |
| Example 386 Method 18 | 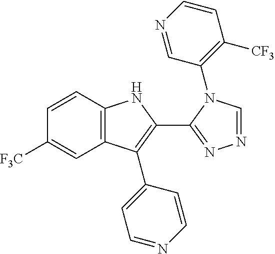 [SMILES: FC(F)(F)c1ccc2[nH]c(-c3nncn3-c3cnccc3C(F)(F)F)c(-c3ccncc3)c2c1] | white solid, yield: 13% | ¹H NMR (500 MHz, MeOD) δ 8.90 (s, 1H), 8.76 (d, J = 5.0 Hz, 1H), 8.48 (d, J = 5.3 Hz, 2H), 8.09 (s, 1H), 7.83 (s, 1H), 7.70 (d, J = 8.7 Hz, 1H), 7.64 (d, J = 5.2 Hz, 1H), 7.58 (d, J = 8.5 Hz, 1H), 7.09 (d, J = 5.6 Hz, 2H) | 475 |
| | 3-(4-pyridyl)-5- | | | |
| | (trifluoromethyl)-2-[4- | | | |
| | [4-(trifluoromethyl)-3- | | | |
| | pyridyl]-1,2,4-triazol-3- | | | |
| | yl]-1H-indole | | | |
| Example 387 Method 18 | 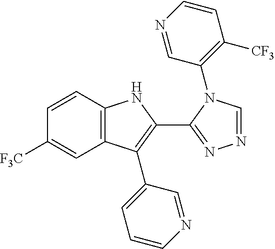 [SMILES: FC(F)(F)c1ccc2[nH]c(-c3nncn3-c3cnccc3C(F)(F)F)c(-c3cccnc3)c2c1] | white solid, yield: 23% | ¹H NMR (400 MHz, Methanol-d₄) δ 8.91 (s, 1H), 8.83 (d, J = 5.2 Hz, 1H), 8.71 (d, J = 5.4 Hz, 1H), 8.58 (s, 1H), 8.32 (s, 1H), 8.08 (dd, J = 1.6, 8.0 Hz, 1H), 7.84 (s, 1H), 7.83- 7.77 (m, 1H), 7.74 (d, J = 5.2 Hz, 1H), 7.70- 7.66 (m, 1H), 7.62- 7.57 (m, 1H) | 475 |
| | 3-(pyridin-3-yl)-5- | | | |
| | (trifluoromethyl)-2-(4- | | | |
| | (4- | | | |
| | (trifluoromethyl)pyridin- | | | |
| | 3-yl)-4H-1,2,4-triazol- | | | |
| | 3-yl)-1H-indole | | | |
| Example 388 Method 14 | 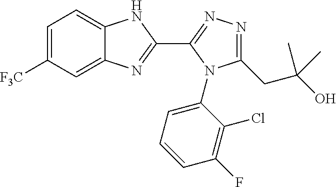 [SMILES: CC(C)(O)Cc1nnc(-c2nc3cc(C(F)(F)F)ccc3[nH]2)n1-c1cccc(F)c1Cl] | white solid, yield: 26.33% | ¹H NMR (400 MHz, DMSO-d₆) δ 7.82- 7.69 (m, 2H), 7.69- 7.61 (m, 3H), 7.51 (d, J = 8.5 Hz, 1H), 2.79- 2.60 (m, 2H), 1.24 (s, 3H), 1.17 (s, 3H). | 454 |
| | 1-(4-(2-chloro-3- | | | |
| | fluorophenyl)-5-(5- | | | |
| | (trifluoromethyl)-1H- | | | |
| | benzo[d]imidazol-2-yl)- | | | |
| | 4H-1,2,4-triazol-3-yl)- | | | |
| | 2-methylpropan-2-ol | | | |
| Example 389 Method 10 | 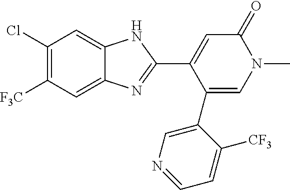 [SMILES: Cn1cc(-c2cnccc2C(F)(F)F)c(-c2nc3cc(C(F)(F)F)c(Cl)cc3[nH]2)cc1=O] | White solid, yield: 20% | ¹H NMR (400 MHz, Methanol-d₄) δ 8.82 (d, J = 5.2 Hz, 1H), 8.73 (s, 1H), 7.92 (s, 1H), 7.85- 7.74 (m, 2H), 7.72 (d, J = 5.2 Hz, 1H), 7.12 (s, 1H), 3.71 (s, 3H). | 473 |
| | Preparation of 4-(5- | | | |
| | chloro-6- | | | |
| | (trifluoromethyl)-1H- | | | |
| | benzo[d]imidazol-2-yl)- | | | |
| | 1-methyl-4′- | | | |
| | (trifluoromethyl)-[3,3′- | | | |
| | bipyridin]-6(1H)-one | | | |
| Example 390 Method 18 | 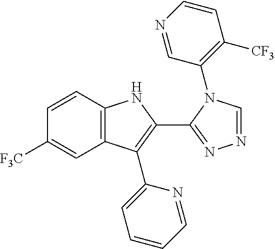 [SMILES: FC(F)(F)c1ccc2[nH]c(-c3nncn3-c3cnccc3C(F)(F)F)c(-c3ccccn3)c2c1] | White solid | ¹H NMR (400 MHz, METHANOL-d₄) δ 8.91 (s, 1H), 8.79 (d, J = 5.0 Hz, 1H), 8.61 (d, J = 5.1 Hz, 1H), 8.20 (s, 1H), 8.11-8.06 (m, 1H), 8.00 (s, 1H), 7.73- 7.68 (m, 2H), 7.63- 7.58 (m, 2H), 7.47 (d, J = 8.0 Hz, 1H) | 475 |
| | 3-(pyridin-2-yl)-5- | | | |
| | (trifluoromethyl)-2-(4- | | | |
| | (4- | | | |
| | (trifluoromethyl)pyridin- | | | |
| | 3-yl)-4H-1,2,4-triazol- | | | |
| | 3-yl)-1H-indole | | | |
| Example 391 Method 13 | 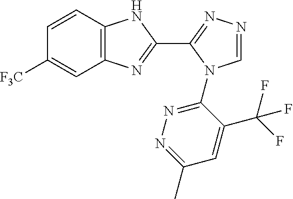 [SMILES: Cc1cc(C(F)(F)F)c(-n2cnnc2-c2nc3cc(C(F)(F)F)ccc3[nH]2)nn1] | white solid, yield: 3.3% | ¹H NMR (400 MHz, DMSO-d₆) δ 9.17 (s, 1H), 8.49 (s, 1H), 8.41 (s, 1H), 7.67 (s, 1H), 7.54 (s, 1H), 7.35 (s, 1H), 2.94 (s, 3H). | 414 |
| | 2-(4-(6-methyl-4- | | | |
| | (trifluoromethyl)pyridazin- | | | |
| | 3-yl)-4H-1,2,4- | | | |
| | triazol-3-yl)-5- | | | |
| | (trifluoromethyl)-1H- | | | |
| | benzo[d]imidazole | | | |
| Example 392 Method 8 | 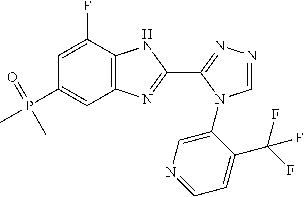 [SMILES: CP(C)(=O)c1cc(F)c2[nH]c(-c3nncn3-c3cnccc3C(F)(F)F)nc2c1] | white solid, yield: 46% | ¹H NMR (400 MHz, MeOD) δ 9.09 (d, J = 5.0 Hz, 1H), 8.99 (s, 2H), 8.00 (d, J = 5.1 Hz, 1H), 7.81 (d, J = 12.6 Hz, 1H), 7.36 (t, J = 11.1 Hz, 1H), 1.81 (d, J = 13.5 Hz, 6H) | 425 |
| | 5-dimethylphosphoryl- | | | |
| | 7-fluoro-2-[4-[4- | | | |
| | (trifluoromethyl)-3- | | | |
| | pyridyl]-1,2,4-triazol-3- | | | |
| | yl]-1H-benzimidazole | | | |
| Example 393 Method 4 | 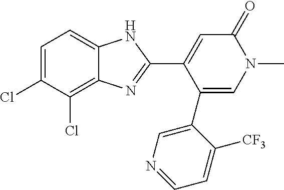 [SMILES: Cn1cc(-c2cnccc2C(F)(F)F)c(-c2nc3c(Cl)c(Cl)ccc3[nH]2)cc1=O] | white solid, yield: 31% | ¹H NMR (400 MHz, MeOD) δ 8.84 (d, J = 5.4 Hz, 1H), 8.80 (s, 1H), 7.91 (s, 1H), 7.78 (d, J = 5.4 Hz, 1H), 7.40-7.29 (m, 2H), 7.13 (s, 1H), 3.69 (s, 3H). | 439 |
| | 4-(4,5-dichloro-1H- | | | |
| | benzo[d]imidazol-2-yl)- | | | |
| | 1-methyl-4′- | | | |
| | (trifluoromethyl)-[3,3′- | | | |
| | bipyridin]-6(1H)-one | | | |
| Example 394 Method 13 | 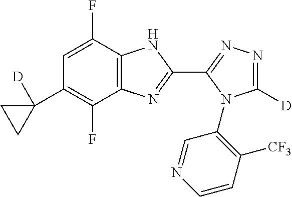 [SMILES: [2H]c1nnc(-c2nc3c(F)c(C4([2H])CC4)cc(F)c3[nH]2)n1-c1cnccc1C(F)(F)F] | white solid, | ¹H NMR (400 MHz, Methanol-d₄) δ 9.06 (d, J = 4.9 Hz, 1H), 8.96 (s, 1H), 7.98 (d, J = 5.2 Hz, 1H), 6.70-6.40 (m, 1H), 1.10-0.93 (m, 2H), 0.79-0.64 (m, 2H). | 409 |
| | 5-(cyclopropyl-1-d)- | | | |
| | 4,7-difluoro-2-(4-(4- | | | |
| | (trifluoromethyl)pyridin- | | | |
| | 3-yl)-4H-1,2,4-triazol- | | | |
| | 3-yl-5-d)-1H- | | | |
| | benzo[d]imidazole | | | |
| Example 395 Method 4 | 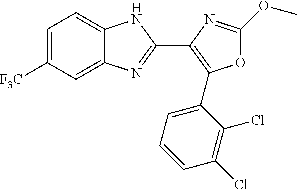 [SMILES: COc1nc(-c2nc3cc(C(F)(F)F)ccc3[nH]2)c(-c2cccc(Cl)c2Cl)o1] | white solid, yield: 10.4% | ¹H NMR (400 MHz, Methanol-d₄) δ 7.85 (s, 1H), 7.70 (dd, J = 8.0, 1.6 Hz, 1H), 7.67 (s, 1H), 7.59 (dd, J = 8.0, 1.6 Hz, 1H), 7.53 (d, J = 8.4 Hz, 1H), 7.45 (t, J = 8.0 Hz, 1H), 4.26 (s, 3H). | 427 |
| | 4-(5-chloro-6- | | | |
| | (trifluoromethyl)-1H- | | | |
| | benzo[d]imidazol-2-yl)- | | | |
| | 1-methyl-4′- | | | |
| | (trifluoromethyl)-[3,3′- | | | |
| | bipyridin]-6(1H)-one | | | |
| Example 396 Method 14 | 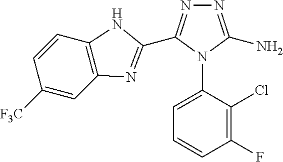 [SMILES: Nc1nnc(-c2nc3cc(C(F)(F)F)ccc3[nH]2)n1-c1cccc(F)c1Cl] | white solid, yield: 86.7% | ¹H NMR (400 MHz, DMSO-d₆) δ 7.79 (s, 1H), 7.67-7.47 (m, 5H). | 397 |
| | 4-(2-chloro-3- | | | |
| | fluorophenyl)-5-(5- | | | |
| | (trifluoromethyl)-1H- | | | |
| | benzo[d]imidazol-2-yl)- | | | |
| | 4H-1,2,4-triazol-3- | | | |
| | amine | | | |
| Example 397 Method 10 | 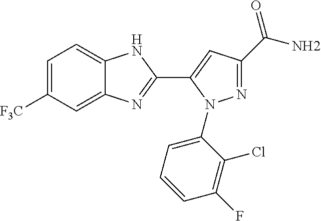 [SMILES: NC(=O)c1cc(-c2nc3cc(C(F)(F)F)ccc3[nH]2)n(-c2cccc(F)c2Cl)n1] | white solid, yield: 25.1% | ¹H NMR (400 MHz, DMSO-d₆) δ 7.88 (s, 1H), 7.75 (m, 1H), 7.72- 7.40 (m, 5H). | 424 |
| | 1-(2-chloro-3- | | | |
| | fluorophenyl)-5-(5- | | | |
| | (trifluoromethyl)-1H- | | | |
| | benzo[d]imidazol-2-yl)- | | | |
| | 1H-pyrazole-3- | | | |
| | carboxamide | | | |
| Example 398 Method 14 | 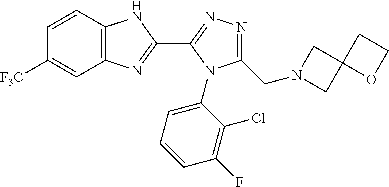 [SMILES: Fc1cccc(-n2c(CN3CC4(CCO4)C3)nnc2-c2nc3cc(C(F)(F)F)ccc3[nH]2)c1Cl] | white solid, yield: 26.2% | ¹H NMR (400 MHz, Chloroform-d) δ 13.28 (br s, 1H), 8.52-7.80 (m, 2H), 7.60 (m, 1H), 7.54-7.44 (m, 2H), 7.38 (m, 1H), 4.50 (t, J = 7.6 Hz, 2H), 3.81- 3.63 (m, 4H), 3.38 (s, 2H), 2.82 (t, J = 7.6 Hz, 2H). | 493 |
| | 6-((4-(2-chloro-3- | | | |
| | fluorophenyl)-5-(6- | | | |
| | (trifluoromethyl)-1H- | | | |
| | benzo[d]imidazol-2-yl)- | | | |
| | 4H-1,2,4-triazol-3- | | | |
| | yl)methyl)-1-oxa-6- | | | |
| | azaspiro[3.3]heptane | | | |
| Example 399 Method 10 | 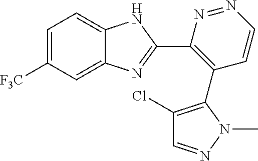 [SMILES: Cn1ncc(Cl)c1-c1ccnnc1-c1nc2cc(C(F)(F)F)ccc2[nH]1] | white solid, yield: 49% | ¹H NMR (400 MHz, Methanol-d₄) δ 9.47 (d, J = 5.3 Hz, 1H), 7.98- 7.83 (m, 2H), 7.80- 7.68 (m, 1H), 7.63- 7.48 (m, 2H), 3.67 (s, 3H) | 393 |
| | 2-(4-(4-chloro-1- | | | |
| | methyl-1H-pyrazol-5- | | | |
| | yl)pyridazin-3-yl)-5- | | | |
| | (trifluoromethyl)-1H- | | | |
| | benzo[d]imidazole | | | |
| Example 400 Method 17 | 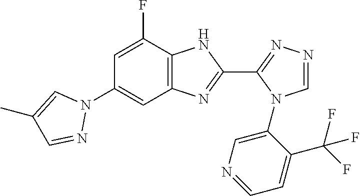 [SMILES: Cc1cnn(-c2cc(F)c3[nH]c(-c4nncn4-c4cnccc4C(F)(F)F)nc3c2)c1] | white solid, yield: 28% | ¹H NMR (400 MHz, Methanol-d₄) δ 9.08 (d, J = 5.2 Hz, 1H), 8.98 (d, J = 8.0 Hz, 2H), 8.09 (s, 1H), 8.00 (d, J = 5.2 Hz, 1H), 7.65 (s, 1H), 7.38 (s, 1H), 6.32 (d, J = 2.4 Hz, 1H), 2.34 (s, 3H). | 429 |
| | 7-fluoro-5-(4-methyl- | | | |
| | 1H-pyrazol-1-yl)-2-(4- | | | |
| | (4- | | | |
| | (trifluoromethyl)pyridin- | | | |
| | 3-yl)-4H-1,2,4-triazol- | | | |
| | 3-yl)-1H- | | | |
| | benzo[d]imidazole | | | |

### Preparation of 2-(4-(2-chloro-3-fluorophenyl)-4H-1,2,4-triazol-3-yl)-5-ethyl-1,5-dihydro-4H-pyrrolo[3,2-c]pyridin-4-one (308)

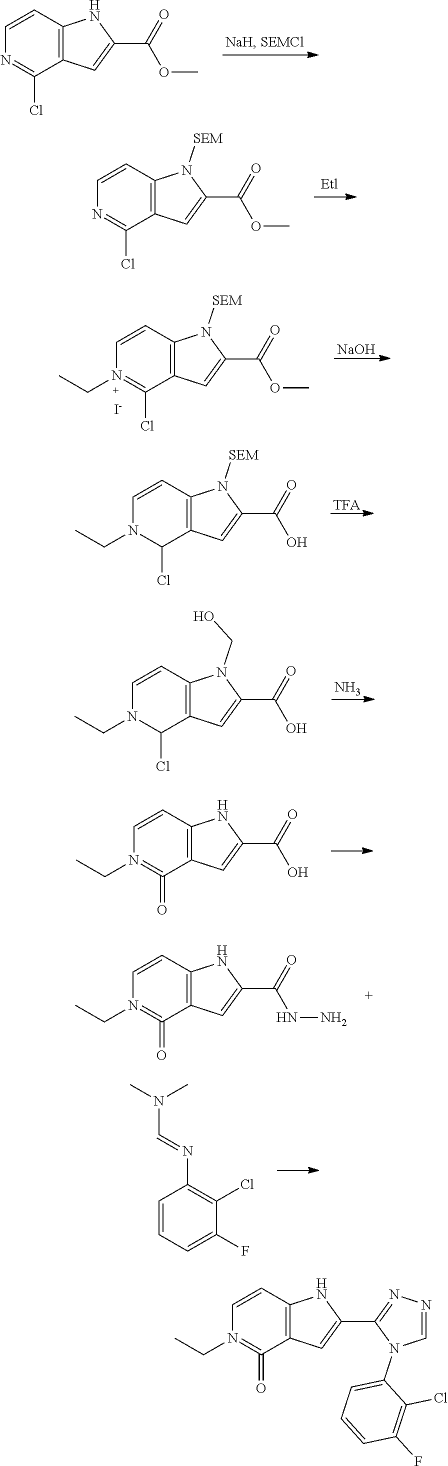

### Step 1

To a solution of NaH (1.30 eq, 306 mg, 7.65 mmol) in THF (30 mL) was added methyl 4-chloro-1H-pyrrolo[3,2-c]pyridine-2-carboxylate (1.00 eq, 1239 mg, 5.88 mmol) at 0° C. The mixture was stirred for 30 min. SEMCl (1.30 eq, 1.4 mL, 7.65 mmol) was added. The mixture was warmed to rt and stirred overnight. The reaction was quenched with sat. aq. NH₄Cl, extracted with EA. The organic phase was dried with anhydrous Na₂SO₄ and concentrated and the residue was purified with FCC to give methyl 4-chloro-1-(2-trimethylsilylethoxymethyl)pyrrolo[3,2-c]pyridine-2-carboxylate (1250 mg, 3.67 mmol, 62.34% yield).

### Step 2

methyl 4-chloro-1-(2-trimethylsilylethoxymethyl)pyrrolo[3,2-c]pyridine-2-carboxylate (1.00 eq, 969 mg, 2.84 mmol) was dissolved in iodoethane (10.0 eq, 2.3 mL, 28.4 mmol). The mixture was heated to 80° C. and stirred overnight. The mixture was concentrated under reduced pressure. The residue was used in the next step directly.

### Step 3

To a solution of methyl 4-chloro-5-ethyl-1-(2-trimethylsilylethoxymethyl)pyrrolo[3,2-c]pyridin-5-ium-2-carboxylate iodide (1.00 eq, 1391 mg, 2.80 mmol) in 1,4-Dioxane (15 mL) and water (15 mL) was added NaOH (10.0 eq, 1120 mg, 28.0 mmol). The mixture was stirred for 2 h at rt. The mixture was acidified with 4N HCl, and extracted with EA. The organic phase was dried with anhydrous Na₂SO₄ and concentrated under reduced pressure. The residue was purified with FCC to give 5-ethyl-4-oxo-1-(2-trimethylsilylethoxymethyl)pyrrolo[3,2-c]pyridine-2-carboxylic acid (813 mg, 2.42 mmol, 86.30% yield).

### Step 4

To a solution of 5-ethyl-4-oxo-1-(2-trimethylsilylethoxymethyl)pyrrolo[3,2-c]pyridine-2-carboxylic acid (1.00 eq, 1360 mg, 4.04 mmol) in DCM (20 mL) was added TFA (64.6 eq, 20 mL, 261 mmol). The mixture was stirred for 4 h at rt, and then concentrated, and the residue was used in next step directly.

### Step 5

To a solution of 5-ethyl-1-(hydroxymethyl)-4-oxo-pyrrolo[3,2-c]pyridine-2-carboxylic acid (1.00 eq, 945 mg, 4.00 mmol) in THF (20 mL) was added NH₃ in water (10 mL). The mixture was stirred overnight at rt and then concentrated under reduced pressure. Water was added. Aq. HCl (1N) was added until pH=1. The mixture was extracted with EA, and dried with anhydrous Na₂SO₄. The organic phase was removed under reduced pressure. The residue was purified with FCC to give 5-ethyl-4-oxo-1H-pyrrolo[3,2-c]pyridine-2-carboxylic acid (531 mg, 2.58 mmol, 64.38% yield).

### Step 6

To a solution of 5-ethyl-4-oxo-1H-pyrrolo[3,2-c]pyridine-2-carboxylic acid (1.00 eq, 531 mg, 2.58 mmol) in THF (15 mL) was added N-methyl-morpholine (3.00 eq, 0.85 mL, 7.73 mmol) followed by addition of isobutyl chloroformate (1.20 eq, 0.40 mL, 3.09 mmol) at 0° C. The mixture was stirred for 20 min and N₂H₄·H₂O (5.00 eq, 805 mg, 12.9 mmol) was added dropwise. The mixture was warmed to rt and stirred for 1.5 h. The mixture was extracted with EA, dried with anhydrous Na₂SO₄, and concentrated under reduced pressure. The residue was purified with FCC to give 5-ethyl-4-oxo-1H-pyrrolo[3,2-c]pyridine-2-carbohydrazide (423 mg, 1.92 mmol, 74.59% yield).

### Step 7

To a solution of N′-(2-chloro-3-fluoro-phenyl)-N,N-dimethyl-formamidine (2.00 eq, 164 mg, 0.817 mmol) in MeCN (3 mL) and acetic acid (1 mL) was added 5-ethyl-4-oxo-1H-pyrrolo[3,2-c]pyridine-2-carbohydrazide (1.00 eq, 90 mg, 0.409 mmol). The mixture was heated to 90° C. and stirred for 2 h. The mixture was concentrated under reduced pressure and the residue was purified with pre-HPLC to give 2-[4-(2-chloro-3-fluoro-phenyl)-1,2,4-triazol-3-yl]-5-ethyl-1H-pyrrolo[3,2-c]pyridin-4-one (2.0 mg, 0.00559 mmol, 1.37% yield) as a white solid. MS (ESI) m/z 358 [M+H]⁺. ¹H NMR (400 MHz, MeOD) δ 8.73 (s, 1H), 7.69-7.62 (m, 2H), 7.59-7.55 (m, 1H), 7.37 (d, J=7.3 Hz, 1H), 6.64 (d, J=7.3 Hz, 1H), 6.15 (s, 1H), 4.03 (q, J=7.2 Hz, 2H), 1.29 (t, J=7.1 Hz, 4H).

### Preparation of 2-(5-(2,3-dichlorophenyl)-1H-1,2,3-triazol-1-yl)-5-(trifluoromethyl)-1H-benzo[d]imidazole (315)

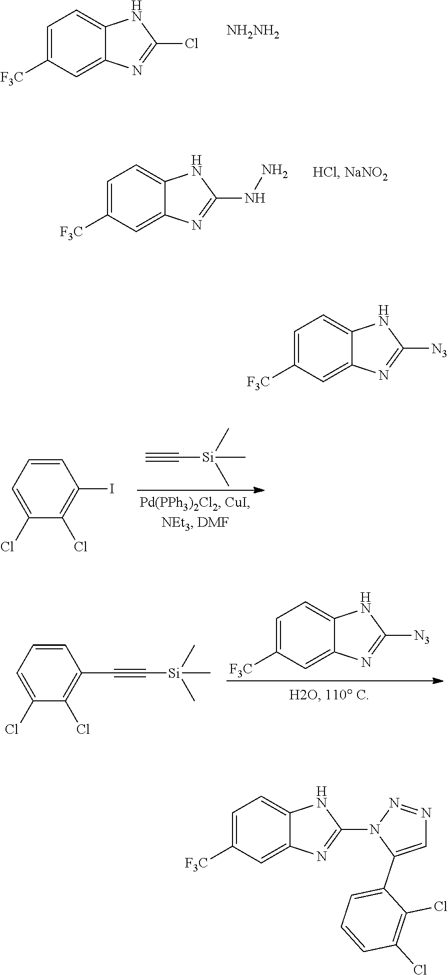

### Step 1. 2-hydrazineyl-5-(trifluoromethyl)-1H-benzoimidazole

To a solution of 2-chloro-5-(trifluoromethyl)-1H-benzo[d]imidazole (1 g, 4.54 mmol) in THF (10 mL) was added hydrazine (80%, 2 mL). The reaction was heated to 80° C. and stirred overnight. The resulting mixture was diluted with EA and water. The organic layer was washed with brine, dried over Na₂SO₄ and concentrated to give the crude product (1 g, 100% yield) as a yellow oil. MS (ESI) m/z 2137[M+H]⁺.

### Step 2. 2-azido-5-(trifluoromethyl)-1H-benzo[d]imidazole

To the mixture of 2-hydrazineyl-5-(trifluoromethyl)-1H-benzo[d]imidazole (300 mg, 1.39 mmol) in aqueous HCl (3 N, 5 mL) at an ice bath was added aqueous NaN₂ (200 mg, 2.90 mmol) dropwise. The reaction was stirred for 2 hours. The resulting mixture was diluted with water and EA. The organic layer was washed with brine, dried over Na₂SO₄ and concentrated to give a crude product (0.3 g, 100% yield) as a yellow oil. MS (ESI) m/z 359 [M+H]⁺.

### Step 3. ((2,3-dichlorophenyl)ethynyl)trimethylsilane

A mixture of 1,2-dichloro-3-iodobenzene (1.0 g, 3.68 mmol), ethynyltrimethylsilane (3 mL), triethylamine (3 mL), Pd(PPh₃)₂Cl₂ (100 mg, 0.14 mmol) and CuI (30 mg, 0.16 mmol) in DMF (5 mL) was heated to 100-C and stirred overnight at N₂ atmosphere. The resulting mixture was diluted with water and EA. The organic layer was washed with brine, dried over Na₂SO₄ and concentrated. The residue was purified by flash to give the product (0.8 g, 9000 yield) as a yellow oil.

### Step 4. 2-(5-(2,3-dichlorophenyl)-1H-1,2,3-triazol-1-yl)-5-(trifluoromethyl)-1H-benzo[d]imidazole

A mixture of 2-hydrazineyl-5-(trifluoromethyl)-1H-benzo[d]imidazole (300 mg, 1.24 mmol) and 2-azido-5-(trifluoromethyl)-1H-benzo[d]imidazole (280 mg, 1.23 mmol) in water (5 mL) was heated to reflux overnight. The resulting mixture was diluted with water and EA. The organic layer was concentrated and the residue was purified through pre-HPLC to give the product (3 mg, 6.1% yield) as a white solid. MS (ESI) m/z 398 [M+H]. ¹H NMR (400 MHz, d6-DMSO) δ 14.15 (bs, 1H), 8.26 (s, 1H), 7.85 (s, 1H), 7.81 (d, J=8.0 Hz, 1H), 7.70 (d, J=8.0 Hz, 1H), 7.60-7.47 (m, 3H).

### Preparation of 2-(1-(7-fluoro-2-(4-(4-(trifluoromethyl)pyridin-3-yl)-4H-1,2,4-triazol-3-yl)-1H-benzo[d]imidazol-5-yl)ethyl)thiazole (412) Method 20

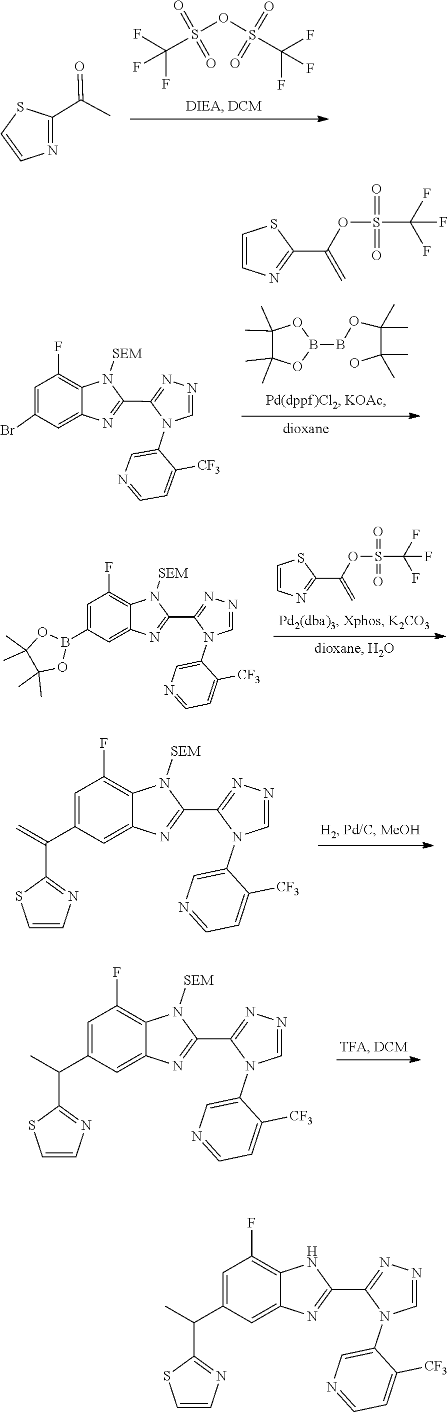

### Step 1. 1-(thiazol-2-yl)vinyl trifluoromethanesulfonate

To a solution of 1-thiazol-2-ylethanone (1.00 eq, 1000 mg, 7.86 mmol) in DCM (20 mL) were added DIEA (3.00 eq, 4.1 mL, 23.6 mmol) and trifluoromethanesulfonic anhydride (2.00 eq, 4438 mg, 15.7 mmol) at −30° C. The mixture was stirred for 1 hour at −30° C. under inert atmosphere. The mixture was diluted with EtOAc, and washed with water. The organic layer was separated, dried over anhydrous sodium sulfate and concentrated under vacuum. The residue was purified by silica gel flash column chromatography (PE/EtOAc=1/9) to give 1-thiazol-2-ylvinyl trifluoromethanesulfonate (1000 mg, 3.47 mmol, 44.15%% yield) as brown oil. MS (ESI) m/z 260 [M+H]⁺.

### Step 2. 7-fluoro-5-(4,4,5,5-tetramethyl-1,3,2-dioxaborolan-2-yl)-2-(4-(4-(trifluoromethyl)pyridin-3-yl)-4H-1,2,4-triazol-3-yl)-1-((2-(trimethylsilyl)ethoxy)methyl)-1H-benzo[d]imidazole

To a mixture of 5-bromo-7-fluoro-2-(4-(4-(trifluoromethyl)pyridin-3-yl)-4H-1,2,4-triazol-3-yl)-1-((2-(trimethylsilyl)ethoxy)methyl)-1H-benzo[d]imidazole (1.00 eq, 2000 mg, 3.59 mmol) and 4,4,4′,4′,5,5,5′,5′-octamethyl-2,2′-bi(1,3,2-dioxaborolane) (2.00 eq, 1822 mg, 7.18 mmol) in 1,4-Dioxane (20 mL) were added Pd(dppf)Cl₂ (0.100 eq, 293 mg, 0.359 mmol) and potassium acetate (3.00 eq, 1055 mg, 10.8 mmol). The reaction mixture was stirred for 1 hour at 100° C. under inert atmosphere. The mixture was diluted with EtOAc, and washed with water. The organic layer was separated, dried over anhydrous sodium sulfate and concentrated under vacuum. The crude product was purified by prep-HPLC (H2O:ACN=30:70~100:0; collect products at 90% ACN) to give 7-fluoro-5-(4,4,5,5-tetramethyl-1,3,2-dioxaborolan-2-yl)-2-(4-(4-(trifluoromethyl)pyridin-3-yl)-4H-1,2,4-triazol-3-yl)-1-((2-(trimethylsilyl)ethoxy)methyl)-1H-benzo[d]imidazole (2000 mg, 2.98 mmol, 82.99%% yield) as a brown oil. MS (ESI) m/z 605 [M+H]⁺.

### Step 3. 2-(1-(7-fluoro-2-(4-(4-(trifluoromethyl)pyridin-3-yl)-4H-1,2,4-triazol-3-yl)-1-((2-(trimethylsilyl)ethoxy)methyl)-1H-benzo[d]imidazol-5-yl)vinyl)thiazole

To a mixture of 7-fluoro-5-(4,4,5,5-tetramethyl-1,3,2-dioxaborolan-2-yl)-2-(4-(4-(trifluoromethyl)pyridin-3-yl)-4H-1,2,4-triazol-3-yl)-1-((2-(trimethylsilyl)ethoxy)methyl)-1H-benzo[d]imidazole (1.00 eq, 300 mg, 0.496 mmol) and 1-(thiazol-2-yl)vinyl trifluoromethanesulfonate (2.00 eq, 257 mg, 0.993 mmol) in 1,4-Dioxane (15 mL) and water (1 mL) were added Pd₂(dba)₃ (0.200 eq, 91 mg, 0.0993 mmol) and K₂CO₃ (3.00 eq, 191 mg, 1.49 mmol). The reaction mixture was stirred for 1 hour at 100° C. under inert atmosphere. The mixture was diluted with EtOAc, and washed with water. The organic layer was separated, dried over anhydrous sodium sulfate and concentrated under vacuum. The crude product was purified by prep-HPLC (H2O:ACN=30:70~100:0; collect products at 90% ACN) to give 2-(1-(7-fluoro-2-(4-(4-(trifluoromethyl)pyridin-3-yl)-4H-1,2,4-triazol-3-yl)-1-((2-(trimethylsilyl)ethoxy)methyl)-1H-benzo[d]imidazol-5-yl)vinyl)thiazole (160 mg, 0.245 mmol, 49.37%% yield) as a yellow oil. MS (ESI) m/z 588 [M+H]⁺.

### Step 4. 2-(1-(7-fluoro-2-(4-(4-(trifluoromethyl)pyridin-3-yl)-4H-1,2,4-triazol-3-yl)-1-((2-(trimethylsilyl)ethoxy)methyl)-1H-benzo[d]imidazol-5-yl)ethyl)thiazole

To a mixture of 2-(1-(7-fluoro-2-(4-(4-(trifluoromethyl)pyridin-3-yl)-4H-1,2,4-triazol-3-yl)-1-((2-(trimethylsilyl)ethoxy)methyl)-1H-benzo[d]imidazol-5-yl)vinyl)thiazole (1.00 eq, 160 mg, 0.272 mmol) in Methanol (15 mL) was added Pd/C (95 mg, 10%). The mixture was stirred for 1 hour at 25° C. under H₂ atmosphere. The reaction was filtered through a celite pad and the filtrate was concentrated to give 2-(1-(7-fluoro-2-(4-(4-(trifluoromethyl)pyridin-3-yl)-4H-1,2,4-triazol-3-yl)-1-((2-(trimethylsilyl)ethoxy)methyl)-1H-benzo[d]imidazol-5-yl)ethyl)thiazole (100 mg) as a yellow oil, which was used in the next step without further purification. MS (ESI) m/z 590 [M+H]⁺.

### Step 5. 2-(1-(7-fluoro-2-(4-(4-(trifluoromethyl)pyridin-3-yl)-4H-1,2,4-triazol-3-yl)-1H-benzo[d]imidazol-5-yl)ethyl)thiazole

To a mixture of 2-(1-(7-fluoro-2-(4-(4-(trifluoromethyl)pyridin-3-yl)-4H-1,2,4-triazol-3-yl)-1-((2-(trimethylsilyl)ethoxy)methyl)-1H-benzo[d]imidazol-5-yl)ethyl)thiazole (1.00 eq, 100 mg, 0.170 mmol) in DCM (10 mL) was added TFA (10 mL). The mixture was stirred for 1 hour at 25° C. The mixture was diluted with EtOAc, and washed with water. The organic layer was separated, dried over anhydrous sodium sulfate and concentrated under vacuum. The crude product was purified by prep-HPLC (H2O:ACN=30:70-100:0; collect products at 60% ACN) to give 2-(1-(7-fluoro-2-(4-(4-(trifluoromethyl)pyridin-3-yl)-4H-1,2,4-triazol-3-yl)-1H-benzo[d]imidazol-5-yl)ethyl)thiazole (33 mg, 0.0682 mmol, 40.24%% yield) as a white solid.

MS (ESI) m/z 460 [M+H]⁺.

¹H NMR (400 MHz, Chloroform-d) δ 12.96 (brs, 1H), 9.09 (d, J=5.2 Hz, 1H), 8.86 (s, 1H), 8.45 (s, 1H), 7.83 (d, J=5.2 Hz, 1H), 7.75 (d, J=3.2 Hz, 1H), 7.26-7.23 (m, 2H), 6.91 (d, J=11.2 Hz, 1H), 4.72-4.63 (m, 1H), 1.84 (d, J=6.8 Hz, 3H).

### Preparation of 6-(difluoro(pyridin-4-yl)methyl)-4-fluoro-2-(4-(4-(trifluoromethyl)pyridin-3-yl)-4H-1,2,4-triazol-3-yl)-1H-benzo[d]imidazole (464) Method 21

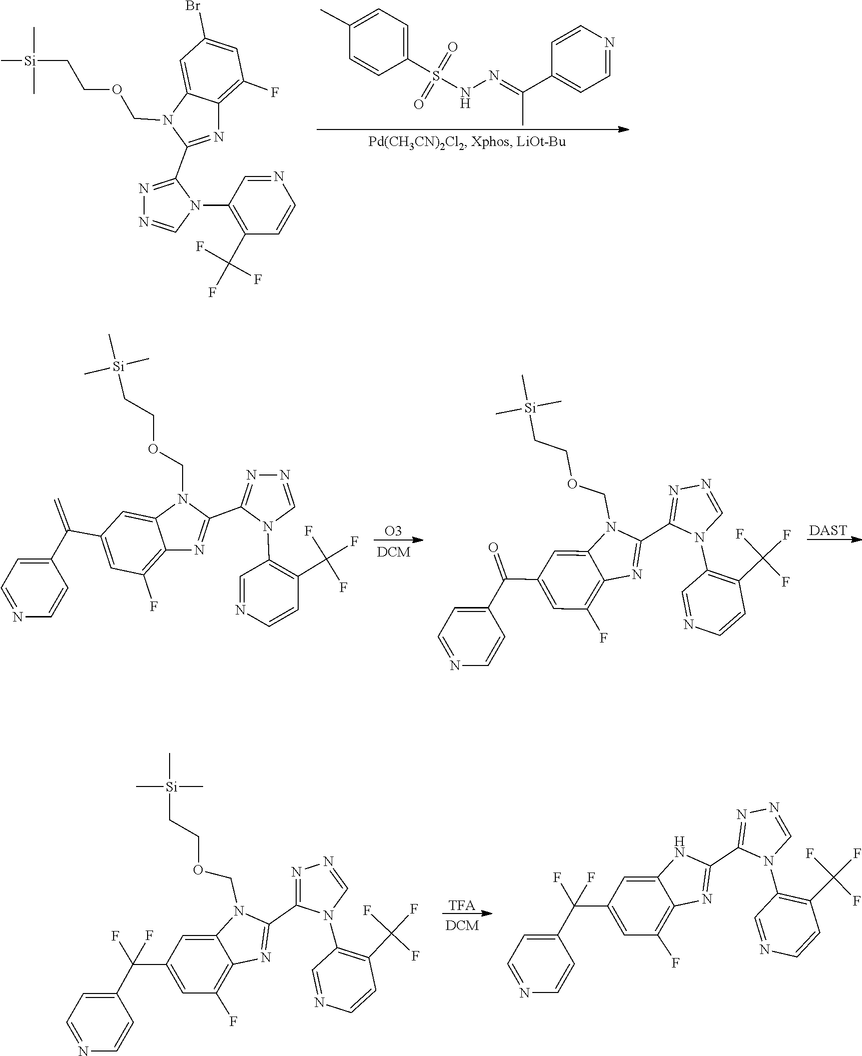

### Step 1. 2-[[4-fluoro-6-[1-(4-pyridyl)vinyl]-2-[4-[4-(trifluoromethyl)-3-pyridyl]-1,2,4-triazol-3-yl]benzimidazol-1-yl]methoxy]ethyl-trimethyl-silane

2-[[6-bromo-4-fluoro-2-[4-[4-(trifluoromethyl)-3-pyridyl]-1,2,4-triazol-3-yl]benzimidazol-1-yl]methoxy]ethyl-trimethyl-silane (1.00 eq, 2000 mg, 3.59 mmol), XPhos (0.100 eq, 171 mg, 0.359 mmol), Pd (CH3CN)2Cl2 (0.100 eq, 93 mg, 0.359 mmol) and t-BuOLi (3.00 eq, 862 mg, 10.8 mmol) were added to a solution of 4-methyl-N-[(E)-1-(4-pyridyl)ethylideneamino]benzenesulfonamide (2.00 eq, 2076 mg, 7.18 mmol) in 1,4-Dioxane (2 mL) under N₂ atmosphere. The mixture was stirred for 3 h at 90° C. and the precipitates were removed by filtration. The filtrates were concentrated under a vacuum to obtain a crude product. The crude product was purified by column chromatography with petroleum/ethyl acetate to give 2-[[4-fluoro-6-[1-(4-pyridyl)vinyl]-2-[4-[4-(trifluoromethyl)-3-pyridyl]-1,2,4-triazol-3-yl]benzimidazol-1-yl]methoxy]ethyl-trimethyl-silane (1780 mg, 3.06 mmol, 85.29% yield). MS (ESI) m/z 582 [M+H]⁺.

### Step 2. [7-fluoro-2-[4-[4-(trifluoromethyl)-3-pyridyl]-1,2,4-triazol-3-yl]-3-(2-trimethylsilylethoxymethyl)benzimidazol-5-yl]-(4-pyridyl)methanone

A solution of 2-[[4-fluoro-6-[1-(4-pyridyl)vinyl]-2-[4-[4-(trifluoromethyl)-3-pyridyl]-1,2,4-triazol-3-yl]benzimidazol-1-yl]methoxy]ethyl-trimethyl-silane (1.00 eq, 1780 mg, 3.06 mmol) in DCM (30 mL) was prepared in a three neck round bottomed flask provided with a drying tube and a gas dispersion tube. The solution was cooled to −78° C. The solution was saturated with O2. A stream of O3/O2 (approximately 1% of O3) was applied to the solution. After 15 minutes, the mixture became green-blue. The ozonizer was set to 0 V. The solution was purged with O2 for 15 minutes. After disappearance of starting material (judged by TLC), the reaction mixture was returned to room temperature. Me2S (3.00 eq, 570 mg, 9.18 mmol) was added to the reaction mixture. The resulting orange solution was stirred overnight. The resulting orange solution concentrated under reduced pressure. The residue was purified by chromatography on silica gel to give [7-fluoro-2-[4-[4-(trifluoromethyl)-3-pyridyl]-1,2,4-triazol-3-yl]-3-(2-trimethylsilylethoxymethyl)benzimidazol-5-yl]-(4-pyridyl)methanone (1210 mg, 2.07 mmol, 67.75% yield). MS (ESI) m/z 584 [M+H]⁺.

### Step 3. 2-[[6-[difluoro(4-pyridyl)methyl]-4-fluoro-2-[4-[4-(trifluoromethyl)-3-pyridyl]-1,2,4-triazol-3-yl]benzimidazol-1-yl]methoxy]ethyl-trimethyl-silane

A solution of [7-fluoro-2-[4-[4-(trifluoromethyl)-3-pyridyl]-1,2,4-triazol-3-yl]-3-(2-trimethylsilylethoxymethyl)benzimidazol-5-yl]-(4-pyridyl)methanone (1.00 eq, 130 mg, 0.223 mmol) in DAST (10 mL) was prepared. The mixture was stirred at room temperature overnight. After reaction completed, the residue was dissolved in DCM (50 mL) and washed with ice-cold saturated sodium bicarbonate solution (50 mL). The aqueous phase was washed with ethyl acetate (2×20 mL) and the combined organic extract was dried and concentrated. Purification by preparative HPLC afforded title compound 2-[[6-[difluoro(4-pyridyl)methyl]-4-fluoro-2-[4-[4-(trifluoromethyl)-3-pyridyl]-1,2,4-triazol-3-yl]benzimidazol-1-yl]methoxy]ethyl-trimethyl-silane (100 mg, 0.165 mmol, 74.13% yield) as off-white solid. MS (ESI) m/z 606 [M+H]⁺.

### Step 4. 6-(difluoro(pyridin-4-yl)methyl)-4-fluoro-2-(4-(4-(trifluoromethyl)pyridin-3-yl)-4H-1,2,4-triazol-3-yl)-1H-benzo[d]imidazole

To a stirred solution of 2-[[6-[difluoro(4-pyridyl)methyl]-4-fluoro-2-[4-[4-(trifluoromethyl)-3-pyridyl]-1,2,4-triazol-3-yl]benzimidazol-1-yl]methoxy]ethyl-trimethyl-silane (1.00 eq, 100 mg, 0.165 mmol) in DCM (5 mL) were added TFA dropwise at rt. The resulting mixture was stirred for 4 h. The reaction was monitored by LCMS. The resulting mixture was concentrated under vacuum. The residue was neutralized to pH 9 with K2CO3 (aq). The resulting mixture was extracted with DCM. The combined organic layers were washed with brine, dried over anhydrous Na₂SO₄. After filtration, the filtrate was concentrated under reduced pressure. The residue was purified by prep-TLC (MeOH/DCM=1/5) to afford 6-(difluoro(pyridin-4-yl)methyl)-4-fluoro-2-(4-(4-(trifluoromethyl)pyridin-3-yl)-4H-1,2,4-triazol-3-yl)-1H-benzo[d]imidazole (46 mg, 0.0968 mmol, 58.61% yield) as a white solid. MS (ESI) m/z 476 [M+H]⁺. ¹H NMR (400 MHz, Methanol-d4) δ 9.06 (d, J=5.2 Hz, 1H), 8.97 (d, J=4.4 Hz, 2H), 8.66 (s, 2H), 7.97 (d, J=5.2 Hz, 1H), 7.61-7.46 (m, 3H), 7.10 (s, 1H).

| | | | | |
|---|---|---|---|---|
| Example 401 Method 17 | 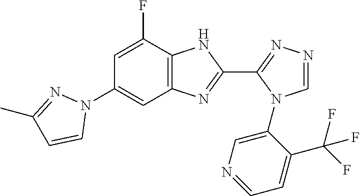 [SMILES: Cc1ccn(-c2cc(F)c3[nH]c(-c4nncn4-c4cnccc4C(F)(F)F)nc3c2)n1] | white solid, yield: 28% | ¹H NMR (400 MHz, Methanol-d₄) δ 9.08 (d, J = 5.2 Hz, 1H), 8.98 (d, J = 8.0 Hz, 2H), 8.09 (s, 1H), 8.00 (d, J = 5.2 Hz, 1H), 7.65 (s, 1H), 7.38 (s, 1H), 6.32 (d, J = 2.4 Hz, 1H), 2.34 (s, 3H). | 429 |
| | 7-fluoro-5-(3-methyl- | | | |
| | 1H-pyrazol-1-yl)-2-(4- | | | |
| | (4- | | | |
| | (trifluoromethyl)pyridin- | | | |
| | 3-yl)-4H-1,2,4-triazol- | | | |
| | 3-yl)-1H- | | | |
| | benzo[d]imidazole | | | |
| Example 402 Method 10 | 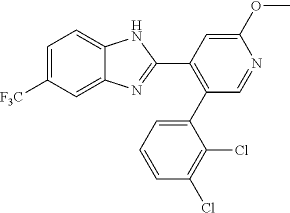 [SMILES: COc1cc(-c2nc3cc(C(F)(F)F)ccc3[nH]2)c(-c2cccc(Cl)c2Cl)cn1] | Off- white solid, yield: 5.8% | ¹¹H NMR (400 MHz, DMSO-d₆) δ 13.29 (s, 1H), 8.24 (s, 1H), 7.79 (s, 1H), 7.68 (s, 1H), 7.64 (dd, J = 7.6, 2.0 Hz, 2H), 7.52 (s, 1H), 7.43- 7.35 (m, 3H), 4.00 (s, 3H). | 438 |
| | 2-(5-(2,3- | | | |
| | dichlorophenyl)-2- | | | |
| | methoxypyridin-4-yl)- | | | |
| | 5-(trifluoromethyl)-1H- | | | |
| | benzo[d]imidazole | | | |
| Example 403 Method 17 | 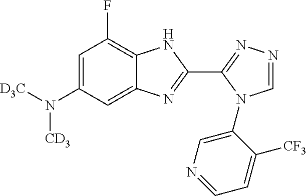 [SMILES: [2H]C([2H])([2H])N(c1cc(F)c2[nH]c(-c3nncn3-c3cnccc3C(F)(F)F)nc2c1)C([2H])([2H])[2H]] | yellow solid, yield: 9.5% | ¹H NMR (400 MHz, DMSO-d₆) δ 13.46- 13.19 (m, 1H), 9.08 (dd, J = 8.1, 5.4 Hz, 3H), 8.06 (d, J = 5.1 Hz, 1H), 6.53 (d, J = 14.4 Hz, 1H), 6.40 (d, J = 2.3 Hz, 1H). | 398 |
| | 7-fluoro-N,N- | | | |
| | bis(methyl-d₃)-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazol-5- | | | |
| | amine | | | |
| Example 404 Method 18 | 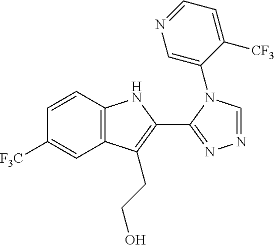 [SMILES: OCCc1c(-c2nncn2-c2cnccc2C(F)(F)F)[nH]c2ccc(C(F)(F)F)cc12] | white solid, yield: 8% | ¹H NMR (400 MHz, Methanol-d₄) δ 9.00- 8.89 (m, 3H), 7.99 (s, 1H), 7.91 (d, J = 5.2 Hz, 1H), 7.45-7.34 (m, 2H), 3.76 (t, J = 6.8 Hz, 2H), 3.04 (t, J = 6.8 Hz, 2H) | 442 |
| | 3-(2-tetrahydropyran-2- | | | |
| | yloxyethyl)-5- | | | |
| | (trifluoromethyl)-2-[4- | | | |
| | [4-(trifluoromethyl)-3- | | | |
| | pyridyl]-1,2,4-triazol-3- | | | |
| | yl]-1H-indole | | | |
| Example 405 Method 10 | 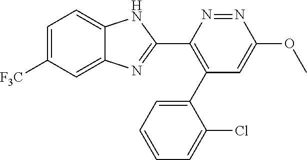 [SMILES: COc1cc(-c2ccccc2Cl)c(-c2nc3cc(C(F)(F)F)ccc3[nH]2)nn1] | white solid, yield: 27% | ¹H NMR (400 MHz, DMSO-d₆) δ 13.70 (s, 1H), 7.84-7.46 (m, 7H), 7.41(s, 1H), 3.34- 3.29 (m, 3H). | 405 |
| | 2-(4-(2-chlorophenyl)- | | | |
| | 6-methoxypyridazin-3- | | | |
| | yl)-5-(trifluoromethyl)- | | | |
| | 1H-benzo[d]imidazole | | | |
| Example 406 Method 10 | 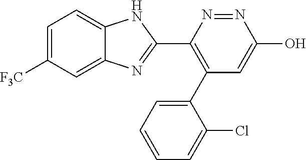 [SMILES: Oc1cc(-c2ccccc2Cl)c(-c2nc3cc(C(F)(F)F)ccc3[nH]2)nn1] | white solid, yield: 77% | ¹H NMR (400 MHz, DMSO-d₆) δ 13.88 (s, 1H), 13.28 (s, 1H), 7.75- 7.55 (m, 2H), 7.53- 7.37 (m, 5H), 6.98 (s, 1H). | 391 |
| | 5-(2-chlorophenyl)-6- | | | |
| | (5-(trifluoromethyl)- | | | |
| | 1H-benzo[d]imidazol-2- | | | |
| | yl)pyridazin-3-ol | | | |
| Example 407 Method 10 | 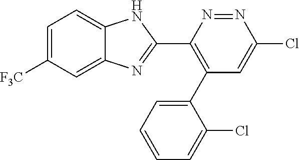 [SMILES: FC(F)(F)c1ccc2[nH]c(-c3nnc(Cl)cc3-c3ccccc3Cl)nc2c1] | white solid, yield: 47% | ¹H NMR (400 MHz, DMSO-d₆) δ 8.23 (s, 1H), 7.76 (s, 1H), 7.68 (d, J = 8.8 Hz, 1H), 7.60- 7.48 (m, 5H). | 409 |
| | 2-(6-chloro-4-(2- | | | |
| | chlorophenyl)pyridazin- | | | |
| | 3-yl)-5- | | | |
| | (trifluoromethyl)-1H- | | | |
| | benzo[d]imidazole | | | |
| Example 408 Method 10 | 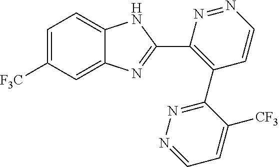 [SMILES: FC(F)(F)c1ccc2[nH]c(-c3nnccc3-c3nnccc3C(F)(F)F)nc2c1] | white solid, yield: 8% | ¹H NMR (400 MHz, DMSO-d₆) δ 14.17 (s, 1H), 9.76 (d, J = 5.6 Hz, 1H), 9.67 (d, J = 5.2 Hz, 1H), 8.38 (d, J = 5.6 Hz, 1H), 8.20 (d, J = 5.2 Hz, 1H), 7.90-7.35 (m, 3H). | 411 |
| | 5-(trifluoromethyl)-2- | | | |
| | (4-(trifluoromethyl)- | | | |
| | [3,4′-bipyridazin]-3′-yl)- | | | |
| | 1H-benzo[d]imidazole | | | |
| Example 409 Method 9 | 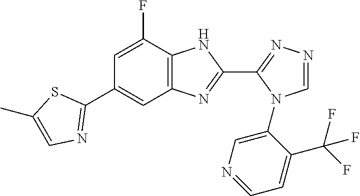 [SMILES: Cc1cnc(-c2cc(F)c3[nH]c(-c4nncn4-c4cnccc4C(F)(F)F)nc3c2)s1] | white solid, yield: 4% | [M + H]⁺. ¹H NMR (400 MHz, MeOD) δ 9.09 (d, J = 5.2 Hz, 1H), 8.99 (s, 1H), 8.98 (s, 1H), 8.00 (d, J = 5.2 Hz, 1H), 7.87 (s, 1H), 7.52 (s, 1H), 7.49-7.39 (m, 1H), 2.52 (s, 3H). | 446 |
| | 2-[7-fluoro-2-[4-[4- | | | |
| | (trifluoromethyl)-3- | | | |
| | pyridyl]-1,2,4-triazol-3- | | | |
| | yl]-1H-benzimidazol-5- | | | |
| | yl]-5-methyl-thiazole. | | | |
| Example 410 Method 13 | 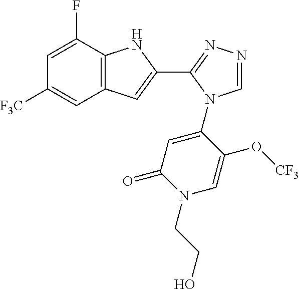 [SMILES: O=c1cc(-n2cnnc2-c2cc3cc(C(F)(F)F)cc(F)c3[nH]2)c(OC(F)(F)F)cn1CCO] | white solid, | ¹H NMR (500 MHz, METHANOL-d₄) δ 8.87 (s, 1H), 8.16 (s, 1H), 7.77 (s, 1H), 7.25 (dd, J = 0.9, 11.1 Hz, 1H), 6.98 (s, 1H), 6.75 (d, J = 2.9 Hz, 1H), 4.23-4.17 (m, 2H), 3.92-3.87 (m, 2H) | 492 |
| | 4-(3-(7-fluoro-5- | | | |
| | (trifluoromethyl)-1H- | | | |
| | indol-2-yl)-4H-1,2,4- | | | |
| | triazol-4-yl)-1-(2- | | | |
| | hydroxyethyl)-5- | | | |
| | (trifluoromethoxy) | | | |
| | pyridin-2(1H)-one | | | |
| Example 411 Method 13 | 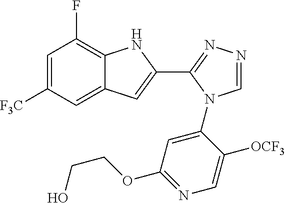 [SMILES: OCCOc1cc(-n2cnnc2-c2cc3cc(C(F)(F)F)cc(F)c3[nH]2)c(OC(F)(F)F)cn1] | white solid, | ¹H NMR (500 MHz, METHANOL-d₄) δ 8.89 (s, 1H), 8.43 (d, J = 0.9 Hz, 1H), 7.71 (s, 1H), 7.31 (s, 1H), 7.24 (dd, J = 0.8, 11.1 Hz, 1H), 6.44 (d, J = 2.9 Hz, 1H), 4.52- 4.50 (m, 2H), 3.92- 3.90 (m, 2H) | 492 |
| | 2-((4-(3-(7-fluoro-5- | | | |
| | (trifluoromethyl)-1H- | | | |
| | indol-2-yl)-4H-1,2,4- | | | |
| | triazol-4-yl)-5- | | | |
| | (trifluoromethoxy) | | | |
| | pyridin-2-yl)oxy) | | | |
| | ethanol | | | |
| Example 412 Method 20 mixture of stereoisomers | 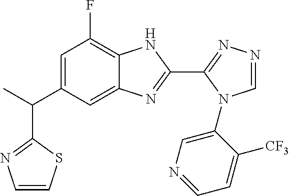 [SMILES: CC(c1cc(F)c2[nH]c(-c3nncn3-c3cnccc3C(F)(F)F)nc2c1)c1nccs1] | white solid, yield: 40% | ¹H NMR (400 MHz, Chloroform-d) δ 12.96 (brs, 1H), 9.09 (d, J = 5.2 Hz, 1H), 8.86 (s, 1H), 8.45 (s, 1H), 7.83 (d, J = 5.2 Hz, 1H), 7.75 (d, J = 3.2 Hz, 1H), 7.26- 7.23 (m, 2H), 6.91 (d, J = 11.2 Hz, 1H), 4.72- 4.63 (m, 1H), 1.84 (d, J = 6.8 Hz, 3H). | 460 |
| | 2-(1-(7-fluoro-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazol-5- | | | |
| | yl)ethyl)thiazole | | | |
| Example 413 Method 19 | 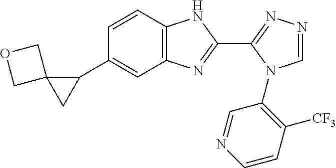 [SMILES: FC(F)(F)c1ccncc1-n1cnnc1-c1nc2cc(C3CC34COC4)ccc2[nH]1] | white solid, yield: 5% | ¹H NMR (400 MHz, Chloroform-d) δ 13.02- 12.51 (m, 1H), 9.19- 9.08 (m, 1H), 8.88 (s, 1H), 8.47 (s, 1H), 7.86 (br d, J = 4.9 Hz, 1H), 7.25-7.14 (m, 1H), 6.65- 6.49 (m, 1H), 4.91- 4.71 (m, 3H), 4.53 (br s, 1H), 2.27-2.15 (m, 1H), 1.37-1.28 (m, 1H), 1.15 | 431 |
| | 7-fluoro-5-(5- | | (br t, J = 5.7 Hz, 1H). | |
| | oxaspiro[2.3]hexan-2- | | | |
| | yl)-2-[4-[4- | | | |
| | (trifluoromethyl)-3- | | | |
| | pyridyl]-1,2,4-triazol-3- | | | |
| | yl]-1H-benzimidazole | | | |
| Example 414 Method 19 | 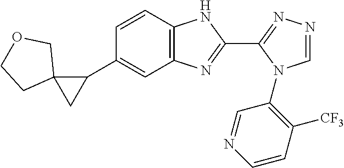 [SMILES: FC(F)(F)c1ccncc1-n1cnnc1-c1nc2cc(C3CC34CCOC4)ccc2[nH]1] | white solid, yield: 7% | ¹H NMR (400 MHz, Chloroform-d) δ 9.12 (d, J = 4.9 Hz, 1H), 8.89 (s, 1H), 8.48 (s, 1H), 7.88 (d, J = 5.0 Hz, 1H), 7.25 (br s, 1H), 6.74 (br d, J = 10.6 Hz, 1H), 4.09- 3.90 (m, 2H), 3.56 (d, J = 8.6 Hz, 1H), 3.35 (br d, J = 8.3 Hz, 1H), 2.33 (dd, J = 6.5, 8.0 Hz, | 445 |
| | 7-fluoro-5-(5- | | 1H), 2.14-1.95 (m, 2H), | |
| | oxaspiro[2.4]heptan-2- | | 1.37-1.27 (m, 1H), 1.20 | |
| | yl)-2-[4-[4- | | (t, J = 5.7 Hz, 1H). | |
| | (trifluoromethyl)-3- | | | |
| | pyridyl]-1,2,4-triazol-3- | | | |
| | yl]-1H-benzimidazole | | | |
| Example 415 Method 13 | 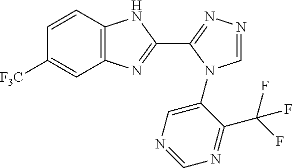 [SMILES: FC(F)(F)c1ccc2[nH]c(-c3nncn3-c3cncnc3C(F)(F)F)nc2c1] | white solid, yield: 4.2% | ¹H NMR (400 MHz, DMSO-d₆) δ 14.22 (d, J = 13.9 Hz, 1H), 9.72 (s, 1H), 9.51 (d, J = 2.4 Hz, 1H), 9.15 (d, J = 1.8 Hz, 1H), 7.79 (d, J = 19.9 Hz, 1H), 7.73-7.45 (m, 2H). | 400 |
| | 5-(trifluoromethyl)-2- | | | |
| | (4-(4-(trifluoro- | | | |
| | methyl)pyrimidin- | | | |
| | 5-yl)-4H-1,2,4- | | | |
| | triazol-3-yl)-1H- | | | |
| | benzo[d]imidazole | | | |
| Example 416 Method 13 | 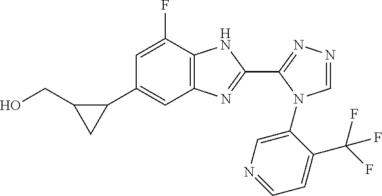 [SMILES: OCC1CC1c1cc(F)c2[nH]c(-c3nncn3-c3cnccc3C(F)(F)F)nc2c1] | white solid, yield: 14% | ¹H NMR (400 MHz, DMSO-d₆) δ 13.78 (s, 1H), 9.12 (d, J = 5.2 Hz, 1H), 9.12-9.07 (m, 2H), 8.07 (d, J = 4.8 Hz, 1H), 7.13 (s, 1H), 6.87 (d, J = 10.1 Hz, 1H), 4.61 (s, 1H), 3.18-3.06 (m, 1H), 3.07-2.96 (m, 1H), 2.33-2.23 (m, 1H), 1.40-1.28 (m, 1H), 0.97 (td, J = 8.4, | 419 |
| | (2-(7-fluoro-2-(4-(4- | | 5.0 Hz, 1H), 0.89-0.77 | |
| | (trifluoromethyl)pyridin- | | (m, 1H). | |
| | 3-yl)-4H-1,2,4-triazol- | | | |
| | 3-yl)-1H- | | | |
| | benzo[d]imidazol-5- | | | |
| | yl)cyclopropyl)methanol | | | |
| Example 417 Method 17 | 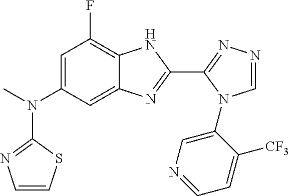 [SMILES: CN(c1cc(F)c2[nH]c(-c3nncn3-c3cnccc3C(F)(F)F)nc2c1)c1nccs1] | white solid, yield: 7.5% | ¹H NMR (400 MHz, Chloroform-d) δ 9.13 (d, J = 5.2 Hz, 1H), 8.94- 8.79 (m, 1H), 8.52-8.47 (m, 1H), 8.00-7.80 (m, 2H), 7.34 (d, J = 11.6 Hz, 1H), 6.95 (d, J = 10.0 Hz, 1H), 6.54 (s, 1H), 3.92-3.78 (m, 3H). | 461 |
| | N-(7-fluoro-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazol-5-yl)- | | | |
| | N-methylthiazol-2- | | | |
| | amine | | | |
| Example 418 Method 10 | 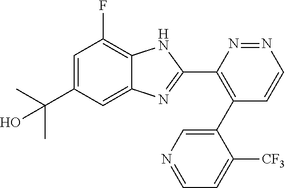 [SMILES: CC(C)(O)c1cc(F)c2[nH]c(-c3nnccc3-c3cnccc3C(F)(F)F)nc2c1] | white solid, | ¹H NMR (400 MHz, Methanol-d₄) δ 9.37 (d, J = 5.2 Hz, 1H), 8.92 (d, J = 5.3 Hz, 1H), 8.68 (s, 1H), 7.83 (t, J = 5.1 Hz, 2H), 7.49 (d, J = 1.4 Hz, 1H), 7.00 (dd, J = 12.6, 1.4 Hz, 1H), 1.55 (s, 6H). | 418 |
| | 2-(4-fluoro-2-(4-(4- | | | |
| | (trifluoromethyl)pyridin- | | | |
| | 3-yl)pyridazin-3-yl)- | | | |
| | 1H-benzo[d]imidazol-6- | | | |
| | yl)propan-2-ol | | | |
| Example 419 Method 14 | 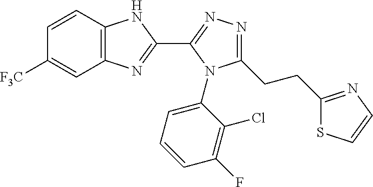 [SMILES: Fc1cccc(-n2c(CCc3nccs3)nnc2-c2nc3cc(C(F)(F)F)ccc3[nH]2)c1Cl] | white solid, yield: 10% | ¹H NMR (400 MHz, DMSO-d₆) δ 7.81-7.72 (m, 2H), 7.70-7.62 (m, 4H), 7.56 (d, J = 3.2 Hz, 1H), 7.54-7.48 (m, 1H), 3.51 (t, J = 7.6 Hz, 2H), 3.10-2.98 (m, 2H). | 493 |
| | 2-(2-(4-(2-chloro-3- | | | |
| | fluorophenyl)-5-(5- | | | |
| | (trifluoromethyl)-1H- | | | |
| | benzo[d]imidazol-2-yl)- | | | |
| | 4H-1,2,4-triazol-3- | | | |
| | yl)ethyl)thiazole | | | |
| Example 420 Method 10 | 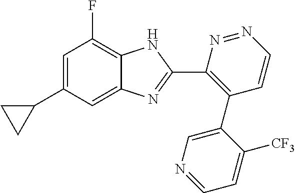 [SMILES: Fc1cc(C2CC2)cc2nc(-c3nnccc3-c3cnccc3C(F)(F)F)[nH]c12] | white solid, | ¹H NMR (400 MHz, DMSO-d₆) δ 13.66 (s, 1H), 9.49 (d, J = 5.2 Hz, 1H), 8.96 (d, J = 5.2 Hz, 1H), 8.75 (s, 1H), 7.92 (dd, J = 20.9, 5.3 Hz, 2H), 7.06 (s, 1H), 6.62 (d, J = 12.3 Hz, 1H), 2.08-1.97 (m, 1H), 1.01- 0.92 (m, 2H), 0.72- 0.63 (m, 2H). | 400 |
| | 6-cyclopropyl-4-fluoro- | | | |
| | 2-(4-(4- | | | |
| | (trifluoromethyl)pyridin- | | | |
| | 3-yl)pyridazin-3-yl)- | | | |
| | 1H-benzo[d]imidazole | | | |
| Example 421 Method 14 | 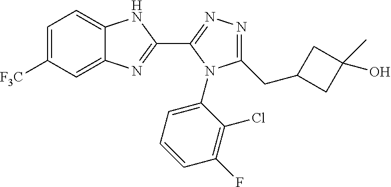 [SMILES: CC1(O)CC(Cc2nnc(-c3nc4cc(C(F)(F)F)ccc4[nH]3)n2-c2cccc(F)c2Cl)C1] | yellow solid, yield: 39.6% | ¹H NMR (400 MHz, DMSO-d₆) δ 13.92 (br s, 1H), 7.80- 7.71 (m, 2H), 7.70- 7.61 (m, 3H), 7.51 (m, 1H), 4.81 (br s, 1H), 2.75-2.58 (m, 2H), 2.22-1.95 (m, 3H), 1.76-1.59 (m, 2H), 1.17 (s, 3H). | 480.2 |
| | 3-((4-(2-chloro-3- | | | |
| | fluorophenyl)-5-(5- | | | |
| | (trifluoromethyl)-1H- | | | |
| | benzo[d]imidazol-2-yl)- | | | |
| | 4H-1,2,4-triazol-3- | | | |
| | yl)methyl)-1- | | | |
| | methylcyclobutan-1-ol | | | |
| Example 422 Method 8 | 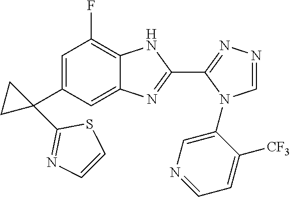 [SMILES: Fc1cc(C2(c3nccs3)CC2)cc2nc(-c3nncn3-c3cnccc3C(F)(F)F)[nH]c12] | white solid, yield: 21% | ¹H NMR (400 MHz, MeOD) δ 9.07 (d, J = 5.1 Hz, 1H), 8.97 (s, 1H), 8.95 (s, 1H), 7.99 (d, J = 5.1 Hz, 1H), 7.59 (d, J = 3.4 Hz, 1H), 7.50- 7.33 (m, 1H), 7.29 (d, J = 3.4 Hz, 1H), 7.02 (d, J = 11.4 Hz, 1H), 1.79- 1.64 (m, 2H), 1.55-1.45 (m, 2H). | 472 |
| | 2-[1-[7-fluoro-2-[4-[4- | | | |
| | (trifluoromethyl)-3- | | | |
| | pyridyl]-1,2,4-triazol-3- | | | |
| | yl]-1H-benzimidazol-5- | | | |
| | yl]cyclopropyl]thiazole | | | |
| Example 423 Method 8 | 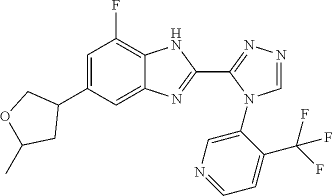 [SMILES: CC1CC(c2cc(F)c3[nH]c(-c4nncn4-c4cnccc4C(F)(F)F)nc3c2)CO1] | white solid, yield: 51% | ¹H NMR (400 MHz, Chloroform-d) δ 13.12- 12.98 (m, 1H), 9.11 (d, J = 4.9 Hz, 1H), 8.91- 8.85 (m, 1H), 8.51- 8.41 (m, 1H), 7.88-7.82 (m, 1H), 7.73-7.68 (m, 1H), 7.00-6.81 (m, 1H), 4.18-4.13 (m, 1H), 3.94- 3.76 (m, 1H), 3.67- 3.46 (m, 1H), 2.60-2.41 (m, 1H), 1.75-1.54 (m, | 433 |
| | 7-fluoro-5-(5- | | 2H), 1.45-1.27 (m, 3H). | |
| | methyltetrahydrofuran- | | | |
| | 3-yl)-2-[4-[4- | | | |
| | (trifluoromethyl)-3- | | | |
| | pyridyl]-1,2,4-triazol-3- | | | |
| | yl]-1H-benzimidazole | | | |
| Example 424 Method 8 | 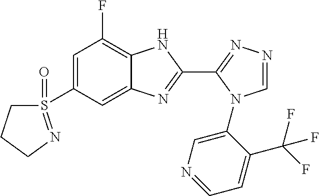 [SMILES: O=S1(c2cc(F)c3[nH]c(-c4nncn4-c4cnccc4C(F)(F)F)nc3c2)=NCCC1] | white solid, yield: 67% | ¹H NMR (500 MHz, Methanol-d₄) δ 9.09 (d, J = 5.0 Hz, 1H), 8.99 (d, J = 9.5 Hz, 2H), 8.00 (d, J = 5.5 Hz, 1H), 7.96 (br s, 1H), 7.50 (br s, 1H), 4.00- 3.93 (m, 1H), 3.82-3.76 (m, 1H), 3.53-3.46 (m, 2H), 2.47-2.32 (m, 2H) | 452 |
| | 1-[7-fluoro-2-[4-[4- | | | |
| | (trifluoromethyl)-3- | | | |
| | pyridyl]-1,2,4-triazol-3- | | | |
| | yl]-1H-benzimidazol-5- | | | |
| | yl]-4,5-dihydro-3H- | | | |
| | isothiazole 1-oxide | | | |
| Example 425 Method 18 | 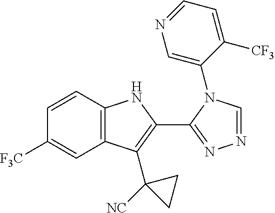 [SMILES: N#CC1(c2c(-c3nncn3-c3cnccc3C(F)(F)F)[nH]c3ccc(C(F)(F)F)cc23)CC1] | white solid, yield: 3% | ¹H NMR (500 MHz, Methanol-d₄) δ 9.06- 9.02 (m, 1H), 9.00-8.97 (m, 2H), 8.02 (s, 1H), 7.91 (d, J = 5.2 Hz, 1H), 7.57 (q, J = 8.6 Hz, 2H), 1.65 (br d, J = 2.4 Hz, 2H), 1.29 (br s, 2H) | 463 |
| | 1-[5-(trifluoromethyl)- | | | |
| | 2-[4-[4- | | | |
| | (trifluoromethyl)-3- | | | |
| | pyridyl]-1,2,4-triazol-3- | | | |
| | yl]-1H-indol-3- | | | |
| | yl]cyclopropane- | | | |
| | carbonitrile | | | |
| Example 426 Method 18 | 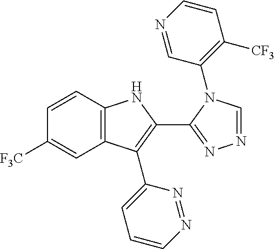 [SMILES: FC(F)(F)c1ccc2[nH]c(-c3nncn3-c3cnccc3C(F)(F)F)c(-c3cccnn3)c2c1] | white solid, yield: 31% | ¹H NMR (500 MHz, Methanol-d₄) δ 9.19 (br d, J = 4.9 Hz, 1H), 9.09 (br s, 1H), 8.95 (s, 1H), 8.83 (d, J = 5.2 Hz, 1H), 8.38 (s, 1H), 7.95 (s, 1H), 7.76-7.70 (m, 3H), 7.62 (dd, J = 1.3, 8.8 Hz, 1H) | 476 |
| | 3-pyridazin-3-yl-5- | | | |
| | (trifluoromethyl)-2-[4- | | | |
| | [4-(trifluoromethyl)-3- | | | |
| | pyridyl]-1,2,4-triazol-3- | | | |
| | yl]-1H-indole | | | |
| Example 427 Method 18 | 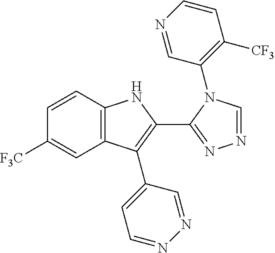 [SMILES: FC(F)(F)c1ccc2[nH]c(-c3nncn3-c3cnccc3C(F)(F)F)c(-c3ccnnc3)c2c1] | white solid, yield: 46% | ¹H NMR (400 MHz, Methanol-d₄) δ 9.13 (d, J = 5.5 Hz, 1H), 9.06 (s, 1H), 8.86 (s, 1H), 8.74 (d, J = 5.1 Hz, 1H), 8.33 (s, 1H), 7.87 (s, 1H), 7.74 (dd, J = 2.3, 5.5 Hz, 1H), 7.64 (d, J = 5.0 Hz, 1H), 7.61-7.56 (m, 1H), 7.54-7.49 (m, 1H) | 476 |
| | 3-pyridazin-4-yl-5- | | | |
| | (trifluoromethyl)-2-[4- | | | |
| | [4-(trifluoromethyl)-3- | | | |
| | pyridyl]-1,2,4-triazol-3- | | | |
| | yl]-1H-indole | | | |
| Example 428 Method 18 | 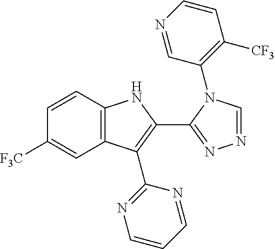 [SMILES: FC(F)(F)c1ccc2[nH]c(-c3nncn3-c3cnccc3C(F)(F)F)c(-c3ncccn3)c2c1] | white solid, yield: 43% | ¹H NMR (400 MHz, Methanol-d₄) δ 9.00 (s, 1H), 8.81 (s, 1H), 8.74- 8.68 (m, 3H), 8.65 (s, 1H), 7.69 (d, J = 5.1 Hz, 1H), 7.67-7.62 (m, 1H), 7.57-7.51 (m, 1H), 7.22 (t, J = 4.9 Hz, 1H) | 476 |
| | 3-pyrimidin-2-yl-5- | | | |
| | (trifluoromethyl)-2-[4- | | | |
| | [4-(trifluoromethyl)-3- | | | |
| | pyridyl]-1,2,4-triazol-3- | | | |
| | yl]-1H-indole | | | |
| Example 429 Method 13 | 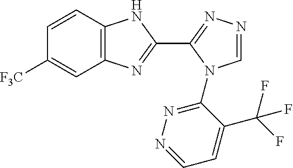 [SMILES: FC(F)(F)c1ccc2[nH]c(-c3nncn3-c3nnccc3C(F)(F)F)nc2c1] | white solid, yield: 28% | ¹H NMR (400 MHz, DMSO-d₆) δ 14.21 (s, 1H), 9.92 (d, J = 5.2 Hz, 1H), 9.34 (s, 1H), 8.61 (d, J = 5.2 Hz, 1H), 7.74 (s, 1H), 7.65 (d, J = 8.5 Hz, 1H), 7.52 (dd, J = 8.6, 1.8 Hz, 1H). | 400 |
| | 5-(trifluoromethyl)-2- | | | |
| | (4-(4-(trifluoromethyl) | | | |
| | pyridazin- | | | |
| | 3-yl)-4H-1,2,4- | | | |
| | triazol-3-yl)-1H- | | | |
| | benzo[d]imidazole | | | |
| Example 430 Method 20 | 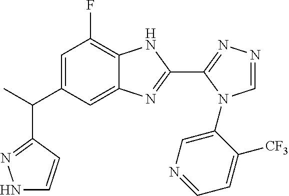 [SMILES: CC(c1cc(F)c2[nH]c(-c3nncn3-c3cnccc3C(F)(F)F)nc2c1)c1cc[nH]n1] | white solid, yield: 28.7% | ¹H NMR (400 MHz, Chloroform-d) δ 13.54 (brs, 1H), 9.05 (d, J = 5.2 Hz, 1H), 8.78 (d, J = 9.2 Hz, 1H), 8.38 (s, 1H), 7.77 (d, J = 5.2 Hz, 1H), 7.70-7.55 (m, 2H), 6.85-6.68 (m, 1H), 6.44-6.25 (m, 1H), 4.59-4.37 (m, 1H), 1.73 (d, J = 7.2 Hz, 3H). | 443 |
| | 5-(1-(1H-pyrazol-5- | | | |
| | yl)ethyl)-7-fluoro-2-(4- | | | |
| | (4-(trifluoromethyl) | | | |
| | pyridin- | | | |
| | 3-yl)-4H-1,2,4-triazol- | | | |
| | 3-yl)-1H- | | | |
| | benzo[d]imidazole | | | |
| Example 431 Method 20 | 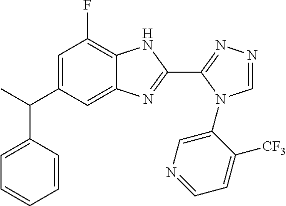 [SMILES: CC(c1ccccc1)c1cc(F)c2[nH]c(-c3nncn3-c3cnccc3C(F)(F)F)nc2c1] | white solid, yield: 23.9% | ¹H NMR (400 MHz, Chloroform-d) δ 11.56 (brs, 1H), 9.08 (d, J = 5.2 Hz, 1H), 8.83 (s, 1H), 8.38 (s, 1H), 7.81 (d, J = 5.2 Hz, 1H), 7.38- 7.31 (m, 1H), 7.30- 7.27 (m, 2H), 7.23-7.15 (m, 3H), 6.79 (s, 1H), 4.29-4.17 (m, 1H), 1.66 (d, J = 7.2 Hz, 3H). | 453 |
| | 7-fluoro-5-(1- | | | |
| | phenylethyl)-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazole | | | |
| Example 432 Method 20 | 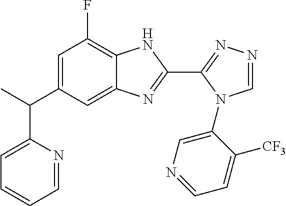 [SMILES: CC(c1cc(F)c2[nH]c(-c3nncn3-c3cnccc3C(F)(F)F)nc2c1)c1ccccn1] | white solid, yield: 35.2% | ¹H NMR (400 MHz, Chloroform-d) δ 9.08 (d, J = 5.2 Hz, 1H), 8.86- 8.81 (m, 2H), 8.50 (s, 1H), 8.10-8.02 (m, 1H), 7.82 (d, J = 5.2 Hz, 1H), 7.78-7.73 (m, 1H), 7.56 (t, J = 6.4 Hz, 1H), 7.50 (t, J = 6.8 Hz, 1H), 6.91-6.84 (m, 1H), 4.98-4.86 (m, 1H), 1.84 (d, J = 7.2 Hz, 3H). | 454 |
| | 7-fluoro-5-(1-(pyridin- | | | |
| | 2-yl)ethyl)-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazole | | | |
| Example 433 Method 20 single unknown enantiomer | 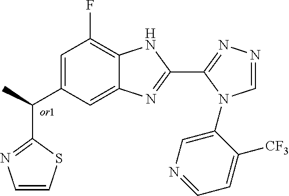 [SMILES: C[C@@H](c1cc(F)c2[nH]c(-c3nncn3-c3cnccc3C(F)(F)F)nc2c1)c1nccs1] | white solid, | ¹H NMR (400 MHz, Chloroform-d) δ 11.41 (brs, 1H), 9.08 (d, J = 5.2 Hz, 1H), 8.83 (s, 1H), 8.43-8.38 (m, 1H), 7.81 (d, J = 5.2 Hz, 1H), 7.75-7.71 (m, 1H), 7.58-7.47 (m, 1H), 7.25-7.22 (m, 1H), 6.95-6.83 (m, 1H), 4.71-4.57 (m, 1H), 1.92-1.76 (m, 3H). | 460 CHIRAL PAK AD-H, 2 cm * 25 cm, 5 um, 40% IPA (NH4OH 0.2%): 60% CO2 Rt = 6.191 min |
| | 2-(1-(7-fluoro-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazol-5- | | | |
| | yl)ethyl)thiazole | | | |
| Example 434 Method 20 single unknown enantiomer | 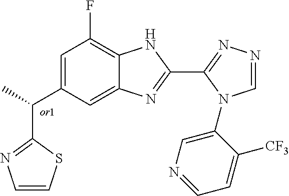 [SMILES: C[C@H](c1cc(F)c2[nH]c(-c3nncn3-c3cnccc3C(F)(F)F)nc2c1)c1nccs1] | white solid, | ¹H NMR (400 MHz, Chloroform-d) δ 11.41 (brs, 1H), 9.08 (d, J = 5.2 Hz, 1H), 8.84 (s, 1H), 8.43 (s, 1H), 7.82 (d, J = 5.2 Hz, 1H), 7.76 (d, J = 3.4 Hz, 1H), 7.71- 7.57 (m, 1H), 7.25- 7.22 (m, 1H), 6.98-6.86 (m, 1H), 4.81-4.66 (m, 1H), 1.86 (d, J = 7.2 Hz, 3H). | 460 CHIRAL PAK AD-H, 2 cm * 25 cm, 5 um, 40% IPA (NH4OH 0.2%): 60% CO2 Rt = 7.419 min |
| | 2-(1-(7-fluoro-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazol-5- | | | |
| | yl)ethyl)thiazole | | | |
| Example 435 Method 20 | 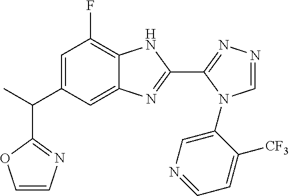 [SMILES: CC(c1cc(F)c2[nH]c(-c3nncn3-c3cnccc3C(F)(F)F)nc2c1)c1ncco1] | white solid, yield: 15.9% | ¹H NMR (400 MHz, DMSO-d₆) δ 9.15-9.14 (m, 1H), 9.11-9.09 (m, 2H), 8.07 (d, J = 5.2 Hz, 1H), 8.01-7.95 (m, 1H), 7.21 (s, 1H), 7.15 (s, 1H), 6.89 (d, J = 11.8 Hz, 1H), 4.49 (d, J = 7.2 Hz, 1H), 1.61 (d, J = 7.2 Hz, 3H). | 444 |
| | 2-(1-(7-fluoro-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazol-5- | | | |
| | yl)ethyl)oxazole | | | |
| Example 436 Method 17 | 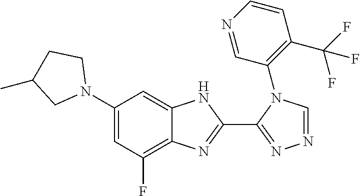 [SMILES: CC1CCN(c2cc(F)c3nc(-c4nncn4-c4cnccc4C(F)(F)F)[nH]c3c2)C1] | yellow solid, yield: 55.2% | ¹H NMR (400 MHz, Methanol-d₄) δ 9.04 (d, J = 5.2 Hz, 1H), 8.93 (s, 1H), 8.85 (s, 1H), 7.96 (d, J = 5.2 Hz, 1H), 6.34-6.23 (m, 2H), 3.51-3.37 (m, 2H), 3.35-3.28 (m, 2H), 2.41 (m, 1H), 2.21-2.13 (m, 1H), 1.64 (m, 1H), 1.14 (d, J = 6.4 Hz, 3H). | 432.1 |
| | 4-fluoro-6-(3- | | | |
| | methylpyrrolidin-1-yl)- | | | |
| | 2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazole | | | |
| Example 437 Method 13 | 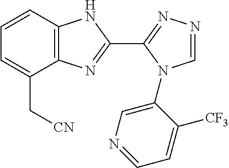 [SMILES: N#CCc1cccc2[nH]c(-c3nncn3-c3cnccc3C(F)(F)F)nc12] | white solid, yield: 13% | ¹H NMR (400 MHz, DMSO-d₆) δ 9.14-9.00 (m, 3H), 8.05 (d, J = 5.0 Hz, 1H), 7.48 (s, 1H), 7.27 (d, J = 77.8 Hz, 2H), 3.73 (s, 2H). | 370 |
| | 2-(2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazol-4- | | | |
| | yl)acetonitrile | | | |
| Example 438 Method 10 | 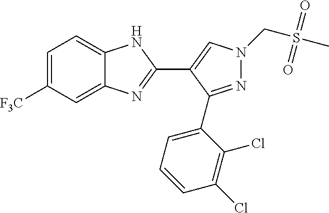 [SMILES: CS(=O)(=O)Cn1cc(-c2nc3cc(C(F)(F)F)ccc3[nH]2)c(-c2cccc(Cl)c2Cl)n1] | white solid, yield: 3% | ¹H NMR (400 MHz, DMSO-d₆) δ 12.96 (d, J = 23.5 Hz, 1H), 8.64 (s, 1H), 7.76 (dd, J = 7.9, 1.8 Hz, 2H), 7.64 (d, J = 8.4 Hz, 1H), 7.55-7.42 (m, 3H), 5.99 (s, 2H), 3.11 (s, 3H). | 489 |
| | 2-(3-(2,3- | | | |
| | dichlorophenyl)-1- | | | |
| | ((methylsulfonyl)methyl)- | | | |
| | 1H-pyrazol-4-yl)-5- | | | |
| | (trifluoromethyl)-1H- | | | |
| | benzo[d]imidazole | | | |
| Example 439 Method 10 | 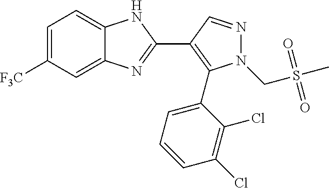 [SMILES: CS(=O)(=O)Cn1ncc(-c2nc3cc(C(F)(F)F)ccc3[nH]2)c1-c1cccc(Cl)c1Cl] | white solid, yield: 1% | ¹H NMR (400 MHz, DMSO-d₆) δ 8.45 (s, 1H), 7.86 (dd, J = 5.5, 4.1 Hz, 1H), 7.75 (d, J = 1.7 Hz, 1H), 7.63 (d, J = 8.4 Hz, 1H), 7.59-7.53 (m, 2H), 7.44 (dd, J = 8.5, 1.8 Hz, 1H), 5.81 (d, J = 14.9 Hz, 1H), 5.23 (d, J = 14.8 Hz, 1H), 3.15 (s, 3H). | 489 |
| | 2-(5-(2,3- | | | |
| | dichlorophenyl)-1- | | | |
| | ((methylsulfonyl)methyl)- | | | |
| | 1H-pyrazol-4-yl)-5- | | | |
| | (trifluoromethyl)-1H- | | | |
| | benzo[d]imidazole | | | |
| Example 440 Method 8 | 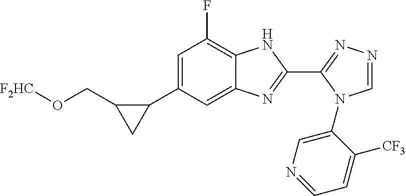 [SMILES: Fc1cc(C2CC2COC(F)F)cc2nc(-c3nncn3-c3cnccc3C(F)(F)F)[nH]c12] | white solid, yield: 44% | ¹H NMR (400 MHz, DMSO-d₆) δ 13.79 (d, J = 19.6 Hz, 1H), 9.14- 9.07 (m, 3H), 8.07 (dd, J = 4.8, 3.3 Hz, 1H), 7.17 (s, 1H), 6.87 (d, J = 11.9 Hz, 1H), 6.29-5.77 (m, 1H), 3.69 (td, J = 15.2, 3.8 Hz, 1H), 3.16-3.01 (m, 1H), 2.41-2.26 (m, 1H), 1.49-1.33 (m, 1H), 1.11-1.00 (m, | 469 |
| | 5-(2- | | 1H), 0.98-0.84 (m, | |
| | ((difluoromethoxy) | | 1H). | |
| | methyl)cyclopropyl)-7- | | | |
| | fluoro-2-(4-(4- | | | |
| | (trifluoromethyl)- | | | |
| | pyridin-3-yl)-4H-1,2,4- | | | |
| | triazol-3-yl)-1H- | | | |
| | benzo[d]imidazole | | | |
| Example 441 Method 8 | 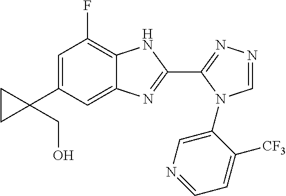 [SMILES: OCC1(c2cc(F)c3[nH]c(-c4nncn4-c4cnccc4C(F)(F)F)nc3c2)CC1] | white solid, yield: 10% | ¹H NMR (400 MHz, DMSO-d₆) δ 13.78 (s, 1H), 9.13-9.07 (m, 3H), 8.06 (d, J = 5.1 Hz, 1H), 7.25 (s, 1H), 6.89 (d, J = 11.0 Hz, 1H), 4.66 (s, 1H), 3.49 (d, J = 5.7 Hz, 2H), 0.88-0.80 (m, 2H), 0.79-0.71 (m, 2H). | 419 |
| | (1-(7-fluoro-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazol-5- | | | |
| | yl)cyclopropyl)methanol | | | |
| Example 442 Method 10 | 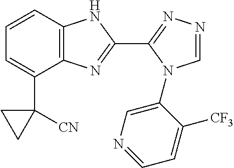 [SMILES: N#CC1(c2cccc3[nH]c(-c4nncn4-c4cnccc4C(F)(F)F)nc23)CC1] | white solid, yield: 6% | ¹H NMR (400 MHz, DMSO-d6) δ 9.19-9.08 (m, 2H), 9.08 (s, 1H), 8.11 (d, J = 5.1 Hz, 1H), 7.46 (dd, J = 6.3, 2.9 Hz, 1H), 7.34-7.20 (m, 2H), 1.38 (dd, J = 7.9, 4.7 Hz, 4H). | 396 |
| | 1-(2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazol-4- | | | |
| | yl)cyclopropane-1- | | | |
| | carbonitrile | | | |
| Example 443 Method 10 | 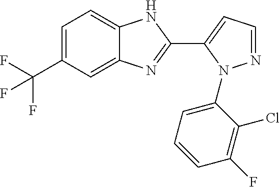 [SMILES: Fc1cccc(-n2nccc2-c2nc3cc(C(F)(F)F)ccc3[nH]2)c1Cl] | white solid, yield: 13.9% | ¹H NMR (400 MHz, Methanol-d₄) δ 7.93 (m, 1H), 7.79 (s, 1H), 7.65 (d, J = 8.4 Hz, 1H), 7.57- 7.39 (m, 4H), 7.16 (m, 1H). | 381.2 |
| | 2-(1-(2-chloro-3- | | | |
| | fluorophenyl)-1H- | | | |
| | pyrazol-5-yl)-5- | | | |
| | (trifluoromethyl)-1H- | | | |
| | benzo[d]imidazole | | | |
| Example 444 Method 20 | 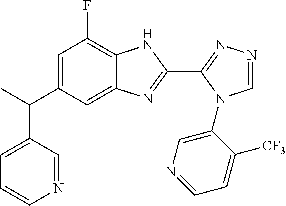 [SMILES: CC(c1cccnc1)c1cc(F)c2[nH]c(-c3nncn3-c3cnccc3C(F)(F)F)nc2c1] | white solid, yield: 81.5% | ¹H NMR (400 MHz, Chloroform-d) δ 13.52 (brs, 1H), 9.08 (d, J = 5.2 Hz, 1H), 8.85 (s, 1H), 8.63 (s, 1H), 8.52- 8.39 (m, 2H), 7.85-7.78 (m, 1H), 7.74 (s, 1H), 7.56-7.49 (m, 1H), 7.25- 7.19 (m, 1H), 6.75 (d, J = 11.6 Hz, 1H), 4.35- 4.19 (m, 1H), 1.72 (d, J = 7.2 Hz, 3H). | 454 |
| | 7-fluoro-5-(1-(pyridin- | | | |
| | 3-yl)ethyl)-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazole | | | |
| Example 445 Method 20 | 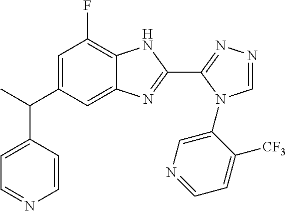 [SMILES: CC(c1ccncc1)c1cc(F)c2[nH]c(-c3nncn3-c3cnccc3C(F)(F)F)nc2c1] | white solid, yield: 4.9% | ¹H NMR (400 MHz, Chloroform-d) δ 12.34 (brs, 1H), 9.08 (d, J = 5.2 Hz, 1H), 8.83 (s, 1H), 8.68-8.52 (m, 2H), 8.47-8.37 (m, 1H), 7.82 (d, J = 5.2 Hz, 1H), 7.54-7.38 (m, 3H), 6.73 (d, J = 11.2 Hz, 1H), 4.45-4.28 (m, 1H), 1.75 (d, J = 7.2 Hz, 3H). | 454 |
| | 7-fluoro-5-(1-(pyridin- | | | |
| | 4-yl)ethyl)-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazole | | | |
| Example 446 Method 8 | 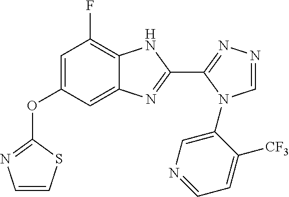 [SMILES: Fc1cc(Oc2nccs2)cc2nc(-c3nncn3-c3cnccc3C(F)(F)F)[nH]c12] | white solid, yield: 13% | ¹H NMR (400 MHz, DMSO-d₆) δ 9.17-9.14 (m, 1H), 9.13-9.10 (m, 2H), 8.09 (d, J = 5.2 Hz, 1H), 7.33-7.29 (m, 1H), 7.29-7.24 (m, 2H), 7.15-7.06 (m, 1H). | 448 |
| | 2-((7-fluoro-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazol-5- | | | |
| | yl)oxy)thiazole | | | |
| Example 447 Method 17 | 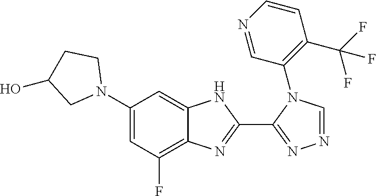 [SMILES: OC1CCN(c2cc(F)c3nc(-c4nncn4-c4cnccc4C(F)(F)F)[nH]c3c2)C1] | white solid, yield: 50.7% | ¹H NMR (400 MHz, Methanol-d₄) δ 9.10- 9.01 (m, 1H), 8.94 (s, 1H), 8.86 (s, 1H), 7.97 (m, 1H), 6.42-6.25 (m, 2H), 4.59-4.49 (m, 2H), 3.53-3.42 (m, 2H), 3.21 (m, 1H), 2.17 (m, 1H), 2.04 (m, 1H). | 434.3 |
| | 1-(4-fluoro-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazol-6- | | | |
| | yl)pyrrolidin-3-ol | | | |
| Example 448 Method 10 | 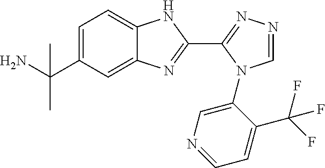 [SMILES: CC(C)(N)c1ccc2[nH]c(-c3nncn3-c3cnccc3C(F)(F)F)nc2c1] | white solid, yield: 48% | ¹H NMR (400 MHz, Chloroform-d) δ 9.09 (br s, 1H), 8.88 (br d, J = 1.7 Hz, 1H), 8.41 (br s, 1H), 8.20-7.71 (m, 2H), 7.67- 7.51 (m, 1H), 7.45- 7.32 (m, 1H), 1.68-1.35 (m, 6H). | 371 |
| | 2-[2-[4-[4- | | | |
| | (trifluoromethyl)-3- | | | |
| | pyridyl]-1,2,4-triazol-3- | | | |
| | yl]-1H-benzimidazol-5- | | | |
| | yl]propan-2-amine | | | |
| Example 449 Method 10 | 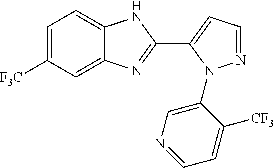 [SMILES: FC(F)(F)c1ccc2[nH]c(-c3ccnn3-c3cnccc3C(F)(F)F)nc2c1] | white solid, yield: 12% | ¹H NMR (400 MHz, Methanol-d₄) δ 9.02 (d, J = 5.1 Hz, 1H), 8.86 (s, 1H), 7.98 (d, J = 2.0 Hz, 1H), 7.94 (d, J = 5.1 Hz, 1H), 7.82-7.45 (m, 3H), 7.24 (d, J = 1.8 Hz, 1H). | 398 |
| | 5-(trifluoromethyl)-2- | | | |
| | (1-(4- | | | |
| | (trifluoromethyl)pyridin- | | | |
| | 3-yl)-1H-pyrazol-5-yl)- | | | |
| | 1H-benzo[d]imidazole | | | |
| Example 450 Method 17 | 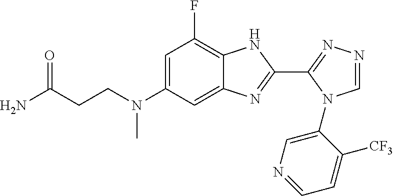 [SMILES: CN(CCC(N)=O)c1cc(F)c2[nH]c(-c3nncn3-c3cnccc3C(F)(F)F)nc2c1] | white solid, yield: 61.9% | ¹H NMR (400 MHz, Methanol-d₄) δ 9.05 (d, J = 5.2 Hz, 1H), 8.94 (s, 1H), 8.87 (s, 1H), 7.96 (d, J = 5.2 Hz, 1H), 6.61- 6.51 (m, 2H), 3.67 (t, J = 7.2 Hz, 2H), 2.97 (s, 3H), 2.46 (t, J = 7.2 Hz, 2H). | 449 |
| | 3-((4-fluoro-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazol-6- | | | |
| | yl)(methyl)amino) | | | |
| | propanamide | | | |
| Example 451 Method 17 | 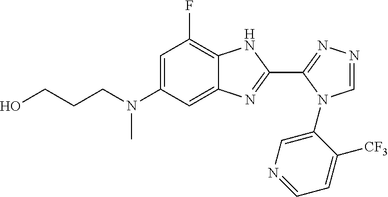 [SMILES: CN(CCCO)c1cc(F)c2[nH]c(-c3nncn3-c3cnccc3C(F)(F)F)nc2c1] | white solid, yield: 40.2% | ¹H NMR (400 MHz, Methanol-d₄) δ 9.05 (d, J = 5.2 Hz, 1H), 8.94 (s, 1H), 8.87 (s, 1H), 7.96 (d, J = 5.2 Hz, 1H), 6.61- 6.51 (m, 2H), 3.61 (t, J = 6.0 Hz, 2H), 3.47 (t, J = 7.2 Hz, 2H), 2.98 (s, 3H), 1.78 (p, J = 6.4 Hz, 2H). | 436 |
| | 3-((4-fluoro-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazol-6- | | | |
| | yl)(methyl)amino) | | | |
| | propan-1-ol | | | |
| Example 452 Method 10 | 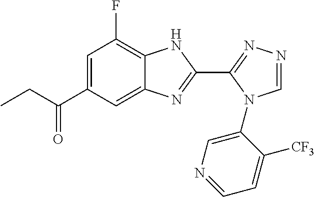 [SMILES: CCC(=O)c1cc(F)c2[nH]c(-c3nncn3-c3cnccc3C(F)(F)F)nc2c1] | white solid, yield: 51.1% | ¹H NMR (400 MHz, Methanol-d₄) δ 9.09 (d, J = 5.2 Hz, 1H), 9.01- 8.92 (m, 2H), 8.09-7.94 (m, 2H), 7.57 (m, 1H), 3.17-2.95 (m, 2H), 1.25- 1.12 (m, 3H). | 405.2 |
| | 1-(4-fluoro-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazol-6- | | | |
| | yl)propan-1-one | | | |
| Example 453 Method 10 | 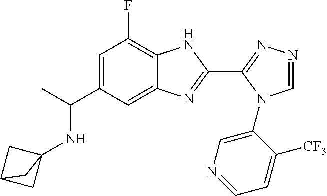 [SMILES: CC(NC12CC(C1)C2)c1cc(F)c2[nH]c(-c3nncn3-c3cnccc3C(F)(F)F)nc2c1] | white solid, yield: 6% | ¹H NMR (400 MHz, Chloroform-d) δ 9.10 (d, J = 5.2 Hz, 1H), 8.86 (d, J = 6.0 Hz, 1H), 8.41 (s, 1H), 7.87-7.77 (m, 1H), 7.66-7.55 (m, 1H), 6.95-6.81 (m, 1H), 4.13-4.01 (m, 1H), 2.38-2.24 (m, 1H), 1.86-1.75 (m, 3H), 1.73-1.58 (m, 4H), 1.52-1.38 (m, 2H). | 458 |
| | N-(1-(7-fluoro-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazol-5- | | | |
| | yl)ethyl)bicyclo[1.1.1] | | | |
| | pentan-1-amine | | | |
| Example 454 Method 10 | 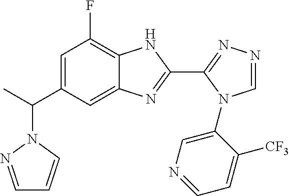 [SMILES: CC(c1cc(F)c2[nH]c(-c3nncn3-c3cnccc3C(F)(F)F)nc2c1)n1cccn1] | white solid, yield: 13% | ¹H NMR (400 MHz, Chloroform-d) δ 13.08 (brs, 1H), 9.09 (d, J = 5.2 Hz, 1H), 8.88-8.83 (m, 1H), 8.43 (s, 1H), 7.82 (d, J = 5.2 Hz, 1H), 7.67-7.44 (m, 3H), 6.76 (d, J = 11.2 Hz, 1H), 6.28 (t, J = 2.0 Hz, 1H), 5.66 (q, J = 7.2 Hz, 1H), 1.96 (d, J = 7.2 Hz, 3H). | 443 |
| | 5-(1-(1H-pyrazol-1- | | | |
| | yl)ethyl)-7-fluoro-2-(4- | | | |
| | (4- | | | |
| | (trifluoromethyl)pyridin- | | | |
| | 3-yl)-4H-1,2,4-triazol- | | | |
| | 3-yl)-1H- | | | |
| | benzo[d]imidazole | | | |
| Example 455 Method 17 | 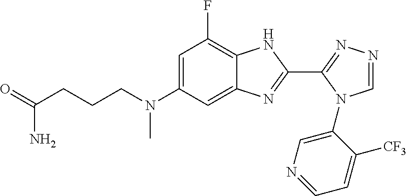 [SMILES: CN(CCCC(N)=O)c1cc(F)c2[nH]c(-c3nncn3-c3cnccc3C(F)(F)F)nc2c1] | white solid, yield: 40.4% | ¹H NMR (400 MHz, Methanol-d₄) δ 9.05 (d, J = 5.2 Hz, 1H), 8.94 (s, 1H), 8.87 (s, 1H), 7.96 (d, J = 5.2 Hz, 1H), 6.59- 6.44 (m, 2H), 3.37 (t, J = 7.2 Hz, 2H), 2.96 (s, 3H), 2.25 (t, J = 7.2 Hz, 2H), 1.88 (p, J = 7.2 Hz, 2H). | 463.2 |
| | 4-((4-fluoro-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazol-6- | | | |
| | yl)(methyl)amino) | | | |
| | butanamide | | | |
| Example 456 Method 10 | 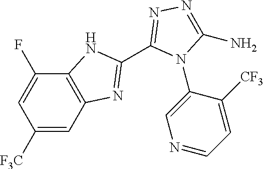 [SMILES: Nc1nnc(-c2nc3cc(C(F)(F)F)cc(F)c3[nH]2)n1-c1cnccc1C(F)(F)F] | white solid, | ¹H NMR (400 MHz, Methanol-d₄) δ = 9.08 (d, J = 5.1 Hz, 1H), 8.94 (s, 1H), 7.98 (d, J = 5.0 Hz, 1H), 7.72-7.50 (m, 1H), 7.37-7.07 (m, 1H). | 432 |
| | 5-(7-fluoro-5- | | | |
| | (trifluoromethyl)-1H- | | | |
| | benzo[d]imidazol-2-yl)- | | | |
| | 4-(4- | | | |
| | (trifluoromethyl)pyridin- | | | |
| | 3-yl)-4H-1,2,4-triazol- | | | |
| | 3-amine | | | |
| Example 457 Method 10 | 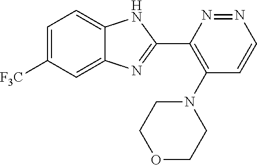 [SMILES: FC(F)(F)c1ccc2[nH]c(-c3nnccc3N3CCOCC3)nc2c1] | white solid, yield: 91% | ¹H NMR (400 MHz, DMSO-d₆) δ 8.99 (d, J = 6.7 Hz, 1H), 8.04 (s, 1H), 7.85 (d, J = 8.5 Hz, 1H), 7.61 (dd, J = 8.5, 1.8 Hz, 1H), 7.55 (d, J = 6.8 Hz, 1H), 3.74 (dd, J = 5.6, 3.6 Hz, 4H), 3.34 (t, J = 4.5 Hz, 4H). | 350 |
| | 4-(3-(5- | | | |
| | (trifluoromethyl)-1H- | | | |
| | benzo[d]imidazol-2- | | | |
| | yl)pyridazin-4- | | | |
| | yl)morpholine | | | |
| Example 458 Method 10 | 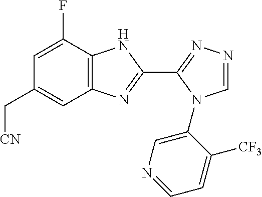 [SMILES: N#CCc1cc(F)c2[nH]c(-c3nncn3-c3cnccc3C(F)(F)F)nc2c1] | white solid, yield: 29% | ¹H NMR (400 MHz, DMSO-d₆) δ 14.06 (s, 1H), 9.16 (s, 1H), 9.12 (d, J = 2.9 Hz, 2H), 8.08 (d, J = 5.1 Hz, 1H), 7.38 (s, 1H), 6.97 (d, J = 11.4 Hz, 1H), 4.14 (s, 2H). | 388 |
| | 7-fluoro-5- | | | |
| | (isocyanomethyl)-2-(4- | | | |
| | (4- | | | |
| | (trifluoromethyl)pyridin- | | | |
| | 3-yl)-4H-1,2,4-triazol- | | | |
| | 3-yl)-1H- | | | |
| | benzo[d]imidazole | | | |
| Example 459 Method 10 | 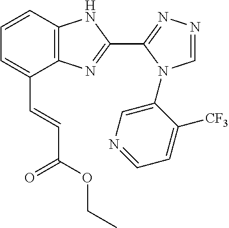 [SMILES: CCOC(=O)/C=C/c1cccc2[nH]c(-c3nncn3-c3cnccc3C(F)(F)F)nc12] | white solid, yield: 22% | ¹H NMR (400 MHz, Chloroform-d) δ 9.16 (d, J = 5.0 Hz, 1H), 8.83 (d, J = 18.3 Hz, 1H), 8.44 (s, 1H), 7.93 (d, J = 5.3 Hz, 1H), 7.81-7.65 (m, 1H), 7.63 (d, J = 15.9 Hz, 1H), 7.35 (m, 2H), 6.40 (d, J = 15.9 Hz, 1H), 4.32 (q, J = 7.1 Hz, 2H), 1.45 (t, J = 7.1 Hz, 3H). | 429 |
| | ethyl (E)-3-(2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazol-4- | | | |
| | yl)acrylate | | | |
| Example 460 Method 20 | 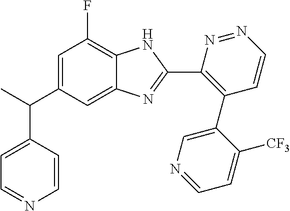 [SMILES: CC(c1ccncc1)c1cc(F)c2[nH]c(-c3nnccc3-c3cnccc3C(F)(F)F)nc2c1] | white solid, yield: 30% | ¹H NMR (400 MHz, Chloroform-d) δ 10.61 (brs, 1H), 9.35 (d, J = 5.2 Hz, 1H), 8.97 (d, J = 5.2 Hz, 1H), 8.62 (s, 1H), 8.53 (d, J = 5.2 Hz, 2H), 8.20 (s, 1H), 7.74 (d, J = 5.2 Hz, 1H), 7.58 (d, J = 5.2 Hz, 1H), 7.29- 7.26 (m, 1H), 7.11 (dd, J = 4.4, 1.2 Hz, 1H), 6.71 (dt, J = 11.2, 1.6 Hz, 1H), 4.22 (q, J = 7.2 | 465 |
| | 7-fluoro-5-(1-(pyridin- | | Hz, 1H), 1.66 (d, J = 7.2 | |
| | 4-yl)ethyl)-2-(4-(4- | | Hz, 3H). | |
| | (trifluoromethyl)pyridin- | | | |
| | 3-yl)pyridazin-3-yl)- | | | |
| | 1H-benzo[d]imidazole | | | |
| Example 461 Method 10 | 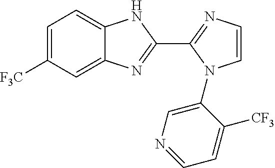 [SMILES: FC(F)(F)c1ccc2[nH]c(-c3nccn3-c3cnccc3C(F)(F)F)nc2c1] | white solid, yield: 38% | ¹H NMR (400 MHz, Methanol-d₄) δ 9.04 (d, J = 5.0 Hz, 1H), 8.92 (s, 1H), 8.15 (s, 1H), 7.96 (d, J = 5.1 Hz, 1H), 7.90 (d, J = 0.6 Hz, 1H), 7.68 (br s, 1H), 7.58- 7.57 (m, 1H), 7.48-7.46 (m, 1H). | 398 |
| | 5-(trifluoromethyl)-2- | | | |
| | (1-(4- | | | |
| | (trifluoromethyl)pyridin- | | | |
| | 3-yl)-1H-imidazol-5- | | | |
| | yl)-1H- | | | |
| | benzo[d]imidazole | | | |
| Example 462 Method 20 | 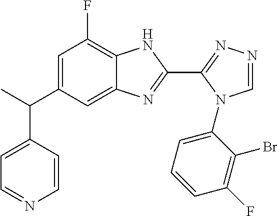 [SMILES: CC(c1ccncc1)c1cc(F)c2[nH]c(-c3nncn3-c3cccc(F)c3Br)nc2c1] | white solid, yield: 3% | ¹H NMR (400 MHz, Chloroform-d) δ 12.18 (brs, 1H), 8.65-8.44 (m, 1H), 8.38 (s, 1H), 8.34 (s, 1H), 7.57-7.49 (m, 1H), 7.49-7.42 (m, 1H), 7.41-7.31 (m, 2H), 7.19 (dd, J = 8.0, 1.6 Hz, 1H), 6.74 (d, J = 11.2 Hz, 1H), 4.32- 4.10 (m, 1H), 1.70 (d, J = 7.2 Hz, 3H). | 482 |
| | 2-(4-(2-bromo-3- | | | |
| | fluorophenyl)-4H-1,2,4- | | | |
| | triazol-3-yl)-7-fluoro-5- | | | |
| | (1-(pyridin-4-yl)ethyl)- | | | |
| | 1H-benzo[d]imidazole | | | |
| Example 463 Method 8 | 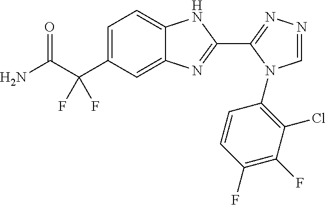 [SMILES: NC(=O)C(F)(F)c1ccc2[nH]c(-c3nncn3-c3ccc(F)c(F)c3Cl)nc2c1] | white solid, yield: 26% | ¹H NMR (400 MHz, Methanol-d₄) δ 8.87 (d, J = 11.4 Hz, 1H), 7.90- 7.46 (m, 5H). | 425 |
| | 2-(2-(4-(2-chloro-3,4- | | | |
| | difluorophenyl)-4H- | | | |
| | 1,2,4-triazol-3-yl)-1H- | | | |
| | benzo[d]imidazol-5-yl)- | | | |
| | 2,2-difluoroacetamide | | | |
| Example 464 Method 21 | 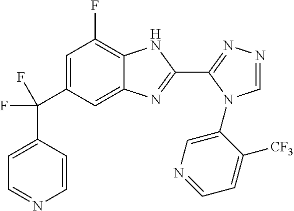 [SMILES: Fc1cc(C(F)(F)c2ccncc2)cc2nc(-c3nncn3-c3cnccc3C(F)(F)F)[nH]c12] | white solid, yield: 58% | ¹H NMR (400 MHz, Methanol-d4) δ 9.06 (d, J = 5.2 Hz, 1H), 8.97 (d, J = 4.4 Hz, 2H), 8.66 (s, 2H), 7.97 (d, J = 5.2 Hz, 1H), 7.61-7.46 (m, 3H), 7.10 (s, 1H). | 476 |
| | 6-(difluoro(pyridin-4- | | | |
| | yl)methyl)-4-fluoro-2- | | | |
| | (4-(4- | | | |
| | (trifluoromethyl)pyridin- | | | |
| | 3-yl)-4H-1,2,4- | | | |
| | triazol-3-yl)-1H- | | | |
| | benzo[d]imidazole | | | |
| Example 465 Method 10 | 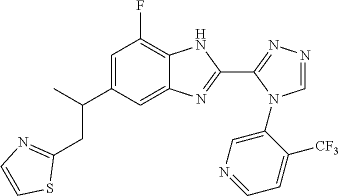 [SMILES: CC(Cc1nccs1)c1cc(F)c2[nH]c(-c3nncn3-c3cnccc3C(F)(F)F)nc2c1] | white solid, | ¹H NMR (400 MHz, Methanol-d⁴) δ 9.11- 9.06 (m, 1H), 8.96 (d, J = 8.6 Hz, 2H), 8.00 (d, J = 5.1 Hz, 1H), 7.64 (d, J = 3.4 Hz, 1H), 7.36 (d, J = 3.4 Hz, 1H), 7.17 (s, 1H), 6.88 (d, J = 11.9 Hz, 1H), 3.37-3.33 (m, 3H), 1.38 (d, J = 6.4 Hz, 3H). | 474 |
| | 2-(2-(7-fluoro-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazol-5- | | | |
| | yl)propyl)thiazole | | | |
| Example 466 Method 10 | 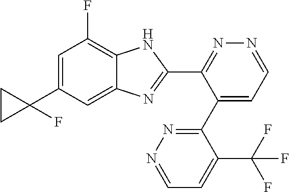 [SMILES: Fc1cc(C2(F)CC2)cc2nc(-c3nnccc3-c3nnccc3C(F)(F)F)[nH]c12] | white solid, yield: 9% | ¹H NMR (400 MHz, Methanol-d₄) δ 9.64 (d, J = 5.5 Hz, 1H), 9.49 (d, J = 5.2 Hz, 1H), 8.23 (d, J = 5.4 Hz, 1H), 7.99 (d, J = 5.2 Hz, 1H), 7.34 (s, 1H), 6.75 (d, J = 11.8 Hz, 1H), 1.15-1.09 (m, 2H), 0.96-0.85 (m, 2H). | 419 |
| | 7-fluoro-5-(1- | | | |
| | fluorocyclopropyl)-2- | | | |
| | (4-(trifluoromethyl)- | | | |
| | [3,4′-bipyridazin]-3′-yl)- | | | |
| | 1H-benzo[d]imidazole | | | |
| Example 467 Method 21 | 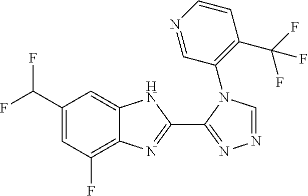 [SMILES: Fc1cc(C(F)F)cc2[nH]c(-c3nncn3-c3cnccc3C(F)(F)F)nc12] | white solid, yield: 45.1% | ¹H NMR (400 MHz, Methanol-d₄) δ 9.08 (d, J = 5.2 Hz, 1H), 8.97 (d, J = 4.7 Hz, 2H), 7.99 (d, J = 5.2 Hz, 1H), 7.55 (s, 1H), 7.08 (s, 1H), 6.83 (t, J = 56.4 Hz, 1H). | 399 |
| | 6-(difluoromethyl)-4- | | | |
| | fluoro-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazole | | | |
| Example 468 Method 21 | 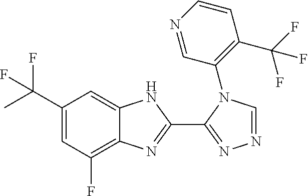 [SMILES: CC(F)(F)c1cc(F)c2nc(-c3nncn3-c3cnccc3C(F)(F)F)[nH]c2c1] | white solid, yield: 29.6% | ¹H NMR (400 MHz, Methanol-d₄) δ 9.08 (d, J = 5.2 Hz, 1H), 8.97 (d, J = 5.6 Hz, 2H), 7.99 (d, J = 5.2 Hz, 1H), 7.52 (s, 1H), 7.11 (s, 1H), 1.94 (t, J = 18.2 Hz, 3H). | 413 |
| | 6-(1,1-difluoroethyl)-4- | | | |
| | fluoro-2-(4-(4- | | | |
| | (trifluoromethyl)pyridin- | | | |
| | 3-yl)-4H-1,2,4-triazol- | | | |
| | 3-yl)-1H- | | | |
| | benzo[d]imidazole | | | |

### Example II. SARM1 (50-724) Enzymatic Assay

An enzymatic assay was performed in a 384-well plate using Dulbecco's phosphate-buffered saline (PBS) as the reaction buffer. Purified SARM1 (50-724) with a final concentration of 2 nM was pre-incubated with a test compound at 100 DMSO final assay concentration for 15 min at room temperature. The reaction was initiated by adding a mixture of 200 M nicotinamide mononucleotide (NMN) as activator and 100 am NAD⁺ as substrate. After 1 h of incubation at room temperature, the reaction was terminated with 10 times volume of 70% acetonitrile, and then centrifuged at 3800 rpm for 10 min. The samples were analyzed using LC-MS/MS after diluted to a proper concentration by 10 mM ammonium acetate (pH 9.75).

SARM1 inhibitory activity of Compounds 1-468 is summarized in Table 2. In Table 2, activity is provided as follows: A: IC₅₀≤100 nM; B: 100 nM<IC₅₀≤500 nM; C: 500 nM<IC₅₀≤1000 nM; D: IC₅₀>1000 nM.

TABLE 2

| Cmpd No. | IC50 | Cmpd No. | IC50 | Cmpd No. | IC50 |
|---|---|---|---|---|---|
| 1 | D | 2 | C | 3 | D |
| 4 | C | 5 | A | 6 | B |
| 7 | D | 8 | D | 9 | D |
| 10 | D | 11 | D | 12 | D |
| 13 | D | 14 | D | 15 | A |
| 16 | C | 17 | D | 18 | B |
| 19 | B | 20 | C | 21 | D |
| 22 | C | 23 | A | 24 | D |
| 25 | D | 26 | D | 27 | D |
| 28 | D | 29 | D | 30 | D |
| 31 | D | 32 | D | 33 | D |
| 34 | D | 35 | B | 36 | B |
| 37 | D | 38 | B | 39 | D |
| 40 | A | 41 | D | 42 | C |
| 43 | C | 44 | B | 45 | A |
| 46 | B | 47 | A | 48 | A |
| 49 | A | 50 | B | 51 | A |
| 52 | D | 53 | A | 54 | B |
| 55 | B | 56 | B | 57 | B |
| 58 | A | 59 | A | 60 | D |
| 61 | C | 62 | D | 63 | B |
| 64 | A | 65 | A | 66 | A |
| 67 | A | 68 | A | 69 | B |
| 70 | D | 71 | B | 72 | B |
| 73 | B | 74 | A | 75 | B |
| 76 | D | 77 | B | 78 | B |
| 79 | B | 80 | B | 81 | B |
| 82 | A | 83 | D | 84 | A |
| 85 | A | 86 | D | 87 | A |
| 88 | A | 89 | B | 90 | A |
| 91 | D | 92 | B | 93 | A |
| 94 | C | 95 | D | 96 | A |
| 97 | D | 98 | D | 99 | B |
| 100 | A | 101 | A | 102 | A |
| 103 | A | 104 | A | 105 | A |
| 106 | A | 107 | A | 108 | B |
| 109 | A | 110 | A | 111 | A |
| 112 | A | 113 | A | 114 | A |
| 115 | A | 116 | B | 117 | B |
| 118 | A | 119 | A | 120 | A |
| 121 | A | 122 | A | 123 | A |
| 124 | A | 125 | A | 126 | A |
| 127 | A | 128 | A | 129 | A |
| 130 | A | 131 | D | 132 | A |
| 133 | A | 134 | A | 135 | A |
| 136 | A | 137 | D | 138 | A |
| 139 | A | 140 | A | 141 | A |
| 142 | D | 143 | A | 144 | A |
| 145 | A | 146 | A | 147 | A |
| 148 | A | 149 | A | 150 | B |
| 151 | A | 152 | A | 153 | B |
| 154 | A | 155 | A | 156 | A |
| 157 | A | 158 | A | 159 | A |
| 160 | A | 161 | A | 162 | A |
| 163 | A | 164 | A | 165 | B |
| 166 | A | 167 | A | 168 | B |
| 169 | D | 170 | A | 171 | A |
| 172 | D | 173 | B | 174 | A |
| 175 | A | 176 | D | 177 | A |
| 178 | B | 179 | B | 180 | A |
| 181 | D | 182 | A | 183 | B |
| 184 | A | 185 | A | 186 | B |
| 187 | A | 188 | B | 189 | A |
| 190 | A | 191 | A | 192 | A |
| 193 | A | 194 | A | 195 | A |
| 196 | D | 197 | A | 198 | D |
| 199 | A | 200 | A | 201 | A |
| 202 | D | 203 | A | 204 | A |
| 205 | A | 206 | A | 207 | A |
| 208 | A | 209 | A | 210 | A |
| 211 | A | 212 | B | 213 | A |
| 214 | D | 215 | A | 216 | B |
| 217 | A | 218 | A | 219 | B |
| 220 | B | 221 | A | 222 | A |
| 223 | A | 224 | A | 225 | A |
| 226 | A | 227 | A | 228 | D |
| 229 | A | 230 | A | 231 | A |
| 232 | A | 233 | A | 234 | A |
| 235 | A | 236 | A | 237 | A |
| 238 | A | 239 | A | 240 | A |
| 241 | A | 242 | A | 243 | A |
| 244 | D | 245 | A | 246 | A |
| 247 | D | 248 | D | 249 | A |
| 250 | A | 251 | A | 252 | A |
| 253 | A | 254 | A | 255 | A |
| 256 | A | 257 | A | 258 | A |
| 259 | A | 260 | A | 261 | A |
| 262 | A | 263 | A | 264 | A |
| 265 | A | 266 | A | 267 | A |
| 268 | D | 269 | D | 270 | A |
| 271 | A | 272 | D | 273 | B |
| 274 | C | 275 | A | 276 | A |
| 277 | A | 278 | A | 279 | D |
| 280 | A | 281 | A | 282 | A |
| 283 | A | 284 | A | 285 | A |
| 286 | A | 287 | A | 288 | D |
| 289 | A | 290 | A | 291 | A |
| 292 | A | 293 | A | 294 | A |
| 295 | A | 296 | B | 297 | A |
| 298 | A | 299 | D | 300 | A |
| 301 | B | 302 | B | 303 | A |
| 304 | C | 305 | A | 306 | A |
| 307 | A | 308 | B | 309 | A |
| 310 | D | 311 | B | 312 | A |
| 313 | C | 314 | A | 315 | A |
| 316 | A | 317 | A | 318 | A |
| 319 | D | 320 | A | 321 | A |
| 322 | B | 323 | A | 324 | B |
| 325 | D | 326 | B | 327 | A |
| 328 | A | 329 | A | 330 | A |
| 331 | A | 332 | A | 333 | C |
| 334 | D | 335 | C | 336 | A |
| 337 | D | 338 | A | 339 | A |
| 340 | B | 341 | B | 342 | C |
| 343 | A | 344 | C | 345 | A |
| 346 | A | 347 | A | 348 | A |
| 349 | A | 350 | D | 351 | D |
| 352 | B | 353 | A | 354 | A |
| 355 | A | 356 | A | 357 | D |
| 358 | B | 359 | A | 360 | A |
| 361 | D | 362 | B | 363 | C |
| 364 | A | 365 | D | 366 | A |
| 367 | D | 368 | D | 369 | D |
| 370 | A | 371 | B | 372 | A |
| 373 | A | 374 | A | 375 | B |
| 376 | A | 377 | B | 378 | D |
| 379 | A | 380 | A | 381 | B |
| 382 | A | 383 | A | 384 | C |
| 385 | A | 386 | A | 387 | A |
| 388 | A | 389 | B | 390 | A |
| 391 | B | 392 | B | 393 | C |
| 394 | A | 395 | D | 396 | A |
| 397 | A | 398 | A | 399 | A |
| 400 | A | 401 | A | 402 | B |
| 403 | A | 40 | A | 405 | B |
| 406 | B | 407 | A | 40 | A |
| 409 | A | 410 | A | 411 | A |
| 412 | A | 413 | A | 414 | A |
| 415 | A | 416 | A | 417 | A |
| 418 | A | 419 | B | 420 | A |
| 421 | A | 422 | A | 423 | A |
| 424 | D | 425 | A | 426 | A |
| 427 | A | 428 | A | 429 | A |
| 430 | A | 431 | A | 432 | A |
| 433 | A | 434 | A | 435 | A |
| 436 | A | 437 | D | 438 | D |
| 439 | B | 440 | A | 441 | A |
| 442 | B | 443 | B | 444 | A |
| 445 | A | 446 | B | 447 | C |
| 448 | B | 449 | A | 450 | A |
| 451 | A | 452 | A | 453 | A |
| 454 | A | 455 | A | 456 | A |
| 457 | C | 458 | A | 459 | C |
| 460 | A | 461 | A | 462 | A |
| 463 | A | 464 | A | 465 | A |
| 466 | A | 467 | A | 468 | A |

All publications, including but not limited to disclosures and disclosure applications, cited in this specification are herein incorporated by reference as though fully set forth. If certain content of a publication cited herein contradicts or is inconsistent with the present disclosure, the present disclosure controls.

One skilled in the art will readily recognize from the disclosure and claims that various changes, modifications, and variations can be made therein without departing from the spirit and scope of the disclosure as defined in the following claims.

1. A compound of the following structural Formula 1: 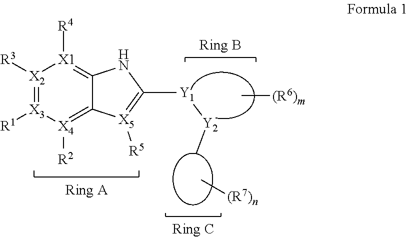
a tautomer thereof, a solvate or stereoisomer of the compound or the tautomer, or a pharmaceutically acceptable salt of the foregoing, wherein:
X₁, X₂, X₃, X₄, and X₅ are each independently C or N;
Y₁ is C or N, Y₂ is C or N, and Y₁ and Y₂ are two adjacent ring atoms on Ring B;
Ring B is phenyl, 5- to 6-membered heteroaryl, 3-6 membered cycloalkyl, or 4- to 6-membered heterocyclyl, wherein the 5- to 6-membered heteroaryl or 4- to 7-membered heterocyclyl of Ring B contains 1 to 4 heteroatoms selected from N, O, and S;
Ring C is phenyl, 3- to 10-membered cycloalkyl, 4- to 10-membered heterocyclyl, 5- to 6-membered heteroaryl, or 9- to 10-membered heteroaryl, wherein the 4- to 10-membered heterocyclyl, 5- to 6-membered heteroaryl, or 9- to 10-membered heteroaryl of Ring C contains 1 to 3 heteroatoms selected from N, S, and O;
R¹ is selected from H, halogen, C₁-C₃ alkyl, C₁-C₈ alkenyl, C₁-C₈ alkynyl, —CN, —OH, —COOH, —C(═O)NH₂, —OR^{m}, —S(═O)ₚ(C₁-C₄alkyl), —NR^{m}Rⁿ, —C(═O)Rⁿ, —C(═O)OR^{m}, —C(═O)NR^{m}Rⁿ, —P(═O)R^{m}Rⁿ, —SF₅,
5- to 6-membered heteroaryl containing 1 to 3 heteroatoms independently selected from N, O, and S,
3- to 10-membered heterocyclyl containing 1 to 2 heteroatoms independently selected from N, O, and S, and
3- to 10-membered cycloalkyl, wherein:
the C₁-C₈ alkyl, C₁-C₈ alkenyl, or C₁-C₈ alkynyl of R¹ is optionally substituted with 1-3 groups selected from halogen, —OH, —OR^{m}, —CN, —NH₂,
NR^{m}Rⁿ, —C(═O)OCH₃, —O(C₁-C₆ alkyl), —COOH, —C(═O)NH₂, phenyl, 5 to 6-membered heteroaryl, 3 to 6-heterocyclyl, and 3- to 6-membered cycloalkyl (optionally substituted with 1-3 groups selected from OH and halogen),
the 5- to 6-membered heteroaryl of R¹ is optionally substituted by 1 to 3 groups selected from D, halogen, —OH, —CN, —COOH, —(C₁-C₆ alkyl)OH, —C(═O)O(C₁-C₆ alkyl), ═O, —NH₂, —C(═O)NR^{m}Rⁿ, 5- to 6-membered heteroaryl, —OR^{m}, R^{m}, C₁-C₆ alkyl (optionally substituted with 1-3 groups selected from halogen, —C(═O)NH₂, R^{m}, and OR^{m}),
the 3- to 10-membered heterocyclyl of R¹ is optionally substituted by 1 to 3 groups selected from D, halogen, —OH, —CN, —COOH, —(C₁-C₆ alkyl)OH, —C(═O)O(C₁-C₆ alkyl), ═O, —NH₂, —C(═O)NR^{m}Rⁿ, 5- to 6-membered heteroaryl, —OR^{m}, R^{m}, C₁-C₆ alkyl (optionally substituted with 1-3 groups selected from halogen, —C(═O)NH₂, R^{m}, and OR^{m}), and
the 3- to 10-membered cycloalkyl of R¹ is optionally substituted by 1 to 3 groups selected from D, halogen, —OH, —CN, —COOH, —(C₁-C₆ alkyl)OH, —C(═O)O(C₁-C₆ alkyl), ═O, —NH₂, —C(═O)NR^{m}Rⁿ, 5- to 6-membered heteroaryl, —OR^{m}, R^{m}, C₁-C₆ alkyl (optionally substituted with 1-3 groups selected from halogen, —C(═O)NH₂, R^{m}, and OR^{m}), and
wherein R^{m} and Rⁿ, for each occurrence, are each independently selected from H, C₁-C₆ alkyl, —S(═O)ₚ(C₁-C₄ alkyl), phenyl, 3- to 8-membered cycloalkyl, 4- to 6-membered heterocyclyl, and 5- to 6-membered heteroaryl, wherein the C₁-C₆ alkyl of R^{m} is optionally substituted with 1-3 groups selected from D, —C(═O)NH₂, —OH, —OMe, —S(═O)₂CH₃, and halogen;
R² is selected from H, halogen, C₁-C₆ alkyl, C₁-C₆ alkenyl, —OH, —O(C₁-C₆ alkyl), —O(C₁-C₆ alkyl)O(C₁-C₆ alkyl), —C(═O)NH₂, —S(═O)ₚ(C₁-C₄alkyl), —CN, 3- to 6-membered cycloalkyl, phenyl, 5- to 6-membered heteroaryl, and 4- to 10-membered heterocyclyl (containing 1 to 3 heteroatoms independently selected from S, O, and N), wherein:
the C₁-C₆ alkyl or C₁-C₆ alkenyl of R² is optionally substituted with 1-3 groups selected from halogen, CN, and —C(═O)O(C₁-C₆ alkyl),
the 3- to 5-membered cycloalkyl of R² is optionally substituted with 1-3 groups selected from OH, CN, and halogen,
the C₁-C₆ alkyl of the —O(C₁-C₆ alkyl) of R² is optionally substituted with 1-3 groups selected from halogen and CN, and
the 3- to 10-membered heterocyclyl of R² is optionally substituted with 1-3 groups selected from OH, CN, and halogen; or
R¹ and R² join to form 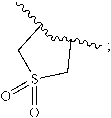
R³ and R⁴ are each independently selected from H, halogen, C₁-C₆ alkyl (optionally substituted with 1-3 groups selected from OH and halogen), and —O(C₁-C₆ alkyl);
R⁵ is selected from absent, H, —CN, halogen, —C(═O)NH₂, —S(═O)ₚ(C₁-C₄ alkyl), —OR^{p}, phenyl, 5- to 6-membered heteroaryl, 4- to 6-membered heterocyclyl, 3- to 8-membered cycloalkyl, and C₁-C₆ alkyl, wherein:
the C₁-C₆ alkyl of R⁵ is optionally substituted with 1 to 3 groups selected from OH, —NHR^{p}, —OR^{p}, and —S(═O)ₚ(C₁-C₄ alkyl),
the 4- to 6-membered heterocyclyl of R⁵ is optionally substituted with 1 to 3 groups selected from C₁-C₃ alkyl, CN, halogen, and ═O,
the 3- to 8-membered cycloalkyl of R⁵ is optionally substituted with 1 to 3 groups selected from C₁-C₃ alkyl, CN, and halogen, wherein:
R^{p} is selected from C₁-C₆ alkyl, 3- to 6-membered cycloalkyl, and 5- to 6-membered heteroaryl, wherein the C₁-C₆ alkyl, 3- to 6-membered cycloalkyl, or 5- to 6-membered heteroaryl of R^{p} is optionally substituted with 1 to 3 groups selected from CN, OH, and halogen;
R⁶, for each occurrence, is independently selected from D, halogen, —CN, ═O, OR⁵, —SH, —S(C₁-C₄alkyl), —S(═O)ₚR^{t}, —C(═O)NR^{t}R^{o}, —NR^{t}R^{o}, 4- to 6-membered heterocyclyl, and C₁-C₆ alkyl, wherein:
the C₁-C₆ alkyl of R⁵ is optionally substituted with 1 to 3 groups selected from halogen, —OR^{s}, ═O, —S(═O)ₚR^{t}, —NHS(═O)ₚR^{t}, —S(═O)(═NH)R^{t}, 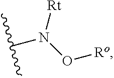
NHS(═O)ₚ(C₁-C₄alkyl), —CN, —C(═O)NR^{t}R^{o}, —NR^{t}R^{o}, halogen, 5- to 6-membered heteroaryl, 3- to 6-membered cycloalkyl (optionally substituted with 1 to 3 groups selected from halogen, OH, and R^{t}), and 4- to 10-membered heterocyclyl optionally substituted with 1 to 3 groups selected from halogen, OH, and R^{t}, wherein:
the 4- to 8-membered heterocyclyl of the C₁-C₆ alkyl of R⁵ is optionally substituted with 1 to 3 groups selected from halogen, OH, C₁-C₃ alkyl, and =0;
R^{s} is selected from H, C₁-C₆ alkyl, 4- to 6-membered heterocyclyl, and 3- to 6-membered cycloalkyl, wherein:
the C₁-C₆ alkyl of R^{s} is optionally substituted with 1-3 groups selected from —OH, —OMe, and halogen and
the 3- to 6-membered cycloalkyl of R^{s} is optionally substituted with —OH or —OMe;
R^{t} and R^{o}, for each occurrence, are each independently selected from H, C₁-C₆ alkyl, 5- to 6-membered heteroaryl, 4- to 6-membered heterocyclyl, and 3- to 5-membered cycloalkyl, wherein the C₁-C₆ alkyl of R^{t} and R^{o} is optionally substituted with 1-3 groups selected from D, halogen, —OH, CN, C(═O)NH₂, —O(C₁-C₃ alkyl), and —S(═O)₂CH₃;
R⁷, for each occurrence, is independently selected from D, halogen, —OR^{a}, —CN, —CONH₂, —C(═O)NR^{b}R^{c}, NR^{b}R^{c}, —C(═O)OR^{b}, ═O, ═S, —P(═O)₂R^{b}R^{c}, —S(═O)ₚ(C₁-C₄alkyl), —O(C₁-C₆ alkyl), C₁-C₆ alkyl, C₁-C₆ alkenyl, C₁-C₆ alkynyl, 3- to 6-membered cycloalkyl, 4- to 6-membered heterocyclyl, and 5- to 6-membered heteroaryl, wherein:
the C₁-C₆ alkyl, C₁-C₆ alkenyl, or C₁-C₆ alkynyl of R⁷ is optionally substituted with 1 to 3 groups selected from halogen, —OH, CN, —S(═O)ₚ(C₁-C₄alkyl), —C(═O)₂NH₂, and 3- to 6-membered heterocyclyl,
the 4- to 6-membered heterocyclyl of R⁷ is optionally substituted with 1 to 3 groups selected from ═O, halogen, and R^{b},
R^{a} is selected from H, C₁-C₈ alkyl, 3- to 6-membered cycloalkyl, 4- to 6-membered heterocyclyl, phenyl, and 5- to 6-membered heteroaryl, wherein the C₁-C₈ alkyl of R^{a} is optionally substituted with 1 to 4 groups selected from D, halogen, OH, CN, —S(═O)ₚ(C₁-C₄alkyl), —C(═O)NH₂, 3- to 6-membered cycloalkyl, 4- to 6-membered heterocyclyl, and 5- to 6-membered heteroaryl,
R^{b} and R^{c}, for each occurrence, are each independently selected from H, C₁-C₈ alkyl, 4- to 6-membered heterocyclyl, phenyl, 5- to 6-membered heteroaryl, and 3- to 6-membered cycloalkyl, wherein the C₁-C₈ alkyl of R^{b} and R^{c} is optionally substituted with 1 to 3 groups selected from D, halogen, OH, —C(═O)NH₂, CN, —OCH₃, and —S(═O)₂CH₃;
m is an integer selected from 0, 1, and 2;
n is an integer selected from 0, 1, 2, 3, and 4; and
p is an integer selected from 0, 1, and 2.

2. The compound, tautomer, solvate, stereoisomer, or pharmaceutically acceptable salt of claim 1, wherein X₁, X₂, X₃, and X₄ are C.

3. The compound of claim 1, wherein the compound has the following structural Formula 2: 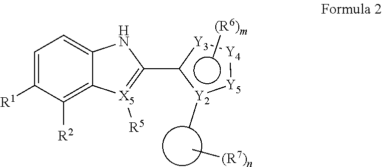
a tautomer thereof, a solvate or stereoisomer of the compound or the tautomer, or a pharmaceutically acceptable salt of the foregoing, wherein Y₂, Y₃, and Y₄ are each independently selected from N and C, at least one of Y₂, Y₃, and Y₄ is N, and Y₅ is selected from S and C.

4. The compound of claim 1, wherein the compound has the following structural Formula 3-1, 3-2, or 3-3: 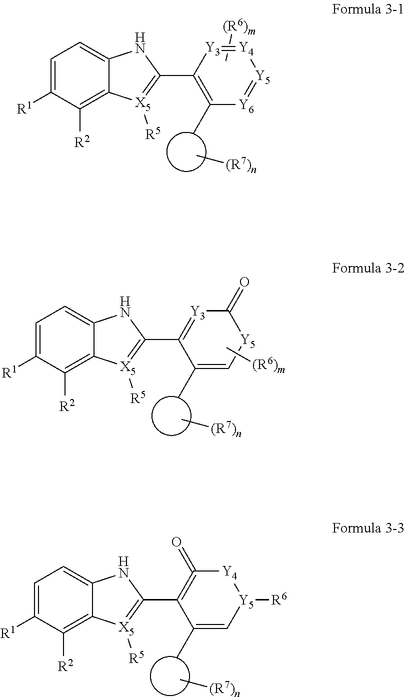
a tautomer thereof, a solvate or stereoisomer of the compound or the tautomer, or a pharmaceutically acceptable salt of the foregoing, wherein Y₃, Y₄, Y₅, and Y₆ are each independently selected from N and C and at least one of Y₃, Y₄, Y₅, and Y₆ is N.

5. The compound of claim 1, wherein the compound has the following structural Formula 4: 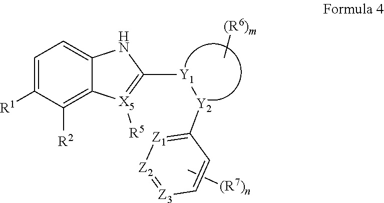
a tautomer thereof, a solvate or stereoisomer of the compound or the tautomer, or a pharmaceutically acceptable salt of the foregoing, wherein Z₁, Z₂, and Z₃ are each independently selected from N and C.

6. The compound of claim 1, wherein the compound has the following structural Formula 5: 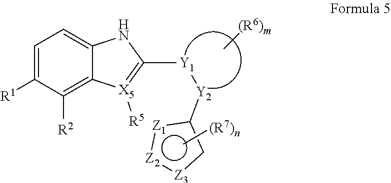
a tautomer thereof, a solvate or stereoisomer of the compound or the tautomer, or a pharmaceutically acceptable salt of the foregoing, wherein Z₁, Z₂, and Z₃ are each independently selected from N, S, and C.

7. The compound of claim 1, wherein the compound has the following structural Formula 6-1 or 6-2: 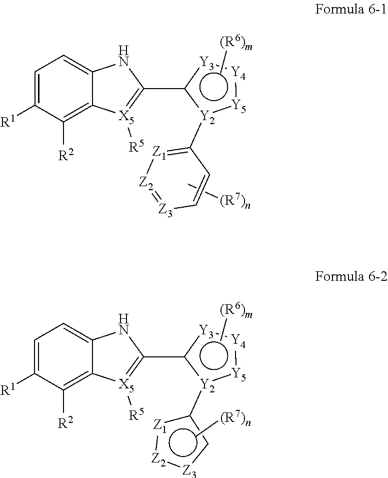
a tautomer thereof, a solvate or stereoisomer of the compound or the tautomer, or a pharmaceutically acceptable salt of the foregoing, wherein Y₃ and Y₄ and Y₅ are each independently selected from N, S, O, and C, Y₂ is selected from N and C, the Z₁, Z₂, and Z₃ of Formula 6-1 are each independently selected from N and C, and the Z₁, Z₂, and Z₃ of Formula 6-2 are each independently selected from N, S, and C.

8. The compound of claim 1, wherein the compound has the following structural Formula 7-1 or 7-2: 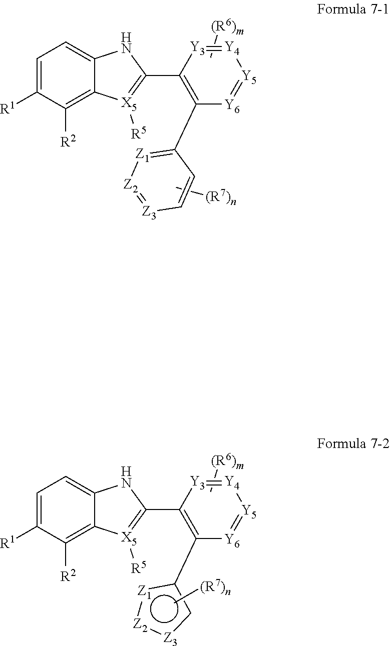
a tautomer thereof, a solvate or stereoisomer of the compound or the tautomer, or a pharmaceutically acceptable salt of the foregoing, wherein Y₃, Y₄, Y₅, and Y₆ are each independently selected from N and C and at least one of Y₃, Y₄, Y₅, and Y₆ is N, the Z₁, Z₂, and Z₃ of Formula 7-1 are each independently selected from N and C, and the Z₁, Z₂, and Z₃ of Formula 7-2 are each independently selected from N, S, and C.

9. The compound of claim 1, wherein the compound has the following structural Formula 8-1, 8-2, 8-3, 8-4, 8-5, or 8-6: 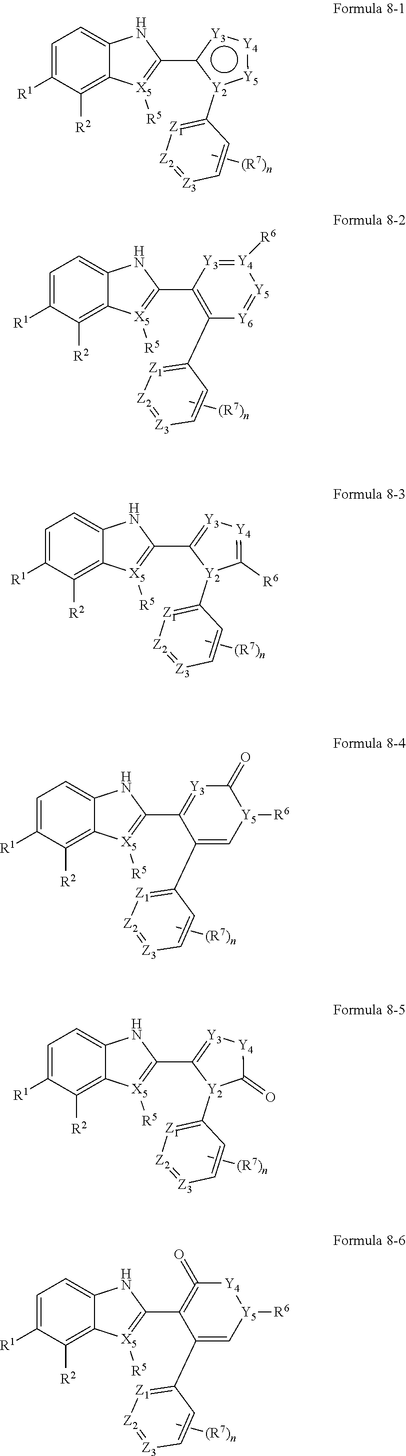
a tautomer thereof, a solvate or stereoisomer of the compound or the tautomer, or a pharmaceutically acceptable salt of the foregoing, wherein:
Y₃, Y₄, Y₅, and Y₆ of Formula 8-2, Formula 8-4, and Formula 8-6 are each independently selected from N and C and at least one of Y₃, Y₄, Y₅, and Y₆ is N,
Y₂, Y₃, and Y₄ of Formula 8-1, Formula 8-3, and Formula 8-5 are each independently selected from N and C, at least one of Y₂, Y₃, and Y₄ is N, Y₅ of Formula 8-1 is selected from N, S, and C;
Z₁, Z₂, and Z₃ are each independently selected from N and C.

10. The compound of claim 1, wherein the compound has the following structural Formula 9-1, 9-2, 9-3, or 9-4: 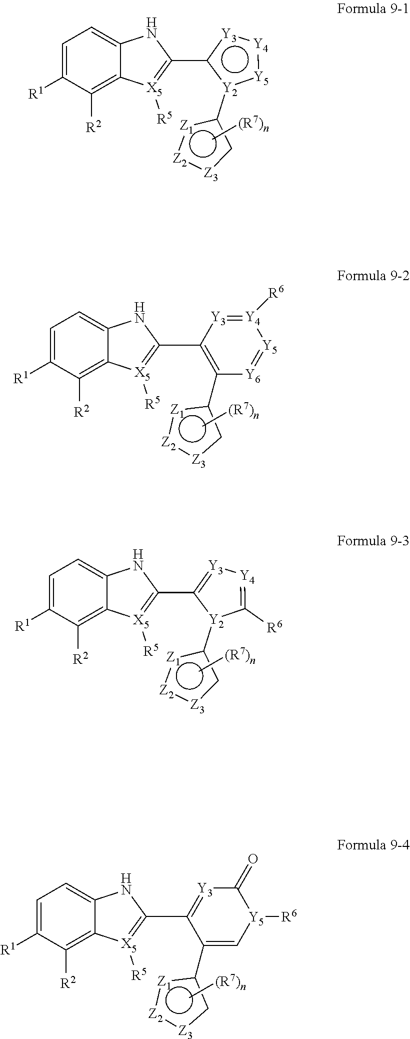
a tautomer thereof, a solvate or stereoisomer of the compound or the tautomer, or a pharmaceutically acceptable salt of the foregoing, wherein:
Y₃, Y₄, Y₅, and Y₆ of Formula 9-2 and Formula 9-4 are each independently selected from N and C and at least one of Y₃, Y₄, Y₅, and Y₆ is N,
Y₂, Y₃, and Y₄ of Formula 9-1 and Formula 9-3 are each independently selected from N and C and at least one of Y₂, Y₃, and Y₄ is N, Y₅ of Formula 9-1 is selected from S and C;
Z₁ and Z₂ are each independently selected from N and S, Z₃ is selected from N and C, and at least one of Z₁, Z₂, and Z₃ is a heteroatom.

11. The compound of claim 1, wherein the compound has the following structural Formula 10-1, 10-2, 10-3, 10-4, 10-5, or 10-6: 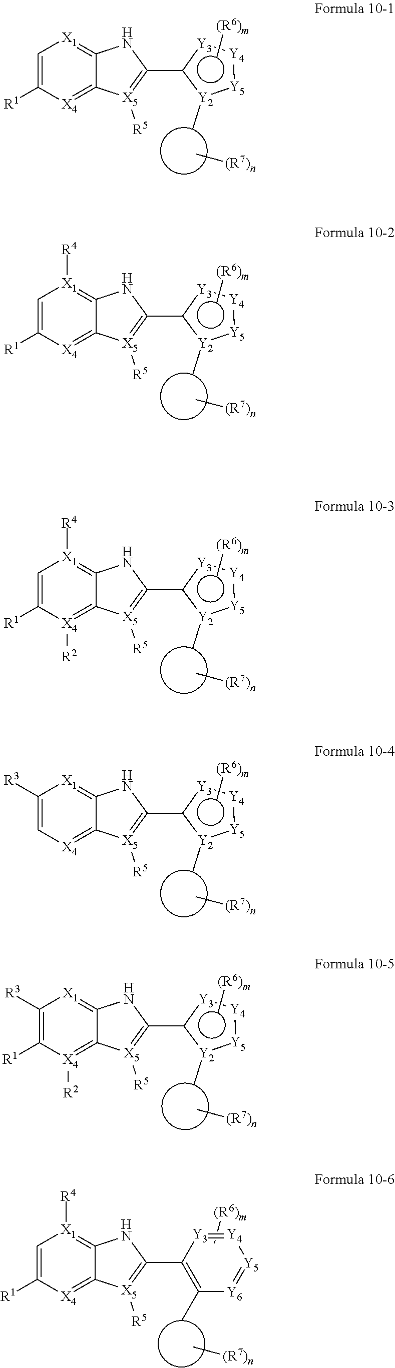
a tautomer thereof, a solvate or stereoisomer of the compound or the tautomer, or a pharmaceutically acceptable salt of the foregoing, wherein:
Y₂, Y₃, and Y₄ of Formula 10-1 to 10-5 are each independently selected from N and C, Y₅ is selected from N, S, and C, and at least one of Y₂, Y₃, Y₄, and Y₅ is a heteroatom;
Y₃, Y₄, Y₅, and Y₆ of Formula 10-6 are each independently selected from N and C, and at least one of Y₃, Y₄, Y₅, and Y₆ is a heteroatom.

12. The compound of claim 1, wherein the compound has the following structural Formula 11-1, 11-2, 11-3, 11-4, 11-5, 11-6, or 11-7: 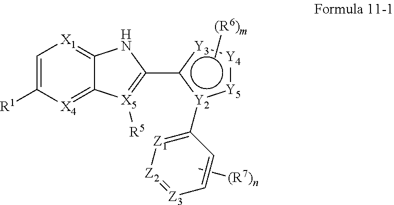 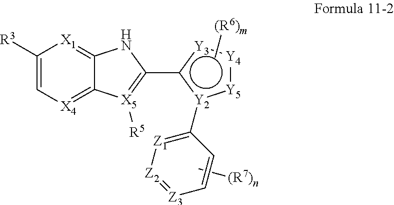 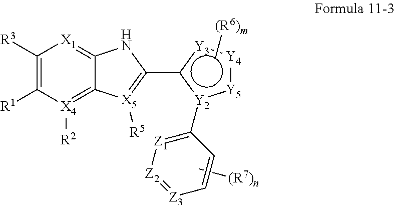 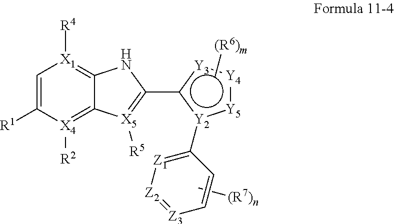 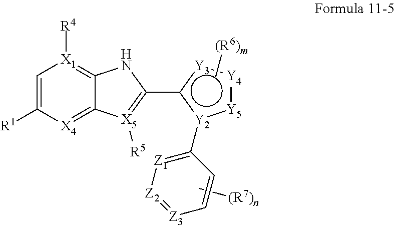 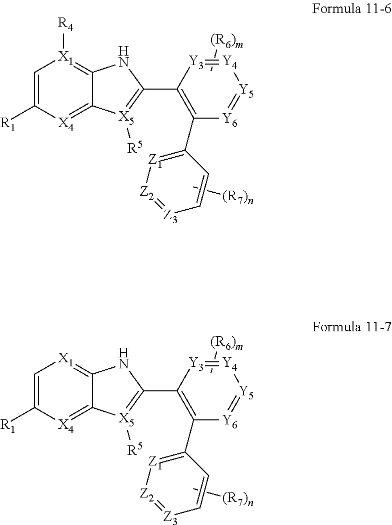
a tautomer thereof, a solvate or stereoisomer of the compound or the tautomer, or a pharmaceutically acceptable salt of the foregoing, wherein:
in Formula 11-1 to 11-5, Y₂, Y₃, and Y₄ are each independently selected from N and C, Y₅ is selected from N, S, and C, at least one of Y₂, Y₃, Y₄, and Y₅ is a heteroatom, Z₁, Z₂, and Z₃ are each independently selected from N and C;
in Formula 11-6 to 11-7, Y₃, Y₄, Y₅, and Y₆ are each independently selected from N and C, at least one of Y₃, Y₄, Y₅, and Y₆ is a heteroatom, Z₁, Z₂, and Z₃ are each independently selected from N and C.

13. The compound of claim 1, wherein the compound has the following structural Formula 12-1, 12-2, 12-3, 12-4, 12-5, 12-6, 12-7, or 12-8: 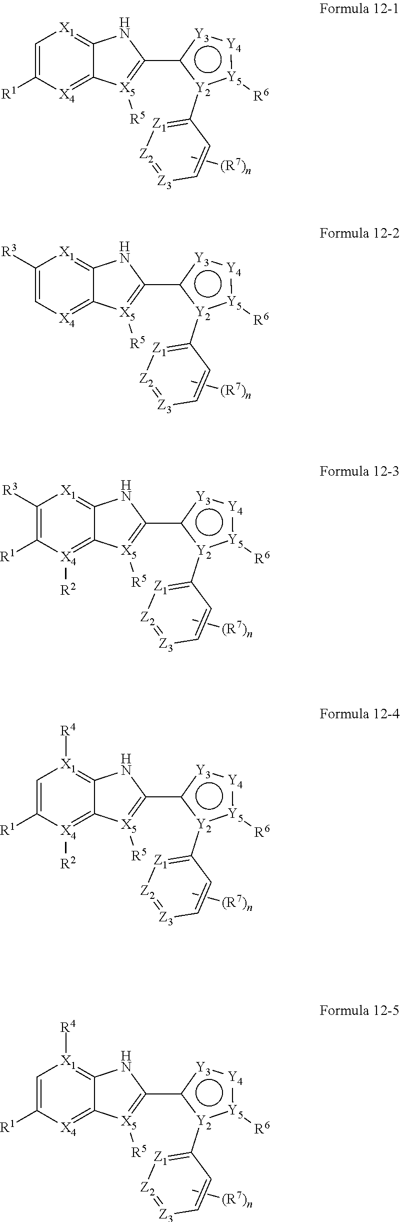 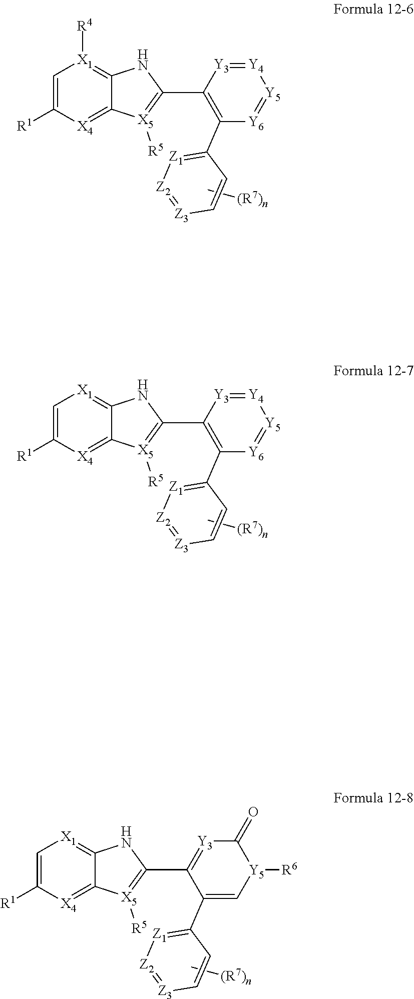
a tautomer thereof, a solvate or stereoisomer of the compound or the tautomer, or a pharmaceutically acceptable salt of the foregoing, wherein:
in Formula 12-1 to 12-5, Y₂ and Y₅ are each independently selected from N and C, Y₃ and Y₄ are each independently selected from N, S, and C, at least one of Y₂, Y₃, Y₄, and Y₅ is a heteroatom, and Z₁, Z₂, and Z₃ are each independently selected from N and C;
in Formula 12-6 to 12-8, Y₃, Y₄, Y₅, and Y₆ are each independently selected from N and C, at least one of Y₃, Y₄, Y₅, and Y₆ is a heteroatom, and Z₁, Z₂, and Z₃ are each independently selected from N and C.

14. The compound, tautomer, solvate, stereoisomer, or pharmaceutically acceptable salt of claim 1, wherein: 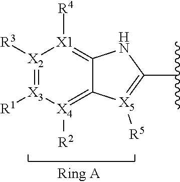
is selected from: 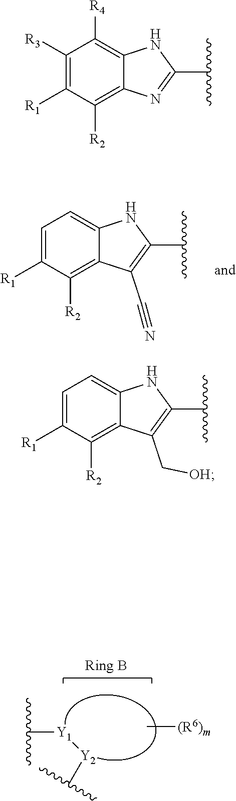
is selected from: 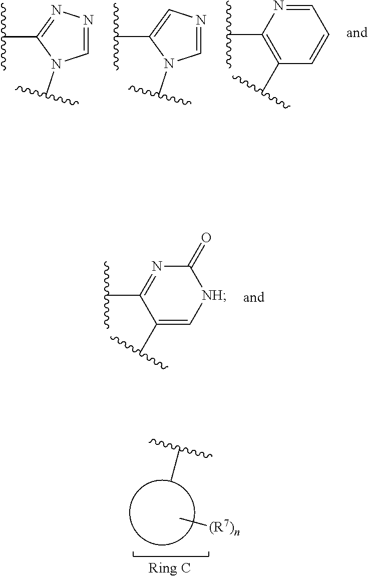
is selected from:

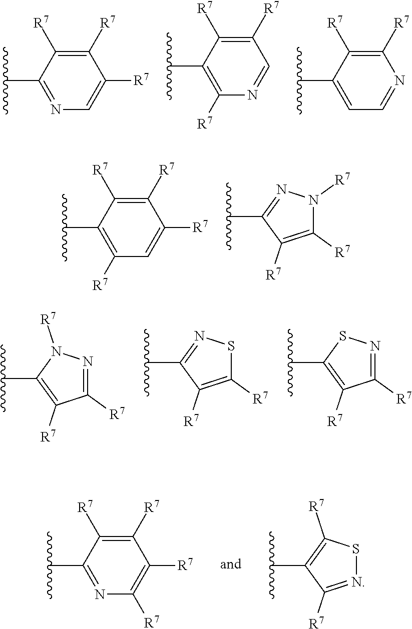

15. The compound, tautomer, solvate, stereoisomer, or pharmaceutically acceptable salt of claim 14, wherein 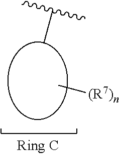
is selected from: 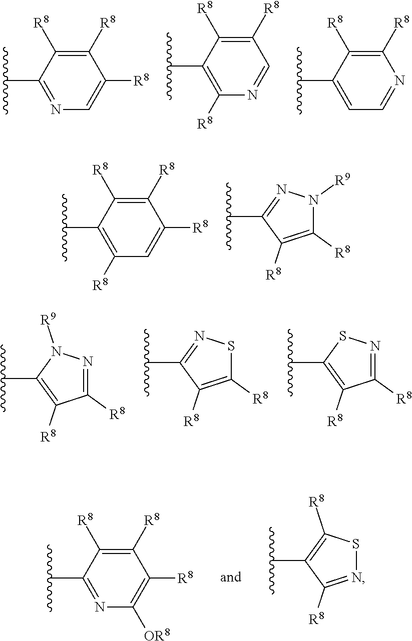
wherein:
R⁸ is selected from: H, F, Cl, Me, CHF₂, CF₃, CN, SO₂Me, SMe, CH₂CF₃, CH₂SO₂Me, 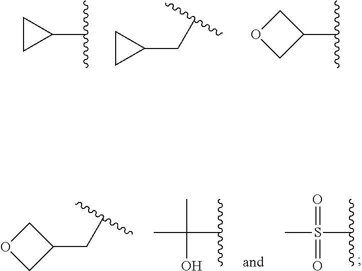
R⁹ is selected from: Me, CF₃, CHF₂, CH₂CF₃, Acetyl (—C(═O)CH₃), SO₂Me,

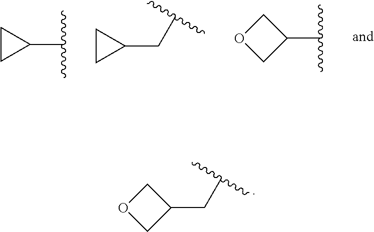

16. The compound, tautomer, solvate, stereoisomer, or pharmaceutically acceptable salt of claim 1, wherein: 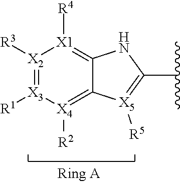
is selected from: 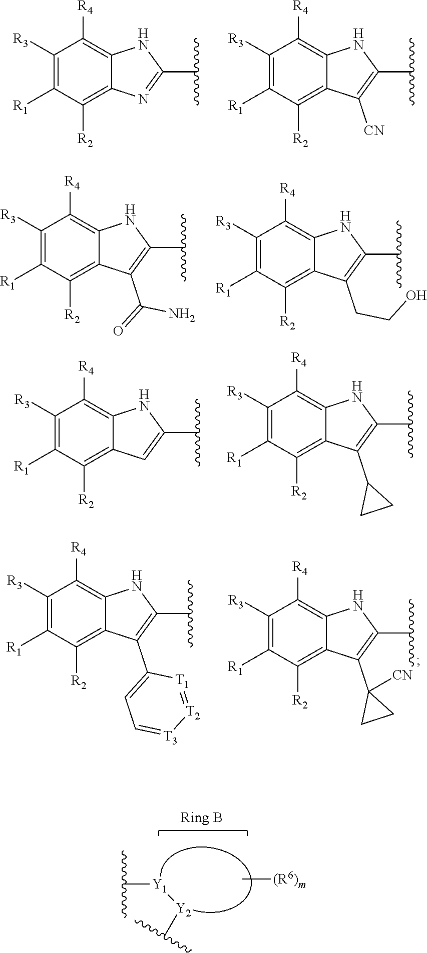
is selected from: 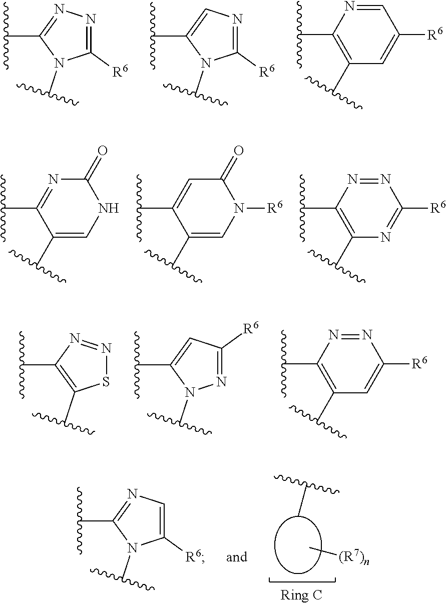
is selected from: 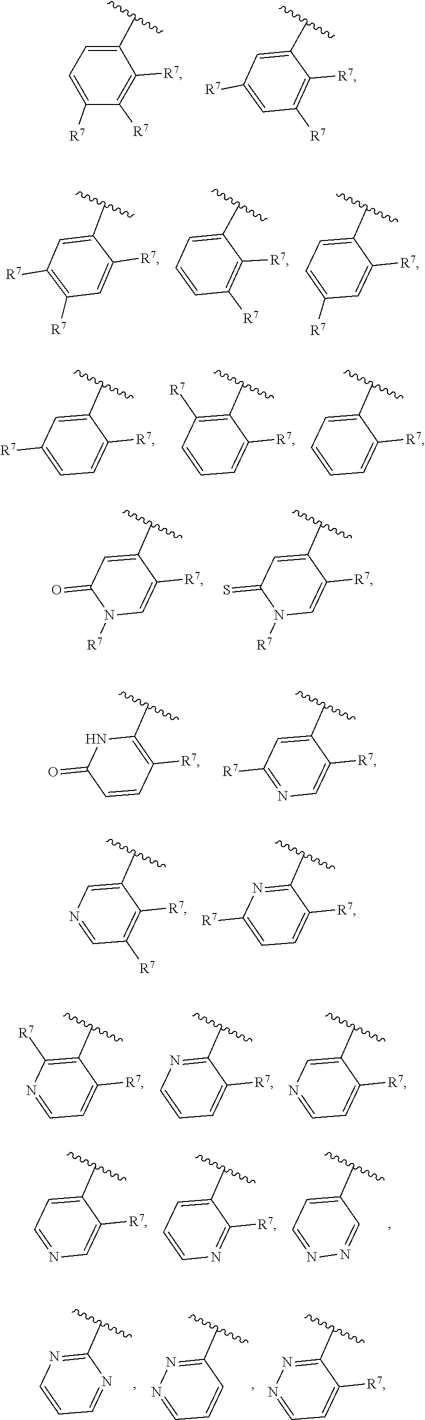 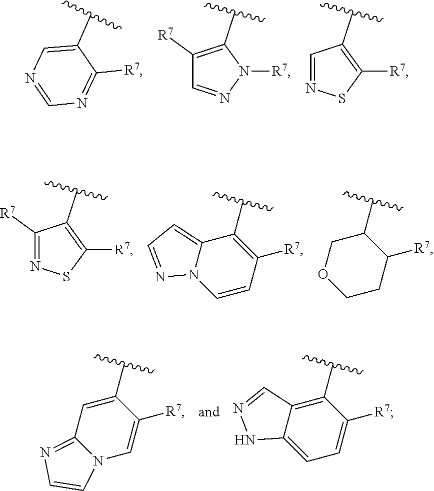
wherein T₁, T₂, and T₃ are each independently selected from N and C.

17. The compound, tautomer, solvate, stereoisomer, or pharmaceutically acceptable salt of claim 1, wherein Ring A is selected from: 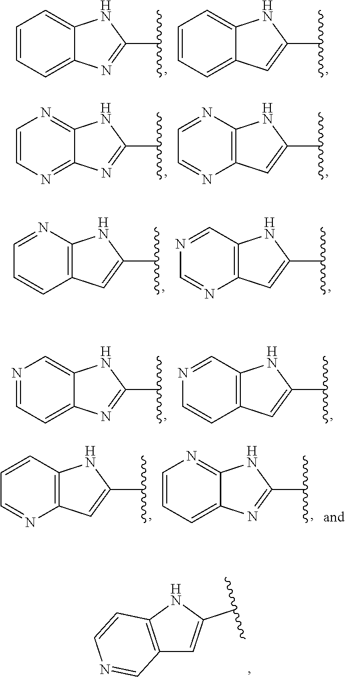
wherein Ring A is substituted with R¹, R², R³, R⁴, and R⁵.

18. The compound, tautomer, solvate, stereoisomer, or pharmaceutically acceptable salt of claim 1, wherein 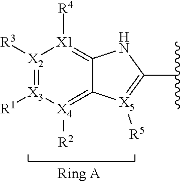
is selected from: 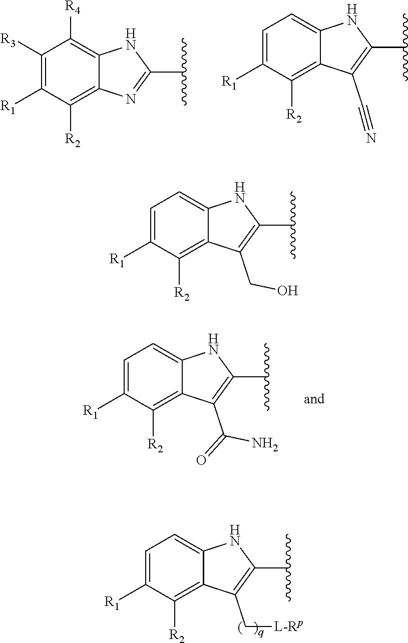
wherein L is —NH— or —O—, q is 1, 2, or 3, and R^{p} is selected from C₁-C₄ alkyl, 3- to 6-membered cycloalkyl, and 5- to 7-membered heteroaryl.

19. The compound, tautomer, solvate, stereoisomer, or pharmaceutically acceptable salt of claim 1, wherein 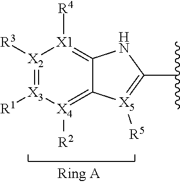
is selected from:

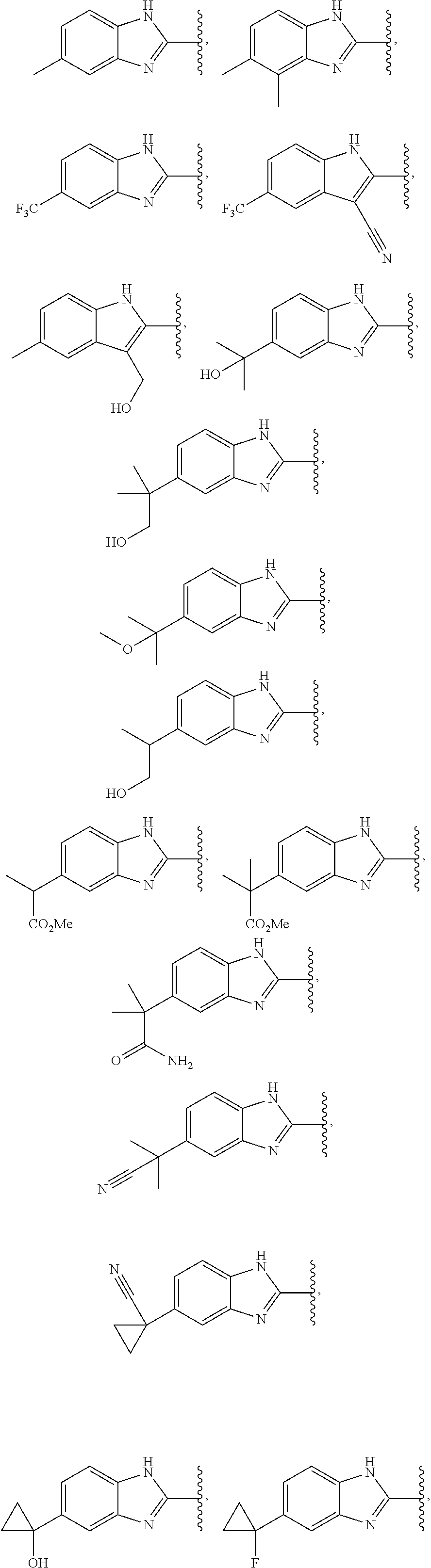

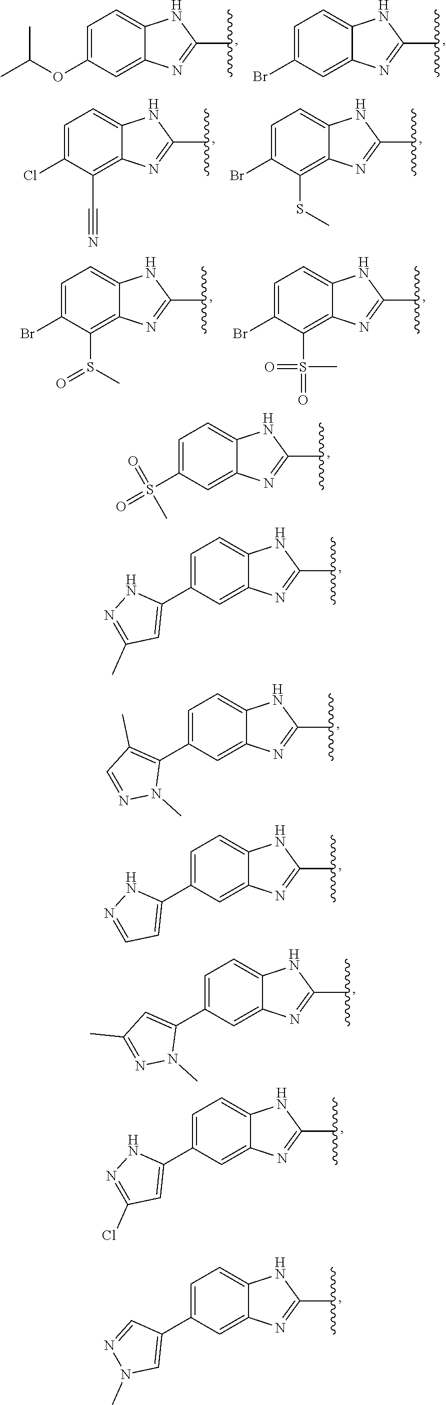

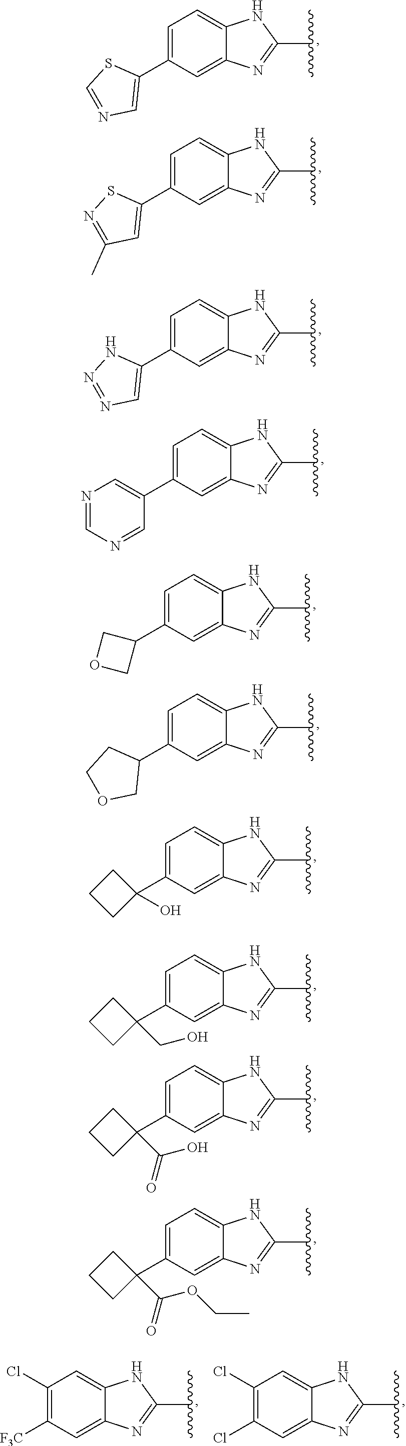

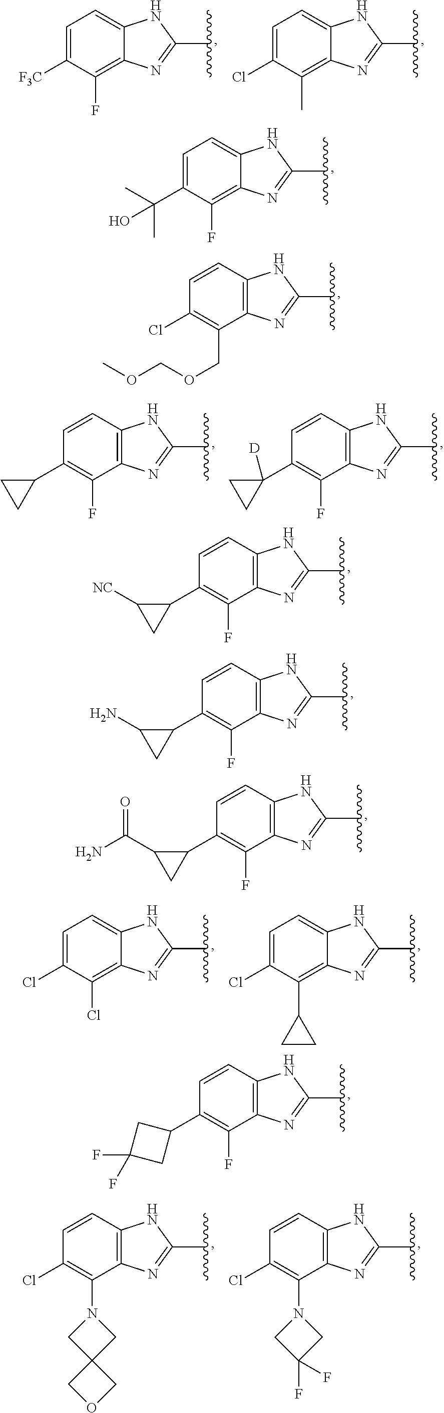

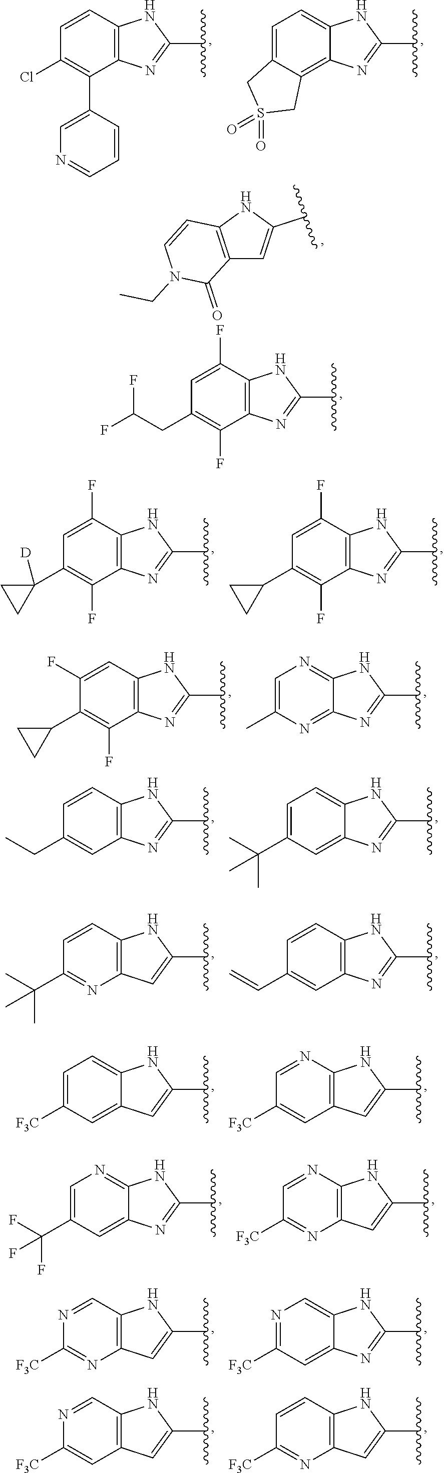

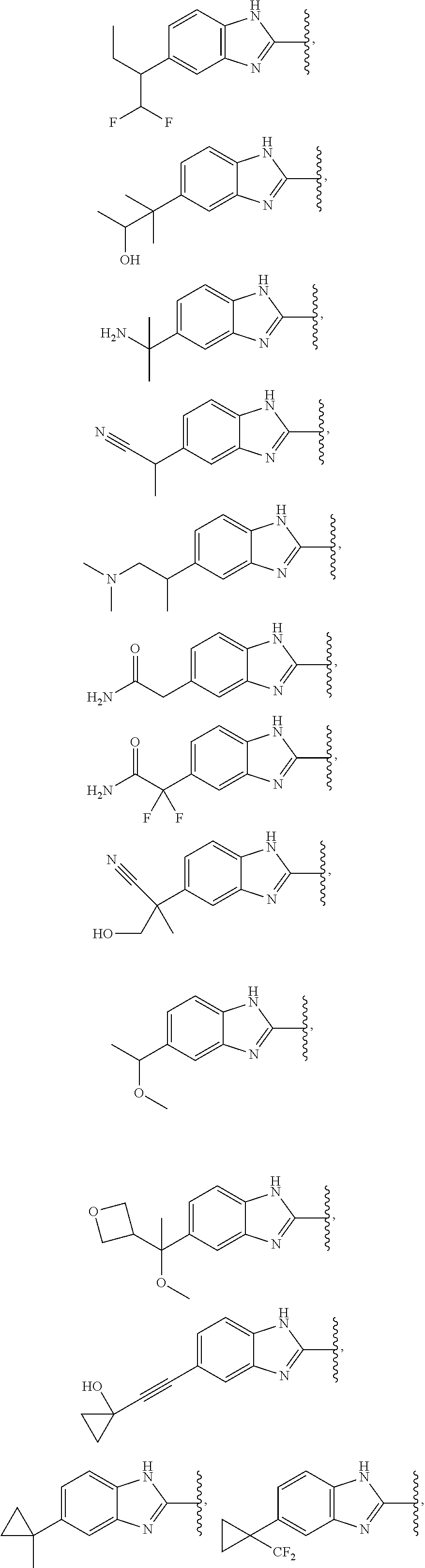

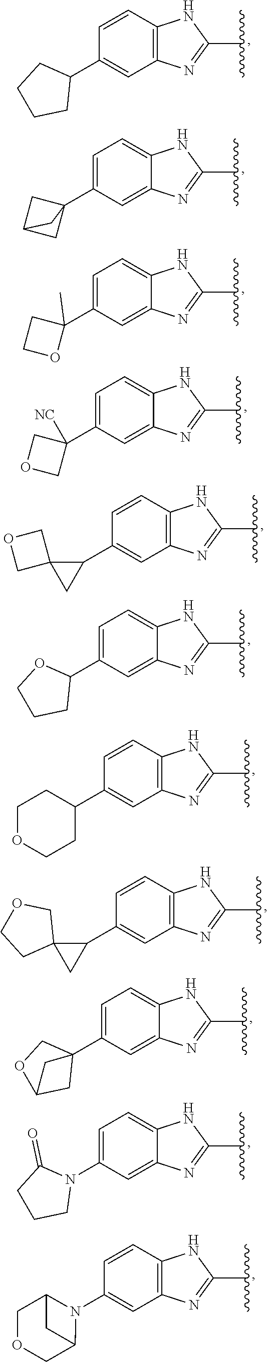

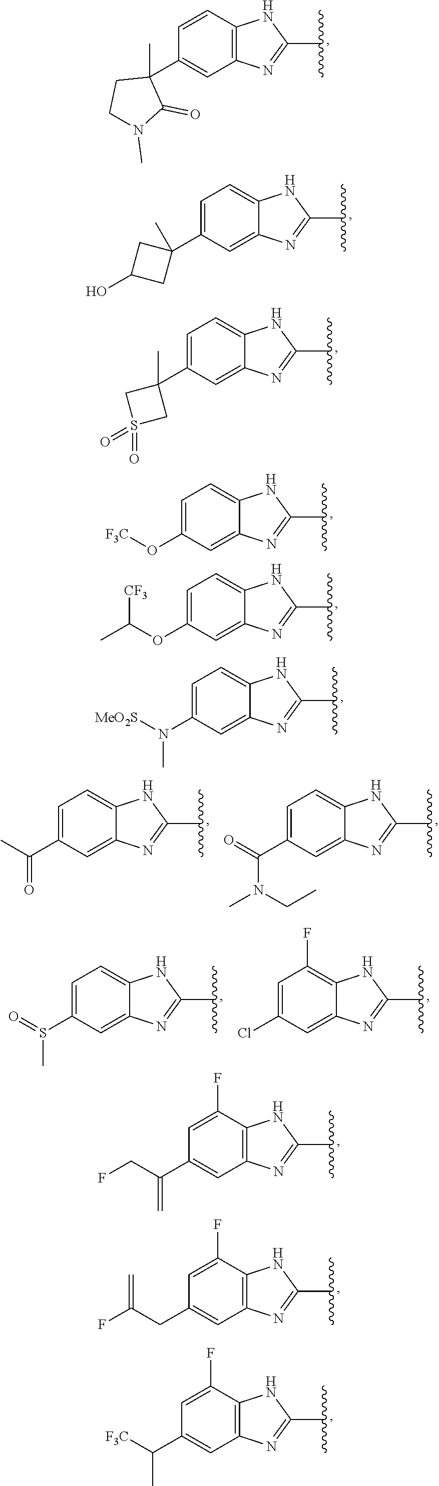

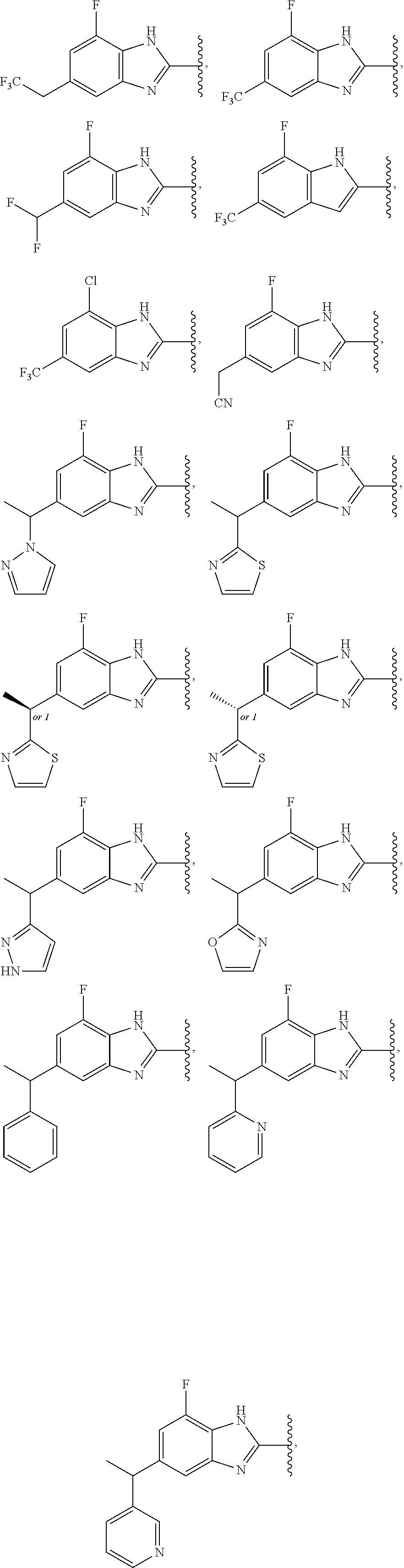

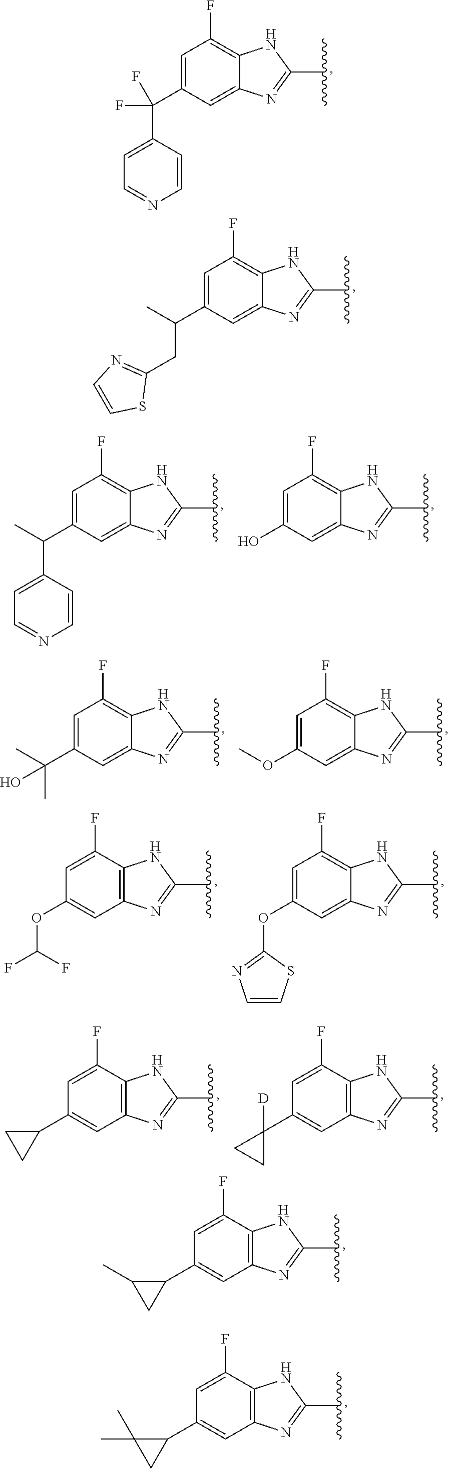

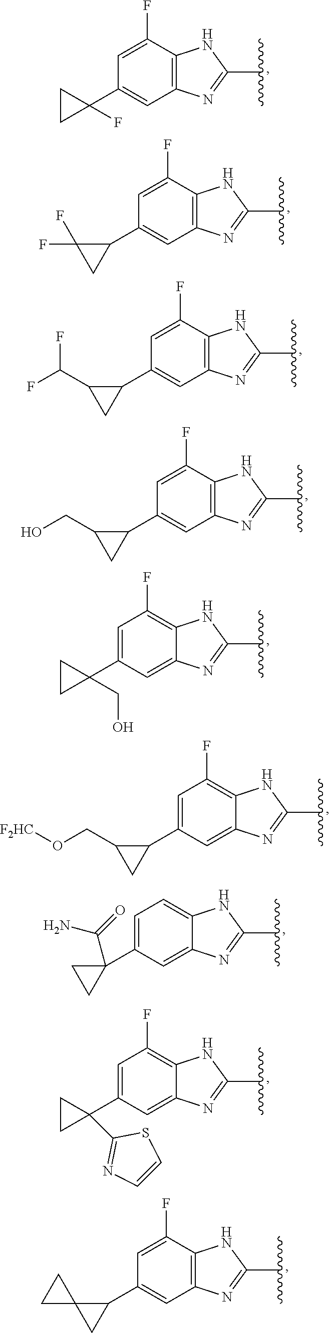

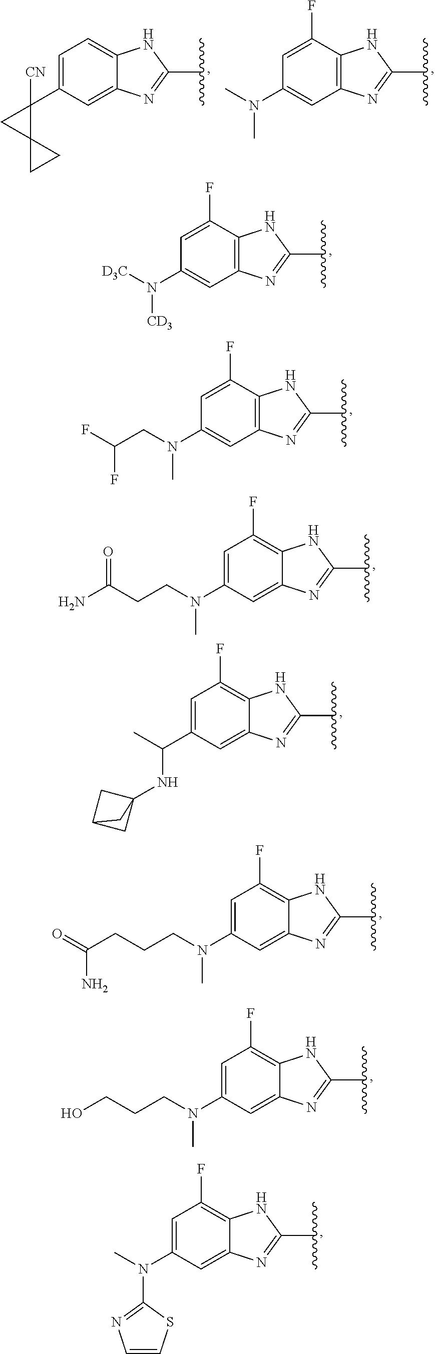

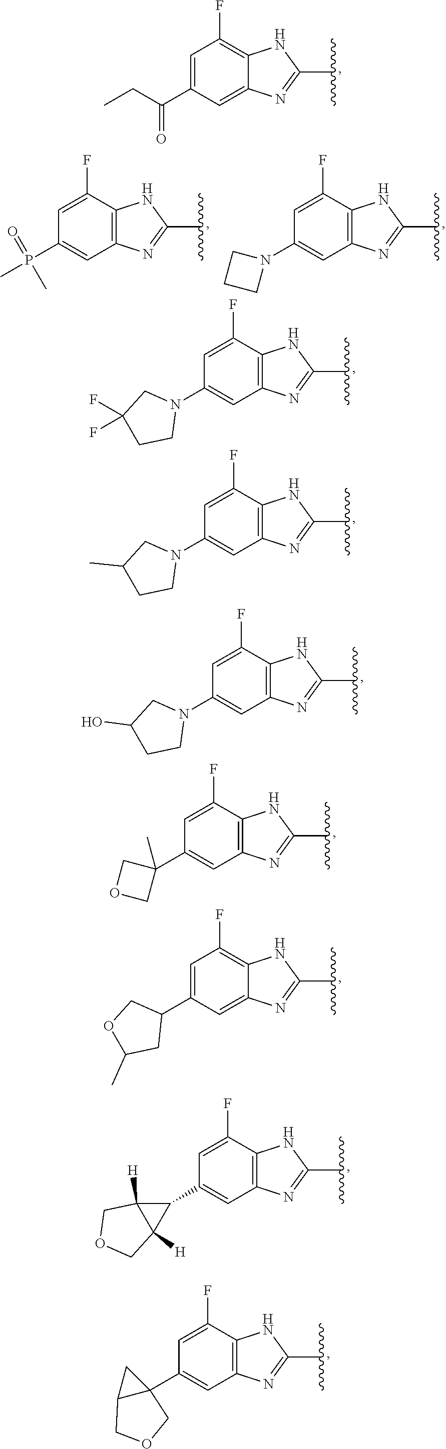

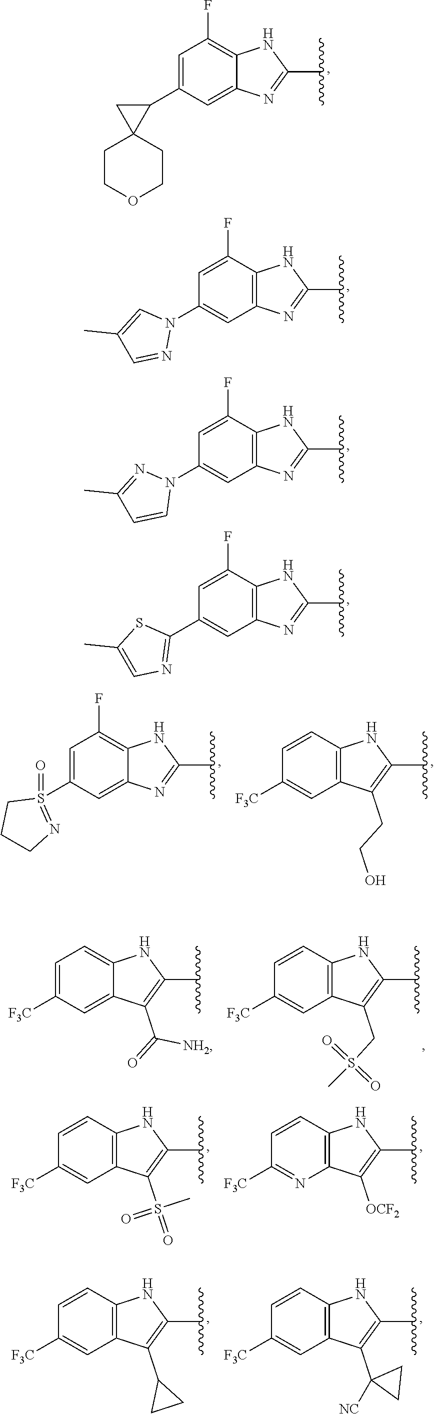

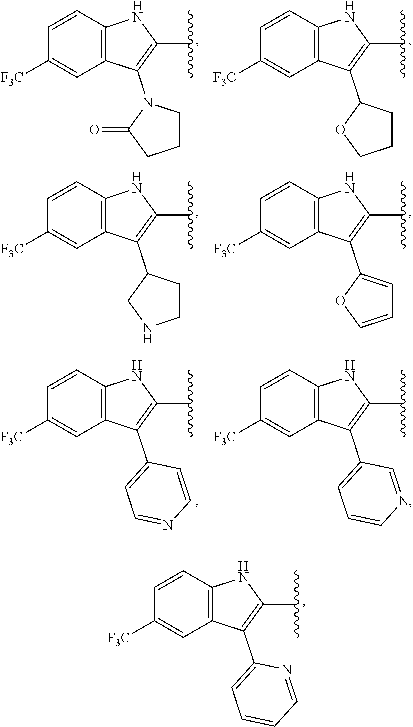

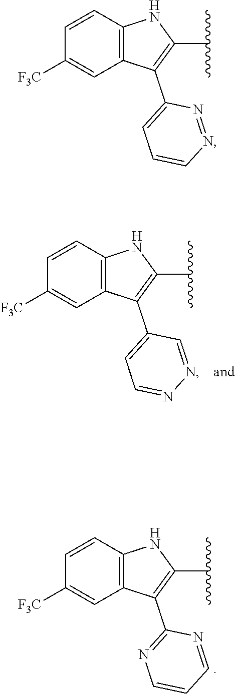 [SMILES: *c1[nH]c2ccc(C(F)(F)F)cc2c1-c1cccnn1 ; *c1[nH]c2ccc(C(F)(F)F)cc2c1-c1ccnnc1 ; *c1[nH]c2ccc(C(F)(F)F)cc2c1-c1ncccn1]

20. The compound, tautomer, solvate, stereoisomer, or pharmaceutically acceptable salt of claim 1, wherein Ring B is selected from: 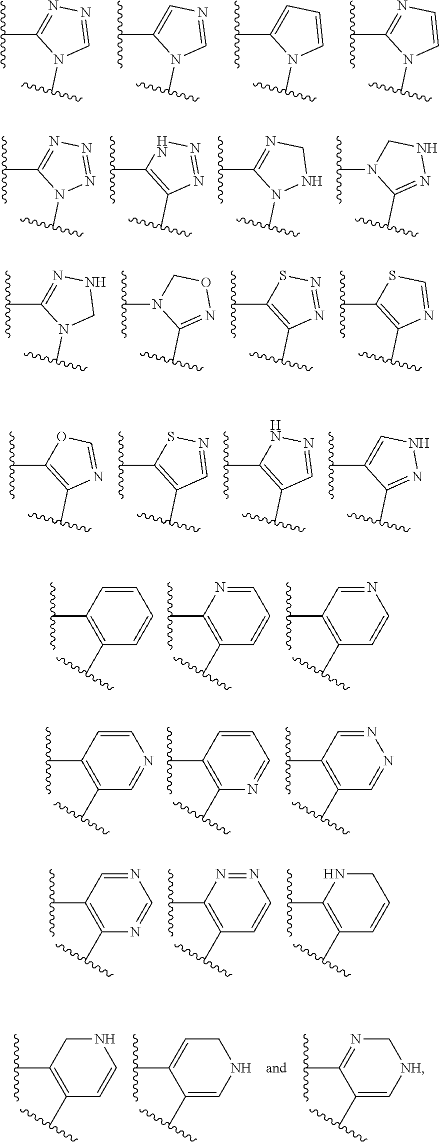
wherein Ring B is substituted with m groups of R⁶.

21. The compound, tautomer, solvate, stereoisomer, or pharmaceutically acceptable salt of claim 1, wherein Ring B is selected from: 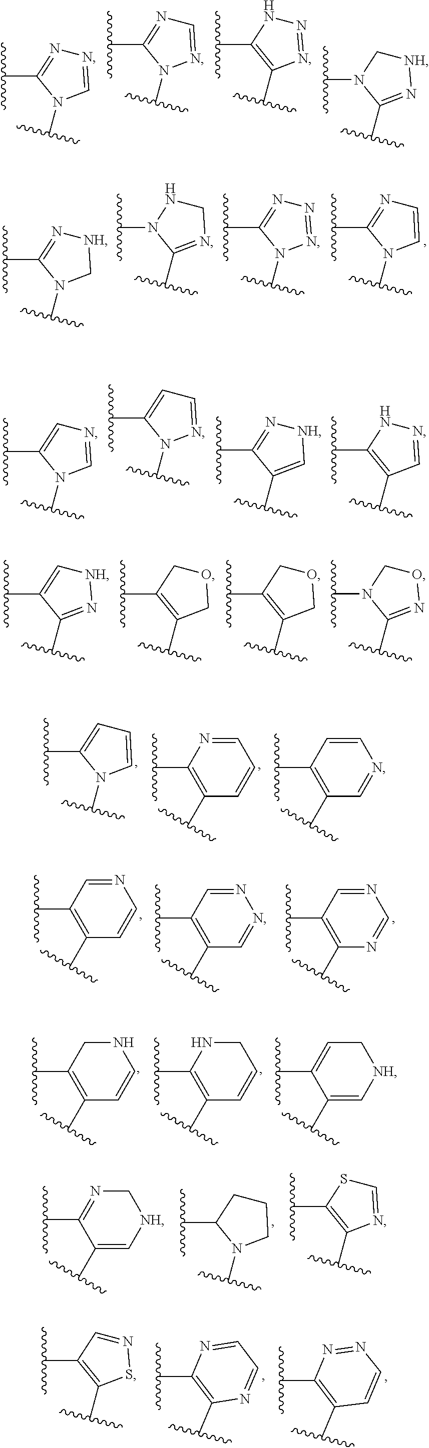 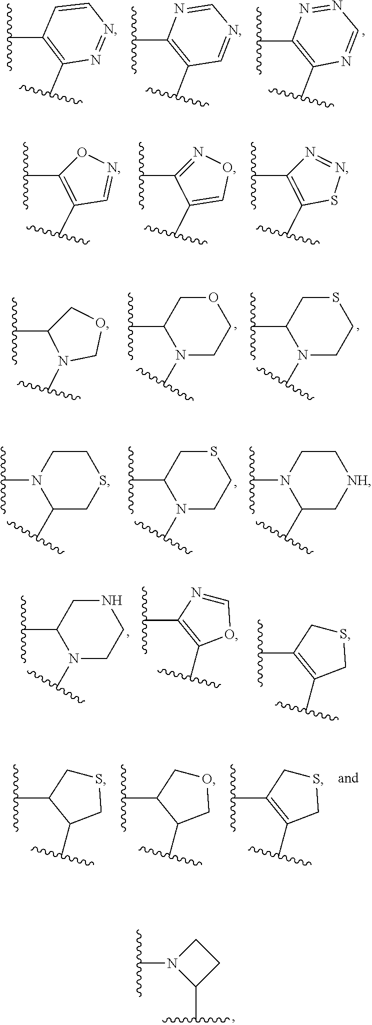
wherein Ring B is substituted with m groups of R⁶.

22. The compound, tautomer, solvate, stereoisomer, or pharmaceutically acceptable salt of claim 1, wherein 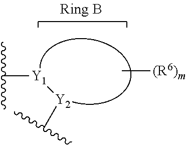
is selected from:

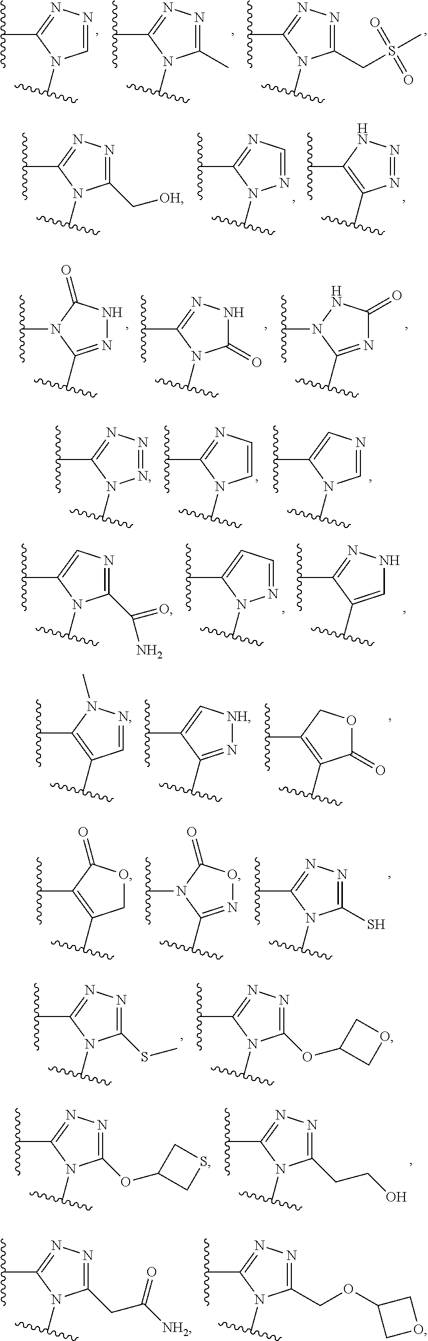

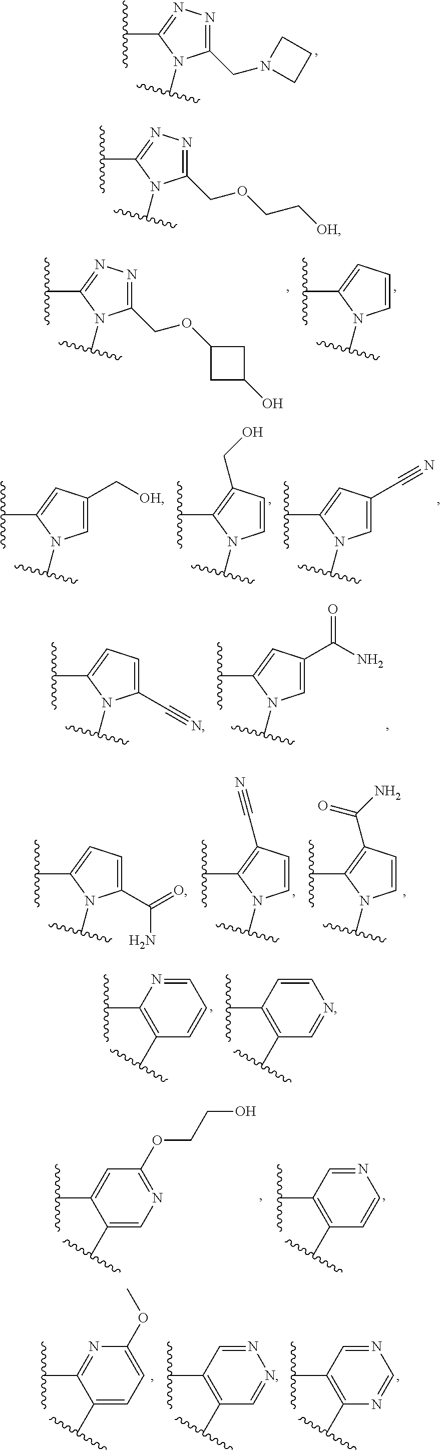

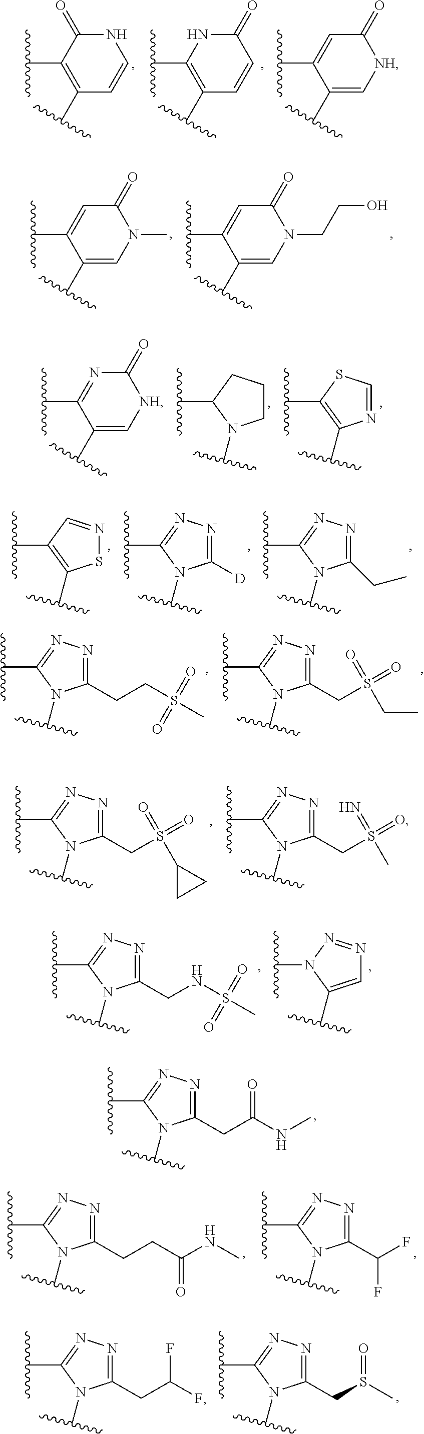

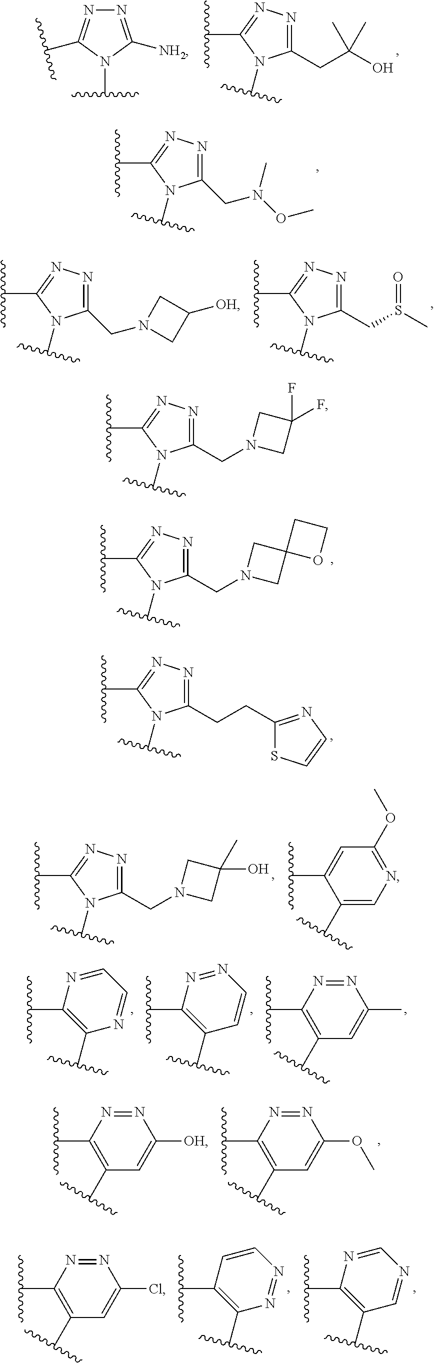

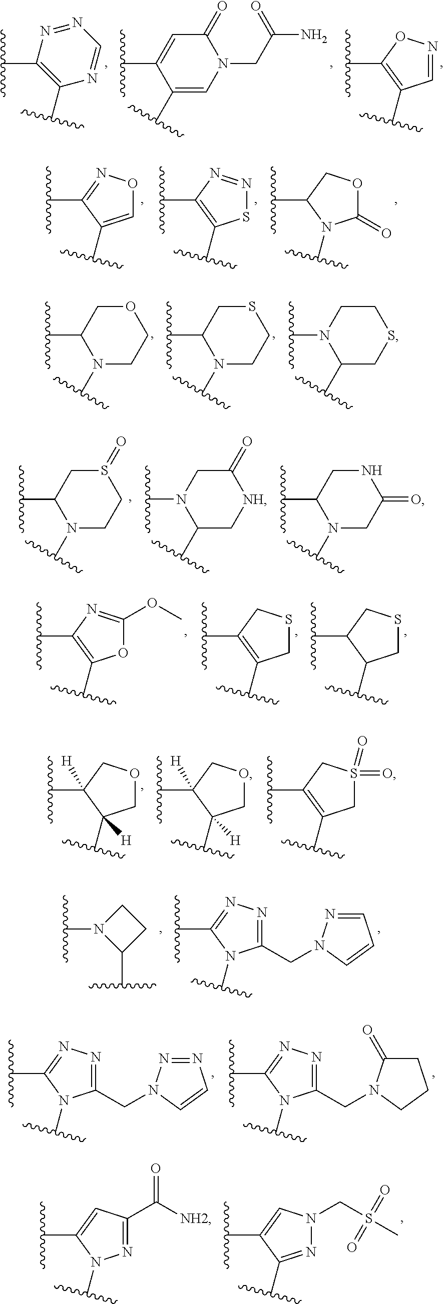

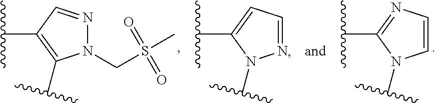

23. The compound, tautomer, solvate, stereoisomer, or pharmaceutically acceptable salt of claim 1, wherein Ring C is selected from: 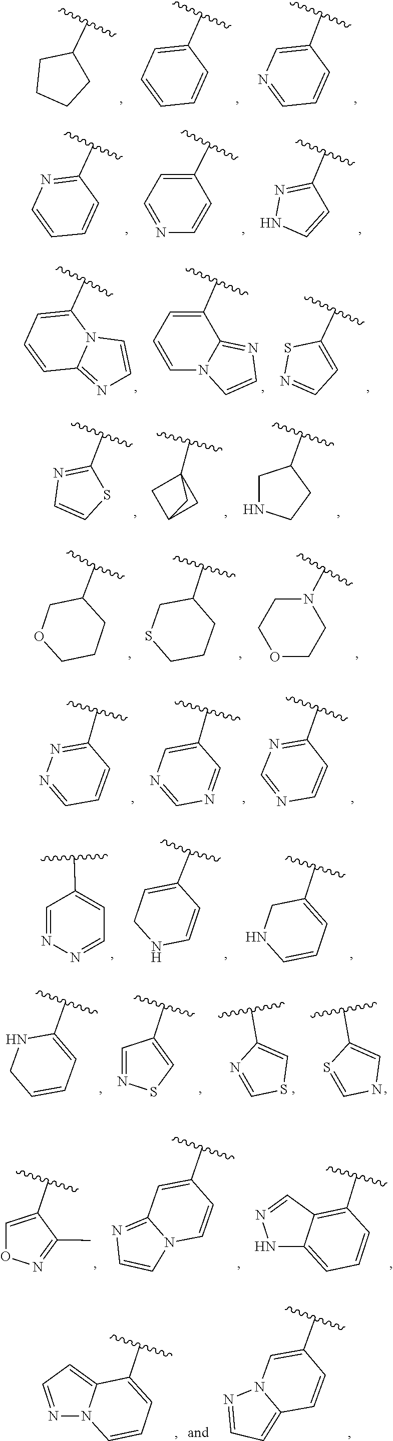
wherein Ring C is substituted with n groups of R⁷.

24. The compound, tautomer, solvate, stereoisomer, or pharmaceutically acceptable salt of claim 1, wherein 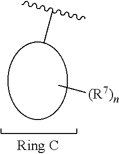
is selected from 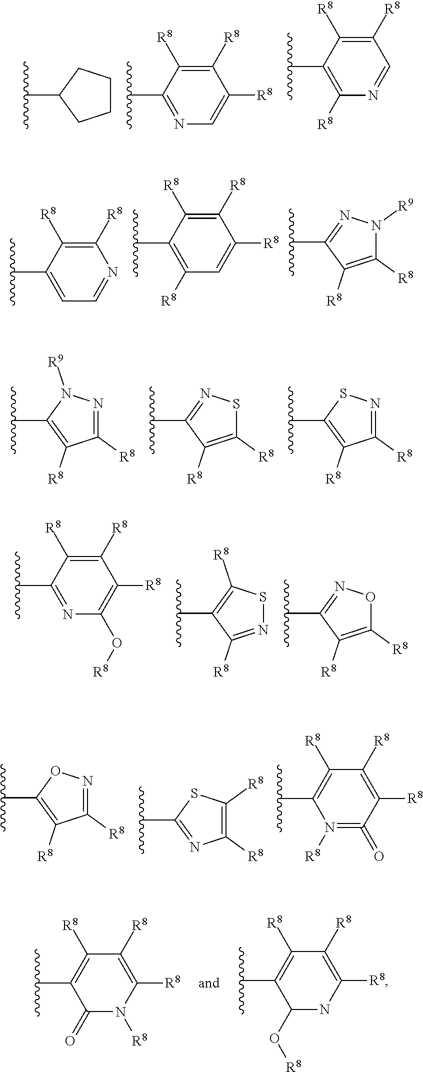
wherein:
R⁸, for each occurrence, is independently selected from H, F, Cl, Me, CHF₂, CF₃, CN, SO₂Me, SMe, CH₂CF₃, CH₂SO₂Me, 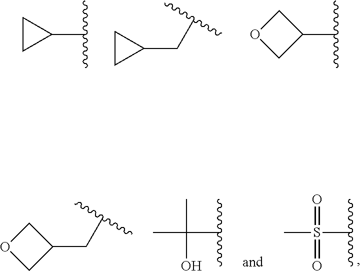
and
R⁹, for each occurrence, is independently selected from Me, CF₃, CHF₂, CH₂CF₃, Acetyl, SO₂Me,

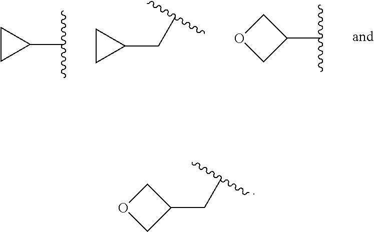

25. The compound, tautomer, solvate, stereoisomer, or pharmaceutically acceptable salt of claim 1, wherein 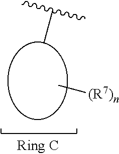
is selected from:

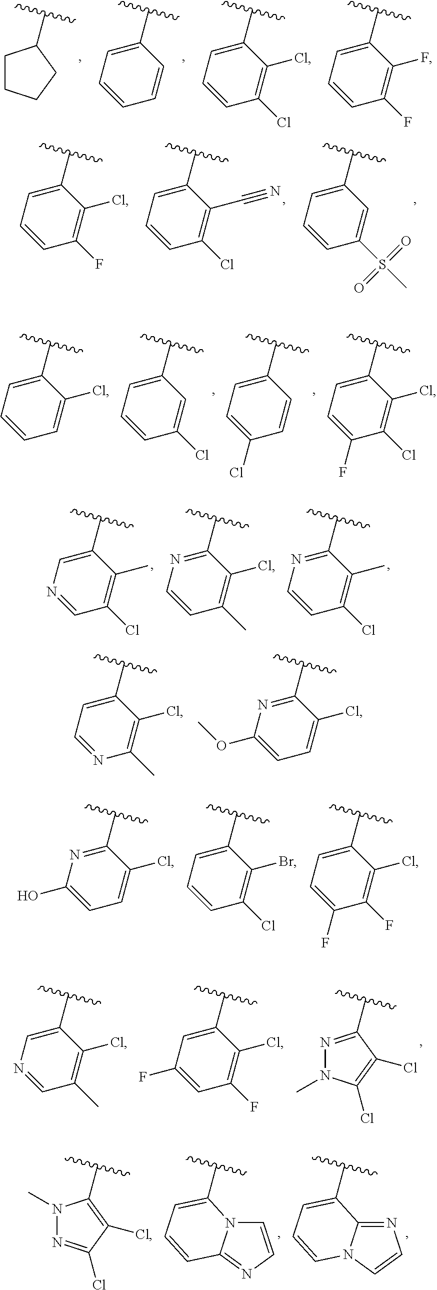

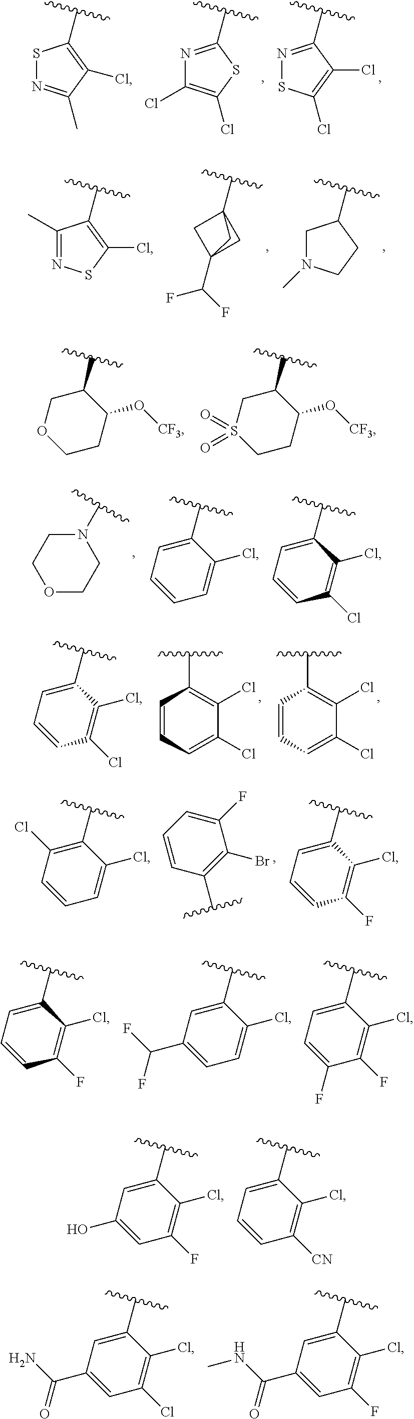

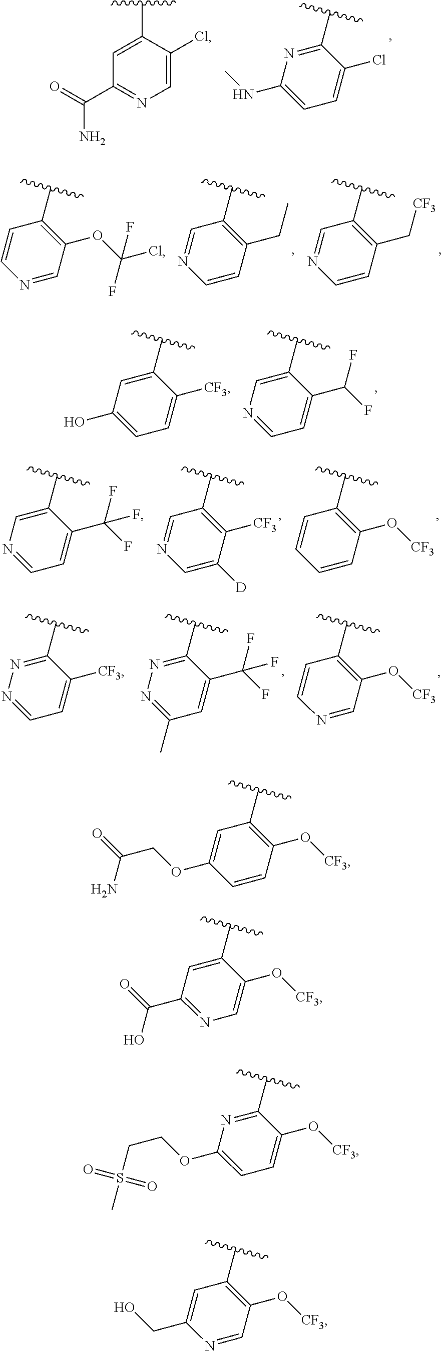

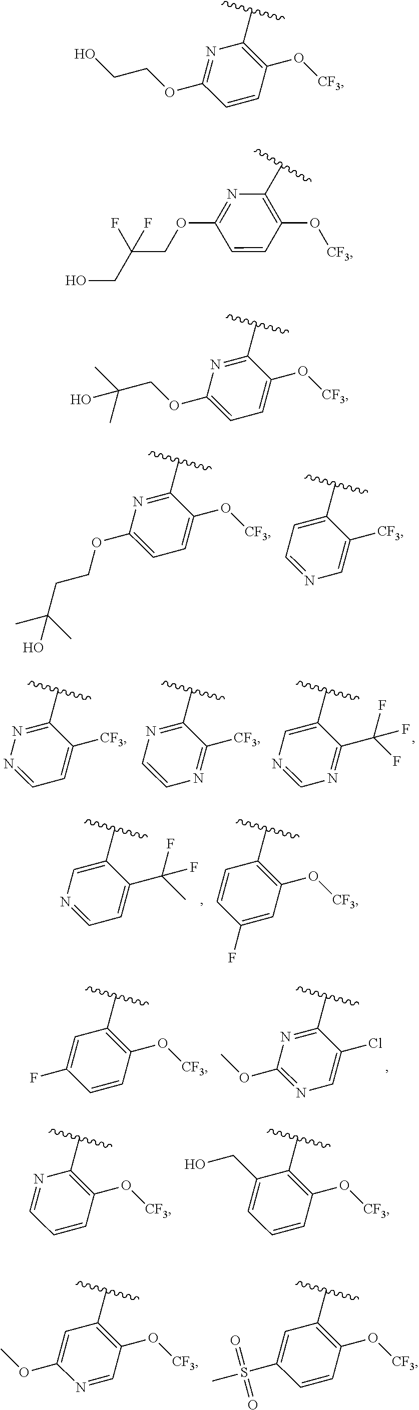

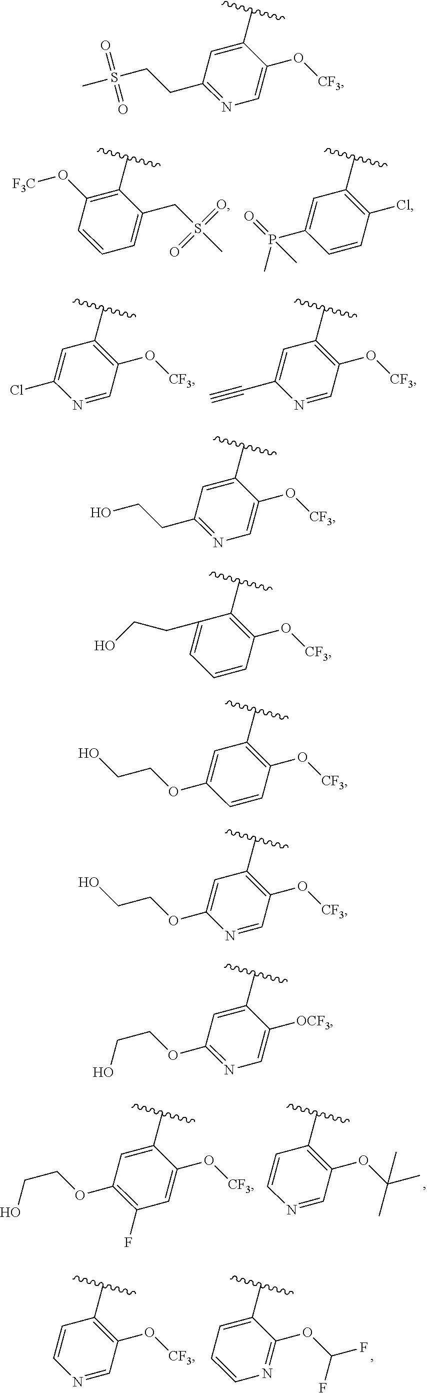

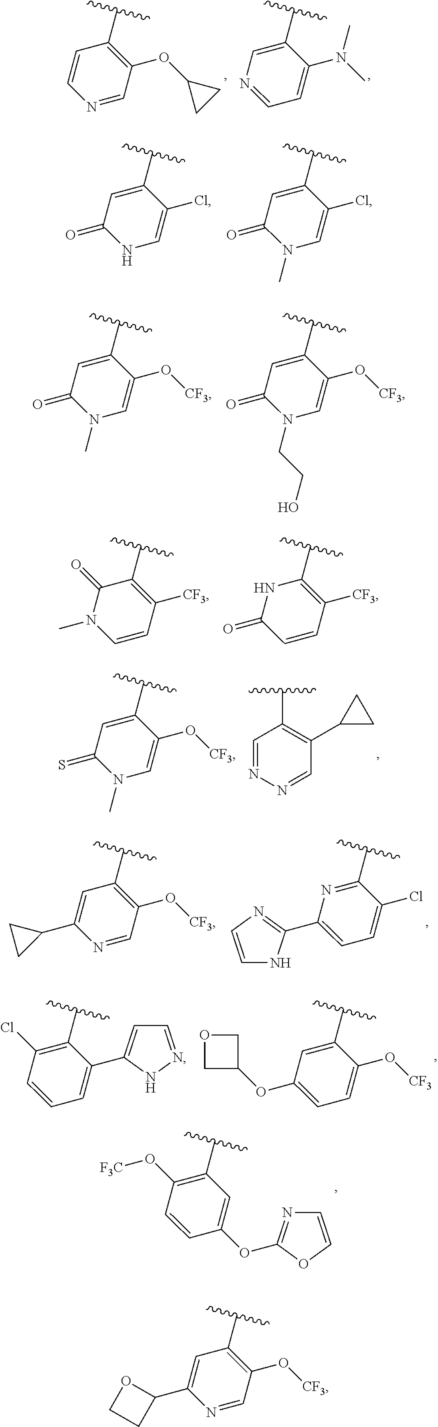

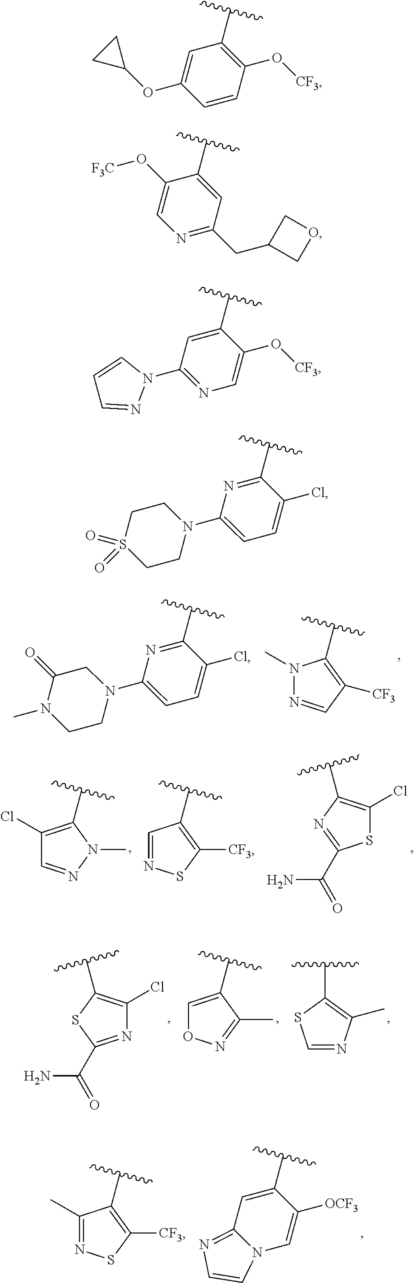

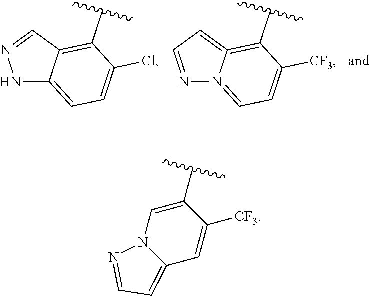

26. The compound, tautomer, solvate, stereoisomer, or pharmaceutically acceptable salt of claim 1, wherein R¹ is selected from: H, Me, Cl, F, Br, OMe, CF₃, OCF₃, CHF₂, SO₂Me, CN, OH, CH₂OH, COOH, CONH₂, 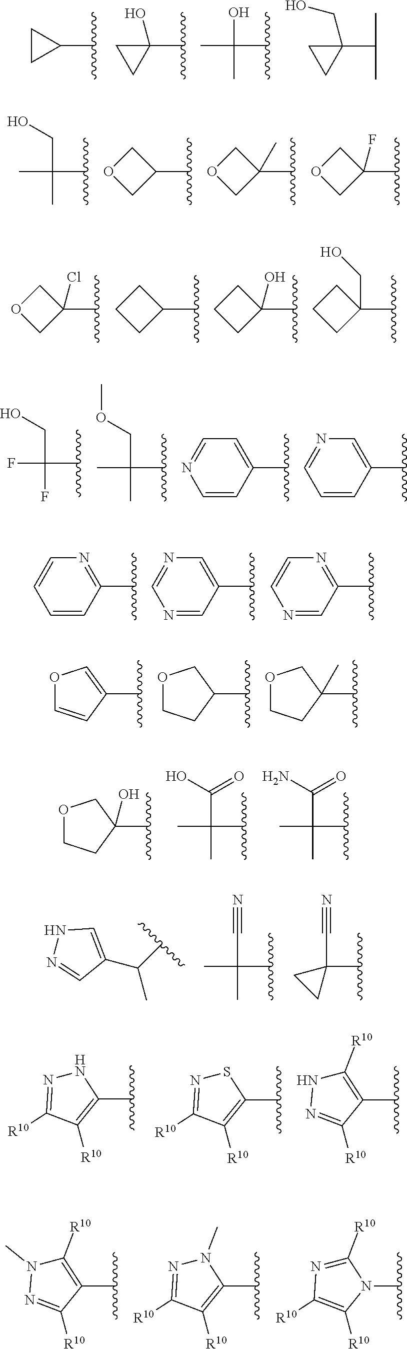 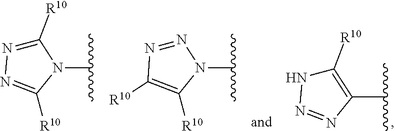
wherein R¹⁰, for each occurrence, is independently selected from H, Me, Cl, F, CF₃, and CN.

27. The compound, tautomer, solvate, stereoisomer, or pharmaceutically acceptable salt of claim 1, wherein R¹ is selected from: H, —OH₃, —OF₃, —OHF₂, —OCF₃, —C(CH₃)₂OH, Br, Cl, —S(═O)₂CH₃, —SF₅, 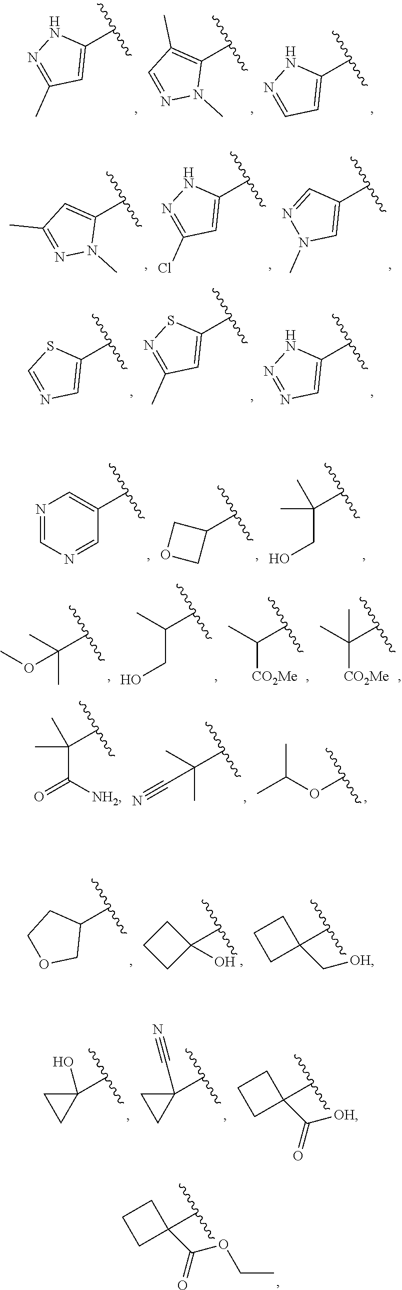
CH₂CH₃, C(CH₃)₃, —CH═CH₂, CH₂CHF₂, CH₂CF₃, CH₂OH, CH₂CN,

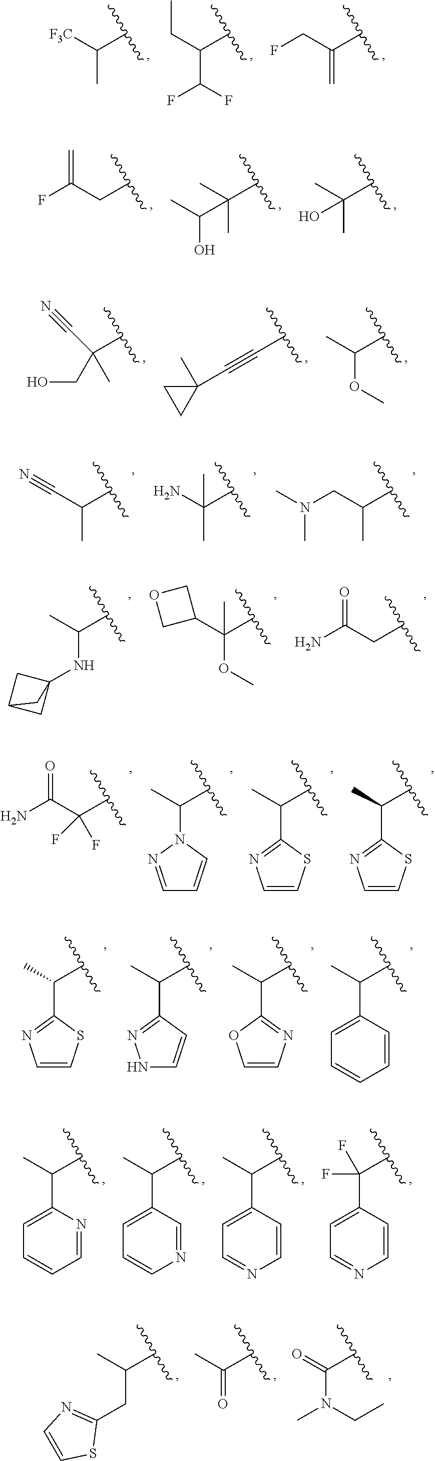

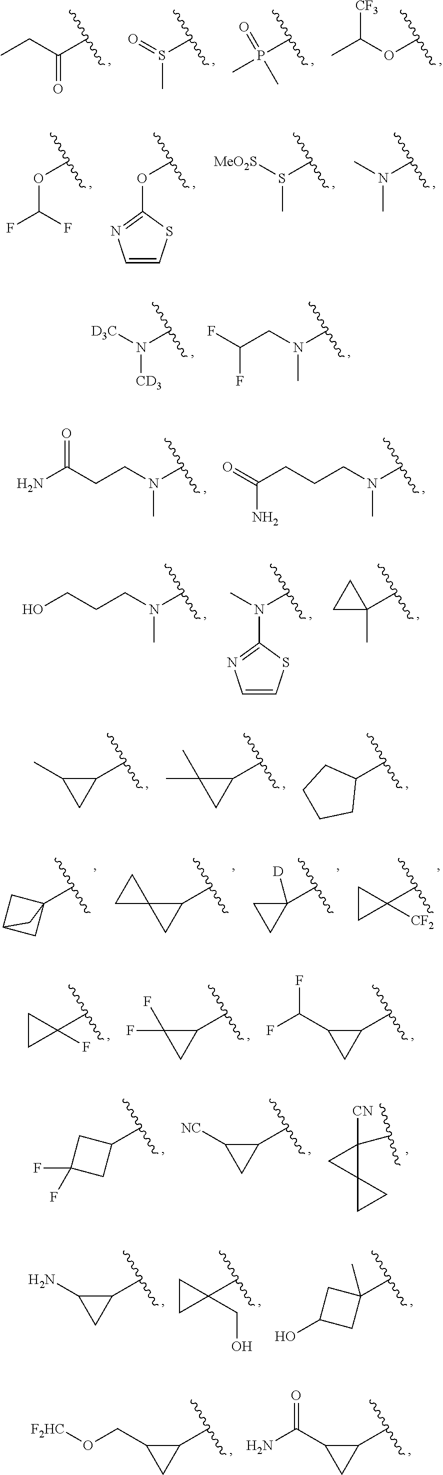

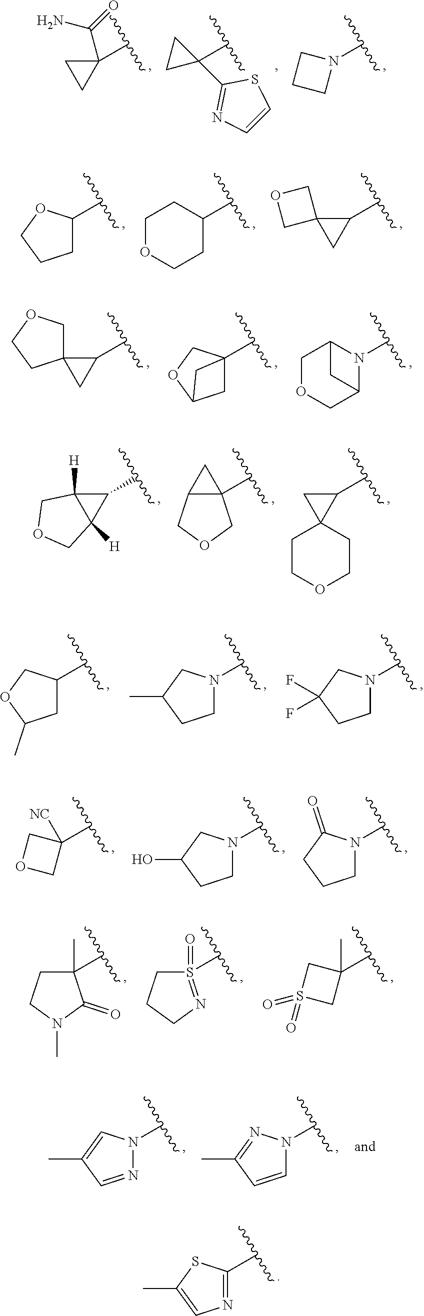

28. The compound, tautomer, solvate, stereoisomer, or pharmaceutically acceptable salt of claim 1, wherein R¹ is selected from: halogen, —C(═O)R^{f}, —OR^{f}, —NR^{f}R^{g}, —SF₅,
C₁-C₆ alkyl (optionally substituted with 1 to 3 groups selected from F, —CN, —OR^{f}, phenyl, —NR^{f}R^{g}, and 5- to 6-membered heteroaryl),
C₁-C₆ alkenyl (optionally substituted with 1 to 3 groups selected from F, —CN, —OR^{f}, phenyl, —NR^{f}R^{g}, and 5- to 6-membered heteroaryl),
3- to 6-membered cycloalkyl (optionally substituted with 1-2 groups selected from D, halogen, —CN, R^{f}, —OR^{f}, CH₂OR^{f}, —C(═O)NR^{f}R^{g}, and 5- to 6-membered heteroaryl), 4- to 8-membered heterocyclyl (optionally substituted with 1-2 groups selected from R^{f}, —OR^{f}, halogen, and —CN), and
5- to 6-membered heteroaryl (optionally substituted with 1-2 groups selected from R^{f}, —OR^{f}, halogen, and —CN),
wherein:
R^{f} and R^{g}, for each occurrence, are each independently selected from H, 5- to 6-membered heteroaryl, 3- to 6-membered cycloalkyl, and C₁-C₃ alkyl (optionally substituted with 1 to 3 groups selected from D, halogen, —OH, —OCH₃, —C(═O)NH₂, and —CN).

29. The compound, tautomer, solvate, stereoisomer, or pharmaceutically acceptable salt of claim 1, wherein R¹ is selected from: CF₃, F, Cl, C₁-C₃ alkyl and C₃-C₅ cycloalkyl.

30. The compound, tautomer, solvate, stereoisomer, or pharmaceutically acceptable salt of claim 1, wherein R² is selected from: H, Me, Cl, F, Br, —OMe, CF₃, —CN, —CONH₂, —SO₂Me, —S(═O)CH₃, —SCH₃, 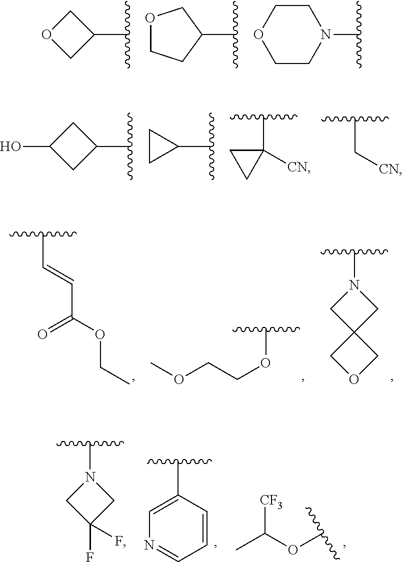
and —OH.

31. The compound, tautomer, solvate, stereoisomer, or pharmaceutically acceptable salt of claim 1, wherein R² is selected from: —CH₃, —S(═O)CH₃, —SCH₃, —CN, and —S(═O)₂CH₃.

32. The compound, tautomer, solvate, stereoisomer, or pharmaceutically acceptable salt of claim 1, wherein R³ and R⁴ are each independently selected from H, Me, Cl, F, Br, and OMe.

33. The compound, tautomer, solvate, stereoisomer, or pharmaceutically acceptable salt of claim 1, wherein R³ and R⁴ are each independently selected from H, Me, Cl, F, Br, OMe, CF₃, and

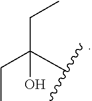 [SMILES: *C(O)(CC)CC]

34. The compound, tautomer, solvate, stereoisomer, or pharmaceutically acceptable salt of claim 1, wherein R⁴ is selected from F and Cl.

35. The compound, tautomer, solvate, stereoisomer, or pharmaceutically acceptable salt of claim 1, wherein R⁵ is selected from: —CN and —CH₂OH.

36. The compound, tautomer, solvate, stereoisomer, or pharmaceutically acceptable salt of claim 1, wherein R⁵ is selected from: absent, H, —CN, —CH₂OH, 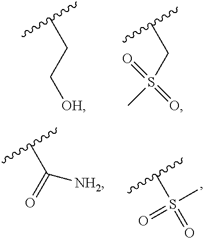
—OCHF₂,

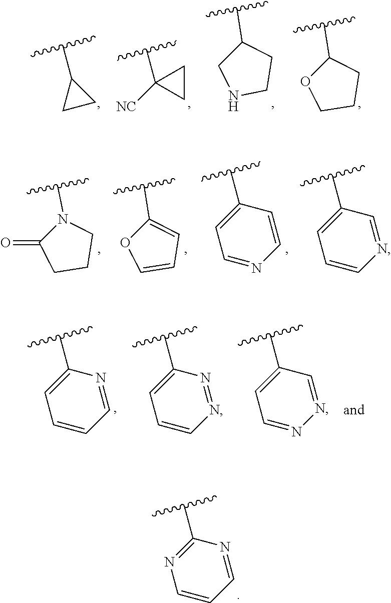

37. The compound, tautomer, solvate, stereoisomer, or pharmaceutically acceptable salt of claim 1, wherein R⁵ is selected from: absent, H, CN, halogen, —S(═O)₂CH₃, —CH₂S(═O)₂CH₃, 3- to 4-membered cycloalkyl, 5- to 6-membered heterocyclyl, 5- to 6-membered heteroaryl, CH₂OH, and CH₂CH₂OH.

38. The compound, tautomer, solvate, stereoisomer, or pharmaceutically acceptable salt of claim 1, wherein R⁶ is selected from: 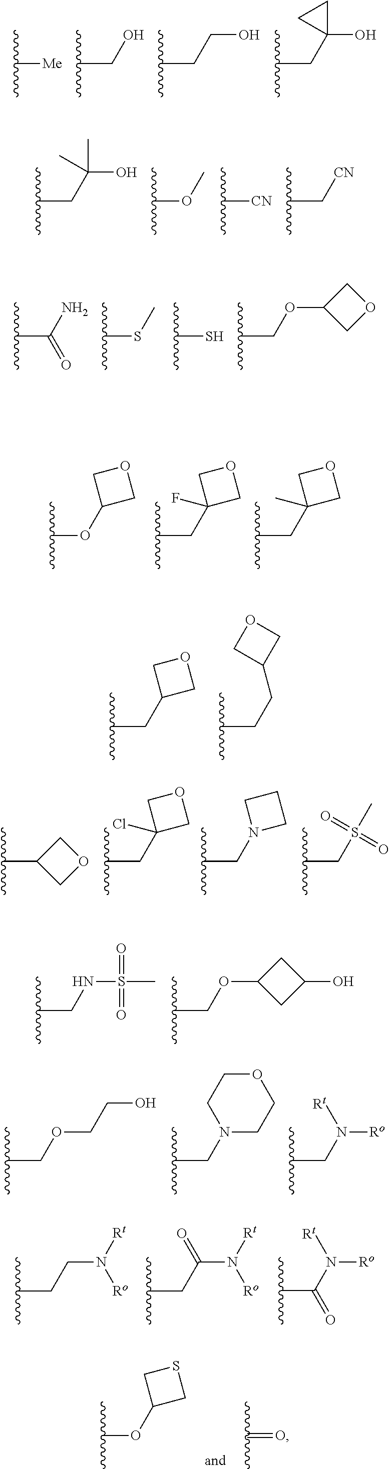
wherein R^{t} and R^{o} are each independently selected from H and C₁-C₆ alkyl, wherein the C₁-C₆ alkyl of R^{t} and R^{o} is optionally substituted with 1-3 groups selected from halogen.

39. The compound, tautomer, solvate, stereoisomer, or pharmaceutically acceptable salt of claim 1, wherein R⁶ is selected from: —CN, ═O, —CH₃, —CH₂S(═O)₂CH₃, —CH₂OH, —CH₂CH₂OH, —C(═O)NH₂, —O(CH₂)₂OH, —OCH₃, —SH, —SCH₃, 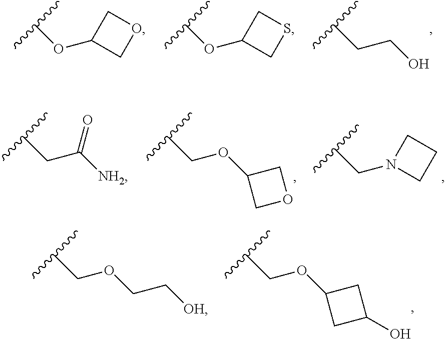
D, Cl, CH₂CH₃, CHF₂, CH₂CHF₂ 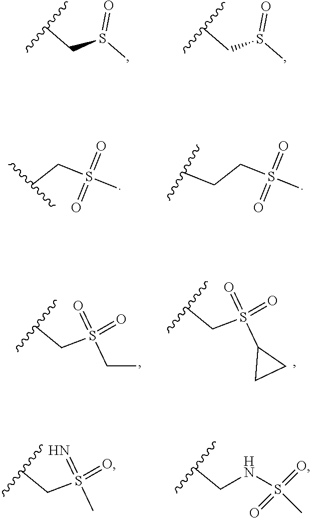
—CH₂CONH₂, 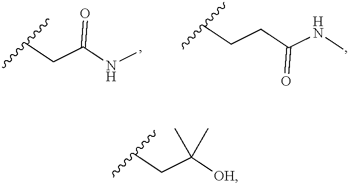
—NH₂, 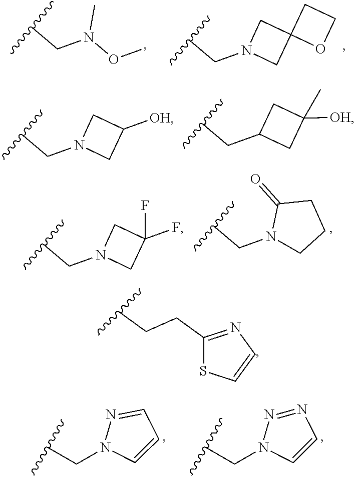
OH, —OCH₃, and ═O.

40. The compound, tautomer, solvate, stereoisomer, or pharmaceutically acceptable salt of claim 1, wherein R⁶ is selected from

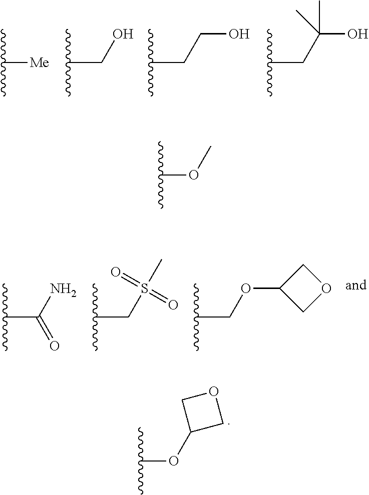

41. The compound, tautomer, solvate, stereoisomer, or pharmaceutically acceptable salt of claim 1, wherein R⁶ is selected from D, halogen, —S(═O)₂R^{h}, —NR^{h}Rⁱ, —C(═O)NR^{h}Rⁱ, and
C₁-C₄ alkyl (optionally substituted with 1 to 3 groups selected from halogen, —OR^{h}, —C(═O)NR^{h}Rⁱ, —NR^{h}Rⁱ, —S(═O)₂R^{h}, —NHS(═O)ₚR^{h}, 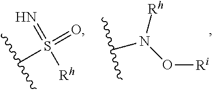
5- to 6-membered heteroaryl, 3- to 5-membered cycloalkyl (optionally substituted with 1 to 3 groups selected from halogen and R^{h}), and 3- to 8-membered heterocyclyl optionally substituted with 1 to 3 groups selected from halogen and R^{h}),
wherein:
R^{h} and Rⁱ, for each occurrence, are each independently selected from H, C₁-C₃ alkyl (optionally substituted with 1 to 3 groups selected from halogen, —OH, —O(C₁-C₃ alkyl), and —S(═O)₂CH₃), and 3- to 5-membered cycloalkyl.

42. The compound, tautomer, solvate, stereoisomer, or pharmaceutically acceptable salt of claim 1, wherein R⁷ is selected from F, Cl, Me, CHF₂, CF₃, CN, —SO₂Me, —SMe, CH₂CF₃, CH₂SO₂Me, acetyl, 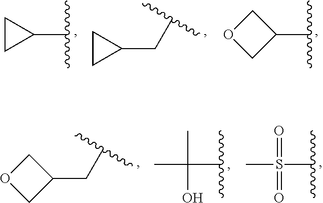
D, Br, CN, CH₂CH₃, 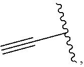 [SMILES: *C#C]
CH₂CF₃, CF₂CH₃, CH₂OH, CH₂CH₂OH, 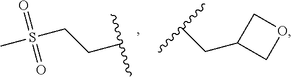
—OH, —OC(CH₃)₃, —OCF₃, —OCHF₂, —OCH₂CH₂OH, 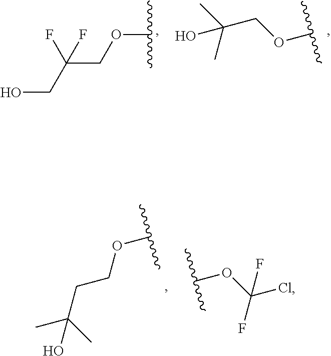
—OCH₂CONH₂, 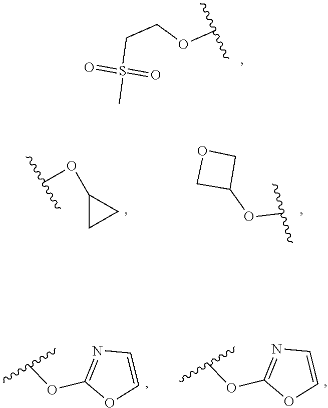
—COOH, —CONH₂, —CONHCH₃, ═O, ═S, —SO₂CH₃, 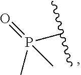 [SMILES: *P(C)(C)=O]
—NHCH₃, —N(CH₃)₂, 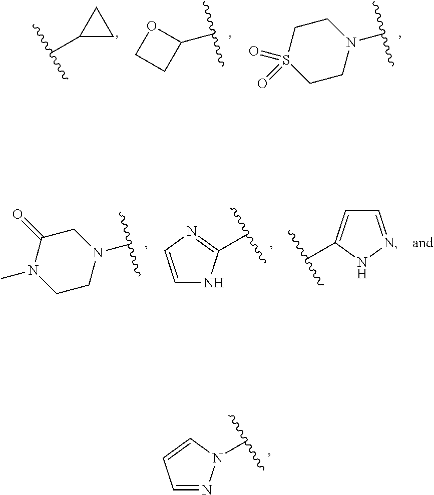
and N and n is 0, 1, 2, or 3.

43. The compound, tautomer, solvate, stereoisomer, or pharmaceutically acceptable salt of claim 1, wherein R⁷ is selected from: Cl, F, —CF₃, —OCF₃, —CN, —S(═O)₂CH₃, —CH₃, —OCH₃, and —OH.

44. The compound, tautomer, solvate, stereoisomer, or pharmaceutically acceptable salt of claim 1, wherein R⁷ is selected from: D, halogen, CN, ═O, ═S, —OR^{j}, —NR^{j}R^{k}, —C(═O)NR^{j}R^{k}, —C(═O)OR^{j}, —S(═O)₂CH₃, 3- to 5-membered cycloalkyl, 5- to 6-membered heteroaryl,
C₁-C₆ alkyl (optionally substituted with 1 to 3 groups selected from halogen, —OH, —S(═O)₂CH₃, and 4- to 6-membered heterocyclyl), and
4- to 6-membered heterocyclyl (optionally substituted with 1 to 2 groups selected from ═O and R^{k}),
wherein:
R^{j} and R^{k}, for each occurrence, are independently selected from H, C₁-C₆ alkyl (optionally substituted with 1 to 3 groups selected from halogen, OH, —C(═O)NH₂, and —S(═O)₂CH₃), 4- to 6-membered heterocyclyl, and 3- to 5-membered cycloalkyl.

45. The compound, tautomer, solvate, stereoisomer, or pharmaceutically acceptable salt of claim 1, wherein:
R¹ is selected from CH₃, CF₃, OCF₃, S(═O)₂CH₃, Br, Cl, 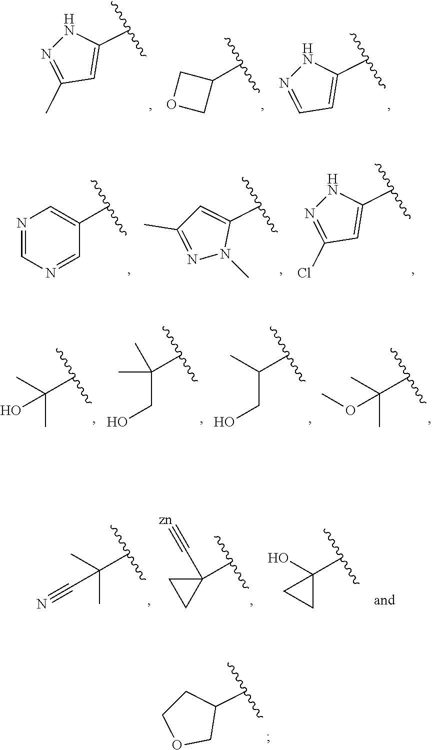
R² is selected from H, CN, and S(═O)₂CH₃,
R³, R⁴, and R⁵ are H,
R⁶ is selected from ═O, CH₃, Cl, —C(═O)NH₂, —CH₂CH₂OH, —CH₂OH, and —CH₂S(═O)₂CH₃,
m is 0, 1, or 2,
R⁷ is selected from CH₃, C, F, CN, OCH₃, and OH, and n is 0, 1, 2, or 3.

46. The compound, tautomer, solvate, stereoisomer, or pharmaceutically acceptable salt of claim 1, wherein:
R¹ is selected from C₁-C₆ alkyl (optionally substituted with 1 to 3 groups selected from halogen, —OH, CN, —OCH₃, —NR^{d}R^{e}, phenyl, 5-membered heteroaryl containing 1 to 3 heteroatoms selected from N, O, and S, and 6-membered heteroaryl containing 1 to 3 nitrogen atoms), C₂-C₄ alkenyl (optionally substituted with 1 to 3 groups selected from halogen, —OH, —CN, and —OCH₃), —OR^{d}, —NR^{d}R^{e}, —S(═O)ₚCH₃, —SF₅, halogen, —C(═O)CH₃, 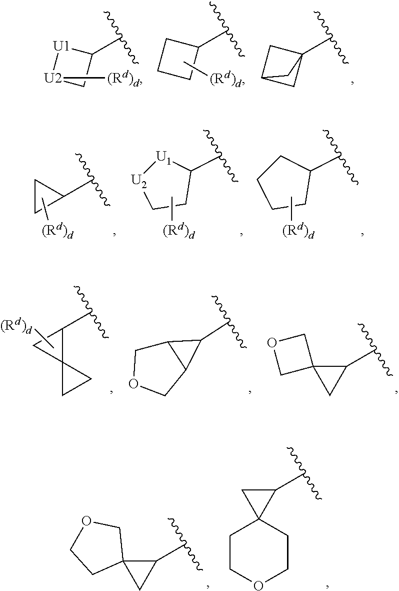
5-membered heteroaryl containing 1 to 2 heteroatoms selected from N and S (optionally substituted with 1 to 2 groups selected from C₁-C₃ alkyl), and 6-membered heteroaryl containing 1 to 2 nitrogen atoms (optionally substituted with 1 to 2 groups selected from C₁-C₃ alkyl);
R² is selected from H, CN, CH₃, F, and S(═O)₂CH₃; R³ is H; R⁴ is selected from F, Cl, and H;
R⁵ is selected from absent, H, F, —CN, —C(═O)NH₂, 3- to 4-membered cycloalkyl (optionally substituted with 1 to groups selected from CN and halogen), C₁-C₄ alkyl (optionally substituted with 1 to 3 groups selected from halogen, —S(═O)₂CH₃, and —OH), 5- to 6-membered heterocyclyl, —S(═O)₂CH₃, 5-membered heteroaryl containing 1 to 3 heteroatoms selected from N and 0, and 6-membered heteroaryl containing 1 to 3 nitrogen atoms;
R⁶ is selected from absent, D, C₁-C₄ alkyl (optionally substituted with 1-3 groups selected from halogen, —S(═O)CH₃, —S(═O)₂CH₃, —C(═O)NHCH₃, —OH, —C(═O)NH₂, —NR^{d}R^{e}, —NR^{d}OR^{e}, and —NHS(═O)₂CH₃), ═O, C, and —C(═O)NH₂;
R⁷ is selected from D, halogen, CF₃, —OCF₃, CN, —OR^{d}, —NR^{d}R^{e}, —C(═O)OH, ═O, ═S, —S(═O)₂CH₃, —C(═O)NR^{d}R^{e}, C₁-C₆ (optionally submitted with 1-3 groups selected from halogen, —OH, —S(═O)₂CH₃, —C(═O)₂NH₂, and 3- to 6-membered heterocyclyl), 5- to 6-membered heteroaryl, 3- to 5-membered cycloalkyl, and 4- to 6-membered heterocyclyl (optionally substituted with 1 to 3 groups selected from ═O and C₁-C₃ alkyl);
wherein:
R^{d} and R^{e}, for each occurrence, are each independently selected from H, C₁-C₄ alkyl (optionally substituted with 1 to 3 groups selected from D, halogen, —OH, CN, —C(═O)NH₂, —S(═O)₂CH₃, and —OCH₃), 5- to 6-membered heteroaryl, 4- to 6-membered heterocyclyl, and 3- to 5-membered cycloalkyl;
q is 0, 1, 2, or 3;
U₁ and U₂ are independently selected from O and C.

47. The compound according to claim 1, wherein the compound is selected from Compounds 1 to 468, a tautomer thereof, a solvate or stereoisomer of the compound or the tautomer, or a pharmaceutically acceptable salt of the foregoing.

48. A pharmaceutical composition comprising a compound according to claim 1, a tautomer thereof, a solvate or stereoisomer of the compound or the tautomer, or a pharmaceutically acceptable salt of the foregoing and at least one pharmaceutically acceptable carrier.

49. A method of treating a disease or condition, comprising administering to a subject in need thereof, a therapeutically effective amount of a compound according to claim 1, a tautomer thereof, a solvate or stereoisomer of the compound or the tautomer, or a pharmaceutically acceptable salt of the foregoing, or a pharmaceutical composition comprising a compound according to claim 1, a tautomer thereof, a solvate or stereoisomer of the compound or the tautomer, or a pharmaceutically acceptable salt of the foregoing and at least one pharmaceutically acceptable carrier, wherein the disease or condition is selected from ALS, Parkinson's disease, multiple sclerosis, traumatic brain injury, diabetic neuropathy, and CIPN.

50. A method of treating a disease or condition associated with axonal degeneration, comprising administering to a subject in need thereof, a therapeutically effective amount of a compound according to claim 1, a tautomer thereof, a solvate or stereoisomer of the compound or the tautomer, or a pharmaceutically acceptable salt of the foregoing, or a pharmaceutical composition comprising a compound according to claim 1, a tautomer thereof, a solvate or stereoisomer of the compound or the tautomer, or a pharmaceutically acceptable salt of the foregoing and at least one pharmaceutically acceptable carrier.

51. A method of modulating SARM1, comprising contacting a subject in need thereof with a compound according to claim 1, a tautomer thereof, a solvate or stereoisomer of the compound or the tautomer, or a pharmaceutically acceptable salt of the foregoing, or a pharmaceutical composition comprising a compound according to claim 1, a tautomer thereof, a solvate or stereoisomer of the compound or the tautomer, or a pharmaceutically acceptable salt of the foregoing and at least one pharmaceutically acceptable carrier.

52. A method of inhibiting or preventing axonal degeneration, comprising contacting a subject in need thereof with a compound according to claim 1, a tautomer thereof, a solvate or stereoisomer of the compound or the tautomer, or a pharmaceutically acceptable salt of the foregoing, or a pharmaceutical composition comprising a compound according to claim 1, a tautomer thereof, a solvate or stereoisomer of the compound or the tautomer, or a pharmaceutically acceptable salt of the foregoing and at least one pharmaceutically acceptable carrier.
